(19) United States
(12) Patent Application Publication
(10) Pub. No.: US 2026/0062420 A1
(43) Pub. Date: Mar. 5, 2026

(54) NOVEL DPP1 INHIBITORS AND USES THEREOF

(71) Applicant: Insmed Incorporated, Bridgewater, NJ (US)

(72) Inventors: Adam J. PLAUNT, Bridgewater, NJ (US); David C. CIPOLLA, Bridgewater, NJ (US); Adrien CERDAN, Strasbourg (FR); Paola CIAPETTI, Strasbourg (FR); Bastien GOEGAN, Strasbourg (FR); David J. AUGERI, Princeton, NJ (US)

(21) Appl. No.: 18/997,734

(22) PCT Filed: Jul. 27, 2023
(86) PCT No.: PCT/US2023/071162

§ 371 Date: Jan. 22, 2025

Related U.S. Application Data
(60) Provisional application No. 63/369,750, filed on Jul. 28, 2022.
(60) Provisional application No. 63/465,517, filed on May 10, 2023.
(60) Provisional application No. 63/468,978, filed on May 25, 2023.

(51) Int. Cl. C07D 491/107; A61K 31/397; A61K 31/403; A61K 31/423; A61K 31/437; A61K 31/439; A61K 31/4439; A61K 31/454; A61K 31/496; A61K 31/5377; A61K 31/5386; A61K 31/541; A61K 31/553; A61K 31/554; C07B 59/00; C07D 205/04; C07D 209/52; C07D 263/58; C07D 267/10; C07D 409/12; C07D 413/12; C07D 413/14; C07D 417/12; C07D 419/12; C07D 453/02; C07D 471/04; C07D 487/08; C07D 491/048; C07D 497/10

(52) U.S. Cl. CPC C07D 491/107; A61K 31/397; A61K 31/403; A61K 31/423; A61K 31/437; A61K 31/439; A61K 31/4439; A61K 31/454; A61K 31/496; A61K 31/5377; A61K 31/5386; A61K 31/541; A61K 31/553; A61K 31/554; C07B 59/002; C07D 205/04; C07D 209/52; C07D 263/58; C07D 267/10; C07D 409/12; C07D 413/12; C07D 413/14; C07D 417/12; C07D 419/12; C07D 453/02; C07D 471/04; C07D 487/08; C07D 491/048; C07D 497/10; C07B 2200/05

(57) ABSTRACT

Provided herein are novel DPP1 inhibitor compounds having the general structure of Formula (I), (II) or (III), or a pharmaceutically acceptable salt thereof. Also provided are certain methods of treatment, e.g., a method for treating an obstructive disease of the airway or a method of treating an inflammatory condition with a composition comprising an effective amount of one of the compounds provided herein.

### CROSS-REFERENCE TO RELATED APPLICATIONS

This application claims priority to U.S. Provisional Application No. 63/369,750, filed Jul. 28, 2022, U.S. Provisional Application No. 63/465,517, filed May 10, 2023, and U.S. Provisional Application No. 63/468,978, filed May 25, 2023, the disclosure of each of which is incorporated by reference in its entirety for all purposes.

### BACKGROUND OF THE INVENTION

Dipeptidyl peptidase 1 (DPP1; EC 3.4.14.1), also known as cathepsin C, is a lysosomal cysteine protease belonging to the papain family having a molecular weight of 200 kDa. DPP1 was first discovered by Gutman and Fruton in 1948 (J Biol Chem, 174, 851-858); however, the cDNA of the human enzyme was first described in 1995 (Paris et al. 1995, FEBS Lett, 369, 326-330). DPP1 is the only member of the papain family that is functional as a tetramer, consisting of four identical subunits. Each subunit is composed of an N-terminal fragment, a heavy chain and a light chain (Dolenc et al. 1995, J Biol Chem, 270, 21626-21631).

DPP1 is constitutively expressed in many tissues with highest levels in lung, kidney, liver and spleen. DPP1 catalyzes the removal of dipeptides from the N-terminal end of polypeptide substrates with broad specificity. Recent data suggest that besides being an important enzyme in lysosomal protein degradation, DPP1 also functions as a key enzyme in the activation of granule serine proteases in cytotoxic T-lymphocytes and natural killer cells (granzymes A and B), mast cells (chymase and tryptase) and neutrophils (cathepsin G, neutrophil elastase and proteinase-3).

Mast cells are found in many tissues but are present in greater numbers along the epithelial linings of the body, such as the skin, respiratory tract and gastrointestinal tract. In humans, two types of mast cells have been identified. The T-type, which expresses only tryptase, and the MC-type, which expresses both tryptase and chymase. In humans, the T-type mast cells are located primarily in alveolar tissue and intestinal mucosa while the TC-type cells predominate in skin and conjunctiva. Tryptase and chymase appear to be important mediators of allergic diseases, being involved in processes of inflammation, bronchoconstriction and mucus secretion.

Neutrophils play a critical role in host defense against invading pathogens. Neutrophils are produced in the bone marrow and are fully mature when released into the circulation to take up their role as the first line of cellular defense. Pro-inflammatory mediators and chemotactic attractants activate neutrophils and draw them to the site of infection, where they act to engulf bacteria by phagocytosis, assaulting them with an arsenal of anti-bacterial compounds that use both oxidative and non-oxidative methods of attack. The powerful serine protease, neutrophil elastase, is one of those anti-bacterial compounds that are clearly involved in destroying bacteria. Neutrophil elastase is released into the phagolysome surrounding the microorganism, which it proceeds to destroy. Neutrophil elastase is able to attack the outer membrane protein, OmpA, in gram-negative bacteria, helping to directly kill the pathogen by degrading its membrane, as well as enabling other anti-bacterial compounds to gain access to the pathogen. In addition, neutrophil elastase may help process other antibacterial compounds, converting them from inactive pro-peptides into their active states, such as for cathelicidin.

Yet neutrophil elastase can also cause problems for its host. It is one of the most destructive enzymes in the body, with the capability of degrading extracellular matrix proteins (including collagens, proteoglycan, fibronectin, platelet receptors, complement receptor, thrombomodulin, lung surfactant and cadherins) and key plasma proteins (including coagulation and complement factors, immunoglobulin, several proteases and protease inhibitors). Under physiological conditions, endogenous protease inhibitors, such as α1-antitrypsin, tightly regulate the activity of neutrophil elastase. However, at inflammatory sites, neutrophil elastase is able to evade regulation, and once unregulated it can induce the release of pro-inflammatory cytokines, such as interleukin-6 and interleukin-8, leading to acute lung injury. It can even impair host defense against infection by degrading phagocyte surface receptors and opsonins. Its negative role is illustrated by its involvement in the tissue destruction and inflammation that characterize numerous diseases, including hereditary emphysema, chronic obstructive pulmonary disease, cystic fibrosis, adult respiratory distress syndrome, ischemic-reperfusion injury and rheumatoid arthritis.

As such, there is a need in the art to provide novel DPP1 inhibitors in order to treat the aforementioned diseases, and others associated with DPP1 and neutrophil elastase.

### SUMMARY OF THE INVENTION

In one aspect of the invention, a DPP1 inhibitor compound of Formula (I), or a pharmaceutically acceptable salt thereof, is provided:

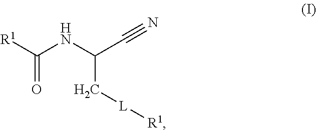

wherein,
R¹ is

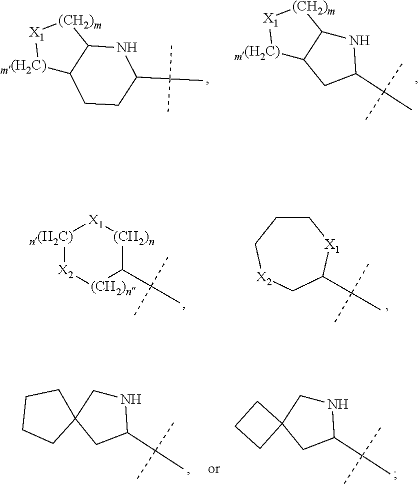

R² is

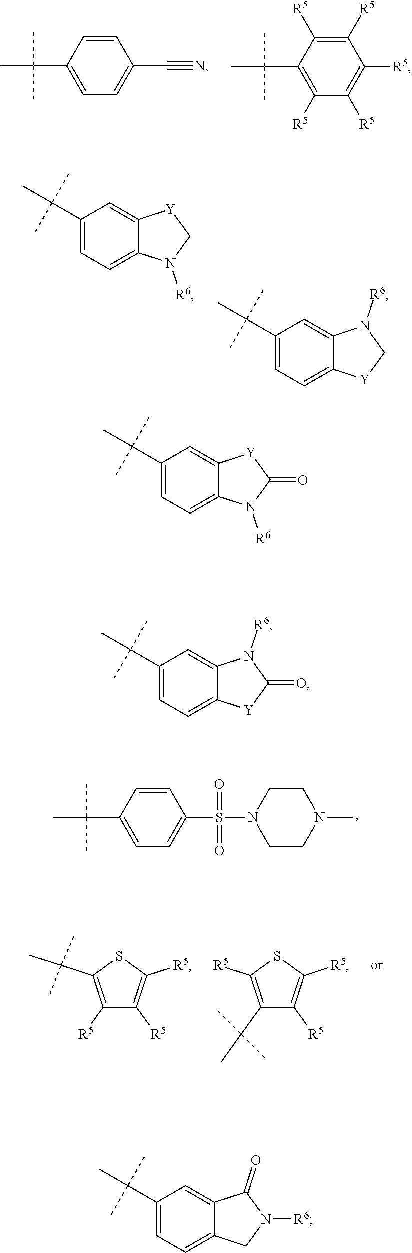

X₁, X₂ and X₃ are independently O, S, NR³ or CR³R⁴;
each R³, R⁴ and R⁵ is independently H, F, Cl, Br, I or C₁-C₆ alkyl;
each R⁶ is independently H or C₁-C₆ alkyl;
each Y is independently O, S, CHR⁶ or NR⁶; and
each m and m′ is independently an integer from 0-3, and the total sum of m and m′ is ≤3;
each n, n′ and n″ is independently an integer from 0-3, and the total sum of n, n′ and n″ is ≤4; and
L is

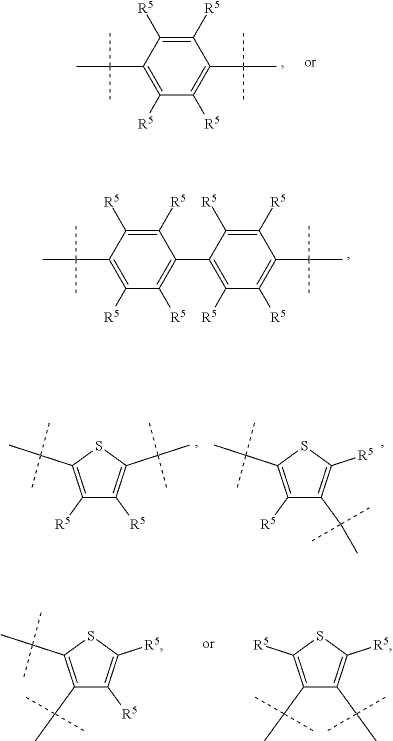

provided that,
   (i) when R¹ is

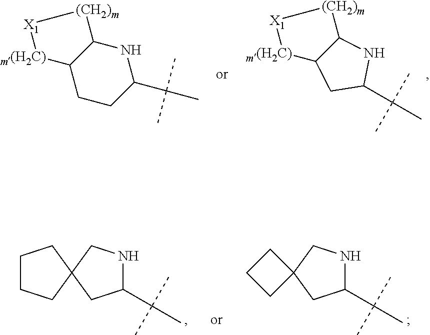

and X₁ is —CH₂—, then L and R² taken together, is not

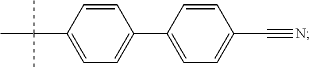

or
(ii) when R¹ is

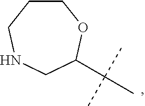

then L and R² taken together, is not

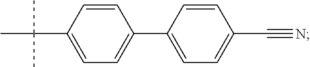

(iii) when R¹ is

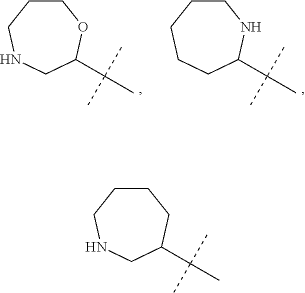

and L is

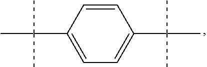

then R² is not

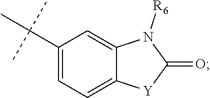

(iv) when R¹ is

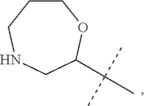

and L is

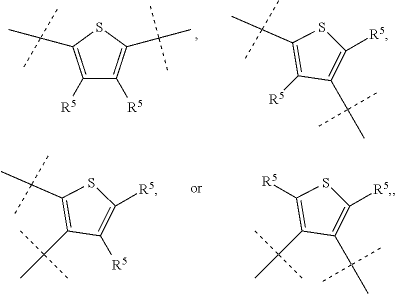

then R² is not

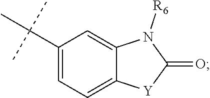

(v) when R¹ is

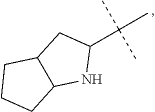

and L is

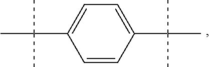

then R² is not

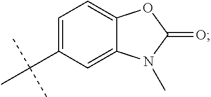

(vi) when R¹ is

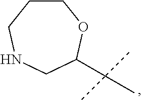

and L is

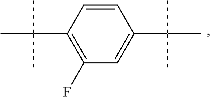

then R² is not

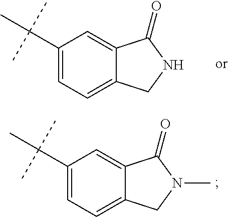

or
(vii) when R¹ is

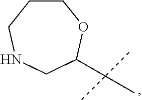

and L is

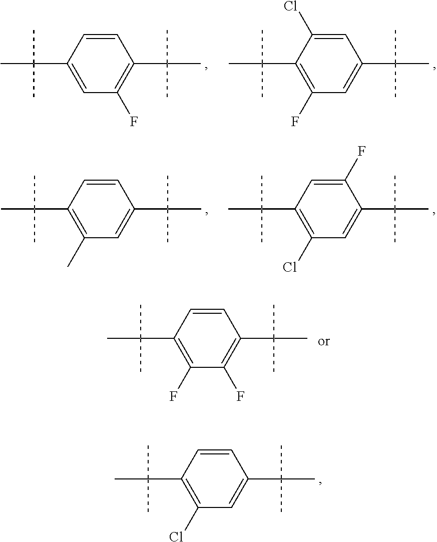

then R² is not

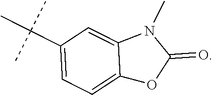

In another aspect of the invention, a DPP1 inhibitor of Formula (I-I), or a pharmaceutically acceptable or deuterated form thereof, is provided:

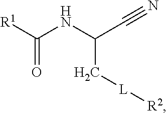

or a pharmaceutically acceptable salt or deuterated form thereof, wherein,
R¹ is

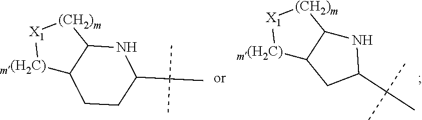

R² is

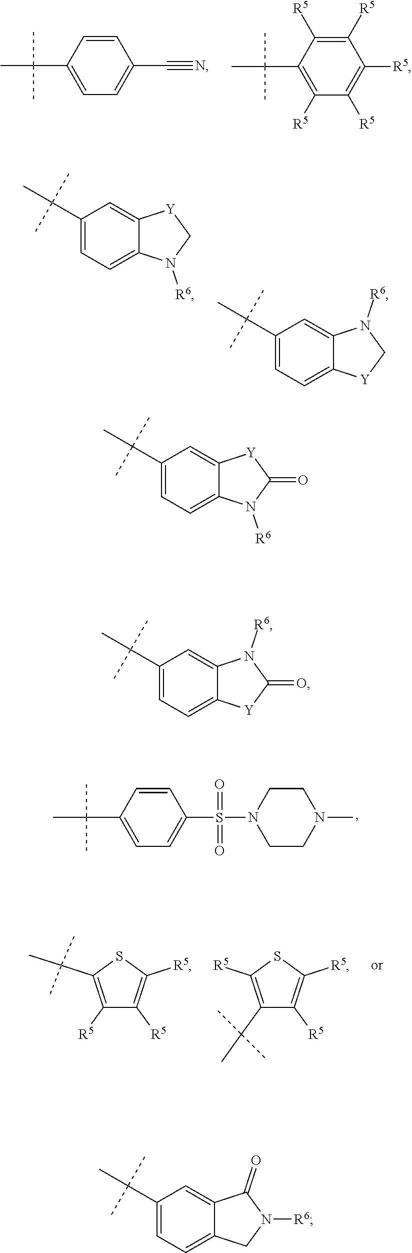

X₁, X₂ and X₃ are independently O, S, NR³ or CR³R⁴;
each R³, R⁴ and R⁵ is independently H, F, Cl, Br, I or C₁-C₆ alkyl;
each R⁶ is independently H or C₁-C₆ alkyl;
each Y is independently O, S, CHR⁶ or NR⁶;
m and m′ are each independently an integer from 0-3, and the total sum of m and m′ is ≤3; and

L is

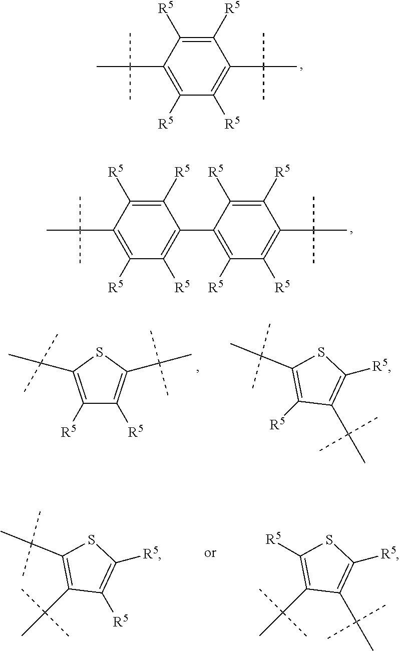

provided that, the compound is not:

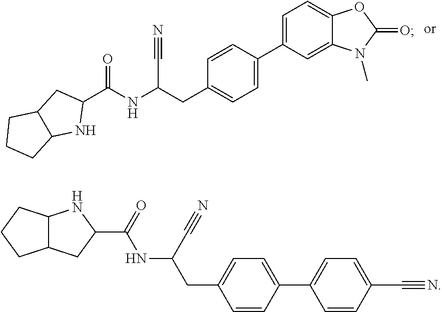 [SMILES: Cn1c(=O)oc2ccc(-c3ccc(CC(C#N)NC(=O)C4CC5CCCC5N4)cc3)cc21 ; "or" ; N#Cc1ccc(-c2ccc(CC(C#N)NC(=O)C3CC4CCCC4N3)cc2)cc1]

In another aspect of the invention, a DPP1 inhibitor of Formula (II), or a pharmaceutically acceptable salt thereof, is provided:

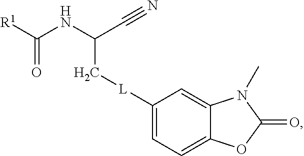

wherein,

R¹ is

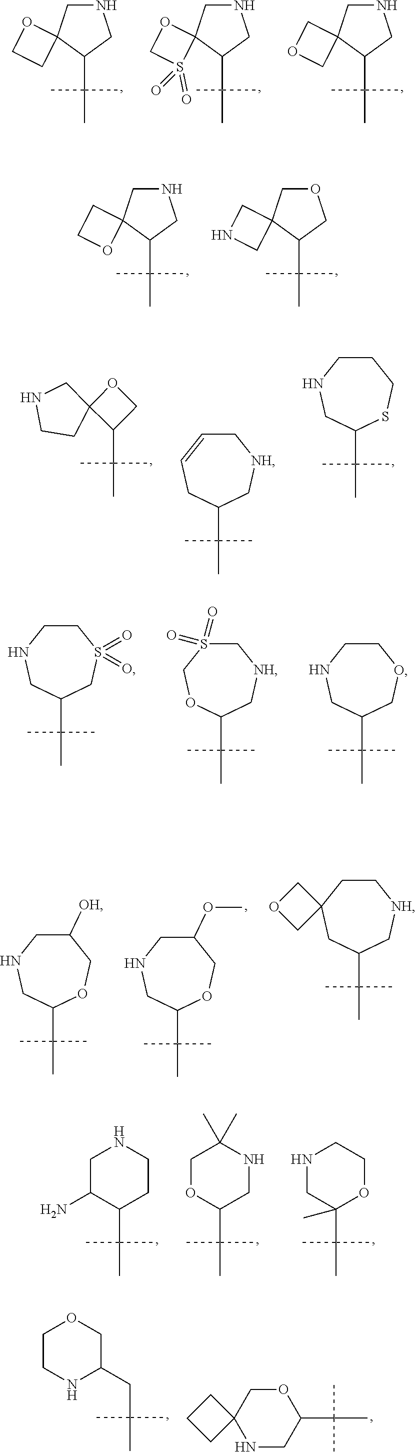

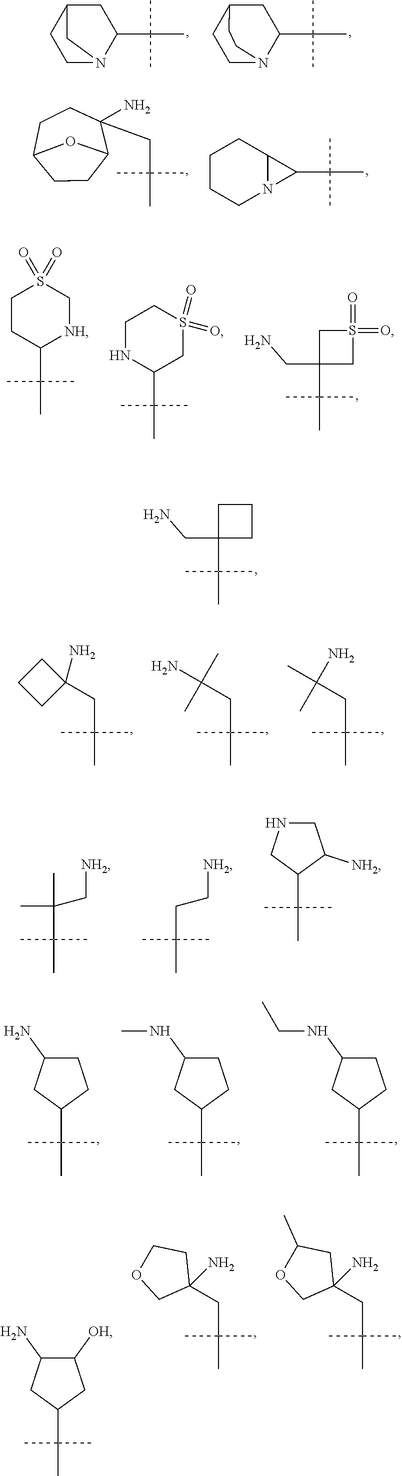

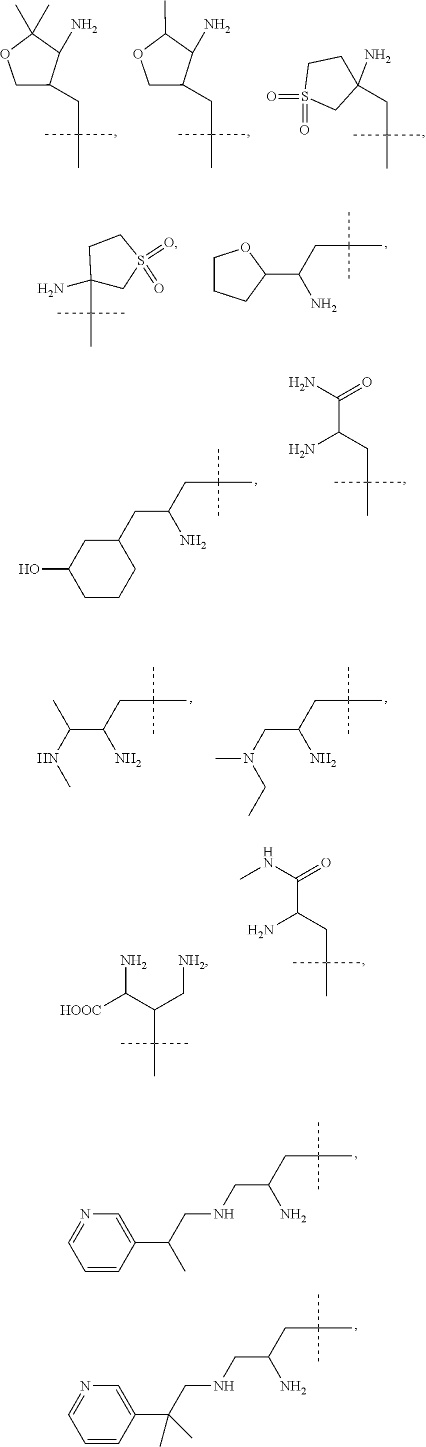

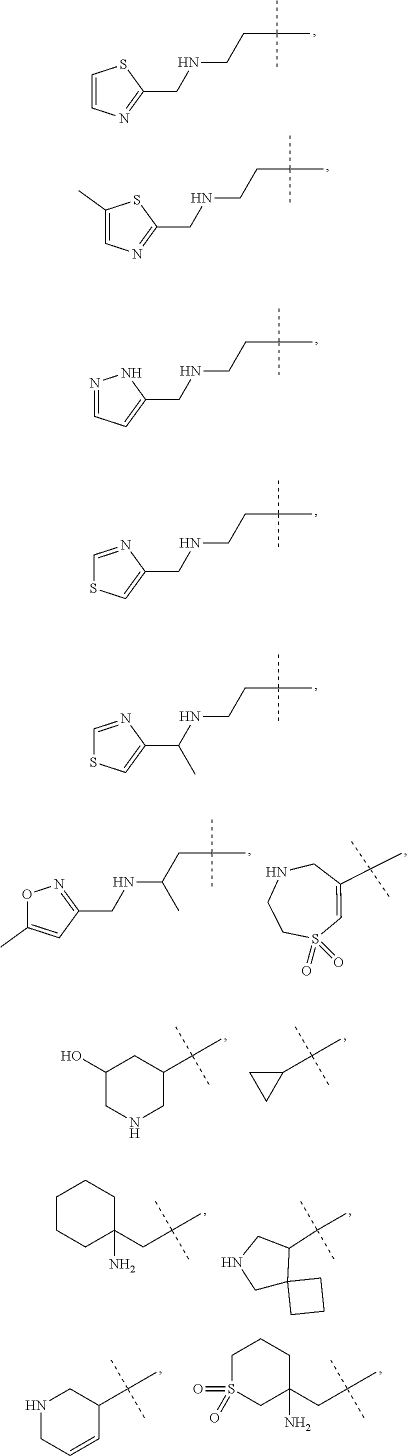

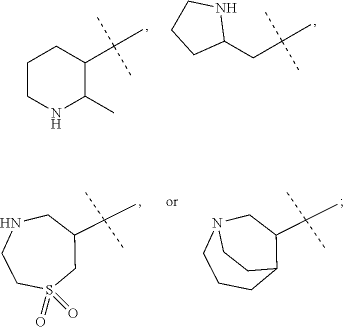

L is

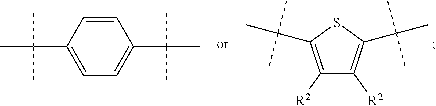

and
each R² is independently H, F, Cl, Br, I or C₁-C₆ alkyl, wherein the compound is not

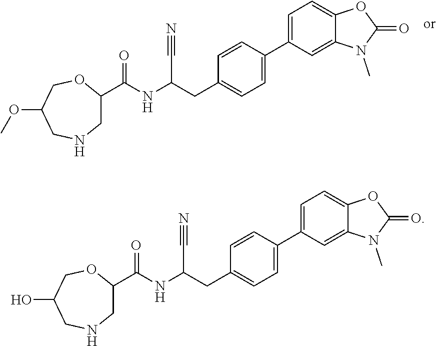 [SMILES: COC1CNCC(C(=O)NC(C#N)Cc2ccc(-c3ccc4oc(=O)n(C)c4c3)cc2)OC1 ; Cn1c(=O)oc2ccc(-c3ccc(CC(C#N)NC(=O)C4CNCC(O)CO4)cc3)cc21]

In one embodiment of a compound of Formula (II), or a pharmaceutically acceptable salt thereof, L is

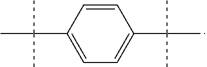

In even another aspect, a DPP1 inhibitor provided herein is a sulfonamide compound of Formula (III), or a pharmaceutically acceptable salt thereof:

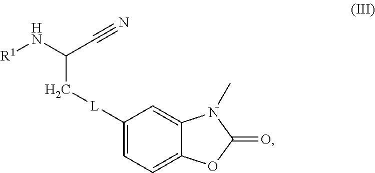

wherein,
R¹ is

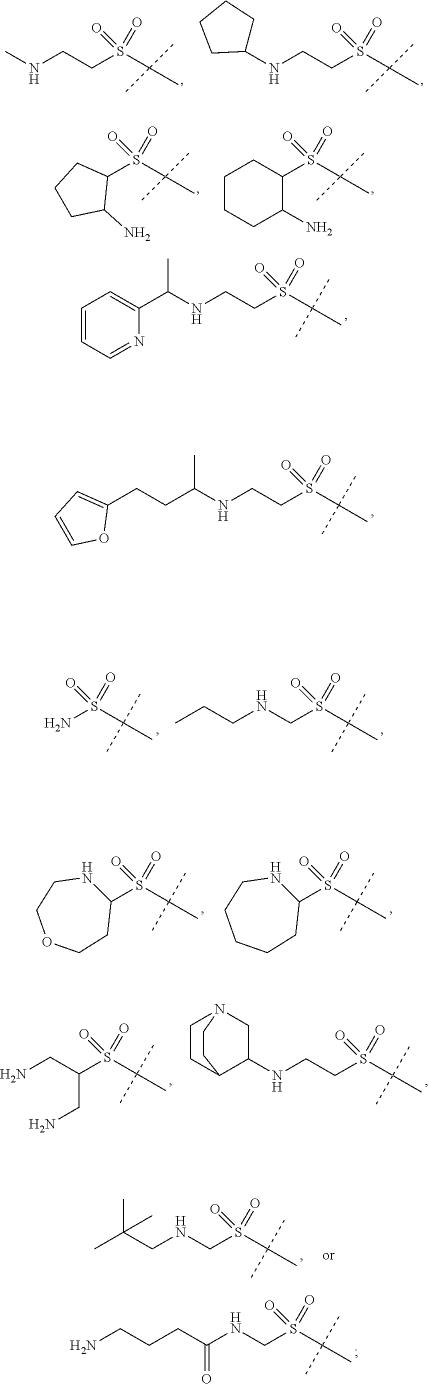

L is and

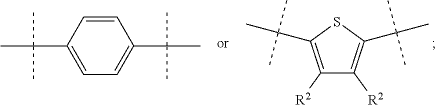

each R² is independently H, F, Cl, Br, I or C₁-C₆ alkyl.

In even another aspect, a DPP1 inhibitor provided herein is a sulfonamide compound of Formula (III), or a pharmaceutically acceptable salt thereof:

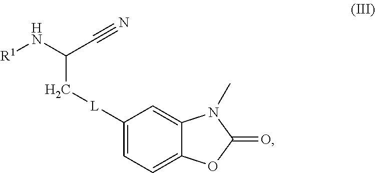

wherein,
R¹ is

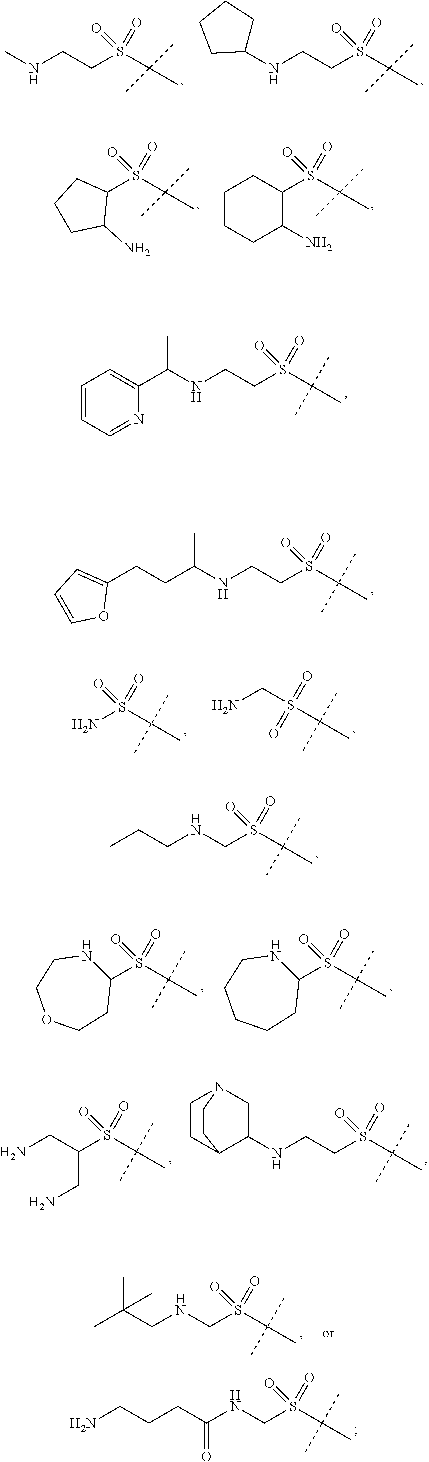

L is

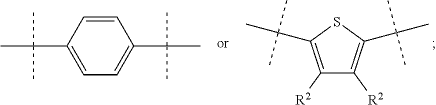

and
each R² is independently H, F, Cl, Br, I or C₁-C₆ alkyl.

In some embodiments of compound of Formula (III), R¹ is

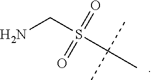

In yet another aspect of the invention, a method of treatment is provided. The method of treatment, in one embodiment, comprises, administering to a subject in need of, a composition comprising an effective amount of a compound of Formula (I), (II) or (III), or a pharmaceutically acceptable salt of Formula (I), (II) or (III). In one embodiment of the method, the composition is administered orally. In a further embodiment, the composition is administered orally, once daily.

The method of treatment, in one embodiment, is a method of treating an obstructive disease of the airway, e.g., cystic fibrosis (CF), asthma or bronchiectasis (e.g., non-CF bronchiectasis).

In another embodiment, the method of treatment is a method of treating chronic rhinosinusitis (CRS). In yet another embodiment, the method of treatment is a method of treating

### BRIEF DESCRIPTION OF THE DRAWINGS

FIG. 1 shows the chemical synthesis scheme for one compound of the invention, IWD-002 ((1R,3S,5R)—N—((S)-1-cyano-2-(4-(3-methyl-2-oxo-2,3-dihydrobenzo[d]oxazol-5-yl)phenyl)ethyl)-2-azabicyclo[3.1.0]hexane-3-carboxamide).

FIG. 2 shows the chemical synthesis scheme for one compound of the invention, IWD-004 (2S,3αR,6αS)—N—((S)-1-cyano-2-(4′-cyano-[1,1′-biphenyl]-4-yl)ethyl)hexahydro-1H-furo[3,4-b]pyrrole-2-carboxamide).

FIG. 3 shows the chemical synthesis scheme for one compound of the invention, IWD-005 ((2S,3αR,6αS)—N—((S)-1-cyano-2-(4-(3-methyl-2-oxo-2,3-dihydrobenzo[d]oxazol-5-yl)phenyl)ethyl)hexahydro-1H-furo[3,4-b]pyrrole-2-carboxamide).

FIG. 4 shows a chemical synthesis scheme for a reactant, (S)-2-amino-3-(4-(3-methyl-2-oxo-2,3-dihydrobenzo[d]oxazol-5-yl)phenyl)propanenitrile hydrochloride, that can be used to generate a compound of Formula (IA) or (II).

FIG. 5 shows a chemical synthesis scheme for an exemplary cyclic bridged α-amino moiety, which can be used as a reactant to generate a compound of Formula (II).

FIG. 6 shows a chemical synthesis scheme for an exemplary cyclic sulfonyl α-amino moiety, which can be used as a reactant to generate a compound of Formula (II).

FIG. 7 shows a chemical synthesis scheme for an exemplary cyclic sulfonyl α-amino moiety, which can be used as a reactant to generate a compound of Formula (II).

FIG. 8 shows a chemical synthesis scheme for an exemplary exo-beta NH₂ cyclic moiety, which can be used as a reactant for obtaining a compound of the invention.

FIG. 9 shows a chemical synthesis scheme for an exemplary beta-amino spirocyclic moiety, which can be used as a reactant for obtaining a compound of the invention.

FIG. 10 shows the chemical synthesis scheme for one DPP1 inhibitor of the invention, B1-26.

FIG. 11 shows the chemical synthesis scheme for one DPP1 inhibitor of the invention, B1-18.

FIG. 12 are graphs showing in vitro inhibition of mouse DPP1 as a function of the concentration of certain DPP1 inhibitors of the invention (IWD-004—left; IWD-005, right). The dashed lines indicate the IC50 for each compound (IWD-004:209 nM; IWD-005:45.6 nM).

FIG. 13 is a graph showing in vitro inhibition of mouse DPP1 as a function of brensocatib concentration. The dashed line indicates the IC50 for brensocatib (24.3 nM).

FIG. 14 are graphs showing in vitro inhibition of human DPP1 as a function of the concentration (nM) of certain DPP1 inhibitors of the invention (IWD-004—left; IWD-005, right). The dashed lines indicate the IC50 for each compound (IWD-004:245 nM; IWD-005: 69.2 nM).

FIG. 15 is a graph showing in vitro inhibition of human DPP1 as a function of brensocatib concentration (nM). The dashed line indicates the IC50 for brensocatib (16.0 nM).

FIG. 16 are graphs showing the percent DPP1 inhibition in HL-60 cells using H-Gly-Phe-AFC substrate, as a function of brensocatib concentration (nM, left) or IWD-002 concentration (nM, right).

FIG. 17 is a graph showing the percent DPP1 inhibition in HL-60 cells using H-Gly-Phe-AFC substrate, as a function of GSK-2793660 concentration (nM).

### DETAILED DESCRIPTION OF THE INVENTION

The term “alkyl”, unless otherwise noted, includes both straight and branched chain alkyl groups and may be, substituted or non-substituted. “Alkyl” groups include, but are not limited to, methyl, ethyl, n-propyl, i-propyl, butyl, pentyl.

The term “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

### Formula (I)

In one aspect of the present invention, a DPP1 inhibitor compound is provided. In one embodiment, of this aspect, a compound of Formula (I), or a pharmaceutically acceptable salt or deuterated form thereof, is provided:

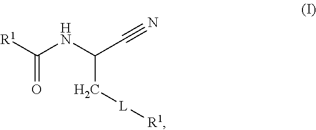

wherein,
R¹ is

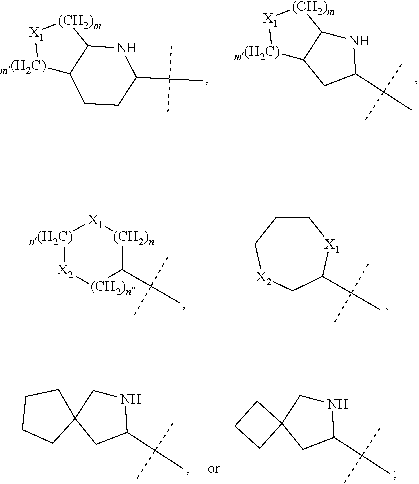

R² is

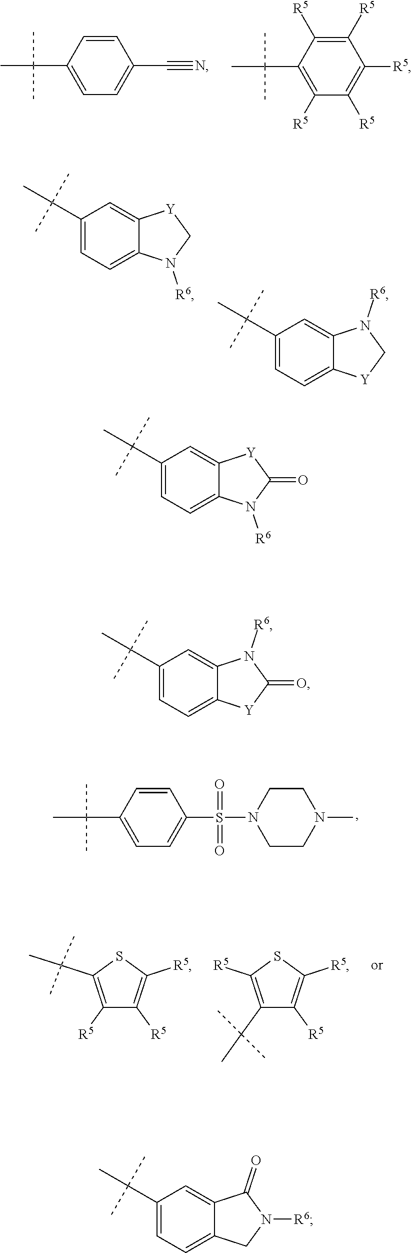

X₁, X₂ and X₃ are independently O, S, NR³ or CR³R⁴;
each R³, R⁴ and R⁵ is independently H, F, Cl, Br, I or C₁-C₆ alkyl;
each R⁶ is independently H or C₁-C₆ alkyl;
each Y is independently O, S, CHR⁶ or NR⁶; and
each m and m′ is independently an integer from 0-3, and the total sum of m and m′ is ≤3;
each n, n′ and n″ is independently an integer from 0-3, and the total sum of n, n′ and n″ is ≤4; and
L is

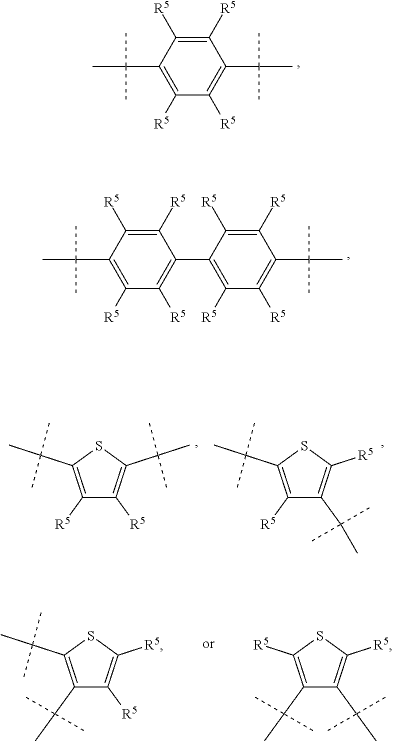

provided that,
   (i) when R¹ is

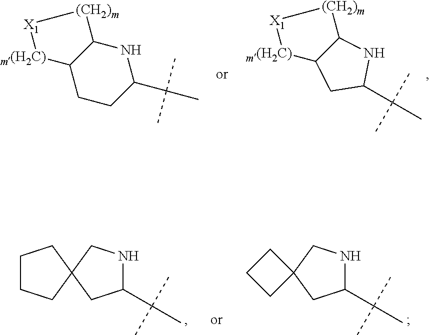

and X₁ is —CH₂—, then L and R² taken together, is not

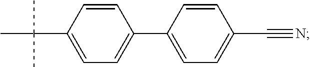

or
(ii) when R¹ is

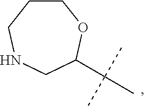

then L and R² taken together, is not

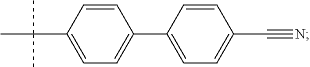

(iii) when R¹ is

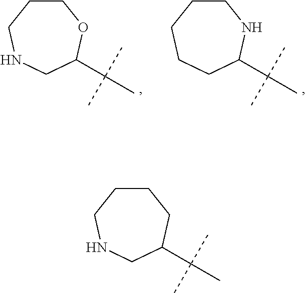

and L is

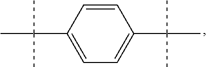

then R² is not

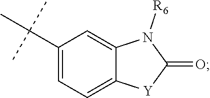

(iv) when R¹ is

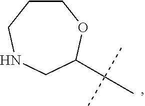

and L is

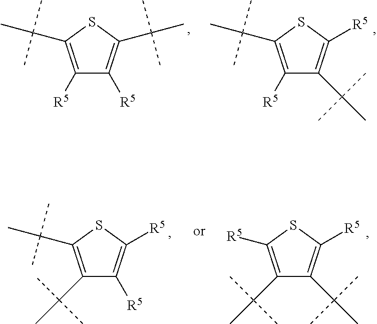

then R² is not

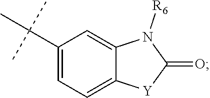

(v) when R¹ is

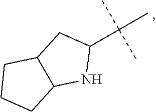

and L is

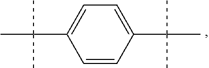

then R² is not

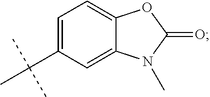

(vi) when R¹ is

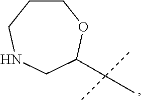

and L is

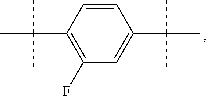

then R² is not

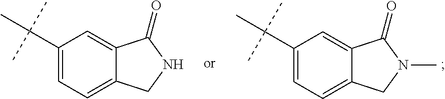

or
(vii) when R¹ is

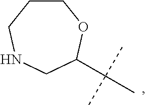

and L is

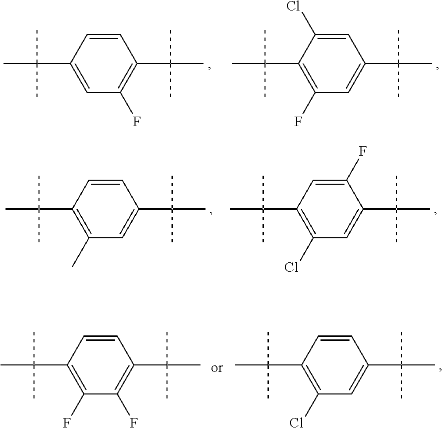

then R² is not

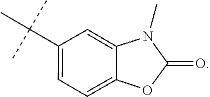

In one embodiment of a compound of Formula (I), or a pharmaceutically acceptable salt or deuterated form thereof, R¹ is

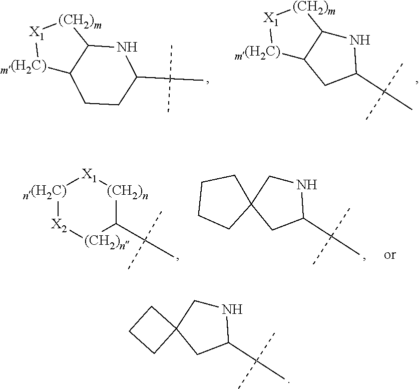

In one embodiment of a compound of Formula (I), or a pharmaceutically acceptable salt or deuterated form thereof, R¹ is

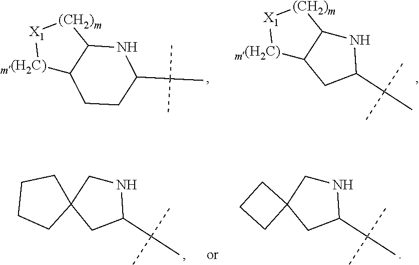

In another embodiment of a compound of Formula (I), R¹ is

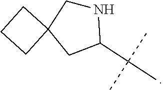

In even another embodiment of a compound of Formula (I), R¹ is

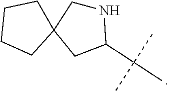

In yet even another embodiment of a compound of Formula (I), R¹ is

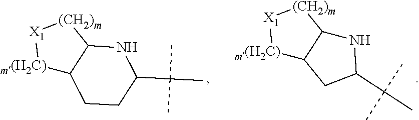

In yet even another embodiment of a compound of Formula (I), R¹ is

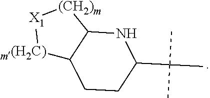

In yet even another embodiment of a compound of Formula (I), R¹ is

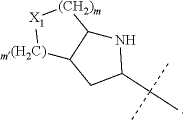

Yet another embodiment of a compound of Formula (I) is provided, where the compound of Formula (I) is a compound of Formulae (IA), (IB), (IC), (ID), or a pharmaceutically acceptable salt or deuterated form of one of the foregoing. For compounds of Formulae (IA), (IB), (IC), (ID), or a pharmaceutically acceptable salt or deuterated form thereof, R¹ is defined as it is in Formula (I).

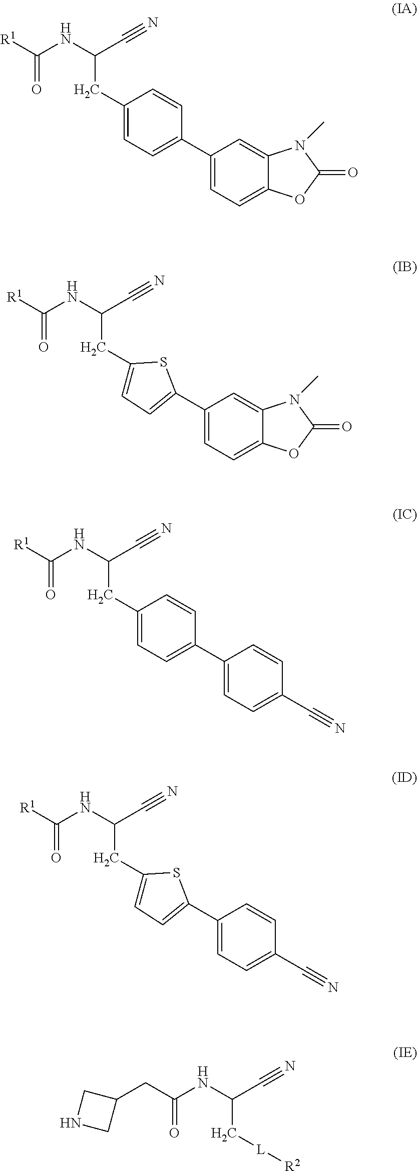

In one embodiment of a compound of Formula (I) is provided, wherein the compound of Formula (I) is a compound of Formula (I-I)

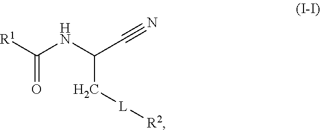

or a pharmaceutically acceptable salt or deuterated form thereof, wherein,
R¹ is

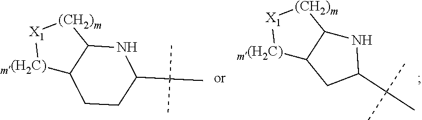

R² is

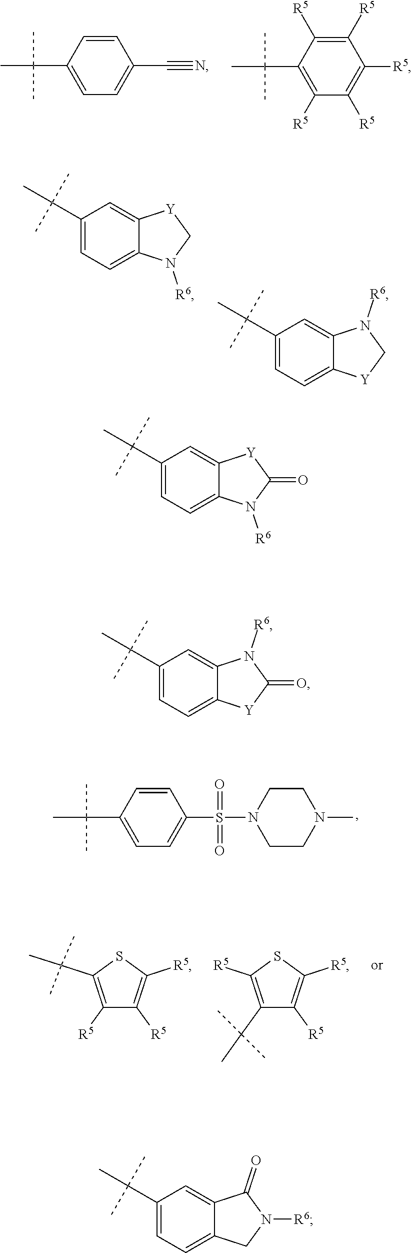

X₁, X₂ and X₃ are independently O, S, NR³ or CR³R⁴;
each R³, R⁴ and R⁵ is independently H, F, Cl, Br, I or C₁-C₆ alkyl;
each R⁶ is independently H or C₁-C₆ alkyl;
each Y is independently O, S, CHR⁶ or NR⁶;
m and m′ are each independently an integer from 0-3, and the total sum of m and m′ is ≤3; and
L is

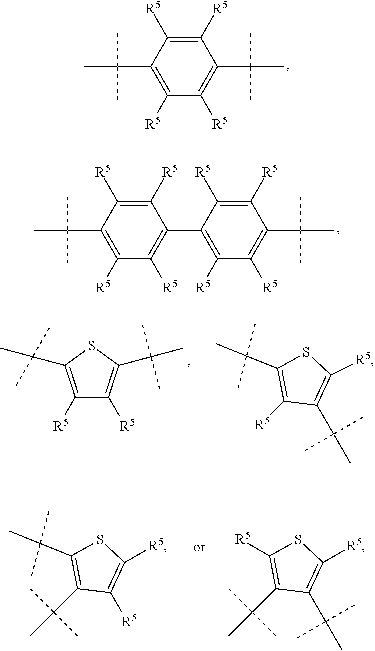

provided that, the compound is not:

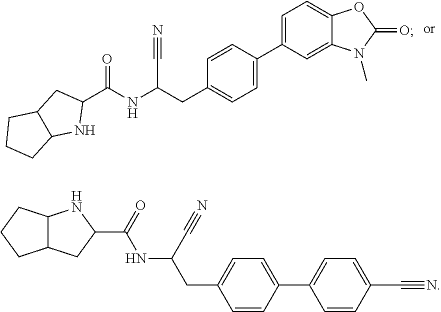 [SMILES: Cn1c(=O)oc2ccc(-c3ccc(CC(C#N)NC(=O)C4CC5CCCC5N4)cc3)cc21 ; "or" ; N#Cc1ccc(-c2ccc(CC(C#N)NC(=O)C3CC4CCCC4N3)cc2)cc1]

Yet another embodiment of a compound of Formula (I-I) is provided, where the compound of Formula (I-I) is a compound of Formulae (IA-I), (IB-I), (IC-I), (ID-I), or a pharmaceutically acceptable salt or deuterated form of one of the foregoing. For compounds of Formulae (IA-I), (IB-I), (IC-I), (ID-I), or a pharmaceutically acceptable salt or deuterated form thereof, m′, m, L, X₁, R², and R⁵ are defined as in Formula (I-I).

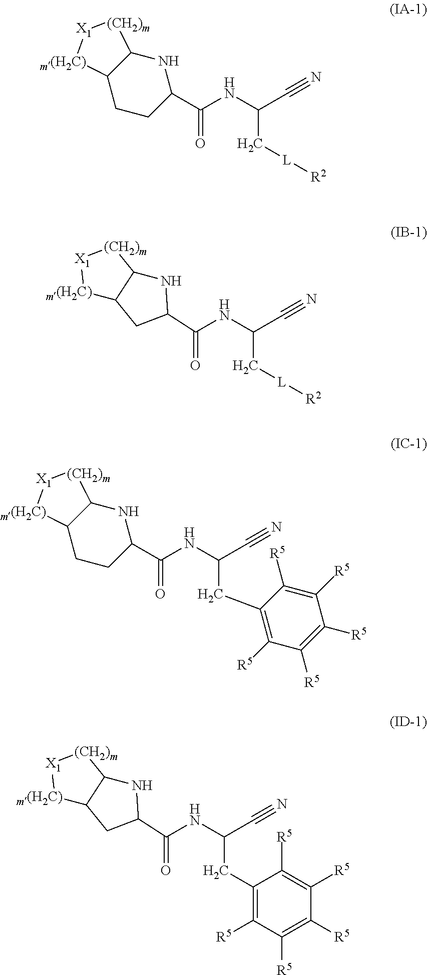

In one embodiment of Formula (I) (or a pharmaceutic ally acceptable salt or deuterated form thereof), where R¹ is

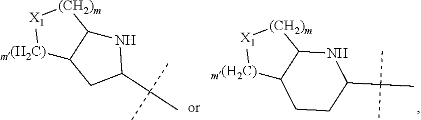

e.g., a compound of Formula (I-I), (IA), (IB), (IC), (ID), (IE), (I-I), (IA-I), (IB-I), (IC-I), (ID-I), or a pharmaceutically acceptable salt or deuterated form of one of the foregoing, m and m′ have one of the combination of values set forth in Table 1.

TABLE 1

| Compound | R¹ | m | m′ | X₁ |
|---|---|---|---|---|
| 1. | 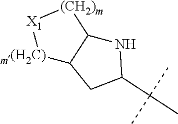 | 0 | 3 | O |
| 2. | 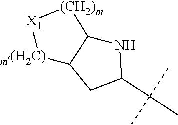 | 0 | 2 | O |
| 3. | 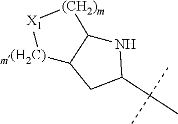 | 0 | 1 | O |
| 4. | 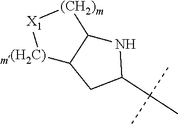 | 0 | 0 | O |
| 5. | 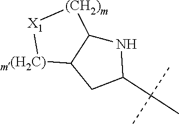 | 1 | 2 | O |
| 6. | 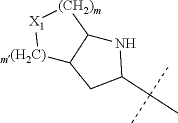 | 1 | 1 | O |
| 7. | 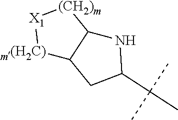 | 1 | 0 | O |
| 8. | 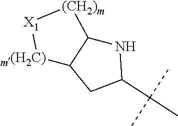 | 2 | 1 | O |
| 9. | 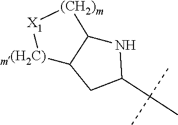 | 2 | 0 | O |
| 10. | 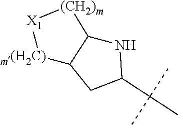 | 3 | 0 | O |
| 11. | 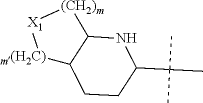 | 0 | 3 | O |
| 12. | 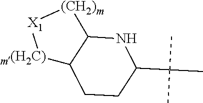 | 0 | 2 | O |
| 13. | 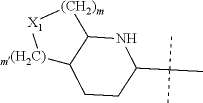 | 0 | 1 | O |
| 14. | 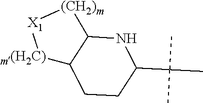 | 0 | 0 | O |
| 15. | 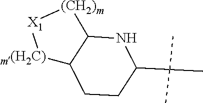 | 1 | 2 | O |
| 16. | 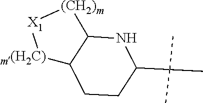 | 1 | 1 | O |
| 17. | 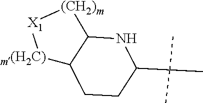 | 1 | 0 | O |
| 18. | 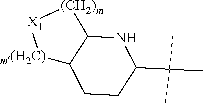 | 2 | 1 | O |
| 19. | 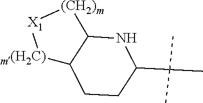 | 2 | 0 | O |
| 20. | 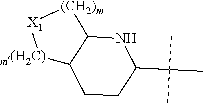 | 3 | 0 | O |
| 21. | 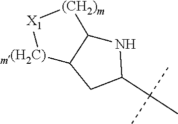 | 0 | 3 | S |
| 22. | 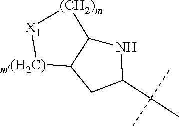 | 0 | 2 | S |
| 23. | 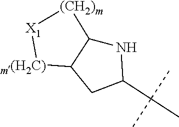 | 0 | 1 | S |
| 24 | 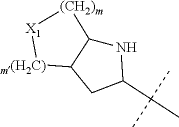 | 0 | 0 | S |
| 25. | 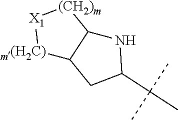 | 1 | 2 | S |
| 26. | 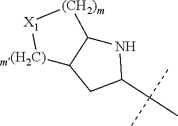 | 1 | 1 | S |
| 27. | 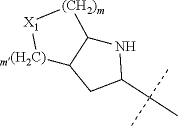 | 1 | 0 | S |
| 28. | 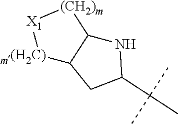 | 2 | 1 | S |
| 29 | 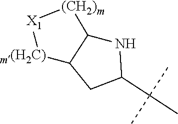 | 2 | 0 | S |
| 30. | 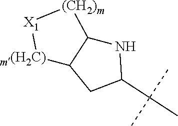 | 3 | 0 | S |
| 31. | 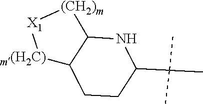 | 0 | 3 | S |
| 32. | 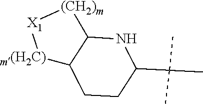 | 0 | 2 | S |
| 33. | 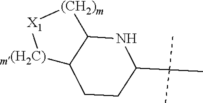 | 0 | 1 | S |
| 34. | 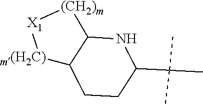 | 0 | 0 | S |
| 35. | 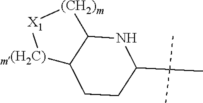 | 1 | 2 | S |
| 36 | 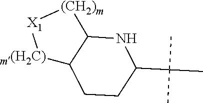 | 1 | 1 | S |
| 37. | 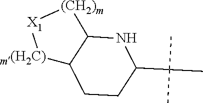 | 1 | 0 | S |
| 38 | 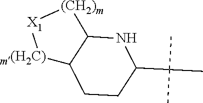 | 2 | 1 | S |
| 39. | 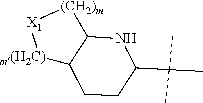 | 2 | 0 | S |
| 40. | 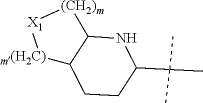 | 3 | 0 | S |
| 41. | 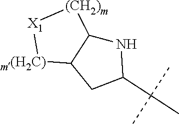 | 0 | 3 | NH |
| 42. | 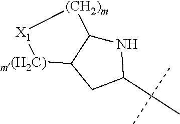 | 0 | 2 | NH |
| 43. | 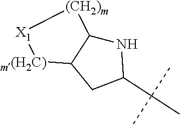 | 0 | 1 | NH |
| 44. | 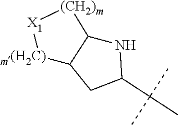 | 0 | 0 | NH |
| 45. | 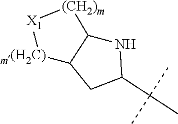 | 1 | 2 | NH |
| 46. | 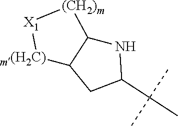 | 1 | 1 | NH |
| 47. | 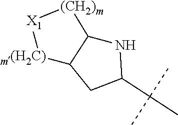 | 1 | 0 | NH |
| 48. | 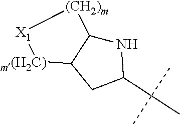 | 2 | 1 | NH |
| 49. | 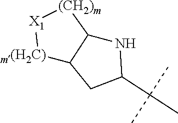 | 2 | 0 | NH |
| 50. | 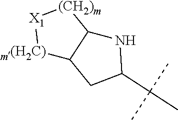 | 3 | 0 | NH |
| 51. | 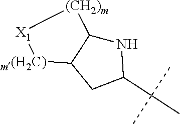 | 0 | 3 | NH |
| 52. | 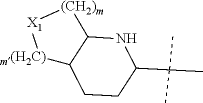 | 0 | 2 | NH |
| 53. | 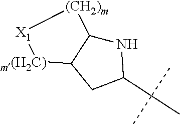 | 0 | 1 | NH |
| 54. | 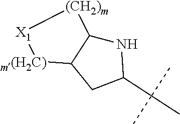 | 0 | 0 | NH |
| 55. | 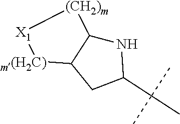 | 1 | 2 | NH |
| 56. | 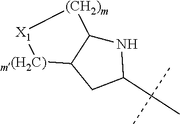 | 1 | 1 | NH |
| 57. | 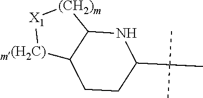 | 1 | 0 | NH |
| 58. | 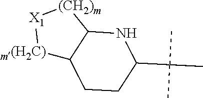 | 2 | 1 | NH |
| 59. | 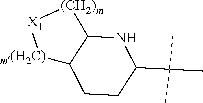 | 2 | 0 | NH |
| 60. | 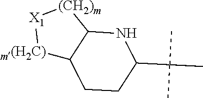 | 3 | 0 | NH |
| 61. | 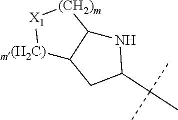 | 0 | 3 | CH₂ |
| 62. | 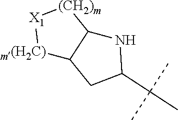 | 0 | 2 | CH₂ |
| 63. | 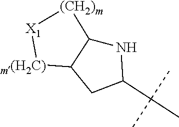 | 0 | 1 | CH₂ |
| 64 | 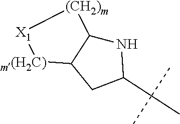 | 0 | 0 | CH₂ |
| 65. | 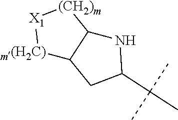 | 1 | 2 | CH₂ |
| 66 | 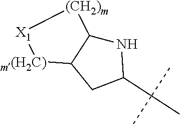 | 1 | 1 | CH₂ |
| 67. | 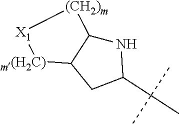 | 1 | 0 | CH₂ |
| 68. | 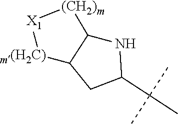 | 2 | 1 | CH₂ |
| 69. | 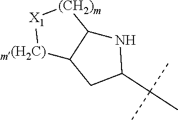 | 2 | 0 | CH₂ |
| 70. | 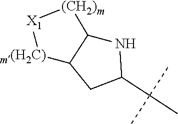 | 3 | 0 | CH₂ |
| 71. | 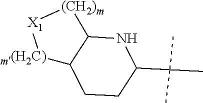 | 0 | 3 | CH₂ |
| 72. | 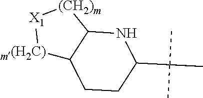 | 0 | 2 | CH₂ |
| 73. | 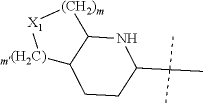 | 0 | 1 | CH₂ |
| 74. | 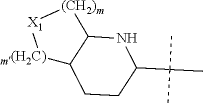 | 0 | 0 | CH₂ |
| 75. | 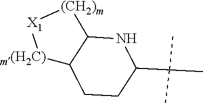 | 1 | 2 | CH₂ |
| 76. | 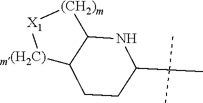 | 1 | 1 | CH₂ |
| 77. | 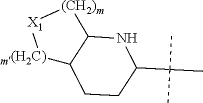 | 1 | 0 | CH₂ |
| 78. | 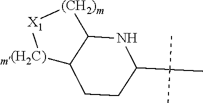 | 2 | 1 | CH₂ |
| 79. | 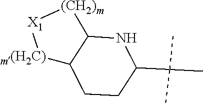 | 2 | 0 | CH₂ |
| 80. | 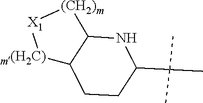 | 3 | 0 | CH₂ |

In one embodiment, R¹ is

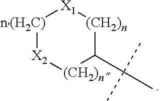

In a further embodiment, the sum of n, n′ and n″ is 3.

In one embodiment, R¹ is

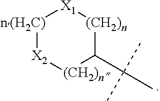

In a further embodiment, the sum of n, n′ and n″ is 2.

In one embodiment, R¹ is

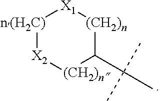

In a further embodiment, the sum of n, n′ and n″ is 1.

In one embodiment of Formula (I), R¹ is

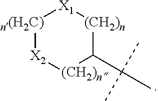

In a further embodiment, the sum of n, n′ and n″ is 0.

In another embodiment of Formula (I), R¹ is

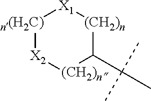

and the values of n, n′ and n″ are selected from one of the combinations set forth in Table 2.

TABLE 2

| Sum of n, | | | |
|---|---|---|---|
| n′ and n″ | n | n′ | n″ |
| 0 | 0 | 0 | 0 |
| 1 | 0 | 1 | 0 |
| | 0 | 0 | 1 |
| | 1 | 0 | 0 |
| 2 | 2 | 0 | 0 |
| | 0 | 2 | 0 |
| | 0 | 0 | 2 |
| | 1 | 1 | 0 |
| | 1 | 0 | 1 |
| | 0 | 1 | 1 |
| 3 | 0 | 0 | 3 |
| | 0 | 3 | 0 |
| | 3 | 0 | 0 |
| | 0 | 1 | 2 |
| | 0 | 2 | 1 |
| | 2 | 1 | 0 |
| | 2 | 0 | 1 |

In some embodiments of Formula (I), X₁ is O.

In some embodiments of Formula (I), X₁ is S.

In some embodiments of Formula (I), X₁ is NH.

In some embodiments of Formula (I), X₁ is CH₂.

In some embodiments of Formula (I), R² is

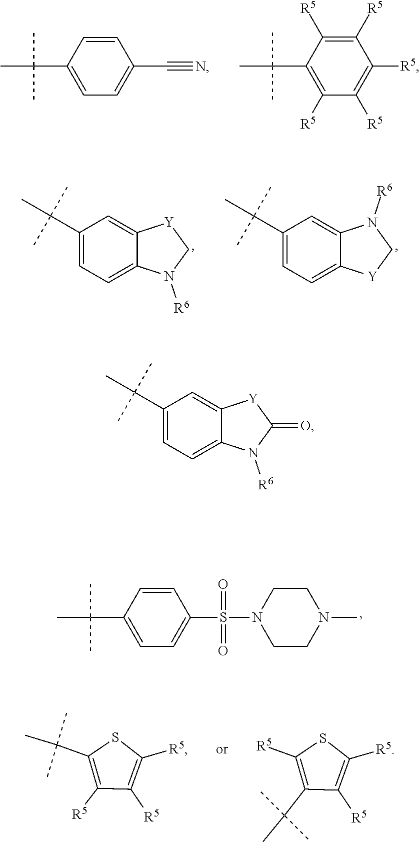

In one embodiment of Formula (I), R² is

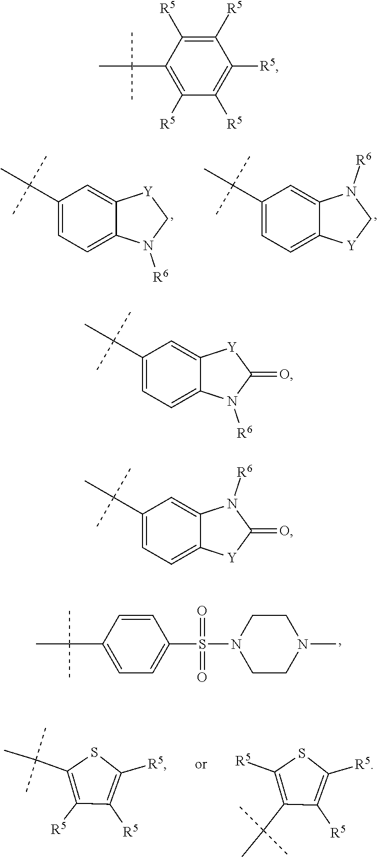

In some embodiments of Formula (I), R² is

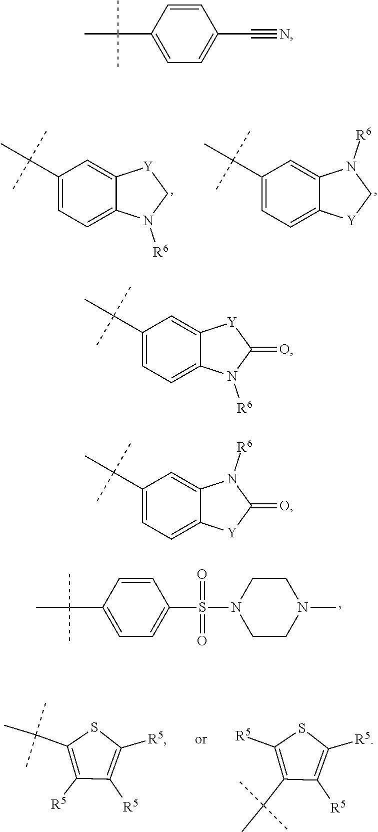

In another embodiment of Formula (I), R² is

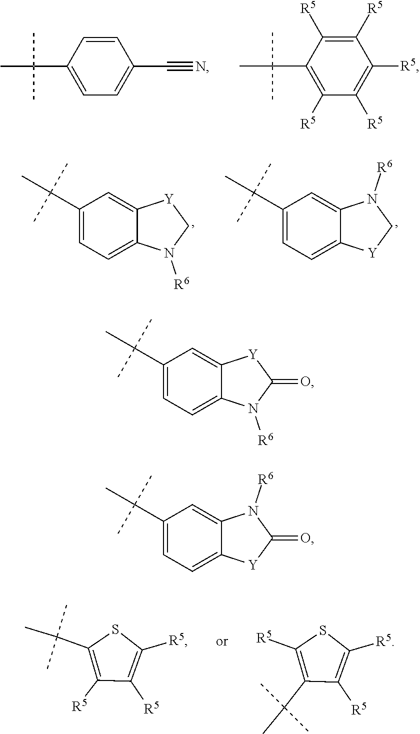

In some embodiments of Formula (I), R² is

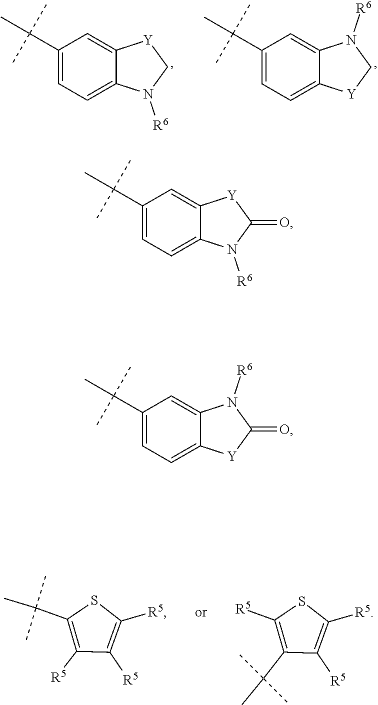

In yet another embodiment of Formula (I), R² is

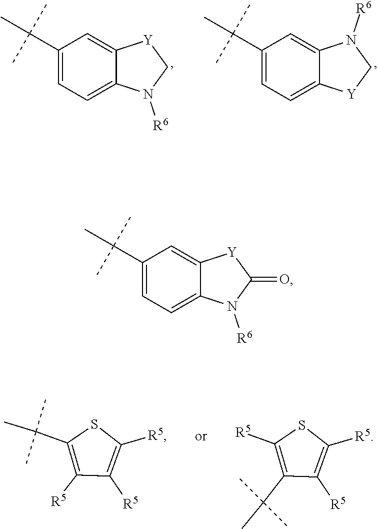

In embodiments of Formula (I), R² is

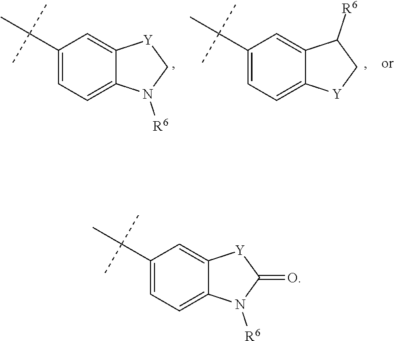

In some embodiments of Formula (I), R² is

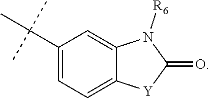

In some embodiments, R⁶ is H. In some embodiments, R⁶ is C₁-C₆ alkyl. In some embodiments, R⁶ is —CH₃. In some embodiments, Y is O. In some embodiments, Y is CH₂.

In another embodiment of Formula (I), R² is

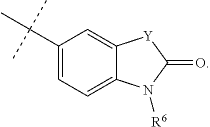

In some embodiments, R⁶ is H. In some embodiments, R⁶ is C₁-C₆ alkyl. In some embodiments, R⁶ is —CH₃. In some embodiments, Y is O. In some embodiments, Y is CH₂.

In another embodiment of Formula (I), R² is

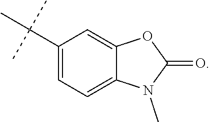

In some embodiments of Formula (I), R² is

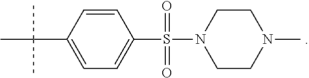

In yet another embodiment of Formula (I), R² is

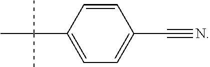

In another embodiment of Formula (I), R² is

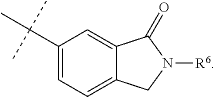

In some embodiments, R⁶ is H. In some embodiments, R⁶ is C₁-C₆ alkyl. In some embodiments, R⁶ is —CH₃.

In some embodiments of Formula (I), L is

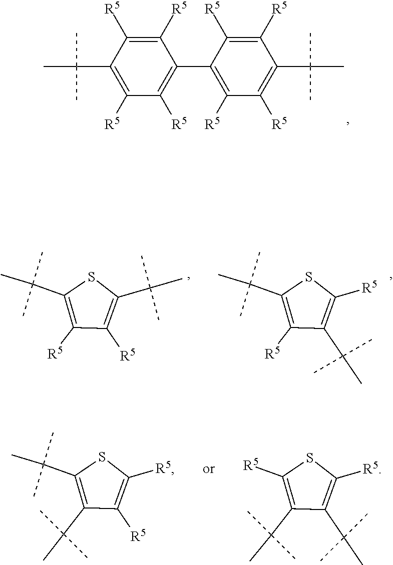

In some embodiments of Formula (I), L is

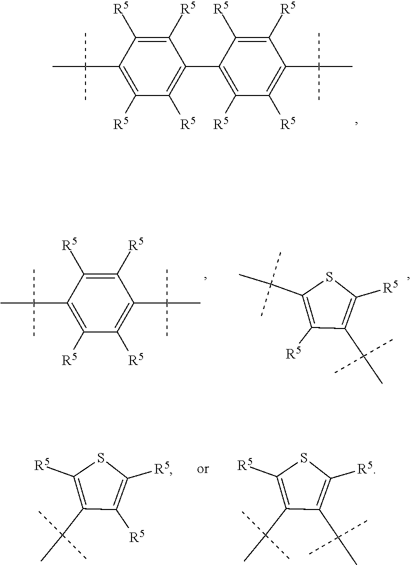

In another embodiment of Formula (I), L is

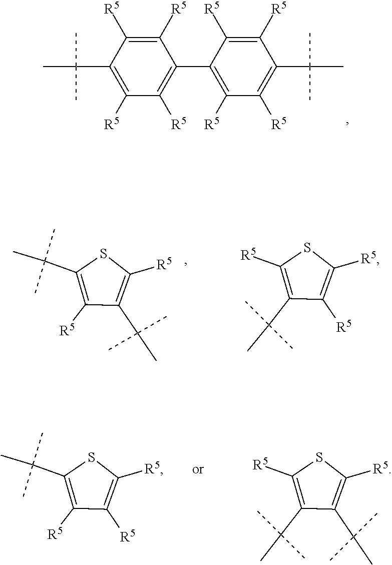

In yet another embodiment of Formula (I), L is

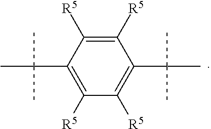

In some embodiments, each R⁵ is H. In some embodiments, at least one R⁵ is a halogen (e.g., F, Cl, Br, or I). In some embodiments, at least one R⁵ is a F. In some embodiments, at least two R⁵ is a F.

In some embodiments of Formula (I), L is

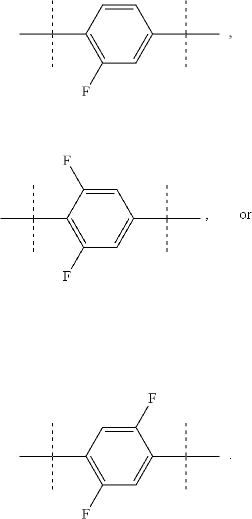

In some embodiments of Formula (I), L is

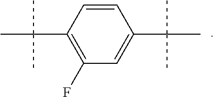

In some embodiments of Formula (I), L is

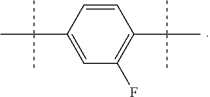

In some embodiments of Formula (I), L is

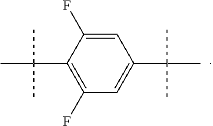

In some embodiments of Formula (I), L is

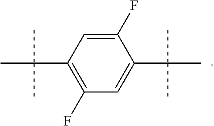

In some embodiments of Formula (I), L is

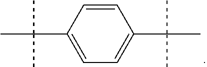

In other embodiments of Formula (I), L is

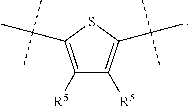

In some embodiments of Formula (I), L is

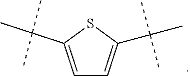

In some embodiments of Formula (I), L is

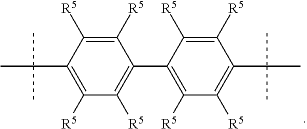

In some embodiments of Formula (I), L is

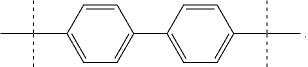

Other exemplary embodiments of compounds of Formula (I) are provided in Table 3. Note that these compounds, like the others disclosed herein, can also be provided as pharmaceutically acceptable salts or deuterated forms.

TABLE 3
Representative compounds of Formula (I).

| Compound | R¹ | R² | L |
|---|---|---|---|
| 81. | 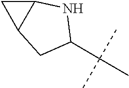 | 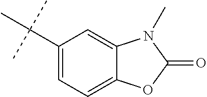 | 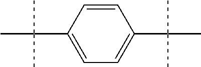 |
| 82. | 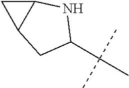 | 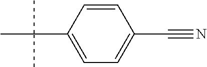 | 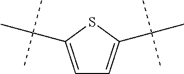 |
| 83. | 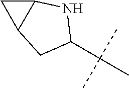 | 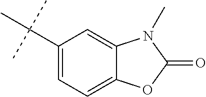 | 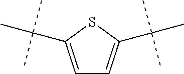 |
| 84. | 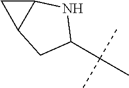 | 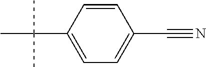 | 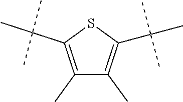 |
| 85. | 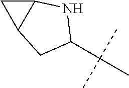 | 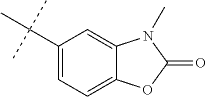 | 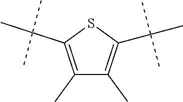 |
| 86. | 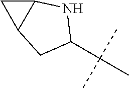 | 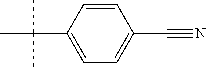 | 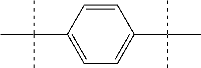 |
| 87. | 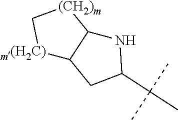 | 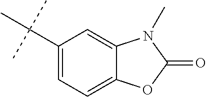 | 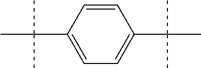 |
| 88. | 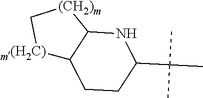 | 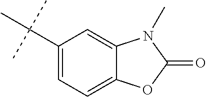 | 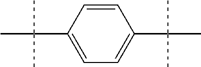 |
| 89. | 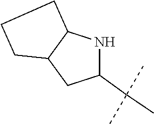 | 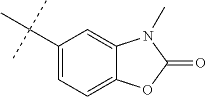 | 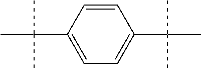 |
| 90. | 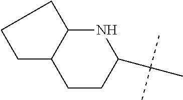 | 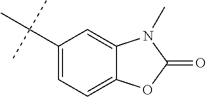 | 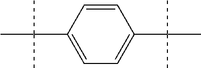 |
| 91. | 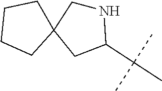 | 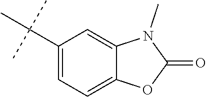 | 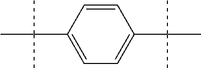 |
| 92. | 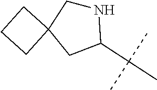 | 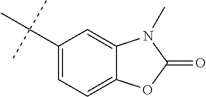 | 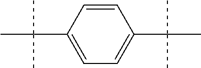 |
| 93. | 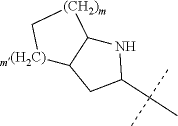 | 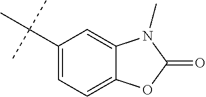 | 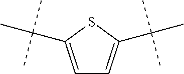 |
| 94. | 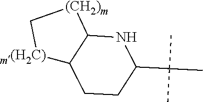 | 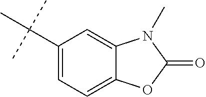 | 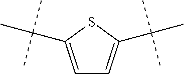 |
| 95. | 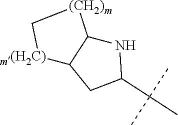 | 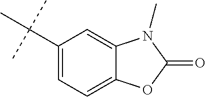 | 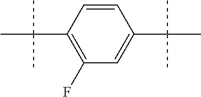 |
| 96. | 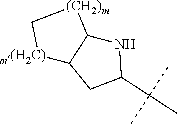 | 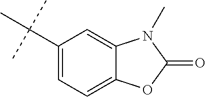 | 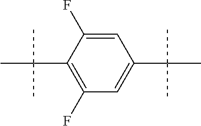 |
| 97. | 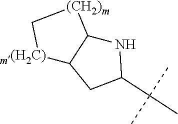 | 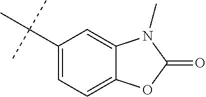 | 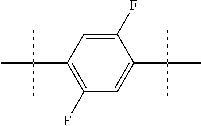 |
| 98. | 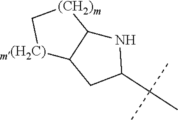 | 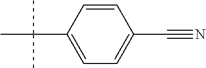 | 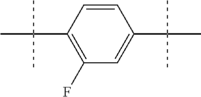 |
| 99. | 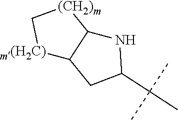 | 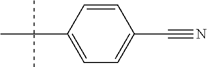 | 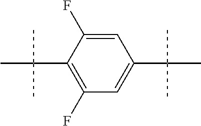 |
| 100. | 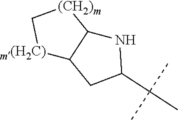 | 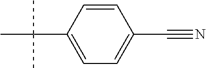 | 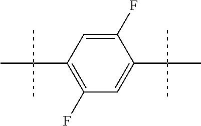 |
| 101. | 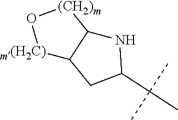 | 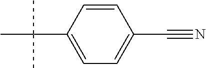 | 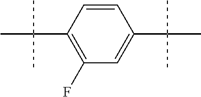 |
| 102. | 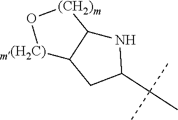 | 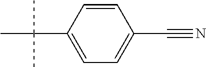 | 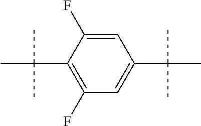 |
| 103. | 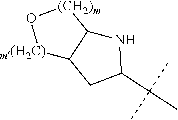 | 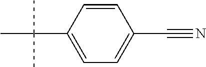 | 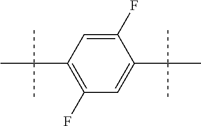 |
| 104. | 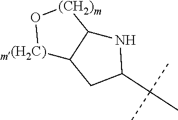 | 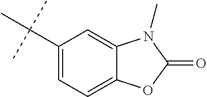 | 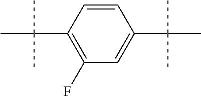 |
| 105. | 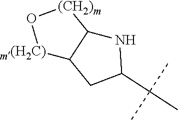 | 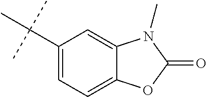 | 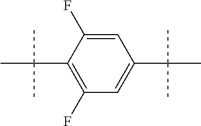 |
| 106. | 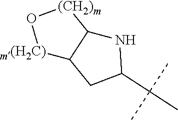 | 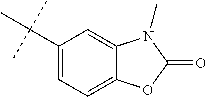 | 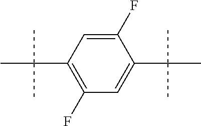 |
| 107. | 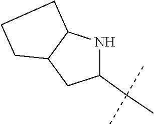 | 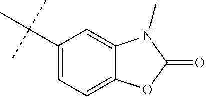 | 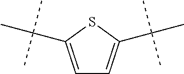 |
| 108. | 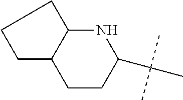 | 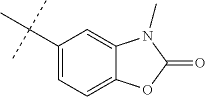 | 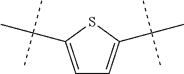 |
| 109. | 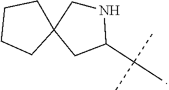 | 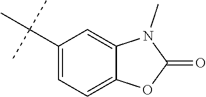 | 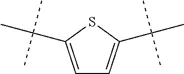 |
| 110. | 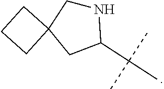 | 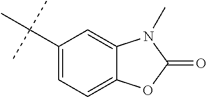 | 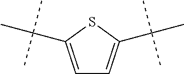 |
| 111. | 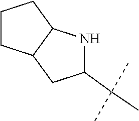 | 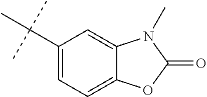 | 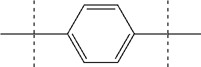 |
| 112. | 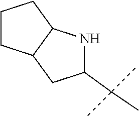 | 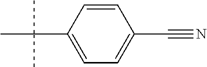 | 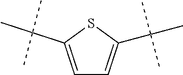 |
| 113. | 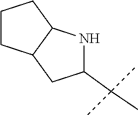 | 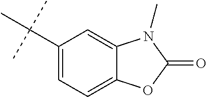 | 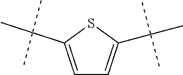 |
| 114. | 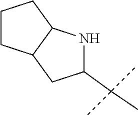 | 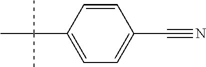 | 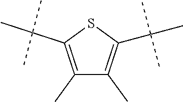 |
| 115. | 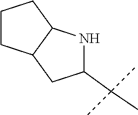 | 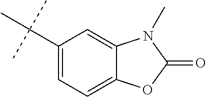 | 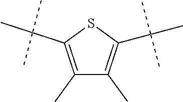 |
| 116. | 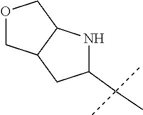 | 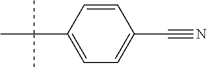 | 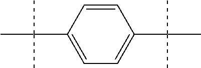 |
| 117. | 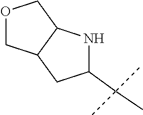 | 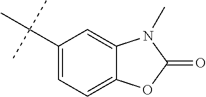 | 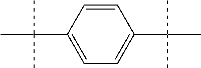 |
| 118. | 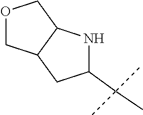 | 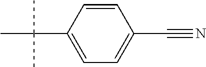 | 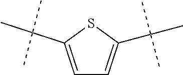 |
| 119. | 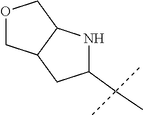 | 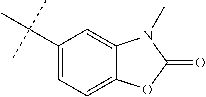 | 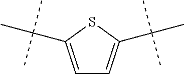 |
| 120. | 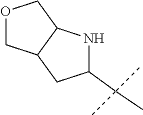 | 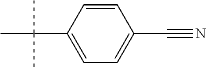 | 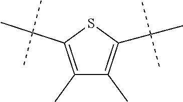 |
| 121. | 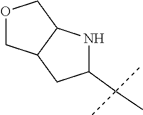 | 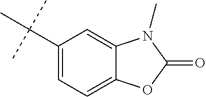 | 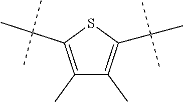 |

In another aspect of the invention, a DPP1 inhibitor of Formula (I), e.g., a compound of Formula (I-I), (IA), (IB), (IC), (ID), (IE), (I-I), (IA-I), (IB-I), (IC-I), (ID-I), or a pharmaceutically acceptable salt or deuterated form thereof, is one or more compounds selected from Table 4.

TABLE 4
Representative compounds of Formula (I).

| Com- | |
|---|---|
| pound | Structure |
| 122. | 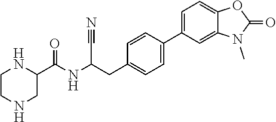 [SMILES: Cn1c(=O)oc2ccc(-c3ccc(CC(C#N)NC(=O)C4CNCCN4)cc3)cc21] |
| 123. | 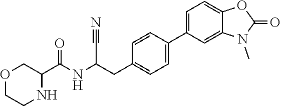 [SMILES: Cn1c(=O)oc2ccc(-c3ccc(CC(C#N)NC(=O)C4COCCN4)cc3)cc21] |
| 124. | 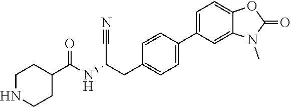 [SMILES: Cn1c(=O)oc2ccc(-c3ccc(C[C@@H](C#N)NC(=O)C4CCNCC4)cc3)cc21] |
| 125. | 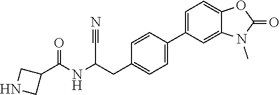 [SMILES: Cn1c(=O)oc2ccc(-c3ccc(CC(C#N)NC(=O)C4CNC4)cc3)cc21] |
| 126. | 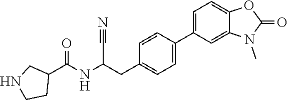 [SMILES: Cn1c(=O)oc2ccc(-c3ccc(CC(C#N)NC(=O)C4CCNC4)cc3)cc21] |
| 127. | 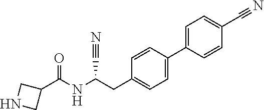 [SMILES: N#Cc1ccc(-c2ccc(C[C@@H](C#N)NC(=O)C3CNC3)cc2)cc1] |
| 128. | 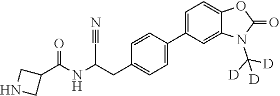 [SMILES: [2H]C([2H])([2H])n1c(=O)oc2ccc(-c3ccc(CC(C#N)NC(=O)C4CNC4)cc3)cc21] |
| 129. | 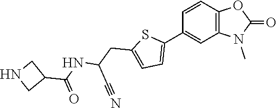 [SMILES: Cn1c(=O)oc2ccc(-c3ccc(CC(C#N)NC(=O)C4CNC4)s3)cc21] |
| 130. | 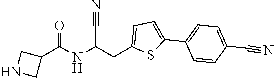 [SMILES: N#Cc1ccc(-c2ccc(CC(C#N)NC(=O)C3CNC3)s2)cc1] |
| 131. | 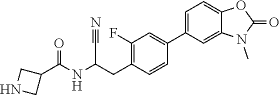 [SMILES: Cn1c(=O)oc2ccc(-c3ccc(CC(C#N)NC(=O)C4CNC4)c(F)c3)cc21] |
| 132. | 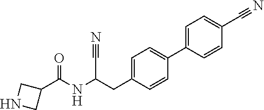 [SMILES: N#Cc1ccc(-c2ccc(CC(C#N)NC(=O)C3CNC3)cc2)cc1] |
| 133. | 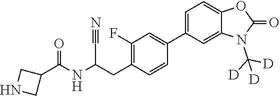 [SMILES: [2H]C([2H])([2H])n1c(=O)oc2ccc(-c3ccc(CC(C#N)NC(=O)C4CNC4)c(F)c3)cc21] |
| 134. | 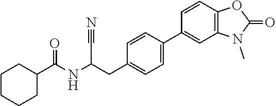 [SMILES: Cn1c(=O)oc2ccc(-c3ccc(CC(C#N)NC(=O)C4CCCCC4)cc3)cc21] |
| 135. | 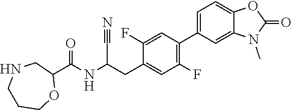 [SMILES: Cn1c(=O)oc2ccc(-c3cc(F)c(CC(C#N)NC(=O)C4CNCCCO4)cc3F)cc21] |
| 136. | 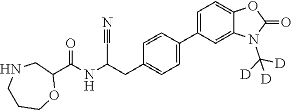 [SMILES: [2H]C([2H])([2H])n1c(=O)oc2ccc(-c3ccc(CC(C#N)NC(=O)C4CNCCCO4)cc3)cc21] |
| 137. | 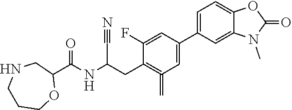 |
| 138. | 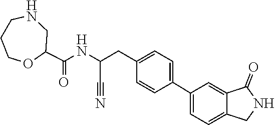 [SMILES: N#CC(Cc1ccc(-c2ccc3c(c2)C(=O)NC3)cc1)NC(=O)C1CNCCCO1] |

In another aspect of the invention, a DPP1 inhibitor of Formula (I), e.g., a compound of Formula (I-I), (IA), (IB), (IC), (ID), (I-E), (I-I), (IA-I), (IB-I), (IC-I), (ID-I), or a pharmaceutically acceptable salt or deuterated form thereof, is one or more compounds selected from Table 5.

TABLE 5
Representative compounds of Formula (I).

| Compound | Structure |
|---|---|
| 139. | 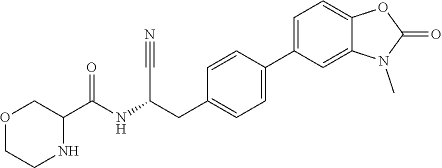 [SMILES: Cn1c(=O)oc2ccc(-c3ccc(C[C@@H](C#N)NC(=O)C4COCCN4)cc3)cc21] |
| 140. | 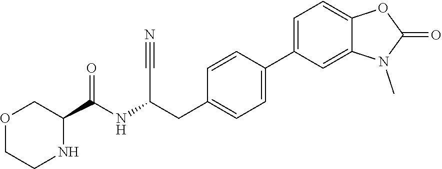 [SMILES: Cn1c(=O)oc2ccc(-c3ccc(C[C@@H](C#N)NC(=O)[C@@H]4COCCN4)cc3)cc21] |
| 141. | 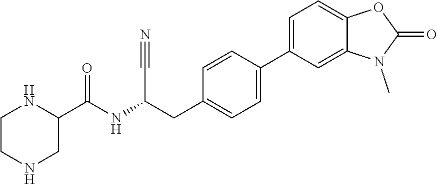 [SMILES: Cn1c(=O)oc2ccc(-c3ccc(C[C@@H](C#N)NC(=O)C4CNCCN4)cc3)cc21] |
| 142. | 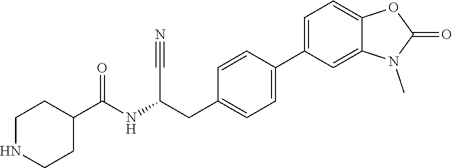 [SMILES: Cn1c(=O)oc2ccc(-c3ccc(C[C@@H](C#N)NC(=O)C4CCNCC4)cc3)cc21] |
| 143. | 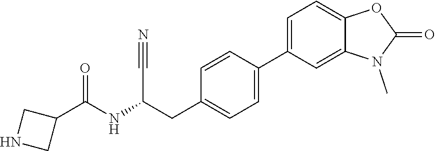 [SMILES: Cn1c(=O)oc2ccc(-c3ccc(C[C@@H](C#N)NC(=O)C4CNC4)cc3)cc21] |
| 144 | 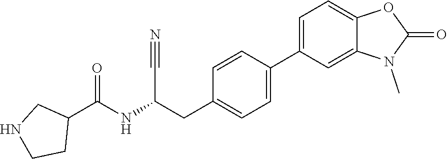 [SMILES: Cn1c(=O)oc2ccc(-c3ccc(C[C@@H](C#N)NC(=O)C4CCNC4)cc3)cc21] |
| 145. | 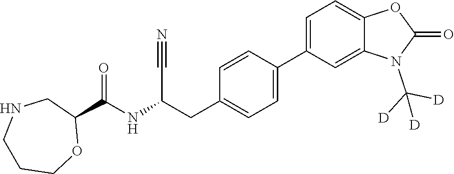 [SMILES: [2H]C([2H])([2H])n1c(=O)oc2ccc(-c3ccc(C[C@@H](C#N)NC(=O)[C@@H]4CNCCCO4)cc3)cc21] |
| 146. | 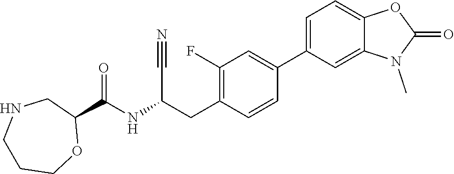 [SMILES: Cn1c(=O)oc2ccc(-c3ccc(C[C@@H](C#N)NC(=O)[C@@H]4CNCCCO4)c(F)c3)cc21] |
| 147. | 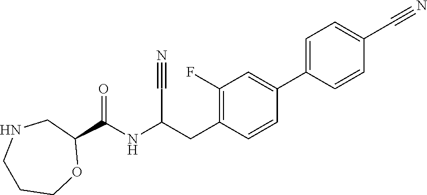 [SMILES: N#Cc1ccc(-c2ccc(CC(C#N)NC(=O)[C@@H]3CNCCCO3)c(F)c2)cc1] |
| 148. | 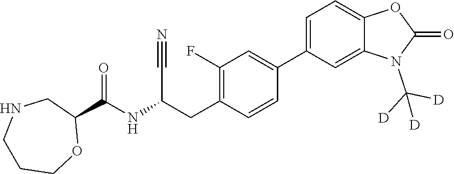 [SMILES: [2H]C([2H])([2H])n1c(=O)oc2ccc(-c3ccc(C[C@@H](C#N)NC(=O)[C@@H]4CNCCCO4)c(F)c3)cc21] |
| 149. | 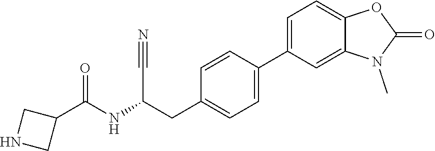 [SMILES: Cn1c(=O)oc2ccc(-c3ccc(C[C@@H](C#N)NC(=O)C4CNC4)cc3)cc21] |
| 150. | 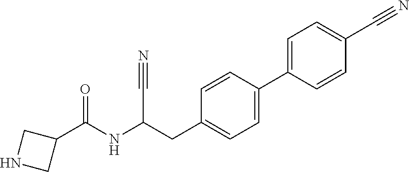 [SMILES: N#Cc1ccc(-c2ccc(CC(C#N)NC(=O)C3CNC3)cc2)cc1] |
| 151. | 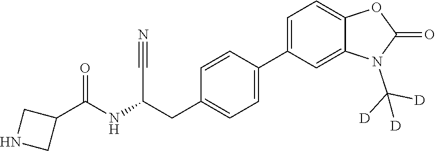 [SMILES: [2H]C([2H])([2H])n1c(=O)oc2ccc(-c3ccc(C[C@@H](C#N)NC(=O)C4CNC4)cc3)cc21] |
| 152. | 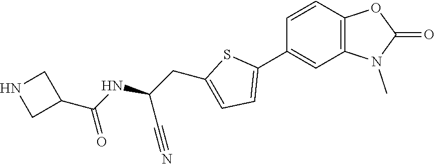 [SMILES: Cn1c(=O)oc2ccc(-c3ccc(C[C@@H](C#N)NC(=O)C4CNC4)s3)cc21] |
| 153. | 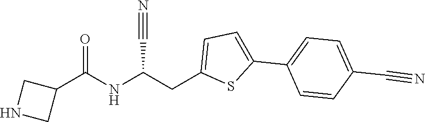 [SMILES: N#Cc1ccc(-c2ccc(C[C@@H](C#N)NC(=O)C3CNC3)s2)cc1] |
| 154. | 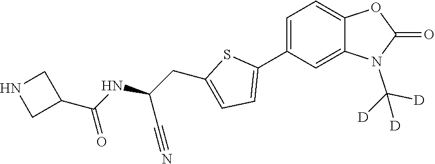 [SMILES: [2H]C([2H])([2H])n1c(=O)oc2ccc(-c3ccc(C[C@@H](C#N)NC(=O)C4CNC4)s3)cc21] |
| 155. | 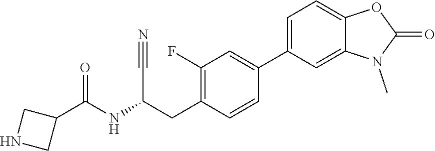 [SMILES: Cn1c(=O)oc2ccc(-c3ccc(C[C@@H](C#N)NC(=O)C4CNC4)c(F)c3)cc21] |
| 156. | 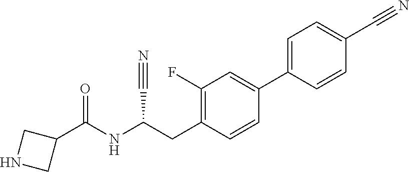 [SMILES: N#Cc1ccc(-c2ccc(C[C@@H](C#N)NC(=O)C3CNC3)c(F)c2)cc1] |
| 157. | 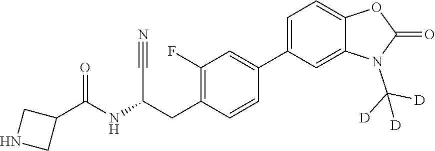 [SMILES: [2H]C([2H])([2H])n1c(=O)oc2ccc(-c3ccc(C[C@@H](C#N)NC(=O)C4CNC4)c(F)c3)cc21] |
| 158. | 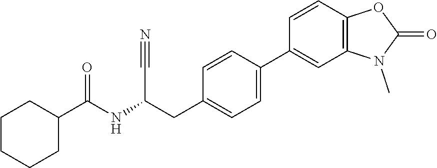 [SMILES: Cn1c(=O)oc2ccc(-c3ccc(C[C@@H](C#N)NC(=O)C4CCCCC4)cc3)cc21] |
| 159. | 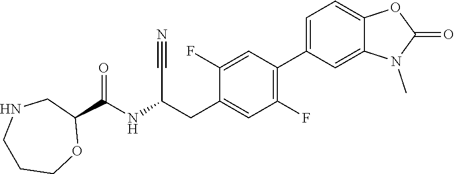 [SMILES: Cn1c(=O)oc2ccc(-c3cc(F)c(C[C@@H](C#N)NC(=O)[C@@H]4CNCCCO4)cc3F)cc21] |
| 160. | 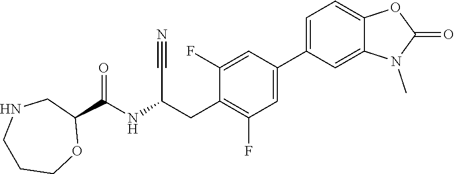 [SMILES: Cn1c(=O)oc2ccc(-c3cc(F)c(C[C@@H](C#N)NC(=O)[C@@H]4CNCCCO4)c(F)c3)cc21] |
| 161. | 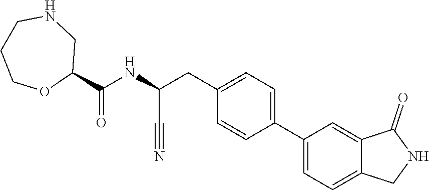 [SMILES: N#C[C@H](Cc1ccc(-c2ccc3c(c2)C(=O)NC3)cc1)NC(=O)[C@@H]1CNCCCO1] |
| 296. | 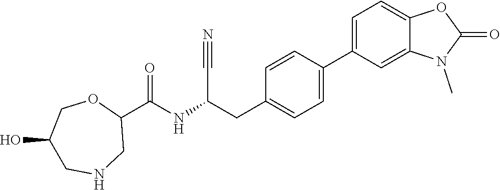 [SMILES: Cn1c(=O)oc2ccc(-c3ccc(C[C@@H](C#N)NC(=O)C4CNC[C@@H](O)CO4)cc3)cc21] |
| 297. | 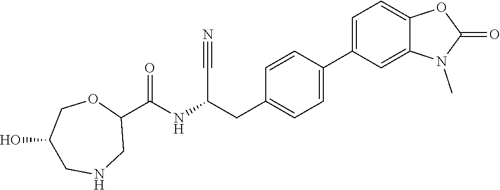 [SMILES: Cn1c(=O)oc2ccc(-c3ccc(C[C@@H](C#N)NC(=O)C4CNC[C@H](O)CO4)cc3)cc21] |
| 304. | 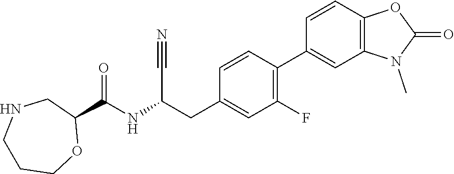 [SMILES: Cn1c(=O)oc2ccc(-c3ccc(C[C@@H](C#N)NC(=O)[C@@H]4CNCCCO4)cc3F)cc21] |
| 305. | 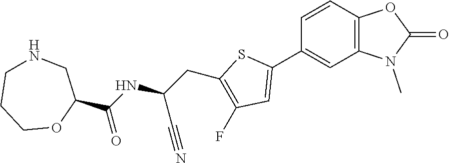 [SMILES: Cn1c(=O)oc2ccc(-c3cc(F)c(C[C@@H](C#N)NC(=O)[C@@H]4CNCCCO4)s3)cc21] |
| 310. | 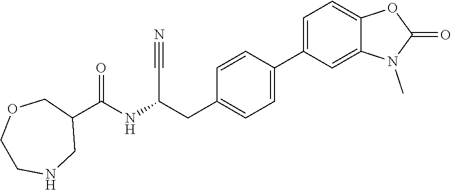 [SMILES: Cn1c(=O)oc2ccc(-c3ccc(C[C@@H](C#N)NC(=O)C4CNCCOC4)cc3)cc21] |

In another aspect of the invention, a DPP1 inhibitor of Formula (I), e.g., a compound of Formula (I-I), (IA), (IB), (IC), (ID), (IE), (I-I), (IA-I), (IB-I), (IC-I), (ID-I), or a pharmaceutically acceptable salt or deuterated form thereof, is one or more compounds selected from Table 6.

TABLE 6
Representative compounds of Formula (I).

| compound | Structure |
|---|---|
| 162. | 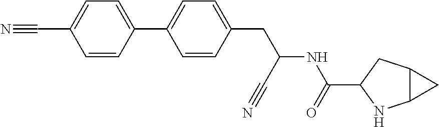 [SMILES: N#Cc1ccc(-c2ccc(CC(C#N)NC(=O)C3CC4CC4N3)cc2)cc1] |
| 163. | 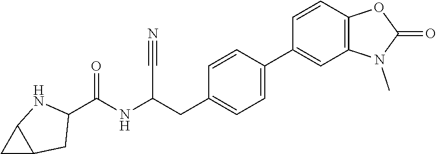 [SMILES: Cn1c(=O)oc2ccc(-c3ccc(CC(C#N)NC(=O)C4CC5CC5N4)cc3)cc21] |
| 164. | 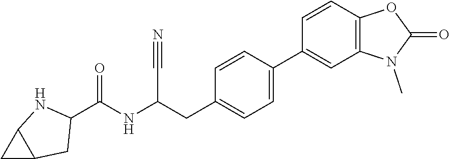 [SMILES: Cn1c(=O)oc2ccc(-c3ccc(CC(C#N)NC(=O)C4CC5CC5N4)cc3)cc21] |
| 165. | 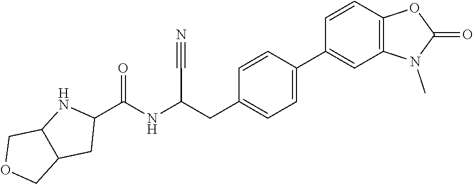 [SMILES: Cn1c(=O)oc2ccc(-c3ccc(CC(C#N)NC(=O)C4CC5COCC5N4)cc3)cc21] |
| 166. | 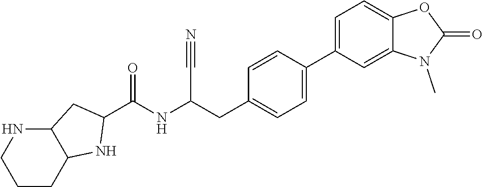 [SMILES: Cn1c(=O)oc2ccc(-c3ccc(CC(C#N)NC(=O)C4CC5NCCCC5N4)cc3)cc21] |
| 167. | 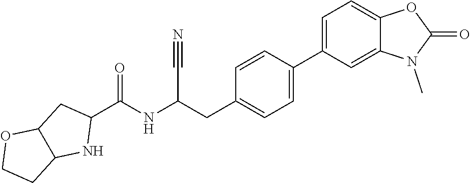 [SMILES: Cn1c(=O)oc2ccc(-c3ccc(CC(C#N)NC(=O)C4CC5OCCC5N4)cc3)cc21] |
| 168. | 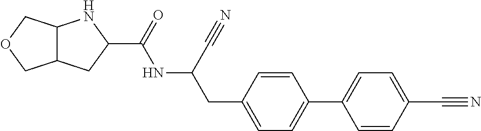 [SMILES: N#Cc1ccc(-c2ccc(CC(C#N)NC(=O)C3CC4COCC4N3)cc2)cc1] |
| 169. | 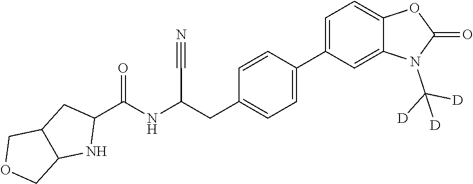 [SMILES: [2H]C([2H])([2H])n1c(=O)oc2ccc(-c3ccc(CC(C#N)NC(=O)C4CC5COCC5N4)cc3)cc21] |
| 170. | 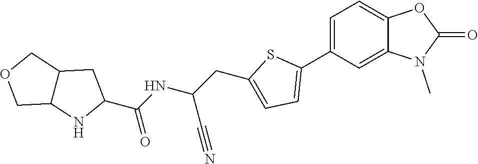 [SMILES: Cn1c(=O)oc2ccc(-c3ccc(CC(C#N)NC(=O)C4CC5COCC5N4)s3)cc21] |
| 171. | 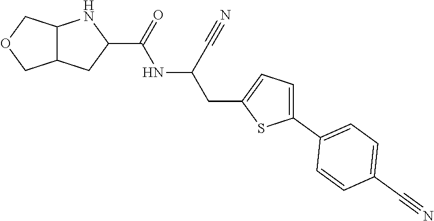 [SMILES: N#Cc1ccc(-c2ccc(CC(C#N)NC(=O)C3CC4COCC4N3)s2)cc1] |
| 172. | 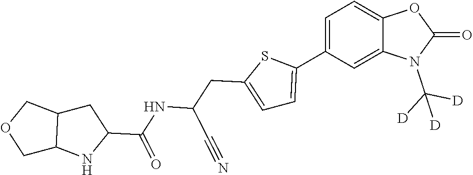 [SMILES: [2H]C([2H])([2H])n1c(=O)oc2ccc(-c3ccc(CC(C#N)NC(=O)C4CC5COCC5N4)s3)cc21] |
| 173. | 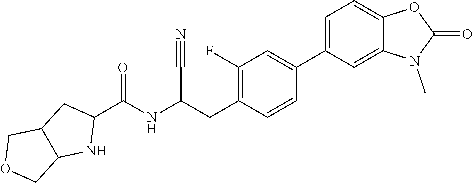 [SMILES: Cn1c(=O)oc2ccc(-c3ccc(CC(C#N)NC(=O)C4CC5COCC5N4)c(F)c3)cc21] |
| 174. | 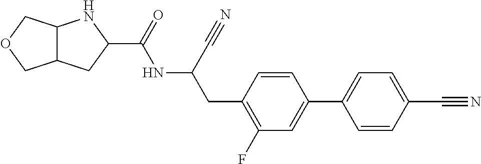 [SMILES: N#Cc1ccc(-c2ccc(CC(C#N)NC(=O)C3CC4COCC4N3)c(F)c2)cc1] |
| 175. | 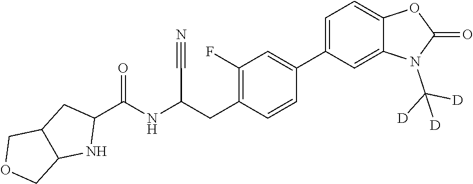 [SMILES: [2H]C([2H])([2H])n1c(=O)oc2ccc(-c3ccc(CC(C#N)NC(=O)C4CC5COCC5N4)c(F)c3)cc21] |
| 176. | 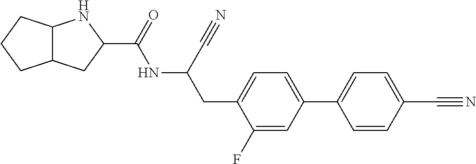 [SMILES: N#Cc1ccc(-c2ccc(CC(C#N)NC(=O)C3CC4CCCC4N3)c(F)c2)cc1] |
| 177. | 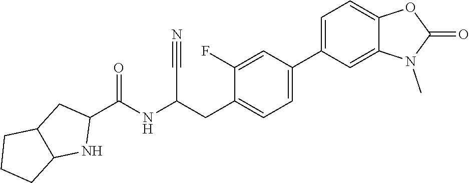 [SMILES: Cn1c(=O)oc2ccc(-c3ccc(CC(C#N)NC(=O)C4CC5CCCC5N4)c(F)c3)cc21] |
| 178. | 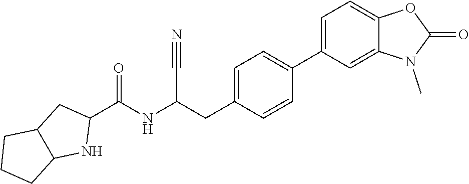 [SMILES: Cn1c(=O)oc2ccc(-c3ccc(CC(C#N)NC(=O)C4CC5CCCC5N4)cc3)cc21] |

In another aspect of the invention, a DPP1 inhibitor of Formula (I), e.g., a compound of Formula (I-I), (IA), (IB), (IC), (ID), (IE), (I-I), (IA-I), (IB-I), (IC-I), (ID-I), or a pharmaceutically acceptable salt or deuterated form thereof, is one or more compounds selected from Table 7.

TABLE 7
Representative compounds of Formula (I).

| Compound | Structure |
|---|---|
| 179. | 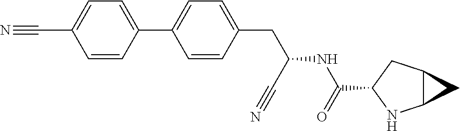 [SMILES: N#Cc1ccc(-c2ccc(C[C@@H](C#N)NC(=O)[C@@H]3C[C@H]4C[C@H]4N3)cc2)cc1] |
| 180. | 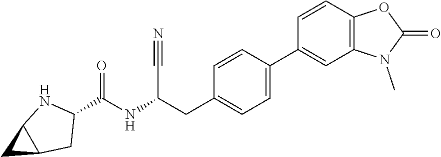 [SMILES: Cn1c(=O)oc2ccc(-c3ccc(C[C@@H](C#N)NC(=O)[C@@H]4C[C@H]5C[C@H]5N4)cc3)cc21] |
| 181. | 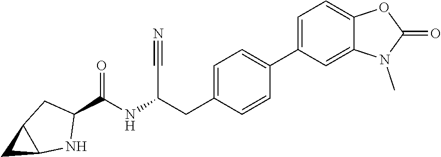 [SMILES: Cn1c(=O)oc2ccc(-c3ccc(C[C@@H](C#N)NC(=O)[C@@H]4C[C@@H]5C[C@@H]5N4)cc3)cc21] |
| 182. | 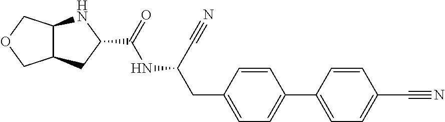 [SMILES: N#Cc1ccc(-c2ccc(C[C@@H](C#N)NC(=O)[C@@H]3C[C@H]4COC[C@H]4N3)cc2)cc1] |
| 183. | 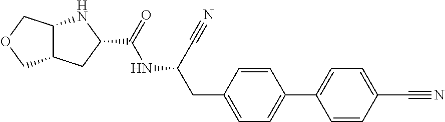 [SMILES: N#Cc1ccc(-c2ccc(C[C@@H](C#N)NC(=O)[C@@H]3C[C@@H]4COC[C@@H]4N3)cc2)cc1] |
| 184. | 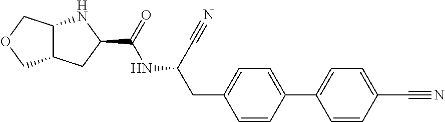 [SMILES: N#Cc1ccc(-c2ccc(C[C@@H](C#N)NC(=O)[C@H]3C[C@@H]4COC[C@@H]4N3)cc2)cc1] |
| 185. | 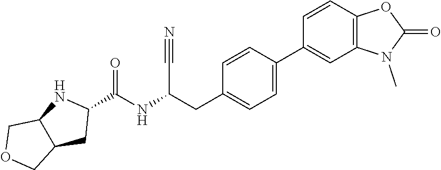 [SMILES: Cn1c(=O)oc2ccc(-c3ccc(C[C@@H](C#N)NC(=O)[C@@H]4C[C@H]5COC[C@H]5N4)cc3)cc21] |
| 186. | 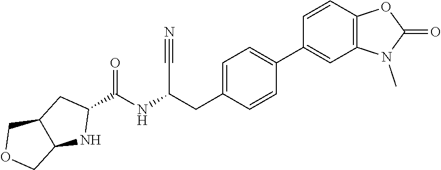 [SMILES: Cn1c(=O)oc2ccc(-c3ccc(C[C@@H](C#N)NC(=O)[C@H]4C[C@@H]5COC[C@@H]5N4)cc3)cc21] |
| 187. | 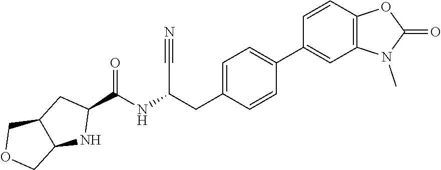 [SMILES: Cn1c(=O)oc2ccc(-c3ccc(C[C@@H](C#N)NC(=O)[C@@H]4C[C@@H]5COC[C@@H]5N4)cc3)cc21] |
| 188. | 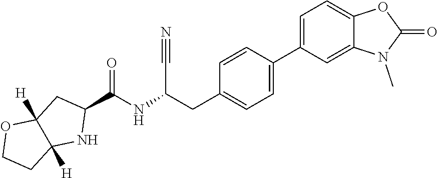 [SMILES: Cn1c(=O)oc2ccc(-c3ccc(C[C@@H](C#N)NC(=O)[C@@H]4C[C@H]5OCC[C@H]5N4)cc3)cc21] |
| 189. | 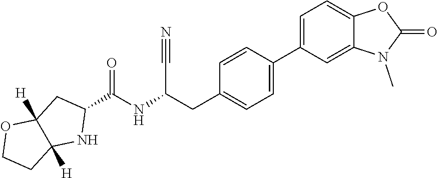 [SMILES: Cn1c(=O)oc2ccc(-c3ccc(C[C@@H](C#N)NC(=O)[C@H]4C[C@H]5OCC[C@H]5N4)cc3)cc21] |
| 190. | 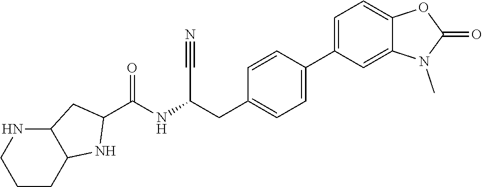 [SMILES: Cn1c(=O)oc2ccc(-c3ccc(C[C@@H](C#N)NC(=O)C4CC5NCCCC5N4)cc3)cc21] |
| 191. | 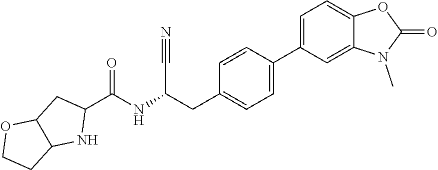 [SMILES: Cn1c(=O)oc2ccc(-c3ccc(C[C@@H](C#N)NC(=O)C4CC5OCCC5N4)cc3)cc21] |
| 192. | 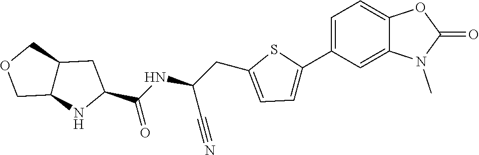 [SMILES: Cn1c(=O)oc2ccc(-c3ccc(C[C@@H](C#N)NC(=O)[C@@H]4C[C@@H]5COC[C@@H]5N4)s3)cc21] |
| 193. | 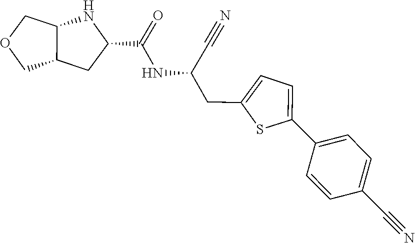 [SMILES: N#Cc1ccc(-c2ccc(C[C@@H](C#N)NC(=O)[C@@H]3C[C@@H]4COC[C@@H]4N3)s2)cc1] |
| 194. | 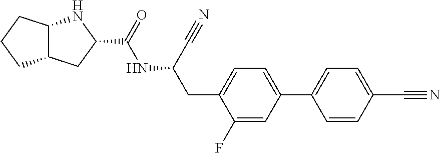 [SMILES: N#Cc1ccc(-c2ccc(C[C@@H](C#N)NC(=O)[C@@H]3C[C@@H]4CCC[C@@H]4N3)c(F)c2)cc1] |
| 195. | 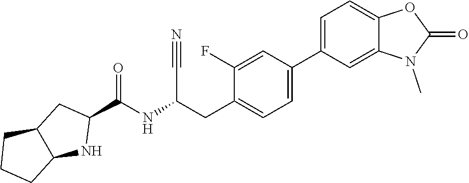 [SMILES: Cn1c(=O)oc2ccc(-c3ccc(C[C@@H](C#N)NC(=O)[C@@H]4C[C@@H]5CCC[C@@H]5N4)c(F)c3)cc21] |
| 196. | 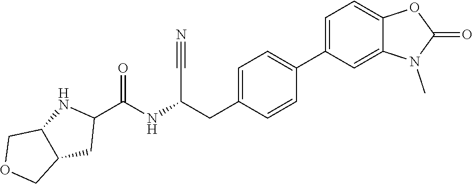 |
| 197. | 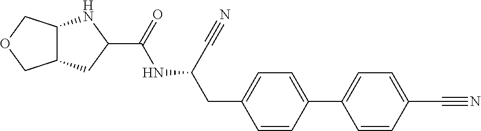 |
| 293 | 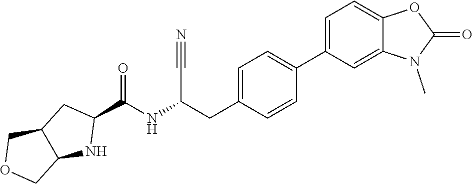 [SMILES: Cn1c(=O)oc2ccc(-c3ccc(C[C@@H](C#N)NC(=O)[C@@H]4C[C@@H]5COC[C@@H]5N4)cc3)cc21] |
| 298 | 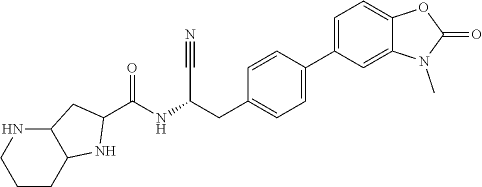 [SMILES: Cn1c(=O)oc2ccc(-c3ccc(C[C@@H](C#N)NC(=O)C4CC5NCCCC5N4)cc3)cc21] |
| 299 | 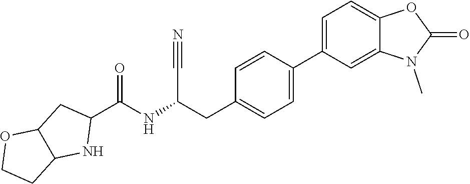 [SMILES: Cn1c(=O)oc2ccc(-c3ccc(C[C@@H](C#N)NC(=O)C4CC5OCCC5N4)cc3)cc21] |
| 300 | 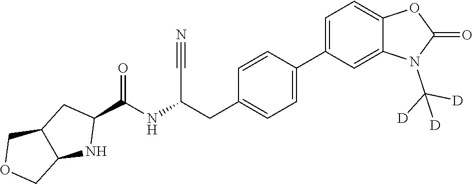 [SMILES: [2H]C([2H])([2H])n1c(=O)oc2ccc(-c3ccc(C[C@@H](C#N)NC(=O)[C@@H]4C[C@@H]5COC[C@@H]5N4)cc3)cc21] |
| 301 | 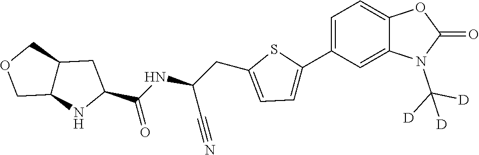 [SMILES: [2H]C([2H])([2H])n1c(=O)oc2ccc(-c3ccc(C[C@@H](C#N)NC(=O)[C@@H]4C[C@@H]5COC[C@@H]5N4)s3)cc21] |
| 302 | 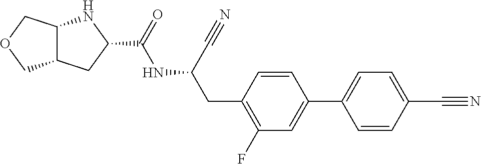 [SMILES: N#Cc1ccc(-c2ccc(C[C@@H](C#N)NC(=O)[C@@H]3C[C@@H]4COC[C@@H]4N3)c(F)c2)cc1] |
| 303 | 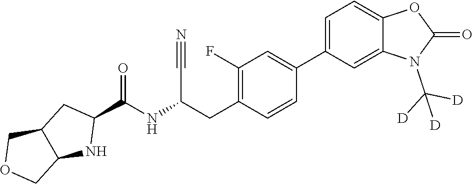 [SMILES: [2H]C([2H])([2H])n1c(=O)oc2ccc(-c3ccc(C[C@@H](C#N)NC(=O)[C@@H]4C[C@@H]5COC[C@@H]5N4)c(F)c3)cc21] |
| 306 | 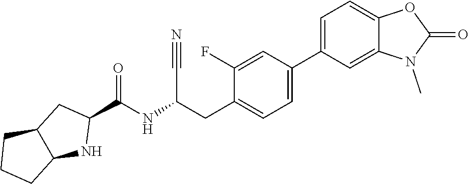 [SMILES: Cn1c(=O)oc2ccc(-c3ccc(C[C@@H](C#N)NC(=O)[C@@H]4C[C@@H]5CCC[C@@H]5N4)c(F)c3)cc21] |
| 307 | 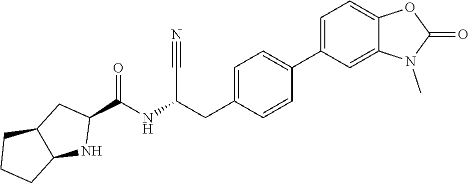 [SMILES: Cn1c(=O)oc2ccc(-c3ccc(C[C@@H](C#N)NC(=O)[C@@H]4C[C@@H]5CCC[C@@H]5N4)cc3)cc21] |
| 309 | 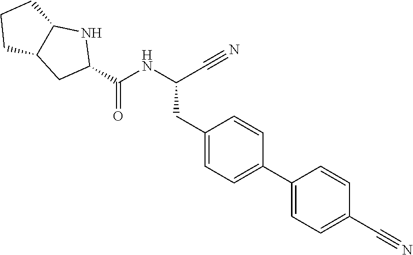 [SMILES: N#Cc1ccc(-c2ccc(C[C@@H](C#N)NC(=O)[C@@H]3C[C@@H]4CCC[C@@H]4N3)cc2)cc1] |
| 259 | 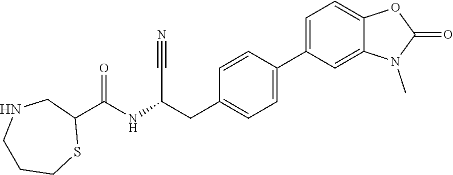 [SMILES: Cn1c(=O)oc2ccc(-c3ccc(C[C@@H](C#N)NC(=O)C4CNCCCS4)cc3)cc21] |
| 259a | 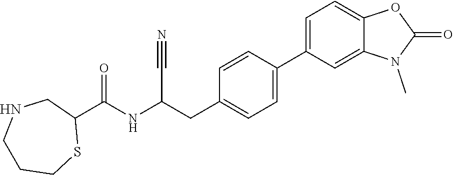 [SMILES: Cn1c(=O)oc2ccc(-c3ccc(CC(C#N)NC(=O)C4CNCCCS4)cc3)cc21] |

### Formula (II)

In another aspect of the invention, a DPP1 inhibitor of Formula (II), or a pharmaceutically acceptable salt or deuterated form thereof, is provided:

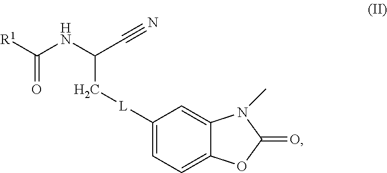

wherein,
R¹ is

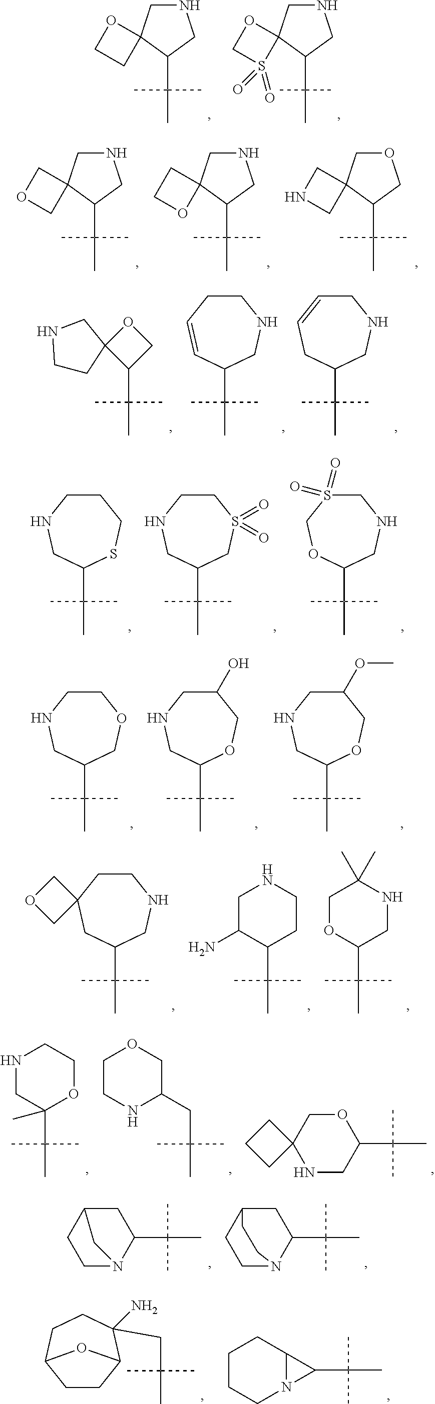

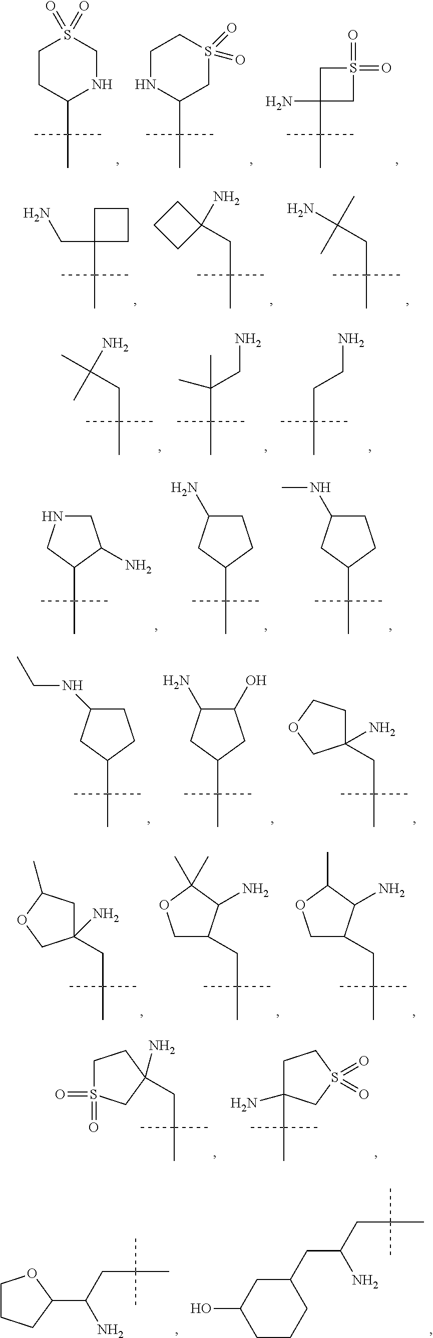

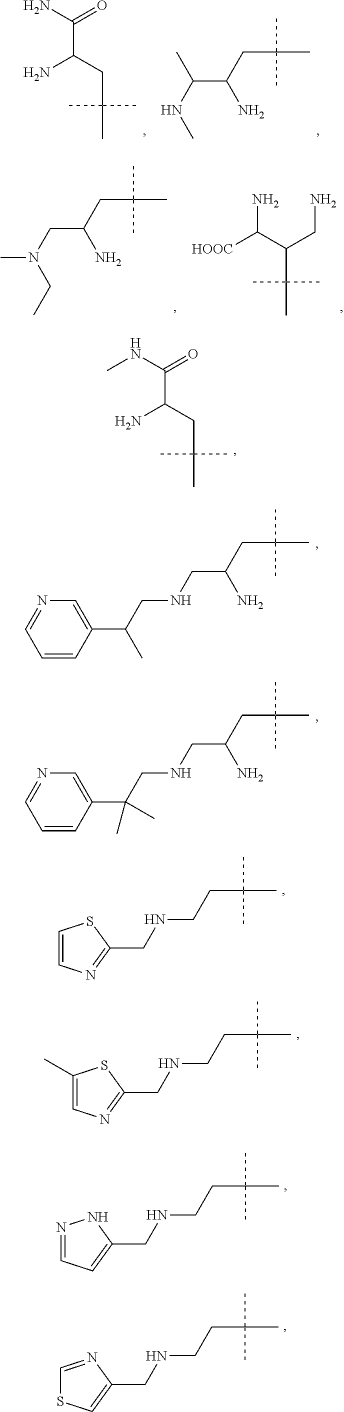

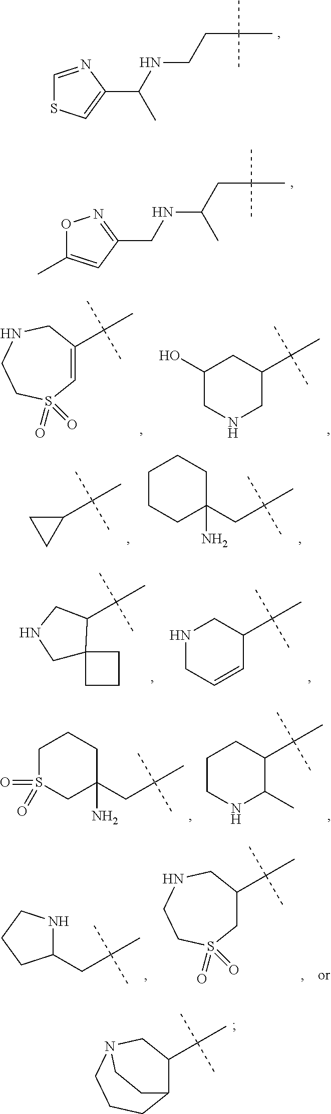

L is

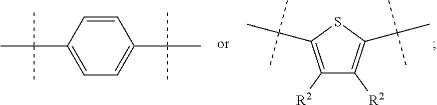

and
each R² is independently H, F, Cl, Br, I or C₁-C₆ alkyl, wherein the compound is not

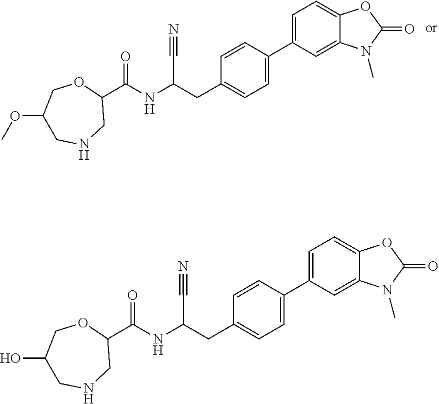 [SMILES: COC1CNCC(C(=O)NC(C#N)Cc2ccc(-c3ccc4oc(=O)n(C)c4c3)cc2)OC1 ; "or" ; Cn1c(=O)oc2ccc(-c3ccc(CC(C#N)NC(=O)C4CNCC(O)CO4)cc3)cc21]

In one embodiment of a compound of Formula (II), or a pharmaceutically acceptable salt or deuterated form thereof, R¹ is

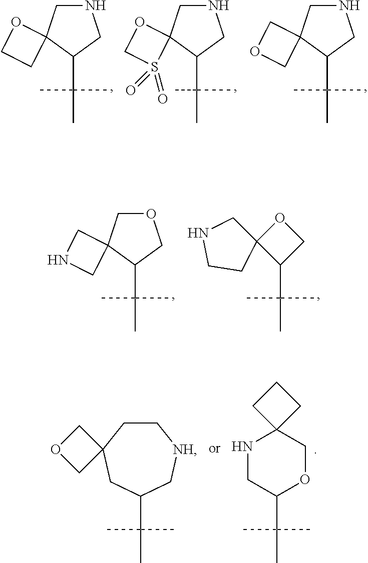

In a further embodiment, R² is

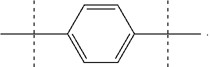

In another embodiment of a compound of Formula (II), R¹ is

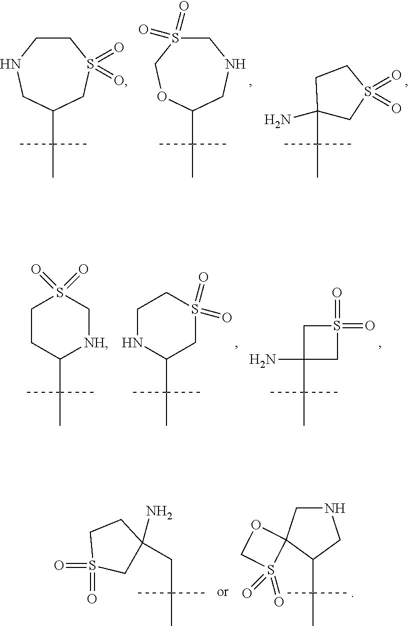

In a further embodiment, R² is

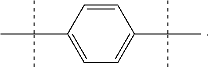

In yet another embodiment of a compound of Formula (II), R¹ is

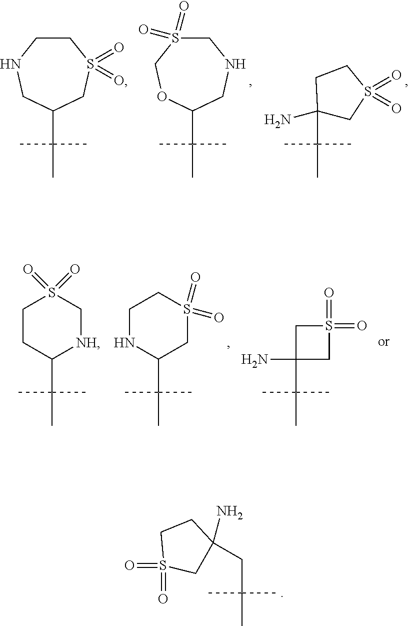

In a further embodiment, R² is

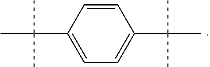

In even another embodiment of a compound of Formula (II), or a pharmaceutically acceptable salt or deuterated form thereof, R¹ is

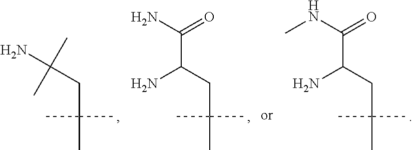

In a further embodiment, R² is

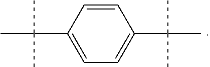

In yet even another embodiment of a compound of Formula (II), or a pharmaceutically acceptable salt or deuterated form thereof, R¹ is

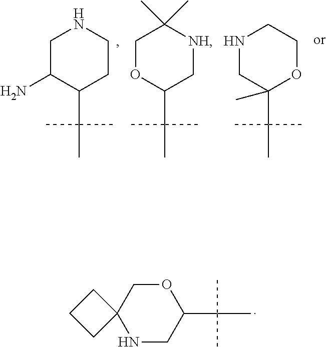

In a further embodiment, R² is

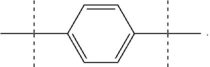

In one embodiment of a compound of Formula (II), or a pharmaceutically acceptable salt or deuterated form thereof, R¹

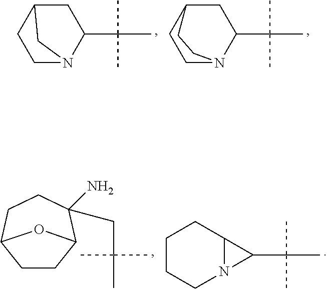

In a further embodiment, R² is

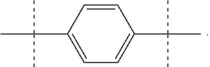

In even another embodiment of a compound of Formula (II), R¹ is

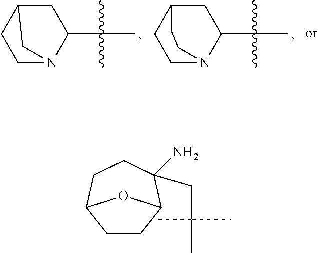

In a further embodiment, R² is

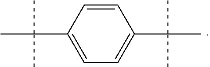

In even another embodiment of a compound of Formula (II), R¹ is

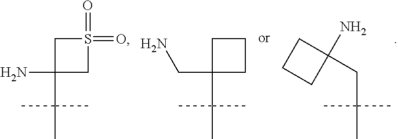

In a further embodiment, R² is

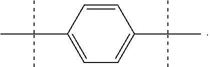

In another embodiment of a compound of Formula (II), R¹ is

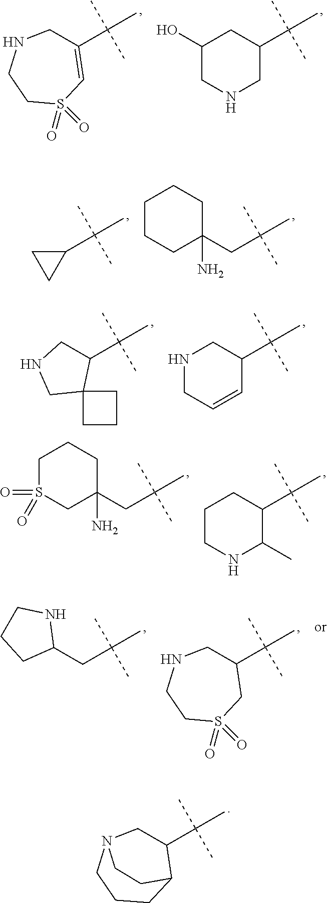

In a further embodiment, R² is

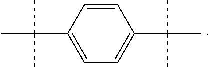

In one embodiment of a compound of Formula (II), or a pharmaceutically acceptable salt or deuterated form thereof, R¹ is

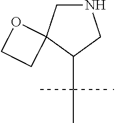

In a further embodiment, R² is

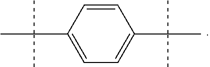

In one embodiment of a compound of Formula (II), or a pharmaceutically acceptable salt or deuterated form thereof, R¹ is

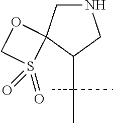

In a further embodiment, R² is

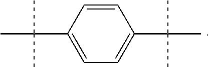

In one embodiment of a compound of Formula (II), or a pharmaceutically acceptable salt or deuterated form thereof, R¹ is

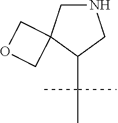

In a further embodiment, R² is

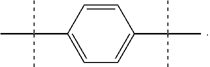

In one embodiment of a compound of Formula (II), or a pharmaceutically acceptable salt or deuterated form thereof, R¹ is

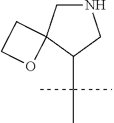

In a further embodiment, R² is

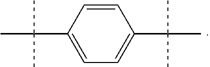

In one embodiment of a compound of Formula (II), or a pharmaceutically acceptable salt or deuterated form thereof, R¹ is

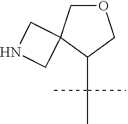

In a further embodiment, R² is

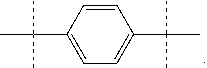

In one embodiment of a compound of Formula (II), or a pharmaceutically acceptable salt or deuterated form thereof, R¹ is

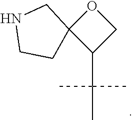

In a further embodiment, R² is

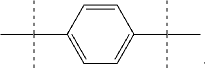

In one embodiment of a compound of Formula (II), or a pharmaceutically acceptable salt or deuterated form thereof, R¹ is

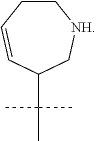

In a further embodiment, R² is

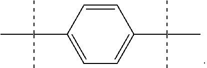

In one embodiment of a compound of Formula (II), or a pharmaceutically acceptable salt or deuterated form thereof, R¹ is

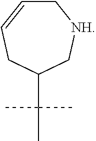

In a further embodiment, R² is

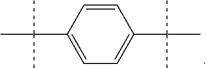

In one embodiment of a compound of Formula (II), or a pharmaceutically acceptable salt or deuterated form thereof, R¹ is

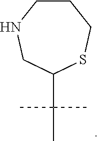

In a further embodiment, R² is

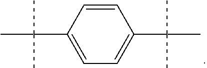

In one embodiment of a compound of Formula (II), or a pharmaceutically acceptable salt or deuterated form thereof, R¹ is

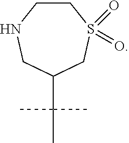

In a further embodiment, R² is

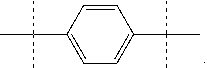

In one embodiment of a compound of Formula (II), or a pharmaceutically acceptable salt or deuterated form thereof, R¹ is

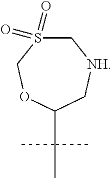

In a further embodiment, R² is

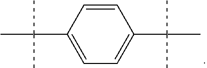

In one embodiment of a compound of Formula (II), or a pharmaceutically acceptable salt or deuterated form thereof, R¹ is

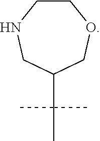

In a further embodiment, R² is

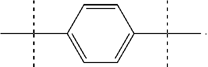

In one embodiment of a compound of Formula (II), or a pharmaceutically acceptable salt or deuterated form thereof, R¹ is

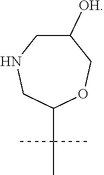

In a further embodiment, R² is

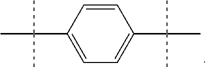

In one embodiment of a compound of Formula (II), or a pharmaceutically acceptable salt or deuterated form thereof, R¹ is

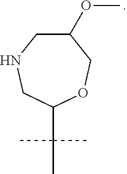

In a further embodiment, R² is

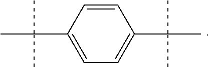

In one embodiment of a compound of Formula (II), or a pharmaceutically acceptable salt or deuterated form thereof, R¹ is

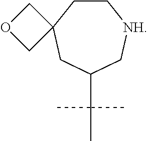

In a further embodiment, R² is

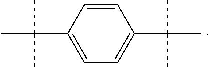

In one embodiment of a compound of Formula (II), or a pharmaceutically acceptable salt or deuterated form thereof, R¹ is

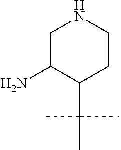

In a further embodiment, R² is

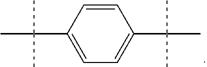

In one embodiment of a compound of Formula (II), or a pharmaceutically acceptable salt or deuterated form thereof, R¹ is

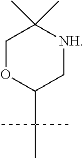

In a further embodiment, R² is

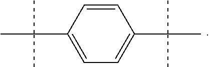

In one embodiment of a compound of Formula (II), or a pharmaceutically acceptable salt or deuterated form thereof, R¹ is

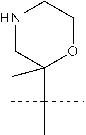

In a further embodiment, R² is

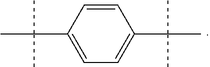

In one embodiment of a compound of Formula (II), or a pharmaceutically acceptable salt or deuterated form thereof, R¹ is

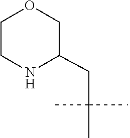

In a further embodiment, R² is

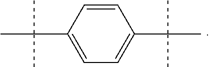

In one embodiment of a compound of Formula (II), or a pharmaceutically acceptable salt or deuterated form thereof, R¹ is

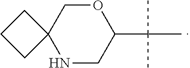

In a further embodiment, R² is

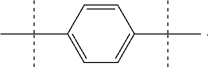

In one embodiment of a compound of Formula (II), or a pharmaceutically acceptable salt or deuterated form thereof, R¹ is

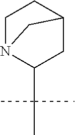

In a further embodiment, R² is

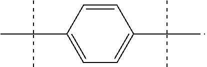

In one embodiment of a compound of Formula (II), or a pharmaceutically acceptable salt or deuterated form thereof, R¹ is

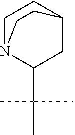

In a further embodiment, R² is

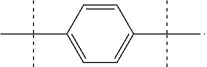

In one embodiment of a compound of Formula (II), or a pharmaceutically acceptable salt or deuterated form thereof, R¹ is

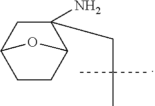

In a further embodiment, R² is

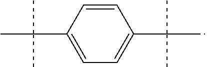

In one embodiment of a compound of Formula (II), or a pharmaceutically acceptable salt or deuterated form thereof, R¹ is

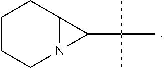

In a further embodiment, R² is

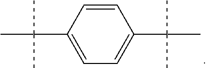

In one embodiment of a compound of Formula (II), or a pharmaceutically acceptable salt or deuterated form thereof, R¹ is

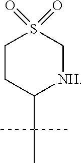

In a further embodiment, R² is

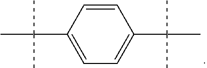

In one embodiment of a compound of Formula (II), or a pharmaceutically acceptable salt or deuterated form thereof, R¹ is

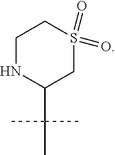

In a further embodiment, R² is

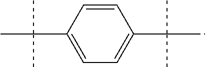

In one embodiment of a compound of Formula (II), or a pharmaceutically acceptable salt or deuterated form thereof, R¹ is

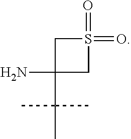

In a further embodiment, R² is

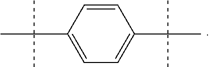

In one embodiment of a compound of Formula (II), or a pharmaceutically acceptable salt or deuterated form thereof, R¹ is

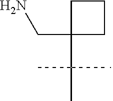

In a further embodiment, R² is

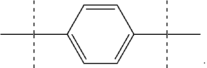

In one embodiment of a compound of Formula (II), or a pharmaceutically acceptable salt or deuterated form thereof, R¹ is

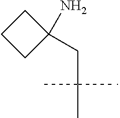

In a further embodiment, R² is

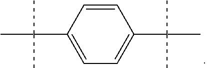

In one embodiment of a compound of Formula (II), or a pharmaceutically acceptable salt or deuterated form thereof, R¹ is

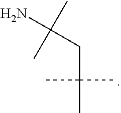

In a further embodiment, R² is

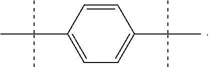

In one embodiment of a compound of Formula (II), or a pharmaceutically acceptable salt or deuterated form thereof, R¹ is

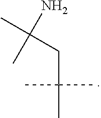

In a further embodiment, R² is

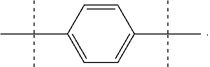

In one embodiment of a compound of Formula (II), or a pharmaceutically acceptable salt or deuterated form thereof, R¹ is

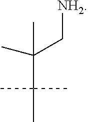

In a further embodiment, R² is

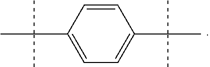

In one embodiment of a compound of Formula (II), or a pharmaceutically acceptable salt or deuterated form thereof, R¹ is

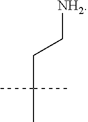

In a further embodiment, R² is

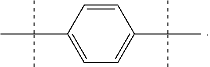

In one embodiment of a compound of Formula (II), or a pharmaceutically acceptable salt or deuterated form thereof, R¹ is

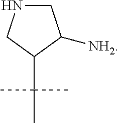

In a further embodiment, R² is

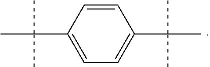

In one embodiment of a compound of Formula (II), or a pharmaceutically acceptable salt or deuterated form thereof, R¹ is

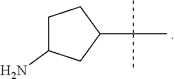

In a further embodiment, R² is

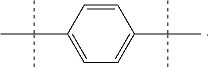

In one embodiment of a compound of Formula (II), or a pharmaceutically acceptable salt thereof, R¹ is

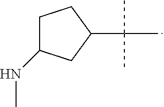

In a further embodiment, R² is

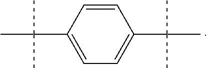

In one embodiment of a compound of Formula (II), or a pharmaceutically acceptable salt or deuterated form thereof, R¹ is

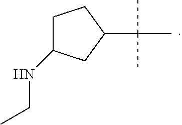

In a further embodiment, R² is

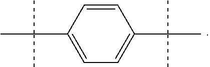

In one embodiment of a compound of Formula (II), or a pharmaceutically acceptable salt or deuterated form thereof, R¹ is

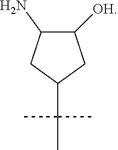

In a further embodiment, R² is

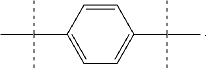

In one embodiment of a compound of Formula (II), or a pharmaceutically acceptable salt or deuterated form thereof, R¹ is

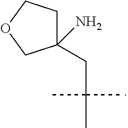

In a further embodiment, R² is

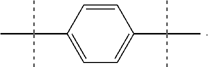

In one embodiment of a compound of Formula (II), or a pharmaceutically acceptable salt or deuterated form thereof, R¹ is

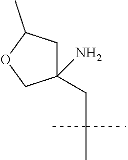

In a further embodiment, R² is

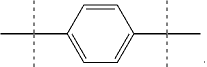

In one embodiment of a compound of Formula (II), or a pharmaceutically acceptable salt or deuterated form thereof, R¹ is

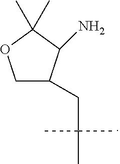

In a further embodiment, R² is

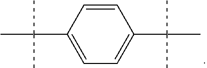

In one embodiment of a compound of Formula (II), or a pharmaceutically acceptable salt or deuterated form thereof, R¹ is

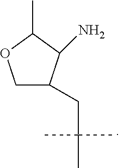

In a further embodiment, R² is

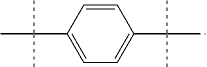

In one embodiment of a compound of Formula (II), or a pharmaceutically acceptable salt or deuterated form thereof, R¹ is

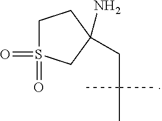

In a further embodiment, R² is

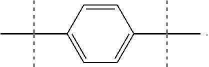

In one embodiment of a compound of Formula (II), or a pharmaceutically acceptable salt or deuterated form thereof, R¹ is

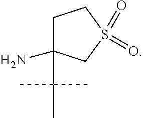

In a further embodiment, R² is

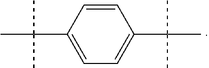

In one embodiment of a compound of Formula (II), or a pharmaceutically acceptable salt or deuterated form thereof, R¹ is

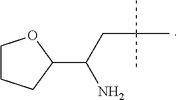

In a further embodiment, R² is

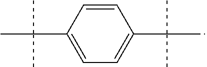

In one embodiment of a compound of Formula (II), or a pharmaceutically acceptable salt or deuterated form thereof, R¹ is

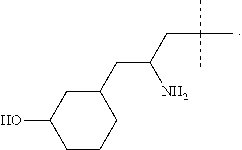

In a further embodiment, R² is

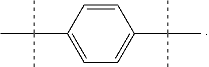

In one embodiment of a compound of Formula (II), or a pharmaceutically acceptable salt thereof, R¹ is

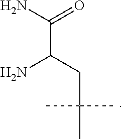

In a further embodiment, R² is

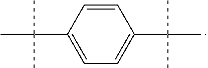

In one embodiment of a compound of Formula (II), or a pharmaceutically acceptable salt or deuterated form thereof, R¹ is

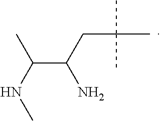

In a further embodiment, R² is

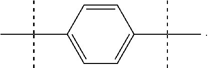

In one embodiment of a compound of Formula (II), or a pharmaceutically acceptable salt or deuterated form thereof, R¹ is

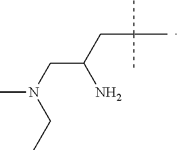

In a further embodiment, R² is

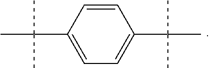

In one embodiment of a compound of Formula (II), or a pharmaceutically acceptable salt or deuterated form thereof, R¹ is

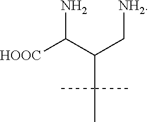

In a further embodiment, R² is

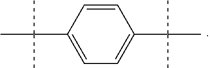

In one embodiment of a compound of Formula (II), or a pharmaceutically acceptable salt or deuterated form thereof, R¹ is

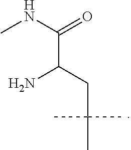

In a further embodiment, R² is

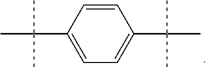

In one embodiment of a compound of Formula (II), or a pharmaceutically acceptable salt or deuterated form thereof, R¹ is

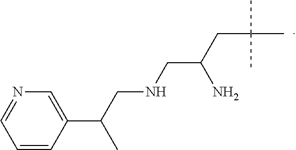

In a further embodiment, R² is

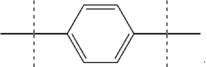

In one embodiment of a compound of Formula (II), or a pharmaceutically acceptable salt or deuterated form thereof, R¹ is

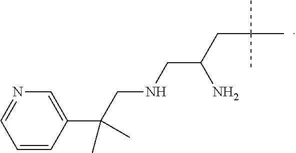

In a further embodiment, R² is

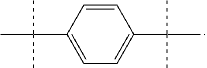

In one embodiment of a compound of Formula (II), or a pharmaceutically acceptable

salt or deuterated form thereof, R¹ is

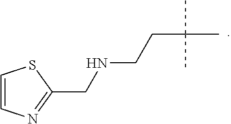

In a further embodiment, R² is

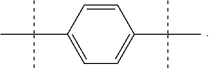

In one embodiment of a compound of Formula (II), or a pharmaceutically acceptable

salt or deuterated form thereof, R¹ is

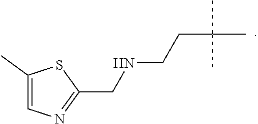

In a further embodiment, R² is

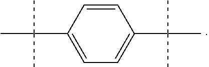

In one embodiment of a compound of Formula (II), or a pharmaceutically acceptable salt or deuterated form thereof, R¹ is

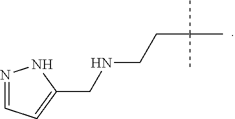

In a further embodiment, R² is

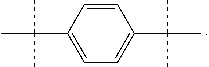

In one embodiment of a compound of Formula (II), or a pharmaceutically acceptable salt thereof, R¹ is

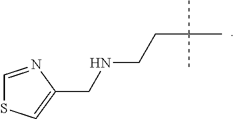

In a further embodiment, R² is

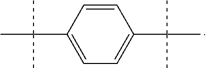

In one embodiment of a compound of Formula (II), or a pharmaceutically acceptable salt or deuterated form thereof, R¹ is

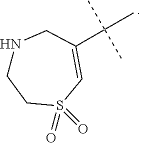

In a further embodiment, R² is

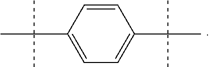

In one embodiment of a compound of Formula (II), or a pharmaceutically acceptable salt or deuterated form thereof, R¹ is

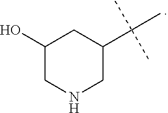

In a further embodiment, R² is

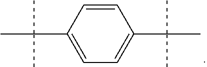

In one embodiment of a compound of Formula (II), or a pharmaceutically acceptable salt or deuterated form thereof, R¹ is

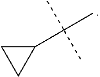

In a further embodiment, R² is

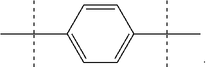

In one embodiment of a compound of Formula (II), or a pharmaceutically acceptable salt or deuterated form thereof, R¹ is

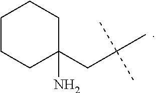

In a further embodiment, R² is

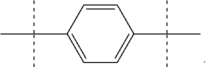

In one embodiment of a compound of Formula (II), or a pharmaceutically acceptable salt or deuterated form thereof, R¹ is

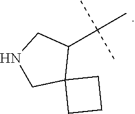

In a further embodiment, R² is

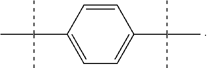

In one embodiment of a compound of Formula (II), or a pharmaceutically acceptable salt or deuterated form thereof, R¹ is

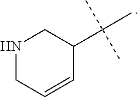

In a further embodiment, R² is

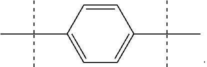

In one embodiment of a compound of Formula (II), or a pharmaceutically acceptable salt or deuterated form thereof, R¹ is

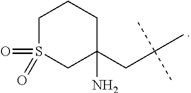

In a further embodiment, R² is

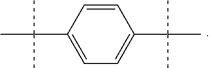

In one embodiment of a compound of Formula (II), or a pharmaceutically acceptable salt thereof, R¹ is

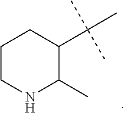

In a further embodiment, R² is

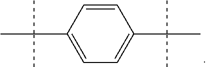

In one embodiment of a compound of Formula (II), or a pharmaceutically acceptable salt or deuterated form thereof, R¹ is

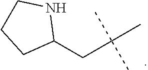

In a further embodiment, R² is

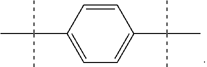

In one embodiment of a compound of Formula (II), or a pharmaceutically acceptable salt or deuterated form thereof, R¹ is

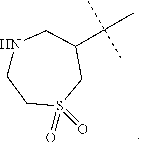

In a further embodiment, R² is

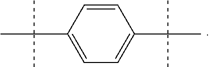

In one embodiment of a compound of Formula (II), or a pharmaceutically acceptable salt or deuterated form thereof, R¹ is

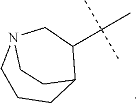

In a further embodiment, R² is

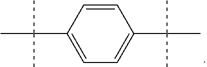

In one embodiment of a compound of Formula (II), or a pharmaceutically acceptable salt or deuterated form thereof, R¹ is

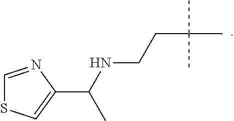

In a further embodiment, R² is

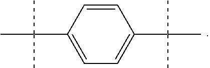

In one embodiment of a compound of Formula (II), or a pharmaceutically acceptable salt or deuterated form thereof, R¹ is

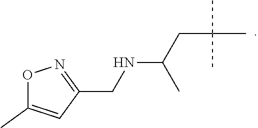

In a further embodiment, R² is

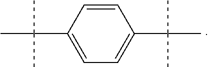

In one embodiment of a compound of Formula (II), L is

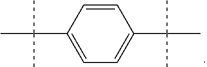

In another embodiment, L is

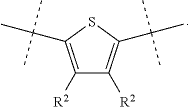

In one embodiment of a compound of Formula (II), L is one of the following:

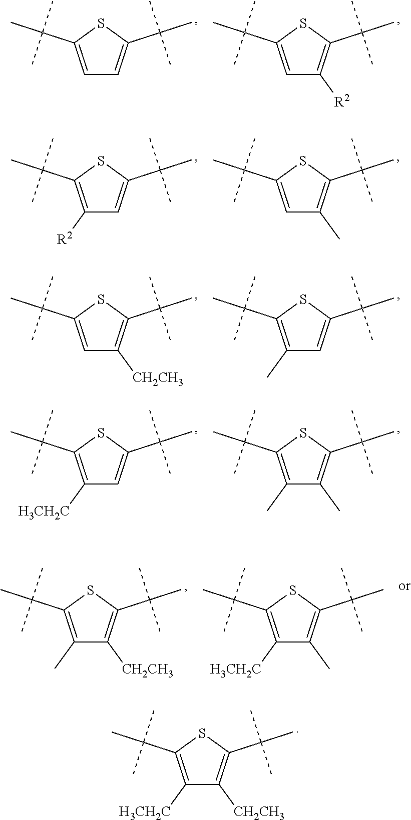

In one embodiment of a compound of Formula (II), L is

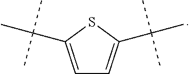

In another embodiment, L is

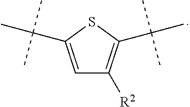

In yet another embodiment L is

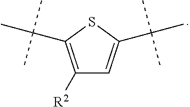

In one embodiment of a compound of Formula (II), L is one of the following:

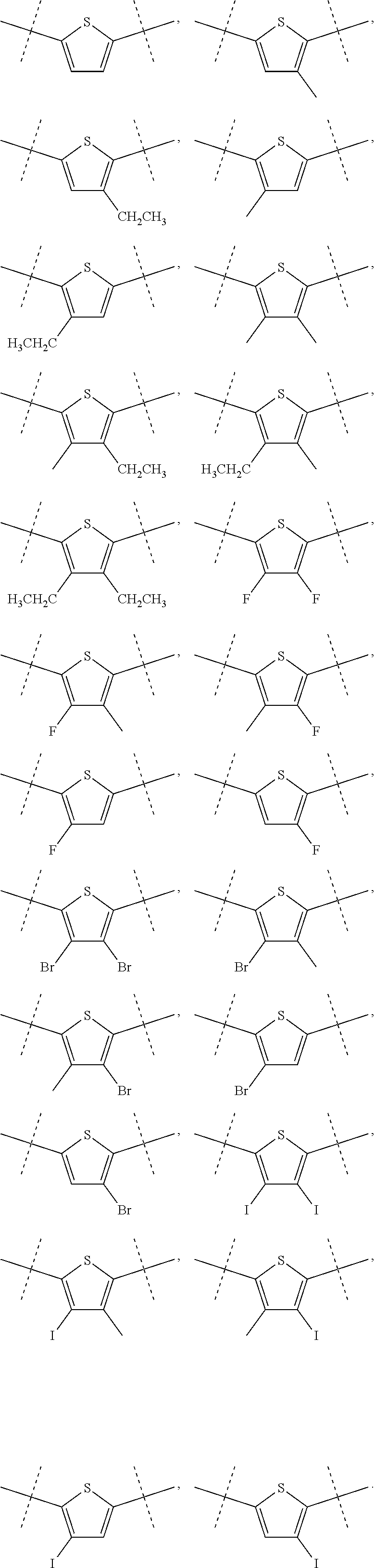

In embodiments of a compound of Formula (II), or a pharmaceutically acceptable salt or deuterated form thereof, the compound is one or more selected from Table 8.

TABLE 8
Representative compounds of Formula (II).

| Compound | Structure |
|---|---|
| 198. | 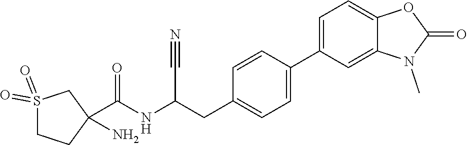 [SMILES: Cn1c(=O)oc2ccc(-c3ccc(CC(C#N)NC(=O)C4(N)CCS(=O)(=O)C4)cc3)cc21] |
| 199. | 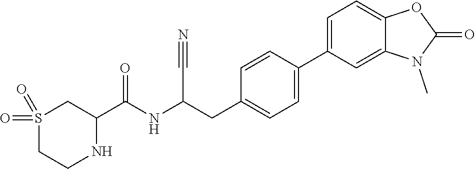 [SMILES: Cn1c(=O)oc2ccc(-c3ccc(CC(C#N)NC(=O)C4CS(=O)(=O)CCN4)cc3)cc21] |
| 200. | 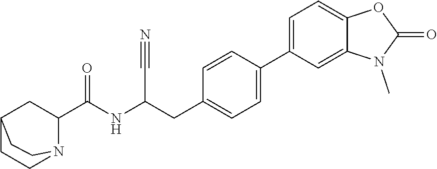 [SMILES: Cn1c(=O)oc2ccc(-c3ccc(CC(C#N)NC(=O)C4CC5CCN4CC5)cc3)cc21] |
| 201. | 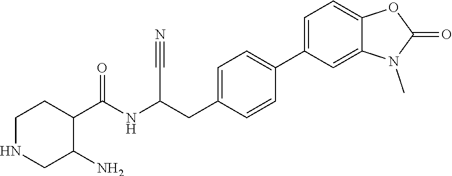 [SMILES: Cn1c(=O)oc2ccc(-c3ccc(CC(C#N)NC(=O)C4CCNCC4N)cc3)cc21] |
| 202. | 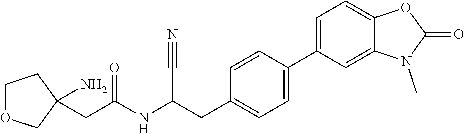 [SMILES: Cn1c(=O)oc2ccc(-c3ccc(CC(C#N)NC(=O)CC4(N)CCOC4)cc3)cc21] |
| 203. | 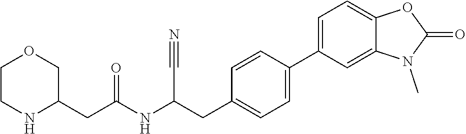 [SMILES: Cn1c(=O)oc2ccc(-c3ccc(CC(C#N)NC(=O)CC4COCCN4)cc3)cc21] |
| 204. | 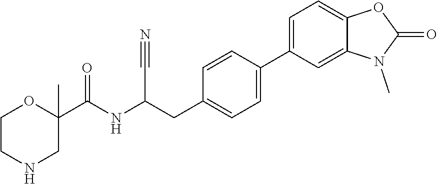 [SMILES: Cn1c(=O)oc2ccc(-c3ccc(CC(C#N)NC(=O)C4(C)CNCCO4)cc3)cc21] |
| 205. | 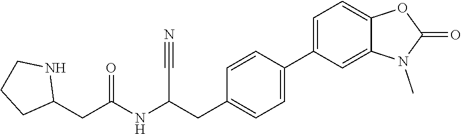 [SMILES: Cn1c(=O)oc2ccc(-c3ccc(CC(C#N)NC(=O)CC4CCCN4)cc3)cc21] |
| 206. | 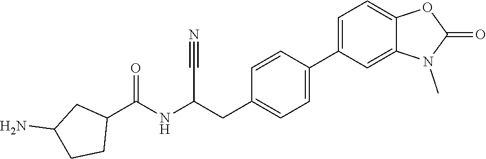 [SMILES: Cn1c(=O)oc2ccc(-c3ccc(CC(C#N)NC(=O)C4CCC(N)C4)cc3)cc21] |
| 207. | 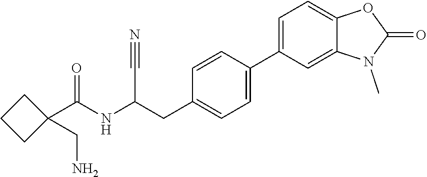 [SMILES: Cn1c(=O)oc2ccc(-c3ccc(CC(C#N)NC(=O)C4(CN)CCC4)cc3)cc21] |
| 208. | 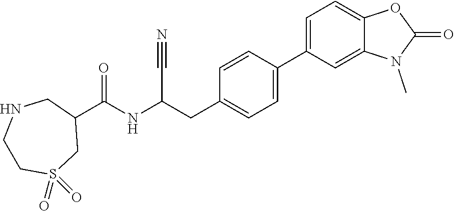 [SMILES: Cn1c(=O)oc2ccc(-c3ccc(CC(C#N)NC(=O)C4CNCCS(=O)(=O)C4)cc3)cc21] |
| 209. | 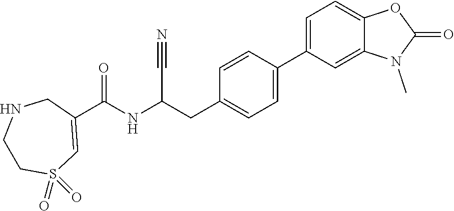 [SMILES: Cn1c(=O)oc2ccc(-c3ccc(CC(C#N)NC(=O)C4=CS(=O)(=O)CCNC4)cc3)cc21] |
| 210. | 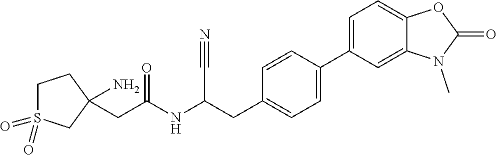 [SMILES: Cn1c(=O)oc2ccc(-c3ccc(CC(C#N)NC(=O)CC4(N)CCS(=O)(=O)C4)cc3)cc21] |
| 211. | 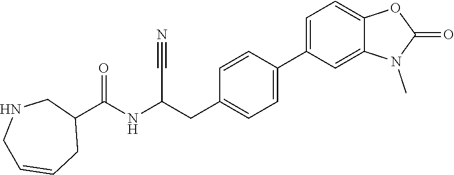 [SMILES: Cn1c(=O)oc2ccc(-c3ccc(CC(C#N)NC(=O)C4CC=CCNC4)cc3)cc21] |
| 212. | 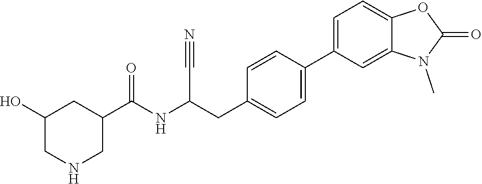 [SMILES: Cn1c(=O)oc2ccc(-c3ccc(CC(C#N)NC(=O)C4CNCC(O)C4)cc3)cc21] |
| 213. | 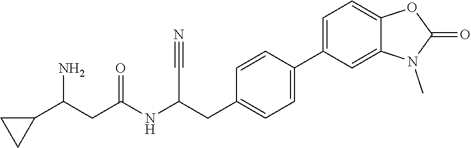 [SMILES: Cn1c(=O)oc2ccc(-c3ccc(CC(C#N)NC(=O)CC(N)C4CC4)cc3)cc21] |
| 214. | 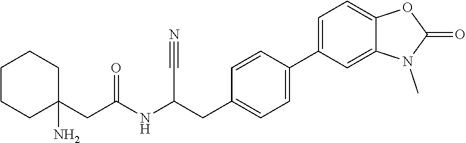 [SMILES: Cn1c(=O)oc2ccc(-c3ccc(CC(C#N)NC(=O)CC4(N)CCCCC4)cc3)cc21] |
| 215. | 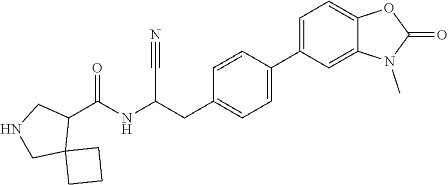 [SMILES: Cn1c(=O)oc2ccc(-c3ccc(CC(C#N)NC(=O)C4CNCC45CCC5)cc3)cc21] |
| 216. | 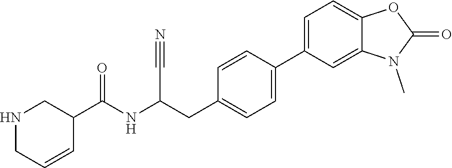 [SMILES: Cn1c(=O)oc2ccc(-c3ccc(CC(C#N)NC(=O)C4C=CCNC4)cc3)cc21] |
| 217. | 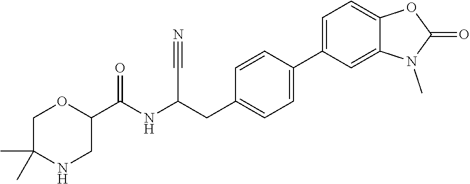 [SMILES: Cn1c(=O)oc2ccc(-c3ccc(CC(C#N)NC(=O)C4CNC(C)(C)CO4)cc3)cc21] |
| 218. | 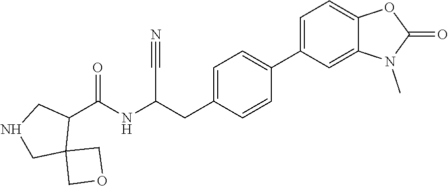 [SMILES: Cn1c(=O)oc2ccc(-c3ccc(CC(C#N)NC(=O)C4CNCC45COC5)cc3)cc21] |
| 219. | 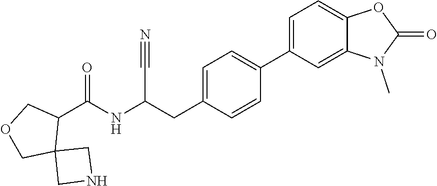 [SMILES: Cn1c(=O)oc2ccc(-c3ccc(CC(C#N)NC(=O)C4COCC45CNC5)cc3)cc21] |
| 220. | 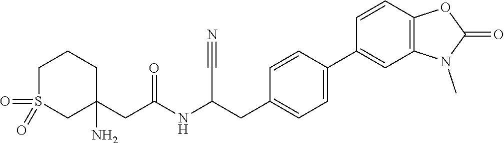 [SMILES: Cn1c(=O)oc2ccc(-c3ccc(CC(C#N)NC(=O)CC4(N)CCCS(=O)(=O)C4)cc3)cc21] |
| 221. | 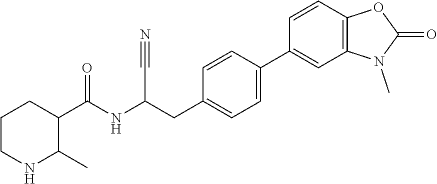 [SMILES: CC1NCCCC1C(=O)NC(C#N)Cc1ccc(-c2ccc3oc(=O)n(C)c3c2)cc1] |
| 222. | 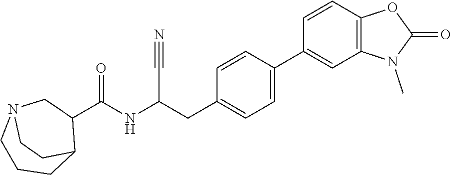 [SMILES: Cn1c(=O)oc2ccc(-c3ccc(CC(C#N)NC(=O)C4CN5CCCC4CC5)cc3)cc21] |
| 223. | 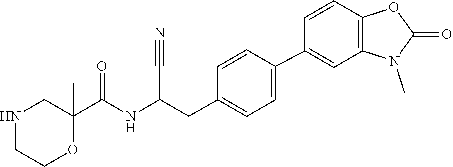 [SMILES: Cn1c(=O)oc2ccc(-c3ccc(CC(C#N)NC(=O)C4(C)CNCCO4)cc3)cc21] |

In embodiments of a compound of Formula (II), or a pharmaceutically acceptable salt or deuterated form thereof, the compound is one or more selected from Table 9.

TABLE 9
Representative compounds of Formula (II).

| Compound | Structure |
|---|---|
| 224. | 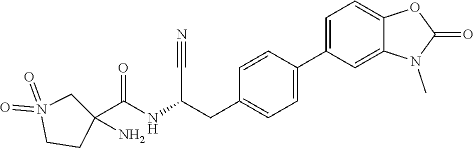 |
| 225. | 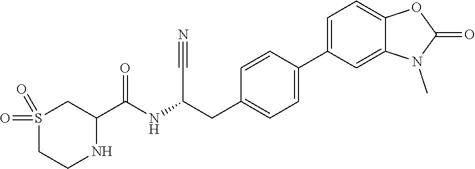 [SMILES: Cn1c(=O)oc2ccc(-c3ccc(C[C@@H](C#N)NC(=O)C4CS(=O)(=O)CCN4)cc3)cc21] |
| 226. | 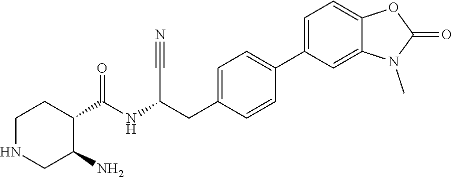 [SMILES: Cn1c(=O)oc2ccc(-c3ccc(C[C@@H](C#N)NC(=O)[C@H]4CCNC[C@@H]4N)cc3)cc21] |
| 227. | 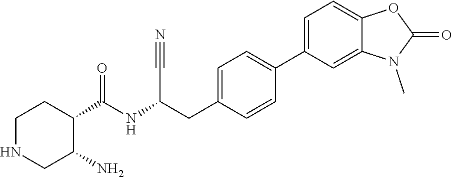 [SMILES: Cn1c(=O)oc2ccc(-c3ccc(C[C@@H](C#N)NC(=O)[C@H]4CCNC[C@H]4N)cc3)cc21] |
| 228. | 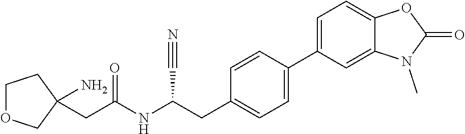 [SMILES: Cn1c(=O)oc2ccc(-c3ccc(C[C@@H](C#N)NC(=O)CC4(N)CCOC4)cc3)cc21] |
| 229. | 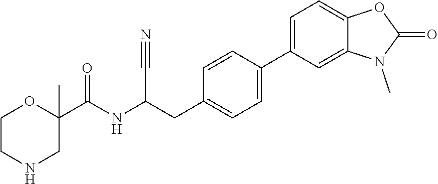 [SMILES: Cn1c(=O)oc2ccc(-c3ccc(CC(C#N)NC(=O)C4(C)CNCCO4)cc3)cc21] |
| 230. | 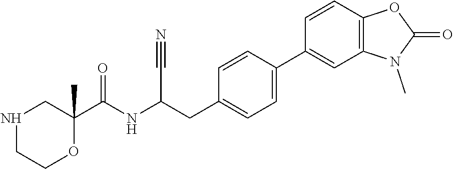 [SMILES: Cn1c(=O)oc2ccc(-c3ccc(CC(C#N)NC(=O)[C@@]4(C)CNCCO4)cc3)cc21] |
| 231. | 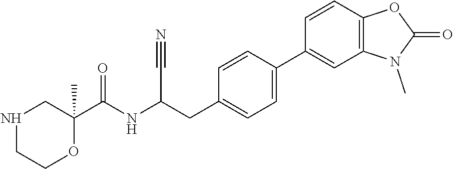 [SMILES: Cn1c(=O)oc2ccc(-c3ccc(CC(C#N)NC(=O)[C@]4(C)CNCCO4)cc3)cc21] |
| 232. | 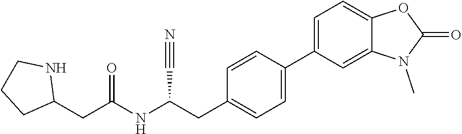 [SMILES: Cn1c(=O)oc2ccc(-c3ccc(C[C@@H](C#N)NC(=O)CC4CCCN4)cc3)cc21] |
| 233. | 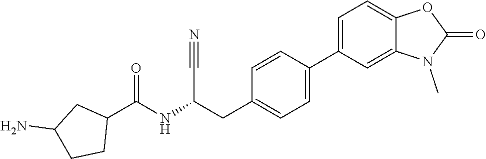 [SMILES: Cn1c(=O)oc2ccc(-c3ccc(C[C@@H](C#N)NC(=O)C4CCC(N)C4)cc3)cc21] |
| 234. | 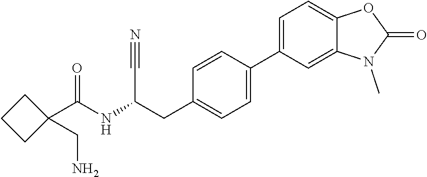 [SMILES: Cn1c(=O)oc2ccc(-c3ccc(C[C@@H](C#N)NC(=O)C4(CN)CCC4)cc3)cc21] |
| 235. | 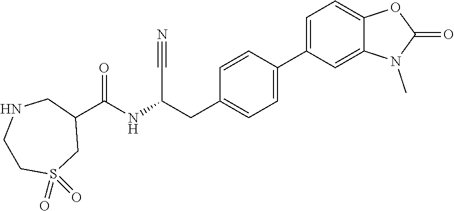 [SMILES: Cn1c(=O)oc2ccc(-c3ccc(C[C@@H](C#N)NC(=O)C4CNCCS(=O)(=O)C4)cc3)cc21] |
| 236. | 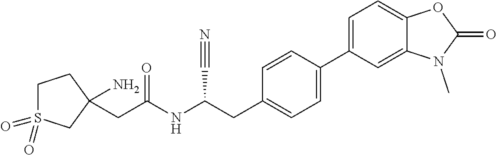 [SMILES: Cn1c(=O)oc2ccc(-c3ccc(C[C@@H](C#N)NC(=O)CC4(N)CCS(=O)(=O)C4)cc3)cc21] |
| 237. | 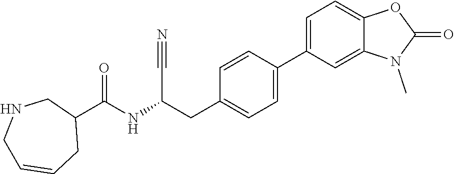 [SMILES: Cn1c(=O)oc2ccc(-c3ccc(C[C@@H](C#N)NC(=O)C4CC=CCNC4)cc3)cc21] |
| 238. | 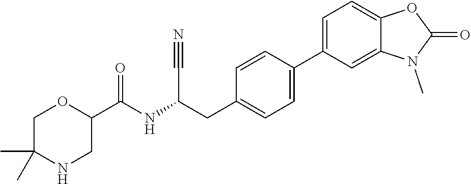 [SMILES: Cn1c(=O)oc2ccc(-c3ccc(C[C@@H](C#N)NC(=O)C4CNC(C)(C)CO4)cc3)cc21] |
| 239. | 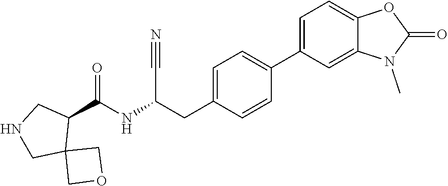 [SMILES: Cn1c(=O)oc2ccc(-c3ccc(C[C@@H](C#N)NC(=O)[C@@H]4CNCC45COC5)cc3)cc21] |
| 240. | 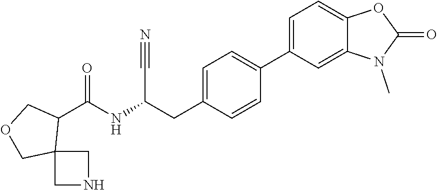 [SMILES: Cn1c(=O)oc2ccc(-c3ccc(C[C@@H](C#N)NC(=O)C4COCC45CNC5)cc3)cc21] |
| 241. | 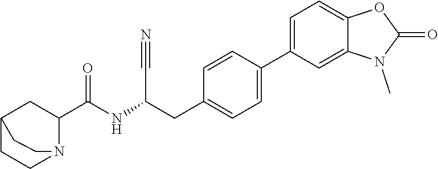 [SMILES: Cn1c(=O)oc2ccc(-c3ccc(C[C@@H](C#N)NC(=O)C4CC5CCN4CC5)cc3)cc21] |
| 242. | 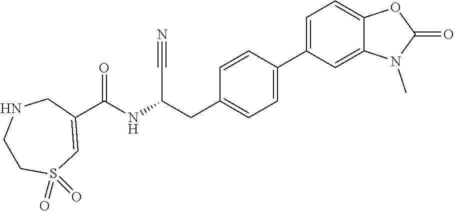 [SMILES: Cn1c(=O)oc2ccc(-c3ccc(C[C@@H](C#N)NC(=O)C4=CS(=O)(=O)CCNC4)cc3)cc21] |
| 243. | 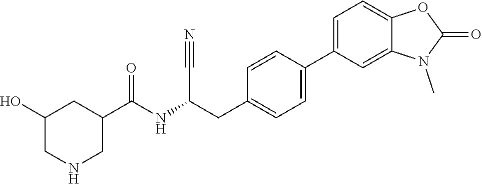 [SMILES: Cn1c(=O)oc2ccc(-c3ccc(C[C@@H](C#N)NC(=O)C4CNCC(O)C4)cc3)cc21] |
| 244. | 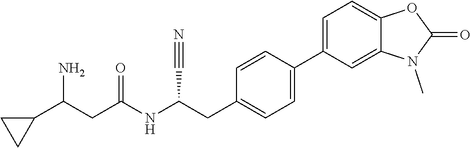 [SMILES: Cn1c(=O)oc2ccc(-c3ccc(C[C@@H](C#N)NC(=O)CC(N)C4CC4)cc3)cc21] |
| 245. | 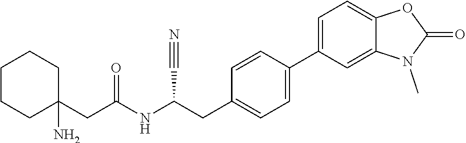 [SMILES: Cn1c(=O)oc2ccc(-c3ccc(C[C@@H](C#N)NC(=O)CC4(N)CCCCC4)cc3)cc21] |
| 246. | 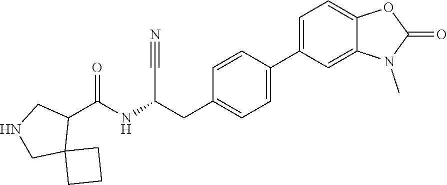 [SMILES: Cn1c(=O)oc2ccc(-c3ccc(C[C@@H](C#N)NC(=O)C4CNCC45CCC5)cc3)cc21] |
| 247. | 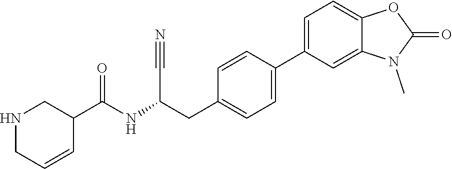 [SMILES: Cn1c(=O)oc2ccc(-c3ccc(C[C@@H](C#N)NC(=O)C4C=CCNC4)cc3)cc21] |
| 248. | 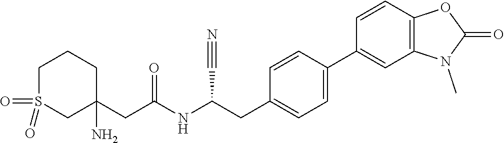 [SMILES: Cn1c(=O)oc2ccc(-c3ccc(C[C@@H](C#N)NC(=O)CC4(N)CCCS(=O)(=O)C4)cc3)cc21] |
| 249. | 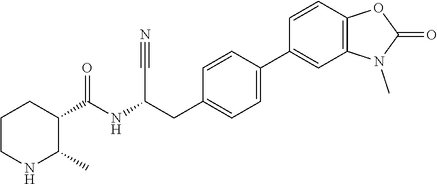 [SMILES: C[C@@H]1NCCC[C@@H]1C(=O)N[C@H](C#N)Cc1ccc(-c2ccc3oc(=O)n(C)c3c2)cc1] |
| 250. | 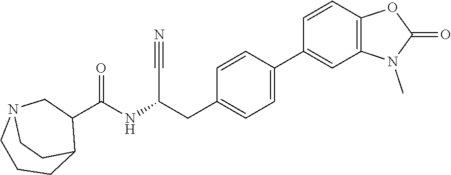 [SMILES: Cn1c(=O)oc2ccc(-c3ccc(C[C@@H](C#N)NC(=O)C4CN5CCCC4CC5)cc3)cc21] |
| 294 | 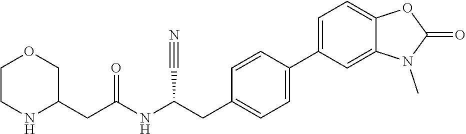 [SMILES: Cn1c(=O)oc2ccc(-c3ccc(C[C@@H](C#N)NC(=O)CC4COCCN4)cc3)cc21] |
| 249A | 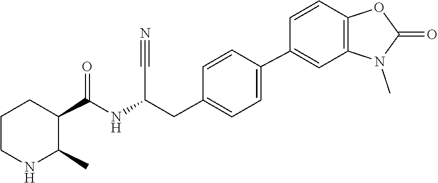 [SMILES: C[C@H]1NCCC[C@H]1C(=O)N[C@H](C#N)Cc1ccc(-c2ccc3oc(=O)n(C)c3c2)cc1] |
| 295 | 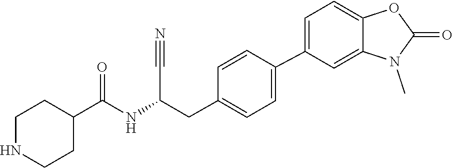 [SMILES: Cn1c(=O)oc2ccc(-c3ccc(C[C@@H](C#N)NC(=O)C4CCNCC4)cc3)cc21] |
| 308 | 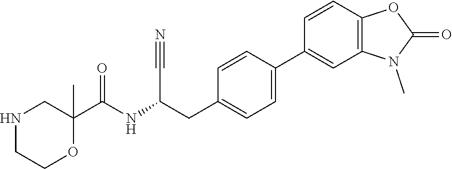 [SMILES: Cn1c(=O)oc2ccc(-c3ccc(C[C@@H](C#N)NC(=O)C4(C)CNCCO4)cc3)cc21] |
| 308A | 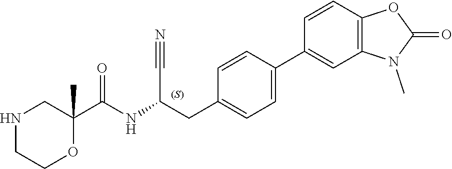 |
| 308B | 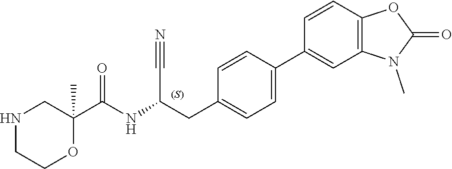 |
| | 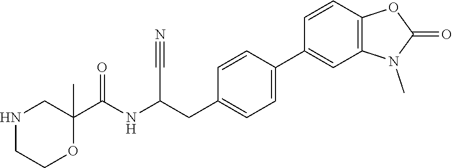 [SMILES: Cn1c(=O)oc2ccc(-c3ccc(CC(C#N)NC(=O)C4(C)CNCCO4)cc3)cc21] |
| 257 | 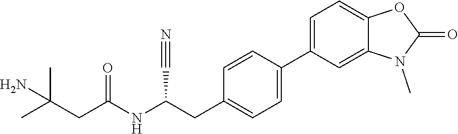 [SMILES: Cn1c(=O)oc2ccc(-c3ccc(C[C@@H](C#N)NC(=O)CC(C)(C)N)cc3)cc21] |
| 257a | 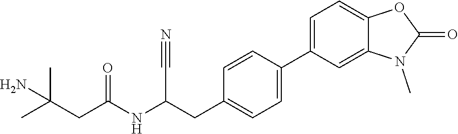 [SMILES: Cn1c(=O)oc2ccc(-c3ccc(CC(C#N)NC(=O)CC(C)(C)N)cc3)cc21] |
| 258 | 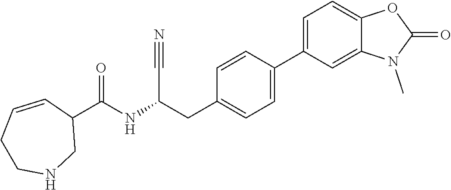 [SMILES: Cn1c(=O)oc2ccc(-c3ccc(C[C@@H](C#N)NC(=O)C4C=CCCNC4)cc3)cc21] |
| 258a | 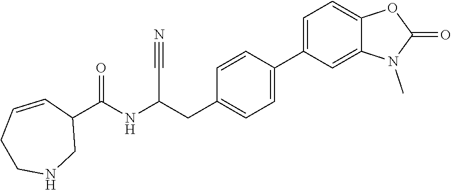 [SMILES: Cn1c(=O)oc2ccc(-c3ccc(CC(C#N)NC(=O)C4C=CCCNC4)cc3)cc21] |
| 260 | 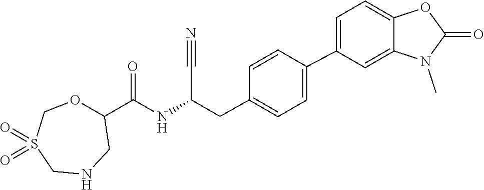 [SMILES: Cn1c(=O)oc2ccc(-c3ccc(C[C@@H](C#N)NC(=O)C4CNCS(=O)(=O)CO4)cc3)cc21] |
| 260a | 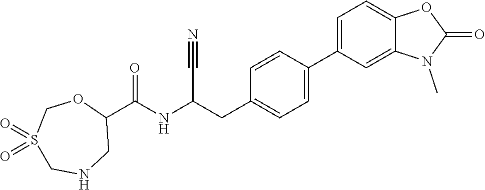 [SMILES: Cn1c(=O)oc2ccc(-c3ccc(CC(C#N)NC(=O)C4CNCS(=O)(=O)CO4)cc3)cc21] |
| 262 | 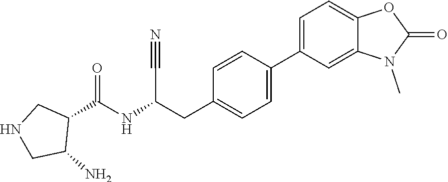 [SMILES: Cn1c(=O)oc2ccc(-c3ccc(C[C@@H](C#N)NC(=O)[C@H]4CNC[C@H]4N)cc3)cc21] |
| 262a | 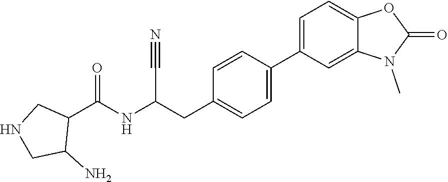 [SMILES: Cn1c(=O)oc2ccc(-c3ccc(CC(C#N)NC(=O)C4CNCC4N)cc3)cc21] |
| 263 | 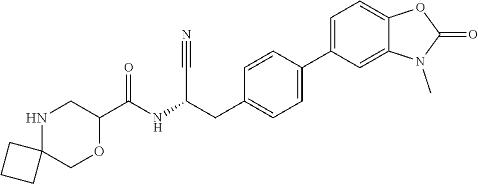 [SMILES: Cn1c(=O)oc2ccc(-c3ccc(C[C@@H](C#N)NC(=O)C4CNC5(CCC5)CO4)cc3)cc21] |
| 263a | 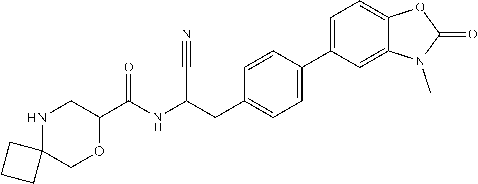 [SMILES: Cn1c(=O)oc2ccc(-c3ccc(CC(C#N)NC(=O)C4CNC5(CCC5)CO4)cc3)cc21] |
| 264 | 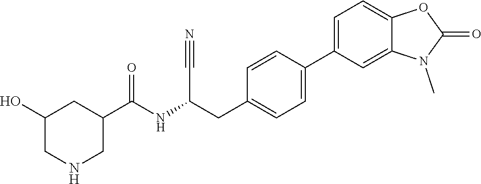 [SMILES: Cn1c(=O)oc2ccc(-c3ccc(C[C@@H](C#N)NC(=O)C4CNCC(O)C4)cc3)cc21] |
| 264a | 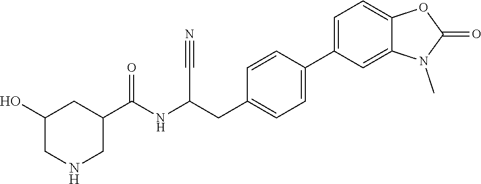 [SMILES: Cn1c(=O)oc2ccc(-c3ccc(CC(C#N)NC(=O)C4CNCC(O)C4)cc3)cc21] |
| 264b | 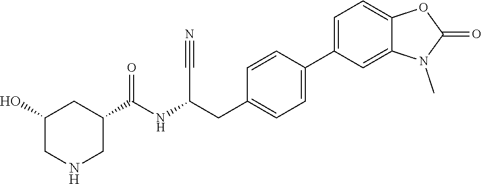 [SMILES: Cn1c(=O)oc2ccc(-c3ccc(C[C@@H](C#N)NC(=O)[C@@H]4CNC[C@H](O)C4)cc3)cc21] |
| 264c | 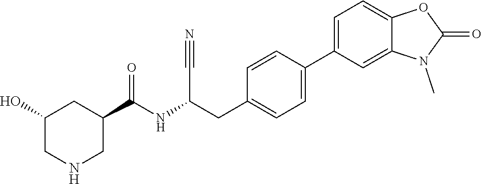 [SMILES: Cn1c(=O)oc2ccc(-c3ccc(C[C@@H](C#N)NC(=O)[C@H]4CNC[C@H](O)C4)cc3)cc21] |
| 264d | 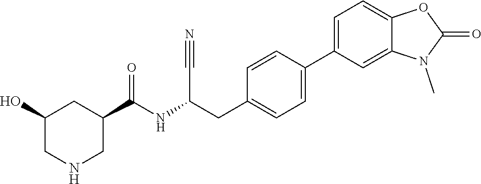 [SMILES: Cn1c(=O)oc2ccc(-c3ccc(C[C@@H](C#N)NC(=O)[C@H]4CNC[C@@H](O)C4)cc3)cc21] |
| 264e | 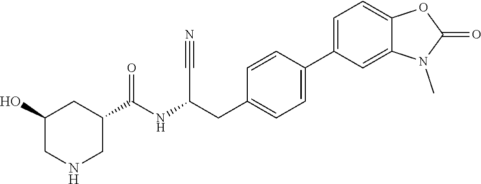 [SMILES: Cn1c(=O)oc2ccc(-c3ccc(C[C@@H](C#N)NC(=O)[C@@H]4CNC[C@@H](O)C4)cc3)cc21] |
| 265 | 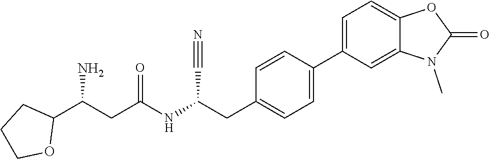 [SMILES: Cn1c(=O)oc2ccc(-c3ccc(C[C@@H](C#N)NC(=O)C[C@@H](N)C4CCCO4)cc3)cc21] |
| 265A | 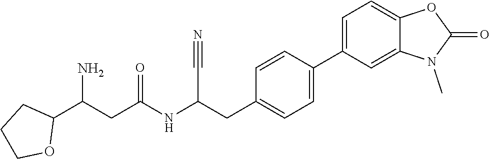 [SMILES: Cn1c(=O)oc2ccc(-c3ccc(CC(C#N)NC(=O)CC(N)C4CCCO4)cc3)cc21] |
| 266 | 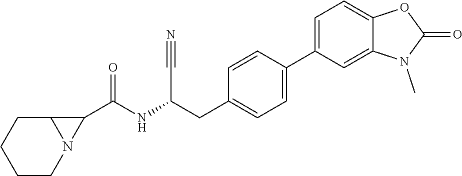 [SMILES: Cn1c(=O)oc2ccc(-c3ccc(C[C@@H](C#N)NC(=O)C4C5CCCCN54)cc3)cc21] |
| 266 | 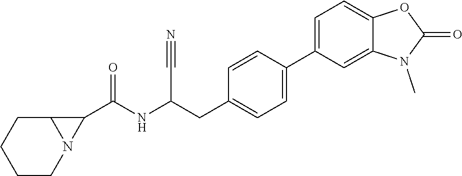 [SMILES: Cn1c(=O)oc2ccc(-c3ccc(CC(C#N)NC(=O)C4C5CCCCN54)cc3)cc21] |
| 267 | 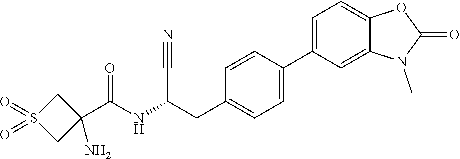 [SMILES: Cn1c(=O)oc2ccc(-c3ccc(C[C@@H](C#N)NC(=O)C4(N)CS(=O)(=O)C4)cc3)cc21] |
| 267A | 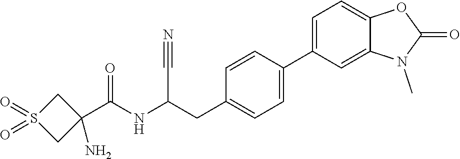 [SMILES: Cn1c(=O)oc2ccc(-c3ccc(CC(C#N)NC(=O)C4(N)CS(=O)(=O)C4)cc3)cc21] |
| 268 | 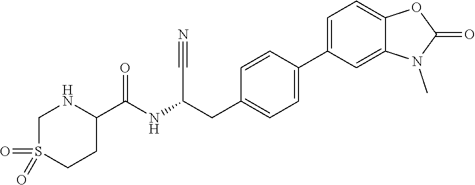 [SMILES: Cn1c(=O)oc2ccc(-c3ccc(C[C@@H](C#N)NC(=O)C4CCS(=O)(=O)CN4)cc3)cc21] |
| 268A | 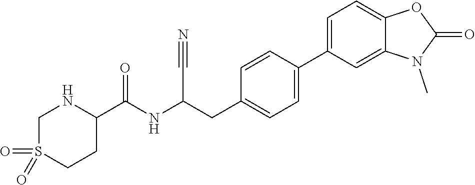 [SMILES: Cn1c(=O)oc2ccc(-c3ccc(CC(C#N)NC(=O)C4CCS(=O)(=O)CN4)cc3)cc21] |
| 269 | 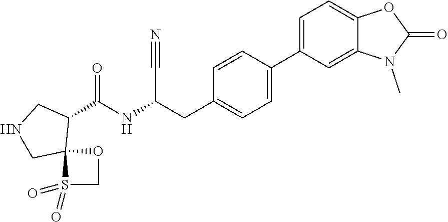 [SMILES: Cn1c(=O)oc2ccc(-c3ccc(C[C@@H](C#N)NC(=O)[C@H]4CNC[C@@]45OCS5(=O)=O)cc3)cc21] |
| 269A | 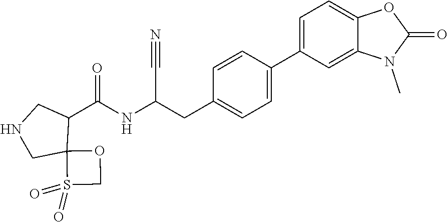 [SMILES: Cn1c(=O)oc2ccc(-c3ccc(CC(C#N)NC(=O)C4CNCC45OCS5(=O)=O)cc3)cc21] |
| 270 | 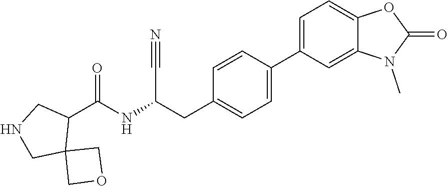 [SMILES: Cn1c(=O)oc2ccc(-c3ccc(C[C@@H](C#N)NC(=O)C4CNCC45COC5)cc3)cc21] |
| 271 | 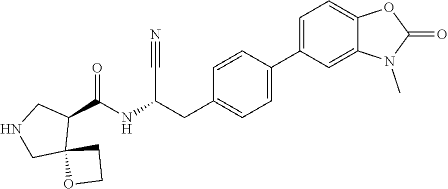 [SMILES: Cn1c(=O)oc2ccc(-c3ccc(C[C@@H](C#N)NC(=O)[C@@H]4CNC[C@]45CCO5)cc3)cc21] |
| 271A | 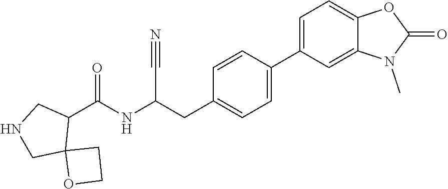 [SMILES: Cn1c(=O)oc2ccc(-c3ccc(CC(C#N)NC(=O)C4CNCC45CCO5)cc3)cc21] |
| 272 | 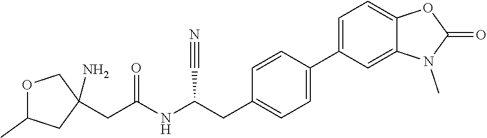 [SMILES: CC1CC(N)(CC(=O)N[C@H](C#N)Cc2ccc(-c3ccc4oc(=O)n(C)c4c3)cc2)CO1] |
| 272A | 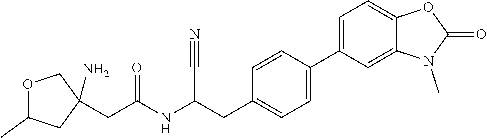 [SMILES: CC1CC(N)(CC(=O)NC(C#N)Cc2ccc(-c3ccc4oc(=O)n(C)c4c3)cc2)CO1] |
| 273 | 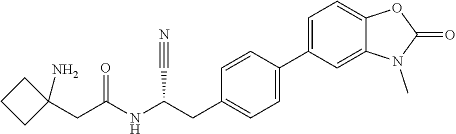 [SMILES: Cn1c(=O)oc2ccc(-c3ccc(C[C@@H](C#N)NC(=O)CC4(N)CCC4)cc3)cc21] |
| 273A | 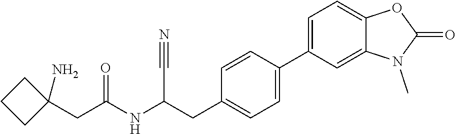 [SMILES: Cn1c(=O)oc2ccc(-c3ccc(CC(C#N)NC(=O)CC4(N)CCC4)cc3)cc21] |
| 274 | 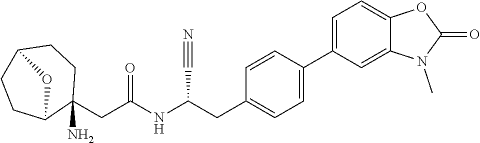 [SMILES: Cn1c(=O)oc2ccc(-c3ccc(C[C@@H](C#N)NC(=O)C[C@@]4(N)CC[C@@H]5CC[C@H]4O5)cc3)cc21] |
| 274A | 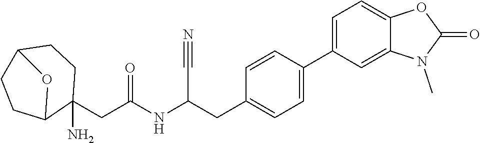 [SMILES: Cn1c(=O)oc2ccc(-c3ccc(CC(C#N)NC(=O)CC4(N)CCC5CCC4O5)cc3)cc21] |
| 275 | 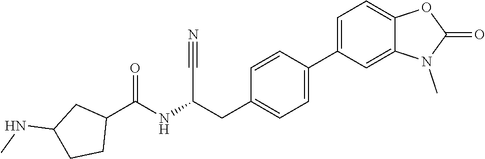 [SMILES: CNC1CCC(C(=O)N[C@H](C#N)Cc2ccc(-c3ccc4oc(=O)n(C)c4c3)cc2)C1] |
| 275A | 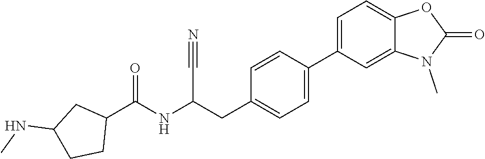 [SMILES: CNC1CCC(C(=O)NC(C#N)Cc2ccc(-c3ccc4oc(=O)n(C)c4c3)cc2)C1] |
| 276 | 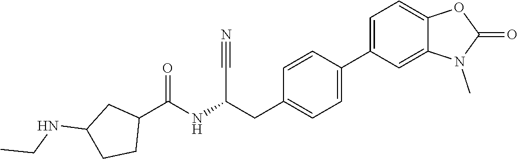 [SMILES: CCNC1CCC(C(=O)N[C@H](C#N)Cc2ccc(-c3ccc4oc(=O)n(C)c4c3)cc2)C1] |
| 276A | 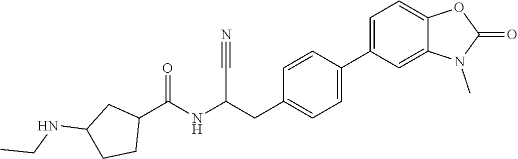 [SMILES: CCNC1CCC(C(=O)NC(C#N)Cc2ccc(-c3ccc4oc(=O)n(C)c4c3)cc2)C1] |
| 277 | 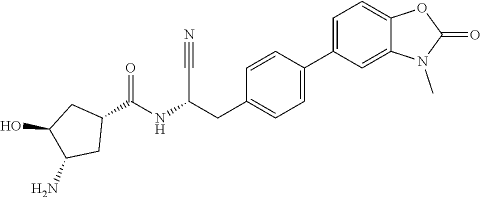 [SMILES: Cn1c(=O)oc2ccc(-c3ccc(C[C@@H](C#N)NC(=O)[C@@H]4C[C@H](N)[C@@H](O)C4)cc3)cc21] |
| 277A | 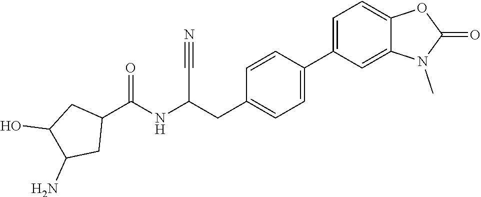 [SMILES: Cn1c(=O)oc2ccc(-c3ccc(CC(C#N)NC(=O)C4CC(N)C(O)C4)cc3)cc21] |
| 278 | 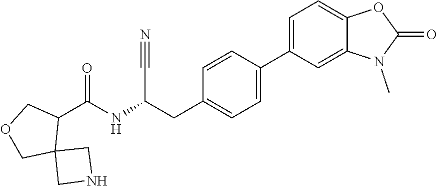 [SMILES: Cn1c(=O)oc2ccc(-c3ccc(C[C@@H](C#N)NC(=O)C4COCC45CNC5)cc3)cc21] |
| 278A | 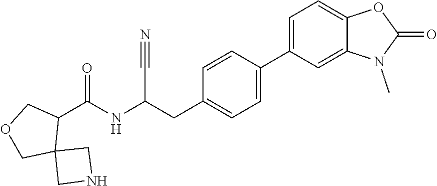 [SMILES: Cn1c(=O)oc2ccc(-c3ccc(CC(C#N)NC(=O)C4COCC45CNC5)cc3)cc21] |
| 279 | 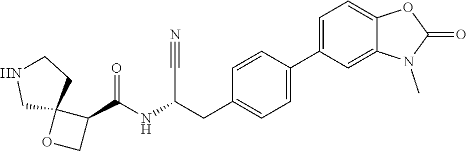 [SMILES: Cn1c(=O)oc2ccc(-c3ccc(C[C@@H](C#N)NC(=O)[C@H]4CO[C@@]45CCNC5)cc3)cc21] |
| 279a | 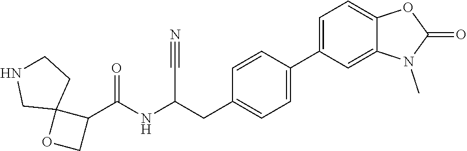 [SMILES: Cn1c(=O)oc2ccc(-c3ccc(CC(C#N)NC(=O)C4COC45CCNC5)cc3)cc21] |

### Formula (III)

In yet another aspect, a DPP1 inhibitor provided herein is a sulfonamide compound of Formula (III), or a pharmaceutically acceptable salt or deuterated form thereof:

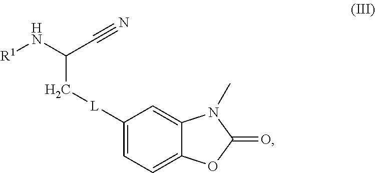

wherein,
R¹ is

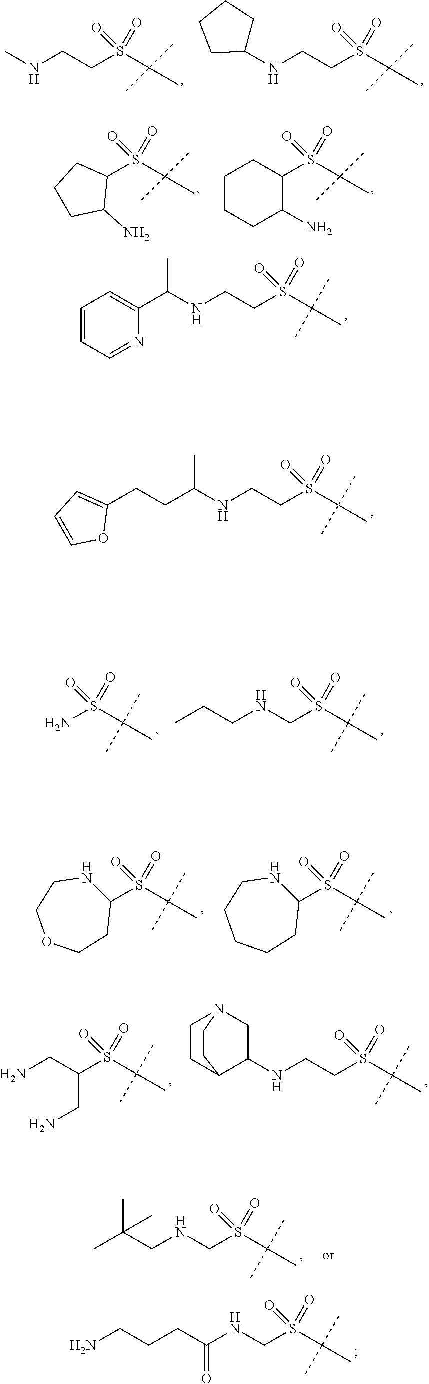

L is

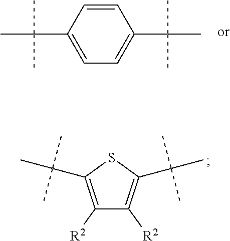

and
each R² is independently H, F, Cl, Br, I or C₁-C₆ alkyl.

In one embodiment of a compound of Formula (III), or a pharmaceutically acceptable salt or deuterated form thereof, R¹ is

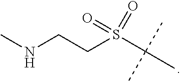

In a further embodiment, L is

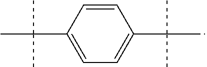

In one embodiment of a compound of Formula (III), or a pharmaceutically acceptable salt or deuterated form thereof, R¹ is

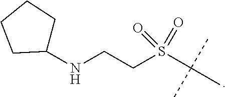

In a further embodiment, L is

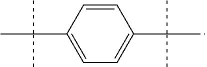

In one embodiment of a compound of Formula (III), or a pharmaceutically acceptable salt or deuterated form thereof, R¹ is

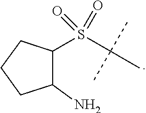

In a further embodiment, L is

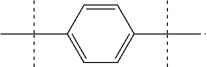

In one embodiment of a compound of Formula (III), or a pharmaceutically acceptable salt or deuterated form thereof, R¹ is

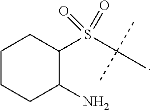

In a further embodiment, L is

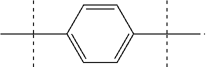

In one embodiment of a compound of Formula (III), or a pharmaceutically acceptable salt or deuterated form thereof, R¹ is

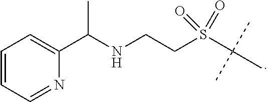

In a further embodiment, L is

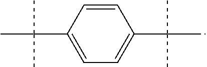

In one embodiment of a compound of Formula (III), or a pharmaceutically acceptable salt or deuterated form thereof, R¹ is

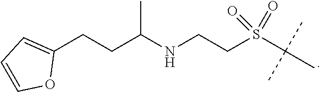

In a further embodiment, L is

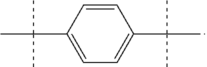

In one embodiment of a compound of Formula (III), or a pharmaceutically acceptable salt or deuterated form thereof, R¹ is

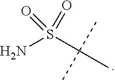

In a further embodiment, L is

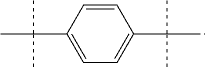

In one embodiment of a compound of Formula (III), or a pharmaceutically acceptable salt or deuterated form thereof, R¹ is

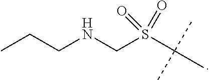

In a further embodiment, L is

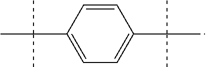

In one embodiment of a compound of Formula (III), or a pharmaceutically acceptable salt or deuterated form thereof, R¹ is

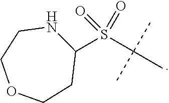

In a further embodiment, L is

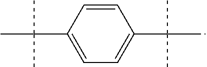

In one embodiment of a compound of Formula (III), or a pharmaceutically acceptable salt or deuterated form thereof, R¹ is

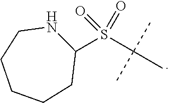

In a further embodiment, L is

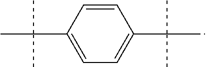

In one embodiment of a compound of Formula (III), or a pharmaceutically acceptable salt or deuterated form thereof, R¹ is

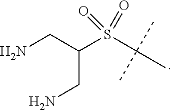

In a further embodiment, L is

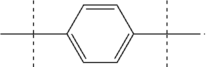

In one embodiment of a compound of Formula (III), or a pharmaceutically acceptable salt or deuterated form thereof, R¹ is

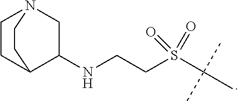

In a further embodiment, L is

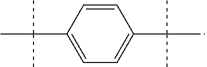

In one embodiment of a compound of Formula (III), or a pharmaceutically acceptable salt or deuterated form thereof, R¹ is

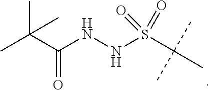

In a further embodiment, L is

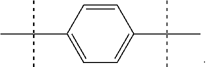

In one embodiment of a compound of Formula (III), or a pharmaceutically acceptable salt or deuterated form thereof, R¹ is

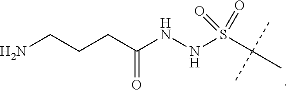

In a further embodiment, L is

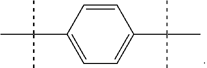

In one embodiment of a compound of Formula (III), or a pharmaceutically acceptable salt or deuterated form thereof, L is

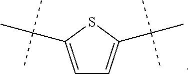

In another embodiment, L is

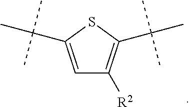

In yet another embodiment L is

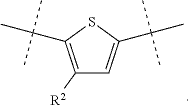

In embodiments of a compound of Formula (III), or a pharmaceutically acceptable salt or deuterated form thereof, the compound is one or more selected from Table 10.

TABLE 10
Table 10. Representative compounds of Formula (III).

| compound | Structure |
|---|---|
| 251. | 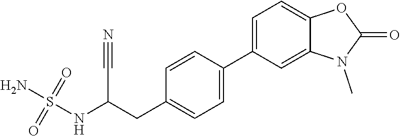 [SMILES: Cn1c(=O)oc2ccc(-c3ccc(CC(C#N)NS(N)(=O)=O)cc3)cc21] |
| 252. | 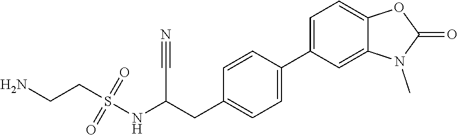 [SMILES: Cn1c(=O)oc2ccc(-c3ccc(CC(C#N)NS(=O)(=O)CCN)cc3)cc21] |
| 253. | 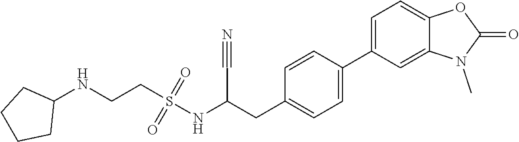 [SMILES: Cn1c(=O)oc2ccc(-c3ccc(CC(C#N)NS(=O)(=O)CCNC4CCCC4)cc3)cc21] |

In embodiments of a compound of Formula (III), or a pharmaceutically acceptable salt or deuterated form thereof, the compound is one or more selected from Table 11.

TABLE 11
Representative compounds of Formula (III).

| compound | Structure |
|---|---|
| 254. | 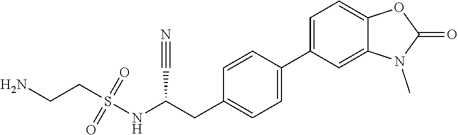 [SMILES: Cn1c(=O)oc2ccc(-c3ccc(C[C@@H](C#N)NS(=O)(=O)CCN)cc3)cc21] |
| 255. | 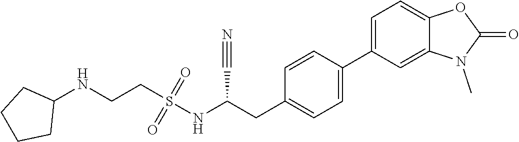 [SMILES: Cn1c(=O)oc2ccc(-c3ccc(C[C@@H](C#N)NS(=O)(=O)CCNC4CCCC4)cc3)cc21] |
| 256. | 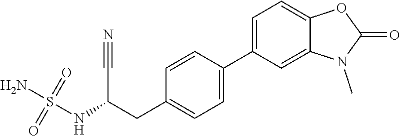 [SMILES: Cn1c(=O)oc2ccc(-c3ccc(C[C@@H](C#N)NS(N)(=O)=O)cc3)cc21] |
| 280 | 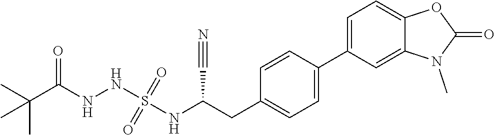 [SMILES: Cn1c(=O)oc2ccc(-c3ccc(C[C@@H](C#N)NS(=O)(=O)NNC(=O)C(C)(C)C)cc3)cc21] |
| 280A | 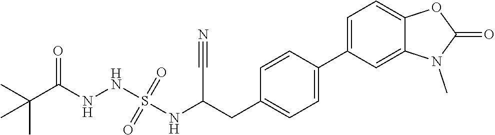 [SMILES: Cn1c(=O)oc2ccc(-c3ccc(CC(C#N)NS(=O)(=O)NNC(=O)C(C)(C)C)cc3)cc21] |
| 281 | 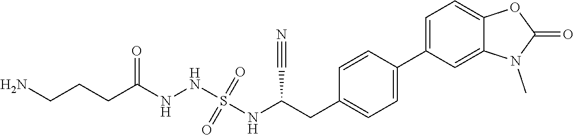 [SMILES: Cn1c(=O)oc2ccc(-c3ccc(C[C@@H](C#N)NS(=O)(=O)NNC(=O)CCCN)cc3)cc21] |
| 281A | 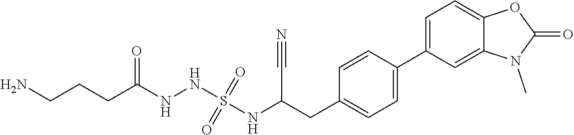 [SMILES: Cn1c(=O)oc2ccc(-c3ccc(CC(C#N)NS(=O)(=O)NNC(=O)CCCN)cc3)cc21] |
| 282 | 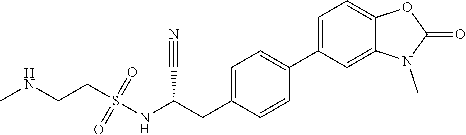 [SMILES: CNCCS(=O)(=O)N[C@H](C#N)Cc1ccc(-c2ccc3oc(=O)n(C)c3c2)cc1] |
| 282A | 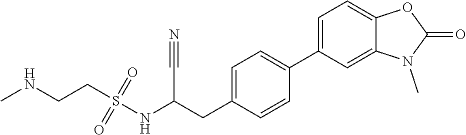 [SMILES: CNCCS(=O)(=O)NC(C#N)Cc1ccc(-c2ccc3oc(=O)n(C)c3c2)cc1] |
| 283 | 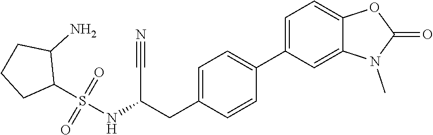 [SMILES: Cn1c(=O)oc2ccc(-c3ccc(C[C@@H](C#N)NS(=O)(=O)C4CCCC4N)cc3)cc21] |
| 283A | 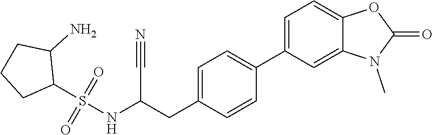 [SMILES: Cn1c(=O)oc2ccc(-c3ccc(CC(C#N)NS(=O)(=O)C4CCCC4N)cc3)cc21] |
| 284 | 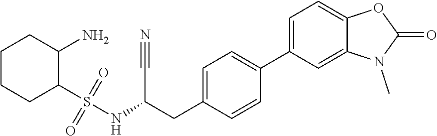 [SMILES: Cn1c(=O)oc2ccc(-c3ccc(C[C@@H](C#N)NS(=O)(=O)C4CCCCC4N)cc3)cc21] |
| 284A | 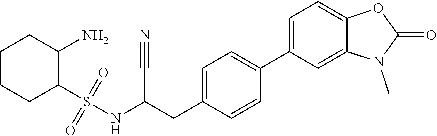 [SMILES: Cn1c(=O)oc2ccc(-c3ccc(CC(C#N)NS(=O)(=O)C4CCCCC4N)cc3)cc21] |
| 285 | 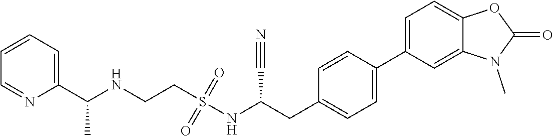 [SMILES: C[C@@H](NCCS(=O)(=O)N[C@H](C#N)Cc1ccc(-c2ccc3oc(=O)n(C)c3c2)cc1)c1ccccn1] |
| 285A | 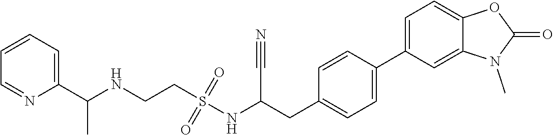 [SMILES: CC(NCCS(=O)(=O)NC(C#N)Cc1ccc(-c2ccc3oc(=O)n(C)c3c2)cc1)c1ccccn1] |
| 286 | 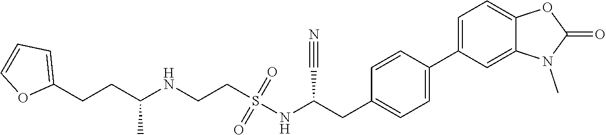 [SMILES: C[C@H](CCc1ccco1)NCCS(=O)(=O)N[C@H](C#N)Cc1ccc(-c2ccc3oc(=O)n(C)c3c2)cc1] |
| 286A | 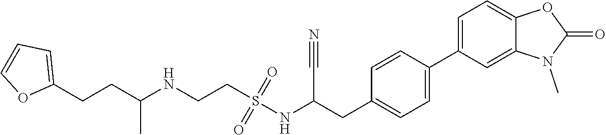 [SMILES: CC(CCc1ccco1)NCCS(=O)(=O)NC(C#N)Cc1ccc(-c2ccc3oc(=O)n(C)c3c2)cc1] |
| 287 | 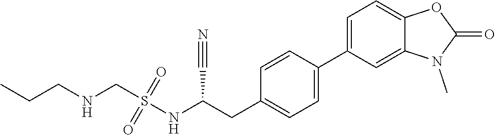 [SMILES: CCCNCS(=O)(=O)N[C@H](C#N)Cc1ccc(-c2ccc3oc(=O)n(C)c3c2)cc1] |
| 287A | 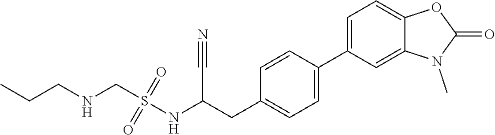 [SMILES: CCCNCS(=O)(=O)NC(C#N)Cc1ccc(-c2ccc3oc(=O)n(C)c3c2)cc1] |
| 288 | 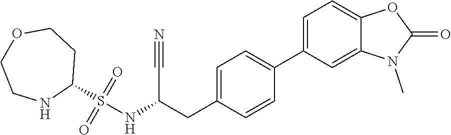 [SMILES: Cn1c(=O)oc2ccc(-c3ccc(C[C@@H](C#N)NS(=O)(=O)[C@H]4CCOCCN4)cc3)cc21] |
| 288A | 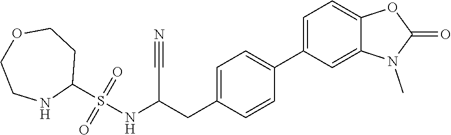 [SMILES: Cn1c(=O)oc2ccc(-c3ccc(CC(C#N)NS(=O)(=O)C4CCOCCN4)cc3)cc21] |
| 289 | 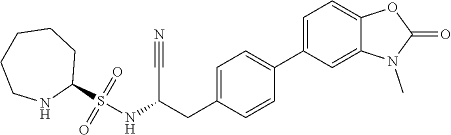 [SMILES: Cn1c(=O)oc2ccc(-c3ccc(C[C@@H](C#N)NS(=O)(=O)[C@@H]4CCCCCN4)cc3)cc21] |
| 289A | 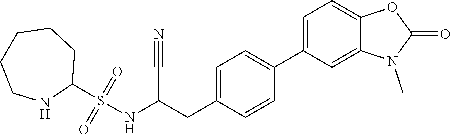 [SMILES: Cn1c(=O)oc2ccc(-c3ccc(CC(C#N)NS(=O)(=O)C4CCCCCN4)cc3)cc21] |
| 290 | 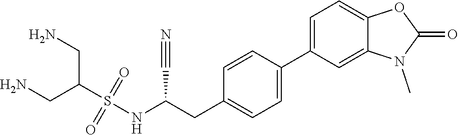 [SMILES: Cn1c(=O)oc2ccc(-c3ccc(C[C@@H](C#N)NS(=O)(=O)C(CN)CN)cc3)cc21] |
| 290A | 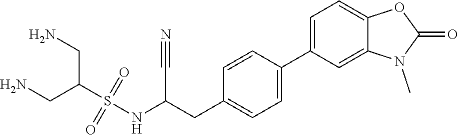 [SMILES: Cn1c(=O)oc2ccc(-c3ccc(CC(C#N)NS(=O)(=O)C(CN)CN)cc3)cc21] |
| 291 | 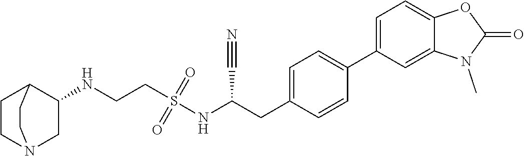 [SMILES: Cn1c(=O)oc2ccc(-c3ccc(C[C@@H](C#N)NS(=O)(=O)CCN[C@@H]4CN5CCC4CC5)cc3)cc21] |
| 291A | 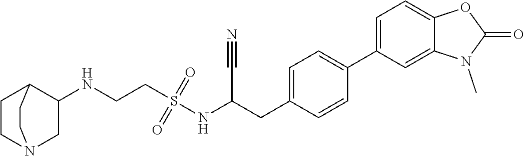 [SMILES: Cn1c(=O)oc2ccc(-c3ccc(CC(C#N)NS(=O)(=O)CCNC4CN5CCC4CC5)cc3)cc21] |
| 292 | 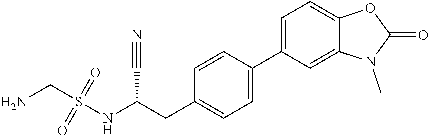 [SMILES: Cn1c(=O)oc2ccc(-c3ccc(C[C@@H](C#N)NS(=O)(=O)CN)cc3)cc21] |
| 292A | 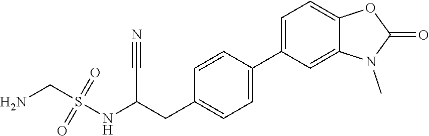 [SMILES: Cn1c(=O)oc2ccc(-c3ccc(CC(C#N)NS(=O)(=O)CN)cc3)cc21] |
| 280 | 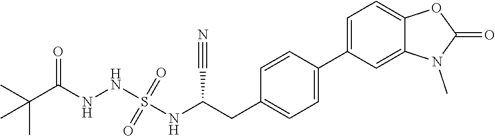 [SMILES: Cn1c(=O)oc2ccc(-c3ccc(C[C@@H](C#N)NS(=O)(=O)NNC(=O)C(C)(C)C)cc3)cc21] |
| 280A | 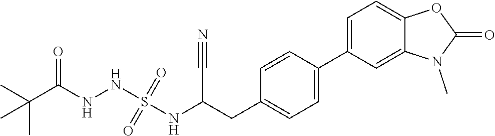 [SMILES: Cn1c(=O)oc2ccc(-c3ccc(CC(C#N)NS(=O)(=O)NNC(=O)C(C)(C)C)cc3)cc21] |
| 281 | 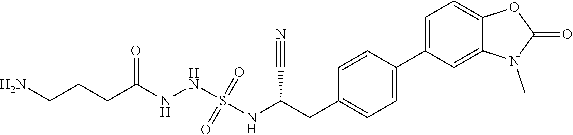 [SMILES: Cn1c(=O)oc2ccc(-c3ccc(C[C@@H](C#N)NS(=O)(=O)NNC(=O)CCCN)cc3)cc21] |
| 281A | 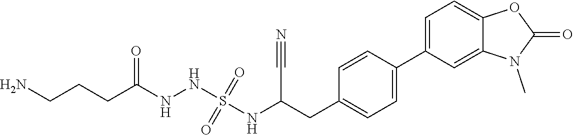 [SMILES: Cn1c(=O)oc2ccc(-c3ccc(CC(C#N)NS(=O)(=O)NNC(=O)CCCN)cc3)cc21] |
| 282 | 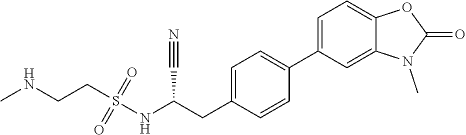 [SMILES: CNCCS(=O)(=O)N[C@H](C#N)Cc1ccc(-c2ccc3oc(=O)n(C)c3c2)cc1] |
| 282A | 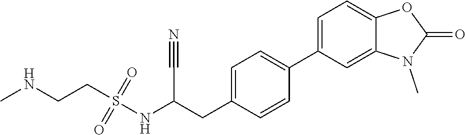 [SMILES: CNCCS(=O)(=O)NC(C#N)Cc1ccc(-c2ccc3oc(=O)n(C)c3c2)cc1] |
| 283 | 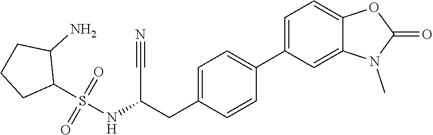 [SMILES: Cn1c(=O)oc2ccc(-c3ccc(C[C@@H](C#N)NS(=O)(=O)C4CCCC4N)cc3)cc21] |
| 283A | 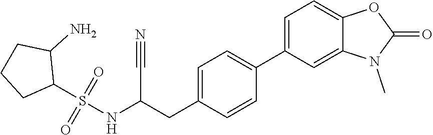 [SMILES: Cn1c(=O)oc2ccc(-c3ccc(CC(C#N)NS(=O)(=O)C4CCCC4N)cc3)cc21] |
| 284 | 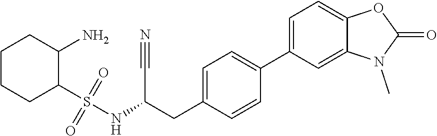 [SMILES: Cn1c(=O)oc2ccc(-c3ccc(C[C@@H](C#N)NS(=O)(=O)C4CCCCC4N)cc3)cc21] |
| 284A | 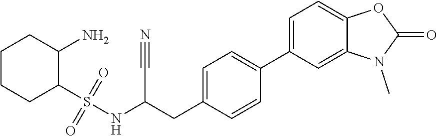 [SMILES: Cn1c(=O)oc2ccc(-c3ccc(CC(C#N)NS(=O)(=O)C4CCCCC4N)cc3)cc21] |
| 285 | 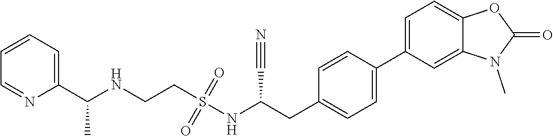 [SMILES: C[C@@H](NCCS(=O)(=O)N[C@H](C#N)Cc1ccc(-c2ccc3oc(=O)n(C)c3c2)cc1)c1ccccn1] |
| 285A | 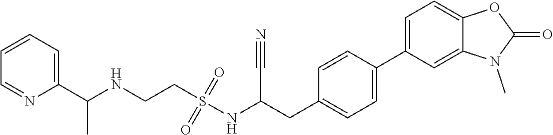 [SMILES: CC(NCCS(=O)(=O)NC(C#N)Cc1ccc(-c2ccc3oc(=O)n(C)c3c2)cc1)c1ccccn1] |
| 286 | 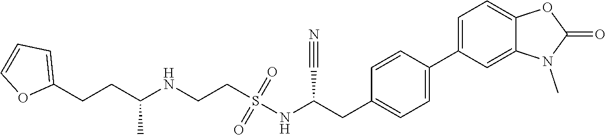 [SMILES: C[C@H](CCc1ccco1)NCCS(=O)(=O)N[C@H](C#N)Cc1ccc(-c2ccc3oc(=O)n(C)c3c2)cc1] |
| 286A | 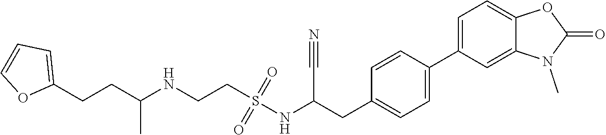 [SMILES: CC(CCc1ccco1)NCCS(=O)(=O)NC(C#N)Cc1ccc(-c2ccc3oc(=O)n(C)c3c2)cc1] |
| 287 | 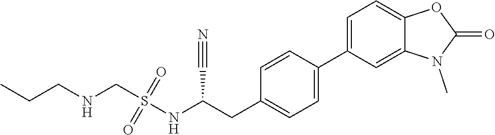 [SMILES: CCCNCS(=O)(=O)N[C@H](C#N)Cc1ccc(-c2ccc3oc(=O)n(C)c3c2)cc1] |
| 287A | 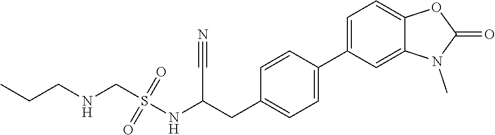 [SMILES: CCCNCS(=O)(=O)NC(C#N)Cc1ccc(-c2ccc3oc(=O)n(C)c3c2)cc1] |
| 288 | 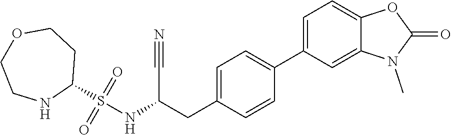 [SMILES: Cn1c(=O)oc2ccc(-c3ccc(C[C@@H](C#N)NS(=O)(=O)[C@H]4CCOCCN4)cc3)cc21] |
| 288A | 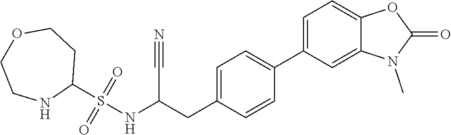 [SMILES: Cn1c(=O)oc2ccc(-c3ccc(CC(C#N)NS(=O)(=O)C4CCOCCN4)cc3)cc21] |
| 289 | 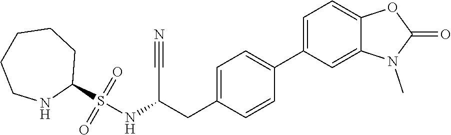 [SMILES: Cn1c(=O)oc2ccc(-c3ccc(C[C@@H](C#N)NS(=O)(=O)[C@@H]4CCCCCN4)cc3)cc21] |
| 289A | 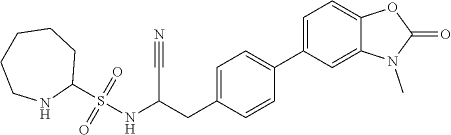 [SMILES: Cn1c(=O)oc2ccc(-c3ccc(CC(C#N)NS(=O)(=O)C4CCCCCN4)cc3)cc21] |
| 290 | 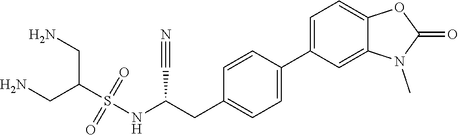 [SMILES: Cn1c(=O)oc2ccc(-c3ccc(C[C@@H](C#N)NS(=O)(=O)C(CN)CN)cc3)cc21] |
| 290A | 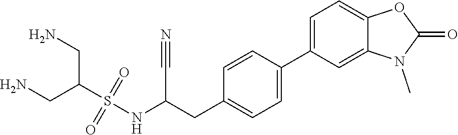 [SMILES: Cn1c(=O)oc2ccc(-c3ccc(CC(C#N)NS(=O)(=O)C(CN)CN)cc3)cc21] |
| 291 | 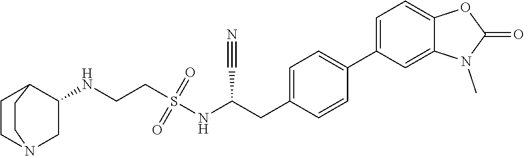 [SMILES: Cn1c(=O)oc2ccc(-c3ccc(C[C@@H](C#N)NS(=O)(=O)CCN[C@@H]4CN5CCC4CC5)cc3)cc21] |
| 291A | 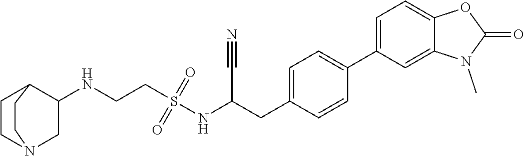 [SMILES: Cn1c(=O)oc2ccc(-c3ccc(CC(C#N)NS(=O)(=O)CCNC4CN5CCC4CC5)cc3)cc21] |
| 292 | 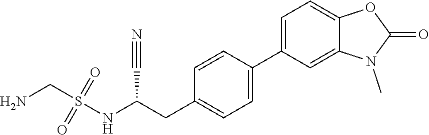 [SMILES: Cn1c(=O)oc2ccc(-c3ccc(C[C@@H](C#N)NS(=O)(=O)CN)cc3)cc21] |
| 292A | 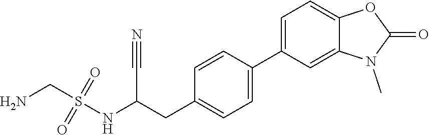 [SMILES: Cn1c(=O)oc2ccc(-c3ccc(CC(C#N)NS(=O)(=O)CN)cc3)cc21] |

In some embodiments, the present disclosure provides a compound having the following chemical formula:

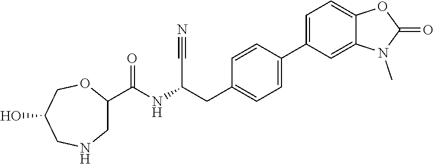 [SMILES: Cn1c(=O)oc2ccc(-c3ccc(C[C@@H](C#N)NC(=O)C4CNC[C@H](O)CO4)cc3)cc21]

or a pharmaceutically accept salt or deuterated form thereof.

In some embodiments, the present disclosure provides a compound having the following chemical formula:

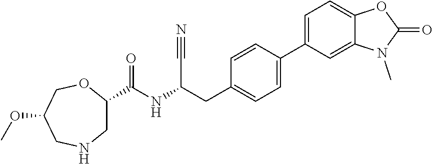 [SMILES: CO[C@H]1CNC[C@@H](C(=O)N[C@H](C#N)Cc2ccc(-c3ccc4oc(=O)n(C)c4c3)cc2)OC1]

or a pharmaceutically accept salt or deuterated form thereof.

In one embodiment, certain compounds of Formula (I), (II) and/or (III) may exist as racemates and racemic mixtures, single enantiomers, individual diastereomers and diastereomeric mixtures. It is to be understood that the present disclosure encompasses all such isomeric forms, even though the compounds of Formula (I), (II) and (III) in their preferred forms, have either S (one chiral center), or in the case of two chiral centers, S,S stereochemistry. For example, irrespective of R¹, the backbone of the compounds of Formula (I) and (II), in some embodiments, have two chiral centers. For the compounds of the invention, chiral center 1 is the substituted carbon atom to which a cyano group, —NH—, and a —CH₂— group are attached. In a preferred embodiment, the present disclosure encompasses the compounds of the invention with the (S)-configuration at chiral center 1. In another embodiment, however, compounds disclosed herein have the (R) configuration at chiral center 1. Chiral center 2, for certain compounds disclosed herein, for example, compounds of Formula (I) and/or (II), is the carbon atom of the R¹ group that attaches to the CO group. The present disclosure encompasses the compounds of the invention with the (S)-configuration at chiral center 2; and the (R)-configuration at chiral center 2, although the(S) configuration is preferred.

Where an acid or base co-former is a solid at room temperature and there is no or only partial proton transfer between the compound of Formula (I), (II), or (III), and such an acid or base co-former, a co-crystal of the co-former and compound of Formula (I), (II), or (III), may result rather than a salt. All such cocrystal forms of the compound of Formula (I), (II), or (III), are encompassed by the present disclosure. The compounds of Formula (I), (II), or (III), may form mixtures of its salt and co-crystal forms. It is also to be understood that the present disclosure encompasses salt/co-crystal mixtures of the compound of Formula (I), (II), or (III).

Salts and co-crystals may be characterized using well known techniques, for example X-ray powder diffraction, single crystal X-ray diffraction (for example to evaluate proton position, bond lengths or bond angles), solid state NMR, (to evaluate for example, C, N or P chemical shifts) or spectroscopic techniques (to measure for example, O—H, N—H or COOH signals and IR peak shifts resulting from hydrogen bonding).

It is also to be understood that certain compounds of Formula (I), (II), or (III), may exist in solvated form, e.g., hydrates, including solvates of a pharmaceutically acceptable salt of a compound of Formula (I), (II), or (III).

Certain compounds of Formula (I), (II), or (III), may also contain linkages (e.g., carbon-carbon bonds, carbon-nitrogen bonds such as amide bonds) wherein bond rotation is restricted about that particular linkage, e.g., restriction resulting from the presence of a ring bond or double bond. Accordingly, it is to be understood that the present disclosure encompasses all such isomers. Certain compound of Formula (I), (II), or (III), may also contain multiple tautomeric forms. It is to be understood that the present disclosure encompasses all such tautomeric forms. Stereoisomers may be separated using conventional techniques, e.g., chromatography or fractional crystallization, or the stereoisomers may be made by stereoselective synthesis.

In a further embodiment, the compounds of Formula (I), (II), or (III) encompass any isotopically-labeled (or “radio-labelled”) derivatives of a compound of Formula (I), (II), or (III). Such a derivative is a derivative of a compound of Formula (I), (II), or (III) wherein one or more atoms are replaced by an atom having an atomic mass or mass number different from the atomic mass or mass number typically found in nature. Examples of radionuclides that may be incorporated include 2H (also written as “D” for deuterium).

In a further embodiment, the compounds of Formula (I), (II), or (III) may be administered in the form of a prodrug which is broken down in the human or animal body to give a compound of the Formula (I), (II) or (III). Examples of prodrugs include in vivo hydrolysable esters of a compound of Formula (I), (II) or (III).

An in vivo hydrolysable (or cleavable) ester of a compound of the Formula (I), (II), or (III), that contains a carboxy or a hydroxy group is, for example, a pharmaceutically acceptable ester which is hydrolyzed in the human or animal body to produce the parent acid or alcohol. For examples of ester prodrugs derivatives, see, e.g., Curr. Drug. Metab. 2003, 4, 461. Various other forms of prodrugs are known in the art. See, e.g., Nature Reviews Drug Discovery 2008, 7,255 and references cited therein.

The compounds provided herein can be synthesized by a variety of synthetic routes and the final selection of the route will depend on the R¹, L, and R² substituents; specific routes may also require additional protecting group strategies. One such route includes an early-stage Burgess dehydration of an appropriate N-Boc protected “L” starting material followed by Suzuki coupling to prepare the “L-R²” intermediate; subsequent N-Boc deprotection and amide bond coupling to an appropriate R¹ moiety to yield the final compound.

Another route of synthesis starts with Suzuki coupling to prepare a formamide “L-R²” intermediate, followed by N-Boc deprotection and amide bond formation to prepare a formamide derivative that is subjected to a late-stage Burgess dehydration to yield compounds of the invention.

Synthesis routes disclosed in PCT Publication No. WO 2015/110826; WO 2022/042591; and Banerjee et al. (2021). Bioorganic & Medicinal Chemistry Letters 47, 128202, the disclosure of each of which is incorporated by reference in their entireties, can also be used herein.

Routes of synthesis for various compounds of the invention are also provided in the Example section herein.

The compounds of Formula (I), (II) and (III), and their pharmaceutically acceptable salts, are DPP1 inhibitors, and thus may be used in any disease area where DPP1 plays a role. As such, in one aspect of the invention, a method of treatment is provided. The method of treatment, in one embodiment, comprises, administering to a subject in need of, a composition comprising an effective amount of a compound of Formula (I), (II) or (III), or a pharmaceutically acceptable salt of Formula (I), (II) or (III). The composition is administered to the patient for an administration period.

As used herein, “treatment” or “treating,” or “ameliorating” are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); (3) relieving the condition (for example, by causing regression, or reducing the severity of the state, disorder or condition or at least one of its clinical or subclinical symptoms).

The term “effective amount” or “therapeutically effective amount” refers to the amount of an agent that is sufficient to achieve an outcome, for example, to effect beneficial or desired results. The therapeutically effective amount may vary depending upon one or more of: the subject and disease condition being treated, the weight and age of the subject, the severity of the disease condition, the manner of administration and the like.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older.

For example, in one embodiment, a compound of the present invention is administered to a patient in a method for treating an obstructive disease of the airway. The obstructive disease of the airway, in one embodiment, is asthma (e.g., bronchial, allergic, intrinsic, extrinsic, exercise-induced, drug-induced (including aspirin and NSAID-induced and dust-induced asthma, both intermittent and persistent and of all severities) airway hyper-responsiveness, chronic obstructive pulmonary disease (COPD), bronchitis (e.g., infectious bronchitis, eosinophilic bronchitis), emphysema, cystic fibrosis (CF), bronchiectasis (e.g., non-CF bronchiectasis (NCFBE) and bronchiectasis associated with CF), cystic fibrosis; sarcoidosis; alpha-1 antitrypsin (AlAT) deficiency, farmer's lung and related diseases, hypersensitivity pneumonitis, lung fibrosis (including idiopathic pulmonary fibrosis, cryptogenic fibrosing alveolitis, idiopathic interstitial pneumonias, fibrosis complicating anti-neoplastic therapy and chronic infection, including tuberculosis and aspergillosis and other fungal infections), complications of lung transplantation, vasculitic and thrombotic disorders of the lung vasculature, pulmonary hypertension (e.g., pulmonary arterial hypertension), pulmonary hypertension due to left heart disease, pulmonary hypertension associated with chronic lung diseases, antitussive activity including treatment of chronic cough associated with inflammatory and secretory conditions of the airways, iatrogenic cough, acute and chronic rhinitis including rhinitis medicamentosa, and vasomotor rhinitis; perennial and seasonal allergic rhinitis including rhinitis nervosa (hay fever), nasal polyposis; acute viral infection including the common cold, and infection due to a respiratory virus (e.g., respiratory syncytial virus, influenza, coronavirus (including SARS) and adenovirus), acute lung injury, acute respiratory distress syndrome (ARDS), as well as exacerbations of each of the foregoing respiratory tract disease states.

In one embodiment, a compound of the present invention is administered to a patient in a method of treating heart failure. The heart failure, in one embodiment, is heart failure with preserved ejection fraction or heart failure. In another embodiment, the heart failure is heart failure with reduced ejection fraction.

Cystic fibrosis (CF) is caused by abnormalities in the CF transmembrane conductance regulator protein, causing chronic lung infections (particularly with Pseudomonas aeruginosa) and excessive inflammation, and leading to bronchiectasis, declining lung function, respiratory insufficiency and quality of life. The inflammatory process is dominated by neutrophils that produce NE, as well as other destructive NSPs including CatG and PR3, that directly act upon extracellular matrix proteins and play a role in the host response to inflammation and infection (Dittrich et al., Eur Respir J. 2018; 51(3)). The methods provided herein employ reversible inhibitors of DPP1. Without wishing to be bound by theory, it is thought that the compounds of Formula (I), (II), or (III), administered via the methods provided herein have beneficial effects via inhibiting the activation of NSPs and decreasing inflammation, which in turn leads to a decrease in pulmonary exacerbations, a decrease in the rate of pulmonary exacerbations, and/or an improvement in lung function (e.g., forced expiratory volume in 1 second [FEV₁]) in CF patients.

In one embodiment, a method is provided for treating CF comprising administering to a CF patient in need of treatment, a composition comprising an effective amount of a compound of Formula (I), (II), or (III), or a pharmaceutically acceptable salt thereof. Administration routes include oral administration. Administration schedules and administration periods can be determined by the user of the method, e.g., a prescribing physician. In one embodiment, administration is once daily. In another embodiment, administration is twice daily. In another embodiment, administration is every other day, every third day, 3× per week or 4× per week.

In one CF treatment method, a composition comprising an effective amount of a compound of Formula (I), (II), or (III), or a pharmaceutically acceptable salt thereof, is administered to a CF patient in need of treatment for an administration period. The method comprises improving the lung function of the patient during the administration period, as compared to the lung function of the patient prior to the administration period. In a further embodiment, the compound is administered orally, once daily. The improvement in lung function in one embodiment, is measured by spirometry.

Improving the lung function of the patient, in one embodiment, comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75%)}), as compared to the respective value prior to the administration period. Increasing, in one embodiment, is by about 5%, by about 10%, by about 15%, by about 20%, by about 25%, by about 30%, by about 35%, by about 40%, by about 45% or by about 50% of the respective value. Increasing, in one embodiment, is by at least about 5%, by at least about 10%, by at least about 15%, by at least about 20%, by at least about 25%, by at least about 30%, by at least about 35%, by at least about 40%, by at least about 45% or by at least about 50%. In yet another embodiment, the increasing is by about 5% to about 50%, by about 5% to about 40%, by about 5% to about 30% or by about 5% to about 20%. In even another embodiment, increasing is by about 10% to about 50%, by about 15% to about 50%, by about 20% to about 50%, or by about 25% to about 50%.

In one embodiment of a method provided herein, a composition comprising an effective amount of a compound of Formula (I), (II), or (III), or a pharmaceutically acceptable salt thereof, is administered to a bronchiectasis patient in need of treatment for an administration period. Bronchiectasis is considered a pathological endpoint that results from many disease processes and is a persistent or progressive condition characterized by dilated thick-walled bronchi. The symptoms vary from intermittent episodes of expectoration and infection localized to the region of the lung that is affected to persistent daily expectoration often of large volumes of purulent sputum. Bronchiectasis may be associated with other non-specific respiratory symptoms. The underlying pathological process of bronchiectasis, without wishing to be bound by theory, has been reported as damage to the airways which results from an event or series of events where inflammation is central to the process (Guideline for non-CF Bronchiectasis, Thorax, July 2010, V. 65 (Suppl 1), incorporated by reference herein in its entirety for all purposes).

Bronchiectasis is considered a pathological endpoint that results from many disease processes and is a persistent or progressive condition characterized by dilated thick-walled bronchi. The symptoms vary from intermittent episodes of expectoration and infection localized to the region of the lung that is affected to persistent daily expectoration often of large volumes of purulent sputum. Bronchiectasis may be associated with other non-specific respiratory symptoms. The underlying pathological process of bronchiectasis, without wishing to be bound by theory, has been reported as damage to the airways which results from an event or series of events where inflammation is central to the process (Guideline for non-CF Bronchiectasis, Thorax, July 2010, V. 65 (Suppl 1), incorporated by reference herein in its entirety for all purposes).

The methods provided herein employ reversible inhibitors of DPP1. Without wishing to be bound by theory, it is thought that the compounds of Formula (I), (II), or (III), administered via the methods provided herein have beneficial effects via decreasing inflammation and mucus hypersecretion, which in some embodiments, leads to a decrease in pulmonary exacerbations, a decrease in the rate of pulmonary exacerbations, and/or an improvement in lung function (cough, sputum production, and forced expiratory volume in 1 second [FEV₁]) in bronchiectasis patients. Without wishing to be bound by theory, it is thought that the methods provided herein modify bronchiectasis progression by reducing the accelerated rate of lung function decline or lung tissue destruction.

In one embodiment, the bronchiectasis is non-CF bronchiectasis.

In one embodiment, the method for treating bronchiectasis comprises improving lung function of the patient during the administration period, as compared to the lung function of the patient prior to the administration period.

A pulmonary exacerbation, in one embodiment, is characterized by three or more of the following symptoms exhibited for at least 48 hours by the patient: (1) increased cough; (2) increased sputum volume or change in sputum consistency; (3) increased sputum purulence; (4) increased breathlessness and/or decreased exercise tolerance; (5) fatigue and/or malaise; (6) hemoptysis. In a further embodiment, the three or more symptoms result in a physician's decision to prescribe an antibiotic(s) to the patient exhibiting the symptoms.

In one embodiment of a method for treating bronchiectasis, the method comprises decreasing the rate of pulmonary exacerbation in the subject, compared to the rate of pulmonary exacerbation experienced by the subject prior to the administration period of the composition, or compared to a control subject with bronchiectasis that is not subject to the method of treatment. In a further embodiment, the bronchiectasis is non-CF bronchiectasis.

In another aspect, a method for treating chronic rhinosinusitis (CRS) in a subject in need thereof is provided. The method comprises in one embodiment, administering to the subject for an administration period, a pharmaceutical composition comprising an effective amount of a compound of Formula (I), (II), or (III), or a pharmaceutically acceptable salt thereof.

The chronic rhinosinusitis is chronic rhinosinusitis without nasal polyps (CRSsNP), or chronic rhinosinusitis with nasal polyps (CRSwNP). In some embodiments, the chronic rhinosinusitis is chronic rhinosinusitis without nasal polyps (CRSsNP). In some embodiments, the chronic rhinosinusitis is chronic rhinosinusitis with nasal polyps (CRSwNP). In some embodiments, the chronic rhinosinusitis is refractory chronic rhinosinusitis. In some embodiments, the refractory chronic rhinosinusitis is refractory chronic rhinosinusitis without nasal polyps (CRSsNP). In some embodiments, the refractory chronic rhinosinusitis is refractory chronic rhinosinusitis with nasal polyps (CRSwNP).

In some embodiments, the subject exhibits one or more symptoms of CRS. In some embodiments, the one or more symptoms of CRS are: (a) nasal congestion; (b) nasal obstruction; (c) nasal discharge; (d) post-nasal drip; (e) facial pressure; (f) facial pain; (g) facial fullness; (h) reduced smell; (i) depression; (j) mucosal edema; (k) mucopurulent discharge; (1) obstruction of the middle meatus; (m) mucosal changes within the ostiomeatal complex and sinuses; (n) rhinorrhea; or (o) any combinations thereof. In some embodiments, obstruction of the middle meatus is mucosal obstruction, edematous obstruction, or a combination thereof.

In some embodiments, the administration of the pharmaceutical composition reduces, diminishes the severity of, delays the onset of, or eliminates one or more symptoms of CRS. In some embodiments, the one or more symptoms of CRS are: (a) nasal congestion; (b) nasal obstruction; (c) nasal discharge; (d) post-nasal drip; (e) facial pressure; (f) facial pain; (g) facial fullness; (h) reduced smell; (i) depression; (j) mucosal edema; (k) mucopurulent discharge; (l) obstruction of the middle meatus; (m) mucosal changes within the ostiomeatal complex and sinuses; (n) rhinorrhea; (o) or any combinations thereof. In some embodiments, the administration of the pharmaceutical composition enhances sinus drainage.

In some embodiments, the methods comprise reducing a composite severity score of one or more symptoms of CRS. As used herein, the “composite severity score” is a quantitative measure of all the symptoms of CRS exhibited by the subject. In some embodiments, the composite severity score is a sum total of all the daily symptoms exhibited by the subject. In some embodiments, the composite severity score is reduced during or subsequent to the administration period, as compared to the composite severity score measured prior to the administration period. In some embodiments, the one or more symptoms of CRS exhibited by the subject may be any symptoms described herein or known in the art to be associated with CRS. In some embodiments, the one or more symptoms of CRS are: nasal congestion, reduced smell, rhinorrhea, or any combination thereof. In some embodiments, the rhinorrhea is anterior rhinorrhea. In some embodiments, the rhinorrhea is posterior rhinorrhea.

In some embodiments, the methods comprise decreasing the Sino-Nasal Outcome Test-22 (SNOT-22) score of the subject during the administration period or subsequent to the administration period, compared to the SNOT-22 score of the subject prior to the administration period. As used herein, “SNOT-22” is a patient-reported measure of outcome developed for use in CRS with or without nasal polyps and contains 22 individual questions. The questions cover a broad range of health and health-related quality of life problems including physical problems, functional limitations and emotional consequences. The theoretical range of the SNOT-22 score is 0-110, with lower scores implying a better health-related quality of life. Further details of SNOT-22 are provided in Hopkins, et al., Clin. Otolaryngol. 2009, 34, 447-454, and Kennedy, et al., Ann Allergy Asthma Immunol. 2013 October; 111(4): 246-251, the contents of which are incorporated herein by reference in its entirety.

Hidradenitis suppurativa (HS) is a chronic relapsing inflammatory disorder. The symptoms include skin lesions that are often associated hair follicles, and may be painful, inflamed and/or swollen. In some cases, when the skin lesions heal, they can recur, and may lead to tunnels under the skin and progressive scarring. Since HS is a chronic condition, it can persist for many years and also, worsen over time, with serious effects on quality of life, psychological and emotional well-being. In fact, HS pateints have increased rates of anxiety and depression with a risk of suicide two and a half times that of the general population.

HS patients are categorized according to disease severity, termed Hurley staging, as mild (Stage I), moderate (Stage II), or severe (Stage III). Although more than 200,000 cases of HS are diagnosed in the U.S. per year, this disease can be difficult to diagnose and requires specialized care. HS may be mistaken for an infection, an ingrown hair or other conditions. Moreover, current treatment options are limited and lack efficacy.

In one aspect, a method of treating HS in a subject in need thereof is provided. The method comprises in one embodiment, administering to the subject for an administration period, a pharmaceutical composition comprising an effective amount of a compound of Formula (I), (II), or (III), or a pharmaceutically acceptable salt thereof. In a further embodiment, the method of treating HS comprises reducing neutrophilic inflammation in the subject.

The HS in one embodiment, is Hurley Stage I HS, Hurley Stage II HS or Hurley Stage III HS. In some embodiments, the HS is Hurley Stage I HS. In some embodiments, the HS is Hurley Stage II HS. In some embodiments, the HS is Hurley Stage III HS.

The disclosure provides methods of treating cancer in a subject in need thereof, comprising, administering to the subject, a pharmaceutical composition comprising an effective amount of any one of the compounds disclosed herein. The disclosure provides methods of treating cancer-induced pain in a subject having cancer, comprising, administering to the subject for an administration period, a pharmaceutical composition comprising an effective amount of any one of the compounds disclosed herein. In some embodiments, the cancer-induced pain is cancer-induced bone pain. The disclosure also provides methods of treating cancer-induced bone pain in a subject having cancer, comprising, administering to the subject for an administration period, a pharmaceutical composition comprising an effective amount of any one of the compounds disclosed herein.

In some embodiments, the cancer comprises a primary solid tumor. In some embodiments, the cancer is selected from the group consisting of bladder cancer, lung cancer, brain cancer, ovarian cancer, pancreatic cancer, colorectal cancer, prostate cancer, liver cancer, hepatocellular carcinoma, kidney cancer, stomach cancer, skin cancer, fibroid cancer, lymphoma, virus-induced cancer, oropharyngeal cancer, testicular cancer, thymus cancer, thyroid cancer, melanoma, and bone cancer.

In some embodiments, the cancer is bladder cancer. In some embodiments, the cancer is lung cancer. In some embodiments, the cancer is brain cancer. In some embodiments, the cancer is ovarian cancer. In some embodiments, the cancer is pancreatic cancer. In some embodiments, the cancer is colorectal cancer. In some embodiments, the cancer is prostate cancer. In some embodiments, the cancer is liver cancer. In some embodiments, the cancer is hepatocellular carcinoma. In some embodiments, the cancer is kidney cancer. In some embodiments, the cancer is stomach cancer. In some embodiments, the cancer is skin cancer. In some embodiments, the cancer is fibroid cancer. In some embodiments, the cancer is lymphoma. In some embodiments, the cancer is virus-induced cancer. In some embodiments, the cancer is oropharyngeal cancer. In some embodiments, the cancer is testicular cancer. In some embodiments, the cancer is thymus cancer. In some embodiments, the cancer is thyroid cancer. In some embodiments, the cancer is melanoma. In some embodiments, the cancer is bone cancer. In some embodiments, the fibroid cancer is leiomyosarcoma.

In some embodiments, the breast cancer comprises ductal carcinoma, lobular carcinoma, medullary carcinoma, colloid carcinoma, tubular carcinoma, or inflammatory breast cancer. In some embodiments, the breast cancer comprises ductal carcinoma. In some embodiments, the breast cancer comprises lobular carcinoma. In some embodiments, the breast cancer comprises medullary carcinoma. In some embodiments, the breast cancer comprises colloid carcinoma. In some embodiments, the breast cancer comprises tubular carcinoma. In some embodiments, the breast cancer comprises inflammatory breast cancer.

In some embodiments, the breast cancer is triple-negative breast cancer. In some embodiments, the breast cancer does not respond to hormonal therapy or therapeutics that target the HER2 protein receptors.

In some embodiments, the lymphoma is Hodgkin's lymphoma, non-Hodgkin's lymphoma, diffuse large B-cell lymphoma, B-cell immunoblastic lymphoma, Natural Killer cell lymphoma, T-cell lymphoma, Burkitt lymphoma or Kaposi's Sarcoma. In some embodiments, the lymphoma is Hodgkin's lymphoma. In some embodiments, the lymphoma is non-Hodgkin's lymphoma. In some embodiments, the lymphoma is diffuse large B-cell lymphoma. In some embodiments, the lymphoma is B-cell immunoblastic lymphoma. In some embodiments, the lymphoma is Natural Killer cell lymphoma. In some embodiments, the lymphoma is T-cell lymphoma. In some embodiments, the lymphoma is Burkitt lymphoma. In some embodiments, the lymphoma is Kaposi's Sarcoma.

In some embodiments, the brain cancer is astrocytoma, anaplastic astrocytoma, glioblastoma multiforme, oligodendroglioma, ependymoma, meningioma, schwannoma, or medulloblastoma. In some embodiments, the brain cancer is astrocytoma. In some embodiments, the brain cancer is anaplastic astrocytoma. In some embodiments, the brain cancer is glioblastoma multiforme. In some embodiments, the brain cancer is oligodendroglioma. In some embodiments, the brain cancer is ependymoma. In some embodiments, the brain cancer is meningioma. In some embodiments, the brain cancer is schwannoma. In some embodiments, the brain cancer is medulloblastoma.

In some embodiments, the cancer is liquid tumor. In some embodiments, the liquid tumor is selected from the group consisting of acute myeloid leukemia (AML), acute lymphoblastic leukemia, acute lymphocytic leukemia, acute promyelocytic leukemia, chronic myeloid leukemia, hairy cell leukemia, myeloproliferative disorders, Natural Killer cell leukemia, blastic plasmacytoid dendritic cell neoplasm, chronic myelogenous leukemia (CML), mastocytosis, chronic lymphocytic leukemia (CLL), multiple myeloma (MM), and myelodysplastic syndrome (MDS). In some embodiments, the liquid tumor is acute myeloid leukemia (AML). In some embodiments, the liquid tumor is acute lymphoblastic leukemia. In some embodiments, the liquid tumor is acute lymphocytic leukemia. In some embodiments, the liquid tumor is acute promyelocytic leukemia. In some embodiments, the liquid tumor is chronic myeloid leukemia. In some embodiments, the liquid tumor is hairy cell leukemia. In some embodiments, the liquid tumor is a myeloproliferative disorder. In some embodiments, the liquid tumor is Natural Killer cell leukemia. In some embodiments, the liquid tumor is blastic plasmacytoid dendritic cell neoplasm. In some embodiments, the liquid tumor is chronic myelogenous leukemia (CML). In some embodiments, the liquid tumor is mastocytosis. In some embodiments, the liquid tumor is chronic lymphocytic leukemia (CLL). In some embodiments, the liquid tumor is multiple myeloma (MM). In some embodiments, the liquid tumor is myelodysplastic syndrome (MDS).

In some embodiments, the cancer is a pediatric cancer. In some embodiments, the pediatric cancer is neuroblastoma, Wilms tumor, rhabdomyosarcoma, retinoblastoma, osteosarcoma or Ewing sarcoma. In some embodiments, the pediatric cancer is neuroblastoma. In some embodiments, the pediatric cancer is Wilms tumor. In some embodiments, the pediatric cancer is rhabdomyosarcoma. In some embodiments, the pediatric cancer is retinoblastoma. In some embodiments, the pediatric cancer is osteosarcoma. In some embodiments, the pediatric cancer is Ewing sarcoma.

In some embodiments, the cancer is metastatic cancer. In some embodiments, the subject is at a risk for developing metastatic cancer. In some embodiments, the metastatic cancer comprises metastasis of breast cancer to the brain, bone, pancreas, lymph nodes, and/or liver. In some embodiments, the metastatic cancer comprises metastasis of bone cancer to the lung. In some embodiments, the metastatic cancer comprises metastasis of colorectal cancer to the peritoneum, the pancreas, the stomach, the lung, the liver, the kidney, and/or the spleen. In some embodiments, the metastatic cancer comprises metastasis of stomach cancer to the mesentery, the spleen, the pancreas, the lung, the liver, the adrenal gland, and/or the ovary. In some embodiments, the metastatic cancer comprises metastasis of leukemia to the lymph nodes, the lung, the liver, the hind limb, the brain, the kidney, and/or the spleen. In some embodiments, the metastatic cancer comprises metastasis of liver cancer to the intestine, the spleen, the pancreas, the stomach, the lung, and/or the kidney. In some embodiments, the metastatic cancer comprises metastasis of lymphoma to the kidney, the ovary, the liver, the bladder, and/or the spleen.

In some embodiments, the metastatic cancer comprises metastasis of hematopoietic cancer to the intestine, the lung, the liver, the spleen, the kidney, and/or the stomach. In some embodiments, the metastatic cancer comprises metastasis of melanoma to lymph nodes and/or the lung. In some embodiments, the metastatic cancer comprises metastasis of pancreatic cancer to the mesentery, the ovary, the kidney, the spleen, the lymph nodes, the stomach, and/or the liver. In some embodiments, the metastatic cancer comprises metastasis of prostate cancer to the lung, the pancreas, the kidney, the spleen, the intestine, the liver, the bone, and/or the lymph nodes. In some embodiments, the metastatic cancer comprises metastasis of ovarian cancer to the diaphragm, the liver, the intestine, the stomach, the lung, the pancreas, the spleen, the kidney, the lymph nodes, and/or the uterus. In some embodiments, the metastatic cancer comprises metastasis of myeloma to the bone.

In some embodiments, the metastatic cancer comprises metastasis of lung cancer to the bone, the brain, the lymph nodes, the liver, the ovary, and/or the intestine. In some embodiments, the metastatic cancer comprises metastasis of kidney cancer to the liver, the lung, the pancreas, the stomach, the brain, and/or the spleen. In some embodiments, the metastatic cancer comprises metastasis of bladder cancer to the bone, the liver and/or the lung. In some embodiments, the metastatic cancer comprises metastasis of thyroid cancer to the bone, the liver and/or the lung.

In some embodiments, the methods disclosed herein comprise treating cancer-induced bone pain (CIBP) in a subject having metastasis of a cancer to the bone. In some embodiments, the subject has metastasis of prostate cancer, breast cancer, lung cancer, or myeloma to the bone. In some embodiments, the subject is identified as having metastasis to the bone by the use of any one of the following methods: plain film radiography, computed tomography, technetium 99m bone scan, magnetic resonance imaging, fluorodeoxyglucose positron emission tomography, fluorine positron emission tomography, and/or choline positron emission tomography, but is not yet feeling cancer-induced bone pain. In some embodiments, the subject is suffering from cancer-induced bone pain, which is indicative of metastasis of a previously treated or untreated primary tumor to the bone. In some embodiments, the cancer has metastasized to vertebrae, pelvis, long bones, or ribs.

In some embodiments, administration of the composition diminishes the severity of, delays the onset of, or eliminates a symptom of cancer. In some embodiments, the symptom of cancer is cancer-induced bone pain (CIBP). In some embodiments, the CIBP is neuropathic pain. In some embodiments, the CIBP is inflammatory pain. In some embodiments, the CIBP is spontaneous pain. In some embodiments, the symptom of cancer is nociceptive hypersensitivity. In some embodiments, the symptom of cancer is allodynia. In some embodiments, the allodynia is tactile allodynia. In some embodiments, the tactile allodynia is static mechanical allodynia. In some embodiments, the tactile allodynia is dynamic mechanical allodynia. In some embodiments, the subject has bone cancer or metastasis to the bone.

In yet another embodiment of the present invention, a method for treating lupus nephritis (LN) in a subject in need thereof is provided. The method comprises administering to the subject for an administration period, a pharmaceutical composition comprising an effective amount of a compound of Formula (I), (II), or (III), or a pharmaceutically acceptable salt thereof.

Rheumatoid arthritis (RA) is characterized by inflammation and thickening of the joint capsule, together with an effect on the underlying bone and cartilage. Currently, the cause of RA is unknown and no satisfactory cure for RA is available. While a number of therapeutic agents have been developed and utilized to alleviate pain and inflammation associated with the disease, such as disease-modifying antirheumatic drugs (DMARDs) and non-steroidal anti-inflammatory agents (NSAIDs), they often produce intolerable side effects. To addresses this and other needs, the present invention, in one embodiment, provides a method for treating RA using reversible inhibitors of DPP1 of Formula (I), (II), or (III). In one embodiment, a method of for treating RA in a subject in need thereof is provided, and comprises administering to the subject for an administration period, a pharmaceutical composition comprising an effective amount of a compound of Formula (I), (II), or (III), or a pharmaceutically acceptable salt thereof. In a further embodiment, the method comprises reducing neutrophilic inflammation in the subject.

Inflammatory bowel disease (IBD) is a group of inflammatory conditions that affect the colon and small intestine. The most common IBDs are Crohn's disease and ulcerative colitis. The present invention, in one embodiment, addresses the need for novel IBD therapies. Specifically, in one embodiment, a method for treating an inflammatory bowel disease (IBD) in a subject in need thereof is provided. The method comprises administering to the subject for an administration period, a pharmaceutical composition comprising an effective amount of a compound of Formula (I), (II), or (III), or a pharmaceutically acceptable salt thereof.

In a further embodiment, the IBD is Crohn's disease or ulcerative colitis. In even a further embodiment, the method comprises reducing neutrophilic inflammation in the subject.

The length of the administration period in any given case may depend on the nature and severity of the condition being treated and/or prevented and be determined by the physician. In one embodiment, the administration period starts at about the time of condition/disease diagnosis and continues for the lifetime of the patient.

In some embodiments, the administration period is about 30 days, about 35 days, about 40 days, about 45 days, about 50 days, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 30 months, about 36 months, about 4 years, about 5 years, about 10 years, about 15 years or about 20 years. In some embodiments, the compounds or compositions disclosed herein may be administered for a period of about 24 weeks. In some embodiments, the compounds or compositions disclosed herein may be administered for a period of about 52 weeks. In yet another embodiment, the administration period is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, at least about 30 months, at least about 36 months, at least about 4 years, at least about 5 years, at least about 10 years, at least about 15 years or at least about 20 years.

In some embodiments, the administration period for the methods provided herein is at least about 30 days, at least about 35 days, at least about 40 days, at least about 45 days, at least about 50 days, at least about 2 months, at least about 3 months, at least about 4 months or at least about 6 months, at least about 7 months, at least about 8 months, at least about 9 months, at least about 10 months, at least about 11 months, at least about 1 year, at least about 2 years, at least about 3 years, at least about 4 years, at least about 5 years. The administration period for the methods provided herein, in another embodiment, is from about 30 days to about 180 days. In another embodiment, the administration period is from about 30 days to about 36 months, or from about 30 days to about 30 months, or from about 30 days to about 24 months, or from about 30 days to about 18 months, or from about 30 days to about 12 months, or from about 30 days to about 6 months, or from about 6 months to about 30 months, or from about 6 months to about 24 months, or from about 6 months to about 18 months, or from about 12 months to about 36 months, or from about 12 months to about 24 months.

In one embodiment, the administration period is from about 1 year to about 30 years. For example, the administration period, in one embodiment, is from about 1 year to about 25 years, 1 year to about 20 years, from about 1 year to about 15 years, from about 1 year to about 10 years, from about 1 year to about 5 years, from about 1 year to about 3 years, from about 1 year to about 2 years, from about 2 years to about 15 years, from about 2 year to about 10 years, from about 2 years to about 8 years, from about 2 year to about 5 years, from about 2 years to about 4 years, or from about 2 years to about 3 years.

In one embodiment of the method, the subject is administered the composition once daily during the administration period. In another embodiment, the patient is administered the composition twice daily, or every other day, or once a week during the administration period. In another embodiment, administration is every other day, every third day, 3× per week or 4× per week during the administration period.

In one embodiment, the oral dosage form is administered once daily during the administration period. In a further embodiment, the oral dosage form is administered at approximately the same time every day, e.g., prior to breakfast. In another embodiment, the composition comprising an effective amount of Formula (I), (II) or (III) is administered 2× daily during the administration period. In yet another embodiment, the composition comprising an effective amount of Formula (I), (II) or (III) is administered 1× per week, every other day, every third day, 2× per week, 3× per week, 4× per week, or 5× per week during the administration period.

Administration, in one embodiment, is via the oral route. In a further embodiment, the composition is administered once daily.

The dosage administered will vary with the compound of Formula (I), (II) or (III), employed, the mode of administration, and the treatment outcome desired. For example, in one embodiment, the daily dosage of the compound of Formula (I), (II) or (III), if inhaled, may be in the range from 0.05 micrograms per kilogram body weight (μg/kg) to 100 micrograms per kilogram body weight (μg/kg). Alternatively, in one embodiment, if the compound of Formula (I), (II) or (III), is administered orally, then the daily dosage of the compound of the disclosure may be in the range from 0.01 micrograms per kilogram body weight (μg/kg) to 100 milligrams per kilogram body weight (mg/kg).

The compounds of Formula (I), (II) or (III), or pharmaceutically acceptable salts thereof, may be used on their own but will generally be administered in the form of a pharmaceutical composition in which the Formula (I), (II) or (III), compound/salt (active ingredient) is in association with pharmaceutically acceptable adjuvant(s), diluents(s) or carrier(s). Conventional procedures for the selection and preparation of suitable pharmaceutical formulations are described in, for example, “Pharmaceuticals—The Science of Dosage Form Designs”, M. E. Aulton, Churchill Livingstone, 2nd Ed. 2002.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present disclosure also provides pharmaceutical composition(s) comprising a compound of Formula (I), (II) or (III), or a pharmaceutically acceptable salt thereof, as hereinbefore defined in association with pharmaceutically acceptable adjuvant(s), diluent(s) or carrier(s).

The disclosure further provides a process for the preparation of a pharmaceutical composition of the disclosure which comprises mixing a compound of Formula (I), (II) or (III), or a pharmaceutically acceptable salt thereof, as hereinbefore defined with a pharmaceutically acceptable adjuvant(s), diluents(s) or carrier(s).

The pharmaceutical compositions may be administered topically (e.g., to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g., by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

For oral administration the compound of the disclosure may be admixed with adjuvant(s), diluent(s) or carrier(s), for example, lactose, saccharose, sorbitol, mannitol; starch, for example, potato starch, corn starch or amylopectin; cellulose derivative; binder, for example, gelatine or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatine, talcum and titanium dioxide.

For the preparation of soft gelatine capsules, the compound of the disclosure may be admixed with, for example, a vegetable oil or polyethylene glycol. Hard gelatine capsules may contain granules of the compound using pharmaceutical excipients like the abovementioned excipients for tablets. Additionally, liquid or semisolid formulations of the compound of the disclosure may be filled into hard gelatine capsules.

Liquid preparations for oral application may be in the form of syrups, solutions or suspensions. Solutions, for example may contain the compound of the disclosure, the balance being sugar and a mixture of ethanol, water, glycerol and propylene glycol. Optionally such liquid preparations may contain coloring agents, flavoring agents, saccharine and/or carboxymethylcellulose as a thickening agent. Furthermore, other excipients known to those skilled in art may be used when making formulations for oral use.

### NUMBERED EMBODIMENTS OF THE DISCLOSURE

### Numbered Embodiments—Set A

In addition to the disclosure above, the Examples below, and the appended claims, the disclosure sets forth the following numbered embodiments.
1. A compound of formula (I):

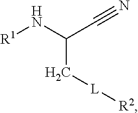

or a pharmaceutically acceptable salt thereof, wherein,
R¹ is

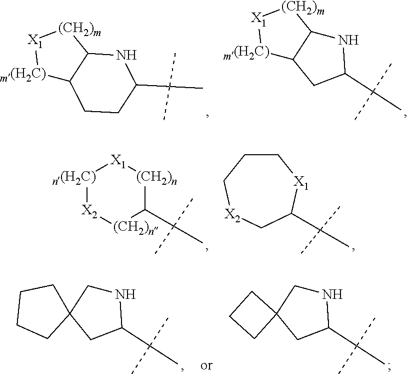

R² is

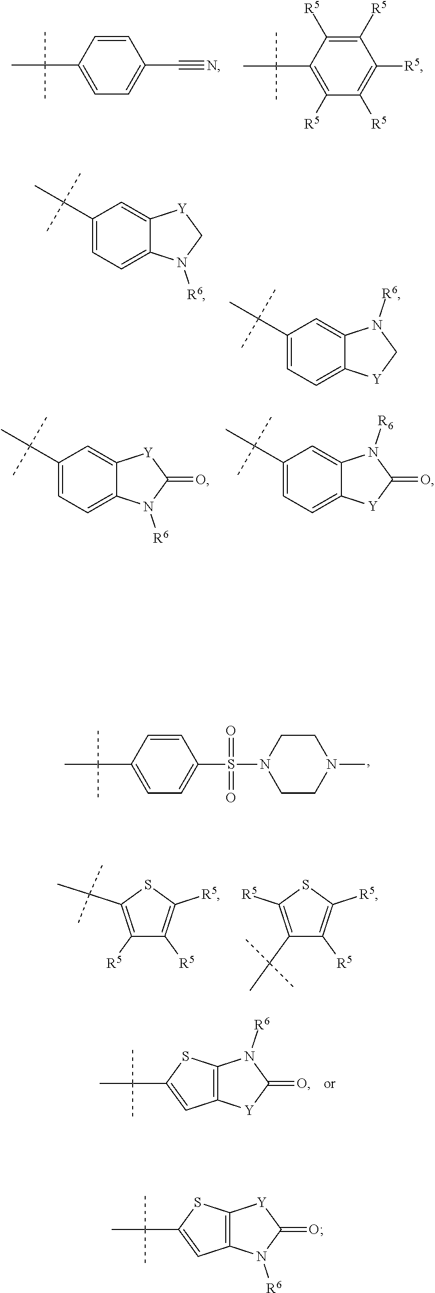

X₁, X₂ and X₃ are independently O, S, NR³ or CR³R⁴;
each R³, R⁴ and R⁵ is independently H, F, Cl, Br, I or C₁-C₆ alkyl;
each R⁶ is independently H or C₁-C₆ alkyl;
each Y is independently O, S, CHR⁶ or NR⁶;
m and m′ are each independently an integer from 0-3, and the total sum of m and m′ is ≤3;
each n, n′ and n″ is independently an integer from 0-3, and the total sum of n, n′ and n″ is ≤4; and
L is

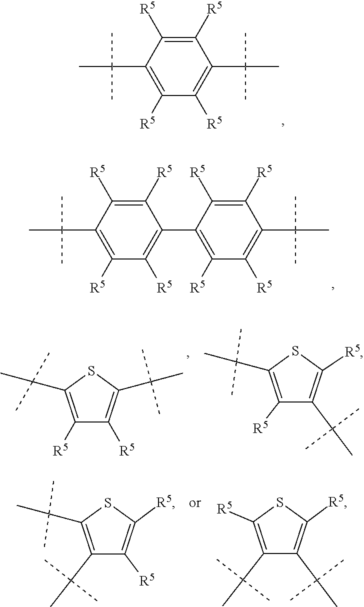

provided that,
   (i) when R¹ is

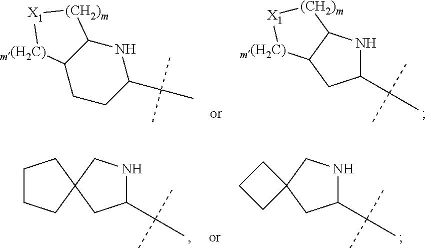

and X₁ is —CH₂—, then L and R² taken together, is not

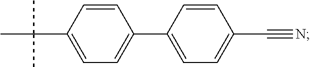

or
(ii) when R¹ is

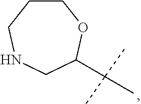

then L and R² taken together, is not

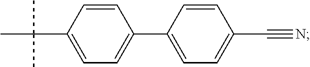

(iii) when R¹ is

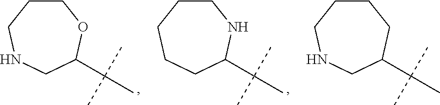

and L is

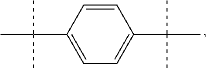

then R² is not

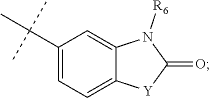

or
(iv) when R¹ is

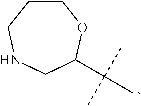

and L is

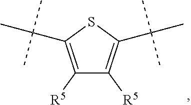

then R² is not

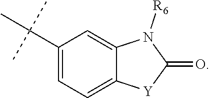

2. The compound of embodiment 1, or a pharmaceutically acceptable salt thereof, wherein, R¹ is

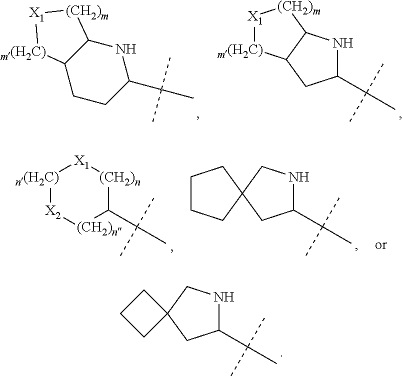

3. The compound of embodiment 1, or a pharmaceutically acceptable salt thereof, wherein R¹ is

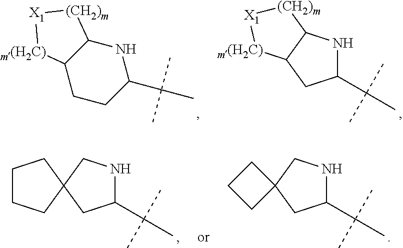

4. The compound of embodiment 1, or a pharmaceutically acceptable salt thereof, wherein R¹ is

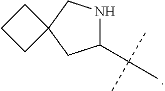

5. The compound of embodiment 1, or a pharmaceutically acceptable salt thereof, wherein R¹ is

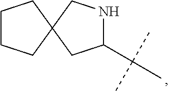

6. The compound of embodiment 1, or a pharmaceutically acceptable salt thereof, wherein R¹ is

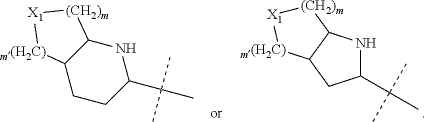

7. The compound of embodiment 1, or a pharmaceutically acceptable salt thereof, wherein R¹ is

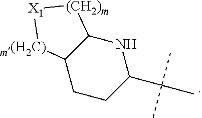

8. The compound of embodiment 1, or a pharmaceutically acceptable salt thereof, wherein R¹ is

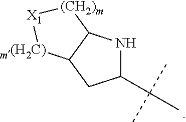

9. The compound of any one of embodiments 6-8, or a pharmaceutically acceptable salt thereof, wherein m is 1 and m′ is 1.
10. The compound of any one of embodiments 6-8, or a pharmaceutically acceptable salt thereof, wherein m is 1 and m′ is 2.
11. The compound of any one of embodiments 6-8, or a pharmaceutically acceptable salt thereof, wherein m is 2 and m′ is 1.
12. The compound of any one of embodiments 6-8, or a pharmaceutically acceptable salt thereof, wherein m is 1 and m′ is 0.
13. The compound of any one of embodiments 6-8, or a pharmaceutically acceptable salt thereof, wherein m is 0 and m′ is 1.
14. The compound of any one of embodiments 6-8, or a pharmaceutically acceptable salt thereof, wherein m is 2 and m′ is 0.
15. The compound of any one of embodiments 6-8, or a pharmaceutically acceptable salt thereof, wherein m is 0 and m′ is 2.
16. The compound of any one of embodiments 6-8, or a pharmaceutically acceptable salt thereof, wherein m is 0 and m′ is 0.
17. The compound of any one of embodiments 6-16, or a pharmaceutically acceptable salt thereof, wherein X₁ is O.
18. The compound of any one of embodiments 6-16, or a pharmaceutically acceptable salt thereof, wherein X₁ is S.
19. The compound of any one of embodiments 6-16, or a pharmaceutically acceptable salt thereof, wherein X₁ is NH.
20. The compound of any one of embodiments 6-16, or a pharmaceutically acceptable salt thereof, wherein X₁ is CH₂.
21. The compound of embodiment 1, or a pharmaceutically acceptable salt thereof, wherein R¹ is

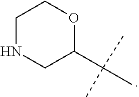

22. The compound of embodiment 1, or a pharmaceutically acceptable salt thereof, wherein R¹ is

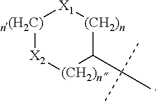

23. The compound of embodiment 22, or a pharmaceutically acceptable salt thereof, wherein the sum of n, n′ and n″ is 3.
24. The compound of embodiment 22, or a pharmaceutically acceptable salt thereof, wherein the sum of n, n′ and n″ is 2.
25. The compound of embodiment 22, or a pharmaceutically acceptable salt thereof, wherein the sum of n, n′ and n″ is 1.
26. The compound of embodiment 22, or a pharmaceutically acceptable salt thereof, wherein the sum of n, n′ and n″ is 0.
27. The compound of embodiment 23, or a pharmaceutically acceptable salt thereof, wherein n is 0, n′ is 0 and n″ is 3.
28. The compound of embodiment 23, or a pharmaceutically acceptable salt thereof, wherein n is 0, n′ is 3 and n″ is 0.
29 The compound of embodiment 23, or a pharmaceutically acceptable salt thereof, wherein n is 3, n′ is 0 and n″ is 0.
30. The compound of embodiment 23, or a pharmaceutically acceptable salt thereof, wherein n is 0, n′ is 1 and n″ is 2.
31. The compound of embodiment 23, or a pharmaceutically acceptable salt thereof, wherein n is 0, n′ is 2 and n″ is 1.
32. The compound of embodiment 23, or a pharmaceutically acceptable salt thereof, wherein n is 2, n′ is 1 and n″ is 0.
33. The compound of embodiment 23, or a pharmaceutically acceptable salt thereof, wherein n is 2, n′ is 0 and n″ is 1.
34. The compound of embodiment 24, or a pharmaceutically acceptable salt thereof, wherein n is 2, n′ is 0 and n″ is 0.
35. The compound of embodiment 24, or a pharmaceutically acceptable salt thereof, wherein n is 0, n′ is 2 and n″ is 0.
36. The compound of embodiment 24, or a pharmaceutically acceptable salt thereof, wherein n is 0, n′ is 0 and n″ is 2.
37. The compound of embodiment 24, or a pharmaceutically acceptable salt thereof, wherein n is 1, n′ is 1 and n″ is 0.
38. The compound of embodiment 24, or a pharmaceutically acceptable salt thereof, wherein n is 1, n′ is 0 and n″ is 1.
39. The compound of embodiment 24, or a pharmaceutically acceptable salt thereof, wherein n is 0, n′ is 1 and n″ is 1.
40. The compound of embodiment 25, or a pharmaceutically acceptable salt thereof, wherein n is 0, n′ is 1 and n″ is 0.
41. The compound of embodiment 25, or a pharmaceutically acceptable salt thereof, wherein n is 0, n′ is 0 and n″ is 1.
42. The compound of embodiment 25, or a pharmaceutically acceptable salt thereof, wherein n is 1, n′ is 0s and n″ is 0.
43. The compound of any one of embodiments 1-42, or a pharmaceutically acceptable salt thereof, wherein,
R² is

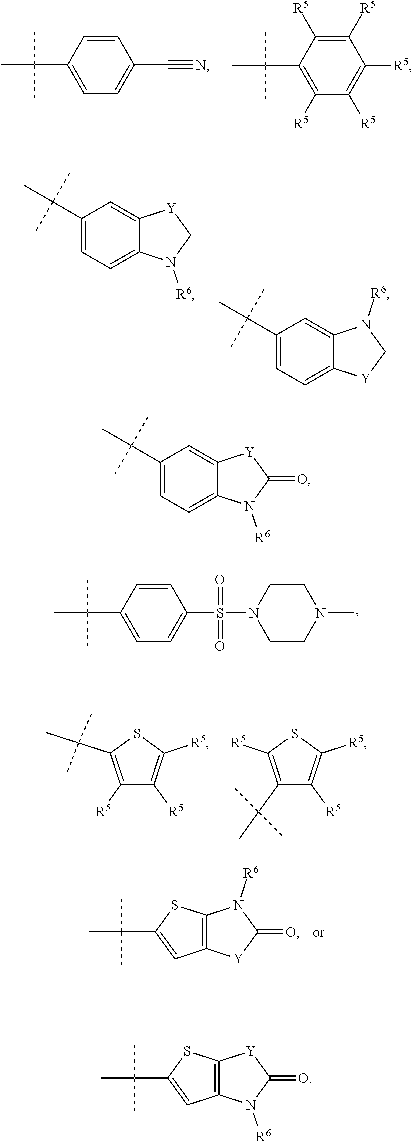

44. The compound of any one of embodiments 1-42, or a pharmaceutically acceptable salt thereof, wherein R² is

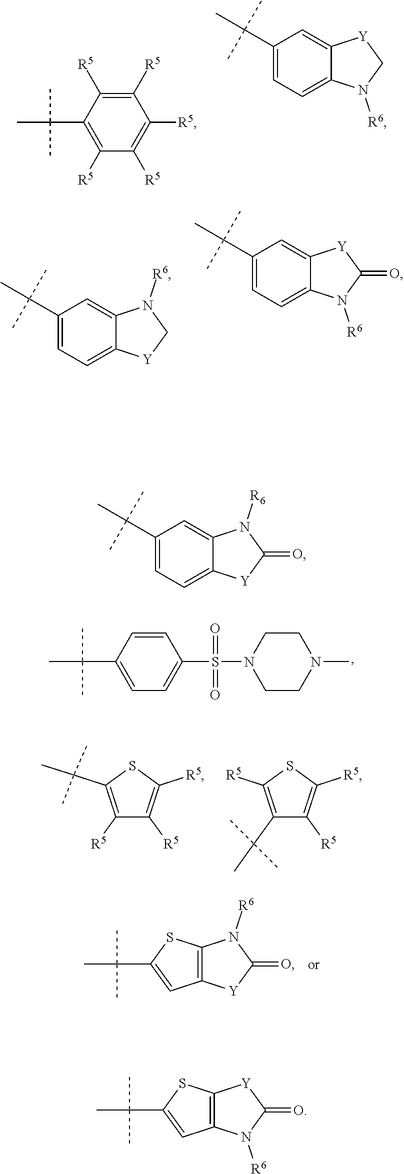

45. The compound of any one of embodiments 1-42, or a pharmaceutically acceptable salt thereof, wherein R² is

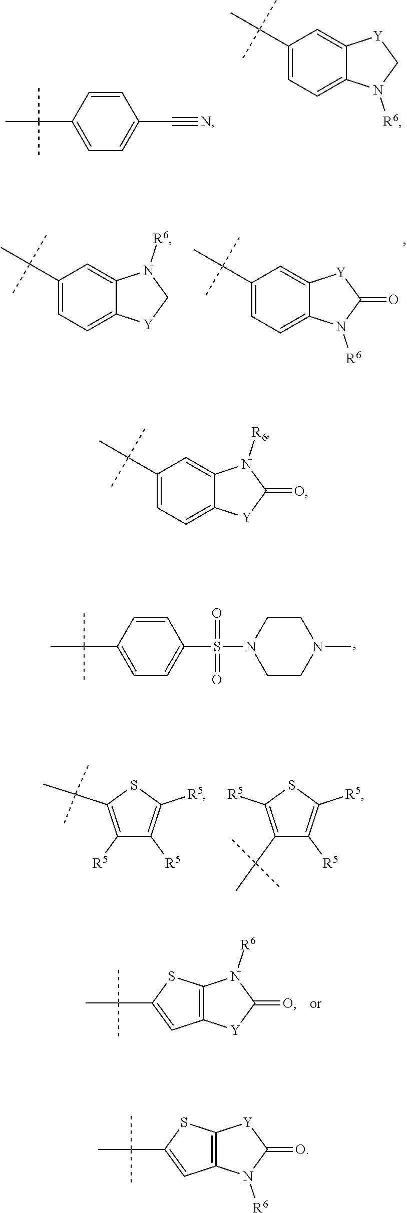

46. The compound of any one of embodiments 1-42, or a pharmaceutically acceptable salt thereof, wherein R² is

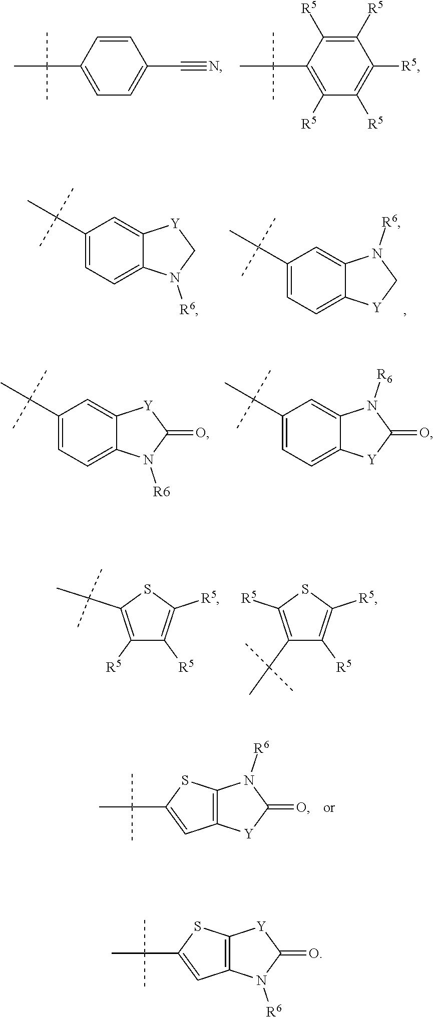

47. The compound of any one of embodiments 1-42, or a pharmaceutically acceptable salt thereof, wherein R² is

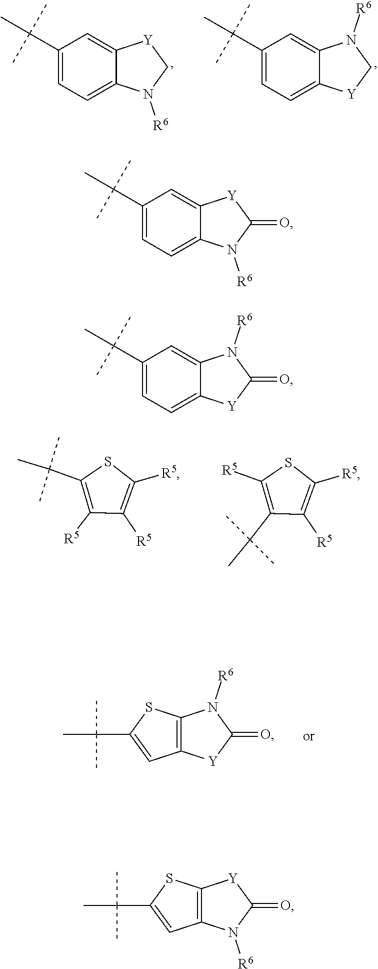

48. The compound of any one of embodiments 1-42, or a pharmaceutically acceptable salt thereof, wherein R² is

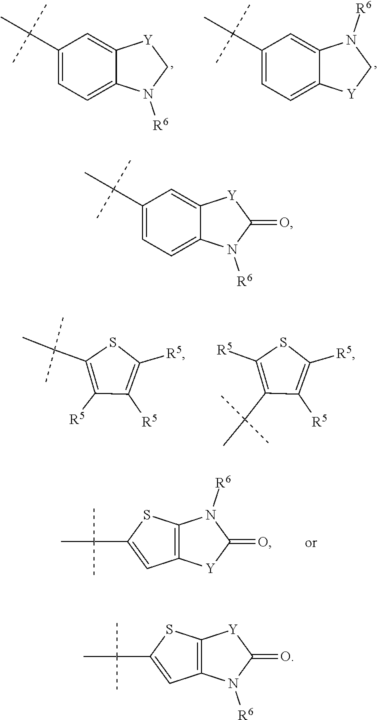

49. The compound of any one of embodiments 1-42, or a pharmaceutically acceptable salt thereof, wherein R² is

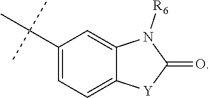

50. The compound of any one of embodiments 1-42, or a pharmaceutically acceptable salt thereof, wherein R² is

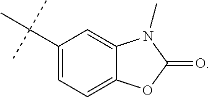

51. The compound of any one of embodiments 1-42, or a pharmaceutically acceptable salt thereof, wherein R² is

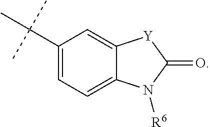

52. The compound of any one of embodiments 1-42, or a pharmaceutically acceptable salt thereof, wherein R² is

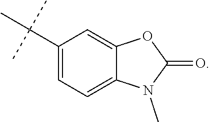

53. The compound of any one of embodiments 1-42, or a pharmaceutically acceptable salt thereof, wherein R² is

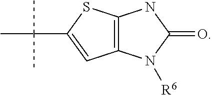

54. The compound of any one of embodiments 1-42, or a pharmaceutically acceptable salt thereof, wherein R² is

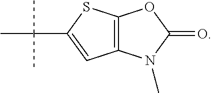

55. The compound of any one of embodiments 1-42, or a pharmaceutically acceptable salt thereof, wherein R² is

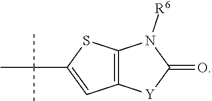

56. The compound of any one of embodiments 1-42, or a pharmaceutically acceptable salt thereof, wherein R² is

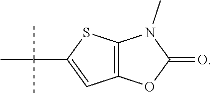

57. The compound of any one of embodiments 1-42, or a pharmaceutically acceptable salt thereof, wherein R² is

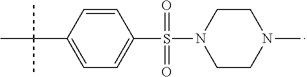

58. The compound of any one of embodiments 1-42, or a pharmaceutically acceptable salt thereof, wherein R² is

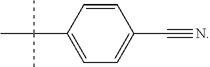

59. The compound of any one of embodiments 1-58, or a pharmaceutically acceptable salt thereof, wherein
L is

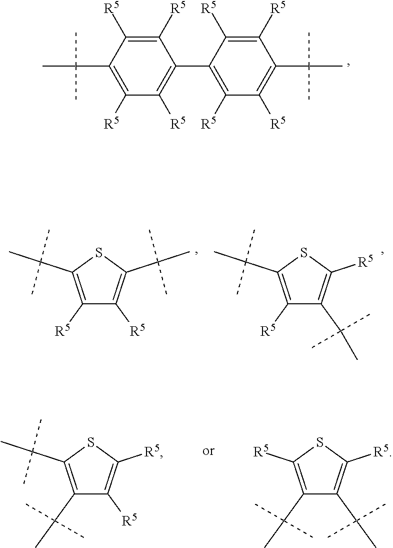

60. The compound of any one of embodiments 1-58, or a pharmaceutically acceptable salt thereof, wherein
L is

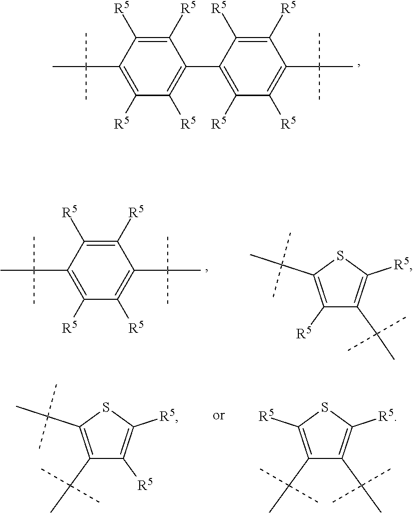

61. The compound of any one of embodiments 1-58, or a pharmaceutically acceptable salt thereof, wherein
L is

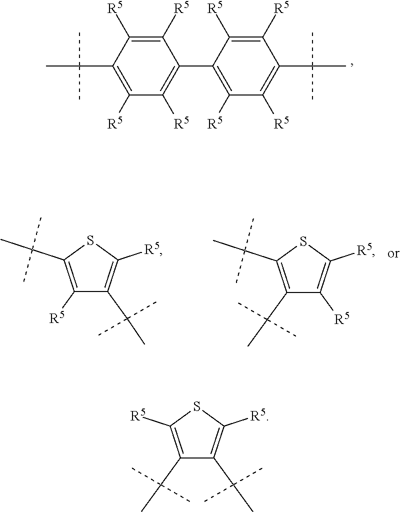

62. The compound of any one of embodiments 1-20, 22-48 and 51-57, or a pharmaceutically acceptable salt thereof, wherein L is

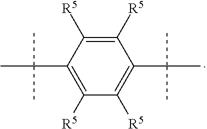

63. The compound of any one of embodiments 1-20, 22-48 and 51-57, or a pharmaceutically acceptable salt thereof, wherein L is

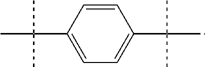

64. The compound of any one of embodiments 1-20, 22-48 and 51-57, or a pharmaceutically acceptable salt thereof, wherein L is

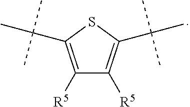

65. The compound of any one of embodiments 1-20, 22-48 and 51-57, or a pharmaceutically acceptable salt thereof, wherein L is

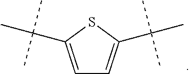

66. The compound of any one of embodiments 1-58, or a pharmaceutically acceptable salt thereof, wherein L is

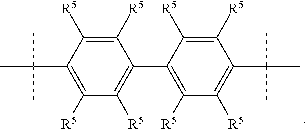

67. The compound of any one of embodiments 1-58, or a pharmaceutically acceptable salt thereof, wherein L is

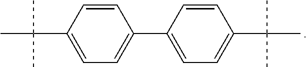

68. The compound of embodiment 1, selected from one of the compounds of Table 3.
69. A compound of Formula (II), or a pharmaceutically acceptable salt thereof:

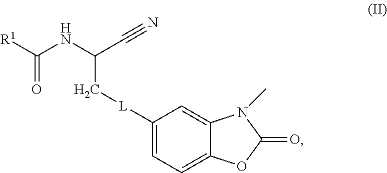

wherein,
R¹ is

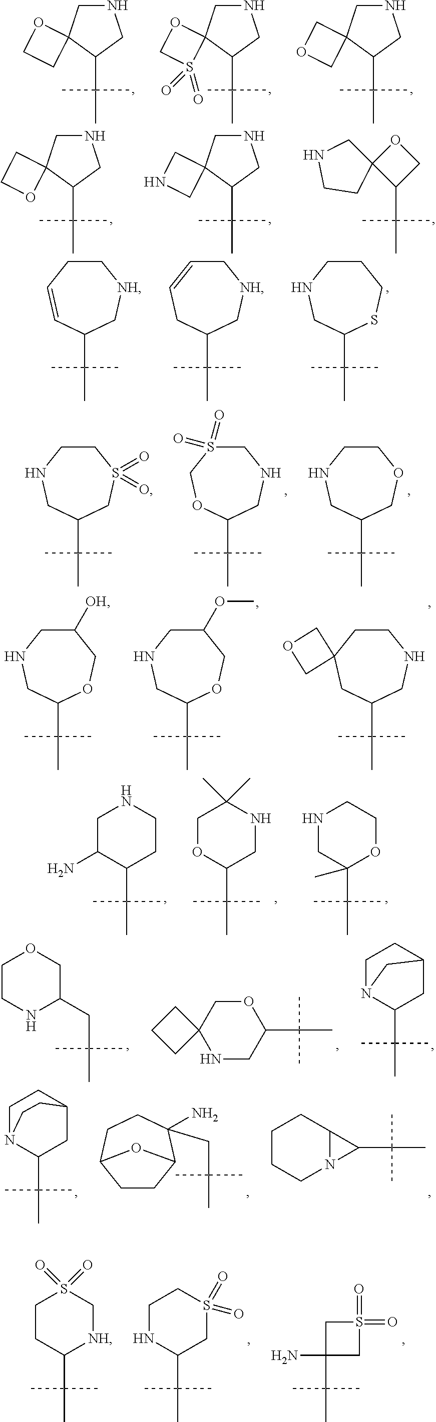

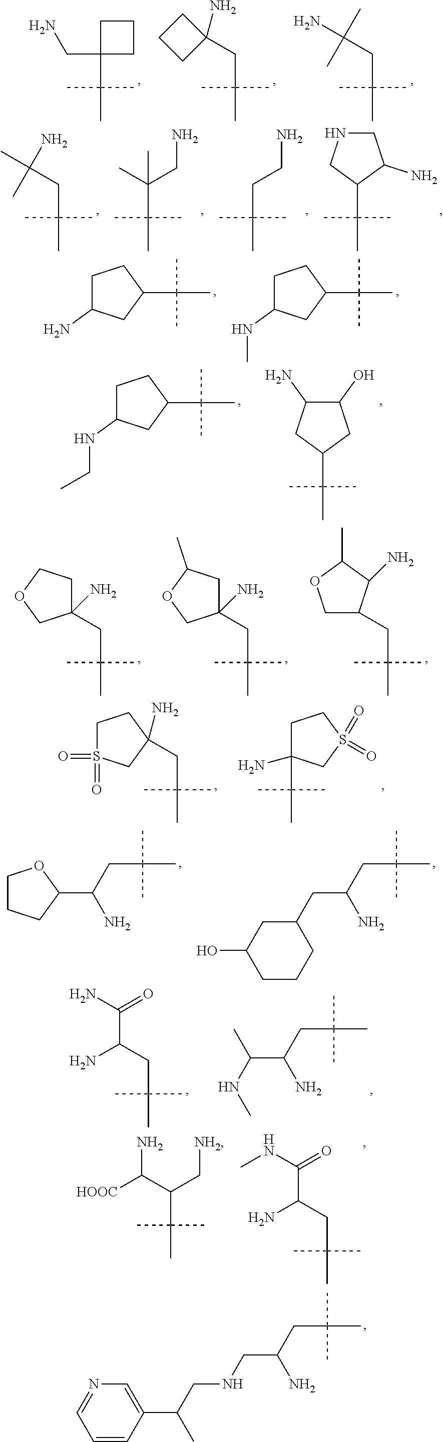

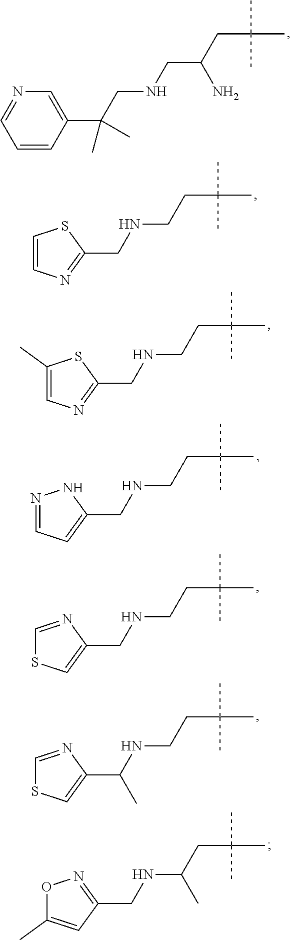

Each R² is independently H, F, Cl, Br, I or C₁-C₆ alkyl; and
L is

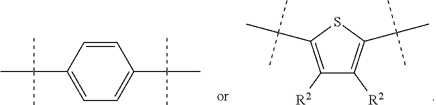

70. The compound of embodiment 48, or a pharmaceutically acceptable salt thereof, wherein L is

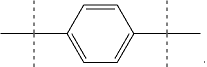

71. The compound of embodiment 48, or a pharmaceutically acceptable salt thereof, wherein L is

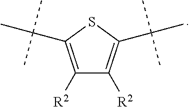

72. The compound of embodiment 48, or a pharmaceutically acceptable salt thereof, wherein L is

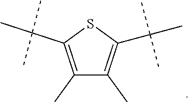

73. The compound of embodiment 48, or a pharmaceutically acceptable salt thereof, wherein L is

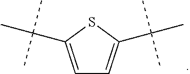

74. The compound of any one of embodiments 69-73, or a pharmaceutically acceptable salt thereof, wherein R¹ is

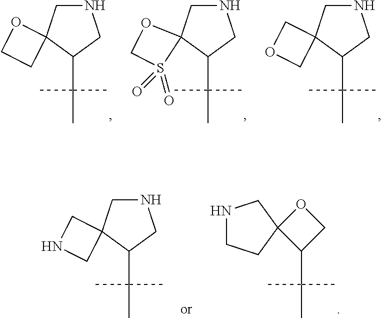

75. The compound of any one of embodiments 69-73, or a pharmaceutically acceptable salt thereof, wherein R¹ is

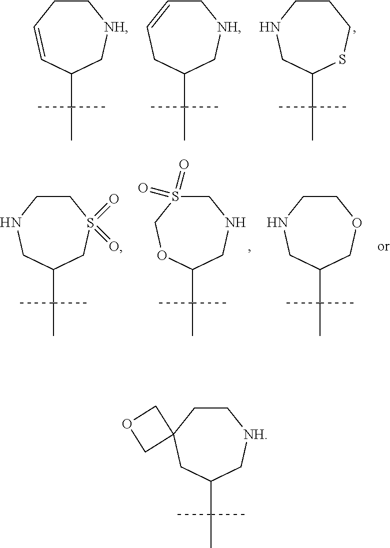

76. The compound of any one of embodiments 69-73, or a pharmaceutically acceptable salt thereof, wherein R¹ is

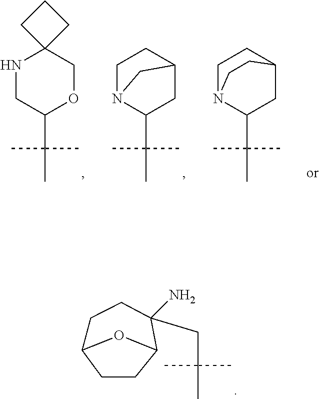

77. The compound of any one of embodiments 69-73, or a pharmaceutically acceptable salt thereof, wherein R¹ is

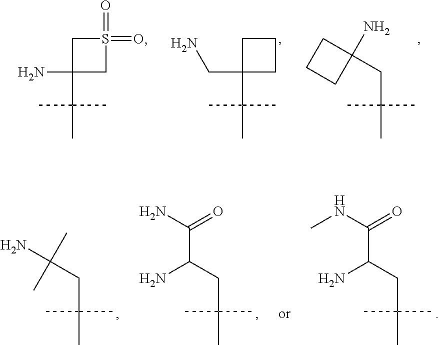

78. The compound of any one of embodiments 69-73, or a pharmaceutically acceptable salt thereof, wherein R¹ is

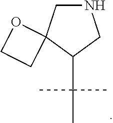

79. The compound of any one of embodiments 69-73, or a pharmaceutically acceptable salt thereof, wherein R¹ is

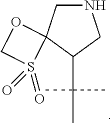

80. The compound of any one of embodiments 69-73, or a pharmaceutically acceptable salt thereof, wherein R¹ is

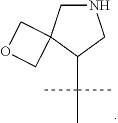

81. The compound of any one of embodiments 69-73, or a pharmaceutically acceptable salt thereof, wherein R¹ is

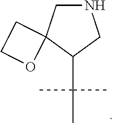

82. The compound of any one of embodiments 69-73, or a pharmaceutically acceptable salt thereof, wherein R¹ is

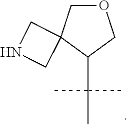

83. The compound of any one of embodiments 69-73, or a pharmaceutically acceptable salt thereof, wherein R¹ is

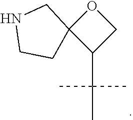

84. The compound of any one of embodiments 69-73, or a pharmaceutically acceptable salt thereof, wherein R¹ is

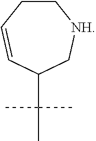

85. The compound of any one of embodiments 69-73, or a pharmaceutically acceptable salt thereof, wherein R¹ is

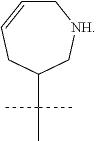

86. The compound of any one of embodiments 69-73, or a pharmaceutically acceptable salt thereof, wherein R¹ is

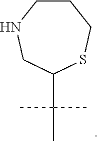

87. The compound of any one of embodiments 69-73, or a pharmaceutically acceptable salt thereof, wherein R¹ is

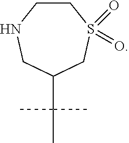

88. The compound of any one of embodiments 69-73, or a pharmaceutically acceptable salt thereof, wherein R¹ is

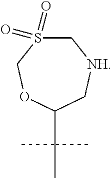

89. The compound of any one of embodiments 69-73, or a pharmaceutically acceptable salt thereof, wherein R¹ is

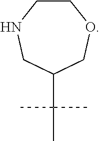

90. The compound of any one of embodiments 69-73, or a pharmaceutically acceptable salt thereof, wherein R¹ is

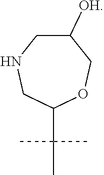

91. The compound of any one of embodiments 69-73, or a pharmaceutically acceptable salt thereof, wherein R¹ is

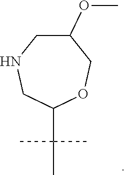

92. The compound of any one of embodiments 69-73, or a pharmaceutically acceptable salt thereof, wherein R¹ is

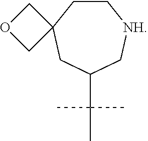

93. The compound of any one of embodiments 69-73, or a pharmaceutically acceptable salt thereof, wherein R¹ is

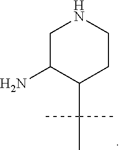

94. The compound of any one of embodiments 69-73, or a pharmaceutically acceptable salt thereof, wherein R¹ is

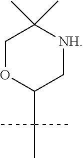

95. The compound of any one of embodiments 69-73, or a pharmaceutically acceptable salt thereof, wherein R¹ is

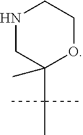

96. The compound of any one of embodiments 69-73, or a pharmaceutically acceptable salt thereof, wherein R¹ is

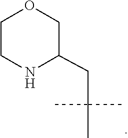

97. The compound of any one of embodiments 69-73, or a pharmaceutically acceptable salt thereof, wherein R¹ is

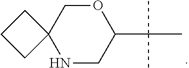

98. The compound of any one of embodiments 69-73, or a pharmaceutically acceptable salt thereof, wherein R¹ is

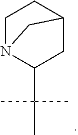

99. The compound of any one of embodiments 69-73, or a pharmaceutically acceptable salt thereof, wherein R¹ is

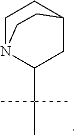

100. The compound of any one of embodiments 69-73, or a pharmaceutically acceptable salt thereof, wherein R¹ is

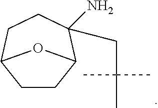

101. The compound of any one of embodiments 69-73, or a pharmaceutically acceptable salt thereof, wherein R¹ is

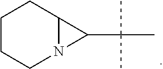

102. The compound of any one of embodiments 69-73, or a pharmaceutically acceptable salt thereof, wherein R¹ is

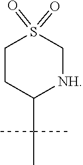

103. The compound of any one of embodiments 69-73, or a pharmaceutically acceptable salt thereof, wherein R¹ is

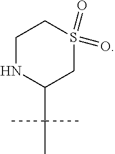

104. The compound of any one of embodiments 69-73, or a pharmaceutically acceptable salt thereof, wherein R¹ is

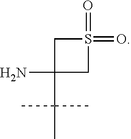

105. The compound of any one of embodiments 69-73, or a pharmaceutically acceptable salt thereof, wherein R¹ is

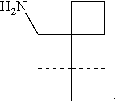

106. The compound of any one of embodiments 69-73, or a pharmaceutically acceptable salt thereof, wherein R¹ is

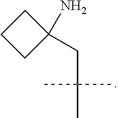

107. The compound of any one of embodiments 69-73, or a pharmaceutically acceptable salt thereof, wherein R¹ is

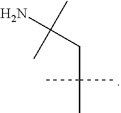

108. The compound of any one of embodiments 69-73, or a pharmaceutically acceptable salt thereof, wherein R¹ is

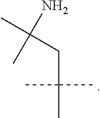

109. The compound of any one of embodiments 69-73, or a pharmaceutically acceptable salt thereof, wherein R¹ is

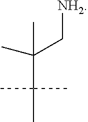

110. The compound of any one of embodiments 69-73, or a pharmaceutically acceptable salt thereof, wherein R¹ is

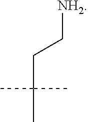

111. The compound of any one of embodiments 69-73, or a pharmaceutically acceptable salt thereof, wherein R¹ is

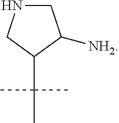

112. The compound of any one of embodiments 69-73, or a pharmaceutically acceptable salt thereof, wherein R¹ is

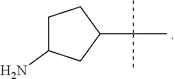

113. The compound of any one of embodiments 69-73, or a pharmaceutically acceptable salt thereof, wherein R¹ is

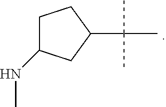

114. The compound of any one of embodiments 69-73, or a pharmaceutically acceptable salt thereof, wherein R¹ is

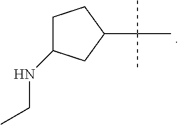

115. The compound of any one of embodiments 69-73, or a pharmaceutically acceptable salt thereof, wherein R¹ is

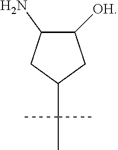

116. The compound of any one of embodiments 69-73, or a pharmaceutically acceptable salt thereof, wherein R¹ is

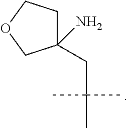

117. The compound of any one of embodiments 69-73, or a pharmaceutically acceptable salt thereof, wherein R¹ is

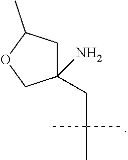

118. The compound of any one of embodiments 69-73, or a pharmaceutically acceptable salt thereof, wherein R¹ is

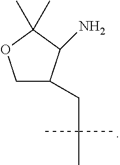

119. The compound of any one of embodiments 69-73, or a pharmaceutically acceptable salt thereof, wherein R¹ is

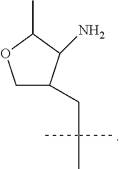

120. The compound of any one of embodiments 69-73, or a pharmaceutically acceptable salt thereof, wherein R¹ is

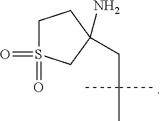

121. The compound of any one of embodiments 69-73, or a pharmaceutically acceptable salt thereof, wherein R¹ is

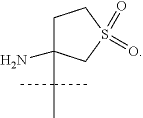

122. The compound of any one of embodiments 69-73, or a pharmaceutically acceptable salt thereof, wherein R¹ is

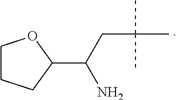

123. The compound of any one of embodiments 69-73, or a pharmaceutically acceptable salt thereof, wherein R¹ is

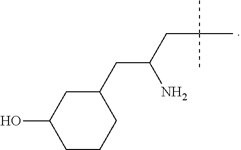

124. The compound of any one of embodiments 69-73, or a pharmaceutically acceptable salt thereof, wherein R¹ is

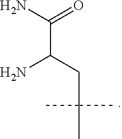

125. The compound of any one of embodiments 69-73, or a pharmaceutically acceptable salt thereof, wherein R¹ is

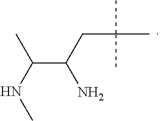

126. The compound of any one of embodiments 69-73, or a pharmaceutically acceptable salt thereof, wherein R¹ is

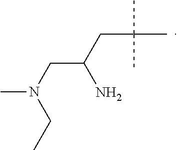

127. The compound of any one of embodiments 69-73, or a pharmaceutically acceptable salt thereof, wherein R¹ is

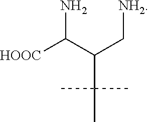

128. The compound of any one of embodiments 69-73, or a pharmaceutically acceptable salt thereof, wherein R¹ is

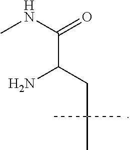

129. The compound of any one of embodiments 69-73, or a pharmaceutically acceptable salt thereof, wherein R¹ is

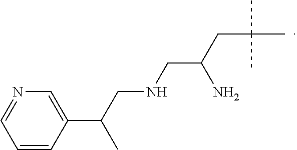

130. The compound of any one of embodiments 69-73, or a pharmaceutically acceptable salt thereof, wherein R¹ is

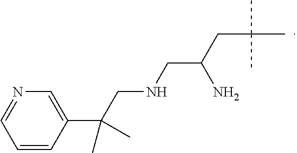

131. The compound of any one of embodiments 69-73, or a pharmaceutically acceptable salt thereof, wherein R¹ is

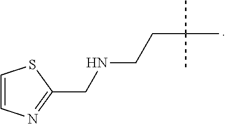

132. The compound of any one of embodiments 69-73, or a pharmaceutically acceptable salt thereof, wherein R¹ is

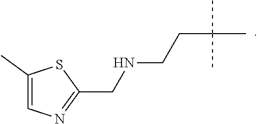

133. The compound of any one of embodiments 69-73, or a pharmaceutically acceptable salt thereof, wherein R¹ is

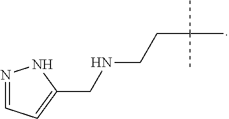

134. The compound of any one of embodiments 69-73, or a pharmaceutically acceptable salt thereof, wherein R¹ is

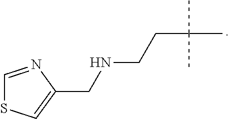

135. The compound of any one of embodiments 69-73, or a pharmaceutically acceptable salt thereof, wherein R¹ is

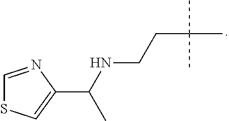

136. The compound of any one of embodiments 69-73, or a pharmaceutically acceptable salt thereof, wherein R¹ is

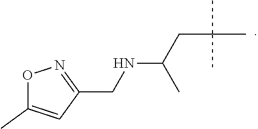

137. A compound of Formula (III), or a pharmaceutically acceptable salt thereof:

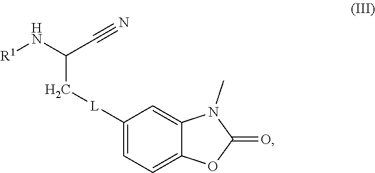

wherein,
R¹ is

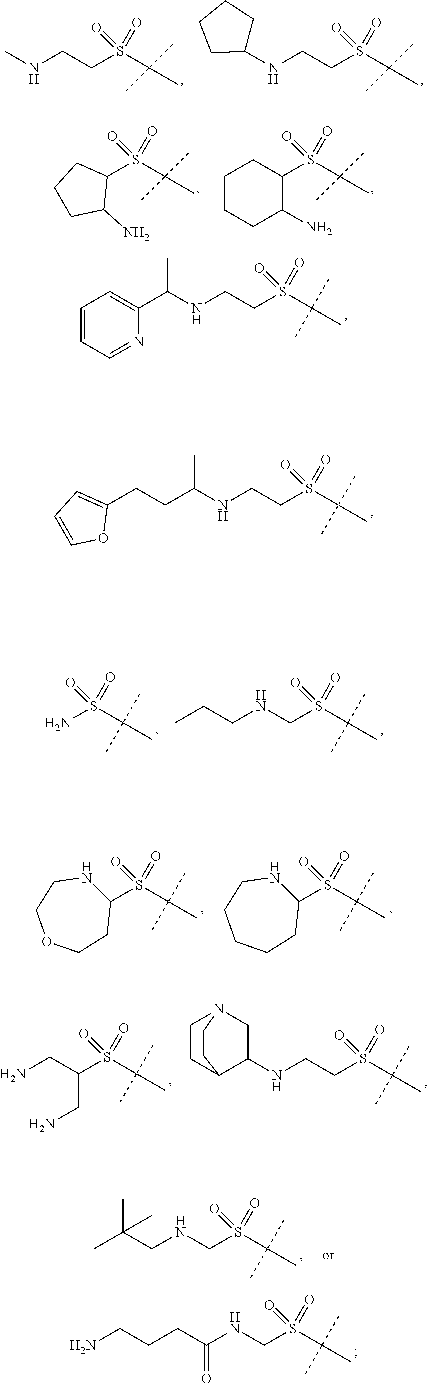

Each R² is independently H, F, Cl, Br, I or C₁-C₆ alkyl; and

L is

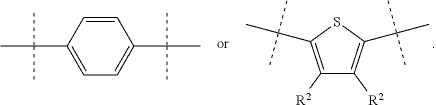

138. The compound of embodiment 116, or a pharmaceutically acceptable salt thereof, wherein L is

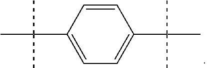

139. The compound of embodiment 116, or a pharmaceutically acceptable salt thereof, wherein L is

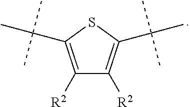

140. The compound of embodiment 116, or a pharmaceutically acceptable salt thereof, wherein L is

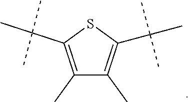

141. The compound of embodiment 116, or a pharmaceutically acceptable salt thereof, wherein L is

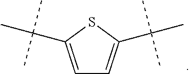

142. The compound of any one of embodiments 137-141, or a pharmaceutically acceptable salt thereof, wherein R¹ is

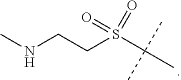

143. The compound of any one of embodiments 137-141, or a pharmaceutically acceptable salt thereof, wherein R¹ is

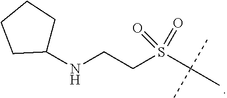

144. The compound of any one of embodiments 137-141, or a pharmaceutically acceptable salt thereof, wherein R¹ is

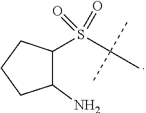

145. The compound of any one of embodiments 137-141, or a pharmaceutically acceptable salt thereof, wherein R¹ is

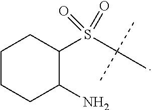

146. The compound of any one of embodiments 137-141, or a pharmaceutically acceptable salt thereof, wherein R¹ is

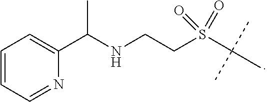

147. The compound of any one of embodiments 137-141, or a pharmaceutically acceptable salt thereof, wherein R¹ is

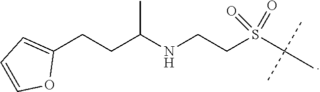

148. The compound of any one of embodiments 137-141, or a pharmaceutically acceptable salt thereof, wherein R¹ is

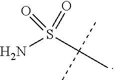

149. The compound of any one of embodiments 137-141, or a pharmaceutically acceptable salt thereof, wherein R¹ is

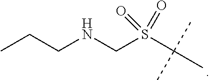

150. The compound of any one of embodiments 137-141, or a pharmaceutically acceptable salt thereof, wherein R¹ is

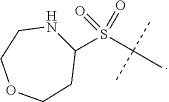

151. The compound of any one of embodiments 137-141, or a pharmaceutically acceptable salt thereof, wherein R¹ is

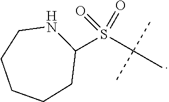

152. The compound of any one of embodiments 137-141, or a pharmaceutically acceptable salt thereof, wherein R¹ is

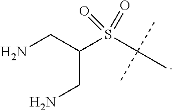

153. The compound of any one of embodiments 137-141, or a pharmaceutically acceptable salt thereof, wherein R¹ is

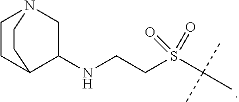

154. The compound of any one of embodiments 137-141, or a pharmaceutically acceptable salt thereof, wherein R¹ is

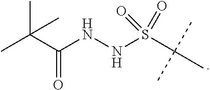

155. The compound of any one of embodiments 137-141, or a pharmaceutically acceptable salt thereof, wherein R¹ is

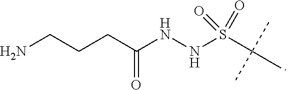

156. A method for treating an obstructive disease of the airway in a patient in need thereof, comprising, administering to the patient for an administration period, a composition comprising an effective amount of a compound of any one of embodiments 1-155.
157. The method of embodiment 124, wherein the obstructive disease of the airway is asthma, chronic obstructive pulmonary disease (COPD), bronchitis, emphysema, cystic fibrosis (CF), bronchiectasis, sarcoidosis; alpha-1 antitrypsin (A1AT) deficiency, farmer's lung and related diseases, hypersensitivity pneumonitis, lung fibrosis, complications of lung transplantation, vasculitic and thrombotic disorders of the lung vasculature, pulmonary hypertension, antitussive activity including treatment of chronic cough associated with inflammatory and secretory conditions of the airways, iatrogenic cough, acute and chronic rhinitis including rhinitis medicamentosa, and vasomotor rhinitis; perennial and seasonal allergic rhinitis including rhinitis nervosa (hay fever), nasal polyposis; acute viral infection including the common cold, and infection due to a respiratory virus, acute lung injury, or acute respiratory distress syndrome (ARDS).
158. The method of embodiment 157, wherein the obstructive disease of the airway is asthma.
159. The method of embodiment 157, wherein the obstructive disease of the airway is acute respiratory distress syndrome (ARDS).
160. The method of embodiment 157, wherein the obstructive disease of the airway is bronchitis.
161. The method of embodiment 157, wherein the obstructive disease of the airway is lung fibrosis.
162. The method of embodiment 157, wherein the obstructive disease of the airway is emphysema.
163. The method of embodiment 157, wherein the obstructive disease of the airway is cystic fibrosis (CF).
164. The method of embodiment 157, wherein the obstructive disease of the airway is bronchiectasis.
165. The method of embodiment 157, wherein the obstructive disease of the airway is sarcoidosis.
166. The method of embodiment 157, wherein the obstructive disease of the airway is alpha-1 antitrypsin (A1AT) deficiency.
167. The method of embodiment 157, wherein the obstructive disease of the airway is farmer's lung.
168. The method of embodiment 157, wherein the obstructive disease of the airway is hypersensitivity pneumonitis.
169. The method of embodiment 157, wherein the obstructive disease of the airway is a complication of lung transplantation.
170. The method of embodiment 157, wherein the obstructive disease of the airway is a vasculitic or thrombotic disorder of the lung vasculature.
171. The method of embodiment 157, wherein the obstructive disease of the airway is pulmonary hypertension.
172. The method of embodiment 157, wherein the obstructive disease of the airway is iatrogenic cough.
173. The method of embodiment 157, wherein the obstructive disease of the airway is iatrogenic cough.
174. The method of embodiment 157, wherein the obstructive disease of the airway is acute rhinitis.
175. The method of embodiment 157, wherein the obstructive disease of the airway is chronic rhinitis.
176. The method of embodiment 157, wherein the obstructive disease of the airway is nasal polyposis.
177. The method of embodiment 158, wherein the asthma is bronchial, allergic, intrinsic, extrinsic, exercise-induced or drug-induced asthma.
178. The method of embodiment 160, wherein the bronchitis is infectious bronchitis or eosinophilic bronchitis.
179. The method of embodiment 161, wherein the lung fibrosis is idiopathic pulmonary fibrosis, cryptogenic fibrosing alveolitis, idiopathic interstitial pneumonia, or fibrosis complicating anti-neoplastic therapy or chronic infection.
180. The method of embodiment 164, wherein the bronchiectasis is non-cystic fibrosis bronchiectasis (NCFBE).
181. The method of embodiment 164, wherein the bronchiectasis is associated with cystic fibrosis.
182. The method of embodiment 171, wherein the pulmonary hypertension is pulmonary arterial hypertension.
182. A method for treating cystic fibrosis in a patient in need thereof, comprising, administering to the patient for an administration period, a composition comprising an effective amount of a compound of any one of embodiments 1-155.
183. The method of embodiment 182, wherein the treating comprises improving the lung function of the patient, as compared to the lung function of the patient prior to the administration period.
184. The method of embodiment 183, wherein improving lung function of the patient comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75%)}), as compared to the respective value for the patient prior to the administration period.
185. The method of embodiment 183 or 184, wherein the lung function is measured by spirometry.
186. A method for treating bronchiectasis in a patient in need thereof, comprising, administering to the patient for an administration period, a composition comprising an effective amount of a compound of any one of embodiments 1-155.
187. The method of embodiment 186, wherein the bronchiectasis is non-cystic fibrosis bronchiectasis (NCFBE).
188. The method of embodiment 186, wherein the bronchiectasis is associated with cystic fibrosis.
189. The method of any one of embodiments 186-188, wherein treating comprises improving the lung function of the patient, as compared to the lung function of the patient prior to the administration period.
190. The method of embodiment 189, wherein improving lung function of the patient comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75%)}), as compared to the respective value for the patient prior to the administration period.
191. The method of embodiment 189 or 190, wherein the lung function is measured by spirometry.
192. The method of any one of embodiments 186-191, wherein treating comprises decreasing the rate of pulmonary exacerbation, as compared to the rate of pulmonary exacerbation of the patient prior to the administration period.
193. The method of any one of embodiments 186-192, wherein treating comprises increasing the time to first pulmonary exacerbation, as compared to an untreated patient.
194. The method of embodiment 192 or 193, wherein the pulmonary exacerbation is characterized by three or more of the following symptoms exhibited for at least 48 hours by the patient: (1) increased cough; (2) increased sputum volume or change in sputum consistency; (3) increased sputum purulence; (4) increased breathlessness and/or decreased exercise tolerance; (5) fatigue and/or malaise; (6) hemoptysis.
195. A method for treating chronic rhinosinusitis (CRS) in a patient in need thereof, comprising, administering to the patient for an administration period, a composition comprising an effective amount of a compound of any one of embodiments 1-155.
196. The method of embodiment 195, wherein the chronic rhinosinusitis is chronic rhinosinusitis without nasal polyps (CRSsNP).
197. The method of embodiment 195, wherein the chronic rhinosinusitis is chronic rhinosinusitis with nasal polyps (CRSwNP).
198. The method of any one of embodiments 195-197, wherein the chronic rhinosinusitis is refractory chronic rhinosinusitis.
199. The method of any one of embodiments 195-198, wherein treating comprises reducing, diminishing the severity of, delaying the onset of, or eliminating one or more symptoms of CRS.
200. The method of embodiment 199, wherein the one or more symptoms of CRS is selected from nasal congestion; nasal obstruction; nasal discharge; post-nasal drip; facial pressure; facial pain; facial fullness; reduced smell; depression; mucosal edema; mucopurulent discharge; obstruction of the middle meatus; mucosal changes within the ostiomeatal complex and sinuses; or rhinorrhea.
201. A method for treating hidradenitis supporativa (HS) in a patient in need thereof, comprising, administering to the patient for an administration period, a composition comprising an effective amount of a compound of any one of embodiments 1-155.
202. The method of embodiment 201, wherein the hidradenitis supporativa (HS) is Hurley stage I.
203. The method of embodiment 201, wherein the hidradenitis supporativa (HS) is Hurley stage II.
204. The method of embodiment 201, wherein the hidradenitis supporativa (HS) is Hurley stage III.
205. A method for treating cancer in a patient in need thereof, comprising, administering to the patient for an administration period, a composition comprising an effective amount of a compound of any one of embodiments 1-155.
206. The method of embodiment 205, wherein the cancer is a metastatic cancer.
207. The method of embodiment 206, wherein the metastatic cancer is breast to lung metastatic cancer.
208. The method of embodiment 206, wherein the metastatic cancer comprises metastasis of breast cancer to the brain, bone, pancreas, lymph nodes or liver.
209. The method of embodiment 206, wherein the metastatic cancer comprises metastasis of bone cancer to the lung.
210. The method of embodiment 206, wherein the metastatic cancer comprises metastasis of colorectal cancer to the peritoneum, the pancreas, the stomach, the lung, the liver, the kidney, or the spleen.
211. The method of embodiment 206, wherein the metastatic cancer comprises metastasis of stomach cancer to the mesentery, the spleen, the pancreas, the lung, the liver, the adrenal gland, or the ovary.
212. The method of embodiment 206, wherein the metastatic cancer comprises metastasis of liver cancer to the intestine, spleen, pancreas, stomach, lung, or the kidney.
213. The method of embodiment 206, wherein the metastatic cancer comprises metastasis of lymphoma to the kidney, ovary, liver, bladder, or the spleen.
214. A method for treating lupus nephritis in a patient in need thereof, comprising, administering to the patient for an administration period, a composition comprising an effective amount of a compound of any one of embodiments 1-155.
215. A method for treating rheumatoid arthritis in a patient in need thereof, comprising, administering to the patient for an administration period, a composition comprising an effective amount of a compound of any one of embodiments 1-155.
216. A method for treating inflammatory bowel disease (IBD) in a patient in need thereof, comprising, administering to the patient for an administration period, a composition comprising an effective amount of a compound of any one of embodiments 1-155.
217. The method of embodiment 216, wherein the inflammatory bowel disease (IBD) is Crohn's disease.
218. The method of embodiment 216, wherein the inflammatory bowel disease (IBD) is ulcerative colitis.
219. The method of any one of embodiments 156-218, wherein the composition is administered once a day during the administration period.
220. The method of any one of embodiments 156-218, wherein the composition is administered twice a day during the administration period.
221. The method of any one of embodiments 156-218, wherein the composition is administered every other day during the administration period.
222. The method of any one of embodiments 156-218, wherein the composition is administered once per week during the administration period.
223. The method of any one of embodiments 156-222, wherein the composition is an oral dosage form.
224. The method of embodiment 223, wherein the composition is administered orally.
225. The method of any one of embodiments 156-224, wherein the composition comprises from about 10 mg to about 50 mg of the compound.
226. The method of any one of embodiments 156-225, wherein the administration period is from about 1 year to about 30 years.
227. The method of any one of embodiments 156-225, wherein the administration period is from about 1 year to about 20 years.
228. The method of any one of embodiments 156-225, wherein the administration period is from about 1 year to about 15 years.
229. The method of any one of embodiments 156-225, wherein the administration period is from about 1 year to about 10 years.
230. The method of any one of embodiments 156-225, wherein the administration period is from about 1 year to about 5 years.
231. The method of any one of embodiments 156-225, wherein the administration period is from about 1 year to about 5 years.
232. The method of any one of embodiments 156-225, wherein the administration period is from about 1 year to about 3 years.
233. The method of any one of embodiments 156-225, wherein the administration period is from about 2 years to about 10 years.
234. The method of any one of embodiments 156-225, wherein the administration period is from about 2 years to about 8 years.
235. The method of any one of embodiments 156-225, wherein the administration period is from about 2 years to about 5 years.
236. The method of any one of embodiments 156-225, wherein the administration period is from about 2 years to about 4 years.
237. The method of any one of embodiments 156-225, wherein the administration period is from about 2 years to about 3 years.
238. The method of any one of embodiments 156-225, wherein the administration period is at least about 30 days.
239. The method of any one of embodiments 156-225, wherein the administration period is at least about 60 days.
240. The method of any one of embodiments 156-225, wherein the administration period is at least about 90 days.
241. The method of any one of embodiments 156-225, wherein the administration period is at least about 4 months.
242. The method of any one of embodiments 156-225, wherein the administration period is at least about 6 months.
243. The method of any one of embodiments 156-225, wherein the administration period is at least about 8 months.
244. The method of any one of embodiments 156-225, wherein the administration period is at least about 10 months.
245. The method of any one of embodiments 156-225, wherein the administration period is at least about 1 year.
246. The method of any one of embodiments 156-225, wherein the administration period is at least about 2 years.
247. The method of any one of embodiments 156-225, wherein the administration period is at least about 3 years.
248. The method of any one of embodiments 156-225, wherein the administration period is at least about 4 years.
249. The method of any one of embodiments 156-225, wherein the administration period is at least about 5 years.
250. The method of any one of embodiments 156-225, wherein the administration period is at least about 10 years.

### Numbered Embodiments—Set B

1. A compound of Formula (I):

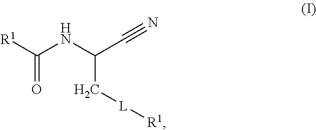

or a pharmaceutically acceptable salt or deuterated form thereof, wherein,
R¹ is

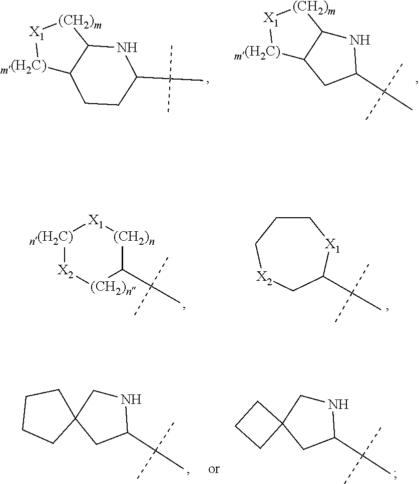

R² is

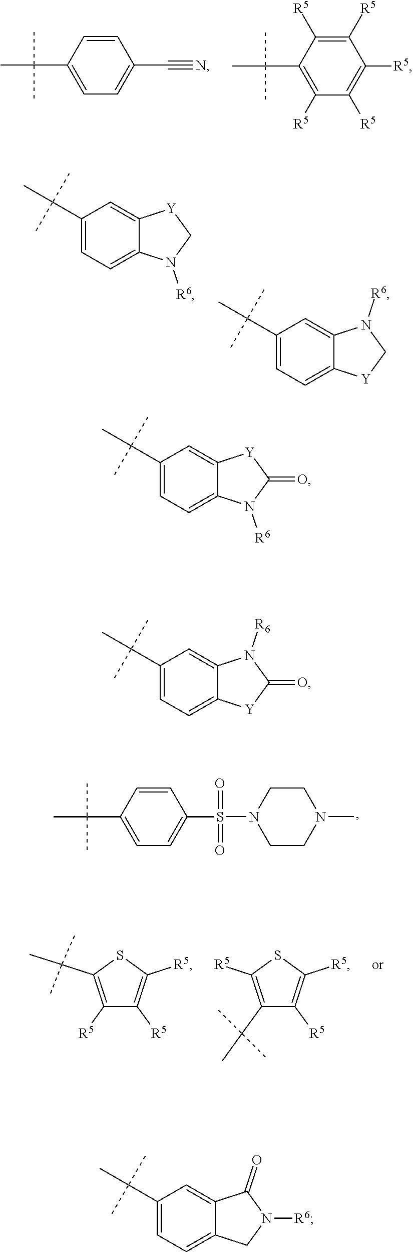

X₁, X₂ and X₃ are independently O, S, NR³ or CR³R⁴;
each R³, R⁴ and R⁵ is independently H, F, Cl, Br, I or C₁-C₆ alkyl;
each R⁶ is independently H or C₁-C₆ alkyl;
each Y is independently O, S, CHR⁶ or NR⁶;
m and m′ are each independently an integer from 0-3, and the total sum of m and m′ is ≤3;
each n, n′ and n″ is independently an integer from 0-3, and the total sum of n, n′ and n″ is ≤4; and
L is

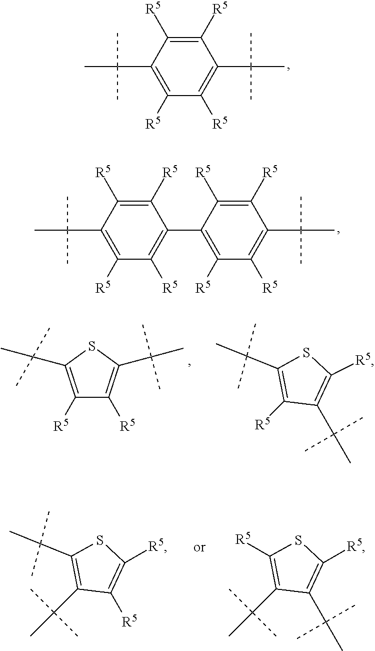

provided that,
   (i) when R¹ is

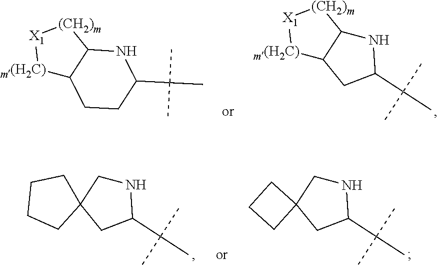

and X₁ is —CH₂—, then L and R² taken together, is not

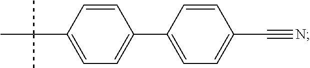

or
(ii) when R¹ is

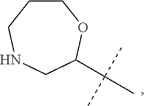

then L and R² taken together, is not

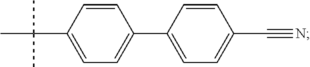

(iii) when R¹ is

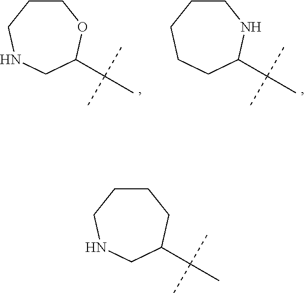

and L is

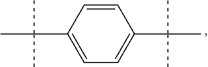

then R² is not

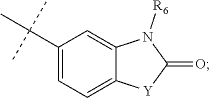

(iv) when R¹ is

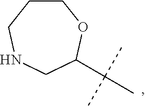

and L is

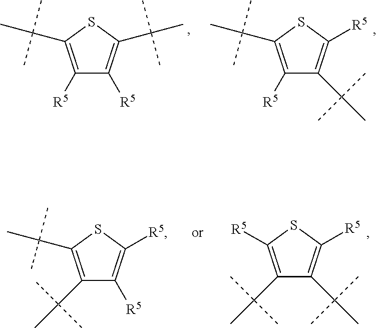

then R² is not

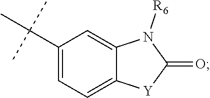

(v) when R¹ is

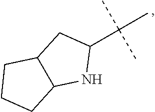

and L is

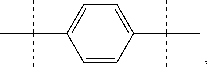

then R² is not

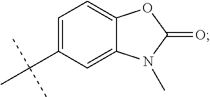

(vi) when R¹ is

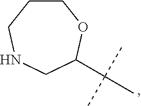

and L is

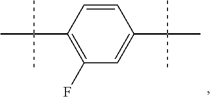

then R² is not

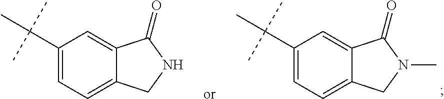

and
(vii) R¹ is

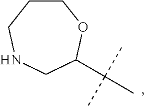

and L is

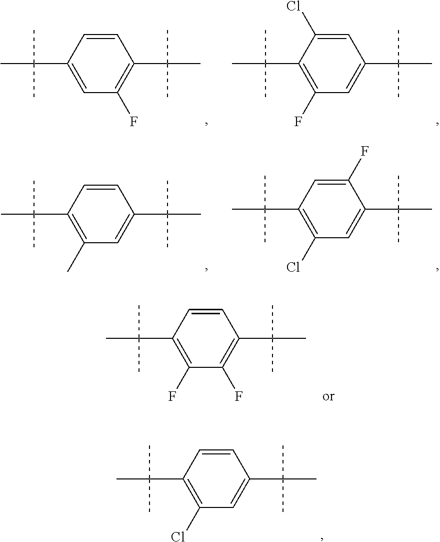

then R² is not

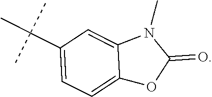

2. The compound of embodiment 1, or a pharmaceutically acceptable salt thereof or deuterated form, wherein, R¹ is

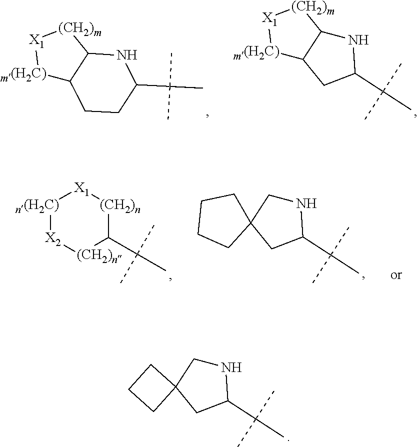

3. The compound of embodiment 1, or a pharmaceutically acceptable salt or deuterated form thereof, wherein R¹ is

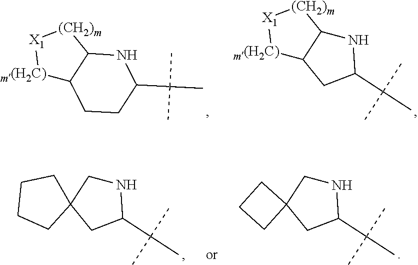

4. The compound of embodiment 1, or a pharmaceutically acceptable salt or deuterated form thereof, wherein R¹ is

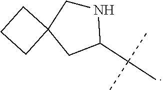

5. The compound of embodiment 1, or a pharmaceutically acceptable salt or deuterated form thereof, wherein R¹ is

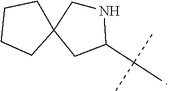

6. The compound of embodiment 1, or a pharmaceutically acceptable salt or deuterated form thereof, wherein R¹ is

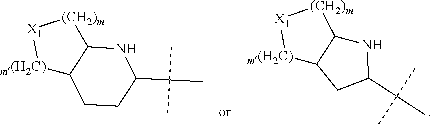

7. The compound of embodiment 1, or a pharmaceutically acceptable salt or deuterated form thereof, wherein R¹ is

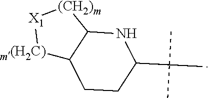

8. The compound of embodiment 1, or a pharmaceutically acceptable salt or deuterated form thereof, wherein R¹ is

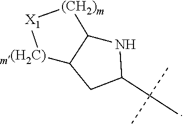

9. The compound of any one of embodiments 6-8, or a pharmaceutically acceptable salt or deuterated form thereof, wherein m is 1 and m′ is 1.
10. The compound of any one of embodiments 6-8, or a pharmaceutically acceptable salt or deuterated form thereof, wherein m is 1 and m′ is 2.
11. The compound of any one of embodiments 6-8, or a pharmaceutically acceptable salt or deuterated form thereof, wherein m is 2 and m′ is 1.
12. The compound of any one of embodiments 6-8, or a pharmaceutically acceptable salt or deuterated form thereof, wherein m is 1 and m′ is 0.
13. The compound of any one of embodiments 6-8, or a pharmaceutically acceptable salt or deuterated form thereof, wherein m is 0 and m′ is 1.
14. The compound of any one of embodiments 6-8, or a pharmaceutically acceptable salt or deuterated form thereof, wherein m is 2 and m′ is 0.
15. The compound of any one of embodiments 6-8, or a pharmaceutically acceptable salt or deuterated form thereof, wherein m is 0 and m′ is 2.
16. The compound of any one of embodiments 6-8, or a pharmaceutically acceptable salt or deuterated form thereof, wherein m is 0 and m′ is 0.
17. The compound of any one of embodiments 6-16, or a pharmaceutically acceptable salt or deuterated form thereof, wherein X₁ is O.
18. The compound of any one of embodiments 6-16, or a pharmaceutically acceptable salt or deuterated form thereof, wherein X₁ is S.
19. The compound of any one of embodiments 6-16, or a pharmaceutically acceptable salt or deuterated form thereof, wherein X₁ is NH.
20. The compound of any one of embodiments 6-16, or a pharmaceutically acceptable salt or deuterated form thereof, wherein X₁ is CH₂.
21. The compound of embodiment 1, or a pharmaceutically acceptable salt or deuterated form thereof, wherein R¹ is

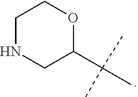

22. The compound of embodiment 1, or a pharmaceutically acceptable salt or deuterated form thereof, wherein R¹ is

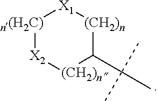

23. The compound of embodiment 22, or a pharmaceutically acceptable salt or deuterated form thereof, wherein the sum of n, n′ and n″ is 3.
24. The compound of embodiment 22, or a pharmaceutically acceptable salt or deuterated form thereof, wherein the sum of n, n′ and n″ is 2.
25. The compound of embodiment 22, or a pharmaceutically acceptable salt or deuterated form thereof, wherein the sum of n, n′ and n″ is 1.
26. The compound of embodiment 22, or a pharmaceutically acceptable salt or deuterated form thereof, wherein the sum of n, n′ and n″ is 0.
27. The compound of embodiment 23, or a pharmaceutically acceptable salt or deuterated form thereof, wherein n is 0, n′ is 0 and n″ is 3.
28. The compound of embodiment 23, or a pharmaceutically acceptable salt or deuterated form thereof, wherein n is 0, n′ is 3 and n″ is 0.
29. The compound of embodiment 23, or a pharmaceutically acceptable salt or deuterated form thereof, wherein n is 3, n′ is 0 and n″ is 0.
30. The compound of embodiment 23, or a pharmaceutically acceptable salt or deuterated form thereof, wherein n is 0, n′ is 1 and n″ is 2.
31. The compound of embodiment 23, or a pharmaceutically acceptable salt or deuterated form thereof, wherein n is 0, n′ is 2 and n″ is 1.
32. The compound of embodiment 23, or a pharmaceutically acceptable salt or deuterated form thereof, wherein n is 2, n′ is 1 and n″ is 0.
33. The compound of embodiment 23, or a pharmaceutically acceptable salt or deuterated form thereof, wherein n is 2, n′ is 0 and n″ is 1.
34. The compound of embodiment 24, or a pharmaceutically acceptable salt or deuterated form thereof, wherein n is 2, n′ is 0 and n″ is 0.
35. The compound of embodiment 24, or a pharmaceutically acceptable salt or deuterated form thereof, wherein n is 0, n′ is 2 and n″ is 0.
36. The compound of embodiment 24, or a pharmaceutically acceptable salt or deuterated form thereof, wherein n is 0, n′ is 0 and n″ is 2.
37. The compound of embodiment 24, or a pharmaceutically acceptable salt or deuterated form thereof, wherein n is 1, n′ is 1 and n″ is 0.
38. The compound of embodiment 24, or a pharmaceutically acceptable salt or deuterated form thereof, wherein n is 1, n′ is 0 and n″ is 1.
39. The compound of embodiment 24, or a pharmaceutically acceptable salt or deuterated form thereof, wherein n is 0, n′ is 1 and n″ is 1.
40. The compound of embodiment 25, or a pharmaceutically acceptable salt or deuterated form thereof, wherein n is 0, n′ is 1 and n″ is 0.
41. The compound of embodiment 25, or a pharmaceutically acceptable salt or deuterated form thereof, wherein n is 0, n′ is 0 and n″ is 1.
42. The compound of embodiment 25, or a pharmaceutically acceptable salt or deuterated form thereof, wherein n is 1, n′ is 0s and n″ is 0.
43. The compound of any one of embodiments 1-42, or a pharmaceutically acceptable salt or deuterated form thereof, wherein,
R² is

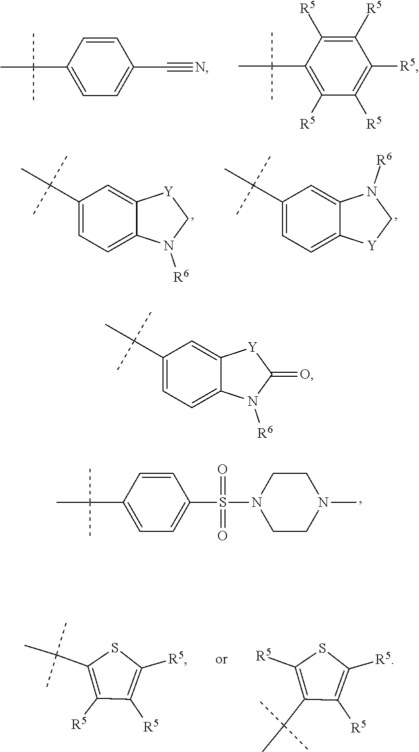

44. The compound of any one of embodiments 1-42, or a pharmaceutically acceptable salt or deuterated form thereof, wherein R² is

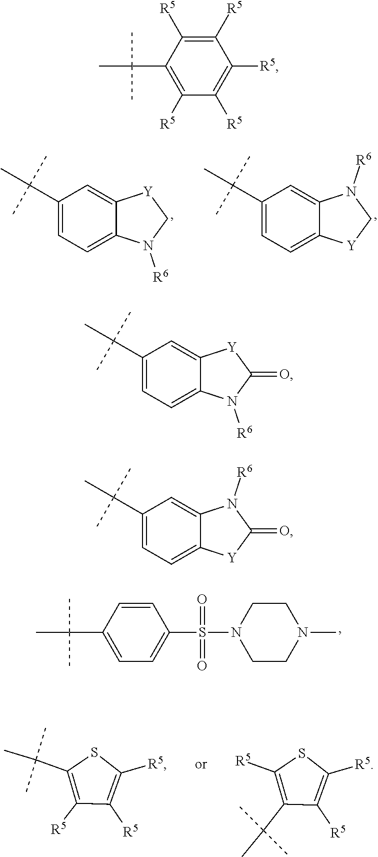

45. The compound of any one of embodiments 1-42, or a pharmaceutically acceptable salt or deuterated form thereof, wherein R² is

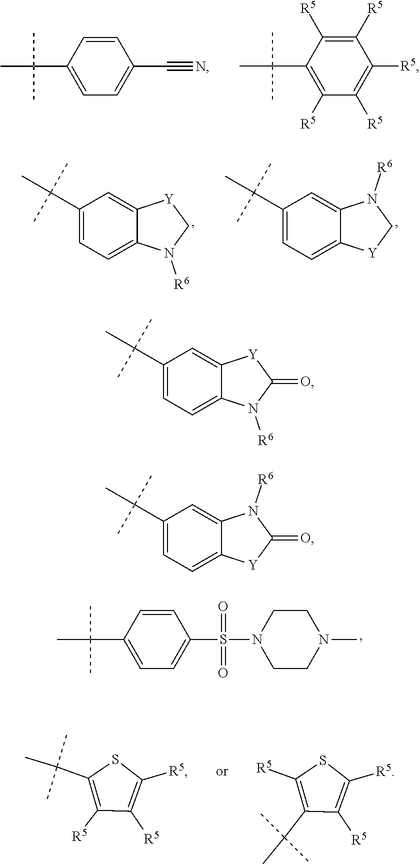

46. The compound of any one of embodiments 1-42, or a pharmaceutically acceptable salt or deuterated form thereof, wherein R² is

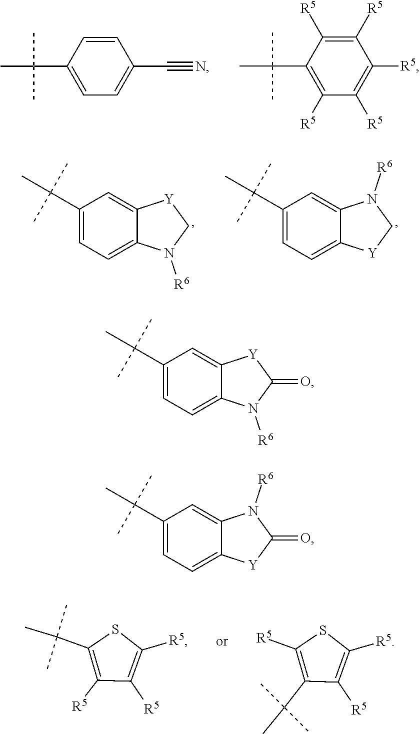

47. The compound of any one of embodiments 1-42, or a pharmaceutically acceptable salt or deuterated form thereof, wherein R² is

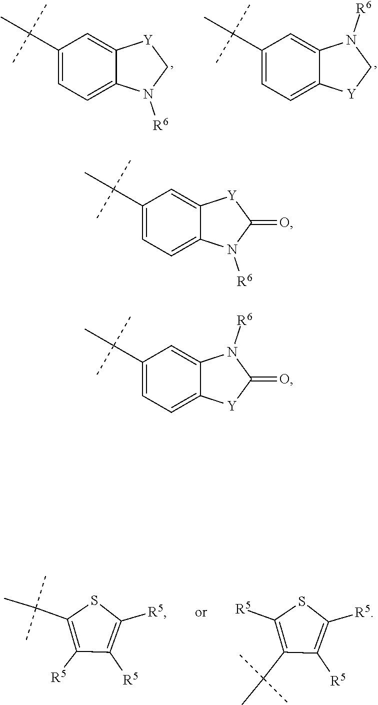

48. The compound of any one of embodiments 1-42, or a pharmaceutically acceptable salt or deuterated form thereof, wherein R² is

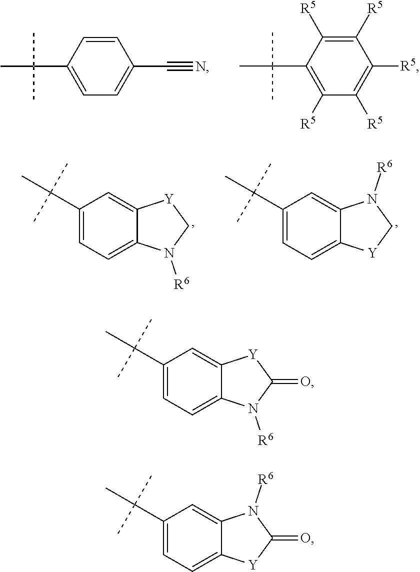

wherein Y is O, CHR⁶ or NR⁶.
49. The compound of embodiment 48, wherein R⁶ is H or CH₃.
50. The compound of any one of embodiments 1-42, or a pharmaceutically acceptable salt or deuterated form thereof, wherein R² is

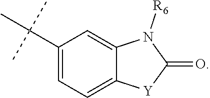

51. The compound of embodiment 50, or a pharmaceutically acceptable salt or deuterated form thereof, wherein Y is O, CHR⁶ or NR⁶.
52. The compound of any one of embodiments 1-42, or a pharmaceutically acceptable salt or deuterated form thereof, wherein R² is

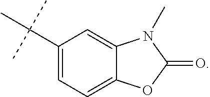

53. The compound of any one of embodiments 1-42, or a pharmaceutically acceptable salt or deuterated form thereof, wherein R² is

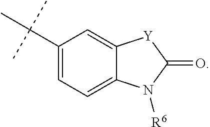

54. The compound of any one of embodiments 1-42, or a pharmaceutically acceptable salt or deuterated form thereof, wherein R² is

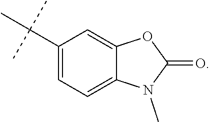

55. The compound of any one of embodiments 1-42, or a pharmaceutically acceptable salt or deuterated form thereof, wherein R² is

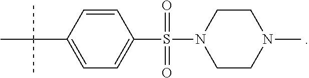

56. The compound of any one of embodiments 1-42, or a pharmaceutically acceptable salt thereof, wherein R² is

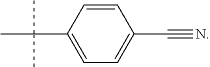

57. The compound of any one of embodiments 1-42, or a pharmaceutically acceptable salt thereof, wherein R² is

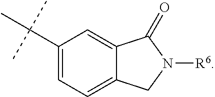

58. The compound of any one of embodiments 1-57, or a pharmaceutically acceptable salt or deuterated form thereof, wherein
L is

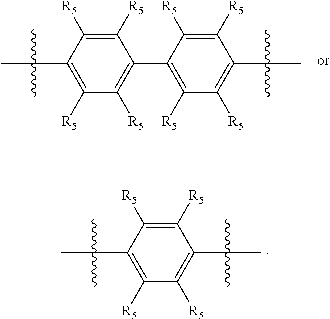

59. The compound of embodiment 58, wherein each occurrence of R⁵ is H.
60 The compound of any one of embodiments 1-57, or a pharmaceutically acceptable salt or deuterated form thereof, wherein
L is

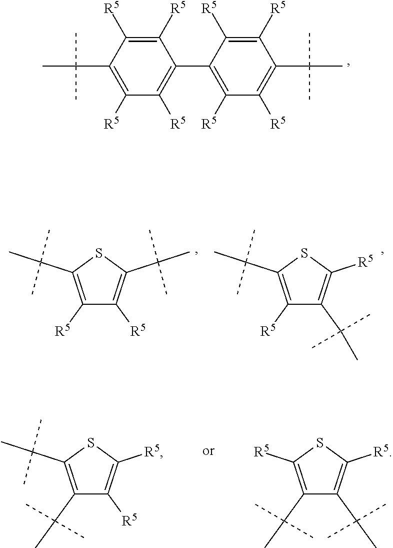

61. The compound of any one of embodiments 1-57, or a pharmaceutically acceptable salt or deuterated form thereof, wherein
L is

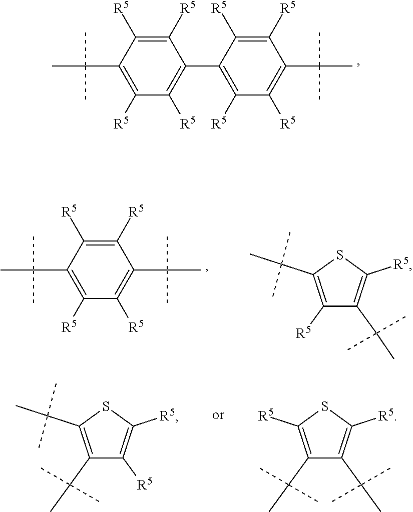

62. The compound of any one of embodiments 1-57, or a pharmaceutically acceptable salt or deuterated form thereof, wherein
L is

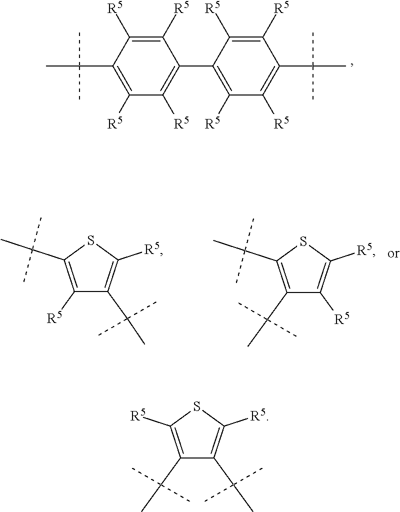

63. The compound of any one of embodiments 1-20, 22-48 and 53-55, or a pharmaceutically acceptable salt or deuterated form thereof, wherein L is

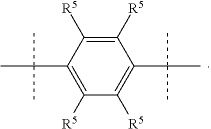

64. The compound of any one of embodiments 1-20, 22-48 and 53-55, or a pharmaceutically acceptable salt or deuterated form thereof, wherein L is

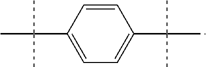

65. The compound of any one of embodiments 1-57, or a pharmaceutically acceptable salt or deuterated form thereof, wherein L is

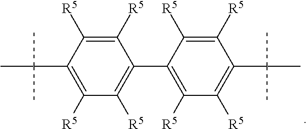

66. The compound of any one of embodiments 1-57, or a pharmaceutically acceptable salt or deuterated form thereof, wherein L is

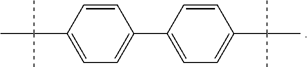

67. The compound of embodiment 1, selected from one of the compounds of Table 3.
68. The compound of any one of embodiments 1-67, wherein the compound is:

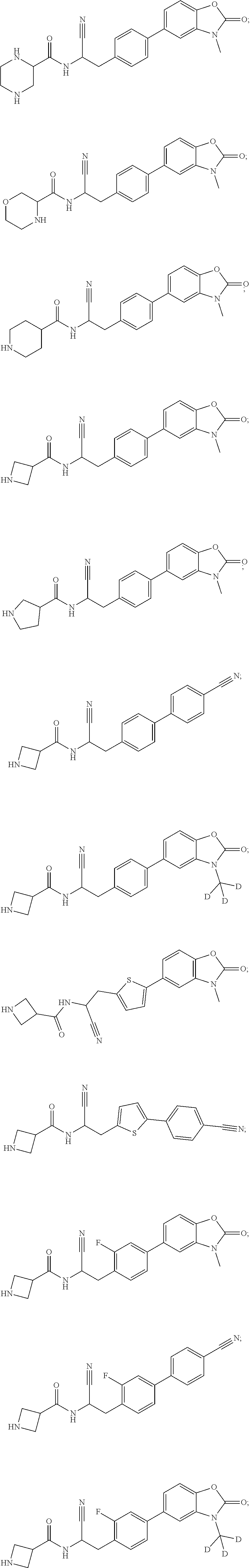

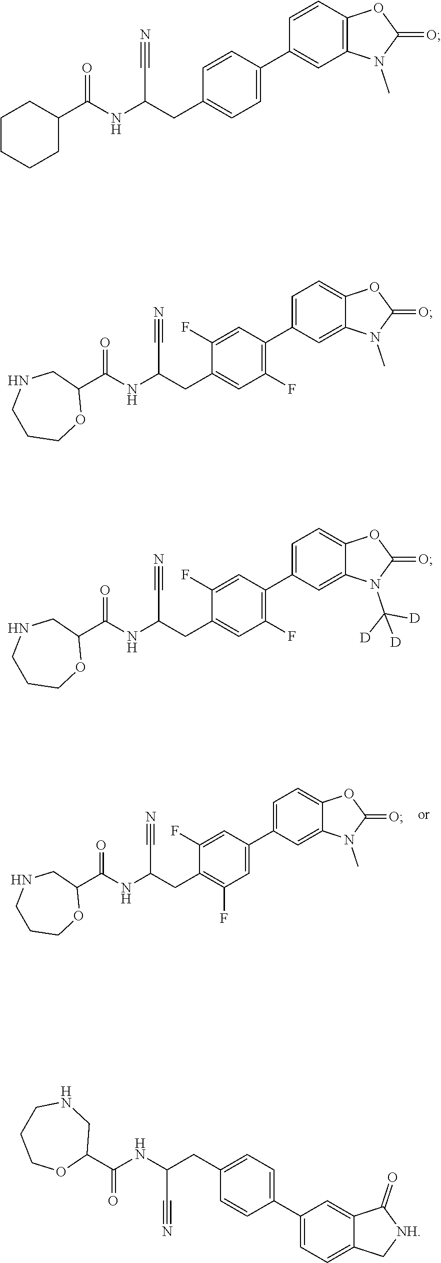 [SMILES: Cn1c(=O)oc2ccc(-c3ccc(CC(C#N)NC(=O)C4CCCCC4)cc3)cc21 ; Cn1c(=O)oc2ccc(-c3cc(F)c(CC(C#N)NC(=O)C4CNCCCO4)cc3F)cc21 ; [2H]C([2H])([2H])n1c(=O)oc2ccc(-c3cc(F)c(CC(C#N)NC(=O)C4CNCCCO4)cc3F)cc21 ; Cn1c(=O)oc2ccc(-c3cc(F)c(CC(C#N)NC(=O)C4CNCCCO4)c(F)c3)cc21 ; N#CC(Cc1ccc(-c2ccc3c(c2)C(=O)NC3)cc1)NC(=O)C1CNCCCO1]

69. The compound of any one of embodiments 1-67, wherein the compound is:

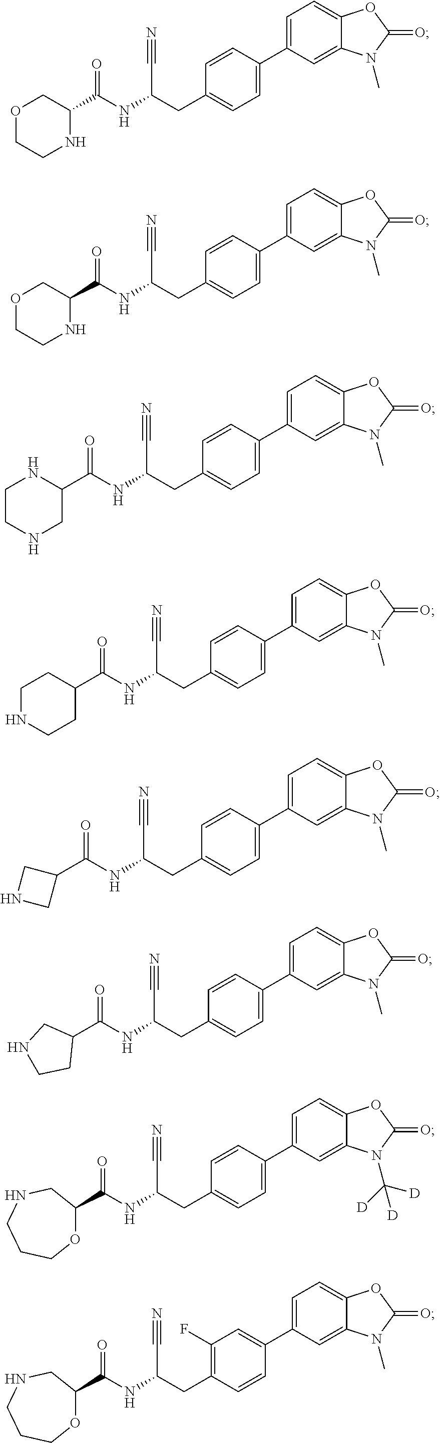

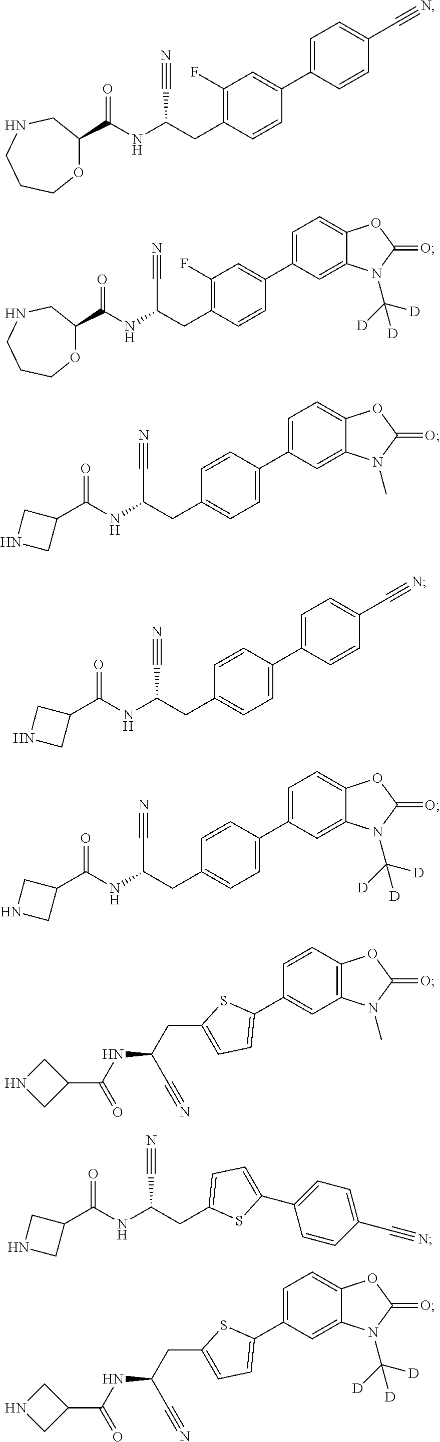

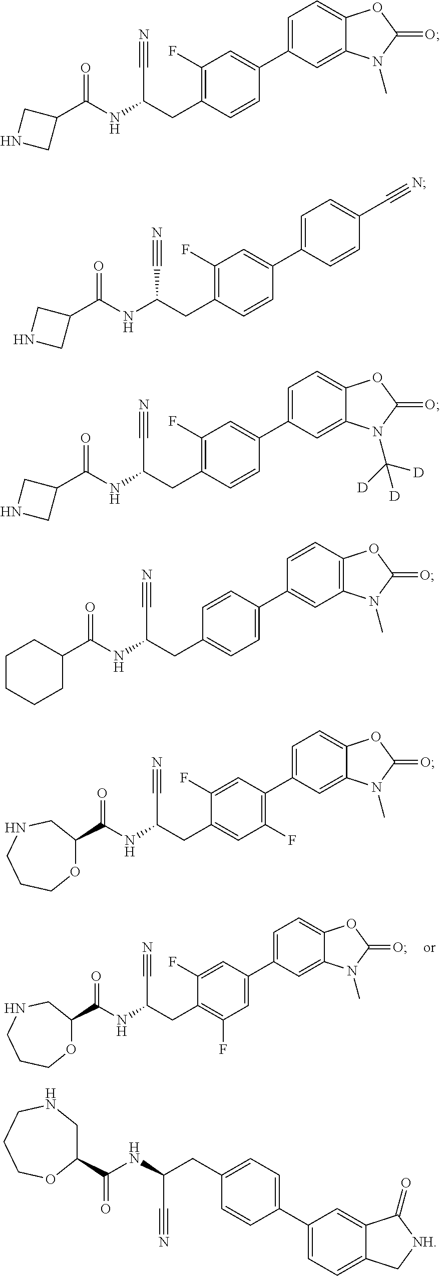

70. A compound of Formula (II), or a pharmaceutically acceptable salt or deuterated form thereof:

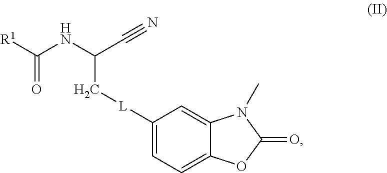

wherein,
R¹ is

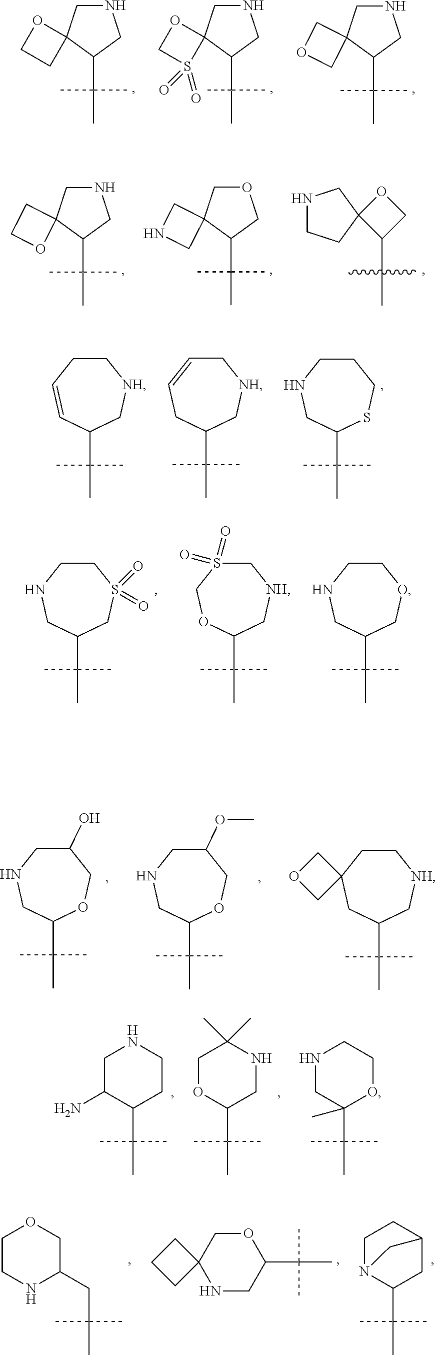

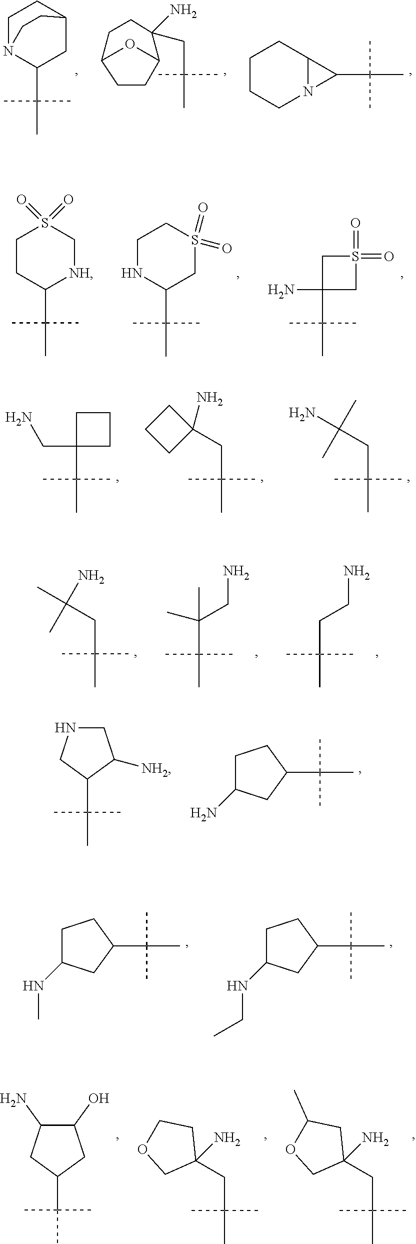

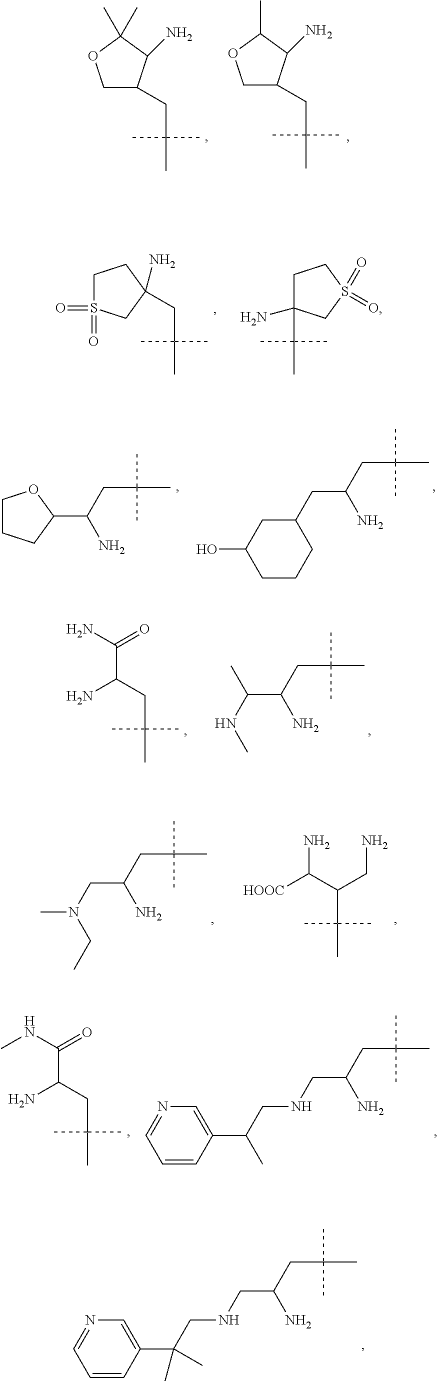

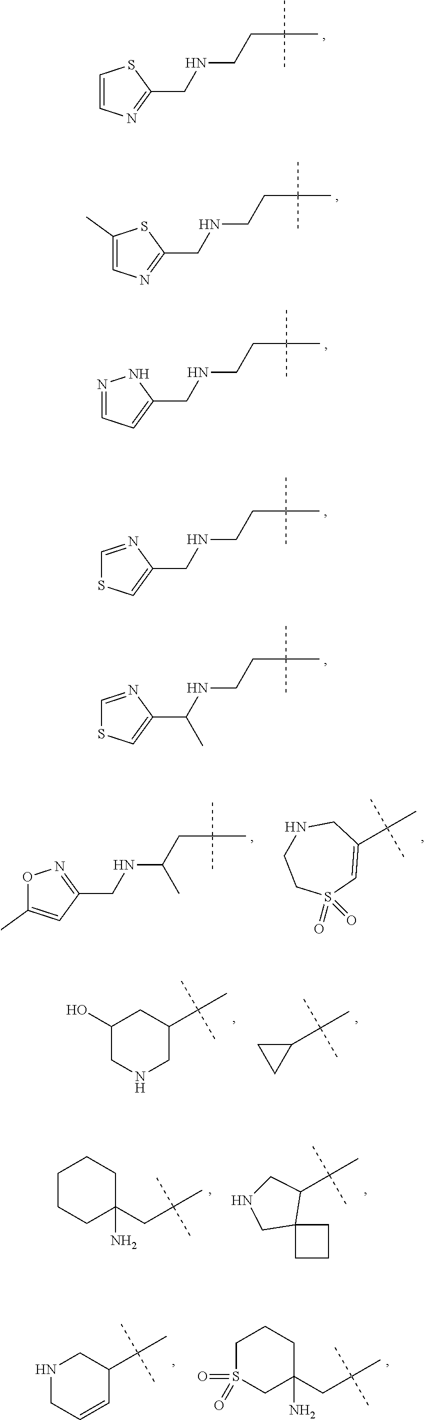

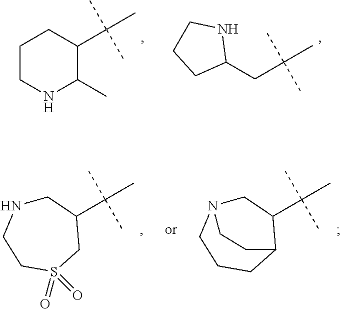

Each R² is independently H, F, Cl, Br, I or C₁-C₆ alkyl; and
L is

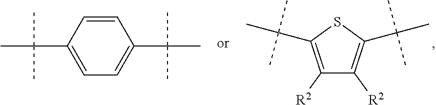

wherein the compound is not

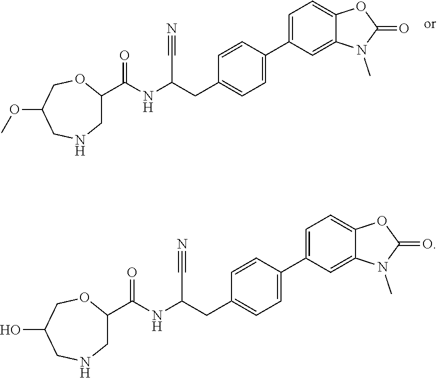 [SMILES: COC1CNCC(C(=O)NC(C#N)Cc2ccc(-c3ccc4oc(=O)n(C)c4c3)cc2)OC1 ; Cn1c(=O)oc2ccc(-c3ccc(CC(C#N)NC(=O)C4CNCC(O)CO4)cc3)cc21]

71. The compound of embodiment 70, or a pharmaceutically acceptable salt or deuterated form thereof, wherein L is

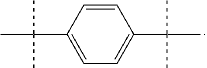

72. The compound of embodiment 70, or a pharmaceutically acceptable salt or deuterated form thereof, wherein L is

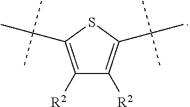

73. The compound of embodiment 70, or a pharmaceutically acceptable salt or deuterated form thereof, wherein L is

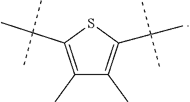

74. The compound of embodiment 70, or a pharmaceutically acceptable salt or deuterated form thereof, wherein L is

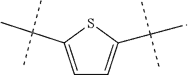

75. The compound of any one of embodiments 70-74, or a pharmaceutically acceptable salt or deuterated form thereof, wherein R¹ is

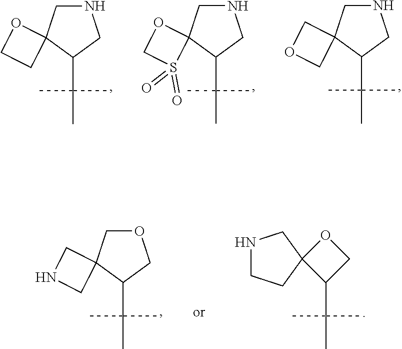

76. The compound of any one of embodiments 70-74, or a pharmaceutically acceptable salt or deuterated form thereof, wherein R¹ is

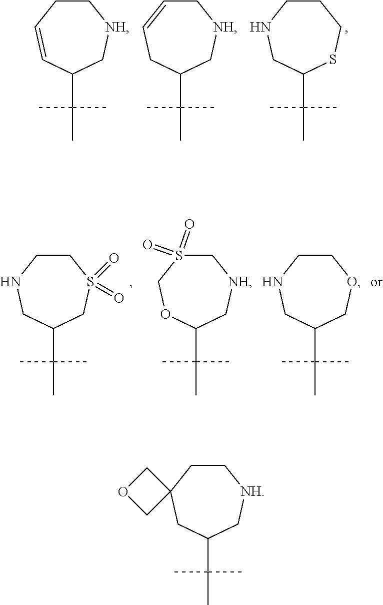

77. The compound of any one of embodiments 70-74, or a pharmaceutically acceptable salt or deuterated form thereof, wherein R¹ is

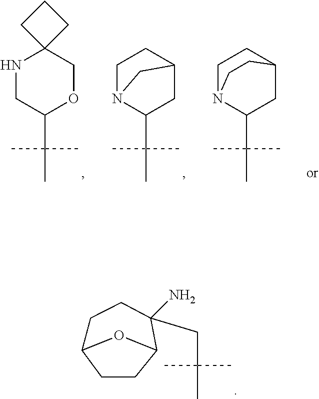

78. The compound of any one of embodiments 70-74, or a pharmaceutically acceptable salt or deuterated form thereof, wherein R¹ is

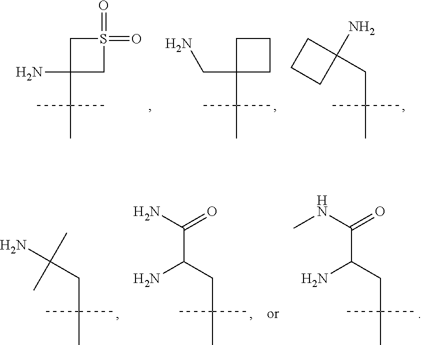

79. The compound of any one of embodiments 70-74, or a pharmaceutically acceptable salt or deuterated form thereof, wherein R¹ is

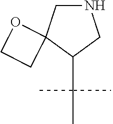

80. The compound of any one of embodiments 70-74, or a pharmaceutically acceptable salt or deuterated form thereof, wherein R¹ is

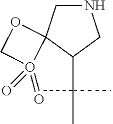

81. The compound of any one of embodiments 70-74, or a pharmaceutically acceptable salt or deuterated form thereof, wherein R¹ is

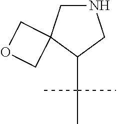

82. The compound of any one of embodiments 70-74, or a pharmaceutically acceptable salt or deuterated form thereof, wherein R¹ is

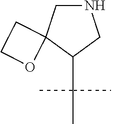

83. The compound of any one of embodiments 70-74, or a pharmaceutically acceptable salt or deuterated form thereof, wherein R¹ is

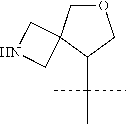

84. The compound of any one of embodiments 70-74, or a pharmaceutically acceptable salt or deuterated form thereof, wherein R¹ is

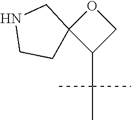

85. The compound of any one of embodiments 70-74, or a pharmaceutically acceptable salt or deuterated form thereof, wherein R¹ is

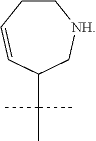

86. The compound of any one of embodiments 70-74, or a pharmaceutically acceptable salt or deuterated form thereof, wherein R¹ is

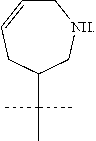

87. The compound of any one of embodiments 70-74, or a pharmaceutically acceptable salt or deuterated form thereof, wherein R¹ is

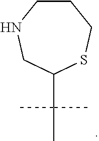

88. The compound of any one of embodiments 70-74, or a pharmaceutically acceptable salt or deuterated form thereof, wherein R¹ is

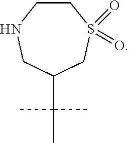

89. The compound of any one of embodiments 70-74, or a pharmaceutically acceptable salt or deuterated form thereof, wherein R¹ is

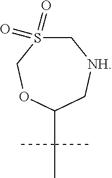

90. The compound of any one of embodiments 70-74, or a pharmaceutically acceptable salt thereof, wherein R¹ is

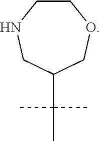

91. The compound of any one of embodiments 70-74, or a pharmaceutically acceptable salt or deuterated form thereof, wherein R¹ is

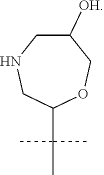

92. The compound of any one of embodiments 70-74, or a pharmaceutically acceptable salt or deuterated form thereof, wherein R¹ is

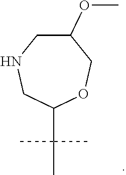

93. The compound of any one of embodiments 70-74, or a pharmaceutically acceptable salt or deuterated form thereof, wherein R¹ is

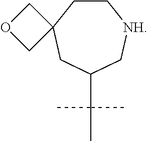

94. The compound of any one of embodiments 70-74, or a pharmaceutically acceptable salt or deuterated form thereof, wherein R¹ is

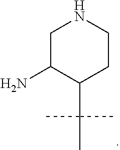

95. The compound of any one of embodiments 70-74, or a pharmaceutically acceptable salt or deuterated form thereof, wherein R¹ is

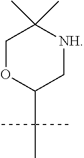

96. The compound of any one of embodiments 70-74, or a pharmaceutically acceptable salt or deuterated form thereof, wherein R¹ is

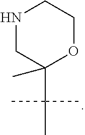

97. The compound of any one of embodiments 70-74, or a pharmaceutically acceptable salt or deuterated form thereof, wherein R¹ is

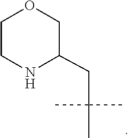

98. The compound of any one of embodiments 70-74, or a pharmaceutically acceptable salt or deuterated form thereof, wherein R¹ is

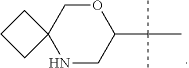

99. The compound of any one of embodiments 70-74, or a pharmaceutically acceptable salt or deuterated form thereof, wherein R¹ is

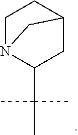

100. The compound of any one of embodiments 70-74, or a pharmaceutically acceptable salt or deuterated form thereof, wherein R¹ is

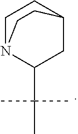

101. The compound of any one of embodiments 70-74, or a pharmaceutically acceptable salt or deuterated form thereof, wherein R¹ is

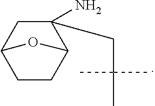

102. The compound of any one of embodiments 70-74, or a pharmaceutically acceptable salt or deuterated form thereof, wherein R¹ is

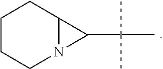

103. The compound of any one of embodiments 70-74, or a pharmaceutically acceptable salt or deuterated form thereof, wherein R¹ is

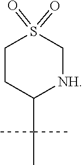

104. The compound of any one of embodiments 70-74, or a pharmaceutically acceptable salt or deuterated form thereof, wherein R¹ is

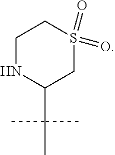

105. The compound of any one of embodiments 70-74, or a pharmaceutically acceptable salt or deuterated form thereof, wherein R¹ is

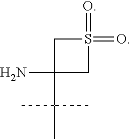

106. The compound of any one of embodiments 70-74, or a pharmaceutically acceptable salt or deuterated form thereof, wherein R¹ is

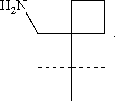

107. The compound of any one of embodiments 70-74, or a pharmaceutically acceptable salt or deuterated form thereof, wherein R¹ is

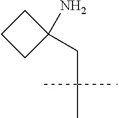

108. The compound of any one of embodiments 70-74, or a pharmaceutically acceptable salt or deuterated form thereof, wherein R¹ is

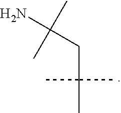

109. The compound of any one of embodiments 70-74, or a pharmaceutically acceptable salt or deuterated form thereof, wherein R¹ is

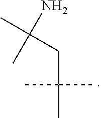

110. The compound of any one of embodiments 70-74, or a pharmaceutically acceptable salt or deuterated form thereof, wherein R¹ is

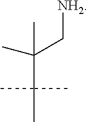

111. The compound of any one of embodiments 70-74, or a pharmaceutically acceptable salt or deuterated form thereof, wherein R¹ is

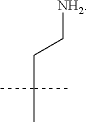

112. The compound of any one of embodiments 70-74, or a pharmaceutically acceptable salt or deuterated form thereof, wherein R¹ is

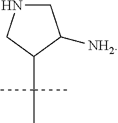

113. The compound of any one of embodiments 70-74, or a pharmaceutically acceptable salt or deuterated form thereof, wherein R¹ is

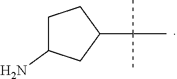

114. The compound of any one of embodiments 70-74, or a pharmaceutically acceptable salt or deuterated form thereof, wherein R¹ is

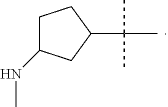

115. The compound of any one of embodiments 70-74, or a pharmaceutically acceptable salt or deuterated form thereof, wherein R¹ is

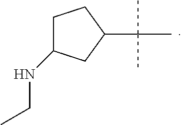

116. The compound of any one of embodiments 70-74, or a pharmaceutically acceptable salt or deuterated form thereof, wherein R¹ is

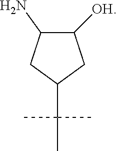

117. The compound of any one of embodiments 70-74, or a pharmaceutically acceptable salt or deuterated form thereof, wherein R¹ is

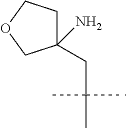

118. The compound of any one of embodiments 70-74, or a pharmaceutically acceptable salt or deuterated form thereof, wherein R¹ is

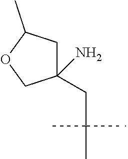

119. The compound of any one of embodiments 70-74, or a pharmaceutically acceptable salt or deuterated form thereof, wherein R¹ is

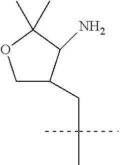

120. The compound of any one of embodiments 70-74, or a pharmaceutically acceptable salt or deuterated form thereof, wherein R¹ is

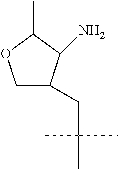

121. The compound of any one of embodiments 70-74, or a pharmaceutically acceptable salt or deuterated form thereof, wherein R¹ is

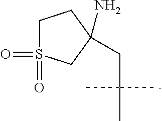

122. The compound of any one of embodiments 70-74, or a pharmaceutically acceptable salt or deuterated form thereof, wherein R¹ is

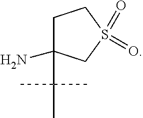

123. The compound of any one of embodiments 70-74, or a pharmaceutically acceptable salt or deuterated form thereof, wherein R¹ is

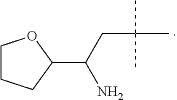

124. The compound of any one of embodiments 70-74, or a pharmaceutically acceptable salt or deuterated form thereof, wherein R¹ is

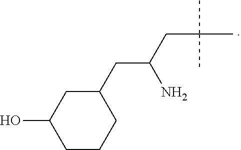

125. The compound of any one of embodiments 70-74, or a pharmaceutically acceptable salt or deuterated form thereof, wherein R¹ is

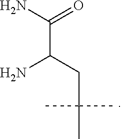

126. The compound of any one of embodiments 70-74, or a pharmaceutically acceptable salt or deuterated form thereof, wherein R¹ is

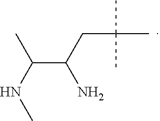

127. The compound of any one of embodiments 70-74, or a pharmaceutically acceptable salt or deuterated form thereof, wherein R¹ is

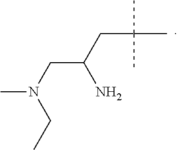

128. The compound of any one of embodiments 70-74, or a pharmaceutically acceptable salt or deuterated form thereof, wherein R¹ is

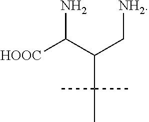

129. The compound of any one of embodiments 70-74, or a pharmaceutically acceptable salt or deuterated form thereof, wherein R¹ is

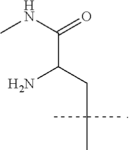

130. The compound of any one of embodiments 70-74, or a pharmaceutically acceptable salt or deuterated form thereof, wherein R¹ is

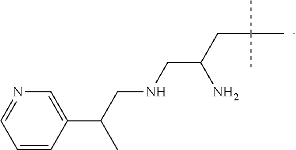

131. The compound of any one of embodiments 70-74, or a pharmaceutically acceptable salt or deuterated form thereof, wherein R¹ is

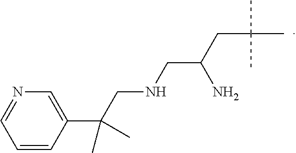

132. The compound of any one of embodiments 70-74, or a pharmaceutically acceptable salt or deuterated form thereof, wherein R¹ is

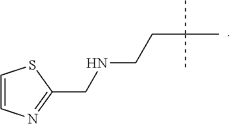

133. The compound of any one of embodiments 70-74, or a pharmaceutically acceptable salt or deuterated form thereof, wherein R¹ is

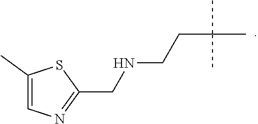

134. The compound of any one of embodiments 70-74, or a pharmaceutically acceptable salt or deuterated form thereof, wherein R¹ is

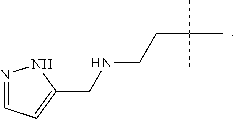

135. The compound of any one of embodiments 70-74, or a pharmaceutically acceptable salt or deuterated form thereof, wherein R¹ is

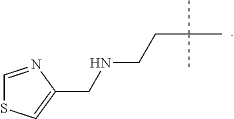

136. The compound of any one of embodiments 70-74, or a pharmaceutically acceptable salt or deuterated form thereof, wherein R¹ is

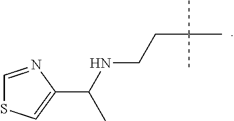

137. The compound of any one of embodiments 70-74, or a pharmaceutically acceptable salt or deuterated form thereof, wherein R¹ is

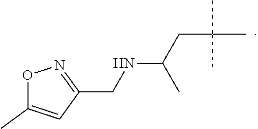

138. The compound of any one of embodiments 70-74, or a pharmaceutically acceptable salt or deuterated form thereof, wherein R¹ is

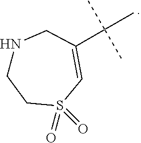

139. The compound of any one of embodiments 70-74, or a pharmaceutically acceptable salt or deuterated form thereof, wherein R¹ is

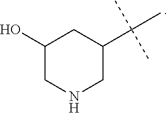

140. The compound of any one of embodiments 70-74, or a pharmaceutically acceptable salt or deuterated form thereof, wherein R¹ is

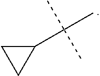

141. The compound of any one of embodiments 70-74, or a pharmaceutically acceptable salt or deuterated form thereof, wherein R¹ is

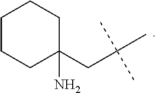

142. The compound of any one of embodiments 70-74, or a pharmaceutically acceptable salt or deuterated form thereof, wherein R¹ is

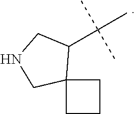

143. The compound of any one of embodiments 70-74, or a pharmaceutically acceptable salt or deuterated form thereof, wherein R¹ is

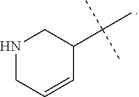

144. The compound of any one of embodiments 70-74, or a pharmaceutically acceptable salt or deuterated form thereof, wherein R¹ is

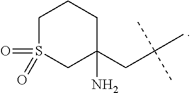

145. The compound of any one of embodiments 70-74, or a pharmaceutically acceptable salt or deuterated form thereof, wherein R¹ is

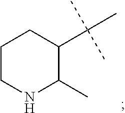

146. The compound of any one of embodiments 70-74, or a pharmaceutically acceptable salt or deuterated form thereof, wherein R¹ is

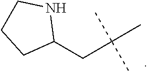

147. The compound of any one of embodiments 70-74, or a pharmaceutically acceptable salt or deuterated form thereof, wherein R¹ is

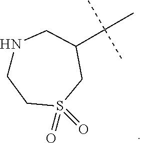

148. The compound of any one of embodiments 70-74, or a pharmaceutically acceptable salt or deuterated form thereof, wherein R¹ is

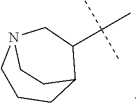

149. The compound of any one of embodiments 70-148, or a pharmaceutically accept salt or deuterated form thereof, wherein the compound is:

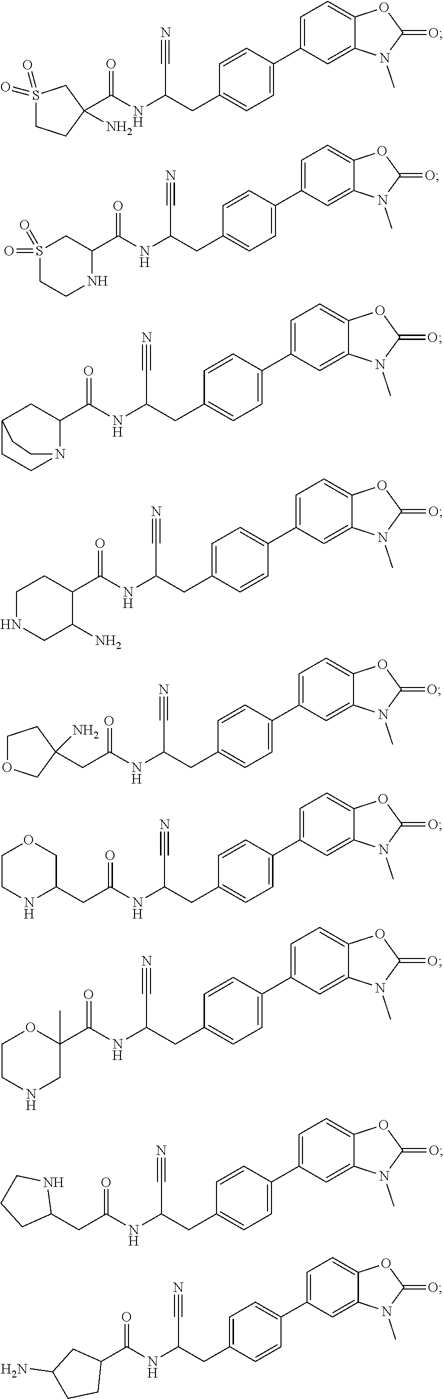

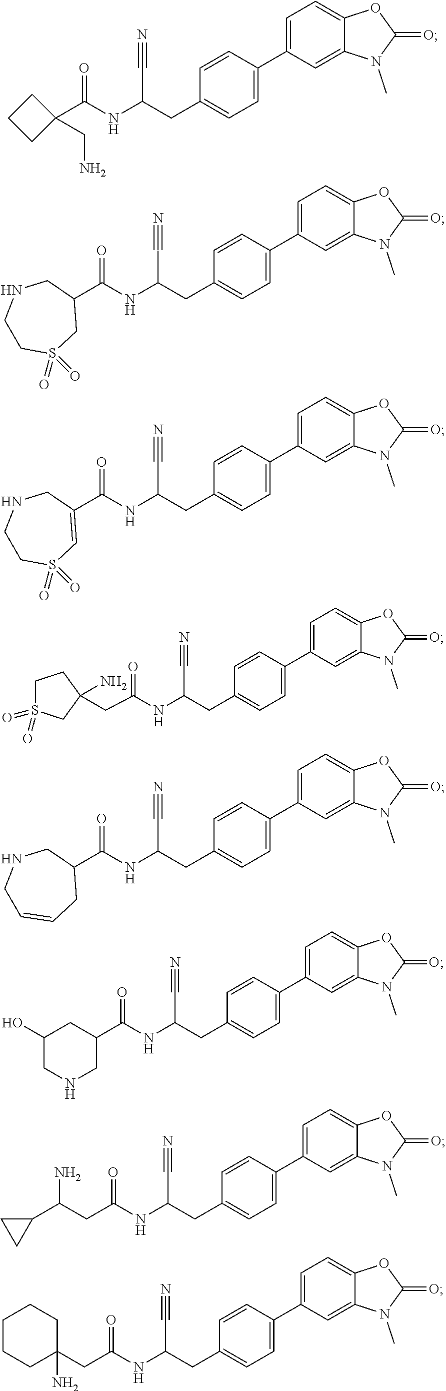

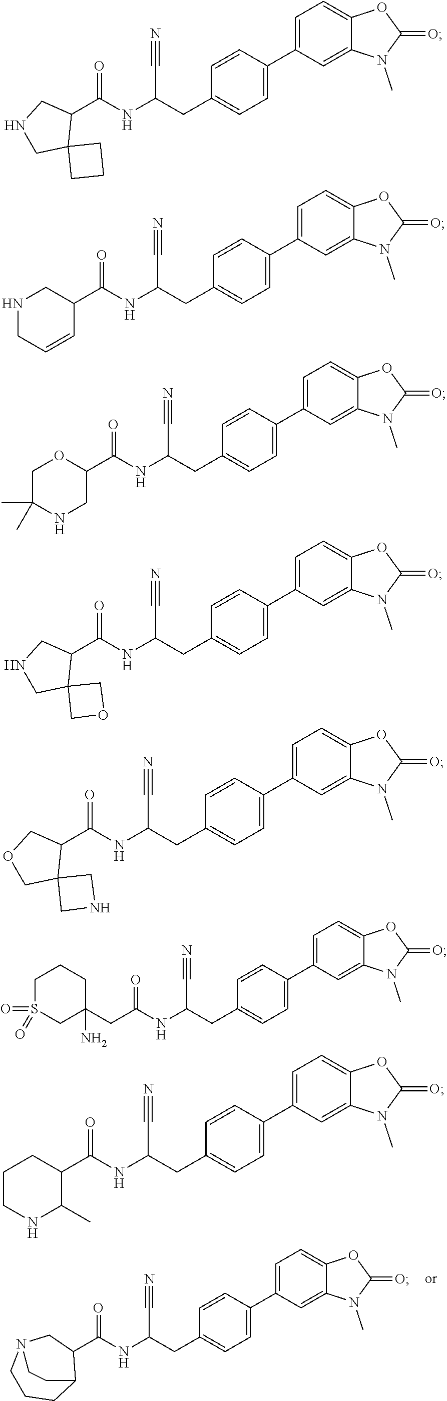

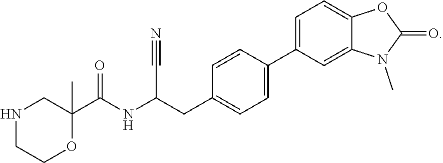 [SMILES: Cn1c(=O)oc2ccc(-c3ccc(CC(C#N)NC(=O)C4(C)CNCCO4)cc3)cc21]

150. The compound of any one of embodiments 70-148, or a pharmaceutically accept salt or deuterated form thereof, wherein the compound is:

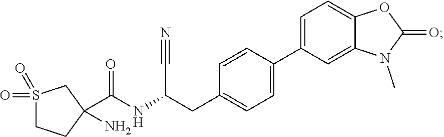 [SMILES: Cn1c(=O)oc2ccc(-c3ccc(C[C@@H](C#N)NC(=O)C4(N)CCS(=O)(=O)C4)cc3)cc21]

151. A compound of Formula (III), or a pharmaceutically acceptable salt or deuterated form thereof:

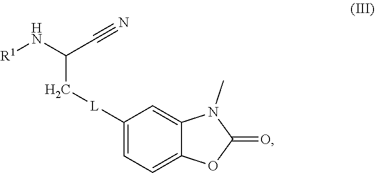

wherein,
R¹ is

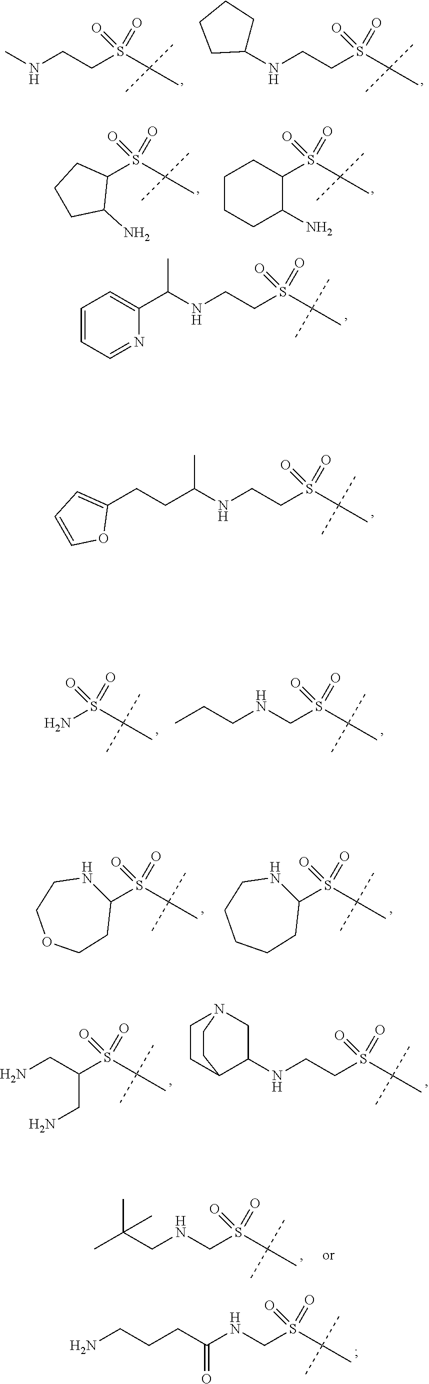

each R² is independently H, F, Cl, Br, I or C₁-C₆ alkyl; and
L is

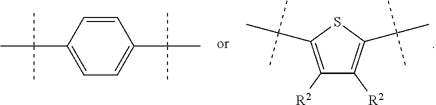

152. The compound of embodiment 151, or a pharmaceutically acceptable salt or deuterated form thereof, wherein L is

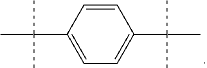

153. The compound of embodiment 151, or a pharmaceutically acceptable salt or deuterated form thereof, wherein L is

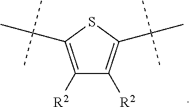

154. The compound of embodiment 153, or a pharmaceutically acceptable salt or deuterated form thereof, wherein L is

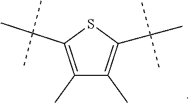

155. The compound of embodiment 153, or a pharmaceutically acceptable salt or deuterated form thereof, wherein L is

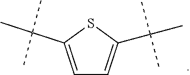

156. The compound of any one of embodiments 151-155, or a pharmaceutically acceptable salt or deuterated form thereof, wherein R¹ is

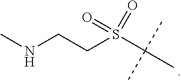

157. The compound of any one of embodiments 151-155, or a pharmaceutically acceptable salt or deuterated form thereof, wherein R¹ is

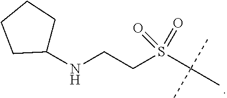

158. The compound of any one of embodiments 151-155, or a pharmaceutically acceptable salt or deuterated form thereof, wherein R¹ is

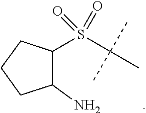

159. The compound of any one of embodiments 151-155, or a pharmaceutically acceptable salt or deuterated form thereof, wherein R¹ is

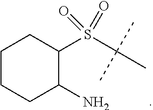

160. The compound of any one of embodiments 151-155, or a pharmaceutically acceptable salt or deuterated form thereof, wherein R¹ is

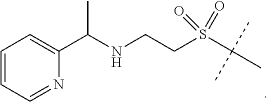

161. The compound of any one of embodiments 151-155, or a pharmaceutically acceptable salt or deuterated form thereof, wherein R¹ is

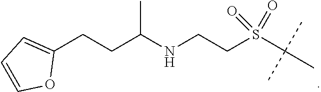

162. The compound of any one of embodiments 151-155, or a pharmaceutically acceptable salt or deuterated form thereof, wherein R¹ is

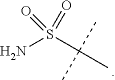

163. The compound of any one of embodiments 151-155, or a pharmaceutically acceptable salt or deuterated form thereof, wherein R¹ is

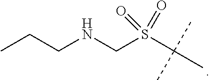

164. The compound of any one of embodiments 151-155, or a pharmaceutically acceptable salt or deuterated form thereof, wherein R¹ is

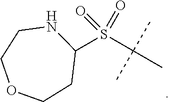

165. The compound of any one of embodiments 151-155, or a pharmaceutically acceptable salt or deuterated form thereof, wherein R¹ is

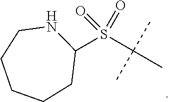

166. The compound of any one of embodiments 151-155, or a pharmaceutically acceptable salt or deuterated form thereof, wherein R¹ is

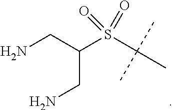

167. The compound of any one of embodiments 151-155, or a pharmaceutically acceptable salt or deuterated form thereof, wherein R¹ is

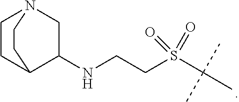

168. The compound of any one of embodiments 151-155, or a pharmaceutically acceptable salt or deuterated form thereof, wherein R¹ is

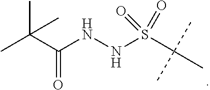

169. The compound of any one of embodiments 151-155, or a pharmaceutically acceptable salt or deuterated form thereof, wherein R¹ is

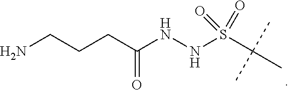

170. The compound of any one of embodiments 151-169, or a pharmaceutically acceptable salt or deuterated form thereof, wherein the compound is:

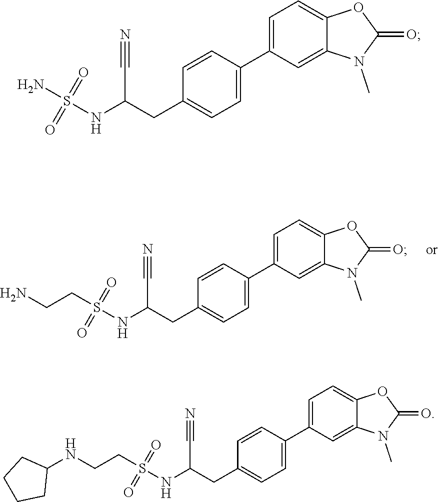

171. The compound of any one of embodiments 151-169, or a pharmaceutically acceptable salt or deuterated form thereof, wherein the compound is:

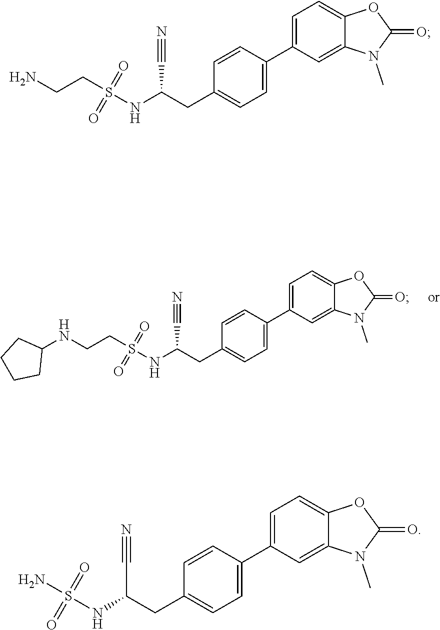

172. A compound of Formula (I-I):

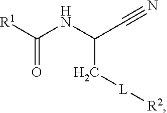

or a pharmaceutically acceptable salt or deuterated form thereof, wherein,
R¹ is

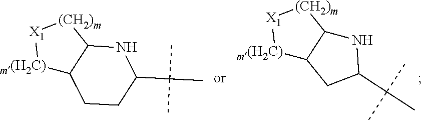

R² is

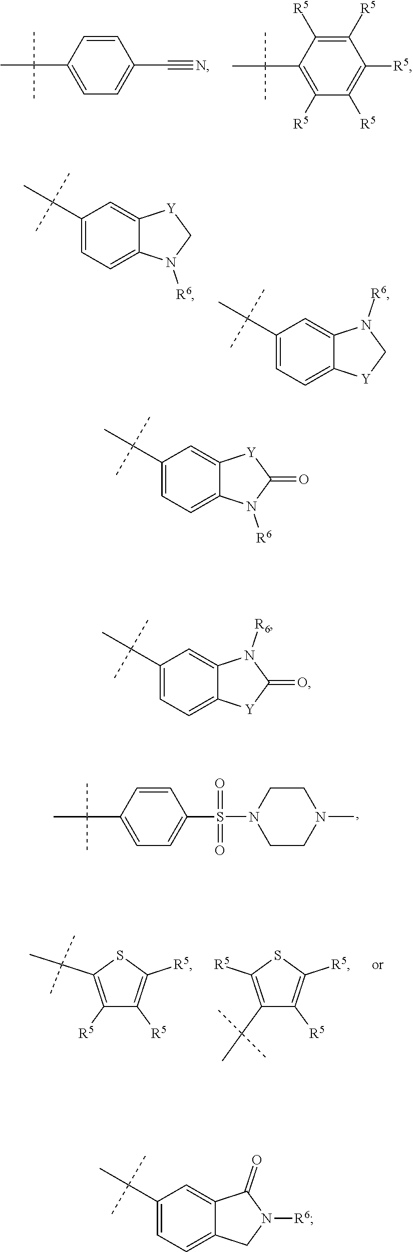

X₁, X₂ and X₃ are independently O, S, NR³ or CR³R⁴;
each R³, R⁴ and R⁵ is independently H, F, Cl, Br, I or C₁-C₆ alkyl;
each R⁶ is independently H or C₁-C₆ alkyl;
each Y is independently O, S, CHR⁶ or NR⁶;
m and m′ are each independently an integer from 0-3, and the total sum of m and m′ is ≤3; and

L is

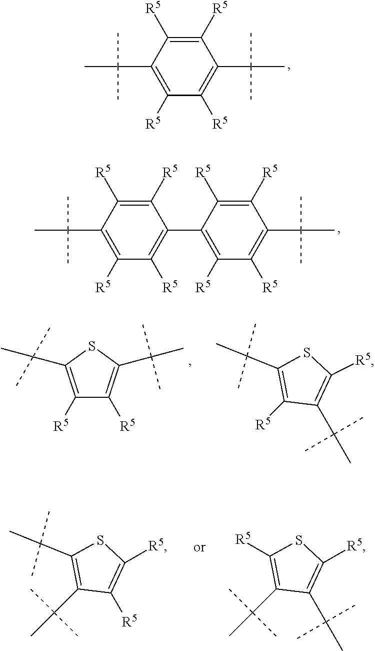

provided that, the compound is not:

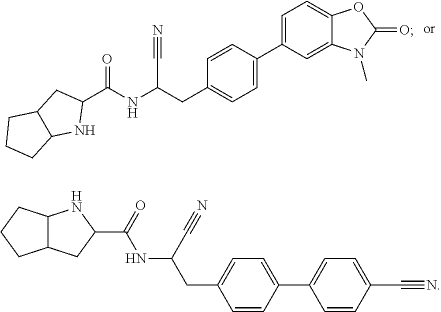 [SMILES: Cn1c(=O)oc2ccc(-c3ccc(CC(C#N)NC(=O)C4CC5CCCC5N4)cc3)cc21 ; "or" ; N#Cc1ccc(-c2ccc(CC(C#N)NC(=O)C3CC4CCCC4N3)cc2)cc1]

173. The compound of embodiment 172, or a pharmaceutically acceptable salt or deuterated form thereof, having the following chemical structure:

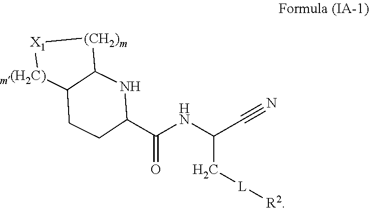

174. The compound of embodiment 172, or a pharmaceutically acceptable salt or deuterated form thereof, having the following chemical structure:

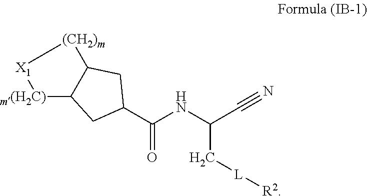

175. The compound of embodiment 172, or a pharmaceutically acceptable salt or deuterated form thereof, having the following chemical structure:

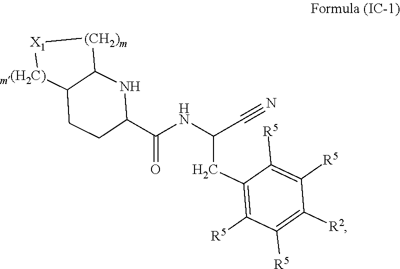

wherein at least one R₅ is F.
176. The compound of embodiment 172, or a pharmaceutically acceptable salt or deuterated form thereof, having the following chemical structure:

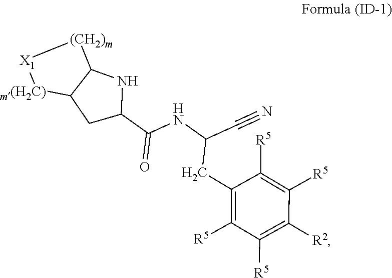

wherein at least one R₅ is F.
177. The compound of embodiment 172, or a pharmaceutically acceptable salt or deuterated form thereof, wherein,
R¹ is

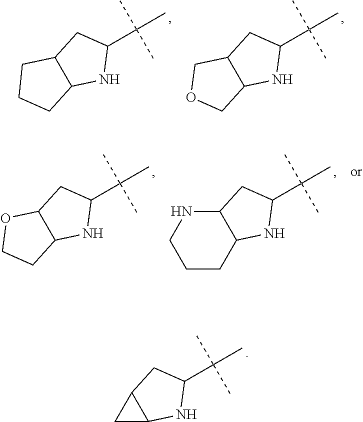

178. The compound of embodiment 177, or a pharmaceutically acceptable salt or deuterated form thereof, wherein, R¹ is

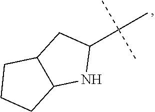

179. The compound of embodiment 177, or a pharmaceutically acceptable salt or deuterated form thereof, wherein, R¹ is

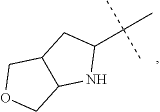

180. The compound of embodiment 177, or a pharmaceutically acceptable salt or deuterated form thereof, wherein, R¹ is

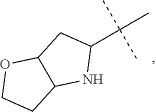

181. The compound of embodiment 177, or a pharmaceutically acceptable salt or deuterated form thereof, wherein, R¹ is

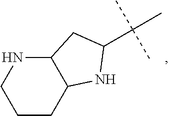

182. The compound of embodiment 177, or a pharmaceutically acceptable salt or deuterated form thereof, wherein, R¹ is

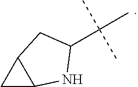

183. The compound of any one of embodiments 176-178, or a pharmaceutically acceptable salt or deuterated form thereof, wherein R¹ is

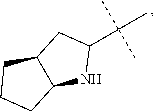

184. The compound of any one of embodiments 176-178, or a pharmaceutically acceptable salt or deuterated form thereof, wherein R¹ is

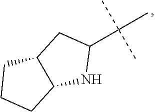

185. The compound of any one of embodiments 176-178, or a pharmaceutically acceptable salt or deuterated form thereof, wherein R¹ is

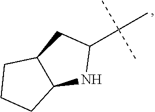

186. The compound of any one of embodiments 176-177 or 179, or a pharmaceutically acceptable salt or deuterated form thereof, wherein R¹ is

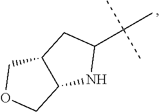

187. The compound of any one of embodiments 176-177 or 179, or a pharmaceutically acceptable salt or deuterated form thereof, wherein R¹ is

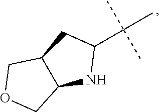

188. The compound of any one of embodiments 176-177 or 179, or a pharmaceutically acceptable salt or deuterated form thereof, wherein R¹ is

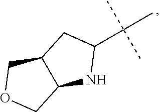

189. The compound of any one of embodiments 176-177 or 182, or a pharmaceutically acceptable salt or deuterated form thereof, wherein R¹ is

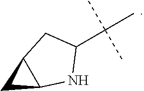

190. The compound of any one of embodiments 173-176, or a pharmaceutically acceptable salt or deuterated form thereof, wherein m is 1 and m′ is 1.
191. The compound of any one of embodiments 173-176, or a pharmaceutically acceptable salt or deuterated form thereof, wherein m is 1 and m′ is 2.
192. The compound of any one of embodiments 173-176, or a pharmaceutically acceptable salt or deuterated form thereof, wherein m is 2 and m′ is 1.
193. The compound of any one of embodiments 173-176, or a pharmaceutically acceptable salt or deuterated form thereof, wherein m is 1 and m′ is 0.
194. The compound of any one of embodiments 173-176, or a pharmaceutically acceptable salt or deuterated form thereof, wherein m is 0 and m′ is 1.
195. The compound of any one of embodiments 173-176, or a pharmaceutically acceptable salt or deuterated form thereof, wherein m is 2 and m′ is 0.
196. The compound of any one of embodiments 173-176, or a pharmaceutically acceptable salt or deuterated form thereof, wherein m is 0 and m′ is 2.
197. The compound of any one of embodiments 173-176, or a pharmaceutically acceptable salt or deuterated form thereof, wherein m is 0 and m′ is 0.
198. The compound of any one of embodiments 173-176, or a pharmaceutically acceptable salt or deuterated form thereof, wherein m is 2 and m′ is 0.
199. The compound of any one of embodiments 173-176, or a pharmaceutically acceptable salt or deuterated form thereof, wherein m is 3 and m′ is 0.
200. The compound of any one of embodiments 173-176 or 191-200, or a pharmaceutically acceptable salt or deuterated form thereof, wherein X₁ is O.
201. The compound of any one of embodiments 173-176 or 191-200, or a pharmaceutically acceptable salt or deuterated form thereof, wherein X₁ is S.
202. The compound of any one of embodiments 173-176 or 191-200, or a pharmaceutically acceptable salt or deuterated form thereof, wherein X₁ is NH.
203. The compound of any one of embodiments 173-176 or 191-200, or a pharmaceutically acceptable salt or deuterated form thereof, wherein X₁ is CH₂.
204. The compound of any one of embodiments 173-203, or a pharmaceutically acceptable salt or deuterated form thereof, wherein,
R² is

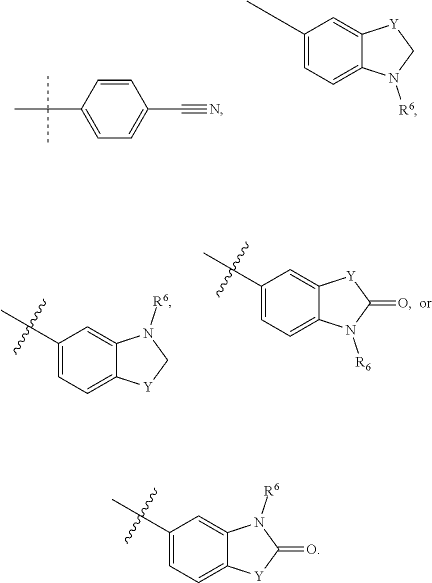

205. The compound of any one of embodiments of any one of embodiments 173-204, or a pharmaceutically acceptable salt or deuterated form thereof, wherein R² is

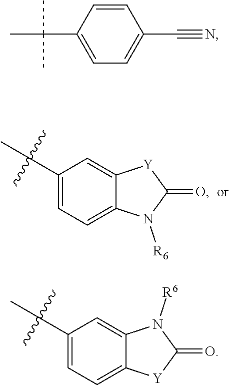

206. The compound of any one of embodiments of any one of embodiments 173-204, or a pharmaceutically acceptable salt or deuterated form thereof, wherein R² is

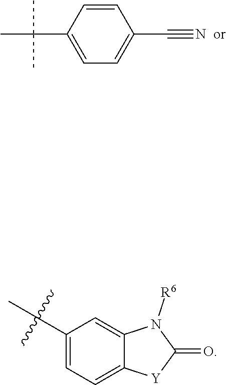

207. The compound of any one of embodiments 174-206, or a pharmaceutically acceptable salt or deuterated form thereof, wherein R² is

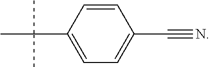

208. The compound of any one of embodiments 174-205, or a pharmaceutically acceptable salt or deuterated form thereof, wherein R² is

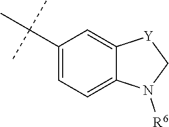

209. The compound of any one of embodiments 174-205, or a pharmaceutically acceptable salt or deuterated form thereof, wherein R² is

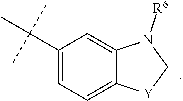

210. The compound of any one of embodiments 174-205, or a pharmaceutically acceptable salt or deuterated form thereof, wherein R² is

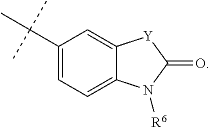

211. The compound of any one of embodiments 174-205, or a pharmaceutically acceptable salt or deuterated form thereof, wherein R² is

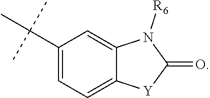

212. The compound of any one of embodiments 174-205, or a pharmaceutically acceptable salt thereof, wherein R² is

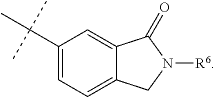

213. The compound of any one of embodiments 210-211, or a pharmaceutically acceptable salt or deuterated form thereof, wherein Y is O.
214. The compound of any one of embodiments 201-211, or a pharmaceutically acceptable salt or deuterated form thereof, wherein R₆ is —CH₃.
215. The compound of any one of embodiments 174-176 or 177-214, or a pharmaceutically acceptable salt or deuterated form thereof, wherein
L is

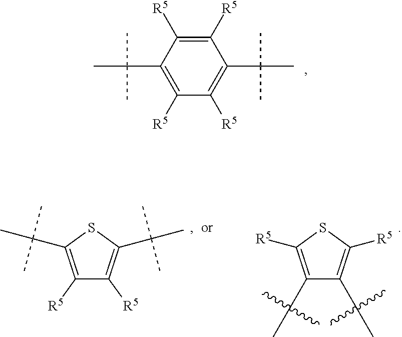

216. The compound of any one of embodiments 174-176 or 177-214, or a pharmaceutically acceptable salt or deuterated form thereof, wherein
L is

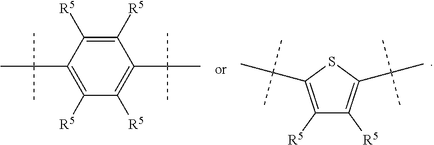

217. The compound of any one of embodiments 174-176 or 177-214, or a pharmaceutically acceptable salt or deuterated form thereof, wherein
L is

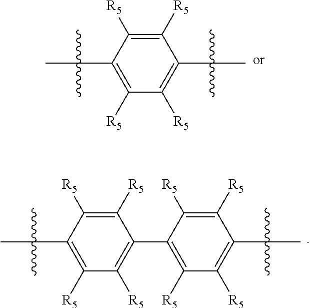

218. The compound of any one of embodiments 174-176 or 177-214, or a pharmaceutically acceptable salt or deuterated form thereof, wherein
L is

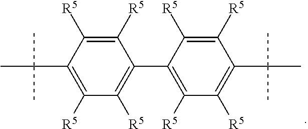

219. The compound of any one of embodiments 174-176 or 177-214, or a pharmaceutically acceptable salt or deuterated form thereof, wherein
L is

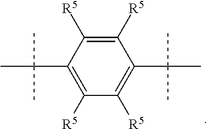

220. The compound of any one of embodiments 174-176 or 177-214, or a pharmaceutically acceptable salt or deuterated form thereof, wherein L is

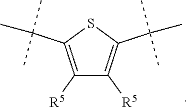

221. The compound of any one of embodiments 217-230, or a pharmaceutically acceptable salt or deuterated form thereof, wherein each R⁵ is H.
222. The compound of any one of embodiments 217-230, or a pharmaceutically acceptable salt or deuterated form thereof, wherein at least one R⁵ is F.
223. The compound of embodiment 172, or a pharmaceutically acceptable salt or deuterated form thereof selected from one of the compounds of Table 3.
224. The compound of any one of embodiments 172-223, or a pharmaceutically acceptable salt or deuterated form thereof, wherein the compound is:

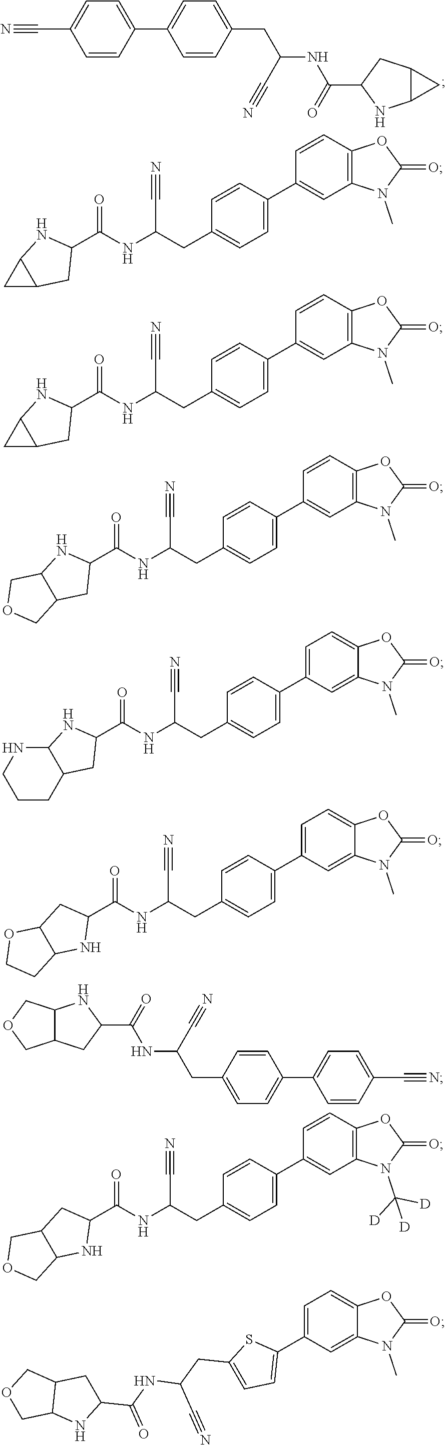

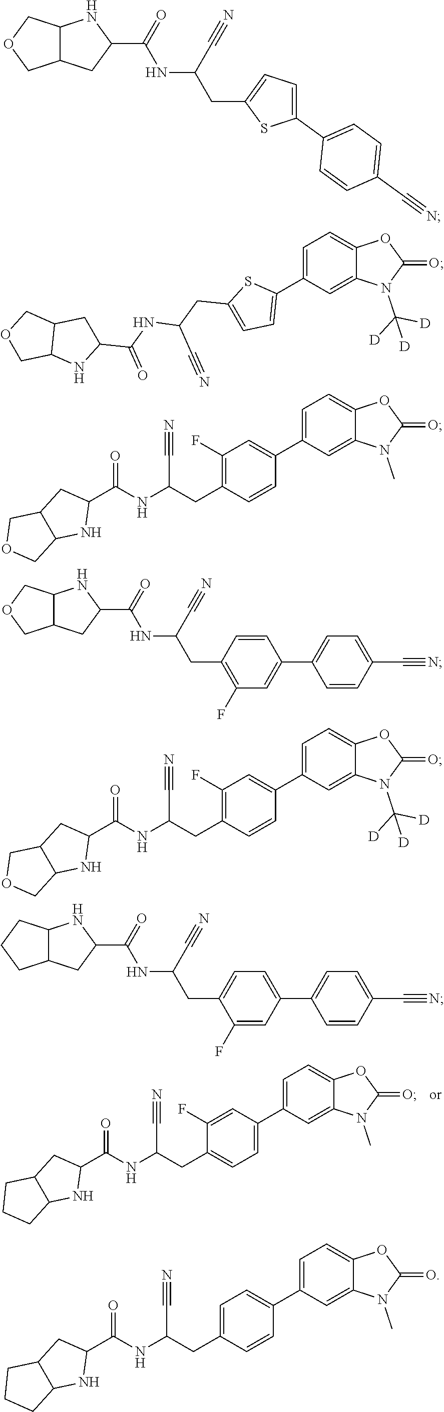

225. The compound of any one of embodiments 172-223, or a pharmaceutically acceptable salt or deuterated form thereof, wherein the compound is:

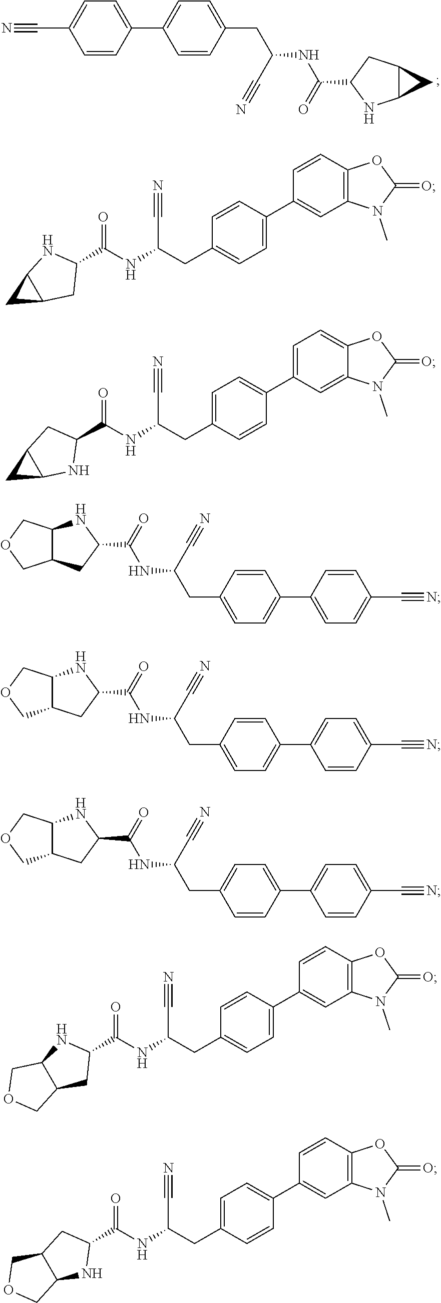

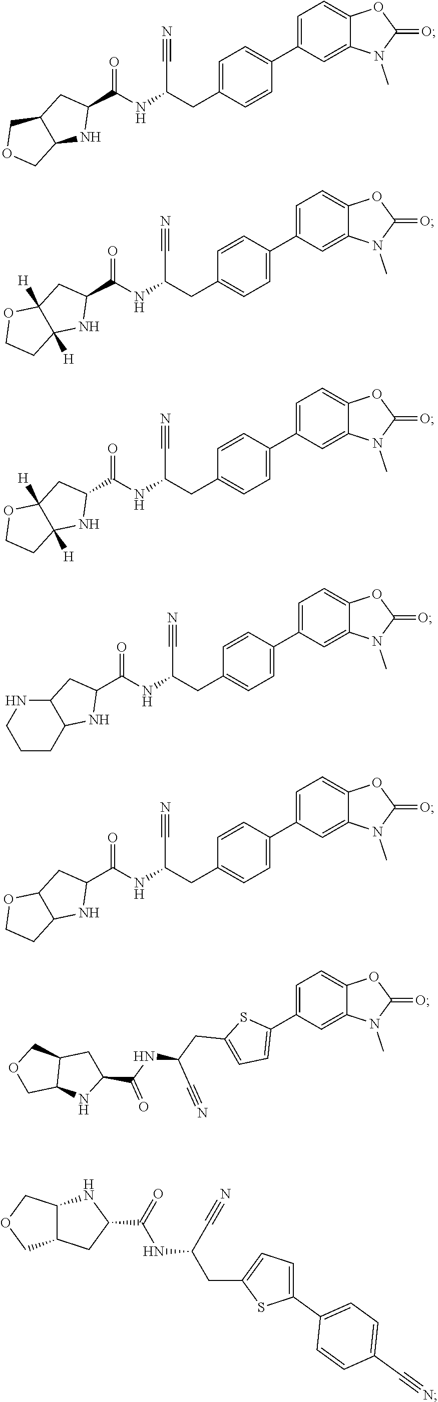

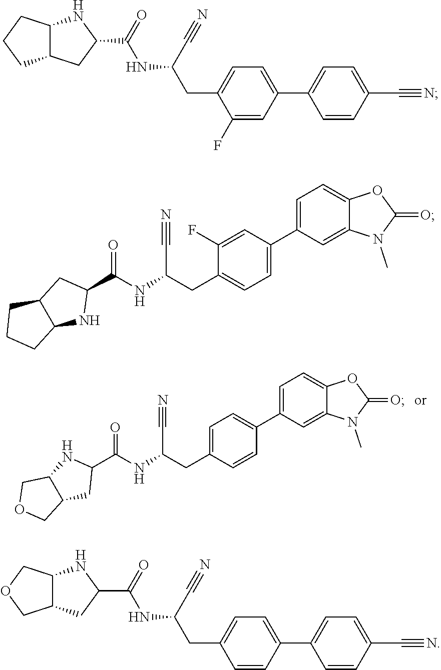

226. A compound having the following chemical formula:

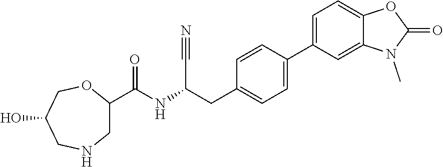 [SMILES: Cn1c(=O)oc2ccc(-c3ccc(C[C@@H](C#N)NC(=O)C4CNC[C@H](O)CO4)cc3)cc21]

or a pharmaceutically accept salt or deuterated form thereof.
227. A compound having the following chemical formula:

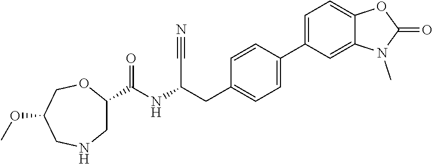 [SMILES: CO[C@H]1CNC[C@@H](C(=O)N[C@H](C#N)Cc2ccc(-c3ccc4oc(=O)n(C)c4c3)cc2)OC1]

or a pharmaceutically accept salt or deuterated form thereof.
228. A method for treating an obstructive disease of the airway in a patient in need thereof, comprising, administering to the patient for an administration period, a composition comprising an effective amount of a compound of any one of embodiments 1-227.
229. The method of embodiment 228, wherein the obstructive disease of the airway is asthma, chronic obstructive pulmonary disease (COPD), bronchitis, emphysema, cystic fibrosis (CF), bronchiectasis, sarcoidosis; alpha-1 antitrypsin (A1AT) deficiency, farmer's lung and related diseases, hypersensitivity pneumonitis, lung fibrosis, complications of lung transplantation, vasculitic and thrombotic disorders of the lung vasculature, pulmonary hypertension, pulmonary hypertension due to left heart disease, pulmonary hypertension associated with chronic lung diseases, antitussive activity including treatment of chronic cough associated with inflammatory and secretory conditions of the airways, iatrogenic cough, acute and chronic rhinitis including rhinitis medicamentosa, and vasomotor rhinitis; perennial and seasonal allergic rhinitis including rhinitis nervosa (hay fever), nasal polyposis; acute viral infection including the common cold, and infection due to a respiratory virus, acute lung injury, or acute respiratory distress syndrome (ARDS).
230. The method of embodiment 229, wherein the obstructive disease of the airway is asthma.
231. The method of embodiment 229, wherein the obstructive disease of the airway is acute respiratory distress syndrome (ARDS).
232. The method of embodiment 229, wherein the obstructive disease of the airway is bronchitis.
233. The method of embodiment 229, wherein the obstructive disease of the airway is lung fibrosis.
234. The method of embodiment 229, wherein the obstructive disease of the airway is emphysema.
235. The method of embodiment 229, wherein the obstructive disease of the airway is cystic fibrosis (CF).
236. The method of embodiment 229, wherein the obstructive disease of the airway is bronchiectasis.
237. The method of embodiment 229, wherein the obstructive disease of the airway is sarcoidosis.
238. The method of embodiment 229, wherein the obstructive disease of the airway is alpha-1 antitrypsin (A1AT) deficiency.
239. The method of embodiment 229, wherein the obstructive disease of the airway is farmer's lung.
240. The method of embodiment 229, wherein the obstructive disease of the airway is hypersensitivity pneumonitis.
241. The method of embodiment 229, wherein the obstructive disease of the airway is a complication of lung transplantation.
242. The method of embodiment 229, wherein the obstructive disease of the airway is a vasculitic or thrombotic disorder of the lung vasculature.
243. The method of embodiment 229, wherein the obstructive disease of the airway is pulmonary hypertension.
244. The method of embodiment 229, wherein the obstructive disease of the airway is iatrogenic cough.
245. The method of embodiment 229, wherein the obstructive disease of the airway is iatrogenic cough.
246. The method of embodiment 229, wherein the obstructive disease of the airway is acute rhinitis.
247. The method of embodiment 229, wherein the obstructive disease of the airway is chronic rhinitis.
248. The method of embodiment 229, wherein the obstructive disease of the airway is nasal polyposis.
249. The method of embodiment 229, wherein the obstructive disease of the airway is pulmonary hypertension due to left heart disease.
250. The method of embodiment 229, wherein the obstructive disease of the airway is pulmonary hypertension associated with chronic lung diseases.
251. The method of embodiment 230, wherein the asthma is bronchial, allergic, intrinsic, extrinsic, exercise-induced or drug-induced asthma.
252. The method of embodiment 232, wherein the bronchitis is infectious bronchitis or eosinophilic bronchitis.
253. The method of embodiment 233, wherein the lung fibrosis is idiopathic pulmonary fibrosis, cryptogenic fibrosing alveolitis, idiopathic interstitial pneumonia, or fibrosis complicating anti-neoplastic therapy or chronic infection.
254. The method of embodiment 236, wherein the bronchiectasis is non-cystic fibrosis bronchiectasis (NCFBE).
255. The method of embodiment 236, wherein the bronchiectasis is associated with cystic fibrosis.
256. The method of embodiment 243, wherein the pulmonary hypertension is pulmonary arterial hypertension.
257. A method for treating cystic fibrosis in a patient in need thereof, comprising, administering to the patient for an administration period, a composition comprising an effective amount of a compound of any one of embodiments 1-227.
258. The method of embodiment 255, wherein the treating comprises improving the lung function of the patient, as compared to the lung function of the patient prior to the administration period.
259. The method of embodiment 258, wherein improving lung function of the patient comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75%)}), as compared to the respective value for the patient prior to the administration period.
260. The method of embodiment 258 or 259, wherein the lung function is measured by spirometry.
261. A method for treating bronchiectasis in a patient in need thereof, comprising, administering to the patient for an administration period, a composition comprising an effective amount of a compound of any one of embodiments 1-227.
262. The method of embodiment 261, wherein the bronchiectasis is non-cystic fibrosis bronchiectasis (NCFBE).
263. The method of embodiment 261, wherein the bronchiectasis is associated with cystic fibrosis.
264. The method of any one of embodiments 260-263, wherein treating comprises improving the lung function of the patient, as compared to the lung function of the patient prior to the administration period.
265. The method of embodiment 262, wherein improving lung function of the patient comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75%)}), as compared to the respective value for the patient prior to the administration period.
266. The method of embodiment 264 or 265, wherein the lung function is measured by spirometry.
267. The method of any one of embodiments 261-266, wherein treating comprises decreasing the rate of pulmonary exacerbation, as compared to the rate of pulmonary exacerbation of the patient prior to the administration period.
268. The method of any one of embodiments 189-267, wherein treating comprises increasing the time to first pulmonary exacerbation, as compared to an untreated patient.
269. The method of embodiment 267 or 268, wherein the pulmonary exacerbation is characterized by three or more of the following symptoms exhibited for at least 48 hours by the patient: (1) increased cough; (2) increased sputum volume or change in sputum consistency;
(3) increased sputum purulence; (4) increased breathlessness and/or decreased exercise tolerance; (5) fatigue and/or malaise; (6) hemoptysis.
270. A method for treating chronic rhinosinusitis (CRS) in a patient in need thereof, comprising, administering to the patient for an administration period, a composition comprising an effective amount of a compound of any one of embodiments 1-225.
271. The method of embodiment 270, wherein the chronic rhinosinusitis is chronic rhinosinusitis without nasal polyps (CRSsNP).
272. The method of embodiment 270, wherein the chronic rhinosinusitis is chronic rhinosinusitis with nasal polyps (CRSwNP).
273. The method of any one of embodiments 270-272, wherein the chronic rhinosinusitis is refractory chronic rhinosinusitis.
274. The method of any one of embodiments 270-273, wherein treating comprises reducing, diminishing the severity of, delaying the onset of, or eliminating one or more symptoms of CRS.
275. The method of embodiment 274, wherein the one or more symptoms of CRS is selected from nasal congestion; nasal obstruction; nasal discharge; post-nasal drip; facial pressure; facial pain; facial fullness; reduced smell; depression; mucosal edema; mucopurulent discharge; obstruction of the middle meatus; mucosal changes within the ostiomeatal complex and sinuses; or rhinorrhea.
276. A method for treating hidradenitis supporativa (HS) in a patient in need thereof, comprising, administering to the patient for an administration period, a composition comprising an effective amount of a compound of any one of embodiments 1-227.
277. The method of embodiment 276, wherein the hidradenitis supporativa (HS) is Hurley stage I.
278. The method of embodiment 276, wherein the hidradenitis supporativa (HS) is Hurley stage II.
279. The method of embodiment 276, wherein the hidradenitis supporativa (HS) is Hurley stage III.
280. A method for treating cancer in a patient in need thereof, comprising, administering to the patient for an administration period, a composition comprising an effective amount of a compound of any one of embodiments 1-227.
281. The method of embodiment 280, wherein the cancer is a metastatic cancer.
282. The method of embodiment 281, wherein the metastatic cancer is breast to lung metastatic cancer.
283. The method of embodiment 281, wherein the metastatic cancer comprises metastasis of breast cancer to the brain, bone, pancreas, lymph nodes or liver.
284. The method of embodiment 281, wherein the metastatic cancer comprises metastasis of bone cancer to the lung.
285. The method of embodiment 281, wherein the metastatic cancer comprises metastasis of colorectal cancer to the peritoneum, the pancreas, the stomach, the lung, the liver, the kidney, or the spleen.
286. The method of embodiment 281, wherein the metastatic cancer comprises metastasis of stomach cancer to the mesentery, the spleen, the pancreas, the lung, the liver, the adrenal gland, or the ovary.
287. The method of embodiment 281, wherein the metastatic cancer comprises metastasis of liver cancer to the intestine, spleen, pancreas, stomach, lung, or the kidney.
288. The method of embodiment 281, wherein the metastatic cancer comprises metastasis of lymphoma to the kidney, ovary, liver, bladder, or the spleen.
289. A method for treating lupus nephritis in a patient in need thereof, comprising, administering to the patient for an administration period, a composition comprising an effective amount of a compound of any one of embodiments 1-227.
290. A method for treating rheumatoid arthritis in a patient in need thereof, comprising, administering to the patient for an administration period, a composition comprising an effective amount of a compound of any one of embodiments 1-227.
291. A method for treating inflammatory bowel disease (IBD) in a patient in need thereof, comprising, administering to the patient for an administration period, a composition comprising an effective amount of a compound of any one of embodiments 1-227.
292. The method of embodiment 291, wherein the inflammatory bowel disease (IBD) is Crohn's disease.
293. The method of embodiment 291, wherein the inflammatory bowel disease (IBD) is ulcerative colitis.
294. A method of treating heart failure in a patient in need thereof, comprising, administering to the patient for an administration period, a composition comprising an effective amount of a compound of any one of embodiments 1-227.
295. The method of embodiment 294, wherein the heart failure is heart failure with preserved ejection fraction.
296. The method of embodiment 294, wherein the heart failure is heart failure with reduced ejection fraction.
297. The method of any one of embodiments 228-296, wherein the composition is administered once a day during the administration period.
298. The method of any one of embodiments 228-296, wherein the composition is administered twice a day during the administration period.
299. The method of any one of embodiments 228-296, wherein the composition is administered every other day during the administration period.
300. The method of any one of embodiments 228-296, wherein the composition is administered once per week during the administration period.
301. The method of any one of embodiments 228-300, wherein the composition is an oral dosage form.
302. The method of embodiment 301, wherein the composition is administered orally.
303. The method of any one of embodiments 228-302, wherein the composition comprises from about 10 mg to about 50 mg of the compound.
304. The method of any one of embodiments 228-303, wherein the administration period is from about 1 year to about 30 years.
305. The method of any one of embodiments 228-303, wherein the administration period is from about 1 year to about 20 years.
306. The method of any one of embodiments 228-303, wherein the administration period is from about 1 year to about 15 years.
307. The method of any one of embodiments 228-303, wherein the administration period is from about 1 year to about 10 years.
308. The method of any one of embodiments 228-303, wherein the administration period is from about 1 year to about 5 years.
309. The method of any one of embodiments 228-303, wherein the administration period is from about 1 year to about 5 years.
310. The method of any one of embodiments 228-303, wherein the administration period is from about 1 year to about 3 years.
311. The method of any one of embodiments 228-303, wherein the administration period is from about 2 years to about 10 years.
312. The method of any one of embodiments 228-303, wherein the administration period is from about 2 years to about 8 years.
313. The method of any one of embodiments 228-303, wherein the administration period is from about 2 years to about 5 years.
314. The method of any one of embodiments 228-303, wherein the administration period is from about 2 years to about 4 years.
315. The method of any one of embodiments 228-303, wherein the administration period is from about 2 years to about 3 years.
316. The method of any one of embodiments 228-303, wherein the administration period is at least about 30 days.
317. The method of any one of embodiments 228-303, wherein the administration period is at least about 60 days.
318. The method of any one of embodiments 228-303, wherein the administration period is at least about 90 days.
319. The method of any one of embodiments 228-303, wherein the administration period is at least about 4 months.
320. The method of any one of embodiments 228-303, wherein the administration period is at least about 6 months.
321. The method of any one of embodiments 228-303, wherein the administration period is at least about 8 months.
322. The method of any one of embodiments 228-303, wherein the administration period is at least about 10 months.
323. The method of any one of embodiments 228-303, wherein the administration period is at least about 1 year.
324. The method of any one of embodiments 228-303, wherein the administration period is at least about 2 years.
325. The method of any one of embodiments 228-303, wherein the administration period is at least about 3 years.
326. The method of any one of embodiments 228-303, wherein the administration period is at least about 4 years.
327. The method of any one of embodiments 228-303, wherein the administration period is at least about 5 years.
328. The method of any one of embodiments 228-303, wherein the administration period is at least about 10 years.

### EXAMPLES

The present invention is further illustrated by reference to the following Examples. However, it should be noted that these Examples, like the embodiments described above, are illustrative and are not to be construed as restricting the scope of the invention in any way.

The following abbreviations are used in the Example section, and the Figures referenced therein.
Boc: tert-butoxycarbonyl
Bu₄NF: tetra-n-butylammonium flouride
Bu₃SNH: Tributyltin hydride
DCM: dichloromethane
DIEA: N,N-Diisopropylethylamine
DIPEA: N,N-Diisopropylethylamine
DMF: dimethylformamide
EdCl: 1-ethyl-3-(3-dimethylaminopropyl)carbodiimide
Et: Ethyl
HCOOH: formic acid
HOBT: hydroxybenzotriazole
K₃PO₄: tripotassium phosphate
LiHMDS: lithium bis(trimethylsilyl)amide
mCPBA: meta-chlorperoxybenzoic acid
Pd(dtbpf)Cl₂: 1,1′-Bis(diphenylphosphino)ferrocene]palladium(II) dichloride
RT: room temperature
TBDPS: tert-butyldiphenylsilyl
TBTU: 2-(1H-Benzotriazole-1-yl)-1,1,3,3,-tetramethylaminium tetraflouroborate
t-BuOK: potassium tert-butoxide
THF: tetrahydrofuran

### Example 1—Synthesis of IWD-002

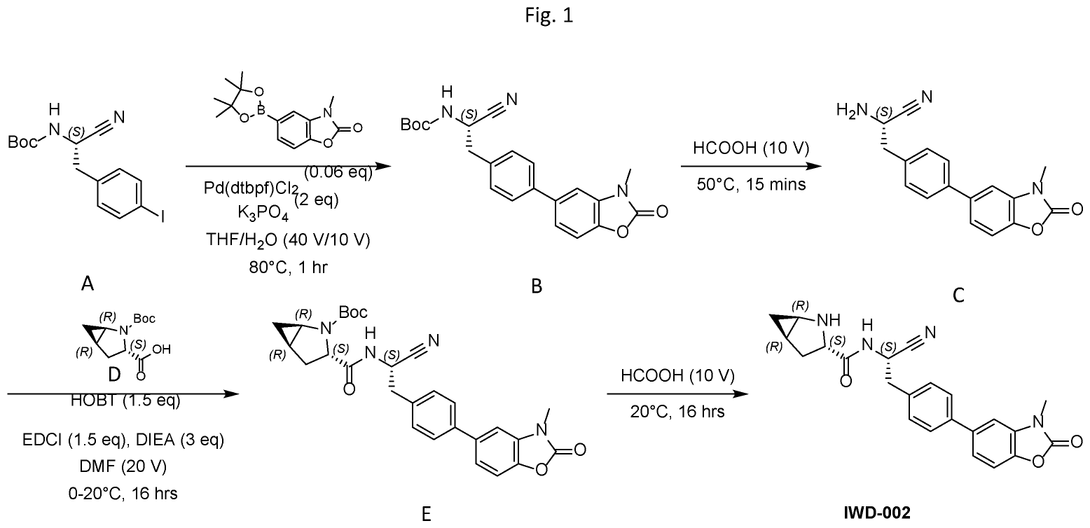

A synthesis route undertaken for IWD-002 (1R,3S,5R)—N—((S)-1-cyano-2-(4-(3-methyl-2-oxo-2,3-dihydrobenzo[d]oxazol-5-yl)phenyl)ethyl)-2-azabicyclo[3.1.0]hexane-3-carboxamide) is provided below, and also shown in FIG. 1.

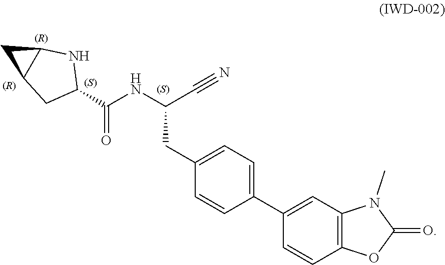

To a solution of Compound A (600 mg, 1.61 mmol) and 3-methyl-6-(4,4,5,5-tetramethyl-1,3,2-dioxaborolan-2-yl)-1,3-benzoxazol-2-one (487.83 mg, 1.77 mmol) in THF (24 mL) was added a solution of K₃PO₄ (684.36 mg, 3.22 mmol) in H₂O (6 mL) at 20° C., then ditert-butyl(cyclopentyl)phosphane; dichloropalladium; iron (63.04 mg, 96.72 umol) was added to the solution under N₂ at 20° C. The reaction mixture was stirred at 80° C. for 1 hrs. LC-MS showed Compound A was consumed completely and product with desired mass was detected. The reaction mixture was quenched by addition water (40 mL) at 20° C., and extracted with ethyl acetate mL (10 mL×3). The combined organic layers were washed with brine (30 mL), dried over Na₂SO₄, filtered and concentrated under reduced pressure to give a residue. The residue was purified by flash silica gel chromatography (ISCO®; 4 g SepaFlash® Silica Flash Column, Eluent of 0˜20% Ethyl acetate/Petroleum ethergradient @ 36 mL/min) to give Compound B (600 mg, 94.60% yield) as brown solid. ¹H NMR: (400 MHz, DMSO-d6) δ ppm 7.86 (br d, J=7.89 Hz, 1H) 7.66 (d, J=8.11 Hz, 2H) 7.58 (s, 1H) 7.33-7.45 (m, 4H) 4.69 (q, J=8.04 Hz, 1H) 3.40 (s, 3H) 3.09 (br d, J=7.67 Hz, 2H) 1.37 (s, 9H)

A solution of Compound B (600 mg, 1.53 mmol) in formic acid (0.5 mL) was stirred at 50° C. for 0.25 hr. LC-MS showed Compound B was consumed completely and product with desired mass was detected. The reaction mixture was concentrated to give residue which was added saturated aqueous solution of NaHCO₃ (30 mL) at 0° C., and extracted with ethyl acetate (10 mL×3). The combined organic layers were washed with brine (20 mL), dried over Na₂SO₄, filtered and concentrated under reduced pressure to give Compound C (400 mg, 89.42% yield) as yellow solid. ¹H NMR: (400 MHz, DMSO-d6) δ ppm 7.65 (d, J=8.11 Hz, 2H) 7.58 (d, J=1.10 Hz, 1H) 7.36-7.46 (m, 4H) 3.95-4.04 (m, 1H) 3.40 (s, 3H) 2.88-3.03 (m, 2H) 2.42 (br s, 2H)

To a solution of Compound D) (116.22 mg, 511.39 umol) in DMF (3 mL) was added 3-(ethyliminomethyleneamino)-N,N-dimethyl-propan-1-amine; hydrochloride (147.05 mg, 767.08 umol), 1-hydroxybenzotriazole (103.65 mg, 767.08 umol) and DIEA (198.28 mg, 1.53 mmol, 267.22 uL) at 20° C. After addition, the mixture was stirred at 20° C. for 30 mins, and then Compound C (150 mg, 511.39 umol) in DMF (3 mL) was added drop-wise at 0° C. The resulting mixture was stirred at 20° C. for 16 hours. LC-MS showed Compound C was consumed completely and product with desired mass was detected. The reaction mixture was quenched by addition water (15 mL) at 20° C., and extracted with ethyl acetate (5 mL×3). The combined organic layers were washed with brine mL (10 mL), dried over Na₂SO₄, filtered and concentrated under reduced pressure to give a residue. The residue was purified by flash silica gel chromatography (ISCO®; 1 g SepaFlash® Silica Flash Column, Eluent of 0˜40% Ethyl acetate/Petroleum ether gradient @ 36 mL/min) to give Compound E (225 mg, 87.55% yield) as white solid. ¹H NMR: (400 MHz, DMSO-d6) δ ppm 8.68 (d, J=8.55 Hz, 1H) 7.66 (d, J=8.11 Hz, 2H) 7.57 (s, 1H) 7.36-7.45 (m, 4H) 4.94-5.12 (m, 1H) 3.68-3.91 (m, 1H) 3.40 (s, 3H) 3.17 (br d, J=8.33 Hz, 2H) 2.11-2.19 (m, 1H) 1.49 (br dd, J=3.40, 1.64 Hz, 3H) 1.32 (br s, 9H) 0.63-0.74 (m, 1H) 0.38 (td, J=4.93, 2.41 Hz, 1H)

A solution of Compound E (100 mg, 198.98 umol) in formic acid (1 mL) was stirred at 20° C. for 16 hours. LC-MS showed Compound E was consumed completely and product with desired mass was detected. The reaction mixture was concentrated under reduced pressure to give a residue. The residue was purified by reversed-phase HPLC (0.1% FA condition) to give IWD-002 (14.5 mg, 16.12% yield) as white solid. Column: Phenomenex Luna C18 75*30 mm*3 um; mobile phase: [water (FA)-ACN]; B %: 10%-50%, 8 mins. ¹H NMR: (400 MHz, DMSO-d6) δ ppm 8.58 (d, J=8.63 Hz, 1H) 8.15 (s, 1H) 7.66 (d, J=8.25 Hz, 2H) 7.58 (d, J=1.13 Hz, 1H) 7.34-7.44 (m, 4H) 5.00 (q, J=7.88 Hz, 1H) 3.40 (s, 3H) 3.31 (t, J=8.63 Hz, 1H) 3.19 (d, J=8.25 Hz, 2H) 2.75 (td, J=5.91, 2.69 Hz, 1H) 2.03 (dd, J=12.26, 8.25 Hz, 1H) 1.50 (ddd, J=12.41, 8.91, 5.32 Hz, 1H) 1.22-1.33 (m, 1H) 0.29-0.41 (m, 2H)

### Example 2-Synthesis of (2S,3αR,6αS)—N—((S)-1-cyano-2-(4′-cyano-[1,1′-biphenyl]-4-yl)ethyl)hexahydro-1H-furo[3,4-b]pyrrole-2-carboxamide (IWD-004)

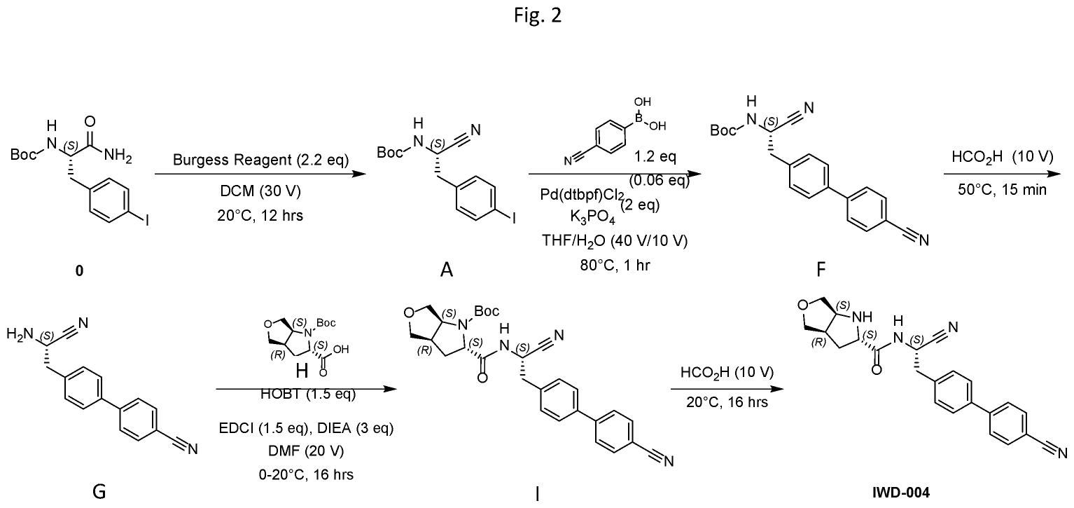

A synthesis route undertaken for IWD-004 ((2S,3αR,6αS)—N—((S)-1-cyano-2-(4′-cyano-[1,1′-biphenyl]-4-yl)ethyl)hexahydro-1H-furo[3,4-b]pyrrole-2-carboxamide) is provided below and is summarized in FIG. 2. IWD-004 is also depicted below.

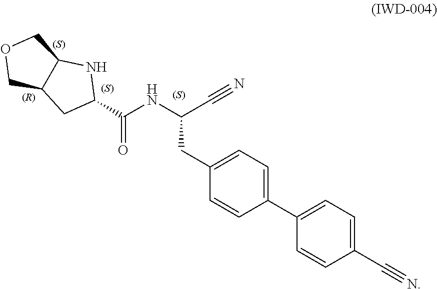

To a solution of Compound 0 (350 mg, 896.94 umol) in CH₂Cl₂ (10.5 mL) was added a solution of methoxycarbonyl-(triethylammonio) sulfonyl-azanide (470.24 mg, 1.97 mmol) in CH₂Cl₂ (1 mL) at 0° C. The mixture was stirred at 20° C. for 16 hr. LCMS showed the reaction was completed. The reaction mixture was quenched by addition water (20 mL) and extracted with ethyl acetate (20 mL×3). The combined organic layers were washed with brine (20 mL), dried over anhydrous Na₂SO₄, filtered and the filtrate was concentrated under reduced pressure to give a residue. The residue was purified by column chromatography on silica gel eluted with ethyl acetate in petroleum ether from 0% to 16% to afford Compound A (430 mg, 82.04% yield) as a white solid. ¹H NMR: (400 MHz, DMSO-d6) δ=7.79 (br d, J=8.03 Hz, 1H) 7.68 (d, J=8.16 Hz, 2H) 7.12 (d, J=8.28 Hz, 2H) 4.64 (q, J=7.86 Hz, 1H) 2.92-3.07 (m, 2H) 1.35 (s, 9H)

To a solution of Compound A (300 mg, 806.02 umol) and (4-cyanophenyl) boronic acid (142.12 mg, 967.22 umol) in THF (12 mL) was added K₃PO₄ (342.18 mg, 1.61 mmol) in water (3 mL) at 20° C., the mixture was degassed and purged with N₂ for 3 times, and then was added ditert-butyl(cyclopentyl)phosphane; dichloropalladium; iron (31.52 mg, 48.4 umol) under N₂ atmosphere. The mixture was stirred at 80° C. for 1 hour. LCMS showed the reaction was completed. The reaction mixture was quenched by addition water (20 mL) at 0° C. and extracted with ethyl acetate (20 mL×3). The combined organic layers were washed with brine (20 mL), dried over anhydrous Na₂SO₄, filtered and the filtrate was concentrated under reduced pressure to give a residue. The residue was purified by column chromatography on silica gel eluted with ethyl acetate in petroleum ether from 0% to 16% to afford Compound F (430 mg, 82.04% yield) as a white solid. ¹H NMR: (400 MHz, DMSO-d6) δ=7.86-7.95 (m, 4H) 7.84 (br d, J=8.13 Hz, 1H) 7.73 (m, J=8.25 Hz, 2H) 7.45 (m, J=8.13 Hz, 2H) 4.71 (q, J=7.71 Hz, 1H) 3.32 (s, 1H) 3.05-3.18 (m, 2H) 1.36 (s, 9H) 0.84 (br d, J=6.88 Hz, 1H).

A solution of Compound F (80 mg, 230.28 μmol) in HCOOH (0.8 mL) was stirred at 50° C. for 0.25 hours. LCMS showed the reaction was completed. The mixture was concentrated under reduced pressure to give the residue. The residue was adjusted to pH=8 with aqueous solution of NaHCO₃ and extracted with ethyl acetate (20 mL×3). The combined organic layers were washed with brine (20 mL), dried over anhydrous Na₂SO₄, filtered and concentrated under reduced pressure to give Compound G (112 mg, 87.41% yield) as a yellow solid. ¹H NMR: (400 MHz, DMSO-d6) δ=7.87-7.94 (m, 4H) 7.71-7.75 (m, 2H) 7.42-7.47 (m, 2H) 3.96-4.05 (m, 1H) 3.34 (s, 1H) 3.08-3.26 (m, 1H) 2.83-3.07 (m, 2H)

To a solution of Compound G (88.43 mg, 343.72 μmol) in DMF (1.8 mL) was added DIEA (133.27 mg, 1.03 mmol, 179.61 uL), EDCI (98.84 mg, 515.58 umol) and HOBt (69.67 mg, 515.58 μmol) at 20° C., then a solution of 4-[4-[(2S)-2-amino-2-cyanoethyl]phenyl]benzonitrile (85 mg, 343.72 μmol) in DMF (0.2 mL) was added at 0° C. The reaction mixture was stirred at 20° C. for 16 hours. LCMS showed the reaction was completed. The reaction mixture was quenched by addition water (5 mL) at 0° C. and extracted with ethyl acetate (5 mL×3). The combined organic layers were washed with brine (5 mL), dried over anhydrous Na₂SO₄, filtered and concentrated under reduced pressure to give a residue. The residue was purified by prep-TLC (petroleum ether:ethyl acetate=1:4) to give Compound I (110 mg, 59.79% yield) as a white solid. ¹H NMR: (400 MHz, DMSO-d6) δ=1.22 (s, 5H) 1.21-1.27 (m, 1H) 1.37 (s, 3H) 1.77-1.95 (m, 2H) 2.72-2.90 (m, 1H) 3.11-3.23 (m, 2H) 3.43-3.50 (m, 2H) 3.52-3.59 (m, 1H) 3.73 (br d, J=9.43 Hz, 1H) 4.14 (dd, J=8.44, 2.52 Hz, 1H) 4.22-4.32 (m, 1H) 4.33-4.40 (m, 1H) 4.99 (d, J=7.67 Hz, 1H) 7.42-7.49 (m, 2H) 7.73 (d, J=8.11 Hz, 2H) 7.84-7.95 (m, 4H)

A solution of Compound I (100 mg, 205.52 umol) in HCOOH (1 mL) was stirred at 20° C. for 16 hours. LCMS showed the reaction was completed. The reaction was concentrated under reduced pressure to give a residue. The residue was purified by prep-HPLC (FA condition) to give IWD-004 (15 mg, 18.19% yield, 96.3% purity) as a white solid. Column: Phenomenex Luna C18 75*30 mm*3 um; mobile phase: [water (FA)-ACN]; B %: 20%-60%, 8 mins. ¹H NMR: (400 MHz, METHANOL-d4) δ=8.36 (br s, 1H) 7.81 (s, 4H) 7.69 (d, J=8.11 Hz, 2H) 7.45 (d, J=8.11 Hz, 2H) 5.11 (t, J=7.67 Hz, 1H) 4.90 (s, 5H) 4.20 (t, J=6.14 Hz, 1H) 3.87-3.98 (m, 2H) 3.67-3.77 (m, 2H) 3.63 (dd, J=10.52, 5.26 Hz, 1H) 3.17-3.30 (m, 2H) 2.93-3.02 (m, 1H) 2.07-2.15 (m, 1H) 1.99-2.07 (m, 1H)

### Example 3-Synthesis of (2S,3αR,6αS)—N—((S)-1-cyano-2-(4-(3-methyl-2-oxo-2,3-dihydrobenzo[d]oxazol-5-yl)phenyl)ethyl)hexahydro-1H-furo[3,4-b]pyrrole-2-carboxamide (IWD-005)

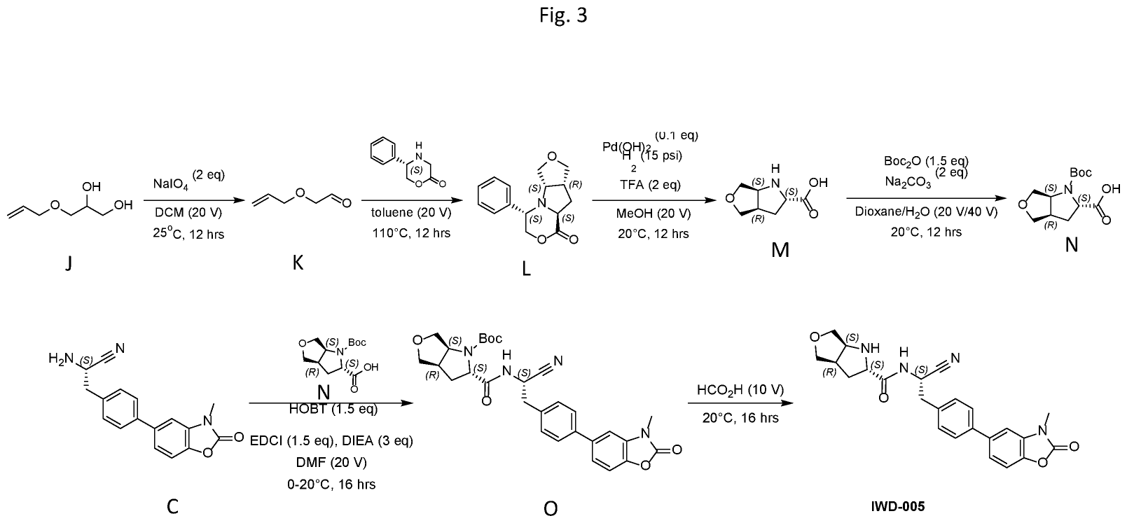

A synthesis route undertaken for (2S,3αR,6αS)—N—((S)-1-cyano-2-(4-(3-methyl-2-oxo-2,3-dihydrobenzo[d]oxazol-5-yl)phenyl)ethyl)hexahydro-1H-furo[3,4-b]pyrrole-2-carboxamide (IWD-005) is discussed below, and is also provided in FIG. 3.

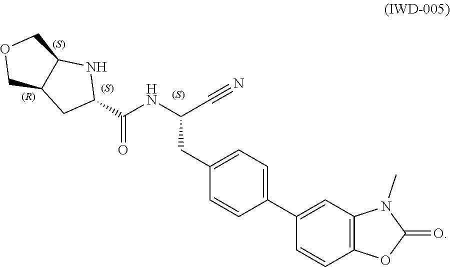

To a mixture of NaIO₄ (0.65 M, 40.00 mL) and SiO₂ (9.09 g, 151.33 mmol) in DCM (80 mL) was added Compound J (4 g, 30.27 mmol) in one portion at 0° C. under N₂. The mixture was stirred at 20° C. for 2 hours. TLC showed the reaction was completed. The reaction mixture was filtered and the filtrate was extracted with CH₂Cl₂ (40 mL×3). The combined organic phase was washed with brine (40 mL×3), dried with anhydrous Na₂SO₄, filtered and concentrated in vacuum to give Compound K (2.5 g, 82.50% yield) as a colorless oil. ¹H NMR: (400 MHz, DMSO-d6) δ ppm 4.03 (dt, J=5.47, 1.39 Hz, 2H), 4.16 (s, 2H), 5.15-5.21 (m, 1H), 5.28 (dd, J=17.26, 1.75 Hz, 1H), 5.82-5.98 (m, 1H), 9.59 (s, 1H).

To a mixture of (5S)-5-phenylmorpholin-2-one (1 g, 5.64 mmol) and Compound K (847.48 mg, 8.47 mmol) in toluene (20 mL) at 25° C. under N₂. The mixture was stirred at 110° C. for 16 hours. LCMS showed the starting material was consumed completely. The reaction mixture was concentrated in vacuum to give product. The crude product was purified by silica gel chromatography (eluted with Petroleum ether/Ethyl acetate=2:1) to give Compound L (1.1 g, 75.17% yield) as a light yellow solid. ¹H NMR: (400 MHz, DMSO-d6) δ ppm 1.85 (d, J=6.13 Hz, 1H), 2.44-2.50 (m, 1H), 2.72-2.84 (m, 1H), 3.13-3.21 (m, 1H), 3.24-3.31 (m, 1H), 3.39-3.52 (m, 2H), 3.59 (dd, J=8.82, 3.19 Hz, 1H), 3.96-4.03 (m, 1 H), 4.25 (s, 1H), 4.27 (d, J=1.38 Hz, 1H), 4.32 (dd, J=7.38, 4.00 Hz, 1H), 7.27-7.34 (m, 1H), 7.38 (t, J=7.38 Hz, 2H), 7.43-7.50 (m, 2H).

To a mixture of Pd (OH) 2 (300 mg, 427.23 umol) and TFA (527.67 mg, 4.63 mmol) in ethyl acetate (10 mL) was added Compound L (600 mg, 2.31 mmol) at 25° C. under H₂ (15 psi). The mixture was stirred at 25° C. for 12 hours. LCMS showed the starting material was consumed completely. The reaction mixture was filtered and the filter was concentrated to give Compound M (350 mg, 96.24% yield) as a white solid. ¹H NMR: (400 MHz, DMSO-d6) δ ppm 2.14-2.23 (m, 2H), 2.98-3.09 (m, 1H), 3.57-3.66 (m, 2H), 3.78 (dd, J=9.26, 2.50 Hz, 1H), 4.04 (br d, J=10.88 Hz, 1H), 4.26 (t, J=8.57 Hz, 1H), 4.33 (br t, J=6.13 Hz, 1H), 9.23-9.95 (m, 1H).

To a solution of Compound M (150 mg, 954.40 umol) in dioxane (2 mL) was added a solution of Na₂CO₃ (202.31 mg, 1.91 mmol) in Water (4 mL) at 20° C. A solution of (Boc) 20 (312.44 mg, 1.43 mmol, 328.89 uL) in dioxane (2 mL) was added to the reaction mixture and the mixture was stirred at 20° C. for 16 hours. LC-MS showed Compound M was consumed and product with desired mass was detected. The reaction mixture was quenched by addition water (10 mL) at 20° C., and then extracted with ethyl acetate mL (5 mL×3) mL. The aqueous layer was adjusted to pH 4 with 1N HCl. Then the mixture was extracted with ethyl acetate (10 mL×6), the combined organic layers were washed with brine (20 mL), dried over Na₂SO₄, filtered and concentrated under reduced pressure to give Compound N (100 mg, 40.72% yield) as a colorless oil. ¹H NMR: (400 MHz, DMSO-d6) δ ppm 12.02 (br s, 1H) 4.20-4.36 (m, 2H) 3.78 (br d, J=9.29 Hz, 1H) 3.47-3.66 (m, 3H) 2.89 (br s, 1H) 1.92-2.20 (m, 2H) 1.39 (br s, 9H).

To a solution of Compound N (96.49 mg, 375.02 umol) in DMF (2 mL) was added 3-(ethyliminomethyleneamino)-N,N-dimethyl-propan-1-amine; hydrochloride (107.84 mg, 562.53 umol), 1-hydroxybenzotriazole (76.01 mg, 562.53 umol) and DIEA (145.40 mg, 1.13 mmol, 195.96 uL) at 20° C. After addition, the mixture was stirred at this temperature for 30 mins, and then Compound I (110 mg, 375.02 umol) in DMF (2 mL) was added dropwise at 0° C. The resulting mixture was stirred at 20° C. for 16 hours. LC-MS showed Compound I was consumed completely and product with desired mass was detected. The reaction mixture was quenched by addition water (15 mL) at 20° C. and extracted with ethyl acetate (5 mL×3). The combined organic layers were washed with brine (10 mL), dried over Na₂SO₄, filtered and concentrated under reduced pressure to give a residue. The residue was purified by flash silica gel chromatography (ISCO®; 1 g SepaFlash® Silica Flash Column, Eluent of 0˜60% Ethyl acetate/Petroleum ethergradient @ 36 mL/min) to give Compound ((162 mg, 81.11% yield) as white solid. ¹H NMR: (400 MHz, CHLOROFORM-d) δ ppm 7.51-7.61 (m, 2H) 7.34-7.45 (m, 3H) 7.27-7.33 (m, 2H) 7.13 (d, J=1.32 Hz, 1H) 5.02-5.20 (m, 1H) 4.45 (d, J=7.89 Hz, 1H) 4.16-4.23 (m, 1H) 3.90 (dd, J=9.98, 2.74 Hz, 1H) 3.76 (d, J=9.21 Hz, 1H) 3.69 (dt, J=9.92, 6.55 Hz, 1H) 3.57 (dd, J=9.32, 4.93 Hz, 1H) 3.46 (s, 3H) 3.02-3.20 (m, 3H) 2.46 (dd, J=12.72, 8.33 Hz, 1H) 1.79-1.89 (m, 1H) 1.37-1.53 (m, 9H).

A solution of Compound O (150 mg, 281.64 umol) in formic acid (1.5 mL) was stirred at 20° C. for 16 hours. LC-MS showed Compound (was consumed completely and product with desired mass was detected. The reaction mixture was concentrated under reduced pressure to give a residue. The residue was purified by reversed-phase HPLC (0.1% FA condition) to give IWD-005 (33.3 mg, 24.29% yield) as white solid. Column: Phenomenex Luna C18 75*30 mm*3 um; mobile phase: [water (FA)-ACN]; B %: 20%-60%, 8 mins. ¹H NMR: (400 MHz, CHLOROFORM-d) δ ppm 8.07 (br s, 1H) 7.82 (br d, J=8.77 Hz, 1H) 7.56 (d, J=8.11 Hz, 2H) 7.37 (d, J=8.11 Hz, 2H) 7.29-7.34 (m, 1H) 7.27-7.29 (m, 1H) 7.14 (d, J=1.53 Hz, 1H) 5.09-5.20 (m, 1H) 3.99 (t, J=7.34 Hz, 1H) 3.76-3.84 (m, 2H) 3.66-3.75 (m, 2H) 3.63 (dd, J=10.19, 4.71 Hz, 1H) 3.47 (s, 3H) 3.16 (d, J=7.02 Hz, 2H) 2.77 (qt, J=7.42, 3.56 Hz, 1H) 2.13 (dt, J=12.93, 7.67 Hz, 1H) 1.97-2.06 (m, 1H).

### Example 4—Synthesis of (S)-2-amino-3-(4-(3-methyl-2-oxo-2,3-dihydrobenzo[d]oxazol-5-yl)phenyl)propanenitrile hydrochloride salt

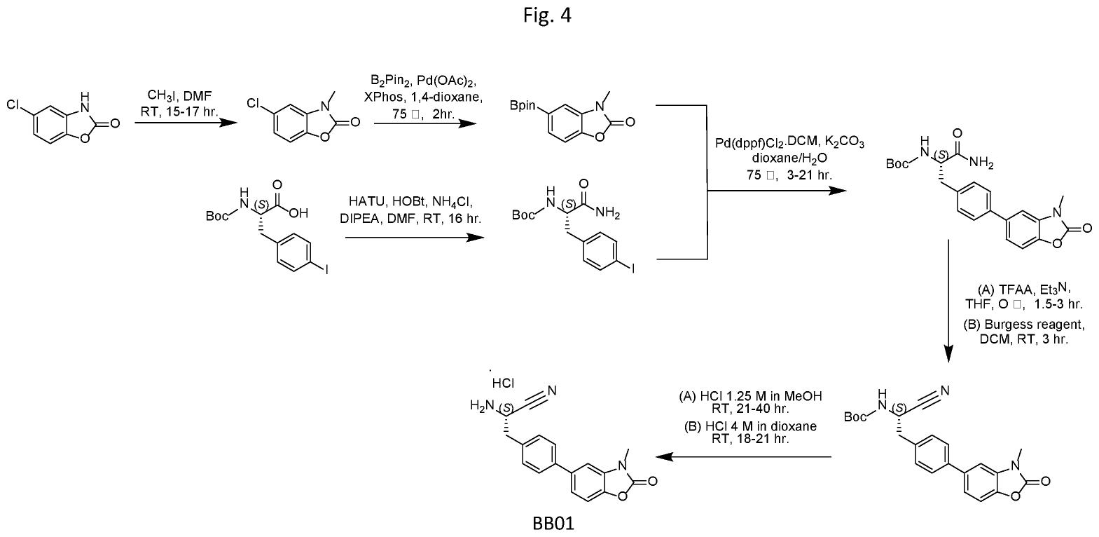

A synthesis scheme for(S)-2-amino-3-(4-(3-methyl-2-oxo-2,3-dihydrobenzo[d]oxazol-5-yl)phenyl)propanenitrile hydrochloride salt (also referred to herein as BB01), which can be used as a reactant for obtaining a compound of Formula (IA) and/or (II), is provided in FIG. 4.

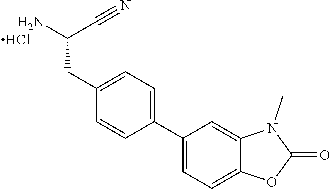 [SMILES: Cl ; Cn1c(=O)oc2ccc(-c3ccc(C[C@H](N)C#N)cc3)cc21]

### (S)-2-amino-3-(4-(3-methyl-2-oxo-2,3-dihydrobenzo[d]oxazol-5-yl)phenyl) propanenitrile hydrochloride (BB-01)

### Example 5—Synthesis of Exemplary Cyclic Bridged α-amino Derivatives

A synthesis scheme for an exemplary cyclic bridged α-amino moiety,

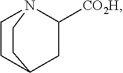 [SMILES: O=C(O)C1CC2CCN1CC2]

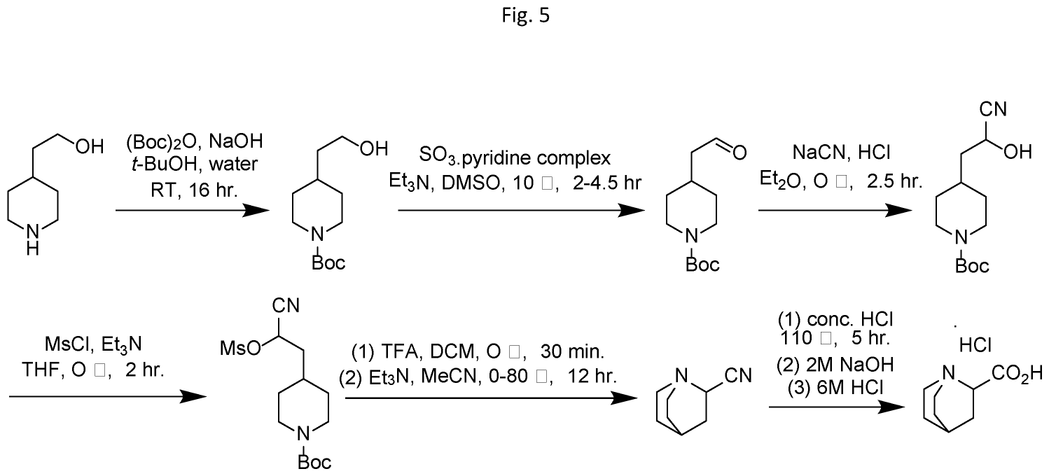

which can be used as a reactant for obtaining a compound of Formula (II), is provided in FIG. 5.

### Example 6—Synthesis of Exemplary Cyclic Sulfonyl α-amino Moieties

Synthesis schemes for an exemplary cyclic sulfonyl α-amino moiety,

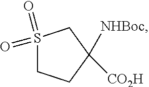 [SMILES: CC(C)(C)OC(=O)NC1(C(=O)O)CCS(=O)(=O)C1]

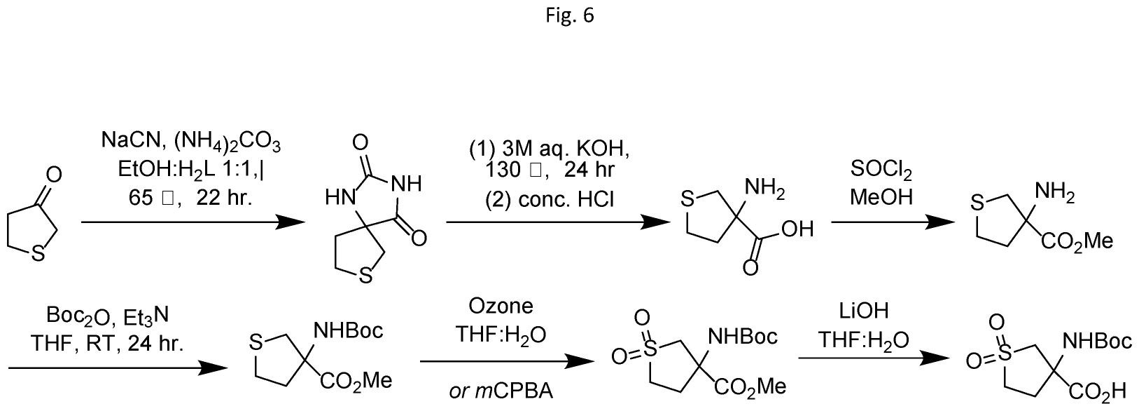

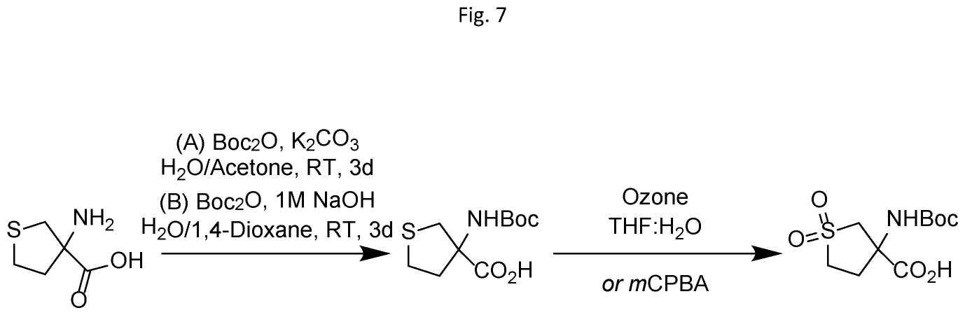

which can be used as a reactant for obtaining a compound of Formula (II), are provided in FIGS. 6 and 7.

### Example 7—Synthesis of an Exemplary Exo-Beta NH₂ Cyclic Derivative Moiety

A synthesis scheme for an exemplary exo-beta NH₂ cyclic moiety,

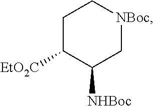 [SMILES: CCOC(=O)[C@@H]1CCN(C(=O)OC(C)(C)C)C[C@H]1NC(=O)OC(C)(C)C]

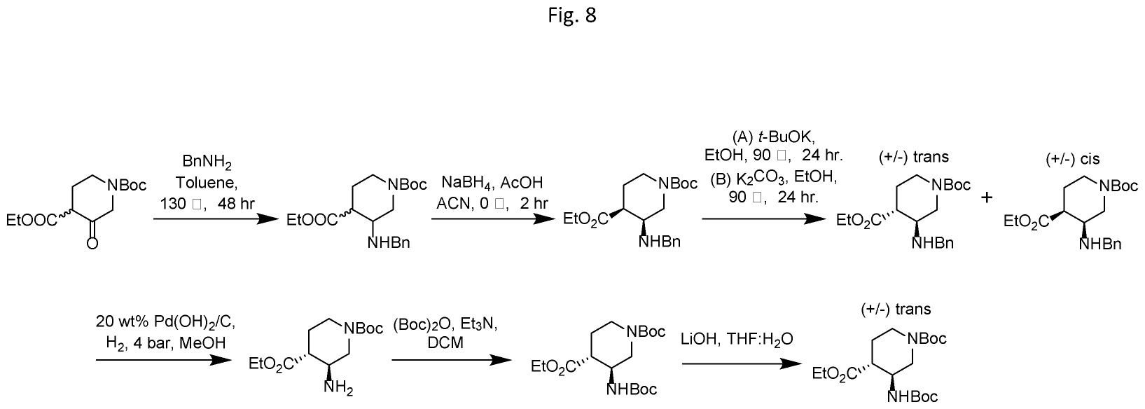

which can be used as a reactant for obtaining a compound of the invention, is provided in FIG. 8.

### Example 8—Synthesis of an Exemplary Beta-Amino Spirocyclic Moiety

A synthesis scheme for an exemplary beta-amino spirocyclic moiety,

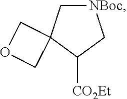 [SMILES: CCOC(=O)C1CN(C(=O)OC(C)(C)C)CC12COC2]

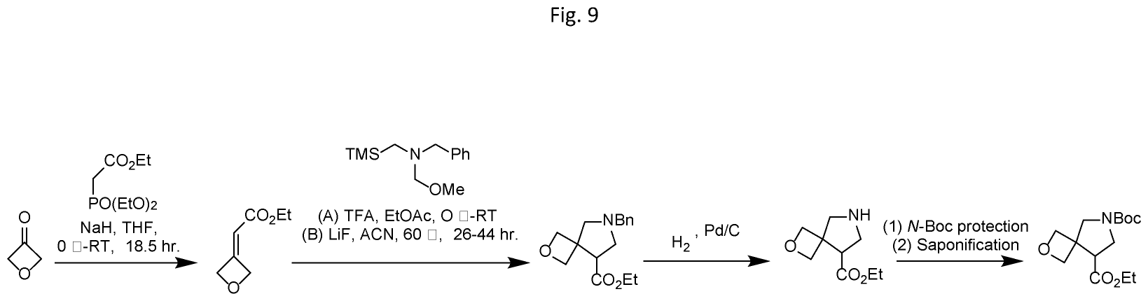

which can be used as a reactant for obtaining a compound of the invention, is provided in FIG. 9.

### Example 9—Synthesis of B1-26 ((S)-3-amino-N-(1-cyano-2-(4-(3-methyl-2-oxo-2,3-dihydrobenzo[d]oxazol-5-yl)phenyl)ethyl)-3-methylbutanamide)

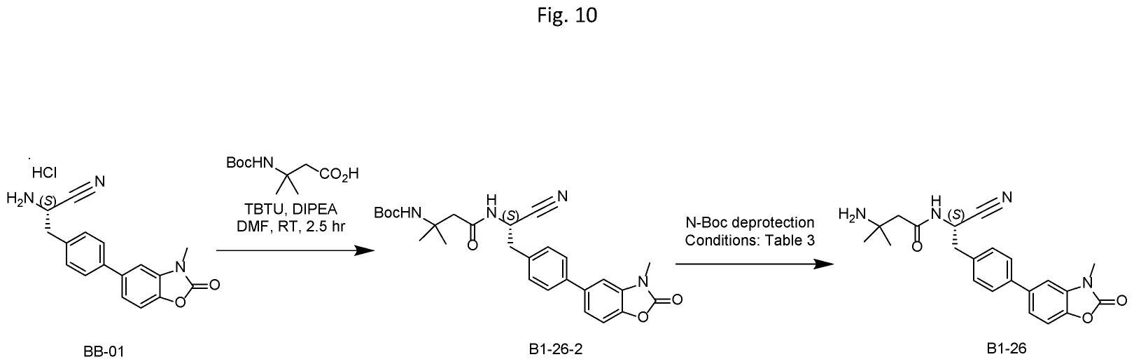

A synthesis scheme for B1-26 ((S)-3-amino-N-(1-cyano-2-(4-(3-methyl-2-oxo-2,3-dihydrobenzo[d]oxazol-5-yl)phenyl)ethyl)-3-methylbutanamide), a DPP1 inhibitor having an exemplary beta-carbon substituted moiety, is provided in FIG. 10.

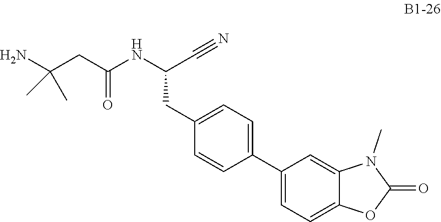 [SMILES: Cn1c(=O)oc2ccc(-c3ccc(C[C@@H](C#N)NC(=O)CC(C)(C)N)cc3)cc21]

The conditions for deprotection of B1-26-2, referenced in FIG. 10 are provided below, in Table 12.

TABLE 12
Conditions for N-Boc deprotection of B1-26-2

| Target | Scale | | Conditions | Purification |
|---|---|---|---|---|
| B1-26 | 12 | mg | B1-26-2 (1 eq.), 1.25M HCl in | Concentration & |
| | | | MeOH (35 eq.), RT, 18 h | trituration |
| B1-26 | 20 | mg | B1-26-2 (1 eq.), 1.25M HCl in | — |
| | | | MeOH (5 eq.), MeOH, RT, 18 h | |
| B1-26 | 20 | mg | B1-26-2 (1 eq.), 4M HCl in 1,4- | — |
| | | | dioxane (15 eq.), DCM, RT, | |
| | | | 18 h | |
| B1-26 | 20 | mg | B1-26-2 (1 eq.), formic acid | — |
| | | | (200 eq.), 50° C., 15 min | |
| B1-26 | 10 | mg | B1-26-2 (1 eq.), formic acid | — |
| | | | (200 eq.), 0° C., 15 min | |
| B1-26 | 250 | mg | B1-26-2 (1 eq.), formic acid | (1) Work-up (aq. |

| | |
|---|---|
| (200 eq.), 50° C., 15 min | sat. NaHCO₃) |
| | (2) Purification |
| | over SiO₂ |

### Example 10—Synthesis of B1-18 (N—((S)-1-cyano-2-(4-(3-methyl-2-oxo-2,3-dihydrobenzo[d]oxazol-5-yl)phenyl)ethyl)-2-(morpholin-3-yl)acetamide)

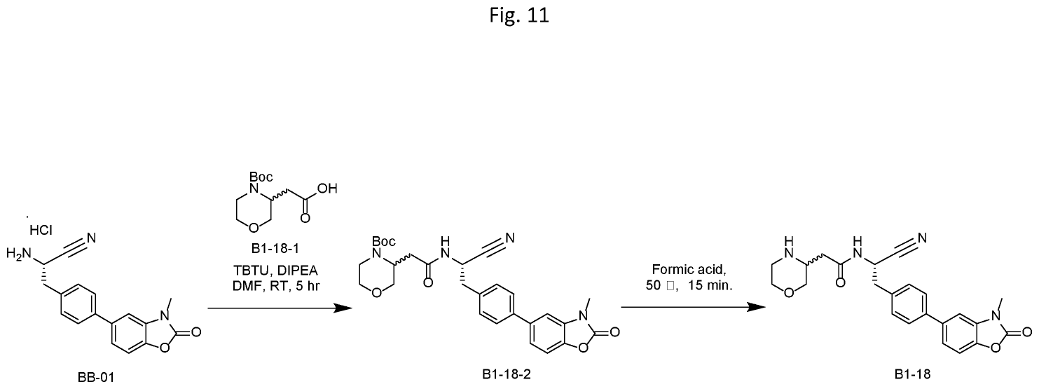

A synthesis scheme for B1-26 ((S)-3-amino-N-(1-cyano-2-(4-(3-methyl-2-oxo-2,3-dihydrobenzo[d]oxazol-5-yl)phenyl)ethyl)-3-methylbutanamide) (shown below), is provided in FIG. 11.

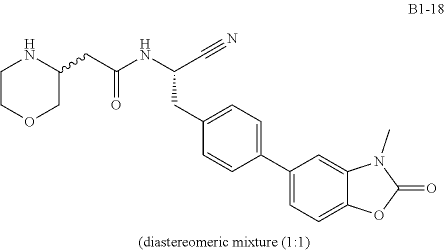 [SMILES: Cn1c(=O)oc2ccc(-c3ccc(C[C@@H](C#N)NC(=O)CC4COCCN4)cc3)cc21]

### Example 11—IC50 Assays

### Mouse DPP1 Enzyme IC50 Assay

Test articles were applied to active mouse DPP1 enzyme (R&D Systems; Minneapolis, MN) in Assay Buffer (50 mM MES pH 5.5, 50 mM NaCl, 5 mM DTT) in a total reaction volume of 125 μL. 25 μL of compound in Assay Buffer plus 5% DMSO was first added to 50 μL of active mouse DPP1 enzyme at a concentration of 62.5 pg/μL and allowed to pre-incubate for 10 minutes at 37° C. after which 50 μL of 1000 μM H-Gly-Arg-AMC substrate (Bachem; St. Torrance, CA) was added, giving final substrate concentration of 400 μM and a final DMSO concentration of 1%. Substrate cleavage was measured for 90 minutes at 37° C., with fluorescence at Excitation/Emission 350/450 nm measured every 5 minutes. DPP1 concentration was interpolated based on its activity relative to a standard curve of recombinant active mouse DPP1 enzyme. IC50 values for each compound were calculated via the XLFit (IDBS Version 5.3.1.3) Add-On to Microsoft Excel using the four parameter fit equation y=(A+((B−A)/(1+((C/x)\hat{ }D)))), which appears as equation number 205 (4 Parameter Logistic Model or Sigmoidal Dose-Response Model) in XLFit. Default constraints were used for each Parameter. IC50 was defined as the compound concentration at which 50% of enzyme activity was inhibited when compared to the no-compound control.

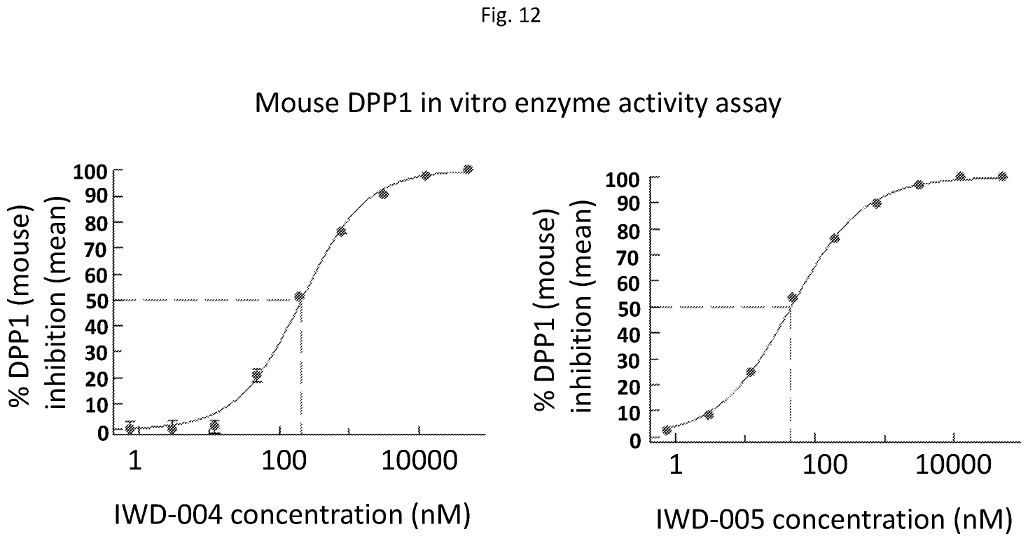

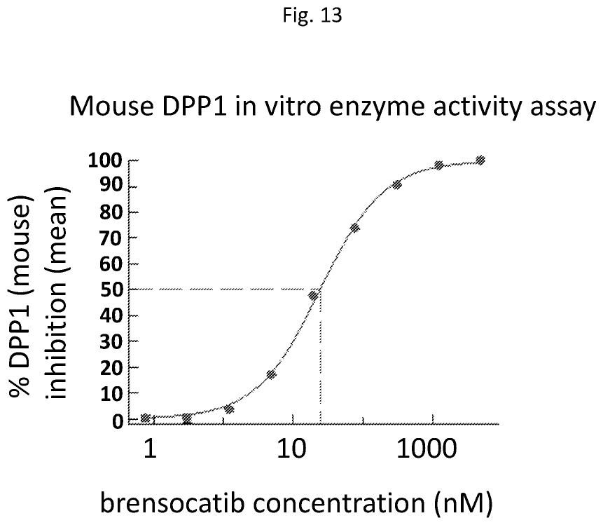

Results are provided in FIG. 12 and FIG. 13, and in Table 13, below.

TABLE 13

| Compound | IC50 (nM) |
|---|---|
| IWD-004 | 209 |
| IWD-005 | 45.6 |
| brensocatib | 24.3 |

### Human DPP1 Enzyme IC50 Assay

Recombinant human DPP1 enzyme (R&D Systems; Minneapolis, MN) was first proteolytically processed into its mature form using recombinant human cathepsin L (R&D Systems) in a buffer consisting of 20 mM citric acid pH 4.5, 150 mM NaCl, 1 mM EDTA and 10 mM DTT. Test articles were applied to activated human DPP1 enzyme in Assay Buffer (25 mM MES pH 6.0, 50 mM NaCl, 5 mM DTT) in a total reaction volume of 125 μL. 25 μL of compound in Assay Buffer plus 5% DMSO was first added to 50 μL of activated human DPP1 enzyme at a concentration of 1 ng/μL and allowed to pre-incubate for 10 minutes at 37° C. after which 50 μL of 1000 μM H-Gly-Arg-AMC substrate (Bachem; St. Torrance, CA) was added, giving final substrate concentration of 400 μM and a final DMSO concentration of 1%. Substrate cleavage was measured for 90 minutes at 37° C., with fluorescence at Excitation/Emission 350/450 nm measured every 5 minutes. DPP1 concentration was interpolated based on its activity relative to a standard curve of activated human recombinant DPP1 enzyme. IC50 values for each compound were calculated via the XLFit (IDBS Version 5.3.1.3) Add-On to Microsoft Excel using the four parameter fit equation y={A+[(B−A)]/[1+((C/x)\hat{ }D)]}, which appears as equation number 205 (4 Parameter Logistic Model or Sigmoidal Dose-Response Model) in XLFit. Default constraints were used for each Parameter. IC50 was defined as the compound concentration at which 50% of enzyme activity was inhibited when compared to the no-compound control.

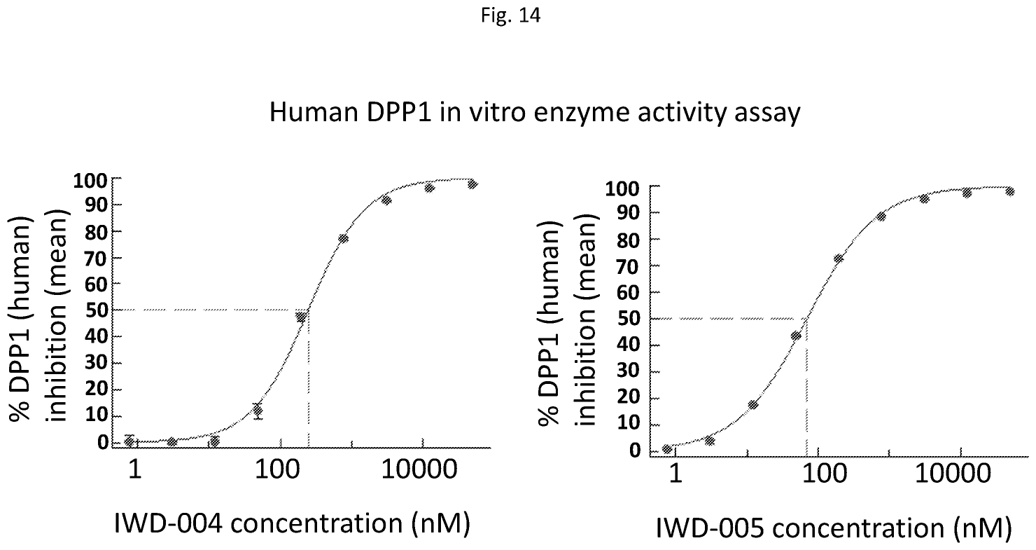

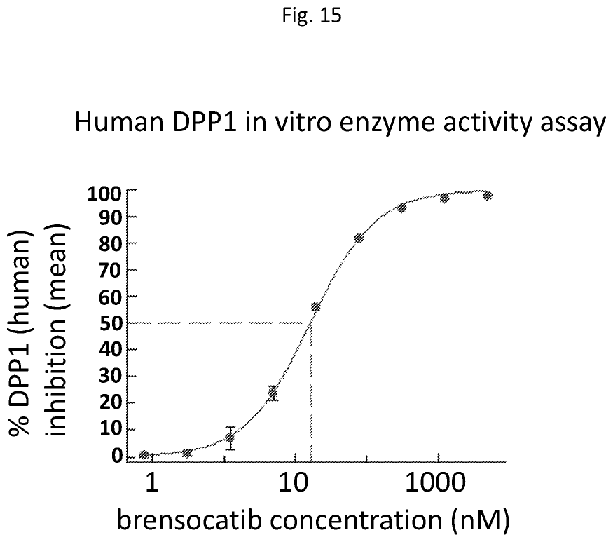

Results are provided in FIG. 14 and FIG. 15 and in Table 14 below.

TABLE 14

| Compound | IC50 (nM) |
|---|---|
| IWD-004 | 245 |
| IWD-005 | 69.2 |
| brensocatib | 16.0 |

### DPP1 Cell IC50 Assay

HL-60 cells (ATCC; Manassas, VA) were maintained in RPMI-1640 supplemented with 20% heat-inactivated FBS and 1× Antibiotic Antimycotic (Cytiva; Marlborough, MA). Media was changed every three to four days and cells were not allowed to exceed 1×10⁶ cells per mL. Prior to assay, cells were collected by centrifugation at 500 rcf for 3 minutes, resuspended in PBS and counted. Cells were diluted in PBS to a concentration of 5×10⁵ live cells per mL and transferred to black 96-well plates for assay, 60 μL per well. Test articles were diluted in PBS plus 0.5% DMSO, and 20 μL was added to each assay well. Compound was allowed to pre-incubate with cells with gentle shaking at 100 rpm for 60 minutes at 37° C. in a cell culture incubator maintained at 5% CO₂, after which 20 μL of 500 μM H-Gly-Phe-AFC substrate (MP Biomedicals; Solon, OH) was added to each well. Plates were returned to the incubator with shaking at 100 rpm for 30 minutes, after which fluorescence was measured at Excitation/Emission 400/505 nm. % Inhibition was calculated from RFU values compared to control cell wells that received only PBS plus 0.5% DMSO. IC50 values for each compound were calculated via the XLFit (IDBS Version 5.3.1.3) Add-On to Microsoft Excel using the four parameter fit equation y=(A+((B−A)/(1+((C/x)\hat{ }D)))), which appears as equation number 205 (4 Parameter Logistic Model or Sigmoidal Dose-Response Model) in XLFit. IC50 was defined as the compound concentration at which 50% of enzyme activity was inhibited when compared to the no-compound control.

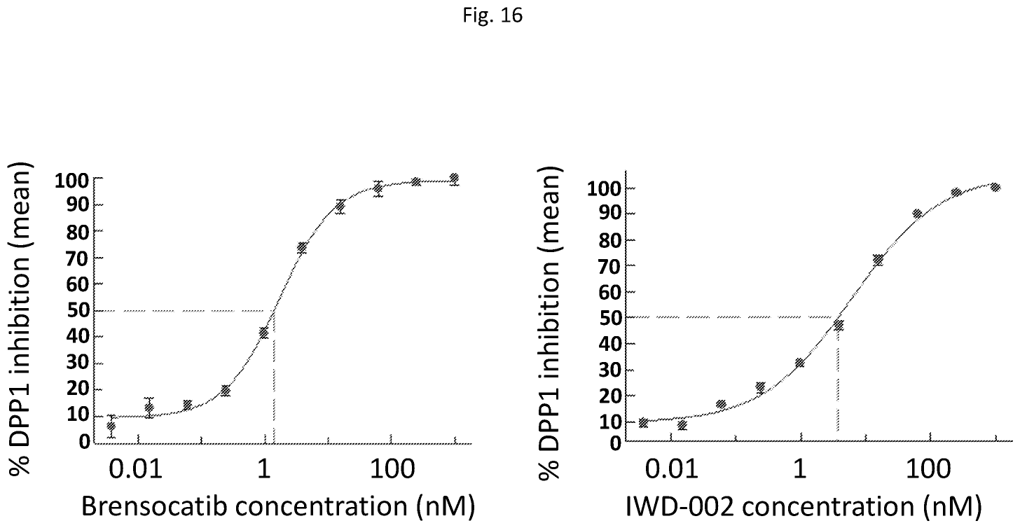

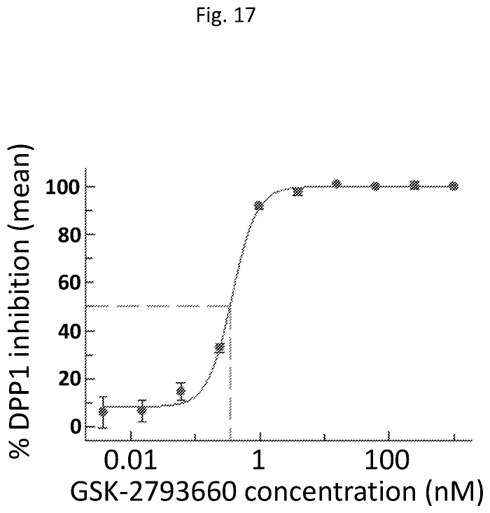

The results of these assays are provided in FIG. 16 and FIG. 17, and Table 15, below.

TABLE 15

| Compound | Cell IC50 (nM) |
|---|---|
| IWD-002 | 3.7 |
| brensocatib | 1.4 |
| GSK-2793660 | 0.34 |

### Example 12—IC 50 Assays

The mouse and human assays described above in Example 11 were performed for the compounds enumerated in Table 16.

TABLE 16

| IC50, | IC50, | Cell | |
|---|---|---|---|
| nM; | nM; | Assay, | |
| Human | Mouse | nM | Compound |
| 19.7 | 27 | 1.5 | 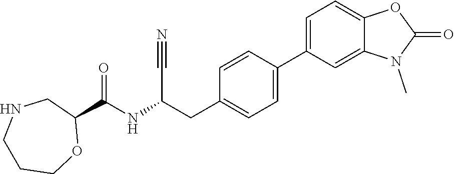 [SMILES: Cn1c(=O)oc2ccc(-c3ccc(C[C@@H](C#N)NC(=O)[C@@H]4CNCCCO4)cc3)cc21] |
| 139.7 | 92.4 | — | 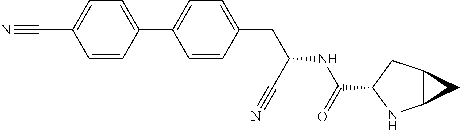 [SMILES: N#Cc1ccc(-c2ccc(C[C@@H](C#N)NC(=O)[C@@H]3C[C@H]4C[C@H]4N3)cc2)cc1] |
| 41.1 | 24 | 3.7 | 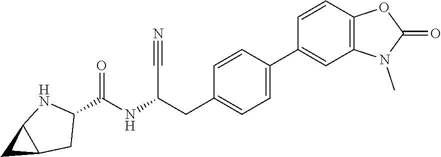 [SMILES: Cn1c(=O)oc2ccc(-c3ccc(C[C@@H](C#N)NC(=O)[C@@H]4C[C@H]5C[C@H]5N4)cc3)cc21] |
| 30.4 | 19.6 | — | 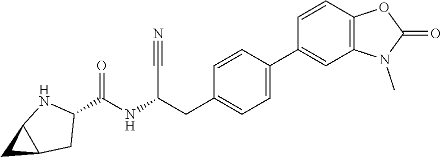 [SMILES: Cn1c(=O)oc2ccc(-c3ccc(C[C@@H](C#N)NC(=O)[C@@H]4C[C@H]5C[C@H]5N4)cc3)cc21] |
| 269.2 | 209 | — | 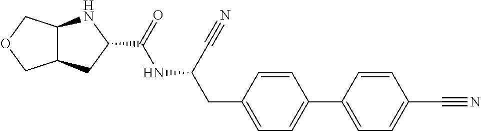 [SMILES: N#Cc1ccc(-c2ccc(C[C@@H](C#N)NC(=O)[C@@H]3C[C@H]4COC[C@H]4N3)cc2)cc1] |
| 69.2 | 45.6 | — | 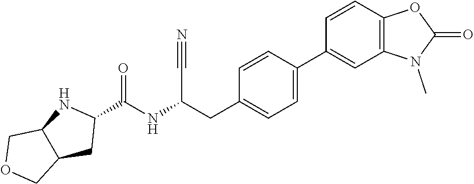 [SMILES: Cn1c(=O)oc2ccc(-c3ccc(C[C@@H](C#N)NC(=O)[C@@H]4C[C@H]5COC[C@H]5N4)cc3)cc21] |
| 188.6 | 131.2 | 26 | 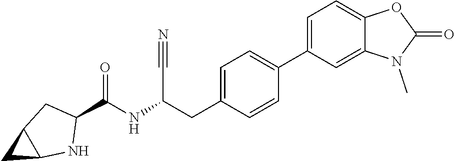 [SMILES: Cn1c(=O)oc2ccc(-c3ccc(C[C@@H](C#N)NC(=O)[C@@H]4C[C@@H]5C[C@@H]5N4)cc3)cc21] |
| 940.1 | — | — | 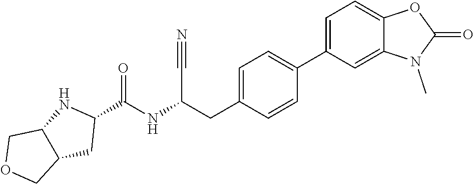 [SMILES: Cn1c(=O)oc2ccc(-c3ccc(C[C@@H](C#N)NC(=O)[C@@H]4C[C@@H]5COC[C@@H]5N4)cc3)cc21] |
| 2608.5 | — | — | 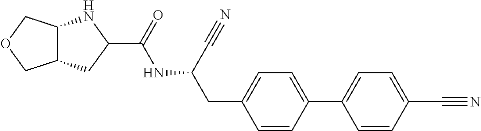 |
| >5000 | >5000 | — | 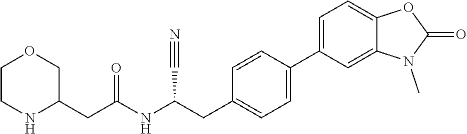 [SMILES: Cn1c(=O)oc2ccc(-c3ccc(C[C@@H](C#N)NC(=O)CC4COCCN4)cc3)cc21] |
| >5000 | >5000 | — | 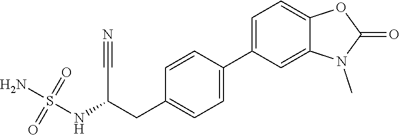 [SMILES: Cn1c(=O)oc2ccc(-c3ccc(C[C@@H](C#N)NS(N)(=O)=O)cc3)cc21] |
| >5000 | >5000 | — | 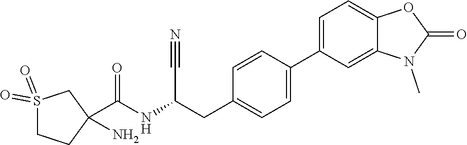 [SMILES: Cn1c(=O)oc2ccc(-c3ccc(C[C@@H](C#N)NC(=O)C4(N)CCS(=O)(=O)C4)cc3)cc21] |
| >5000 | >5000 | — | 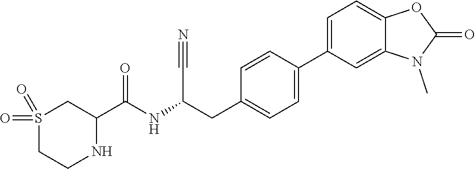 [SMILES: Cn1c(=O)oc2ccc(-c3ccc(C[C@@H](C#N)NC(=O)C4CS(=O)(=O)CCN4)cc3)cc21] |
| >5000 | >5000 | — | 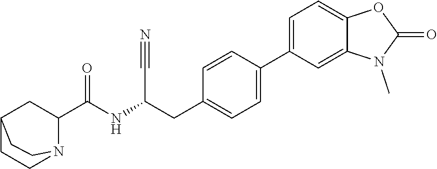 [SMILES: Cn1c(=O)oc2ccc(-c3ccc(C[C@@H](C#N)NC(=O)C4CC5CCN4CC5)cc3)cc21] |
| >5000 | >5000 | — | 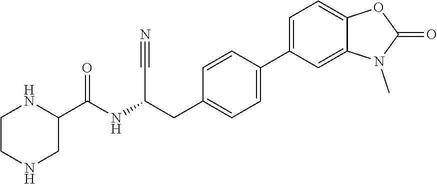 [SMILES: Cn1c(=O)oc2ccc(-c3ccc(C[C@@H](C#N)NC(=O)C4CNCCN4)cc3)cc21] |
| >5000 | >5000 | — | 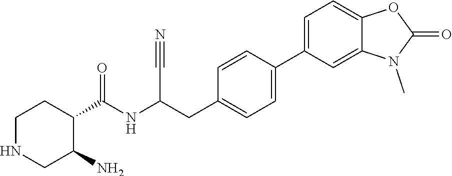 [SMILES: Cn1c(=O)oc2ccc(-c3ccc(CC(C#N)NC(=O)[C@H]4CCNC[C@@H]4N)cc3)cc21] |
| 1308 | 2433 | — | 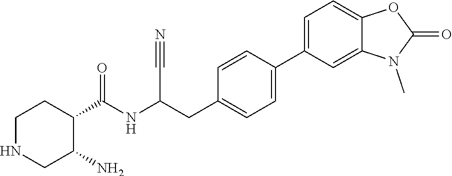 [SMILES: Cn1c(=O)oc2ccc(-c3ccc(CC(C#N)NC(=O)[C@H]4CCNC[C@H]4N)cc3)cc21] |
| 2545 | 3294 | — | 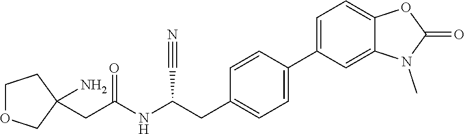 [SMILES: Cn1c(=O)oc2ccc(-c3ccc(C[C@@H](C#N)NC(=O)CC4(N)CCOC4)cc3)cc21] |
| 537 | 306 | 330 | 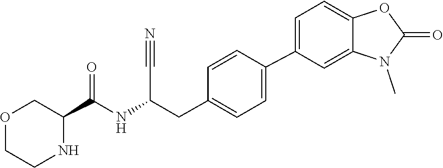 [SMILES: Cn1c(=O)oc2ccc(-c3ccc(C[C@@H](C#N)NC(=O)[C@@H]4COCCN4)cc3)cc21] |
| 2176 | 3164 | — | 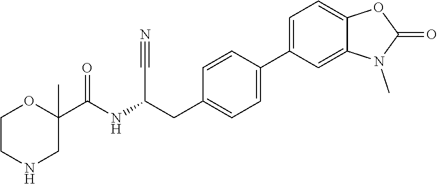 [SMILES: Cn1c(=O)oc2ccc(-c3ccc(C[C@@H](C#N)NC(=O)C4(C)CNCCO4)cc3)cc21] |
| >5000 | >5000 | — | 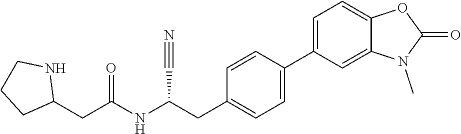 [SMILES: Cn1c(=O)oc2ccc(-c3ccc(C[C@@H](C#N)NC(=O)CC4CCCN4)cc3)cc21] |
| >5000 | >5000 | — | 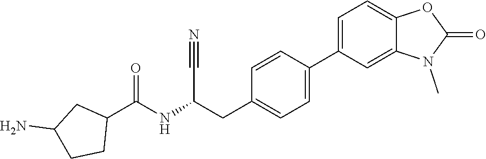 [SMILES: Cn1c(=O)oc2ccc(-c3ccc(C[C@@H](C#N)NC(=O)C4CCC(N)C4)cc3)cc21] |
| >5000 | >5000 | — | 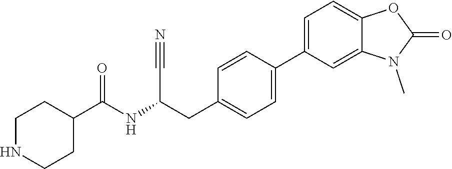 [SMILES: Cn1c(=O)oc2ccc(-c3ccc(C[C@@H](C#N)NC(=O)C4CCNCC4)cc3)cc21] |
| 3656 | 2768 | — | 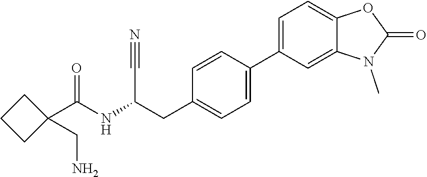 [SMILES: Cn1c(=O)oc2ccc(-c3ccc(C[C@@H](C#N)NC(=O)C4(CN)CCC4)cc3)cc21] |
| >5000 | >5000 | — | 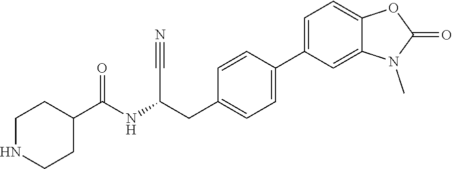 [SMILES: Cn1c(=O)oc2ccc(-c3ccc(C[C@@H](C#N)NC(=O)C4CCNCC4)cc3)cc21] |
| 1269 | 825 | — | 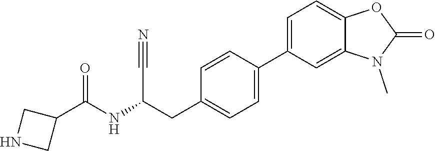 [SMILES: Cn1c(=O)oc2ccc(-c3ccc(C[C@@H](C#N)NC(=O)C4CNC4)cc3)cc21] |
| 4820 | 2755 | — | 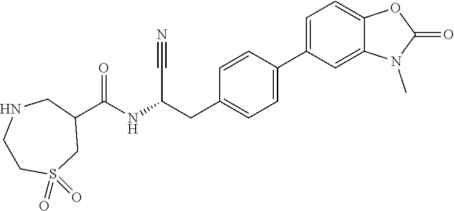 [SMILES: Cn1c(=O)oc2ccc(-c3ccc(C[C@@H](C#N)NC(=O)C4CNCCS(=O)(=O)C4)cc3)cc21] |
| >5000 | >5000 | — | 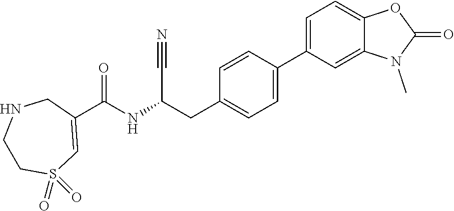 [SMILES: Cn1c(=O)oc2ccc(-c3ccc(C[C@@H](C#N)NC(=O)C4=CS(=O)(=O)CCNC4)cc3)cc21] |
| >5000 | >5000 | — | 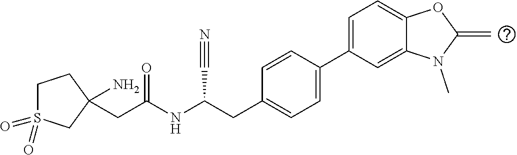 [SMILES: C=C1Oc2ccc(-c3ccc(C[C@@H](C#N)NC(=O)CC4(N)CCS(=O)(=O)C4)cc3)cc2N1C] |
| >5000 | >5000 | — | 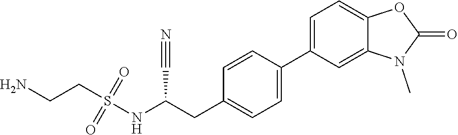 [SMILES: Cn1c(=O)oc2ccc(-c3ccc(C[C@@H](C#N)NS(=O)(=O)CCN)cc3)cc21] |
| >5000 | >5000 | — | 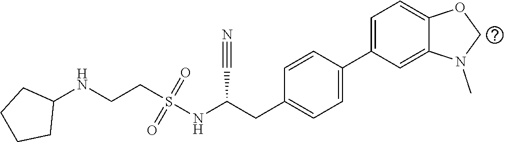 [SMILES: CN1COc2ccc(-c3ccc(C[C@@H](C#N)NS(=O)(=O)CCNC4CCCC4)cc3)cc21] |
| 1758 | 1897 | — | 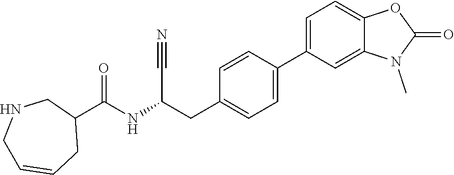 [SMILES: Cn1c(=O)oc2ccc(-c3ccc(C[C@@H](C#N)NC(=O)C4CC=CCNC4)cc3)cc21] |
| 2.3 | 2.0 | 13.4 | 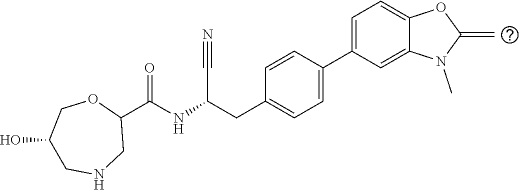 [SMILES: C=C1Oc2ccc(-c3ccc(C[C@@H](C#N)NC(=O)C4CNC[C@H](O)CO4)cc3)cc2N1C] |
| 2027 | 2174 | — | 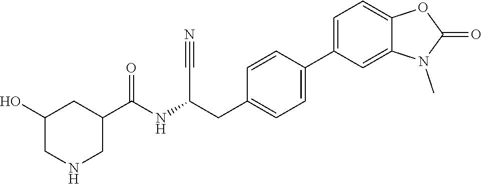 [SMILES: Cn1c(=O)oc2ccc(-c3ccc(C[C@@H](C#N)NC(=O)C4CNCC(O)C4)cc3)cc21] |
| >5000 | >5000 | — | 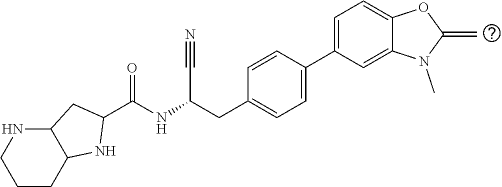 [SMILES: C=C1Oc2ccc(-c3ccc(C[C@@H](C#N)NC(=O)C4CC5NCCCC5N4)cc3)cc2N1C] |
| 784 | 438 | 878 | 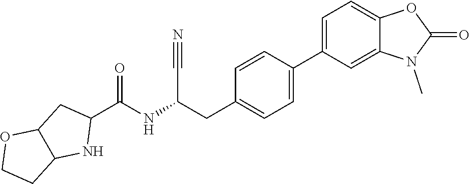 [SMILES: Cn1c(=O)oc2ccc(-c3ccc(C[C@@H](C#N)NC(=O)C4CC5OCCC5N4)cc3)cc21] |
| >5000 | >5000 | — | 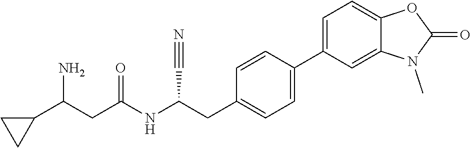 [SMILES: Cn1c(=O)oc2ccc(-c3ccc(C[C@@H](C#N)NC(=O)CC(N)C4CC4)cc3)cc21] |
| >5000 | >5000 | — | 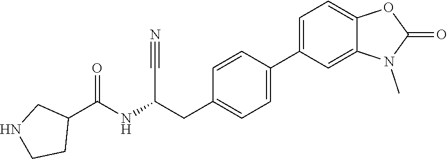 [SMILES: Cn1c(=O)oc2ccc(-c3ccc(C[C@@H](C#N)NC(=O)C4CCNC4)cc3)cc21] |
| 1099.8 | 815.2 | 40.1 | 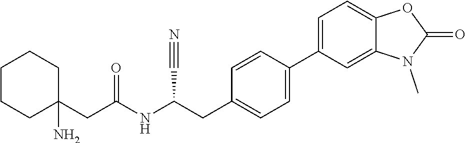 [SMILES: Cn1c(=O)oc2ccc(-c3ccc(C[C@@H](C#N)NC(=O)CC4(N)CCCCC4)cc3)cc21] |
| 828 | 2084.1 | 60.4 | 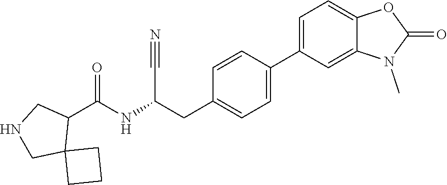 [SMILES: Cn1c(=O)oc2ccc(-c3ccc(C[C@@H](C#N)NC(=O)C4CNCC45CCC5)cc3)cc21] |
| >5000 | >5000 | — | 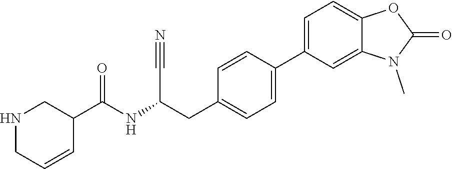 [SMILES: Cn1c(=O)oc2ccc(-c3ccc(C[C@@H](C#N)NC(=O)C4C=CCNC4)cc3)cc21] |
| >5000 | >5000 | — | 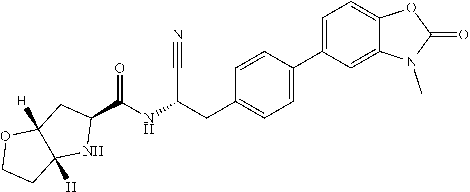 [SMILES: Cn1c(=O)oc2ccc(-c3ccc(C[C@@H](C#N)NC(=O)[C@@H]4C[C@H]5OCC[C@H]5N4)cc3)cc21] |
| 497 | 225.6 | >1000 | 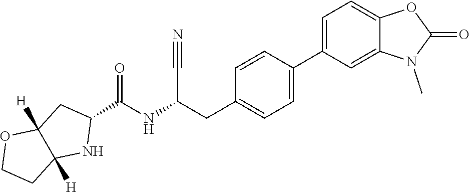 [SMILES: Cn1c(=O)oc2ccc(-c3ccc(C[C@@H](C#N)NC(=O)[C@H]4C[C@H]5OCC[C@H]5N4)cc3)cc21] |
| 744.6 | 582.6 | — | 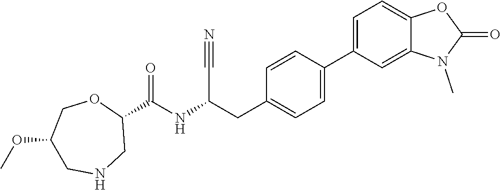 [SMILES: CO[C@H]1CNC[C@@H](C(=O)N[C@H](C#N)Cc2ccc(-c3ccc4oc(=O)n(C)c4c3)cc2)OC1] |
| 360.1 | 785.2 | 54.7 | 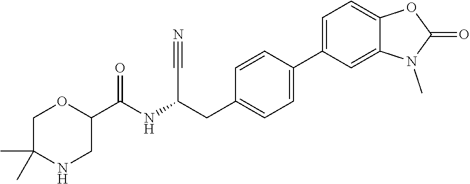 [SMILES: Cn1c(=O)oc2ccc(-c3ccc(C[C@@H](C#N)NC(=O)C4CNC(C)(C)CO4)cc3)cc21] |
| 18.6 | — | 2.1 | 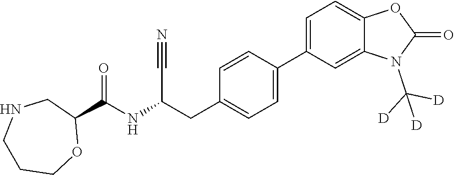 [SMILES: [2H]C([2H])([2H])n1c(=O)oc2ccc(-c3ccc(C[C@@H](C#N)NC(=O)[C@@H]4CNCCCO4)cc3)cc21] |
| 6.6 | 8.1 | 1.3 | 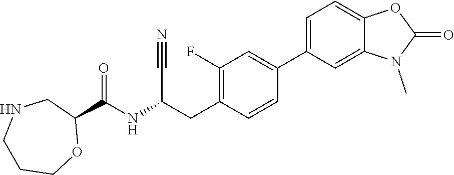 [SMILES: Cn1c(=O)oc2ccc(-c3ccc(C[C@@H](C#N)NC(=O)[C@@H]4CNCCCO4)c(F)c3)cc21] |
| 31.4 | 31 | 3.6 | 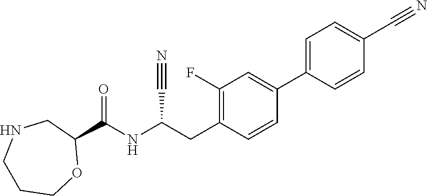 [SMILES: N#Cc1ccc(-c2ccc(C[C@@H](C#N)NC(=O)[C@@H]3CNCCCO3)c(F)c2)cc1] |
| 49.5 | 36.7 | 4.2 | 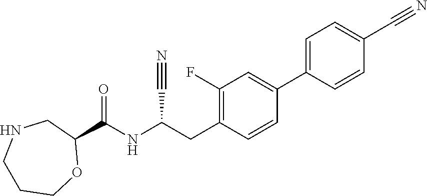 [SMILES: N#Cc1ccc(-c2ccc(C[C@@H](C#N)NC(=O)[C@@H]3CNCCCO3)c(F)c2)cc1] |
| 6 | — | 1.2 | 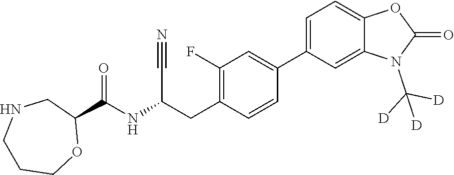 [SMILES: [2H]C([2H])([2H])n1c(=O)oc2ccc(-c3ccc(C[C@@H](C#N)NC(=O)[C@@H]4CNCCCO4)c(F)c3)cc21] |
| 1657.3 | — | — | 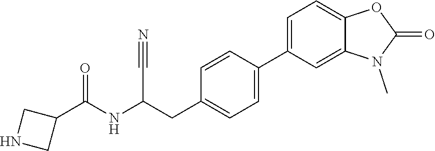 [SMILES: Cn1c(=O)oc2ccc(-c3ccc(CC(C#N)NC(=O)C4CNC4)cc3)cc21] |
| >5000 | 4349 | — | 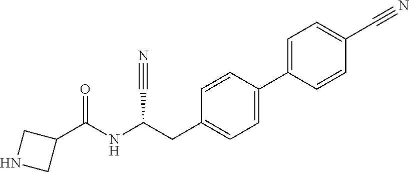 [SMILES: N#Cc1ccc(-c2ccc(C[C@@H](C#N)NC(=O)C3CNC3)cc2)cc1] |
| 1505.6 | — | — | 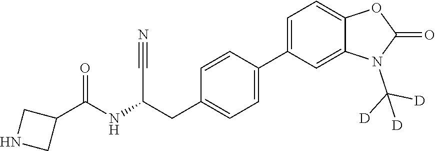 [SMILES: [2H]C([2H])([2H])n1c(=O)oc2ccc(-c3ccc(C[C@@H](C#N)NC(=O)C4CNC4)cc3)cc21] |
| >5000 | 4933.5 | — | 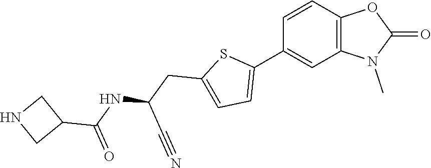 [SMILES: Cn1c(=O)oc2ccc(-c3ccc(C[C@@H](C#N)NC(=O)C4CNC4)s3)cc21] |
| >5000 | >5000 | — | 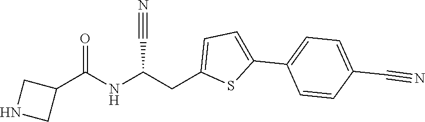 [SMILES: N#Cc1ccc(-c2ccc(C[C@@H](C#N)NC(=O)C3CNC3)s2)cc1] |
| 4940.7 | — | — | 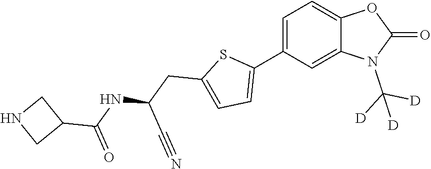 [SMILES: [2H]C([2H])([2H])n1c(=O)oc2ccc(-c3ccc(C[C@@H](C#N)NC(=O)C4CNC4)s3)cc21] |
| 431.8 | 646.6 | 507.9 | 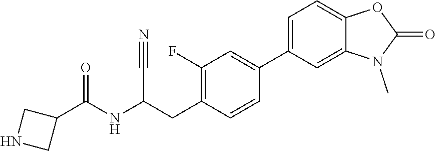 [SMILES: Cn1c(=O)oc2ccc(-c3ccc(CC(C#N)NC(=O)C4CNC4)c(F)c3)cc21] |
| 1563.8 | 1846.6 | — | 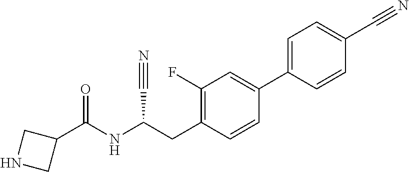 [SMILES: N#Cc1ccc(-c2ccc(C[C@@H](C#N)NC(=O)C3CNC3)c(F)c2)cc1] |
| 416.1 | — | 417.8 | 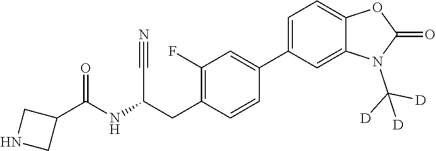 [SMILES: [2H]C([2H])([2H])n1c(=O)oc2ccc(-c3ccc(C[C@@H](C#N)NC(=O)C4CNC4)c(F)c3)cc21] |
| 311 | 135.5 | 462 | 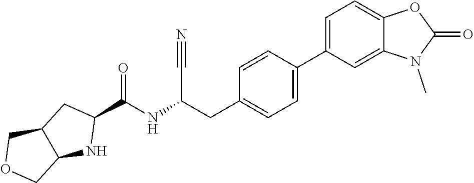 [SMILES: Cn1c(=O)oc2ccc(-c3ccc(C[C@@H](C#N)NC(=O)[C@@H]4C[C@@H]5COC[C@@H]5N4)cc3)cc21] |
| 480.3 | 245.7 | 875 | 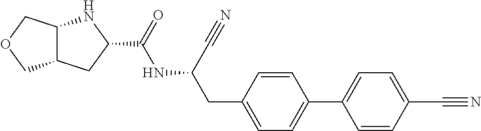 [SMILES: N#Cc1ccc(-c2ccc(C[C@@H](C#N)NC(=O)[C@@H]3C[C@@H]4COC[C@@H]4N3)cc2)cc1] |
| 425.7 | — | 500 | 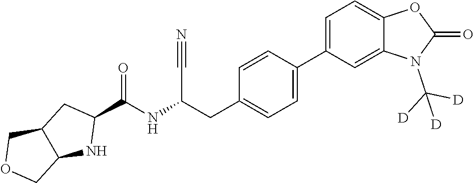 [SMILES: [2H]C([2H])([2H])n1c(=O)oc2ccc(-c3ccc(C[C@@H](C#N)NC(=O)[C@@H]4C[C@@H]5COC[C@@H]5N4)cc3)cc21] |
| 588.9 | 246.6 | — | 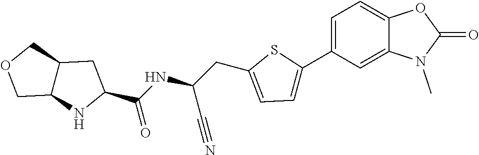 [SMILES: Cn1c(=O)oc2ccc(-c3ccc(C[C@@H](C#N)NC(=O)[C@@H]4C[C@@H]5COC[C@@H]5N4)s3)cc21] |
| 2270.5 | 365.9 | — | 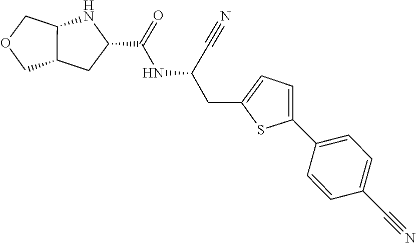 [SMILES: N#Cc1ccc(-c2ccc(C[C@@H](C#N)NC(=O)[C@@H]3C[C@@H]4COC[C@@H]4N3)s2)cc1] |
| 663.9 | — | — | 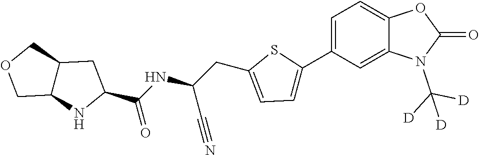 [SMILES: [2H]C([2H])([2H])n1c(=O)oc2ccc(-c3ccc(C[C@@H](C#N)NC(=O)[C@@H]4C[C@@H]5COC[C@@H]5N4)s3)cc21] |
| 178.6 | 41.2 | 150.2 | 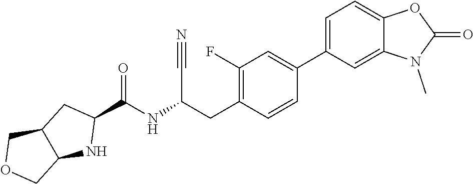 [SMILES: Cn1c(=O)oc2ccc(-c3ccc(C[C@@H](C#N)NC(=O)[C@@H]4C[C@@H]5COC[C@@H]5N4)c(F)c3)cc21] |
| 526.2 | 106.7 | — | 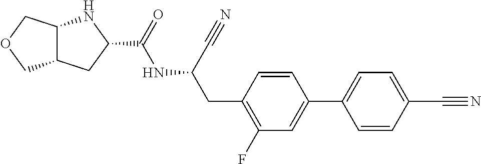 [SMILES: N#Cc1ccc(-c2ccc(C[C@@H](C#N)NC(=O)[C@@H]3C[C@@H]4COC[C@@H]4N3)c(F)c2)cc1] |
| 102.5 | — | 146 | 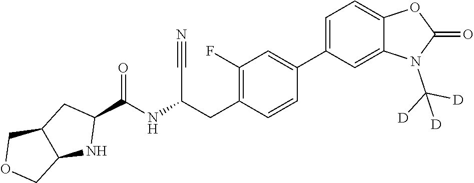 [SMILES: [2H]C([2H])([2H])n1c(=O)oc2ccc(-c3ccc(C[C@@H](C#N)NC(=O)[C@@H]4C[C@@H]5COC[C@@H]5N4)c(F)c3)cc21] |
| 186.3 | 217.9 | 10.1 | 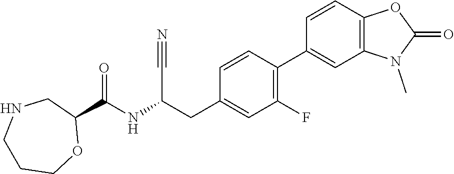 [SMILES: Cn1c(=O)oc2ccc(-c3ccc(C[C@@H](C#N)NC(=O)[C@@H]4CNCCCO4)cc3F)cc21] |
| >5000 | >5000 | — | 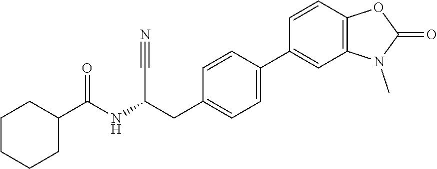 [SMILES: Cn1c(=O)oc2ccc(-c3ccc(C[C@@H](C#N)NC(=O)C4CCCCC4)cc3)cc21] |
| 10.1 | 6.5 | 3 ± 0.4 (n = 3) | 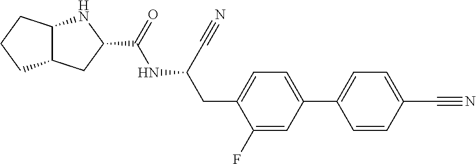 [SMILES: N#Cc1ccc(-c2ccc(C[C@@H](C#N)NC(=O)[C@@H]3C[C@@H]4CCC[C@@H]4N3)c(F)c2)cc1] |
| 5.9 | 3.4 | 1.7 | 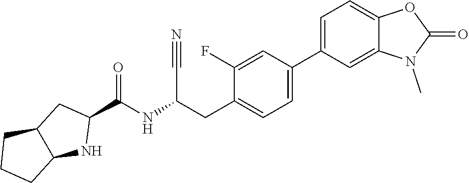 [SMILES: Cn1c(=O)oc2ccc(-c3ccc(C[C@@H](C#N)NC(=O)[C@@H]4C[C@@H]5CCC[C@@H]5N4)c(F)c3)cc21] |
| 17.3 | 8.2 | 3.1 | 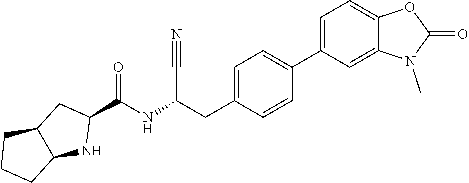 [SMILES: Cn1c(=O)oc2ccc(-c3ccc(C[C@@H](C#N)NC(=O)[C@@H]4C[C@@H]5CCC[C@@H]5N4)cc3)cc21] |
| >5000 | >5000 | — | 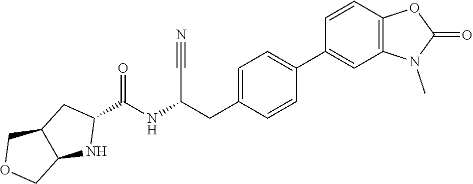 [SMILES: Cn1c(=O)oc2ccc(-c3ccc(C[C@@H](C#N)NC(=O)[C@H]4C[C@@H]5COC[C@@H]5N4)cc3)cc21] |
| >5000 | >5000 | — | 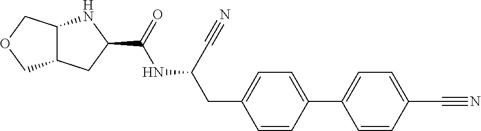 [SMILES: N#Cc1ccc(-c2ccc(C[C@@H](C#N)NC(=O)[C@H]3C[C@@H]4COC[C@@H]4N3)cc2)cc1] |
| 29.6 | — | 3.8 | 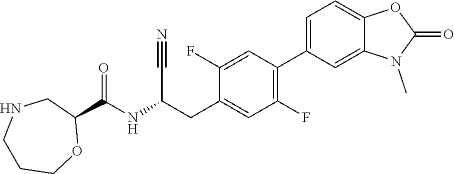 [SMILES: Cn1c(=O)oc2ccc(-c3cc(F)c(C[C@@H](C#N)NC(=O)[C@@H]4CNCCCO4)cc3F)cc21] |
| 42.2 | — | 4.9 | 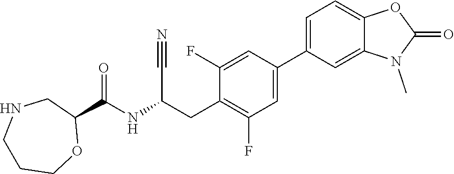 [SMILES: Cn1c(=O)oc2ccc(-c3cc(F)c(C[C@@H](C#N)NC(=O)[C@@H]4CNCCCO4)c(F)c3)cc21] |
| 428.1 | — | 59.9 | 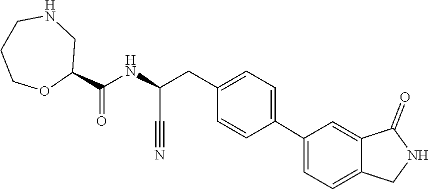 [SMILES: N#C[C@H](Cc1ccc(-c2ccc3c(c2)C(=O)NC3)cc1)NC(=O)[C@@H]1CNCCCO1] |
| 7.6 | — | 1.9 | 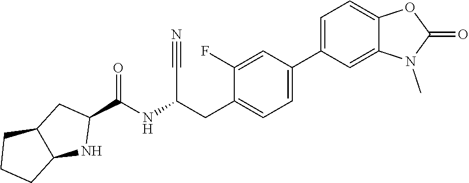 [SMILES: Cn1c(=O)oc2ccc(-c3ccc(C[C@@H](C#N)NC(=O)[C@@H]4C[C@@H]5CCC[C@@H]5N4)c(F)c3)cc21] |
| >5000 | — | — | 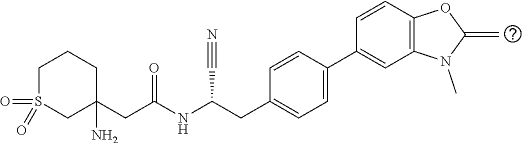 [SMILES: C=C1Oc2ccc(-c3ccc(C[C@@H](C#N)NC(=O)CC4(N)CCCS(=O)(=O)C4)cc3)cc2N1C] |
| 1217.4 | — | — | 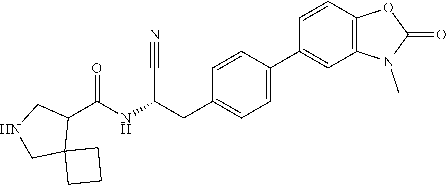 [SMILES: Cn1c(=O)oc2ccc(-c3ccc(C[C@@H](C#N)NC(=O)C4CNCC45CCC5)cc3)cc21] |
| 1324.1 | — | — | 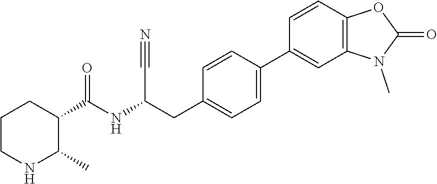 [SMILES: C[C@@H]1NCCC[C@@H]1C(=O)N[C@H](C#N)Cc1ccc(-c2ccc3oc(=O)n(C)c3c2)cc1] |
| 4475.4 | — | — | 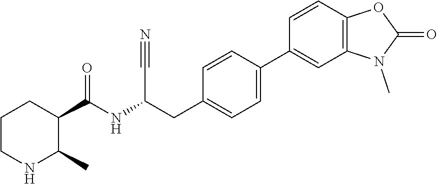 [SMILES: C[C@H]1NCCC[C@H]1C(=O)N[C@H](C#N)Cc1ccc(-c2ccc3oc(=O)n(C)c3c2)cc1] |
| 1040.5 | — | — | 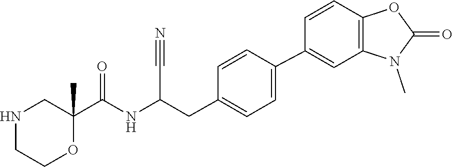 [SMILES: Cn1c(=O)oc2ccc(-c3ccc(CC(C#N)NC(=O)[C@@]4(C)CNCCO4)cc3)cc21] |
| >5000 | — | — | 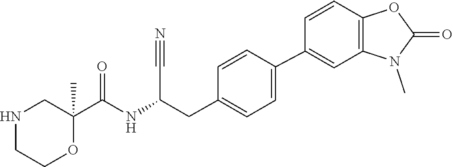 [SMILES: Cn1c(=O)oc2ccc(-c3ccc(C[C@@H](C#N)NC(=O)[C@]4(C)CNCCO4)cc3)cc21] |

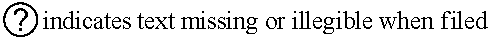

### Example 13. Synthesis of Compound 145: (S)—N—((S)-1-cyano-2-(4-(3-(methyl-d3)-2-oxo-2,3-dihydrobenzo[d]oxazol-5-yl)phenyl)ethyl)-1,4-oxazepane-2-carboxamide

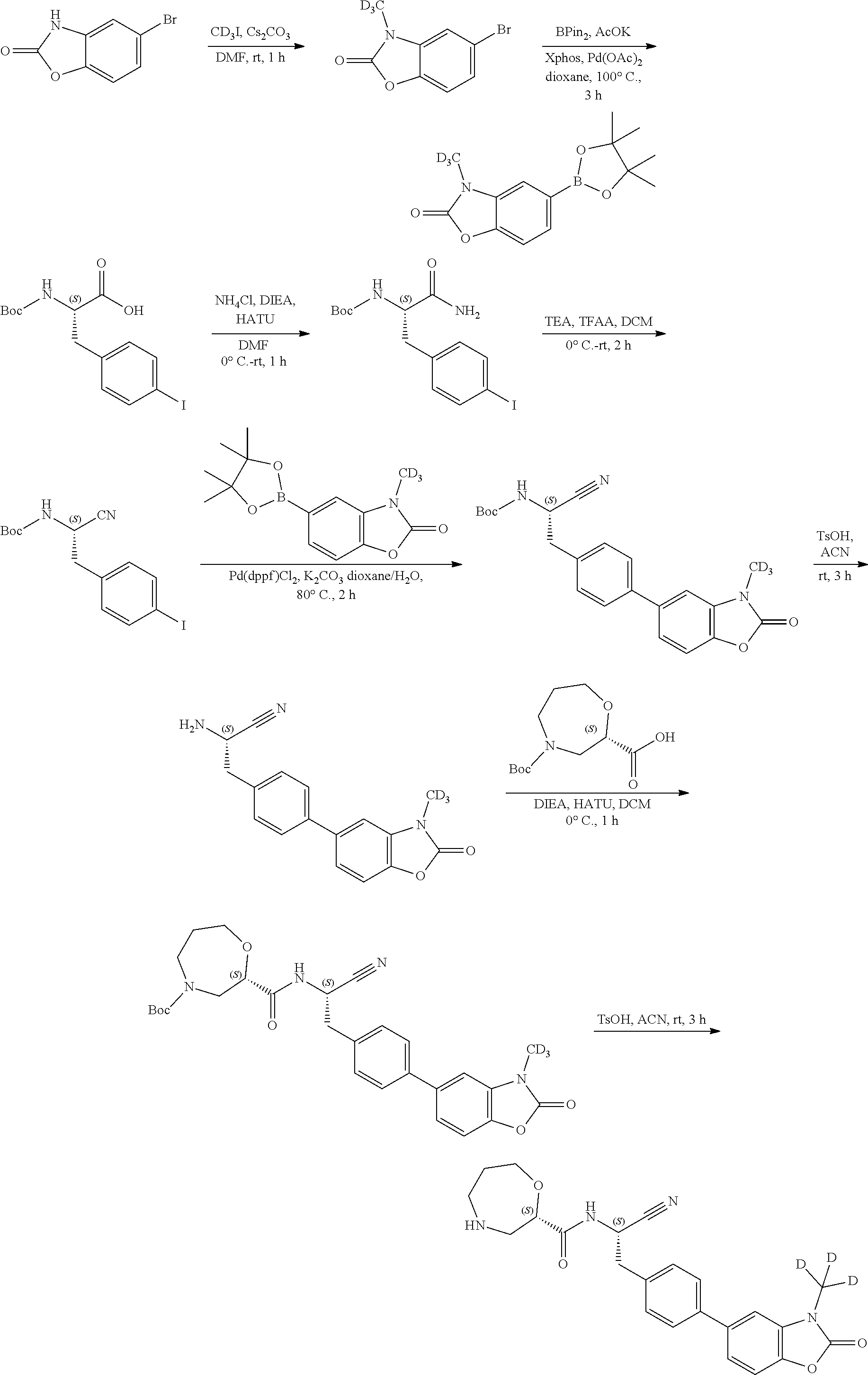

### Synthesis of 5-bromo-3-(methyl-d3)benzo[d]oxazol-2(3H)-one

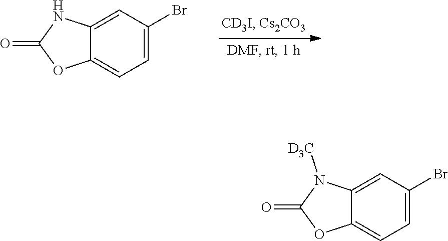 [SMILES: O=c1[nH]c2cc(Br)ccc2o1 ; [2H]C([2H])([2H])n1c(=O)oc2ccc(Br)cc21]

To a stirred mixture of 5-bromobenzo[d]oxazol-2(3H)-one (15 g, 70.08 mmol, 1.0 equiv) and Cs₂CO₃ (45.67 g, 140.17 mmol, 2.0 equiv) in DMF (150 mL) were added CD₃I (12.19 g, 84.10 mmol, 1.2 equiv) dropwise at 0° C. under nitrogen atmosphere. The resulting mixture was stirred for 1 h at room temperature. The resulting mixture was diluted with water (600 mL), extracted with EtOAc (200 mL×3). The combined organic layer was washed with brine (300 mL×2), dried over anhydrous Na₂SO₄. After filtration, the filtrate was concentrated under reduced pressure. The residue was purified by trituration with PE:EA=10:1 (100 mL). This resulted in 5-bromo-3-(methyl-d3)benzo[d]oxazol-2(3H)-one (14 g, 86.4%) as a brown solid. LCMS (ES, m/z): [M+H]⁺: 231.

### Synthesis of 3-(methyl-d3)-5-(4,4,5,5-tetramethyl-1,3,2-dioxaborolan-2-yl)benzo[d]oxazol-2(3H)-one

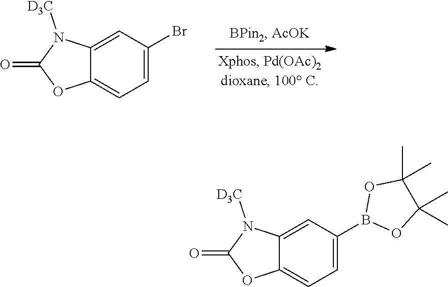 [SMILES: [2H]C([2H])([2H])n1c(=O)oc2ccc(Br)cc21 ; [2H]C([2H])([2H])n1c(=O)oc2ccc(B3OC(C)(C)C(C)(C)O3)cc21]

To a stirred solution of 5-bromo-3-(methyl-d3)benzo[d]oxazol-2(3H)-one (14 g, 60.59 mmol, 1.0 equiv) and bis(pinacolato)diboron (18.5 g, 72.70 mmol, 1.2 equiv) in dioxane (150 mL) were added AcOK (11.89 g, 121.18 mmol, 2.0 equiv) and Xphos (17.3 g, 36.35 mmol, 0.6 equiv) and Pd(OAc)₂ (2.7 g, 12.11 mmol, 0.2 equiv) in sequence. The resulting mixture was stirred for 3 h at 100° C. under nitrogen atmosphere. The mixture was allowed to cool down to room temperature. The resulting mixture was filtered, the filter cake was washed with CH₂Cl₂ (2×50 mL). The filtrate was concentrated under reduced pressure. The residue was purified by silica gel column chromatography, eluted with PE/THF (12:1) to afford 3-(methyl-d3)-5-(4,4,5,5-tetramethyl-1,3,2-dioxaborolan-2-yl)benzo[d]oxazol-2(3H)-one (13 g, 77.1%) as a brown solid.

LCMS (ES, m/z): [M+H]⁺: 279.

### Synthesis of tert-butyl N-[(1S)-1-carbamoyl-2-(4-iodophenyl)ethyl]carbamate

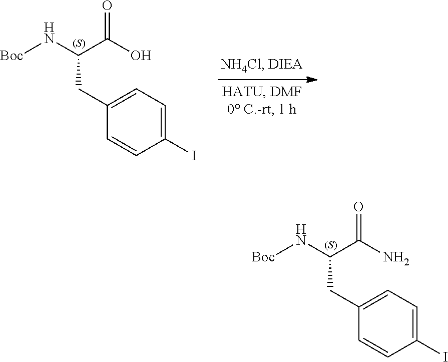

To a stirred mixture of (2S)-2-[(tert-butoxycarbonyl)amino]-3-(4-iodophenyl) propanoic acid (140 g, 357.86 mmol, 1.0 equiv) and NH₄Cl (38.3 g, 715.73 mmol, 2.0 equiv) in DMF (1.4 L) were added DIEA (138.8 g, 1073.60 mmol, 3.0 equiv) and HATU (163.3 g, 429.44 mmol, 1.2 equiv) in portions at 0° C. under nitrogen atmosphere. The resulting mixture was stirred for 1 h at room temperature under nitrogen atmosphere. The resulting mixture was diluted with water (5 L). The precipitated solid was collected by filtration and washed with water (1 L×2), dried under infrared lamp for 4 h. This resulted in tert-butyl N-[(1S)-1-carbamoyl-2-(4-iodophenyl)ethyl]carbamate (130 g, 93.0%) as a white solid. LCMS (ES, m/z): [M+H]⁺: 391.

### Synthesis of tert-butyl N-[(1S)-1-cyano-2-(4-iodophenyl)ethyl]carbamate

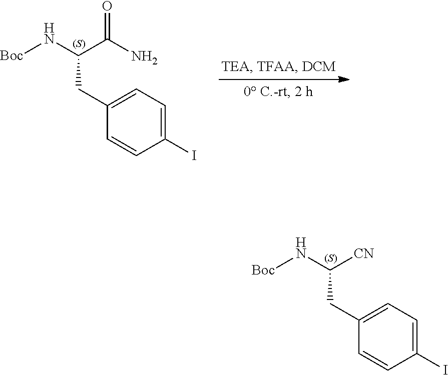

To a stirred solution of tert-butyl N-[(1S)-1-carbamoyl-2-(4-iodophenyl)ethyl]carbamate (130 g, 333.14 mmol, 1.0 equiv) and TEA (134.85 g, 1332.58 mmol, 4.0 equiv) in DCM (2 L) were added TFAA (139.94 g, 666.29 mmol, 2.0 equiv) dropwise at 0° C. under nitrogen atmosphere. The resulting mixture was stirred for 2 h at room temperature under nitrogen atmosphere. The reaction was diluted with water (1.5 L), extracted with CH₂Cl₂ (1 Lx 3). The combined organic layer was washed with brine (1 L), dried over anhydrous Na₂SO₄. After filtration, the filtrate was concentrated under reduced pressure. The residue was purified by trituration with PE:EA=20:1 (300 mL). This resulted in tert-butyl N-[(1S)-1-cyano-2-(4-iodophenyl)ethyl]carbamate (115 g, 92.7%) as a white solid. LCMS (ES, m/z): [M+H]⁺: 373.

### Synthesis of tert-butyl N-[(1S)-1-cyano-2-{4-[3-(2H3)methyl-2-oxo-1,3-benzoxazol-5-yl]phenyl}ethyl]carbamate

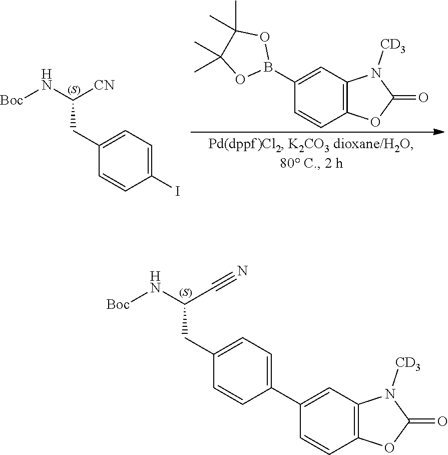

To a stirred solution of tert-butyl N-[(1S)-1-cyano-2-(4-iodophenyl)ethyl]carbamate (1.5 g, 4.03 mmol, 1.0 equiv) and 3-(2H3)methyl-5-(4,4,5,5-tetramethyl-1,3,2-dioxaborolan-2-yl)-1,3-benzoxazol-2-one (1.35 g, 4.83 mmol, 1.2 equiv) in dioxane (20 mL) and H₂O (2 mL) were added K₂CO₃ (1.11 g, 8.06 mmol, 2.0 equiv) and Pd(dppf)Cl₂ (0.29 g, 0.40 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (3:1) to afford tert-butyl N-[(1S)-1-cyano-2-{4-[3-(2H3)methyl-2-oxo-1,3-benzoxazol-5-yl]phenyl}ethyl]carbamate (1.3 g, 81.3%) as a brown solid. LCMS (ES, m/z): [M+H]⁺: 397.

### Synthesis of (2S)-2-amino-3-{4-[3-(2H3)methyl-2-oxo-1,3-benzoxazol-5-yl]phenyl}propanenitrile

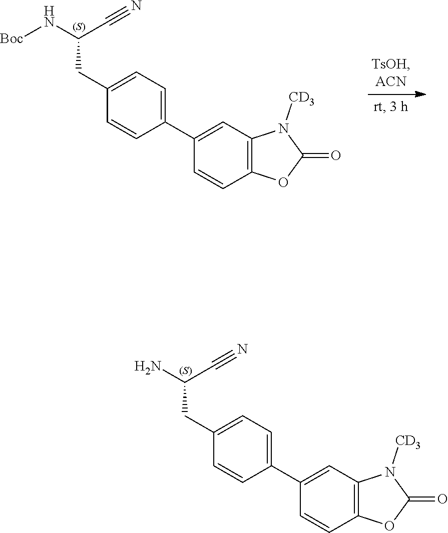

To a stirred solution of tert-butyl N-[(1S)-1-cyano-2-{4-[3-(2H3)methyl-2-oxo-1,3-benzoxazol-5-yl]phenyl}ethyl]carbamate (1.3 g, 3.27 mmol, 1.0 equiv) in ACN (13 mL) was added TsOH (1.69 g, 9.83 mmol, 3.0 equiv). The resulting mixture was stirred for 3 h at room temperature. The residue was basified to pH 8 with saturated NaHCO₃ (aq.). The resulting mixture was extracted with EtOAc (30 mL×3). The combined organic layer was washed with brine (30 mL), dried over anhydrous Na₂SO₄. After filtration, the filtrate was concentrated under reduced pressure. The residue was purified by silica gel column chromatography, eluted with PE/THF (1:1) to afford (2S)-2-amino-3-{4-[3-(2H3)methyl-2-oxo-1,3-benzoxazol-5-yl]phenyl}propanenitrile (0.75 g, 77.1%) as a white solid. LCMS (ES, m/z): [M+H]⁺: 297.

### Synthesis of tert-butyl (2S)-2-{[(1S)-1-cyano-2-{4-[3-(2H3)methyl-2-oxo-1,3-benzoxazol-5-yl]phenyl}ethyl]carbamoyl}-1,4-oxazepane-4-carboxylate

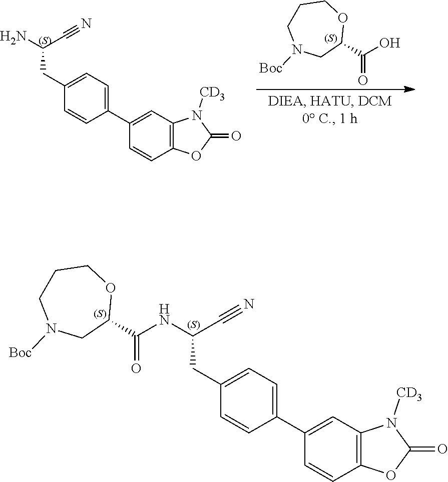

To a stirred solution of (2S)-2-amino-3-{4-[3-(2H3)methyl-2-oxo-1,3-benzoxazol-5-yl]phenyl}propanenitrile (90 mg, 0.30 mmol, 1.2 equiv) and (2S)-4-(tert-butoxycarbonyl)-1,4-oxazepane-2-carboxylic acid (51 mg, 0.21 mmol, 0.69 equiv) in DCM (2 mL) were added DIEA (98 mg, 0.75 mmol, 2.50 equiv) and HATU (115 mg, 0.30 mmol, 1.00 equiv) in portions at 0° C. under nitrogen atmosphere. The resulting mixture was stirred for 1 h at room temperature under nitrogen atmosphere. The residue was purified by silica gel column chromatography, eluted with PE/THF (2:1) to afford tert-butyl (2S)-2-{[(1S)-1-cyano-2-{4-[3-(2H3)methyl-2-oxo-1,3-benzoxazol-5-yl]phenyl}ethyl]carbamoyl}-1,4-oxazepane-4-carboxylate (120 mg, 90.5%) as a white solid. LCMS (ES, m/z): [M+H]⁺: 524.

### Synthesis of (2S)—N-[(1S)-1-cyano-2-{4-[3-(2H3)methyl-2-oxo-1,3-benzoxazol-5-yl]phenyl}ethyl]-1,4-oxazepane-2-carboxamide

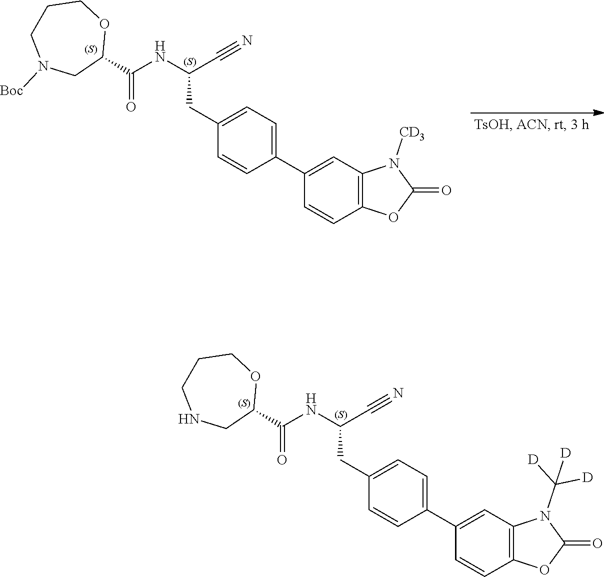

To a stirred solution of tert-butyl (2S)-2-{[(1S)-1-cyano-2-{4-[3-(2H3)methyl-2-oxo-1,3-benzoxazol-5-yl]phenyl}ethyl]carbamoyl}-1,4-oxazepane-4-carboxylate (110 mg, 0.21 mmol, 1.0 equiv) in ACN (2 mL) was added TsOH (98 mg, 0.57 mmol, 3.0 equiv). The resulting mixture was stirred for 3 h at room temperature. The crude product was purified by Prep-HPLC with the following conditions: Column, XBridge Prep C18 OBD Column, 19*150 mm 5 um; mobile phase, Water (10 mmol/L NH₄HCO₃) and ACN (30% Phase B up to 40% in 7 min); Detector, UV 254 nm. The fraction of the target was freezing dried, this resulted in (2S)—N-[(1S)-1-cyano-2-{4-[3-(2H3)methyl-2-oxo-1,3-benzoxazol-5-yl]phenyl}ethyl]-1,4-oxazepane-2-carboxamide (30 mg, 37.0%) as an off-white solid.

LCMS (ES, m/z): [M+H]⁺: 424

¹H NMR (300 MHz, DMSO-d6) δ 8.61 (d, J=8.5 Hz, 1H), 7.66 (d, J=8.1 Hz, 2H), 7.57 (s, 1H), 7.44-7.35 (m, 4H), 5.03 (q, J=8.2 Hz, 1H), 4.01 (dd, J=8.0, 3.6 Hz, 1H), 3.90-3.80 (m, 1H), 3.77-3.68 (m, 1H), 3.24-3.18 (m, 2H), 3.05 (dd, J=14.3, 3.7 Hz, 1H), 2.83-2.73 (m, 1H), 2.69-2.53 (m, 2H), 1.76-1.69 (m, 2H).

### Example 15. Synthesis of Compound 146: (S)—N—((S)-1-cyano-2-(2-fluoro-4-(3-methyl-2-oxo-2,3-dihydrobenzo[d]oxazol-5-yl)phenyl)ethyl)-1,4-oxazepane-2-carboxamide

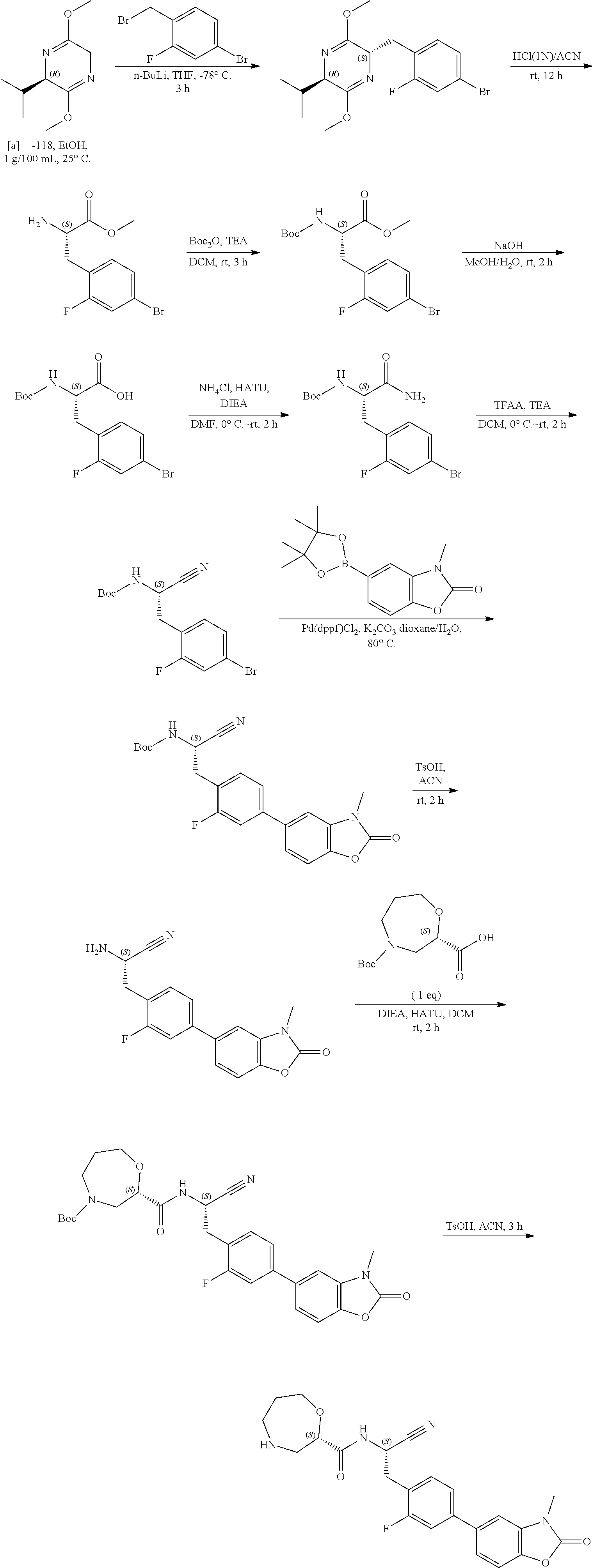

### Synthesis of (2S,5R)-2-(4-bromo-2-fluorobenzyl)-5-isopropyl-3,6-dimethoxy-2,5-dihydropyrazine

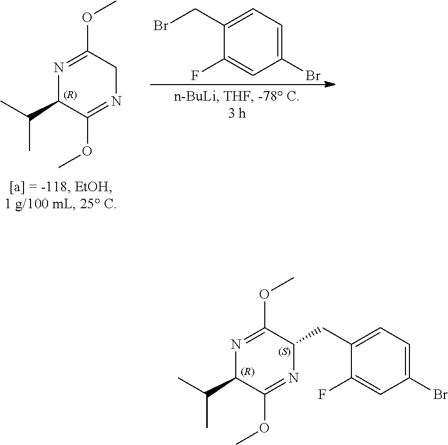

To a solution of (3R)-3-isopropyl-2,5-dimethoxy-3,6-dihydropyrazine (24.5 g, 132.98 mmol, 1.0 equiv) in THF (500 mL), n-BuLi (2.5 M in hexane) (63.8 mL, 159.58 mmol, 1.2 equiv) was added dropwise at −78° C. under nitrogen gas atmosphere. After addition, the clear solution was stirred for 1 h at the same temperature, before 4-bromo-1-(bromomethyl)-2-fluorobenzene (39.2 g, 146.28 mmol, 1.1 equiv) in THF (50 mL) was added at −78° C. and stirred for additional 2 h. The reaction was quenched with NH₄Cl(aq) (500 mL), extracted with ethyl acetate (400 mL×2). The combined organic phase was washed with brine (300 mL), dried over anhydrous sodium sulfate. Filtered and the filtrate was concentrated under reduced pressure, the residue was purified by silica gel column with ethyl acetate/petroleum ether (5%). The fraction of the target was concentrated, (2S,5R)-2-(4-bromo-2-fluorobenzyl)-5-isopropyl-3,6-dimethoxy-2,5-dihydropyrazine (40 g, 81%) was obtained as colorless oil. LCMS (ES, m/z): [M+H]⁺: 371.

### Synthesis of methyl (2S)-2-amino-3-(4-bromo-2-fluorophenyl) propanoate

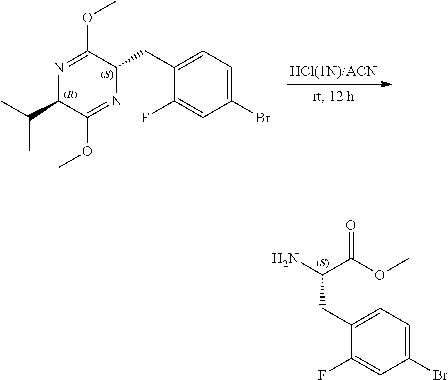

To a stirred solution of (2S,5R)-2-(4-bromo-2-fluorobenzyl)-5-isopropyl-3,6-dimethoxy-2,5-dihydropyrazine (37 g, 99.66 mmol, 1.0 equiv) in ACN (330 mL) was added HCl (1 N) (150 mL, 150.00 mmol, 1.5 equiv) dropwise at room temperature. The resulting mixture was stirred for 12 h at room temperature. Concentrated to remove the solvent, the residue was acidified to pH 8 with saturated NaHCO₃ (aq.), extracted with dichloromethane (300 mL×3). The combined organic layer was dried over anhydrous Na₂SO₄. After filtration, the filtrate was concentrated under reduced pressure. This resulted in methyl (2S)-2-amino-3-(4-bromo-2-fluorophenyl) propanoate (23 g, 83%) as a light yellow oil and used to the next step without further purification. LCMS (ES, m/z): [M+H]⁺: 276.

### Synthesis of methyl (2S)-3-(4-bromo-2-fluorophenyl)-2-[(tert-butoxycarbonyl)amino]propanoate

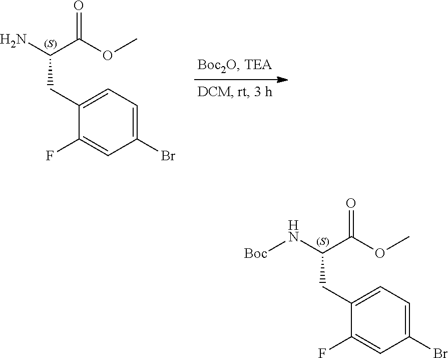

To a stirred mixture of methyl (2S)-2-amino-3-(4-bromo-2-fluorophenyl) propanoate (23.0 g, 83.30 mmol, 1.0 equiv) and TEA (23.16 mL, 166.60 mmol, 2.0 equiv) in DCM (350 mL) was added Boc₂O (21.8 g, 99.96 mmol, 1.2 equiv) at room temperature. The resulting mixture was stirred for 3 h at room temperature. The resulting mixture were washed with brine (200 mL×2), dried over anhydrous Na₂SO₄. After filtration, the filtrate was concentrated under reduced pressure. The residue was purified by silica gel column chromatography, eluted with PE 1 THF (2:1) to afford methyl (2S)-3-(4-bromo-2-fluorophenyl)-2-[(tert-butoxycarbonyl)amino]propanoate (22 g, 70%) as a light yellow oil. LCMS (ES, m/z): [M+H]⁺: 376.

### Synthesis of (2S)-3-(4-bromo-2-fluorophenyl)-2-[(tert-butoxycarbonyl)amino]propanoic acid

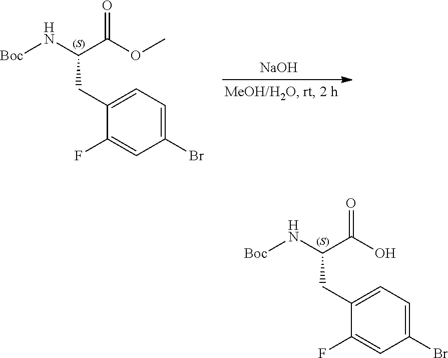

To a stirred solution of methyl (2S)-3-(4-bromo-2-fluorophenyl)-2-[(tert-butoxycarbonyl)amino]propanoate (22.0 g, 58.47 mmol, 1.0 equiv) in MeOH (250 mL) was added NaOH (4.7 g, 116.95 mmol, 2.0 equiv) in H₂O (150 mL) dropwise at room temperature. The resulting mixture was stirred for 2 h at room temperature. The resulting mixture was concentrated under reduced pressure to remove the solvent, the residue was acidified to pH 5 with citric acid, extracted with ethyl acetate (200 mL×3). The combined organic layer was dried over anhydrous Na₂SO₄. After filtration, the filtrate was concentrated under reduced pressure. This resulted in (2S)-3-(4-bromo-2-fluorophenyl)-2-[(tert-butoxycarbonyl)amino]propanoic acid (20 g, 94%) as a light yellow oil. LCMS (ES, m/z): [M+H]⁺: 362.

### Synthesis of tert-butyl N-[(1S)-2-(4-bromo-2-fluorophenyl)-1-carbamoylethyl]carbamate

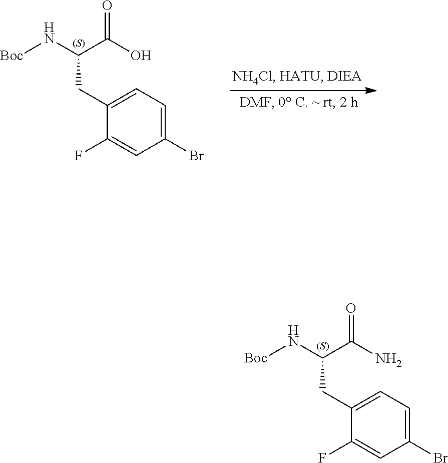

To a stirred mixture of (2S)-3-(4-bromo-2-fluorophenyl)-2-[(tert-butoxycarbonyl)amino]propanoic acid (20 g, 55.22 mmol, 1.0 equiv) and NH₄Cl (5.91 g, 110.44 mmol, 2 equiv), DIEA (21.4 g, 165.66 mmol, 3 equiv) in DMF (200 mL) was added HATU (25.2 g, 66.26 mmol, 1.2 equiv) at 0° C. The resulting mixture was stirred for 2 h at 0° C. The reaction was quenched with water (300 mL) at 0° C., extracted with EtOAc (300 mL×3). The combined organic layer was washed with brine (300 mL×3), dried over anhydrous Na₂SO₄. After filtration, the filtrate was concentrated under reduced pressure. The residue was purified by silica gel column chromatography, eluted with PE/THF (1:1) to afford tert-butyl N-[(1S)-2-(4-bromo-2-fluorophenyl)-1-carbamoylethyl]carbamate (19 g, 95%) as a light yellow oil. LCMS (ES, m/z): [M+H]⁺: 361.

### Synthesis of tert-butyl N-[(1S)-2-(4-bromo-2-fluorophenyl)-1-cyanoethyl]carbamate

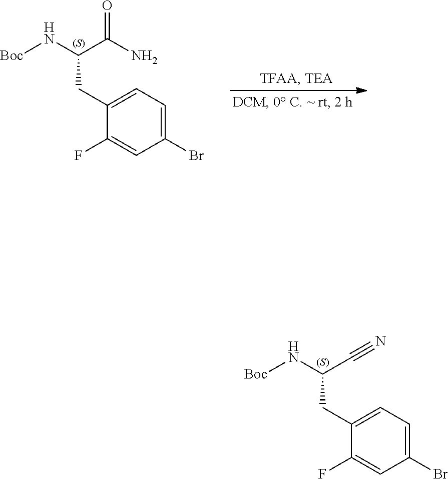

To a stirred mixture of tert-butyl N-[(1S)-2-(4-bromo-2-fluorophenyl)-1-carbamoylethyl]carbamate (10 g, 27.69 mmol, 1.0 equiv) and TEA (11.2 g, 110.74 mmol, 4.0 equiv) in DCM (100 mL) was added TFAA (8.7 g, 41.53 mmol, 1.5 equiv) dropwise at 0° C. The resulting mixture was stirred for 2 h at room temperature. The resulting mixture were washed with brine (100 mL×3), dried over anhydrous Na₂SO₄. After filtration, the filtrate was concentrated under reduced pressure. The residue was purified by silica gel column chromatography, eluted with PE/THF (3:1) to afford tert-butyl N-[(1S)-2-(4-bromo-2-fluorophenyl)-1-cyanoethyl]carbamate (7.5 g, 79%) as a light yellow oil. LCMS (ES, m/z): [M+H]⁺: 343.

### Synthesis of tert-butyl (S)-(1-cyano-2-(2-fluoro-4-(3-methyl-2-oxo-2,3-dihydrobenzo[d]oxazol-5-yl)phenyl)ethyl) carbamate

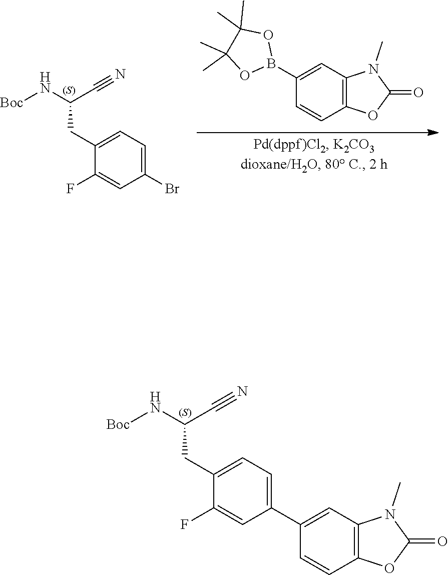

To a stirred mixture of tert-butyl N-[(1S)-2-(4-bromo-2-fluorophenyl)-1-cyanoethyl]carbamate (1.0 g, 2.91 mmol, 1.0 equiv), 3-methyl-5-(4,4,5,5-tetramethyl-1,3,2-dioxaborolan-2-yl)-1,3-benzoxazol-2-one (0.88 g, 3.20 mmol, 1.1 equiv) in dioxane (10 mL) and H₂O (1 mL), K₂CO₃ (0.8 g, 5.82 mmol, 2.0 equiv) and Pd(dppf)Cl₂ (0.2 g, 0.29 mmol, 0.1 equiv) were added at room temperature. The resulting mixture was stirred for 2 h at 80° C. under nitrogen atmosphere. The reaction was cooled to room temperature, concentrated to remove the solvent, the residue was purified by silica gel column chromatography, eluted with PE/THF (4:1) to afford tert-butyl (S)-(1-cyano-2-(2-fluoro-4-(3-methyl-2-oxo-2,3-dihydrobenzo[d]oxazol-5-yl)phenyl)ethyl) carbamate (1.1 g, 91%) as a yellow oil. LCMS (ES, m/z): [M+H]⁺: 412.

### Synthesis of (S)-2-amino-3-(2-fluoro-4-(3-methyl-2-oxo-2,3-dihydrobenzo[d]oxazol-5-yl)phenyl)propanenitrile

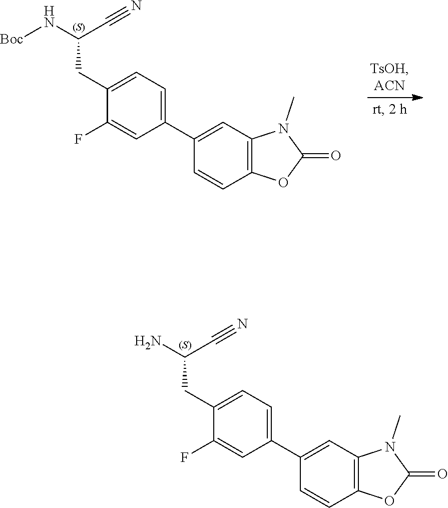

Into a 100 mL round-bottom flask were added tert-butyl (S)-(1-cyano-2-(2-fluoro-4-(3-methyl-2-oxo-2,3-dihydrobenzo[d]oxazol-5-yl)phenyl)ethyl) carbamate (1.0 g, 2.43 mmol, 1.0 equiv), acetonitrile (30 mL) and TsOH (1.3 g, 7.29 mmol, 3.0 equiv) in sequence at room temperature. The resulting mixture was stirred for 2 h at room temperature. The PH value of the mixture was basified to 8 with saturated NaHCO₃ (aq.), extracted with ethyl acetate (100 mL*3). The combined organic layer was washed with brine (100 mL×2), dried over anhydrous Na₂SO₄. After filtration, the filtrate was concentrated under reduced pressure. This resulted in (S)-2-amino-3-(2-fluoro-4-(3-methyl-2-oxo-2,3-dihydrobenzo[d]oxazol-5-yl)phenyl)propanenitrile (1.0 g, crude) as a white solid and used to the next step without further purification. LCMS (ES, m/z): [M+H]⁺: 312.

### Synthesis of tert-butyl (S)-2-(((S)-1-cyano-2-(2-fluoro-4-(3-methyl-2-oxo-2,3-dihydrobenzo[d]oxazol-5-yl)phenyl)ethyl) carbamoyl)-1,4-oxazepane-4-carboxylate

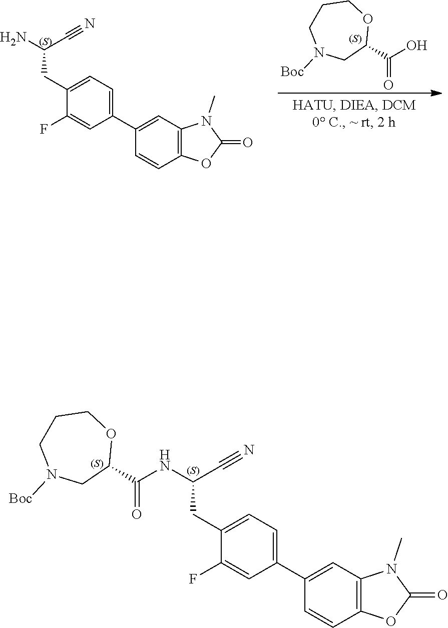

To a stirred solution of (2S)-4-(tert-butoxycarbonyl)-1,4-oxazepane-2-carboxylic acid (50 mg, 0.20 mmol, 1.0 equiv), (S)-2-amino-3-(2-fluoro-4-(3-methyl-2-oxo-2,3-dihydrobenzo[d]oxazol-5-yl)phenyl)propanenitrile (76 mg, 0.24 mmol, 1.2 equiv) and DIEA (79 mg, 0.61 mmol, 3.0 equiv) in DCM (5 mL) was added HATU (93 mg, 0.24 mmol, 1.2 equiv) in portions at 0° C. The resulting mixture was stirred for 2 h at room temperature. The residue was purified by silica gel column chromatography, eluted with PE/THF (1:1) to afford tert-butyl (S)-2-(((S)-1-cyano-2-(2-fluoro-4-(3-methyl-2-oxo-2,3-dihydrobenzo[d]oxazol-5-yl)phenyl)ethyl) carbamoyl)-1,4-oxazepane-4-carboxylate (70 mg, 64%) as a white solid. LCMS (ES, m/z): [M+H]⁺: 539.

### Synthesis of (S)—N—((S)-1-cyano-2-(2-fluoro-4-(3-methyl-2-oxo-2,3-dihydrobenzo[d]oxazol-5-yl)phenyl)ethyl)-1,4-oxazepane-2-carboxamide

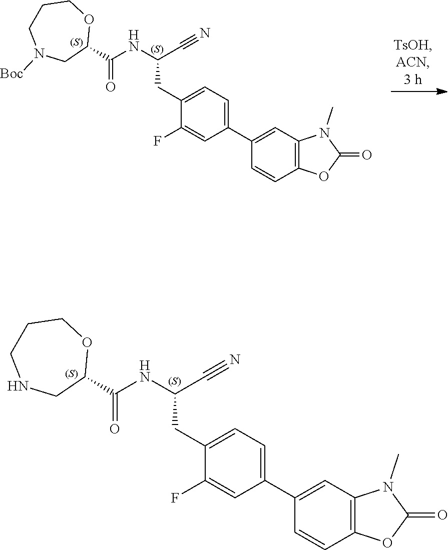

Into a 8 mL vial were added tert-butyl (S)-2-(((S)-1-cyano-2-(2-fluoro-4-(3-methyl-2-oxo-2,3-dihydrobenzo[d]oxazol-5-yl)phenyl)ethyl) carbamoyl)-1,4-oxazepane-4-carboxylate (70 mg, 0.13 mmol, 1.0 equiv), ACN (3 mL) and TsOH (67 mg, 0.39 mmol, 3.0 equiv) in sequence at room temperature. The resulting mixture was stirred for 3 h at room temperature. The reaction solution was filtered and the filtrate was purified by reversed-phase flash chromatography with the following conditions: C18-120 g column, mobile phase, MeCN in Water (0.1% NH₃·H₂O), 10% to 80% gradient in 10 min; detector, UV 254 nm. The fraction of the target was freezing dried, this resulted in (S)—N—((S)-1-cyano-2-(2-fluoro-4-(3-methyl-2-oxo-2,3-dihydrobenzo[d]oxazol-5-yl)phenyl)ethyl)-1,4-oxazepane-2-carboxamide (22.6 mg, 40%) as a white solid.

### Analytical Data

LCMS (ES, m/z): [M+H]⁺: 439.1

¹H NMR (400 MHz, DMSO-d₆) δ 8.70 (d, J=8.4 Hz, 1H), 7.66 (d, J=2.0 Hz, 1H), 7.63-7.52 (m, 2H), 7.52-7.42 (m, 2H), 7.41 (d, J=8.4 Hz, 1H), 5.06 (q, J=8.4 Hz, 1H), 4.01 (dd, J=8.0, 3.6 Hz, 1H), 3.90-3.84 (m, 1H), 3.77-3.70 (m, 1H), 3.41 (s, 3H), 3.31-3.17 (m, 1H), 3.06 (dd, J=14.2, 3.7 Hz, 1H), 2.83-2.73 (m, 1H), 2.68-2.54 (m, 2H), 1.83-1.64 (m, 2H).

### Example 16. Synthesis of compound 147: (2S)—N-[(1S)-1-cyano-2-{4′-cyano-3-fluoro-[1,1′-biphenyl]-4-yl}ethyl]-1,4-oxazepane-2-carboxamide

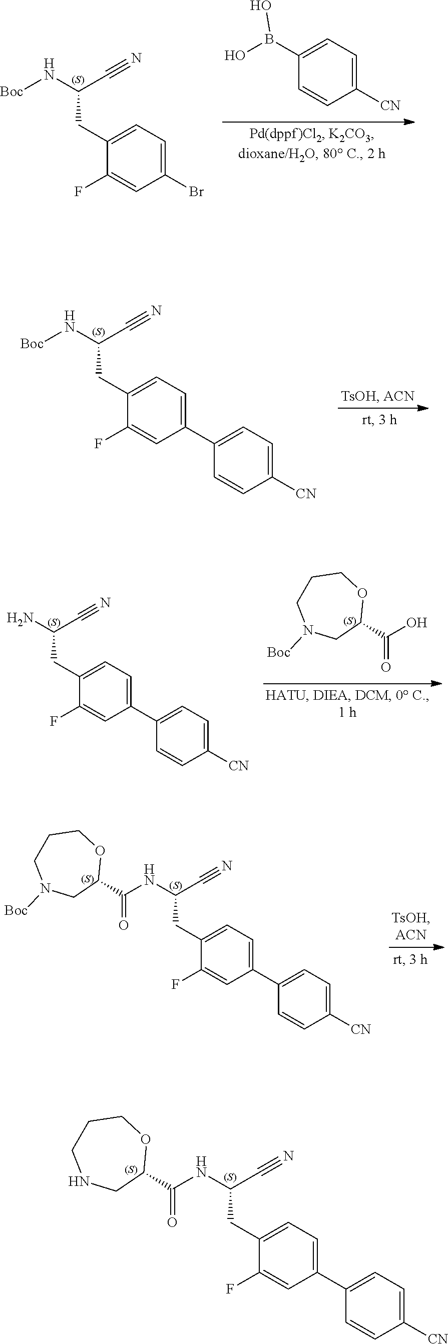

### Synthesis of tert-butyl N-[(1S)-1-cyano-2-{4′-cyano-3-fluoro-[1,1′-biphenyl]-4-yl}ethyl]carbamate

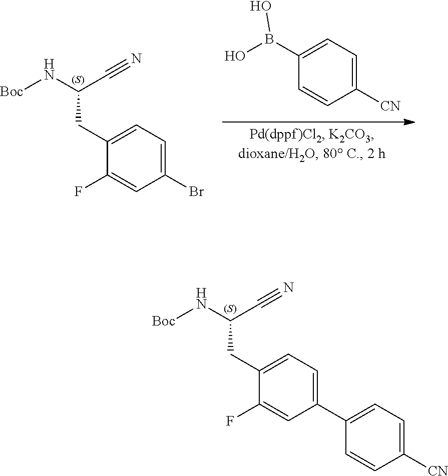

To a stirred solution of tert-butyl N-[(1S)-2-(4-bromo-2-fluorophenyl)-1-cyanoethyl]carbamate (1.0 g, 2.91 mmol, 1.0 equiv) and 4-cyanophenylboronic acid (0.51 g, 3.49 mmol, 1.2 equiv) in dioxane (10 mL) and H₂O (1 mL) were added K₂CO₃ (0.81 g, 5.83 mmol, 2.0 equiv) and Pd(dppf)Cl₂ (0.21 g, 0.29 mmol, 0.1 equiv) in sequence. The resulting mixture was stirred for 2 h at 80° C. under nitrogen atmosphere. The reaction was cooled to room temperature, The resulting mixture was concentrated under reduced pressure. The residue was purified by silica gel column chromatography, eluted with PE/EA (2:1) to afford tert-butyl N-[(1S)-1-cyano-2-{4′-cyano-3-fluoro-[1,1′-biphenyl]-4-yl}ethyl]carbamate (1.0 g, 93%) as a light yellow oil. LCMS (ES) [M+1]⁺ m/z: 366.

### Synthesis of 4′-[(2S)-2-amino-2-cyanoethyl]-3′-fluoro-[1,1′-biphenyl]-4-carbonitrile

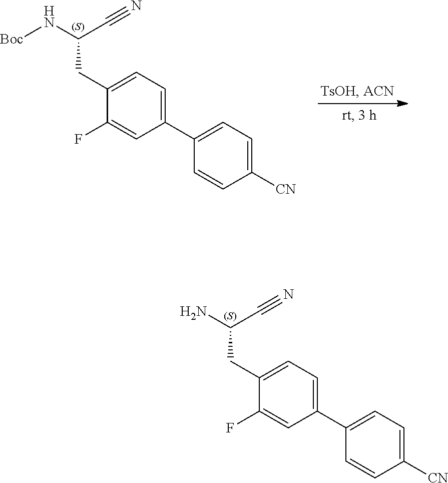

To a stirred solution of tert-butyl N-[(1S)-1-cyano-2-{4′-cyano-3-fluoro-[1,1′-biphenyl]-4-yl}ethyl]carbamate (1.0 g, 2.74 mmol, 1.0 equiv) in ACN (12 mL) was added TsOH (1.41 g, 8.21 mmol, 3.0 equiv). The resulting mixture was stirred for 3 h at room temperature. The mixture was basified to pH 8 with saturated NaHCO₃ (aq.), extracted with EtOAc (3×20 mL). The combined organic layer was washed with brine (30 mL), dried over anhydrous Na₂SO₄. After filtration, the filtrate was concentrated under reduced pressure. The residue was purified by silica gel column chromatography, eluted with PE/THF (1:1) to afford 4′-[(2S)-2-amino-2-cyanoethyl]-3′-fluoro-[1,1′-biphenyl]-4-carbonitrile (600 mg, 82%) as a white solid. LCMS (ES) [M+1]⁺ m/z: 266.

### Synthesis of tert-butyl (2S)-2-{[(1S)-1-cyano-2-{4′-cyano-3-fluoro-[1,1′-biphenyl]-4-yl}ethyl]carbamoyl}-1,4-oxazepane-4-carboxylate

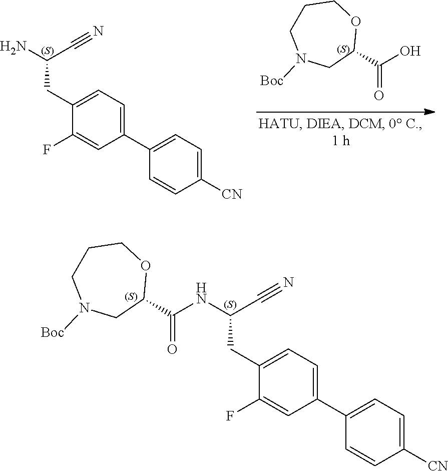

To a stirred solution of 4′-[(2S)-2-amino-2-cyanoethyl]-3′-fluoro-[1,1′-biphenyl]-4-carbonitrile (64 mg, 0.25 mmol, 1.2 equiv) and (2S)-4-(tert-butoxycarbonyl)-1,4-oxazepane-2-carboxylic acid (50 mg, 0.20 mmol, 1.0 equiv) in DCM (2 mL) was added DIEA (79 mg, 0.61 mmol, 3.0 equiv). To the above mixture was added HATU (93 mg, 0.25 mmol, 1.2 equiv) in portions at 0° C. The resulting mixture was stirred for additional 1 h at 0° C. The resulting mixture was concentrated under reduced pressure to remove the solvent. The residue was purified by silica gel column chromatography, eluted with PE/THF (2:1) to afford tert-butyl (2S)-2-{[(1S)-1-cyano-2-{4′-cyano-3-fluoro-[1,1′-biphenyl]-4-yl}ethyl]carbamoyl}-1,4-oxazepane-4-carboxylate (90 mg, 89%) as off-white solid. LCMS (ES) [M+1]⁺ m/z: 493.

### Synthesis of (2S)—N-[(1S)-1-cyano-2-{4′-cyano-3-fluoro-[1,1′-biphenyl]-4-yl}ethyl]-1,4-oxazepane-2-carboxamide

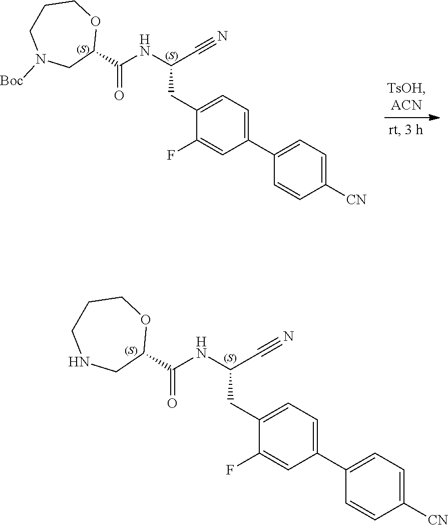

To a stirred solution of tert-butyl (2S)-2-{[(1S)-1-cyano-2-{4′-cyano-3-fluoro-[1,1′-biphenyl]-4-yl}ethyl]carbamoyl}-1,4-oxazepane-4-carboxylate (90 mg, 0.18 mmol, 1.0 equiv) in ACN (2 mL) was added TsOH (94 mg, 0.55 mmol, 3.0 equiv). The resulting mixture was stirred for 3 h at room temperature. The reaction solution was purified by Prep-HPLC with the following conditions: Column, XBridge Prep C18 OBD Column, 19*150 mm, 5 um, mobile phase, Water (10 mmol/L NH₃·H₂O) and ACN (30% Phase B up to 40% in 7 min), Detector, UV, 220 nm. The fraction of the target was freezing dried to afford (2S)—N-[(1S)-1-cyano-2-{4′-cyano-3-fluoro-[1,1′-biphenyl]-4-yl}ethyl]-1,4-oxazepane-2-carboxamide (30 mg, 42%) as a white solid.

¹H NMR (300 MHz, DMSO-d₆) δ 8.71 (d, J=8.6 Hz, 1H), 7.94 (s, 4H), 7.72-7.57 (m, 2H), 7.51 (t, J=7.9 Hz, 1H), 5.14-5.00 (m, 1H), 4.00 (dd, J=7.9, 3.6 Hz, 1H), 3.93-3.80 (m, 1H), 3.77-3.69 (m, 1H), 3.32-3.16 (m, 2H), 3.04 (dd, J=14.2, 3.7 Hz, 1H), 2.84-2.72 (m, 1H), 2.68-2.51 (m, 2H), 1.84-1.65 (m, 2H). LCMS (ES) [M+1]⁺ m/z: 393

### Example 17. Synthesis of Compound 148: (S)—N—((S)-1-cyano-2-(2-fluoro-4-(3-(methyl-d3)-2-oxo-2,3-dihydrobenzo[d]oxazol-5-yl)phenyl)ethyl)-1,4-oxazepane-2-carboxamide

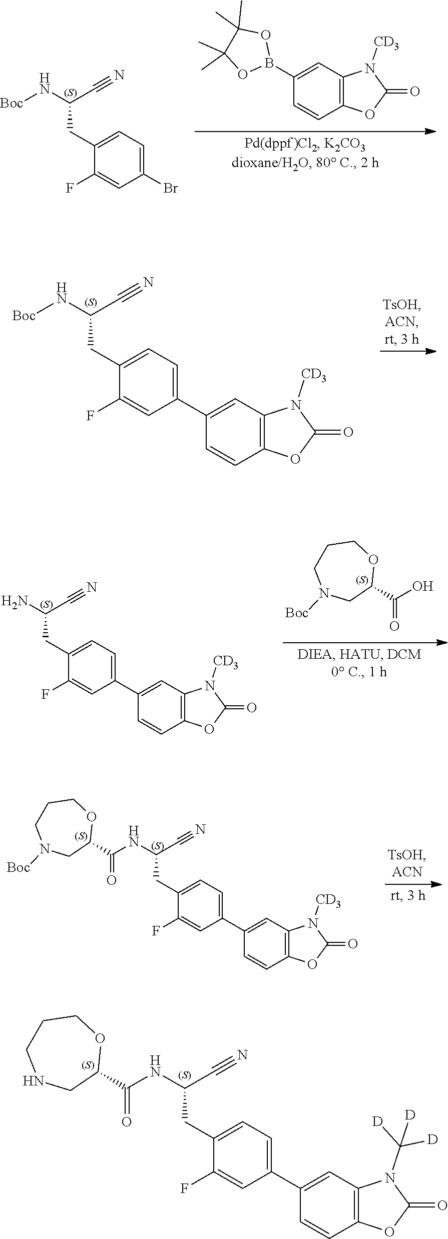

### Synthesis of tert-butyl (S)-(1-cyano-2-(2-fluoro-4-(3-(methyl-d3)-2-oxo-2,3-dihydrobenzo[d]oxazol-5-yl)phenyl)ethyl) carbamate

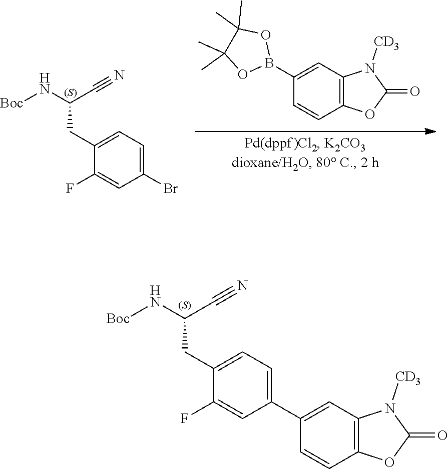

To a stirred solution of tert-butyl N-[(1S)-2-(4-bromo-2-fluorophenyl)-1-cyanoethyl]carbamate (1 g, 2.91 mmol, 1.0 equiv) and 3-(2H3)methyl-5-(4,4,5,5-tetramethyl-1,3,2-dioxaborolan-2-yl)-1,3-benzoxazol-2-one (0.97 g, 3.49 mmol, 1.2 equiv) in dioxane (10 mL) and H₂O (1 mL) were added K₂CO₃ (0.81 g, 5.82 mmol, 2.0 equiv) and Pd (dppf) C12 (0.21 g, 0.29 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (3:1) to afford tert-butyl (S)-(1-cyano-2-(2-fluoro-4-(3-(methyl-d3)-2-oxo-2,3-dihydrobenzo[d]oxazol-5-yl)phenyl)ethyl) carbamate (1 g, 83%) as light yellow solid. LCMS (ES) [M+1]⁺ m/z: 415.

### Synthesis of (S)-2-amino-3-(2-fluoro-4-(3-(methyl-d3)-2-oxo-2,3-dihydrobenzo[d]oxazol-5-yl)phenyl)propanenitrile

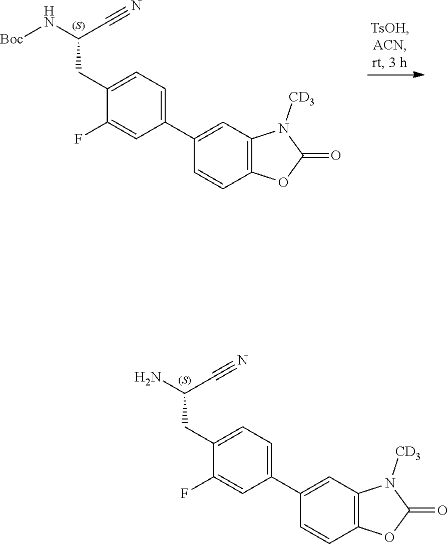

To a stirred solution of tert-butyl (S)-(1-cyano-2-(2-fluoro-4-(3-(methyl-d3)-2-oxo-2,3-dihydrobenzo[d]oxazol-5-yl)phenyl)ethyl) carbamate (1 g, 2.41 mmol, 1.0 equiv) in ACN (10 mL) was added TsOH (1.25 g, 7.23 mmol, 3.0 equiv). The resulting mixture was stirred for 3 h at room temperature. The residue was basified to pH 8 with saturated NaHCO₃ (aq.). The resulting mixture was extracted with EtOAc (3×30 mL). The combined organic layer was washed with brine (20 mL), dried over anhydrous Na₂SO₄. After filtration, the filtrate was concentrated under reduced pressure. The residue was purified by silica gel column chromatography, eluted with PE/THF (1:1) to afford(S)-2-amino-3-(2-fluoro-4-(3-(methyl-d3)-2-oxo-2,3-dihydrobenzo[d]oxazol-5-yl)phenyl)propanenitrile (0.65 g, 85.7%) as off-white solid. LCMS (ES) [M+1]⁺ m/z: 315.

### Synthesis of tert-butyl (S)-2-(((S)-1-cyano-2-(2-fluoro-4-(3-(methyl-d3)-2-oxo-2,3-dihydrobenzo[d]oxazol-5-yl)phenyl)ethyl) carbamoyl)-1,4-oxazepane-4-carboxylate

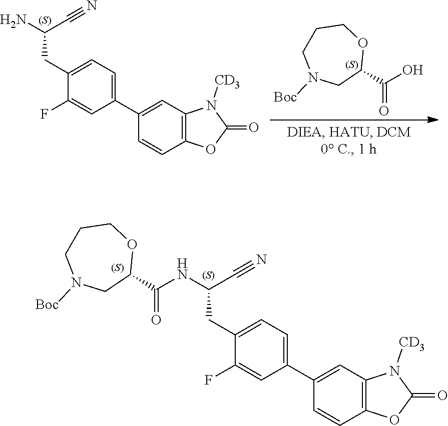

To a stirred solution of (S)-2-amino-3-(2-fluoro-4-(3-(methyl-d3)-2-oxo-2,3-dihydrobenzo[d]oxazol-5-yl)phenyl)propanenitrile (100 mg, 0.31 mmol, 1.2 equiv) and (2S)-4-(tert-butoxycarbonyl)-1,4-oxazepane-2-carboxylic acid (65 mg, 0.26 mmol, 1.0 equiv) in DCM (1 mL) were added DIEA (102 mg, 0.79 mmol, 3.0 equiv) and HATU (120 mg, 0.31 mmol, 1.2 equiv) in portions at 0° C. under nitrogen atmosphere. The resulting mixture was stirred for 1 h at 0° C. The residue was purified by silica gel column chromatography, eluted with PE/THF (2:1) to afford tert-butyl (S)-2-(((S)-1-cyano-2-(2-fluoro-4-(3-(methyl-d3)-2-oxo-2,3-dihydrobenzo[d]oxazol-5-yl)phenyl)ethyl) carbamoyl)-1,4-oxazepane-4-carboxylate (120 mg, 83.5%) as white solid. LCMS (ES) [M+1]⁺ m/z: 542.

### Synthesis of (S)—N—((S)-1-cyano-2-(2-fluoro-4-(3-(methyl-d3)-2-oxo-2,3-dihydrobenzo[d]oxazol-5-yl)phenyl)ethyl)-1,4-oxazepane-2-carboxamide

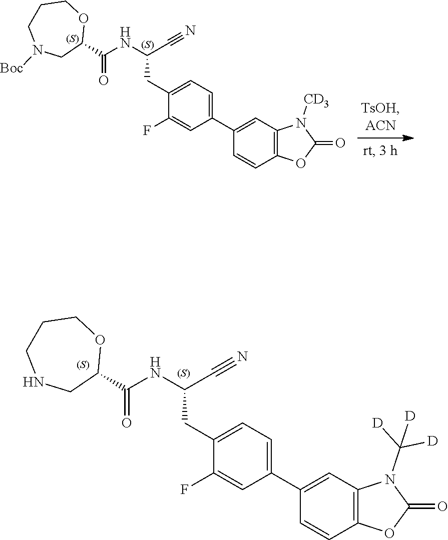

To a stirred solution of tert-butyl (S)-2-(((S)-1-cyano-2-(2-fluoro-4-(3-(methyl-d3)-2-oxo-2,3-dihydrobenzo[d]oxazol-5-yl)phenyl)ethyl) carbamoyl)-1,4-oxazepane-4-carboxylate (120 mg, 0.22 mmol, 1.0 equiv) in ACN (2 mL) was added TsOH (114 mg, 0.66 mmol, 3.0 equiv). The resulting mixture was stirred for 3 h at room temperature. The reaction solution was purified by Prep-HPLC with the following conditions XBridge Prep C18 OBD Column, 19*150 mm, 5 um; mobile phase, Water (10 mmol/L NH₄HCO₃) and ACN (30% Phase B up to 40% in 7 min); Detector, UV 254 nm. This resulted in (S)—N—((S)-1-cyano-2-(2-fluoro-4-(3-(methyl-d3)-2-oxo-2,3-dihydrobenzo[d]oxazol-5-yl)phenyl)ethyl)-1,4-oxazepane-2-carboxamide (60 mg, 61%) as white solid.

¹H NMR (300 MHz, DMSO-d₆) δ 8.70 (d, J=8.6 Hz, 1H), 7.66 (d, J=1.8 Hz, 1H), 7.61-7.54 (m, 2H), 7.52-7.36 (m, 3H), 5.06 (q, J=8.2 Hz, 1H), 4.00 (dd, J=7.8, 3.6 Hz, 1H), 3.91-3.83 (m, 1H), 3.80-3.66 (m, 1H), 3.29-3.13 (m, 2H), 3.05 (dd, J=14.2, 3.6 Hz, 1H), 2.82-2.74 (m, 1H), 2.66-2.56 (m, 2H), 1.83-1.65 (m, 2H).

LCMS (ES) [M+1]+ m/z: 442.

### Example 18. Synthesis of Compound 149: (S)—N-(1-cyano-2-(4-(3-methyl-2-oxo-2,3-dihydrobenzo[d]oxazol-5-yl)phenyl)ethyl)azetidine-3-carboxamide 2,2,2-trifluoroacetate

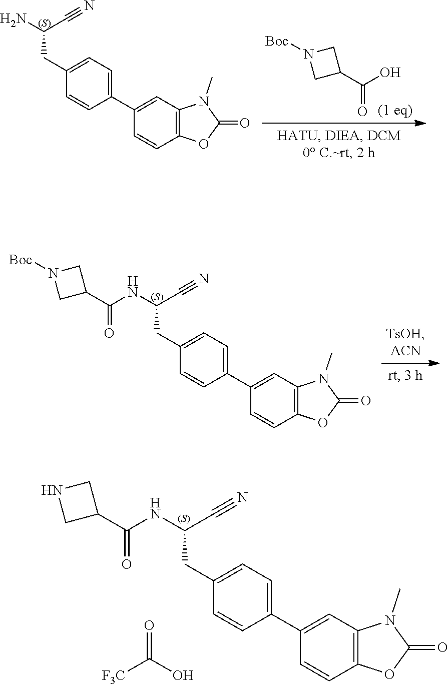

### Synthesis of tert-butyl 3-{[(1S)-1-cyano-2-[4-(3-methyl-2-oxo-1,3-benzoxazol-5-yl)phenyl]ethyl]carbamoyl}azetidine-1-carboxylate

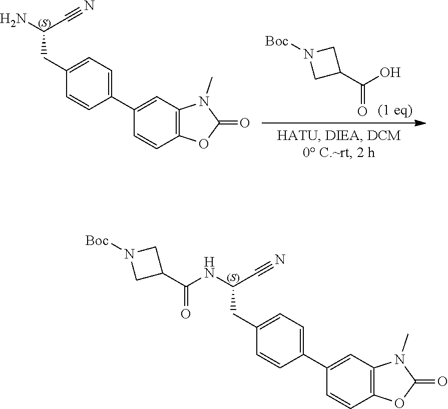

A solution of 1-(tert-butoxycarbonyl) azetidine-3-carboxylic acid (60 mg, 0.29 mmol, 1.0 equiv) in DCM (5 mL) was treated with (2S)-2-amino-3-[4-(3-methyl-2-oxo-1,3-benzoxazol-5-yl)phenyl]propanenitrile (105 mg, 0.35 mmol, 1.2 equiv), DIEA (116 mg, 0.89 mmol, 3.0 equiv. This was followed by the addition of HATU (136 mg, 0.35 mmol, 1.2 equiv) in DCM (2 mL) dropwise at 0° C. The resulting mixture was stirred for additional 2 h at room temperature. Concentrated under reduced pressure, the residue was purified by silica gel column chromatography, eluted with PE/THF (1:1) to afford tert-butyl 3-{[(1S)-1-cyano-2-[4-(3-methyl-2-oxo-1,3-benzoxazol-5-yl)phenyl]ethyl]carbamoyl}azetidine-1-carboxylate (86 mg, 60%) as white solid. LCMS (ES, m/z): [M+H]⁺: 477.

### Synthesis of (S)—N-(1-cyano-2-(4-(3-methyl-2-oxo-2,3-dihydrobenzo[d]oxazol-5-yl)phenyl)ethyl)azetidine-3-carboxamide 2,2,2-trifluoroacetate

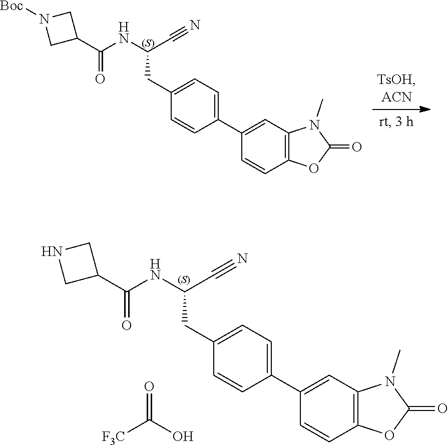

Into a 8 mL vial were added tert-butyl 3-{[(1S)-1-cyano-2-[4-(3-methyl-2-oxo-1,3-benzoxazol-5-yl)phenyl]ethyl]carbamoyl}azetidine-1-carboxylate (100 mg, 0.21 mmol, 1.0 equiv), ACN (3 mL) and TsOH (108 mg, 0.63 mmol, 3.0 equiv) in sequence at room temperature. The resulting mixture was stirred for additional 2 h at room temperature. The reaction solution was purified by reversed-phase flash chromatography with the following conditions: column, C18-120 g, mobile phase, MeCN in Water (0.1% TFA), 10% to 50% gradient in 10 min; detector, UV 254 nm. This resulted in (S)—N-(1-cyano-2-(4-(3-methyl-2-oxo-2,3-dihydrobenzo[d]oxazol-5-yl)phenyl)ethyl)azetidine-3-carboxamide 2,2,2-trifluoroacetate (24.7 mg, 24%) as white solid.

### Analytical Data

LCMS (ES, m/z): [M−TFA+H]⁺: 377

¹H NMR (400 MHz, DMSO-d₆) δ 9.10-9.00 (m, 1H), 8.65 (brs, 2H), 7.69 (d, J=8.1 Hz, 2H), 7.58 (d, J=1.6 Hz, 1H), 7.47-7.37 (m, 4H), 5.05 (q, J=7.5 Hz, 1H), 4.06-3.96 (m, 3H), 3.83-3.78 (m, 1H), 3.60-3.50 (m, 1H), 3.41 (s, 3H), 3.20-3.08 (m, 2H).

### Example 19. Synthesis of Compound 150: N-[(1S)-1-cyano-2-{4′-cyano-[1,1′-biphenyl]-4-yl}ethyl]azetidine-3-carboxamide

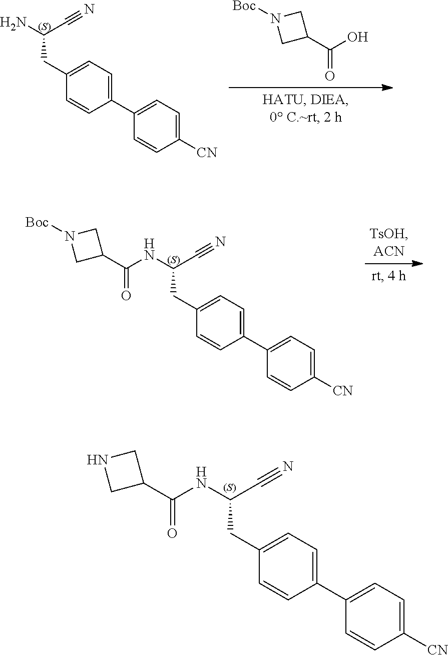

### Synthesis of tert-butyl 3-{[(1S)-1-cyano-2-{4′-cyano-[1,1′-biphenyl]-4-yl}ethyl]carbamoyl}azetidine-1-carboxylate

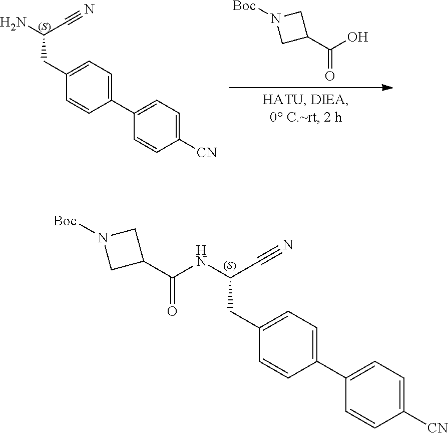

To a stirred mixture of 4′-[(2S)-2-amino-2-cyanoethyl]-[1,1′-biphenyl]-4-carbonitrile (75 mg, 0.30 mmol, 1.0 equiv) and 1-(tert-butoxycarbonyl) azetidine-3-carboxylic acid (61 mg, 0.30 mmol, 1 equiv), DIEA (117 mg, 0.91 mmol, 3.0 equiv) in DCM (5 mL) was added HATU (138 mg, 0.36 mmol, 1.2 equiv) at 0° C. The resulting mixture was stirred for 2 h at room temperature. Concentrated under reduced pressure, the residue was purified by silica gel column chromatography, eluted with PE/THF (2:1) to afford tert-butyl 3-{[(1S)-1-cyano-2-{4′-cyano-[1,1′-biphenyl]-4-yl}ethyl]carbamoyl}azetidine-1-carboxylate (90 mg, 69%) as a light yellow solid. LCMS (ES, m/z): [M+H]⁺: 431.

### Synthesis of N-[(1S)-1-cyano-2-{4′-cyano-[1,1′-biphenyl]-4-yl}ethyl]azetidine-3-carboxamide

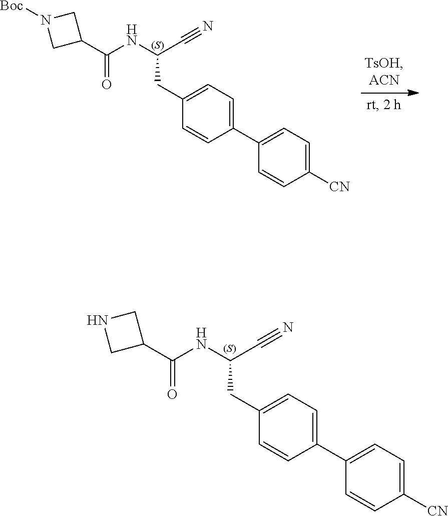

Into a 50 mL round-bottom flask were added tert-butyl 3-{[(1S)-1-cyano-2-{4′-cyano-[1,1′-biphenyl]-4-yl}ethyl]carbamoyl}azetidine-1-carboxylate (90 mg, 0.21 mmol, 1.0 equiv), ACN (3 mL) and TsOH·H₂O (119 mg, 0.63 mmol, 3.0 equiv) at room temperature. The resulting mixture was stirred for 2 h at room temperature. The reaction solution was purified by Prep-HPLC with the following conditions: Column, Atlantis Prep T3 OBD Column, 19*150 mm, 5 um; mobile phase, Water (0.05% NH₃·H₂O) and ACN (25% Phase B up to 50% in 10 min) to afford N-[(1S)-1-cyano-2-{4′-cyano-[1,1′-biphenyl]-4-yl}ethyl]azetidine-3-carboxamide (21.3 mg, 31%) as a white solid.

### Analytical Data

LCMS (ES, m/z): [M+H]⁺: 331.

¹H NMR (400 MHz, DMSO-d₆) δ 8.66 (d, J=7.9 Hz, 1H), 7.96-7.86 (m, 4H), 7.73 (d, J=7.9 Hz, 2H), 7.44 (d, J=8.0 Hz, 2H), 5.01 (q, J=7.8 Hz, 1H), 3.61 (t, J=7.0 Hz, 1H), 3.50 (t, J=7.0 Hz, 1H), 3.45-3.35 (m, 3H), 3.20-3.08 (m, 2H).

### Example 20. Synthesis of Compound 152: (S)—N-(1-cyano-2-(5-(3-methyl-2-oxo-2,3-dihydrobenzo[d]oxazol-5-yl)thiophen-2-yl)ethyl)azetidine-3-carboxamide 2,2,2-trifluoroacetate

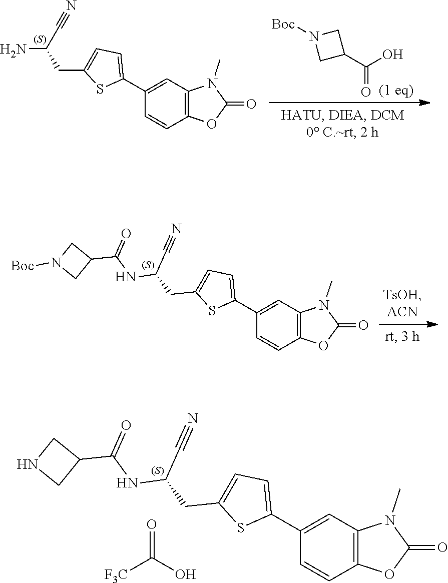

### Synthesis of tert-butyl 3-{[(1S)-1-cyano-2-[5-(3-methyl-2-oxo-1,3-benzoxazol-5-yl)thiophen-2-yl]ethyl]carbamoyl}azetidine-1-carboxylate

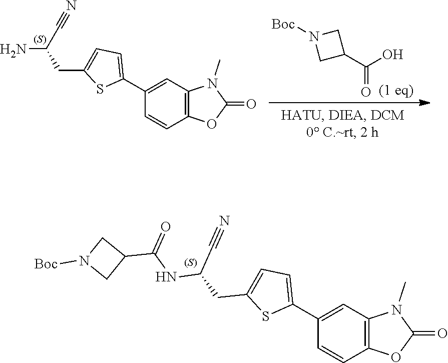

To a stirred solution of 1-(tert-butoxycarbonyl) azetidine-3-carboxylic acid (50 mg, 0.24 mmol, 1.0 equiv), (2S)-2-amino-3-[5-(3-methyl-2-oxo-1,3-benzoxazol-5-yl)thiophen-2-yl]propanenitrile (89 mg, 0.29 mmol, 1.2 equiv) and DIEA (96 mg, 0.74 mmol, 3.0 equiv) in DCM (5 mL) was added HATU (113 mg, 0.29 mmol, 1.2 equiv) in portions at 0° C. The resulting mixture was stirred for additional 2 h at room temperature. Concentrated under reduced pressure to remove the solvent, the residue was purified by silica gel column chromatography, eluted with PE/THF (1:1) to afford tert-butyl 3-{[(1S)-1-cyano-2-[5-(3-methyl-2-oxo-1,3-benzoxazol-5-yl)thiophen-2-yl]ethyl]carbamoyl}azetidine-1-carboxylate (80 mg, 66.7%) as white solid. LCMS (ES, m/z): [M+H]⁺: 483.

### Synthesis of (S)—N-(1-cyano-2-(5-(3-methyl-2-oxo-2,3-dihydrobenzo[d]oxazol-5-yl)thiophen-2-yl)ethyl)azetidine-3-carboxamide 2,2,2-trifluoroacetate

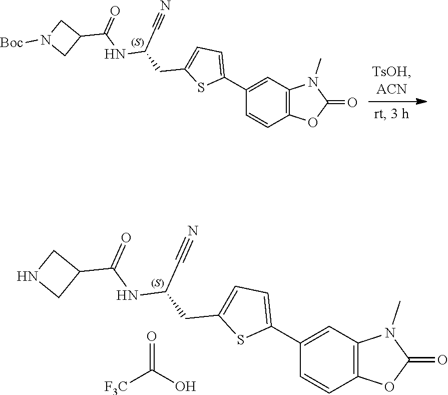

Into a 50 mL round-bottom flask were added tert-butyl 3-{[(1S)-1-cyano-2-[5-(3-methyl-2-oxo-1,3-benzoxazol-5-yl)thiophen-2-yl]ethyl]carbamoyl}azetidine-1-carboxylate (80 mg, 0.16 mmol, 1.0 equiv), TsOH (86 mg, 0.49 mmol, 3.0 equiv) and ACN (3 mL) at room temperature. The resulting mixture was stirred for additional 2 h at room temperature. The residue was purified by reversed-phase flash chromatography with the following conditions: column, C18-120 g, mobile phase, MeCN in Water (0.1% TFA), 10% to 80% gradient in 10 min; detector, UV 254 nm. The fraction of the target was freezing dried, this resulted in (S)—N-(1-cyano-2-(5-(3-methyl-2-oxo-2,3-dihydrobenzo[d]oxazol-5-yl)thiophen-2-yl)ethyl)azetidine-3-carboxamide 2,2,2-trifluoroacetate (20.5 mg, 25%) as white solid.

### Analytical Data

LCMS (ES, m/z): [M−TFA+H]⁺: 383.1

¹H NMR (400 MHz, DMSO-d₆) δ 9.13 (d, J=7.6 Hz, 1H), 8.74 (brs, 2H), 7.52 (s, 1H), 7.41 (d, J=3.6 Hz, 1H), 7.37-7.32 (m, 2H), 7.05 (d, J=3.6 Hz, 1H), 5.04 (q, J=7.4 Hz, 1H), 4.08-3.99 (m, 3H), 3.93-3.89 (m, 1H), 3.64-3.56 (m, 1H), 3.42-3.37 (m, 4H), 3.29-3.26 (m, 1H).

### Example 21. Synthesis of Compound 153: N-[(1S)-1-cyano-2-[5-(4-cyanophenyl)thiophen-2-yl]ethyl]azetidine-3-carboxamide trifluoroacetic acid

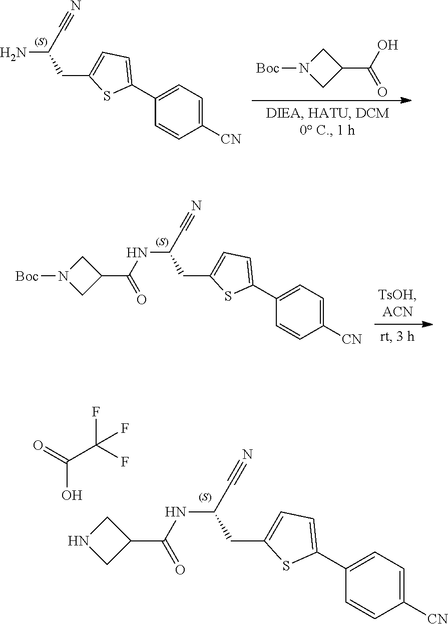

### Synthesis of tert-butyl 3-{[(1S)-1-cyano-2-[5-(4-cyanophenyl)thiophen-2-yl]ethyl]carbamoyl}azetidine-1-carboxylate

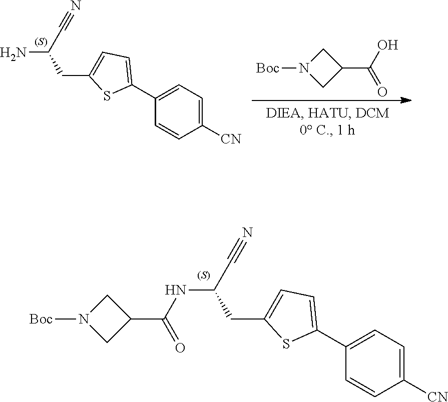

To a stirred solution of 4-{5-[(2S)-2-amino-2-cyanoethyl]thiophen-2-yl}benzonitrile (120 mg, 0.47 mmol, 1.2 equiv) and 1-(tert-butoxycarbonyl) azetidine-3-carboxylic acid (79 mg, 0.39 mmol, 1.0 equiv) in DCM (2 mL) were added DIEA (153 mg, 1.18 mmol, 3.0 equiv) and HATU (180 mg, 0.47 mmol, 1.2 equiv) in portions at 0° C. under nitrogen atmosphere. The resulting mixture was stirred for 1 h at 0° C. under nitrogen atmosphere. The residue was purified by silica gel column chromatography, eluted with PE/THF (2:1) to afford tert-butyl 3-{[(1S)-1-cyano-2-[5-(4-cyanophenyl)thiophen-2-yl]ethyl]carbamoyl}azetidine-1-carboxylate (140 mg, 81%) as white solid. LCMS (ES) [M+1]⁺ m/z: 437.

### Synthesis of N-[(1S)-1-cyano-2-[5-(4-cyanophenyl)thiophen-2-yl]ethyl]azetidine-3-carboxamide trifluoroacetic acid

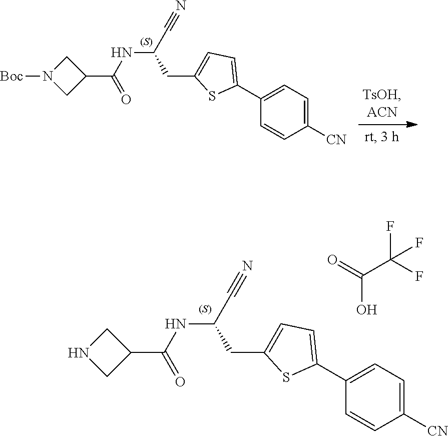

To a stirred solution of tert-butyl 3-{[(1S)-1-cyano-2-[5-(4-cyanophenyl)thiophen-2-yl]ethyl]carbamoyl}azetidine-1-carboxylate (80 mg, 0.18 mmol, 1.0 equiv) in ACN (3 mL) was added TsOH (94 mg, 0.54 mmol, 3.0 equiv). The resulting mixture was stirred for 3 h at room temperature. The reaction solution was purified by Prep-HPLC with the following conditions: Kinetex EVO C18 Column, 21.2*150, 5 um, mobile phase, Water (0.1% TFA) and ACN (10% Phase B up to 50% in 15 min); Detector, UV 254 nm. The fraction of the target was freezing dried, this resulted in N-[(1S)-1-cyano-2-[5-(4-cyanophenyl)thiophen-2-yl]ethyl]azetidine-3-carboxamide trifluoroacetic acid (20 mg, 24%) as white solid.

¹H NMR (300 MHz, DMSO-d₆) δ 9.14 (d, J=7.7 Hz, 1H), 8.77 (brs, 2H), 7.89-7.79 (m, 4H), 7.63 (d, J=3.7 Hz, 1H), 7.11 (d, J=3.7 Hz, 1H), 5.06 (q, J=7.8 Hz, 1H), 4.12-3.95 (m, 3H), 3.89 (dd, J=10.5, 7.2 Hz, 1H), 3.68-3.51 (m, 1H), 3.47-3.34 (m, 2H).

LCMS (ES) [M+1]⁺ m/z: 337.

### Example 22. Synthesis of Compound 154: (S)—N-(1-cyano-2-(5-(3-(methyl-d3)-2-oxo-2,3-dihydrobenzo[d]oxazol-5-yl)thiophen-2-yl)ethyl)azetidine-3-carboxamide 2,2,2-trifluoroacetate

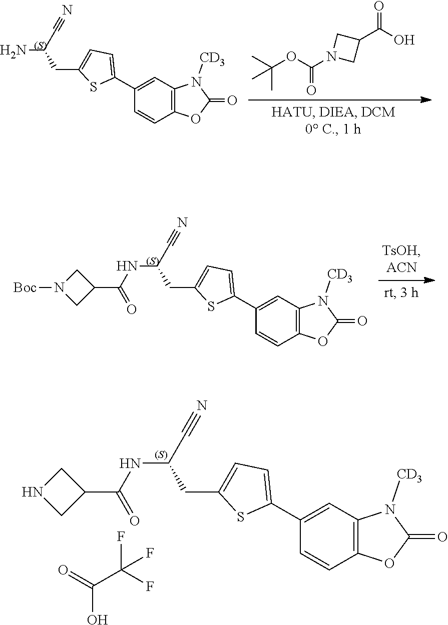

### Synthesis of tert-butyl 3-{[(1S)-1-cyano-2-{5-[3-(2H3)methyl-2-oxo-1,3-benzoxazol-5-yl]thiophen-2-yl}ethyl]carbamoyl}azetidine-1-carboxylate

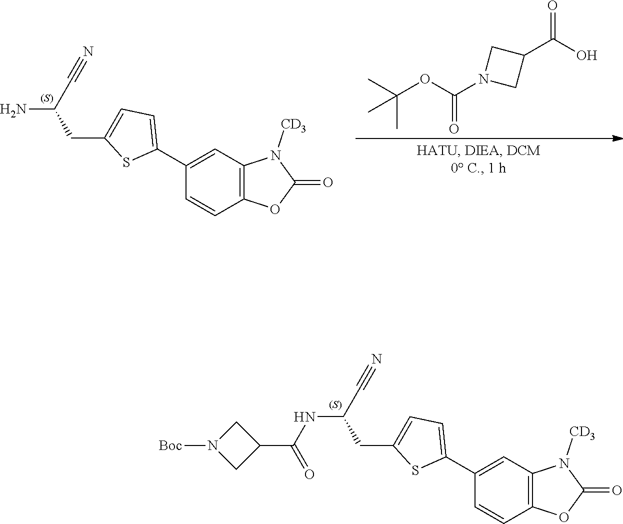

To a stirred solution of (2S)-2-amino-3-{5-[3-(2H3)methyl-2-oxo-1,3-benzoxazol-5-yl]thiophen-2-yl}propanenitrile (108 mg, 0.36 mmol, 1.2 equiv) and 1-(tert-butoxycarbonyl) azetidine-3-carboxylic acid (60 mg, 0.30 mmol, 1.0 equiv) in DCM (2 mL) was added DIEA (115 mg, 0.89 mmol, 3.0 equiv). To the above mixture was added HATU (136 mg, 0.36 mmol, 1.2 equiv) in portions at 0° C. The resulting mixture was stirred for additional 1 h at 0° C. The resulting mixture was concentrated under reduced pressure to remove the solvent. The residue was purified by silica gel column chromatography, eluted with PE/EA (1:1) to afford tert-butyl 3-{[(1S)-1-cyano-2-{5-[3-(2H3)methyl-2-oxo-1,3-benzoxazol-5-yl]thiophen-2-yl}ethyl]carbamoyl}azetidine-1-carboxylate (100 mg, 69%) as off-white solid. LCMS (ES) [M+1]⁺ m/z: 486.

### Synthesis of (S)—N-(1-cyano-2-(5-(3-(methyl-d3)-2-oxo-2,3-dihydrobenzo[d]oxazol-5-yl)thiophen-2-yl)ethyl)azetidine-3-carboxamide 2,2,2-trifluoroacetate

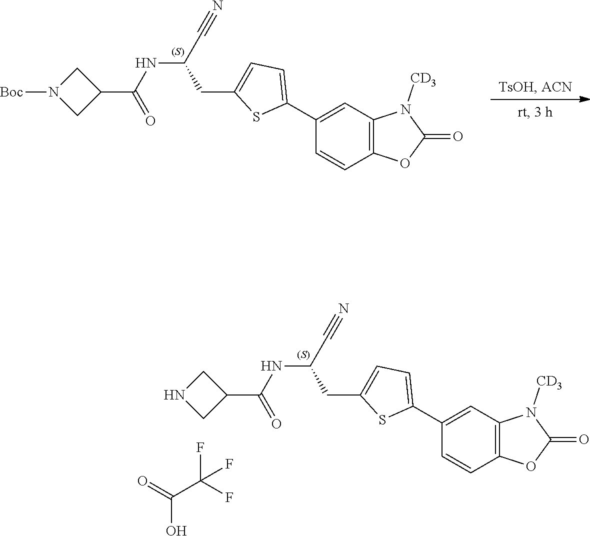

To a stirred solution of tert-butyl 3-{[(1S)-1-cyano-2-{5-[3-(2H3)methyl-2-oxo-1,3-benzoxazol-5-yl]thiophen-2-yl}ethyl]carbamoyl}azetidine-1-carboxylate (90 mg, 0.18 mmol, 1.0 equiv) a in ACN (2 mL) was added TsOH (95 mg, 0.55 mmol, 3.0 equiv). The resulting mixture was stirred for 3 h at room temperature. The reaction solution was purified by Prep-HPLC with the following conditions: Column, Kinetex EVO C18 Column, 21.2*150 mm, 5 um; mobile phase, Water (0.1% TFA) and ACN (10% Phase B up to 50% in 15 min); Detector, UV 254 nm. The fraction of the target was freezing dried to afford(S)—N-(1-cyano-2-(5-(3-(methyl-d3)-2-oxo-2,3-dihydrobenzo[d]oxazol-5-yl)thiophen-2-yl)ethyl)azetidine-3-carboxamide 2,2,2-trifluoroacetate (20 mg, 28%) as a white solid.

¹H NMR (300 MHz, DMSO-d₆) δ 9.17 (d, J=7.6 Hz, 1H), 8.81 (brs, 2H), 7.53 (t, J=1.1 Hz, 1H), 7.41 (d, J=3.6 Hz, 1H), 7.41-7.29 (m, 2H), 7.05 (d, J=3.6 Hz, 1H), 5.04 (q, J=7.4 Hz, 1H), 4.12-4.00 (m, 3H), 3.91 (t, J=8.9 Hz, 1H), 3.66-3.54 (m, 1H), 3.44-3.23 (m, 2H). LCMS (ES) [M−TFA+H]⁺ m/z: 386

### Example 23. Synthesis of Compound 155: (S)—N-(1-cyano-2-(2-fluoro-4-(3-methyl-2-oxo-2,3-dihydrobenzo[d]oxazol-5-yl)phenyl)ethyl)azetidine-3-carboxamide 2,2,2-trifluoroacetate

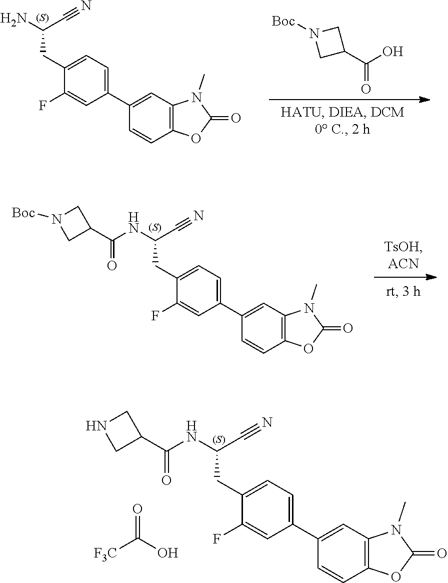

### Synthesis of tert-butyl 3-{[(1S)-1-cyano-2-[2-fluoro-4-(3-methyl-2-oxo-1,3-benzoxazol-5-yl)phenyl]ethyl]carbamoyl}azetidine-1-carboxylate

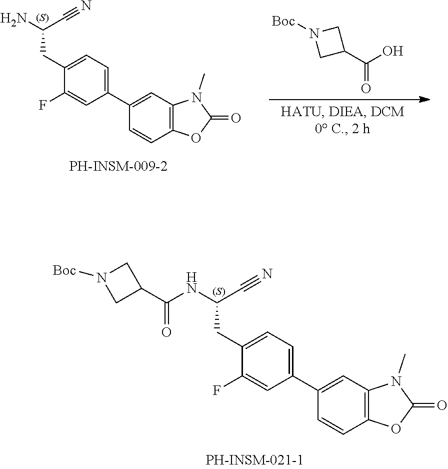

A solution of 1-(tert-butoxycarbonyl) azetidine-3-carboxylic acid (60 mg, 0.30 mmol, 1.0 equiv) in DCM (5 mL) was treated with (2S)-2-amino-3-[2-fluoro-4-(3-methyl-2-oxo-1,3-benzoxazol-5-yl)phenyl]propanenitrile (111 mg, 0.35 mmol, 1.2 equiv), DIEA (116 mg, 0.89 mmol, 3.0 equiv). This was followed by the addition of HATU (136 mg, 0.35 mmol, 1.2 equiv) in portions at 0° C. The resulting mixture was stirred for additional 2 h at 0° C. Concentrated under reduced pressure, the residue was purified by silica gel column chromatography, eluted with PE/THF (1:1) to afford tert-butyl 3-{[(1S)-1-cyano-2-[2-fluoro-4-(3-methyl-2-oxo-1,3-benzoxazol-5-yl)phenyl]ethyl]carbamoyl}azetidine-1-carboxylate (90 mg, 61%) as white solid. LCMS (ES, m/z): [M+H]⁺: 495.

### Synthesis of (S)—N-(1-cyano-2-(2-fluoro-4-(3-methyl-2-oxo-2,3-dihydrobenzo[d]oxazol-5-yl)phenyl)ethyl)azetidine-3-carboxamide 2,2,2-trifluoroacetate

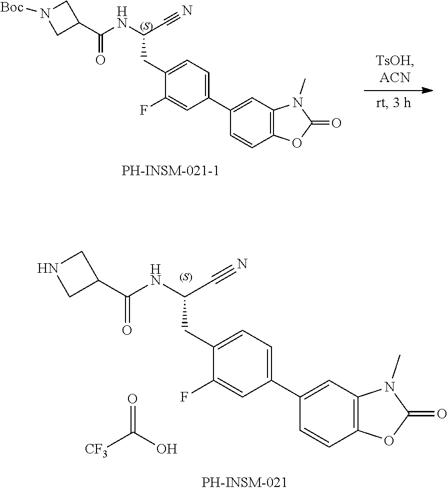

Into a 25 mL round-bottom flask were added tert-butyl 3-{[(1S)-1-cyano-2-[2-fluoro-4-(3-methyl-2-oxo-1,3-benzoxazol-5-yl)phenyl]ethyl]carbamoyl}azetidine-1-carboxylate (90 mg, 0.18 mmol, 1.0 equiv), ACN (3 mL) and TsOH (94 mg, 0.54 mmol, 3.0 equiv) at room temperature. The resulting mixture was stirred for 3 h at room temperature. The reaction solution was purified by reversed-phase flash chromatography with the following conditions: column, C18-120 g, mobile phase, MeCN in Water (0.1% TFA), 10% to 50% gradient in 10 min; detector, UV 254 nm. This resulted in (S)—N-(1-cyano-2-(2-fluoro-4-(3-methyl-2-oxo-2,3-dihydrobenzo[d]oxazol-5-yl)phenyl)ethyl)azetidine-3-carboxamide 2,2,2-trifluoroacetate (22.9 mg, 24.8%) as white solid.

### Analytical Data

LCMS (ES, m/z): [M−TFA+H]⁺: 395.2

¹H NMR (400 MHz, DMSO-d₆) δ 9.16-9.09 (m, 1H), 8.69 (brs, 2H), 7.67 (d, J=1.8 Hz, 1H), 7.65-7.55 (m, 2H), 7.54-7.46 (m, 2H), 7.42 (d, J=8.4 Hz, 1H), 5.09 (q, J=7.7 Hz, 1H), 4.05-3.96 (m, 3H), 3.89-3.84 (m, 1H), 3.60-3.52 (m, 1H), 3.41 (s, 3H), 3.25-3.13 (m, 2H).

### Example 24. Synthesis of Compound 156: (S)—N-(1-cyano-2-(4′-cyano-3-fluoro-[1,1′-biphenyl]-4-yl)ethyl)azetidine-3-carboxamide 2,2,2-trifluoroacetate

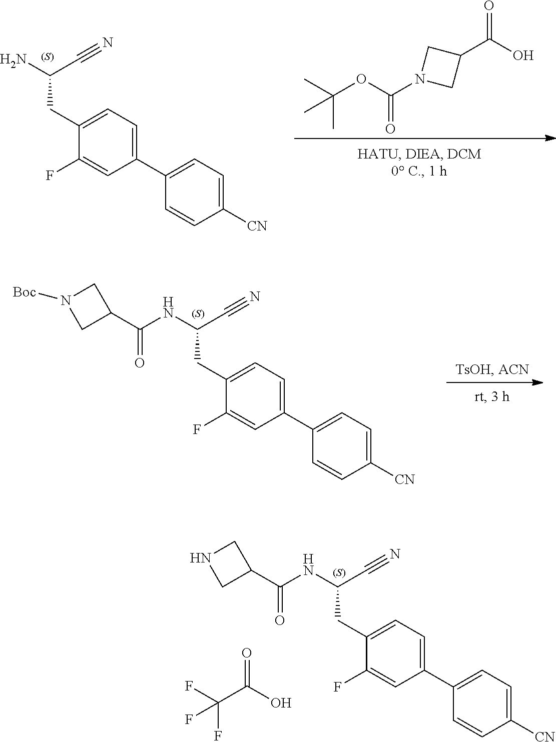

### Synthesis of tert-butyl 3-{[(1S)-1-cyano-2-{4′-cyano-3-fluoro-[1,1′-biphenyl]-4-yl}ethyl]carbamoyl}azetidine-1-carboxylate

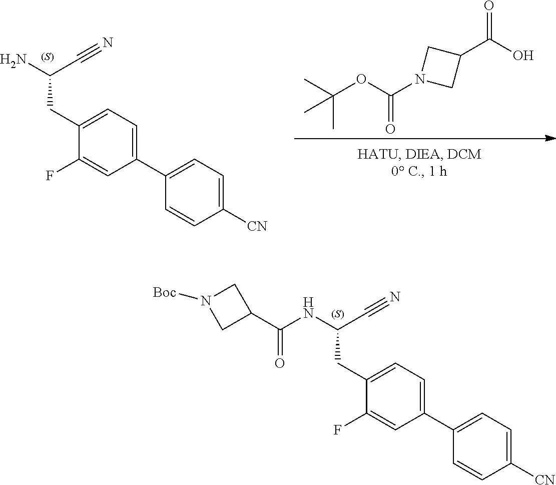

To a stirred solution of 1-(tert-butoxycarbonyl) azetidine-3-carboxylic acid (50 mg, 0.25 mmol, 1.0 equiv) and 4′-[(2S)-2-amino-2-cyanoethyl]-3′-fluoro-[1,1′-biphenyl]-4-carbonitrile (79 mg, 0.30 mmol, 1.2 equiv) in DCM (2 mL) was added DIEA (96 mg, 0.75 mmol, 3.0 equiv). To the above mixture was added HATU (113 mg, 0.30 mmol, 1.2 equiv) in portions at 0° C. The resulting mixture was stirred for additional 1 h at 0° C. Concentrated under reduced pressure to remove the solvent. The residue was purified by silica gel column chromatography, eluted with PE/EA (1:1) to afford tert-butyl 3-{[(1S)-1-cyano-2-{4′-cyano-3-fluoro-[1,1′-biphenyl]-4-yl}ethyl]carbamoyl}azetidine-1-carboxylate (100 mg, 89%) as off-white solid. LCMS (ES) [M+1]⁺ m/z: 449.

### Synthesis of (S)—N-(1-cyano-2-(4′-cyano-3-fluoro-[1,1′-biphenyl]-4-yl)ethyl)azetidine-3-carboxamide 2,2,2-trifluoroacetate

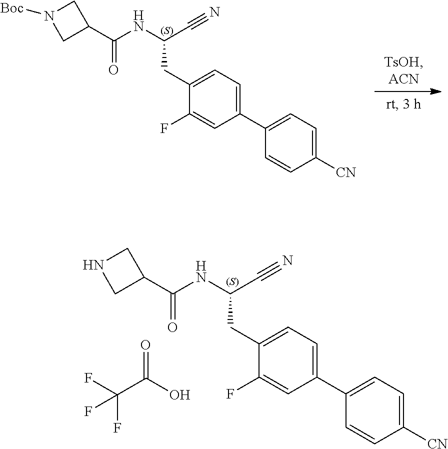

To a stirred solution of tert-butyl 3-{[(1S)-1-cyano-2-{4′-cyano-3-fluoro-[1,1′-biphenyl]-4-yl}ethyl]carbamoyl}azetidine-1-carboxylate (100 mg, 0.23 mmol, 1.0 equiv) in ACN (2 mL) was added TsOH (115 mg, 0.69 mmol, 3.0 equiv). The resulting mixture was stirred for 3 h at room temperature. The reaction solution was purified by Prep-HPLC with the following conditions: Column, Kinetex EVO C18 Column, 21.2*150 mm, 5 um; mobile phase, Water (0.1% TFA) and ACN (10% Phase B up to 50% in 15 min); Detector, UV 254 nm. The fraction of the target was freezing dried to afford(S)—N-(1-cyano-2-(4′-cyano-3-fluoro-[1,1′-biphenyl]-4-yl)ethyl)azetidine-3-carboxamide 2,2,2-trifluoroacetate (25 mg, 25%) as a white solid.

¹H NMR (400 MHz, DMSO-d₆) δ 9.15 (d, J=7.7 Hz, 1H), 8.75 (brs, 2H), 7.95 (s, 4H), 7.69 (dd, J=11.4, 1.8 Hz, 1H), 7.64 (dd, J=8.0, 1.8 Hz, 1H), 7.54 (t, J=7.9 Hz, 1H), 5.10 (q, J=7.6 Hz, 1H), 4.09-3.94 (m, 3H), 3.85 (dd, J=10.5, 7.2 Hz, 1H), 3.63-3.50 (m, 1H), 3.29-3.13 (m, 2H). LCMS (ES) [M+1]⁺ m/z: 349

### Example 25. Synthesis of Compound 157: (S)—N-(1-cyano-2-(2-fluoro-4-(3-(methyl-d3)-2-oxo-2,3-dihydrobenzo[d]oxazol-5-yl)phenyl)ethyl)azetidine-3-carboxamide 2,2,2-trifluoroacetate

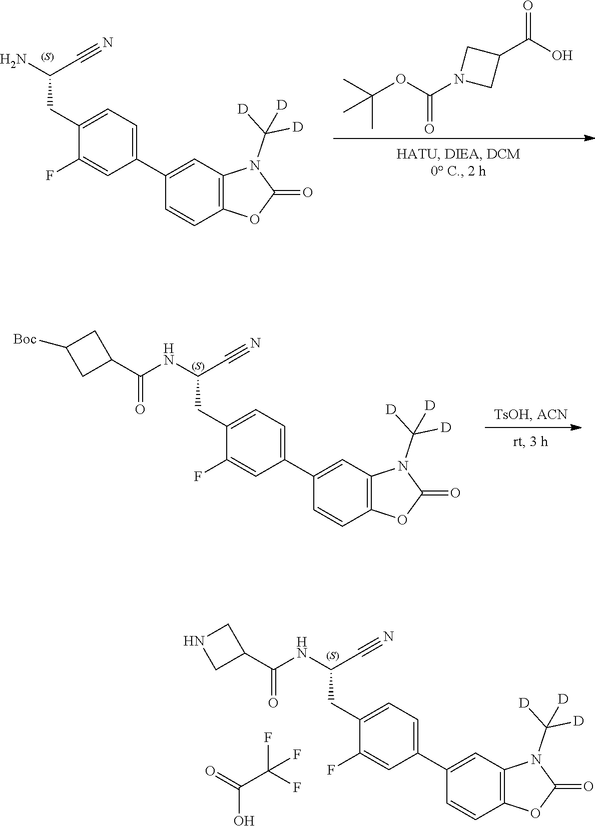

### Synthesis of tert-butyl 3-{[(1S)-1-cyano-2-{2-fluoro-4-[3-(2H3)methyl-2-oxo-1,3-benzoxazol-5-yl]phenyl}ethyl]carbamoyl}azetidine-1-carboxylate

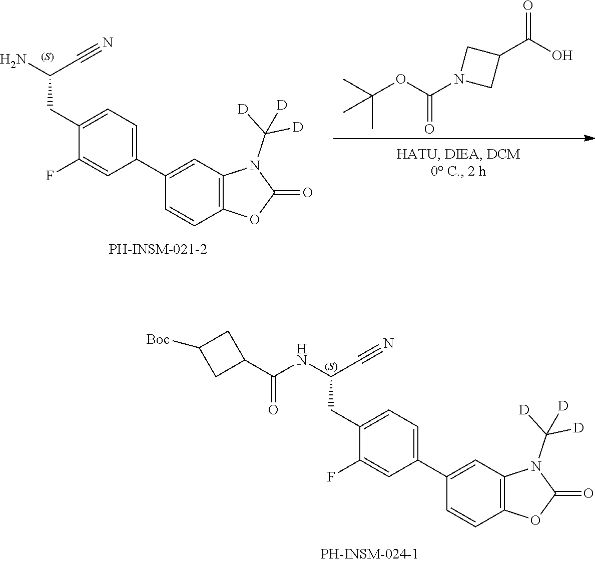

To a stirred mixture of (2S)-2-amino-3-{2-fluoro-4-[3-(2H3)methyl-2-oxo-1,3-benzoxazol-5-yl]phenyl}propanenitrile (112 mg, 0.35 mmol, 1.2 equiv), 1-(tert-butoxycarbonyl) azetidine-3-carboxylic acid (60 mg, 0.298 mmol, 1.00 equiv) and DIEA (116 mg, 0.89 mmol, 3.0 equiv) in DCM (5 mL) was added HATU (136 mg, 0.35 mmol, 1.2 equiv) in portions at 0° C. The resulting mixture was stirred for additional 2 h at 0° C. The residue was purified by silica gel column chromatography, eluted with PE/THF (1:1) to afford tert-butyl 3-{[(1S)-1-cyano-2-{2-fluoro-4-[3-(2H3)methyl-2-oxo-1,3-benzoxazol-5-yl]phenyl}ethyl]carbamoyl}azetidine-1-carboxylate (100 mg, 67.4%) as white solid. LCMS (ES, m/z): [M+H]⁺: 497.

### Synthesis of (S)—N-(1-cyano-2-(2-fluoro-4-(3-(methyl-d3)-2-oxo-2,3-dihydrobenzo[d]oxazol-5-yl)phenyl)ethyl)azetidine-3-carboxamide 2,2,2-trifluoroacetate

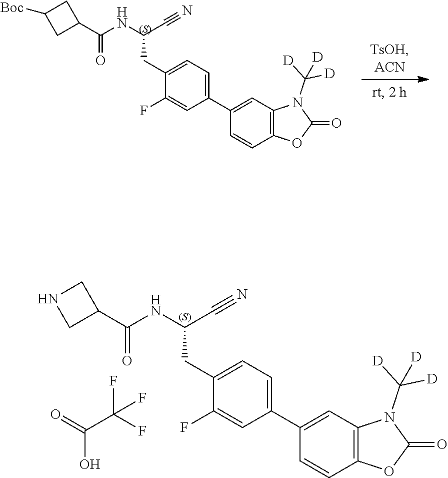

Into a 25 mL round-bottom flask were added tert-butyl 3-{[(1S)-1-cyano-2-{2-fluoro-4-[3-(2H3)methyl-2-oxo-1,3-benzoxazol-5-yl]phenyl}ethyl]carbamoyl}azetidine-1-carboxylate (100 mg, 0.20 mmol, 1.0 equiv) in ACN (3 mL) and TsOH (104 mg, 0.60 mmol, 3.0 equiv) at room temperature. The resulting mixture was stirred for 3 h at room temperature. The reaction solution was purified by reversed-phase flash chromatography with the following conditions: column, C18-120 g, mobile phase, MeCN in Water (0.1% TFA), 10% to 80% gradient in 10 min, detector, UV 254 nm. This resulted in (S)—N-(1-cyano-2-(2-fluoro-4-(3-(methyl-d3)-2-oxo-2,3-dihydrobenzo[d]oxazol-5-yl)phenyl)ethyl)azetidine-3-carboxamide 2,2,2-trifluoroacetate (20.0 mg, 20%) as white solid.

LCMS (ES, m/z): [M−TFA+H]⁺: 398.3

¹H NMR (400 MHz, DMSO-d₆) δ 9.15 (d, J=7.7 Hz, 1H), 8.74 (brs, 2H), 7.67 (d, J=1.9 Hz, 1H), 7.65-7.55 (m, 2H), 7.54-7.46 (m, 2H), 7.42 (d, J=8.3 Hz, 1H), 5.09 (q, J=7.6 Hz, 1H), 4.09-3.95 (m, 3H), 3.89-3.84 (m, 1H), 3.61-3.52 (m, 1H), 3.25-3.13 (m, 2H).

### Example 26. Synthesis of Compound 187: (2S,3aS,6aR)—N-[(1S)-1-cyano-2-[4-(3-methyl-2-oxo-1,3-benzoxazol-5-yl)phenyl]ethyl]-hexahydro-1H-furo[3,4-b]pyrrole-2-carboxamide

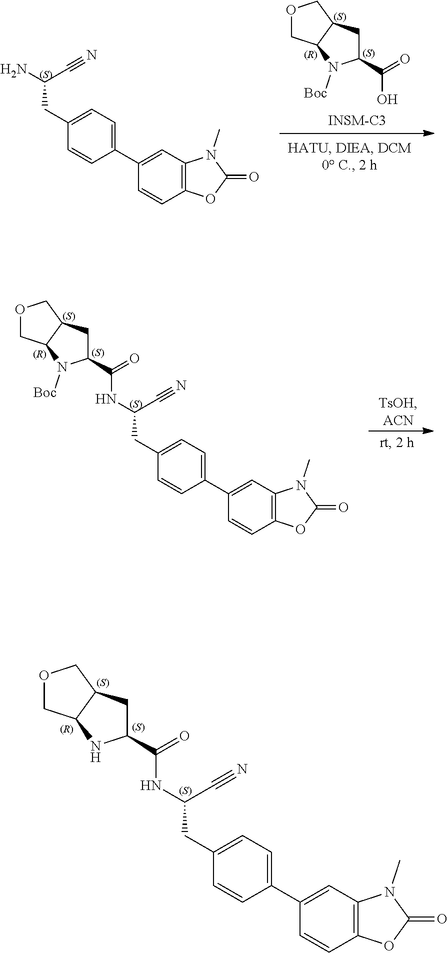

### Synthesis of tert-butyl (2S,3aS,6aR)-2-{[(1S)-1-cyano-2-[4-(3-methyl-2-oxo-1,3-benzoxazol-5-yl)phenyl]ethyl]carbamoyl}-hexahydrofuro[3,4-b]pyrrole-1-carboxylate

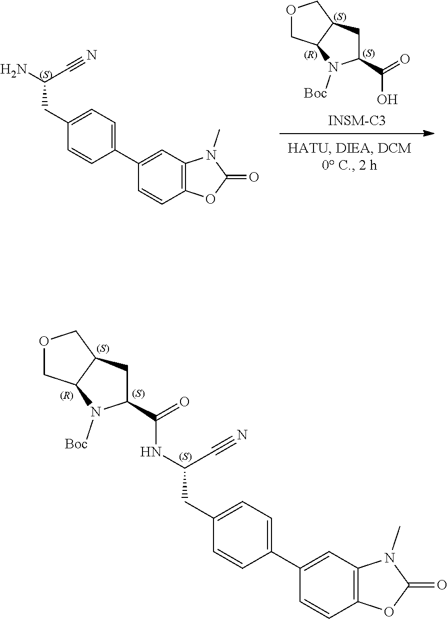

A solution of (2S,3aS,6aR)-1-(tert-butoxycarbonyl)-hexahydrofuro[3,4-b]pyrrole-2-carboxylic acid (60 mg, 0.23 mmol, 1.0 equiv) in DCM (5 mL) was treated with (2S)-2-amino-3-[4-(3-methyl-2-oxo-1,3-benzoxazol-5-yl)phenyl]propanenitrile (82 mg, 0.28 mmol, 1.2 equiv), DIEA (90 mg, 0.69 mmol, 3.0 equiv) followed by the addition of HATU (106 mg, 0.28 mmol, 1.2 equiv) in portions at 0° C. The resulting mixture was stirred for additional 2 h at 0° C. Concentrated under reduced pressure to remove the solvent, the residue was purified by silica gel column chromatography, eluted with PE/THF (1:1) to afford tert-butyl (2S,3aS,6aR)-2-{[(1S)-1-cyano-2-[4-(3-methyl-2-oxo-1,3-benzoxazol-5-yl)phenyl]ethyl]carbamoyl}-hexahydrofuro[3,4-b]pyrrole-1-carboxylate (100 mg, 80.5%) as white solid. LCMS (ES, m/z): [M+H]⁺: 533.

### Synthesis of (2S,3aS,6aR)—N-[(1S)-1-cyano-2-[4-(3-methyl-2-oxo-1,3-benzoxazol-5-yl)phenyl]ethyl]-hexahydro-1H-furo[3,4-b]pyrrole-2-carboxamide

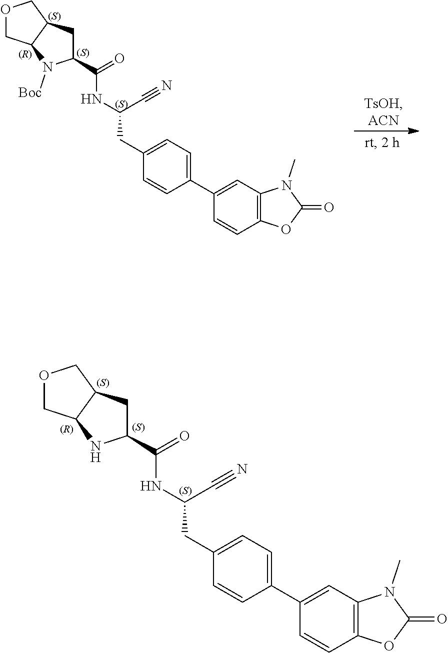

Into a 50 mL round-bottom flask were added tert-butyl (2S,3aS,6aR)-2-{[(1S)-1-cyano-2-[4-(3-methyl-2-oxo-1,3-benzoxazol-5-yl)phenyl]ethyl]carbamoyl}-hexahydrofuro[3,4-b]pyrrole-1-carboxylate (100 mg, 0.18 mmol, 1.0 equiv), TsOH (97 mg, 0.56 mmol, 3.0 equiv) and ACN (3 mL) at room temperature. The resulting mixture was stirred for additional 2 h at room temperature. The residue was purified by reversed-phase flash chromatography with the following conditions: column, C18-120 g, mobile phase, MeCN in Water (0.1% NH₃·H₂O), 10% to 80% gradient in 10 min; detector, UV 254 nm. This resulted in (2S,3aS,6aR)—N-[(1S)-1-cyano-2-[4-(3-methyl-2-oxo-1,3-benzoxazol-5-yl)phenyl]ethyl]-hexahydro-1H-furo[3,4-b]pyrrole-2-carboxamide (27.1 mg, 33.4%) as white solid.

### Analytical Data

LCMS (ES, m/z): [M+H]⁺: 433.2

¹H NMR (400 MHz, DMSO-d₆) δ 8.53 (d, J=8.4 Hz, 1H), 7.66 (d, J=7.9 Hz, 2H), 7.58 (s, 1H), 7.43-7.39 (m, 4H), 5.02 (q, J=7.8 Hz, 1H), 3.85-3.79 (m, 1H), 3.67-3.56 (m, 2H), 3.49-3.32 (m, 6H), 3.25-3.19 (m, 3H), 2.73-2.66 (m, 1H), 2.16-2.04 (m, 1H), 1.47-1.41 (m, 1H).

### Example 27. Synthesis of Compound 183: (2S,3aS,6aR)—N-[(1S)-1-cyano-2-{4′-cyano-[1,1′-biphenyl]-4-yl}ethyl]-hexahydro-1H-furo[3,4-b]pyrrole-2-carboxamide

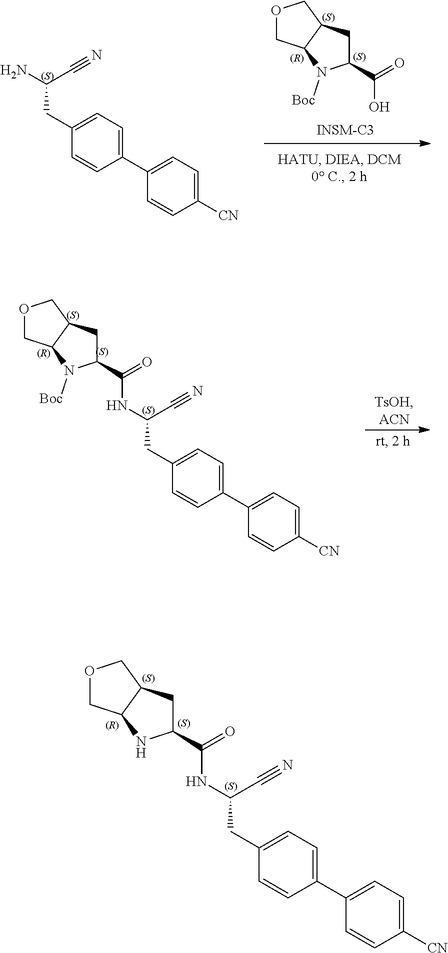

### Synthesis of tert-butyl (2S,3aS,6aR)-2-{[(1S)-1-cyano-2-{4′-cyano-[1,1′-biphenyl]-4-yl}ethyl]carbamoyl}-hexahydrofuro[3,4-b]pyrrole-1-carboxylate

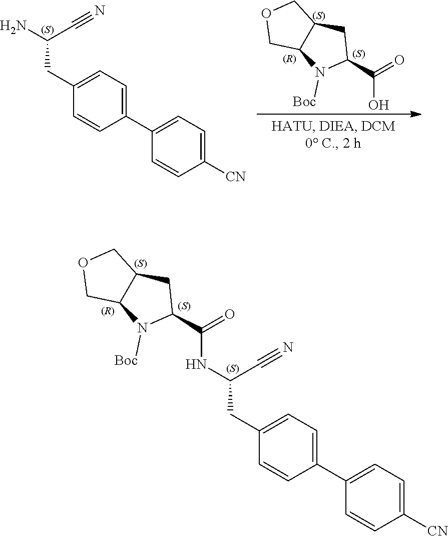

To a stirred mixture of (2S,3aS,6aR)-1-(tert-butoxycarbonyl)-hexahydrofuro[3,4-b]pyrrole-2-carboxylic acid (70 mg, 0.27 mmol, 1.0 equiv) and 4′-[(2S)-2-amino-2-cyanoethyl]-[1,1′-biphenyl]-4-carbonitrile (81 mg, 0.33 mmol, 1.2 equiv), DIEA (105 mg, 0.82 mmol, 3.0 equiv) in DCM (3 mL) was added HATU (124 mg, 0.33 mmol, 1.2 equiv) at 0° C. The resulting mixture was stirred for 2 h at 0° C. The residue was purified by silica gel column chromatography, eluted with PE/THF (2:1) to afford tert-butyl (2S,3aS,6aR)-2-{[(1S)-1-cyano-2-{4′-cyano-[1,1′-biphenyl]-4-yl}ethyl]carbamoyl}-hexahydrofuro[3,4-b]pyrrole-1-carboxylate (100 mg, 75%) as a light yellow oil. LCMS (ES, m/z): [M+H]⁺: 487.

### Synthesis of (2S,3aS,6aR)—N-[(1S)-1-cyano-2-{4′-cyano-[1,1′-biphenyl]-4-yl}ethyl]-hexahydro-1H-furo[3,4-b]pyrrole-2-carboxamide

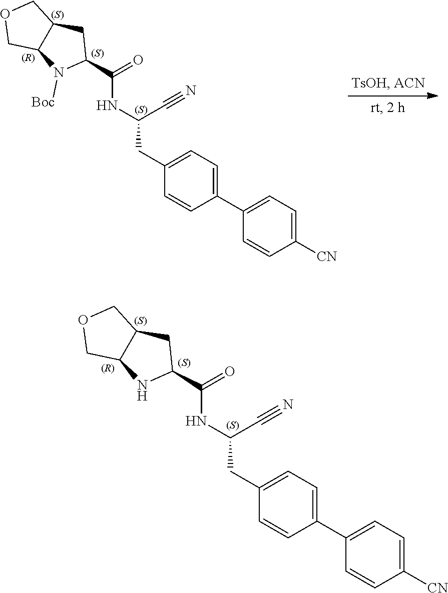

Into a 50 mL round-bottom flask were added tert-butyl (2S,3aS,6aR)-2-{[(1S)-1-cyano-2-{4′-cyano-[1, l′-biphenyl]-4-yl}ethyl]carbamoyl}-hexahydrofuro[3,4-b]pyrrole-1-carboxylate (100 mg, 0.21 mmol, 1.0 equiv), ACN (3 mL) and TsOH·H₂O (117 mg, 0.62 mmol, 3.0 equiv) at room temperature. The resulting mixture was stirred for 2 h at room temperature. The reaction solution was purified by Prep-HPLC with the following conditions: Column, Atlantis Prep T3 OBD Column, 19*150 mm, 5 um; mobile phase, Water (0.05% NH₃·H₂O) and ACN (25% Phase B up to 50% in 10 min) to afford (2S,3aS,6aR)—N-[(1S)-1-cyano-2-{4′-cyano-[1,1′-biphenyl]-4-yl}ethyl]-hexahydro-1H-furo[3,4-b]pyrrole-2-carboxamide (21.7 mg, 27%) as a white solid.

### Analytical Data

LCMS (ES, m/z): [M+H]⁺: 387.

¹H NMR (400 MHz, DMSO-d₆) δ 8.53 (d, J=8.4 Hz, 1H), 7.93 (d, J=8.1 Hz, 2H), 7.88 (d, J=8.2 Hz, 2H), 7.72 (d, J=7.9 Hz, 2H), 7.44 (d, J=7.9 Hz, 2H), 5.03 (q, J=7.9 Hz, 1H), 3.85-3.79 (m, 1H), 3.63-3.56 (m, 2H), 3.48-3.42 (m, 2H), 3.35-3.33 (m, 1H), 3.24-3.19 (m, 3H), 2.70-2.64 (m, 1H), 2.12-2.05 (m, 1H), 1.44-1.37 (m, 1H).

### Example 28. Synthesis of Compound 300: (2S,3aS,6aR)—N—((S)-1-cyano-2-(4-(3-(methyl-d3)-2-oxo-2,3-dihydrobenzo[d]oxazol-5-yl)phenyl)ethyl)hexahydro-1H-furo[3,4-b]pyrrole-2-carboxamide

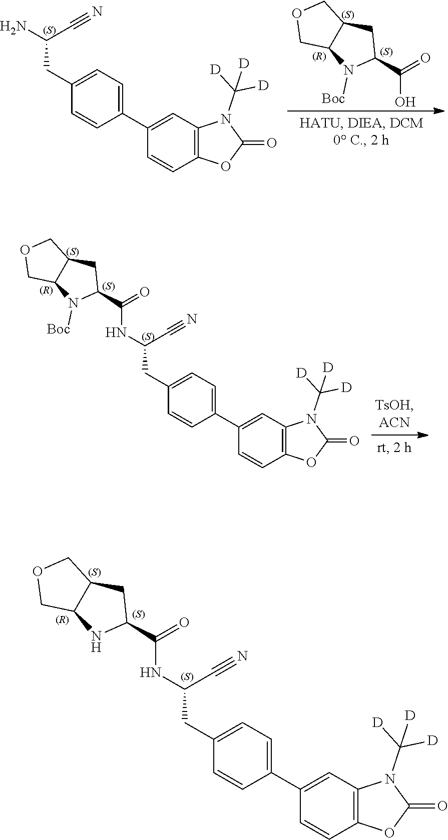

### Synthesis of tert-butyl (2S,3aS,6aR)-2-{[(1S)-1-cyano-2-{4-[3-(2H3)methyl-2-oxo-1,3-benzoxazol-5-yl]phenyl}ethyl]carbamoyl}-hexahydrofuro[3,4-b]pyrrole-1-carboxylate

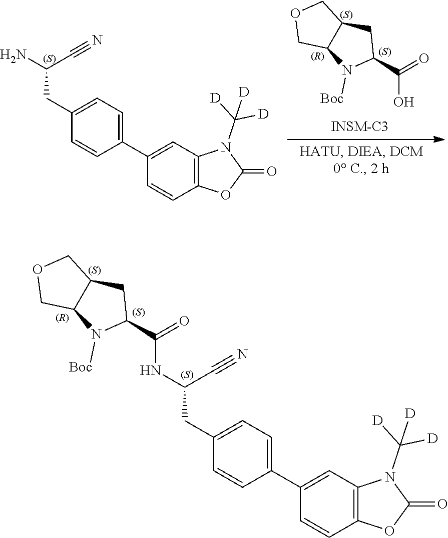

A solution of (2S)-2-amino-3-{4-[3-(2H3)methyl-2-oxo-1,3-benzoxazol-5-yl]phenyl}propanenitrile (88.69 mg, 0.30 mmol, 1.1 equiv) in DCM (5 mL) was treated with (2S,3aS,6aR)-1-(tert-butoxycarbonyl)-hexahydrofuro[3,4-b]pyrrole-2-carboxylic acid (70 mg, 0.27 mmol, 1.0 equiv), DIEA (105 mg, 0.81 mmol, 3.0 equiv) at room temperature. This was followed by the addition of HATU (124 mg, 0.32 mmol, 1.2 equiv) in portions at 0° C. The resulting mixture was stirred for 2 h at 0° C. Concentrated under reduced pressure, the residue was purified by silica gel column chromatography, eluted with PE/THF (1:1) to afford tert-butyl (2S,3aS,6aR)-2-{[(1S)-1-cyano-2-{4-[3-(2H3)methyl-2-oxo-1,3-benzoxazol-5-yl]phenyl}ethyl]carbamoyl}-hexahydrofuro[3,4-b]pyrrole-1-carboxylate (85 mg, 58%) as white solid. LCMS (ES, m/z): [M+H]⁺: 536

### Synthesis of (2S,3aS,6aR)—N-[(1S)-1-cyano-2-{4-[3-(2H3)methyl-2-oxo-1,3-benzoxazol-5-yl]phenyl}ethyl]-hexahydro-1H-furo[3,4-b]pyrrole-2-carboxamide

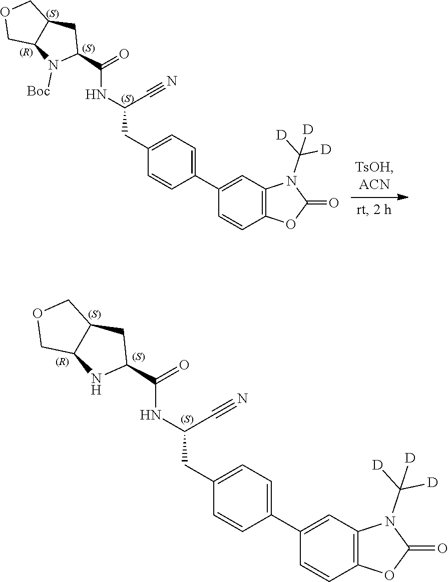

Into a 25 mL round-bottom flask were added tert-butyl (2S,3aS,6aR)-2-{[(1S)-1-cyano-2-{4-[3-(2H3)methyl-2-oxo-1,3-benzoxazol-5-yl]phenyl}ethyl]carbamoyl}-hexahydrofuro[3,4-b]pyrrole-1-carboxylate (85 mg, 0.15 mmol, 1.0 equiv), ACN (3 mL) and TsOH (82 mg, 0.47 mmol, 3.0 equiv) in sequence at room temperature. The resulting mixture was stirred for additional 2 h at room temperature. The reaction was purified by reversed-phase flash chromatography with the following conditions: column, C18-120 g, mobile phase, MeCN in Water (0.1% NH₃·H₂O), 10% to 50% gradient in 10 min; detector, UV 254 nm. This resulted in (2S,3aS,6aR)—N-[(1S)-1-cyano-2-{4-[3-(2H3)methyl-2-oxo-1,3-benzoxazol-5-yl]phenyl}ethyl]-hexahydro-1H-furo[3,4-b]pyrrole-2-carboxamide (13.9 mg, 20%) as white solid.

### Analytical Data

LCMS (ES, m/z): [M+H]⁺: 436.5

¹H NMR (400 MHz, DMSO-d₆) δ 8.53 (d, J=8.4 Hz, 1H), 7.69-7.62 (m, 2H), 7.58 (t, J=1.1 Hz, 1H), 7.46-7.36 (m, 4H), 5.02 (q, J=7.8 Hz, 1H), 3.86-3.78 (m, 1H), 3.64 (dd, J=9.0, 2.0 Hz, 1H), 3.59 (t, J=7.6 Hz, 1H), 3.52-3.34 (m, 2H), 3.38 (dd, J=8.8, 3.5 Hz, 1H), 3.19 (d, J=7.7 Hz, 2H), 2.76-2.67 (m, 1H), 2.14-2.06 (m, 1H), 1.47-1.41 (m, 1H).

### Example 29. Synthesis of Compound 192: (2S,3aS,6aR)—N-[(1S)-1-cyano-2-[5-(3-methyl-2-oxo-1,3-benzoxazol-5-yl)thiophen-2-ylethyl]-hexahydro-1H-furo[3,4-b]pyrrole-2-carboxamide

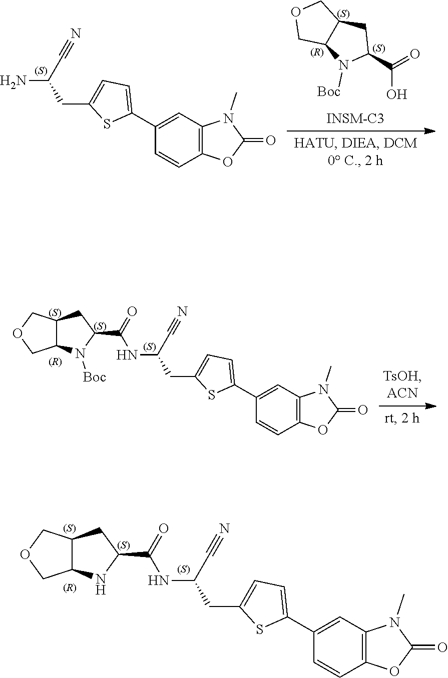

### Synthesis of tert-butyl (2S,3aS,6aR)-2-{[(1S)-1-cyano-2-[5-(3-methyl-2-oxo-1,3-benzoxazol-5-yl)thiophen-2-yl]ethyl]carbamoyl}-hexahydrofuro[3,4-b]pyrrole-1-carboxylate

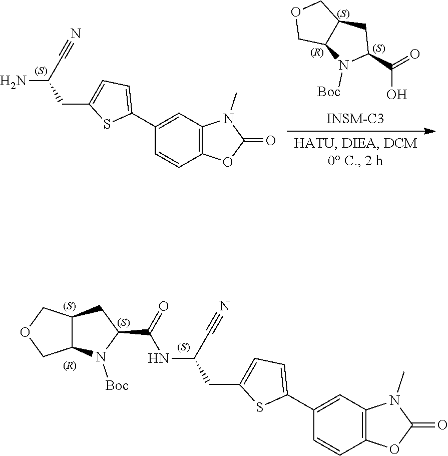

A solution of (2S,3aS,6aR)-1-(tert-butoxycarbonyl)-hexahydrofuro[3,4-b]pyrrole-2-carboxylic acid (60 mg, 0.23 mmol, 1.0 equiv) in DCM (5 mL) was treated with (2S)-2-amino-3-[5-(3-methyl-2-oxo-1,3-benzoxazol-5-yl)thiophen-2-yl]propanenitrile (84 mg, 0.28 mmol, 1.2 equiv), DIEA (90 mg, 0.69 mmol, 3.0 equiv). This was followed by the addition of HATU (106 mg, 0.28 mmol, 1.2 equiv) in portions at 0° C. The resulting mixture was stirred for additional 2 h at 0° C. Concentrated under reduced pressure to remove the solvent, the residue was purified by silica gel column chromatography, eluted with PE/THF (1:1) to afford tert-butyl (2S,3aS,6aR)-2-{[(1S)-1-cyano-2-[5-(3-methyl-2-oxo-1,3-benzoxazol-5-yl)thiophen-2-yl]ethyl]carbamoyl}-hexahydrofuro[3,4-b]pyrrole-1-carboxylate (90 mg, 71.6%) as white solid. LCMS (ES, m/z): [M+H]⁺: 539.

### Synthesis of (2S)—N-[(1S)-1-cyano-2-{4′-[(4-methylpiperazin-1-yl)methyl]-[1,1′-biphenyl]-4-yl}ethyl]-1,4-oxazepane-2-carboxamide

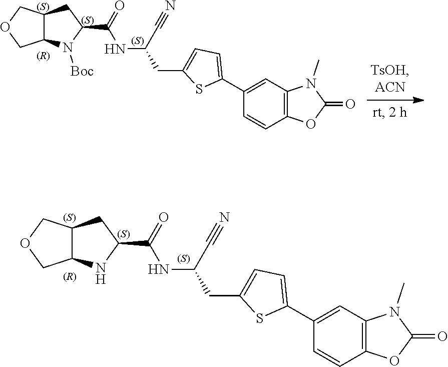

Into a 25 mL round-bottom flask were added tert-butyl (2S,3aS,6aR)-2-{[(1S)-1-cyano-2-[5-(3-methyl-2-oxo-1,3-benzoxazol-5-yl)thiophen-2-yl]ethyl]carbamoyl}-hexahydrofuro[3,4-b]pyrrole-1-carboxylate (90 mg, 0.16 mmol, 1.0 equiv), TsOH (86 mg, 0.50 mmol, 3.0 equiv) and ACN (3 mL) at room temperature. The resulting mixture was stirred for additional 2 h at room temperature. The reaction solution was purified by reversed-phase flash chromatography with the following conditions: column, C18-120 g, mobile phase, MeCN in Water (0.1% NH₃·H₂O), 10% to 50% gradient in 10 min, detector, UV 254 nm. This resulted in (2S,3aS,6aR)—N-[(1S)-1-cyano-2-[5-(3-methyl-2-oxo-1,3-benzoxazol-5-yl)thiophen-2-yl]ethyl]-hexahydro-1H-furo[3,4-b]pyrrole-2-carboxamide (19.5 mg, 26.6%) as white solid.

### Analytical Data

LCMS (ES, m/z): [M+H]⁺: 439.2

¹H NMR (400 MHz, DMSO-d₆) δ 8.62 (d, J=8.5 Hz, 1H), 7.53 (s, 1H), 7.40 (d, J=3.7 Hz, 1H), 7.36-7.33 (m, 2H), 7.02 (d, J=3.6 Hz, 1H), 5.01 (q, J=7.7 Hz, 1H), 3.85 (t, J=6.2 Hz, 1H), 3.70 (d, J=9.0 Hz, 1H), 3.64 (t, J=7.7 Hz, 1H), 3.51-3.36 (m, 8H), 2.75-2.66 (m, 1H), 2.17-2.10 (m, 1H), 1.58-1.52 (m, 1H).

### Example 30. Synthesis of Compound 193: (2S,3aS,6aR)—N-[(1S)-1-cyano-2-[5-(4-cyanophenyl)thiophen-2-ylethyl]-hexahydro-1H-furo[3,4-b]pyrrole-2-carboxamide

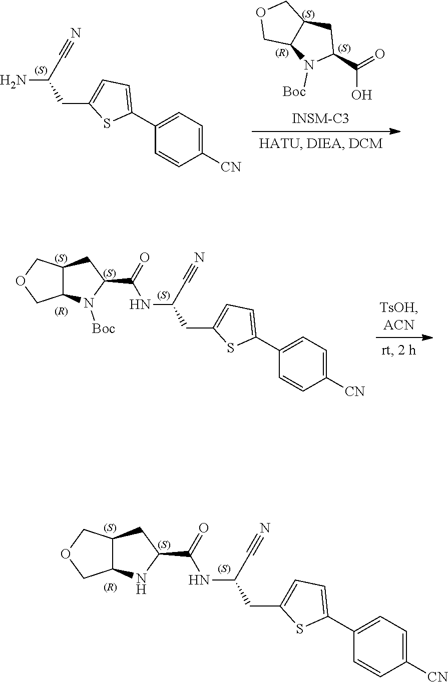

### Synthesis of tert-butyl (2S,3aS,6aR)-2-{[(1S)-1-cyano-2-[5-(4-cyanophenyl)thiophen-2-yl]ethyl]carbamoyl}-hexahydrofuro[3,4-b]pyrrole-1-carboxylate

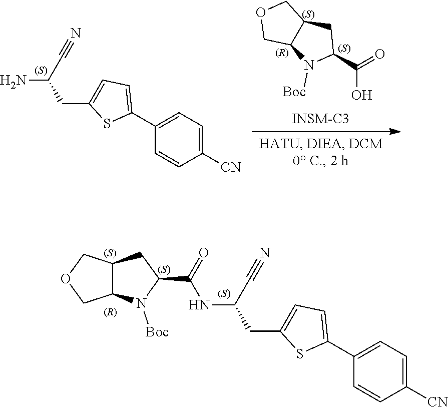

A solution of (2S,3aS,6aR)-1-(tert-butoxycarbonyl)-hexahydrofuro[3,4-b]pyrrole-2-carboxylic acid (60 mg, 0.23 mmol, 1.0 equiv) in DCM (5 mL) was treated with 4-{5-[(2S)-2-amino-2-cyanoethyl]thiophen-2-yl}benzonitrile (71 mg, 0.28 mmol, 1.2 equiv), DIEA (90 mg, 0.69 mmol, 3.0 equiv) followed by the addition of HATU (106 mg, 0.28 mmol, 1.2 equiv) in portions at 0° C. The resulting mixture was stirred for additional 2 h at 0° C. Concentrated to remove the solvent, the residue was purified by silica gel column chromatography, eluted with PE/THF (1:1) to afford tert-butyl (2S,3aS,6aR)-2-{[(1S)-1-cyano-2-[5-(4-cyanophenyl)thiophen-2-yl]ethyl]carbamoyl}-hexahydrofuro[3,4-b]pyrrole-1-carboxylate (90 mg, 78.3%) as white solid. LCMS (ES, m/z): [M+H]⁺: 493.

### Synthesis of (2S,3aS,6aR)—N-[(1S)-1-cyano-2-[5-(4-cyanophenyl)thiophen-2-yl]ethyl]-hexahydro-1H-furo[3,4-b]pyrrole-2-carboxamide

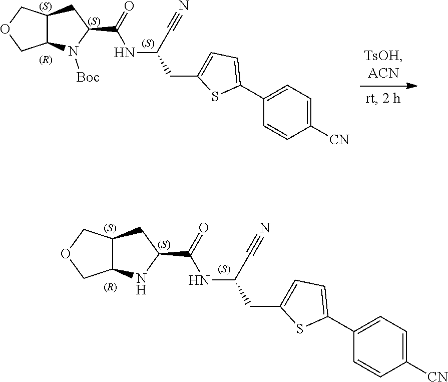

Into a 25 mL round-bottom flask were added tert-butyl (2S,3aS,6aR)-2-{[(1S)-1-cyano-2-[5-(4-cyanophenyl)thiophen-2-yl]ethyl]carbamoyl}-hexahydrofuro[3,4-b]pyrrole-1-carboxylate (90 mg, 0.18 mmol, 1.0 equiv), TsOH (94 mg, 0.54 mmol, 3.0 equiv) and ACN (3 ml) at room temperature. The resulting mixture was stirred for additional 2 h at room temperature. The reaction solution was purified by reversed-phase flash chromatography with the following conditions: column, C18-120 g, mobile phase, MeCN in Water (0.1% NH₃·H₂O), 10% to 50% gradient in 10 min; detector, UV 254 nm. This resulted in (2S,3aS,6aR)—N-[(1S)-1-cyano-2-[5-(4-cyanophenyl)thiophen-2-yl]ethyl]-hexahydro-1H-furo[3,4-b]pyrrole-2-carboxamide (14.7 mg, 20.5%) as white solid.

### Analytical Data

LCMS (ES, m/z): [M+H]⁺: 393.2

¹H NMR (400 MHz, DMSO-d₆) δ 8.62 (d, J=8.5 Hz, 1H), 7.87-7.80 (m, 4H), 7.61 (d, J=3.7 Hz, 1H), 7.09 (d, J=3.7 Hz, 1H), 5.03 (q, J=7.7 Hz, 1H), 3.84 (t, J=7.2 Hz, 1H), 3.71-3.55 (m, 2H), 3.51-3.34 (m, 5H), 3.31-3.24 (m, 1H), 2.75-2.66 (m, 1H), 2.18-2.06 (m, 1H), 1.55-1.48 (m, 1H).

### Example 31. Synthesis of Compound 301: (2S,3aS,6aR)—N—((S)-1-cyano-2-(5-(3-(methyl-d3)-2-oxo-2,3-dihydrobenzo[d]oxazol-5-yl)thiophen-2-yl)ethyl)hexahydro-1H-furo[3,4-b]pyrrole-2-carboxamide

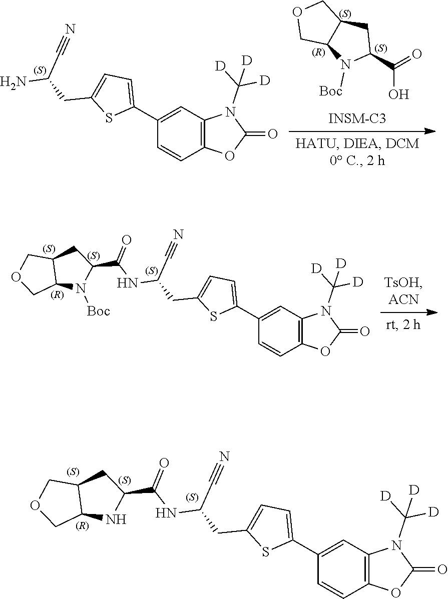

### Synthesis of tert-butyl (2S,3aS,6aR)-2-{[(1S)-1-cyano-2-{5-[3-(2H3)methyl-2-oxo-1,3-benzoxazol-5-yl]thiophen-2-yl}ethyl]carbamoyl}-hexahydrofuro[3,4-b]pyrrole-1-carboxylate

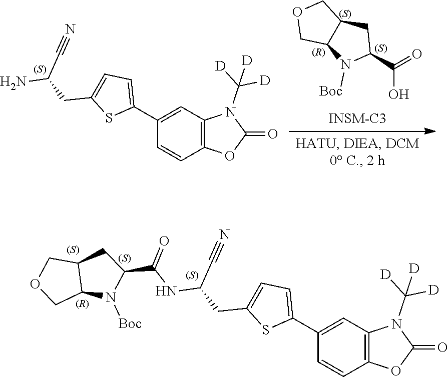

A solution of (2S)-2-amino-3-{5-[3-(2H3)methyl-2-oxo-1,3-benzoxazol-5-yl]thiophen-2-yl}propanenitrile (90 mg, 0.29 mmol, 1.1 equiv) in DCM (5 mL) was treated with (2S,3aS,6aR)-1-(tert-butoxycarbonyl)-hexahydrofuro[3,4-b]pyrrole-2-carboxylic acid (70 mg, 0.27 mmol, 1.0 equiv), DIEA (105 mg, 0.81 mmol, 3.0 equiv). This was followed by the addition of HATU (124 mg, 0.32 mmol, 1.2 equiv) in portions at 0° C. The resulting mixture was stirred for 2 h at 0° C. Concentrated under reduced pressure to remove the solvent. The residue was purified by silica gel column chromatography, eluted with PE/THF (1:1) to afford tert-butyl (2S,3aS,6aR)-2-{[(1S)-1-cyano-2-{5-[3-(2H3)methyl-2-oxo-1,3-benzoxazol-5-yl]thiophen-2-yl}ethyl]carbamoyl}-hexahydrofuro[3,4-b]pyrrole-1-carboxylate (80 mg, 54%) as white solid. LCMS (ES, m/z): [M+H]⁺: 542.

### Synthesis of (2S,3aS,6aR)—N—((S)-1-cyano-2-(5-(3-(methyl-d3)-2-oxo-2,3-dihydrobenzo[d]oxazol-5-yl)thiophen-2-yl)ethyl)hexahydro-1H-furo[3,4-b]pyrrole-2-carboxamide

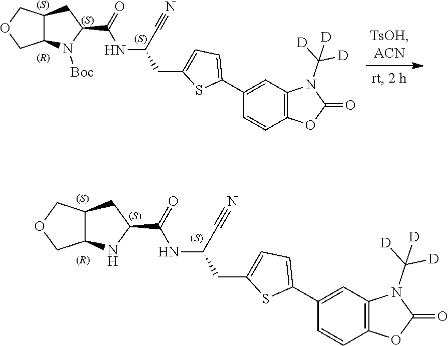

Into a 25 mL round-bottom flask were added tert-butyl (2S,3aS,6aR)-2-{[(1S)-1-cyano-2-{5-[3-(2H3)methyl-2-oxo-1,3-benzoxazol-5-yl]thiophen-2-yl}ethyl]carbamoyl}-hexahydrofuro[3,4-b]pyrrole-1-carboxylate (80 mg, 0.15 mmol, 1.0 equiv) in ACN (3 mL) and TsOH (76 mg, 0.44 mmol, 3.0 equiv) at room temperature. The resulting mixture was stirred for 3 h at room temperature. The reaction solution was purified by reversed-phase flash chromatography with the following conditions: column, C18 silica gel-120 g; mobile phase, MeCN in Water (0.1% NH₃·H₂O), 10% to 80% gradient in 10 min; detector, UV 254 nm. This resulted in (2S,3aS,6aR)—N—((S)-1-cyano-2-(5-(3-(methyl-d3)-2-oxo-2,3-dihydrobenzo[d]oxazol-5-yl)thiophen-2-yl)ethyl)hexahydro-1H-furo[3,4-b]pyrrole-2-carboxamide (16.0 mg, 25%) as white solid.

### Analytical Data

LCMS (ES, m/z): [M+H]⁺: 442.2

¹H NMR (400 MHz, DMSO-d₆) δ 8.61 (d, J=8.5 Hz, 1H), 7.53 (d, J=1.6 Hz, 1H), 7.39 (d, J=3.6 Hz, 1H), 7.38-7.33 (d, J=2.3 Hz, 2H), 7.02 (d, J=3.6 Hz, 1H), 5.01 (q, J=7.7 Hz, 1H), 3.87-3.82 (m, 1H), 3.70 (dd, J=9.0, 2.0 Hz, 1H), 3.67-3.61 (m, 1H), 3.53-3.32 (m, 5H), 2.76-2.70 (m, 1H), 2.20-2.06 (m, 1H), 1.58-1.52 (m, 1H).

### Example 32. Synthesis of Compound 195: (2S,3aS,6aR)—N—((S)-1-cyano-2-(2-fluoro-4-(3-methyl-2-oxo-2,3-dihydrobenzo[d]oxazol-5-yl)phenyl)ethyl)hexahydro-1H-furo[3,4-b]pyrrole-2-carboxamide

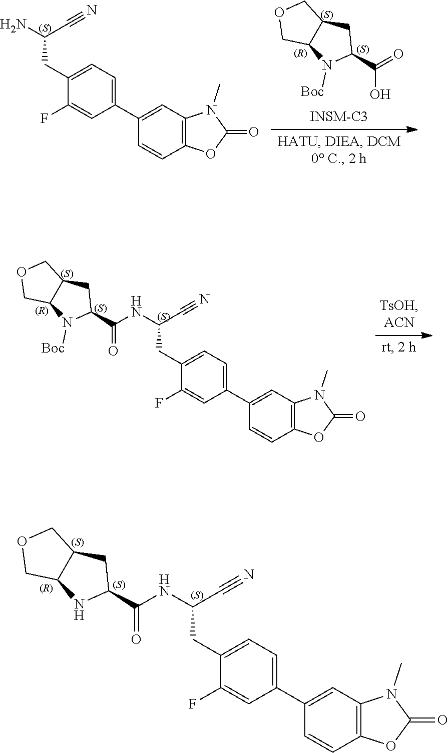

### Synthesis of tert-butyl (2S,3aS,6aR)-2-{[(1S)-1-cyano-2-[2-fluoro-4-(3-methyl-2-oxo-1,3-benzoxazol-5-yl)phenyl]ethyl]carbamoyl}-hexahydrofuro[3,4-b]pyrrole-1-carboxylate

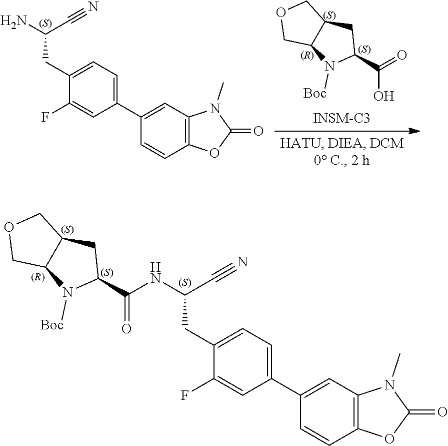

A solution of (2S,3aS,6aR)-1-(tert-butoxycarbonyl)-hexahydrofuro[3,4-b]pyrrole-2-carboxylic acid (60 mg, 0.23 mmol, 1.0 equiv) in DCM (5 mL) was treated with (2S)-2-amino-3-[2-fluoro-4-(3-methyl-2-oxo-1,3-benzoxazol-5-yl)phenyl]propanenitrile (87 mg, 0.28 mmol, 1.2 equiv), DIEA (90 mg, 0.69 mmol, 3.0 equiv) at room temperature. This was followed by the addition of HATU (106 mg, 0.28 mmol, 1.2 equiv) in portions at 0° C. The resulting mixture was stirred for additional 2 h at 0° C. Concentrated under reduced pressure, the residue was purified by silica gel column chromatography, eluted with PE/THF (1:1) to afford tert-butyl (2S,3aS,6aR)-2-{[(1S)-1-cyano-2-[2-fluoro-4-(3-methyl-2-oxo-1,3-benzoxazol-5-yl)phenyl]ethyl]carbamoyl}-hexahydrofuro[3,4-b]pyrrole-1-carboxylate (110 mg, 85.6%) as white solid. LCMS (ES, m/z): [M+H]⁺: 551.

### Synthesis of (2S,3aS,6aR)—N—((S)-1-cyano-2-(2-fluoro-4-(3-methyl-2-oxo-2,3-dihydrobenzo[d]oxazol-5-yl)phenyl)ethyl)hexahydro-1H-furo[3,4-b]pyrrole-2-carboxamide

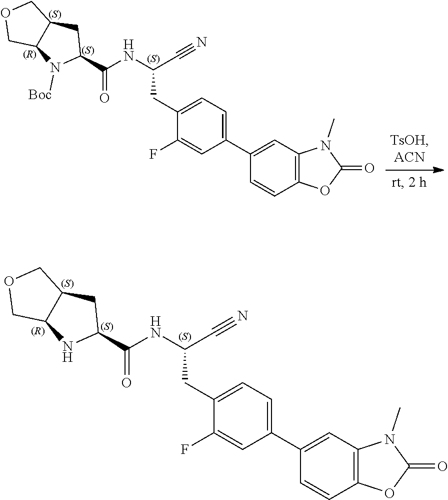

Into a 25 mL round-bottom flask were added tert-butyl (2S,3aS,6aR)-2-{[(1S)-1-cyano-2-[2-fluoro-4-(3-methyl-2-oxo-1,3-benzoxazol-5-yl)phenyl]ethyl]carbamoyl}-hexahydrofuro[3,4-b]pyrrole-1-carboxylate (110 mg, 0.20 mmol, 1.0 equiv), TsOH (103 mg, 0.60 mmol, 3.0 equiv) and ACN (3 mL) at room temperature. The resulting mixture was stirred for 2 h at room temperature. The residue was purified by reversed-phase flash chromatography with the following conditions: column, C18-120 g, mobile phase, MeCN in Water (0.1% NH₃·H₂O), 10% to 50% gradient in 10 min; detector, UV 254 nm. This resulted in (2S,3aS,6aR)—N-[(1S)-1-cyano-2-[2-fluoro-4-(3-methyl-2-oxo-1,3-benzoxazol-5-yl)phenyl]ethyl]-hexahydro-1H-furo[3,4-b]pyrrole-2-carboxamide (18.9 mg, 21%) as white solid.

### Analytical Data

LCMS (ES, m/z): [M+H]⁺: 451.2

¹H NMR (400 MHz, DMSO-d₆) δ 8.63 (d, J=8.4 Hz, 1H), 7.66 (s, 1H), 7.63-7.52 (m, 2H), 7.52-7.43 (m, 2H), 7.41 (d, J=8.3 Hz, 1H), 5.05 (q, J=8.0 Hz, 1H), 3.86-3.80 (m, 1H), 3.66 (d, J=9.3 Hz, 1H), 3.63-3.58 (m, 1H), 3.50-3.42 (m, 2H), 3.41 (s, 3H), 3.36 (dd, J=8.8, 3.5 Hz, 1H), 3.30-3.16 (m, 3H), 2.75-2.66 (m, 1H), 2.14-2.06 (m, 1H), 1.51-1.43 (m, 1H).

### Example 33. Synthesis of Compound 302: (2S,3aS,6aR)—N—((S)-1-cyano-2-(4′-cyano-3-fluoro-[1,1′-biphenyl]-4-yl)ethyl)hexahydro-1H-furo[3,4-b]pyrrole-2-carboxamide

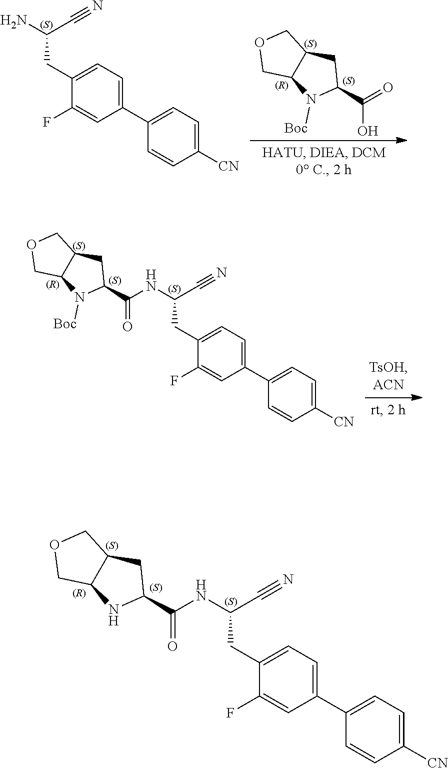

### Synthesis of tert-butyl (2S,3aS,6aR)-2-{[(1S)-1-cyano-2-{4′-cyano-3-fluoro-[1,1′-biphenyl]-4-yl}ethyl]carbamoyl}-hexahydrofuro[3,4-b]pyrrole-1-carboxylate

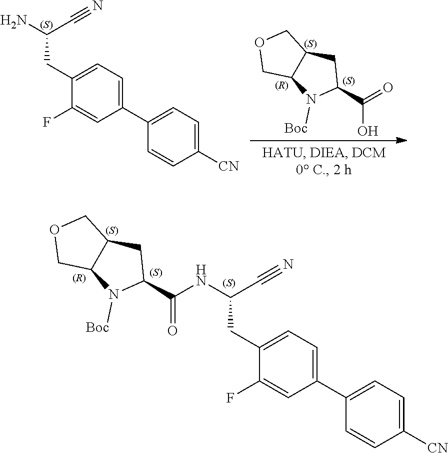

A solution of (2S,3aS,6aR)-1-(tert-butoxycarbonyl)-hexahydrofuro[3,4-b]pyrrole-2-carboxylic acid (60 mg, 0.23 mmol, 1.0 equiv) in DCM (5 mL) was treated with 4′-[(2S)-2-amino-2-cyanoethyl]-3′-fluoro-[1,1′-biphenyl]-4-carbonitrile (74 mg, 0.28 mmol, 1.2 equiv) and DIEA (90 mg, 0.69 mmol, 3.0 equiv) at room temperature. This was followed by the addition of HATU (106 mg, 0.28 mmol, 1.2 equiv) in portions at 0° C. The resulting mixture was stirred for 2 h at 0° C. Concentrated under reduced pressure to remove the solvent, the residue was purified by silica gel column chromatography, eluted with PE/THF (1:1) to afford tert-butyl (2S,3aS,6aR)-2-{[(1S)-1-cyano-2-{4′-cyano-3-fluoro-[1,1′-biphenyl]-4-yl}ethyl]carbamoyl}-hexahydrofuro[3,4-b]pyrrole-1-carboxylate (100 mg, 84.9%) as white solid. LCMS (ES, m/z): [M+H]⁺: 505.

### Synthesis of (2S,3aS,6aR)—N—((S)-1-cyano-2-(4′-cyano-3-fluoro-[1,1′-biphenyl]-4-yl)ethyl)hexahydro-1H-furo[3,4-b]pyrrole-2-carboxamide

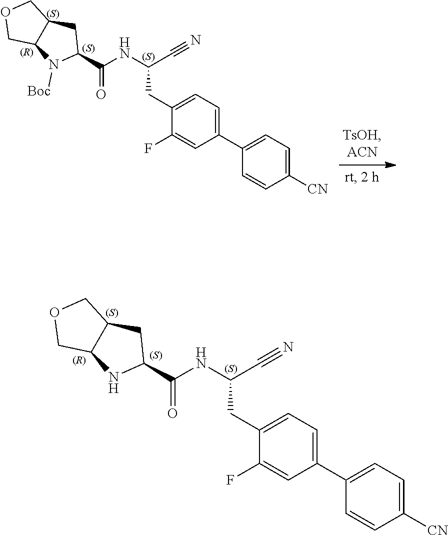

Into a 25 mL round-bottom flask were added tert-butyl (2S,3aS,6aR)-2-{[(1S)-1-cyano-2-{4′-cyano-3-fluoro-[1,1′-biphenyl]-4-yl}ethyl]carbamoyl}-hexahydrofuro[3,4-b]pyrrole-1-carboxylate (100 mg, 0.19 mmol, 1.0 equiv), TsOH (102 mg, 0.59 mmol, 3.0 equiv) and ACN (3 mL) at room temperature. The resulting mixture was stirred for 2 h at room temperature. The reaction solution was purified by reversed-phase flash chromatography with the following conditions: column, C18-120 g, mobile phase, MeCN in Water (0.1% NH₃·H₂O), 10% to 50% gradient in 10 min; detector, UV 254 nm. This resulted in (2S,3aS,6aR)—N—((S)-1-cyano-2-(4′-cyano-3-fluoro-[1, l′-biphenyl]-4-yl)ethyl)hexahydro-1H-furo[3,4-b]pyrrole-2-carboxamide (15.1 mg, 18.8%) as white solid.

### Analytical Data

LCMS (ES, m/z): [M+H]⁺: 405.2

¹H NMR (400 MHz, DMSO-d₆) δ 8.63 (d, J=8.4 Hz, 1H), 7.96-7.92 (m, 4H), 7.66 (dd, J=11.3, 1.8 Hz, 1H), 7.60 (dd, J=7.9, 1.8 Hz, 1H), 7.51 (t, J=7.9 Hz, 1H), 5.06 (q, J=7.9 Hz, 1H), 3.86-3.78 (m, 1H), 3.68-3.56 (m, 2H), 3.50-3.39 (m, 2H), 3.34-3.25 (m, 3H), 2.73-2.67 (m, 1H), 2.15-2.05 (m, 1H), 1.54-1.40 (m, 1H).

### Example 34. Compound 303: Synthesis of (2S,3aS,6aR)—N—((S)-1-cyano-2-(2-fluoro-4-(3-(methyl-d3)-2-oxo-2,3-dihydrobenzo[d]oxazol-5-yl)phenyl)ethyl)hexahydro-1H-furo[3,4-b]pyrrole-2-carboxamide

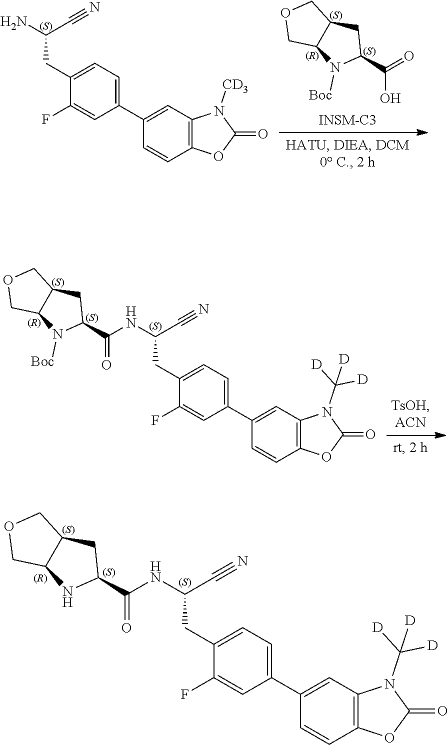

### Synthesis of tert-butyl (2S,3aS,6aR)-2-{[(1S)-1-cyano-2-{2-fluoro-4-[3-(2H3)methyl-2-oxo-1,3-benzoxazol-5-yl]phenyl}ethyl]carbamoyl}-hexahydrofuro[3,4-b]pyrrole-1-carboxylate

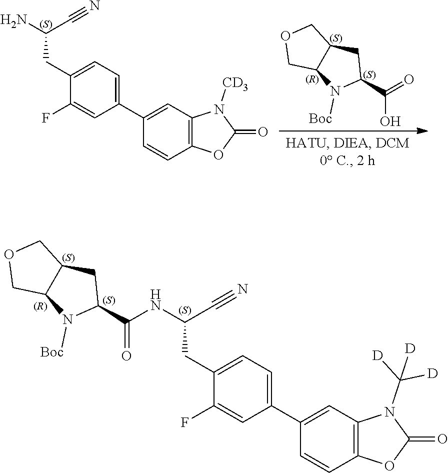

A solution of (2S)-2-amino-3-{2-fluoro-4-[3-(2H3)methyl-2-oxo-1,3-benzoxazol-5-yl]phenyl}propanenitrile (86 mg, 0.27 mmol, 1.0 equiv) in DCM (5 mL) was treated with (2S,3aS,6aR)-1-(tert-butoxycarbonyl)-hexahydrofuro[3,4-b]pyrrole-2-carboxylic acid (70 mg, 0.27 mmol, 1.0 equiv), DIEA (105 mg, 0.81 mmol, 3.0 equiv). This was followed by the addition of HATU (124 mg, 0.32 mmol, 1.2 equiv) in portions at 0° C. The resulting mixture was stirred for 2 h at 0° C. Concentrated under reduced pressure to remove the solvent, the residue was purified by silica gel column chromatography, eluted with PE/THF (1:1) to afford tert-butyl (2S,3aS,6aR)-2-{[(1S)-1-cyano-2-{2-fluoro-4-[3-(2H3)methyl-2-oxo-1,3-benzoxazol-5-yl]phenyl}ethyl]carbamoyl}-hexahydrofuro[3,4-b]pyrrole-1-carboxylate (85 mg, 56%) as white solid. LCMS (ES, m/z): [M+H]⁺: 554.

### Synthesis of (2S,3aS,6aR)—N-[(1S)-1-cyano-2-{2-fluoro-4-[3-(2H3)methyl-2-oxo-1,3-benzoxazol-5-yl]phenyl}ethyl]-hexahydro-1H-furo[3,4-b]pyrrole-2-carboxamide

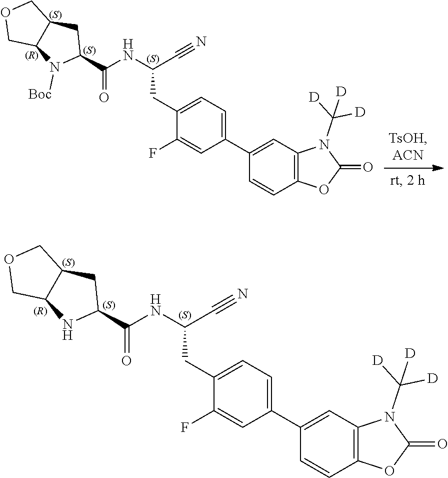

Into a 25 mL round-bottom flask were added tert-butyl (2S,3aS,6aR)-2-{[(1S)-1-cyano-2-{2-fluoro-4-[3-(2H3)methyl-2-oxo-1,3-benzoxazol-5-yl]phenyl}ethyl]carbamoyl}-hexahydrofuro[3,4-b]pyrrole-1-carboxylate (85 mg, 0.15 mmol, 1.0 equiv) in ACN (3 mL) and TsOH (79 mg, 0.46 mmol, 3.0 equiv) at room temperature. The resulting mixture was stirred for 3 h at room temperature. The reaction solution was purified by reversed-phase flash chromatography with the following conditions: column, C18 silica gel-120 g; mobile phase, MeCN in Water (0.1% NH₃·H₂O), 10% to 80% gradient in 10 min; detector, UV 254 nm. This resulted in (2S,3aS,6aR)—N-[(1S)-1-cyano-2-{2-fluoro-4-[3-(2H3)methyl-2-oxo-1,3-benzoxazol-5-yl]phenyl}ethyl]-hexahydro-1H-furo[3,4-b]pyrrole-2-carboxamide (19.0 mg, 27%) as white solid.

### Analytical Data

LCMS (ES, m/z): [M+H]⁺: 454.2

¹H NMR (400 MHz, DMSO-d₆) δ 8.63 (d, J=8.4 Hz, 1H), 7.66 (d, J=1.9 Hz, 1H), 7.59 (dd, J=11.6, 1.8 Hz, 1H), 7.54 (dd, J=7.9, 1.8 Hz, 1H), 7.51-7.43 (m, 2H), 7.41 (d, J=8.3 Hz, 1H), 5.05 (q, J=8.0 Hz, 1H), 3.83 (t, J=6.8 Hz, 1H), 3.70-3.54 (m, 2H), 3.49-3.41 (m, 2H), 3.36 (dd, J=8.8, 3.5 Hz, 1H), 3.32-3.17 (m, 3H), 2.74-2.66 (m, 1H), 2.14-2.06 (m, 1H), 1.54-1.44 (m, 1H).

### Example 35. Synthesis of Compound 304: (2S)—N-(1-cyano-2-(3-fluoro-4-(3-methyl-2-oxo-2,3-dihydrobenzo[d]oxazol-5-yl)phenyl)ethyl)-1,4-oxazepane-2-carboxamide

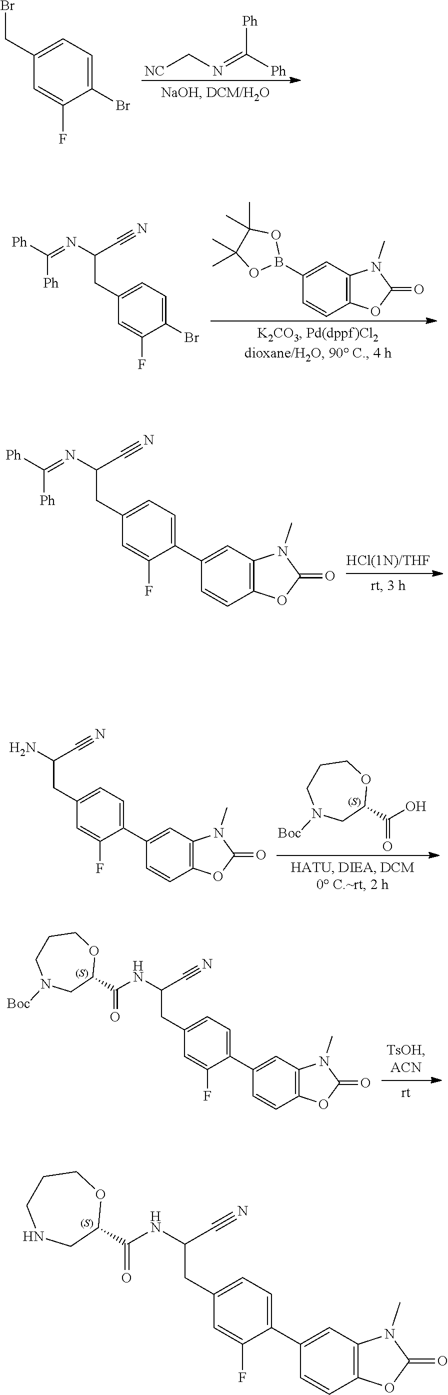

### Synthesis of 3-(4-bromo-3-fluorophenyl)-2-((diphenylmethylene)amino)propanenitrile

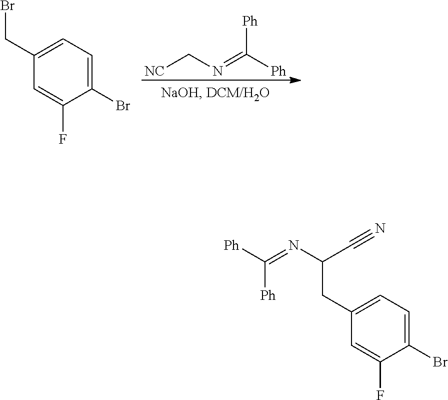 [SMILES: Fc1cc(CBr)ccc1Br ; N#CCN=C(c1ccccc1)c1ccccc1 ; N#CC(Cc1ccc(Br)c(F)c1)N=C(c1ccccc1)c1ccccc1]

To a 250 mL round bottom flask, 2-[(diphenylmethylidene)amino]acetonitrile (5 g, 22.70 mmol, 1.0 equiv), 1-bromo-4-(bromomethyl)-2-fluorobenzene (6.08 g, 22.70 mmol, 1.0 equiv), DCM (100 mL) and BnMe₃NCl (420 mg, 2.27 mmol, 0.1 equiv) were added in sequence at room temperature. This was followed by the addition of NaOH (1.82 g, 45.40 mmol, 2.0 equiv) in 10 mL H₂O. The mixture was stirred for 48 h at 40° C. The reaction was diluted with water (100 mL), extracted with dichloromethane (100 mL×1), dried over anhydrous sodium sulfate. Filtered and the filtrate was purified by silica gel column with ethyl acetate/Petroleum ether (5%). This result in 3-(4-bromo-3-fluorophenyl)-2-((diphenylmethylene)amino)propanenitrile (5.3 g, 57%) as yellow oil. LCMS (ES, m/z): [M+H]⁺: 407.

### Synthesis of 2-((diphenylmethylene)amino)-3-(3-fluoro-4-(3-methyl-2-oxo-2,3-dihydrobenzo[d]oxazol-5-yl)phenyl)propanenitrile

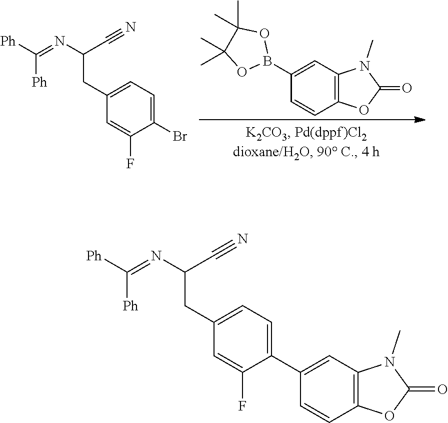 [SMILES: N#CC(Cc1ccc(Br)c(F)c1)N=C(c1ccccc1)c1ccccc1 ; Cn1c(=O)oc2ccc(B3OC(C)(C)C(C)(C)O3)cc21 ; Cn1c(=O)oc2ccc(-c3ccc(CC(C#N)N=C(c4ccccc4)c4ccccc4)cc3F)cc21]

To a 50 mL round bottom flask, 3-(4-bromo-3-fluorophenyl)-2-((diphenylmethylene)amino)propanenitrile (1 g, 2.45 mmol, 1.0 equiv), 3-methyl-5-(4,4,5,5-tetramethyl-1,3,2-dioxaborolan-2-yl)-1,3-benzoxazol-2-one (743 mg, 2.70 mmol, 1.1 equiv), K₂CO₃ (679 mg, 4.91 mmol, 2.0 equiv), dioxane (20 mL), H₂O (2 mL) and Pd(dppf)Cl₂ (90 mg, 0.12 mmol, 0.05 equiv) were added in sequence. The mixture was heated to 90° C. under nitrogen gas atmosphere and stirred for 4 h. The reaction was cooled to room temperature, concentrated to remove the solvent, the residue was purified by silica gel column with ethyl acetate/petroleum ether (1:3). This result in 2-((diphenylmethylene)amino)-3-(3-fluoro-4-(3-methyl-2-oxo-2,3-dihydrobenzo[d]oxazol-5-yl)phenyl)propanenitrile (1.0 g, 85%) as yellow solid. LCMS (ES, m/z): [M+H]⁺: 476.

### Synthesis of 2-amino-3-(3-fluoro-4-(3-methyl-2-oxo-2,3-dihydrobenzo[d]oxazol-5-yl)phenyl)propanenitrile

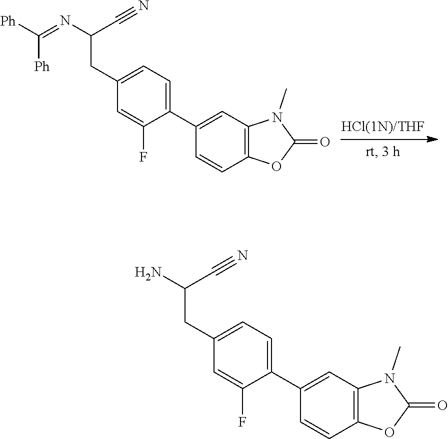 [SMILES: Cn1c(=O)oc2ccc(-c3ccc(CC(C#N)N=C(c4ccccc4)c4ccccc4)cc3F)cc21 ; Cn1c(=O)oc2ccc(-c3ccc(CC(N)C#N)cc3F)cc21]

To a solution of 2-((diphenylmethylene)amino)-3-(3-fluoro-4-(3-methyl-2-oxo-2,3-dihydrobenzo[d]oxazol-5-yl)phenyl)propanenitrile (1 g, 2.10 mmol, 1.0 equiv) in THF (50 mL), HCl (3N) (3 mL) was added dropwise at room temperature. The reaction was stirred for 3 h. The reaction was diluted with NaHCO₃(aq) (50 mL), extracted with ethyl acetate (50 mL×3). The combined organic phase was washed with brine (50 mL×1), dried over anhydrous sodium sulfate. Filtered and the filtrate was concentrated under reduced pressure. 2-amino-3-(3-fluoro-4-(3-methyl-2-oxo-2,3-dihydrobenzo[d]oxazol-5-yl)phenyl)propanenitrile (540 mg, 82%) was obtained as yellow oil and used to the next step without further purification. LCMS (ES, m/z): [M+H]⁺: 312.

### Synthesis of tert-butyl (2S)-2-({1-cyano-2-[3-fluoro-4-(3-methyl-2-oxo-1,3-benzoxazol-5-yl)phenyl]ethyl}carbamoyl)-1,4-oxazepane-4-carboxylate

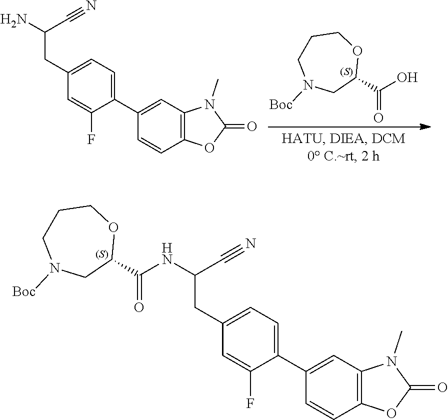

To a stirred mixture of 2-amino-3-(3-fluoro-4-(3-methyl-2-oxo-2,3-dihydrobenzo[d]oxazol-5-yl)phenyl)propanenitrile (112 mg, 0.36 mmol, 1.1 equiv) and (2S)-4-(tert-butoxycarbonyl)-1,4-oxazepane-2-carboxylic acid (80 mg, 0.33 mmol, 1.0 equiv), DIEA (126 mg, 0.98 mmol, 3 equiv) in DCM (5 mL) was added HATU (149 mg, 0.39 mmol, 1.2 equiv) at 0° C. The resulting mixture was stirred for 2 h at room temperature. The reaction was quenched with water (20 mL), extracted with dichloromethane (20 mL×2). The combined organic phase was dried over anhydrous sodium. Filtered and the filtrate was concentrated, the residue was purified by silica gel column chromatography, eluted with PE/THF (2:1) to afford tert-butyl (2S)-2-({1-cyano-2-[3-fluoro-4-(3-methyl-2-oxo-1,3-benzoxazol-5-yl)phenyl]ethyl}carbamoyl)-1,4-oxazepane-4-carboxylate (130 mg, 74%) as a light yellow oil. LCMS (ES, m/z): [M+H]⁺: 539.

### Synthesis of (2S)—N-(1-cyano-2-(3-fluoro-4-(3-methyl-2-oxo-2,3-dihydrobenzo[d]oxazol-5-yl)phenyl)ethyl)-1,4-oxazepane-2-carboxamide

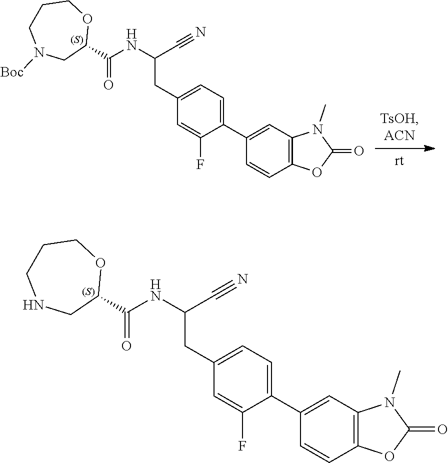

Into a 50 mL round-bottom flask were added tert-butyl (2S)-2-({1-cyano-2-[3-fluoro-4-(3-methyl-2-oxo-1,3-benzoxazol-5-yl)phenyl]ethyl}carbamoyl)-1,4-oxazepane-4-carboxylate (60 mg, 0.11 mmol, 1.0 equiv), ACN (3 mL) and TsOH·H₂O (64 mg, 0.33 mmol, 3.0 equiv) at room temperature. The resulting mixture was stirred for 2 h at room temperature. The reaction solution was purified by Prep-HPLC with the following conditions: Column, Atlantis Prep T3 OBD Column, 19*150 mm, 5 um; mobile phase, Water (0.05% NH₃·H₂O) and ACN (25% up to 50% in 10 min) to afford (2S)—N-(1-cyano-2-(3-fluoro-4-(3-methyl-2-oxo-2,3-dihydrobenzo[d]oxazol-5-yl)phenyl)ethyl)-1,4-oxazepane-2-carboxamide (22.6 mg, 46%) as a white solid.

### Analytical Data

LCMS (ES, m/z): [M+H]⁺: 439.

¹H NMR (400 MHz, DMSO-d₆) δ 8.62 (t, J=8.1 Hz, 1H), 7.53-7.48 (m, 1H), 7.42 (d, J=8.7 Hz, 2H), 7.34-7.17 (m, 3H), 5.12-4.98 (m, 1H), 4.05-3.80 (m, 2H), 3.78-3.54 (m, 1H), 3.38 (s, 3H), 3.27-3.19 (m, 2H), 3.14-3.01 (m, 1H), 2.84-2.54 (m, 3H), 1.79-1.70 (m, 2H).

### Example 36. Synthesis of Compound 305: (2S)—N-{1-cyano-2-[3-fluoro-5-(3-methyl-2-oxo-1,3-benzoxazol-5-yl)thiophen-2-yl]ethyl}-1,4-oxazepane-2-carboxamide

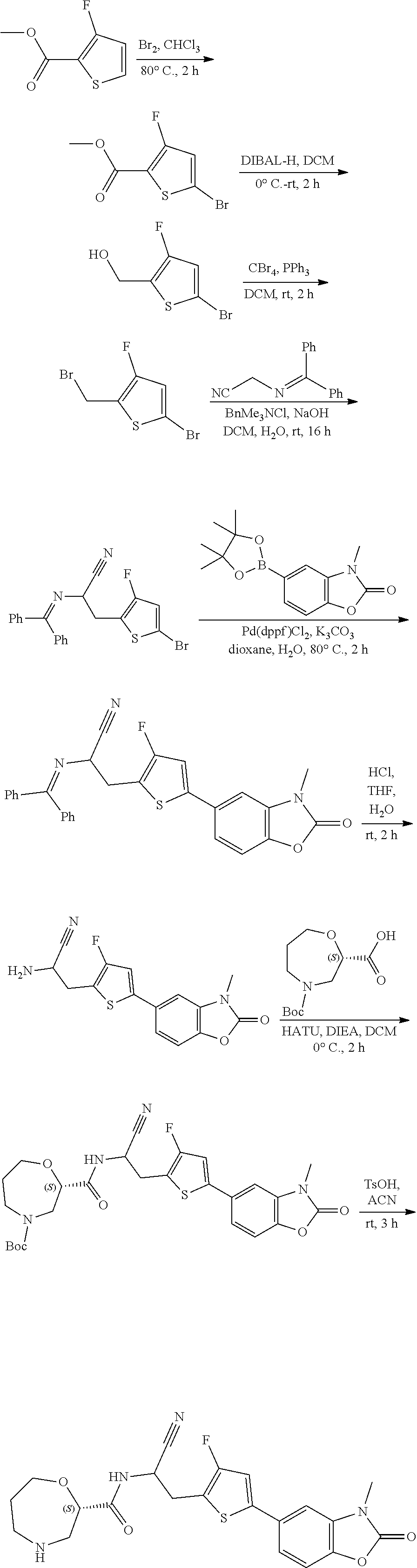

### Synthesis of methyl 5-bromo-3-fluorothiophene-2-carboxylate

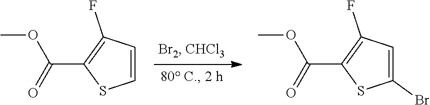

To a stirred solution of methyl 3-fluorothiophene-2-carboxylate (5 g, 31.22 mmol, 1.0 equiv) in CHCl₃ (70 mL) was added Br₂ (39.91 g, 249.75 mmol, 8.0 equiv). The resulting mixture was stirred for 3 h at 80° C. The mixture was allowed to cool down to room temperature. the reaction solution was poured into a saturated sodium thiosulfate solution (500 mL). The resulting mixture was diluted with CH₂Cl₂ (200 mL). The resulting mixture was filtered, the filter cake was washed with CH₂Cl₂ (3×50 mL). The filtrate was extracted with CH₂Cl₂ (3×100 mL). The combined organic layer was washed with brine (100 mL), dried over anhydrous Na₂SO₄. After filtration, the filtrate was concentrated under reduced pressure. The residue was purified by silica gel column chromatography, eluted with PE/EA (20:1) to afford methyl 5-bromo-3-fluorothiophene-2-carboxylate (3 g, 40%) as colorless oil. ¹H NMR (300 MHz, DMSO-d₆) δ 7.50 (s, 1H), 3.80 (s, 3H).

### Synthesis of (5-bromo-3-fluorothiophen-2-yl)methanol

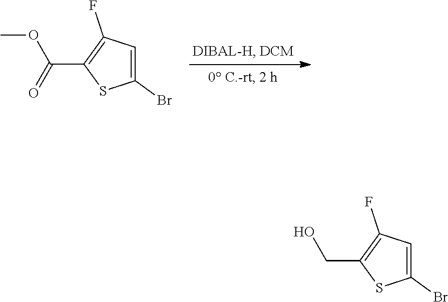

To a stirred solution of methyl 5-bromo-3-fluorothiophene-2-carboxylate (2 g, 8.37 mmol, 1.0 equiv) in DCM (40 mL) was added DIBAL-H (1 M in hexane) (25.1 mL, 25.10 mmol, 3.0 equiv) dropwise at 0° C. under nitrogen atmosphere. The resulting mixture was stirred for 2 h at room temperature under nitrogen atmosphere. The reaction was quenched with sodium potassium tartrate (aq.) (50 mL) at 0° C. The resulting mixture was extracted with CH₂Cl₂ (3×50 mL). The combined organic layer was washed with brine (50 mL), dried over anhydrous Na₂SO₄. After filtration, the filtrate was concentrated under reduced pressure. The residue was purified by silica gel column chromatography, eluted with PE/THF (12:1) to afford (5-bromo-3-fluorothiophen-2-yl)methanol (1.4 g, 79.2%) as colorless oil (no MS signal).

### Synthesis of 5-bromo-2-(bromomethyl)-3-fluorothiophene

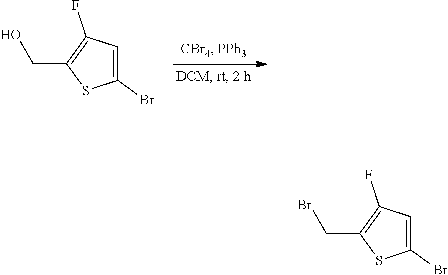 [SMILES: OCc1sc(Br)cc1F ; Fc1cc(Br)sc1CBr]

To a stirred solution of (5-bromo-3-fluorothiophen-2-yl)methanol (1.4 g, 6.63 mmol, 1.0 equiv) and CBr₄ (3.30 g, 9.95 mmol, 1.5 equiv) in DCM (40 mL) was added PPh₃ (2.61 g, 9.95 mmol, 1.5 equiv) at room temperature. The resulting mixture was stirred for 2 h at room temperature under nitrogen atmosphere. The resulting mixture was concentrated under reduced pressure. The residue was purified by silica gel column chromatography, eluted with PE/EA (99:1) to afford 5-bromo-2-(bromomethyl)-3-fluorothiophene (1.4 g, 77.0%) as colorless oil (no MS signal).

### Synthesis of 3-(5-bromo-3-fluorothiophen-2-yl)-2-[(diphenylmethylidene)amino]propanenitrile

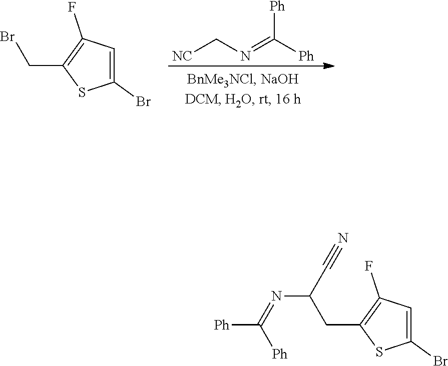 [SMILES: Fc1cc(Br)sc1CBr ; N#CCN=C(c1ccccc1)c1ccccc1 ; N#CC(Cc1sc(Br)cc1F)N=C(c1ccccc1)c1ccccc1]

To a stirred solution of 5-bromo-2-(bromomethyl)-3-fluorothiophene (1.3 g, 4.75 mmol, 1.0 equiv) and 2-[(diphenylmethylidene)amino]acetonitrile (1.05 g, 4.75 mmol, 1.0 equiv) in DCM (25 mL) and H₂O (2.5 mL) were added NaOH (0.38 g, 9.49 mmol, 2.0 equiv) and benzyltrimethylazanium chloride (0.09 g, 0.47 mmol, 0.1 equiv). The resulting mixture was stirred for 16 h at room temperature. The resulting mixture was diluted with water (30 mL), extracted with CH₂Cl₂ (2×50 mL). The combined organic layer was washed with brine (50 mL), dried over anhydrous Na₂SO₄. After filtration, the filtrate was concentrated under reduced pressure. The residue was purified by silica gel column chromatography, eluted with PE/EA (20:1) to afford 3-(5-bromo-3-fluorothiophen-2-yl)-2-[(diphenylmethylidene)amino]propanenitrile (1.2 g, 61.1%) as light yellow solid. LCMS (ES) [M+H]⁺ m/z: 413.

### Synthesis of 2-[(diphenylmethylidene)amino]-3-[3-fluoro-5-(3-methyl-2-oxo-1,3-benzoxazol-5-yl)thiophen-2-yl]propanenitrile

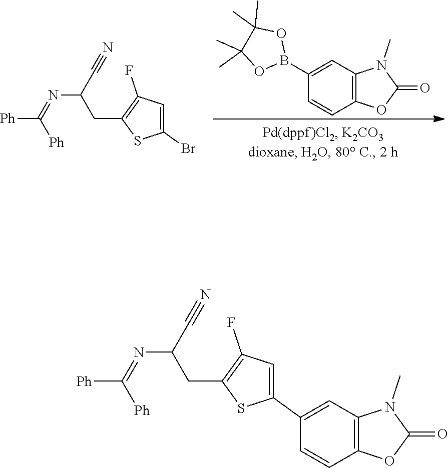 [SMILES: N#CC(Cc1sc(Br)cc1F)N=C(c1ccccc1)c1ccccc1 ; Cn1c(=O)oc2ccc(B3OC(C)(C)C(C)(C)O3)cc21 ; Cn1c(=O)oc2ccc(-c3cc(F)c(CC(C#N)N=C(c4ccccc4)c4ccccc4)s3)cc21]

To a stirred solution of 3-(5-bromo-3-fluorothiophen-2-yl)-2-[(diphenylmethylidene)amino]propanenitrile (1 g, 2.42 mmol, 1.0 equiv) and 3-methyl-5-(4,4,5,5-tetramethyl-1,3,2-dioxaborolan-2-yl)-1,3-benzoxazol-2-one (0.73 g, 2.66 mmol, 1.1 equiv) in dioxane (15 mL) and H₂O (1.5 mL) were added K₂CO₃ (0.67 g, 4.84 mmol, 2.0 equiv) and Pd(dppf)Cl₂ (0.18 g, 0.24 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (5:1) to afford 2-[(diphenylmethylidene)amino]-3-[3-fluoro-5-(3-methyl-2-oxo-1,3-benzoxazol-5-yl)thiophen-2-yl]propanenitrile (0.7 g, 60%) as light yellow solid. LCMS (ES) [M+H]⁺ m/z: 482.

### Synthesis of 2-amino-3-[3-fluoro-5-(3-methyl-2-oxo-1,3-benzoxazol-5-yl)thiophen-2-yl]propanenitrile

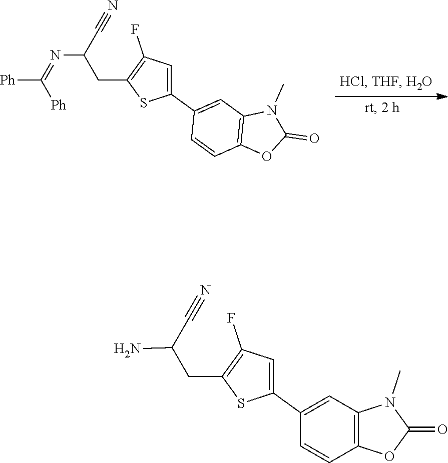 [SMILES: Cn1c(=O)oc2ccc(-c3cc(F)c(CC(C#N)N=C(c4ccccc4)c4ccccc4)s3)cc21 ; Cn1c(=O)oc2ccc(-c3cc(F)c(CC(N)C#N)s3)cc21]

To a stirred solution of 2-[(diphenylmethylidene)amino]-3-[3-fluoro-5-(3-methyl-2-oxo-1,3-benzoxazol-5-yl)thiophen-2-yl]propanenitrile (700 mg, 1.45 mmol, 1.0 equiv) in THF (35 mL) and H₂O (3.5 mL) was added HCl (1.75 mL). The resulting mixture was stirred for 2 h at room temperature. The resulting mixture was diluted with water (50 mL), extracted with diethyl ether (30 mL×1). The aqueous layer was basified to pH 12 with NaOH (aq.), extracted with CH₂Cl₂ (3×30 mL). The combined organic layer was washed with brine (30 mL), dried over anhydrous Na₂SO₄. After filtration, the filtrate was concentrated under reduced pressure. This resulted in 2-amino-3-[3-fluoro-5-(3-methyl-2-oxo-1,3-benzoxazol-5-yl)thiophen-2-yl]propanenitrile (370 mg, 80.2%) as light yellow solid. LCMS (ES) [M+H]⁺ m/z: 318.

### Synthesis of tert-butyl (2S)-2-({1-cyano-2-[3-fluoro-5-(3-methyl-2-oxo-1,3-benzoxazol-5-yl)thiophen-2-yl]ethyl}carbamoyl)-1,4-oxazepane-4-carboxylate

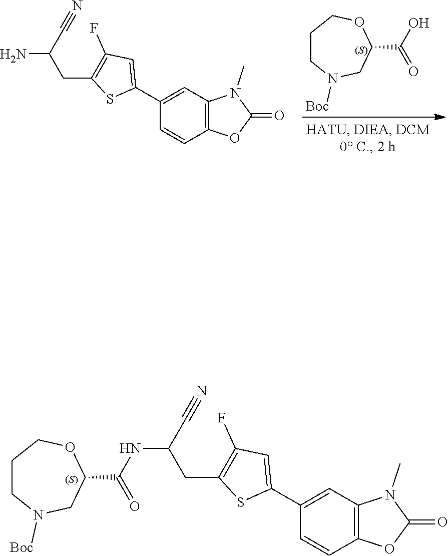

To a stirred solution of 2-amino-3-[3-fluoro-5-(3-methyl-2-oxo-1,3-benzoxazol-5-yl)thiophen-2-yl]propanenitrile (99 mg, 0.31 mmol, 1.1 equiv) and (2S)-4-(tert-butoxycarbonyl)-1,4-oxazepane-2-carboxylic acid (70 mg, 0.28 mmol, 1.0 equiv) in DCM (2 mL) were added DIEA (110 mg, 0.86 mmol, 3.0 equiv) and HATU (130 mg, 0.34 mmol, 1.2 equiv) in portions at 0° C. under nitrogen atmosphere. The resulting mixture was stirred for 2 h at 0° C. The residue was purified by silica gel column chromatography, eluted with PE/EA (2:1) to afford tert-butyl (2S)-2-({1-cyano-2-[3-fluoro-5-(3-methyl-2-oxo-1,3-benzoxazol-5-yl)thiophen-2-yl]ethyl}carbamoyl)-1,4-oxazepane-4-carboxylate (100 mg, 64.3%) as white solid. LCMS (ES) [M+H]⁺ m/z: 545.

### Synthesis of (2S)—N-{1-cyano-2-[3-fluoro-5-(3-methyl-2-oxo-1,3-benzoxazol-5-yl)thiophen-2-yl]ethyl}-1,4-oxazepane-2-carboxamide

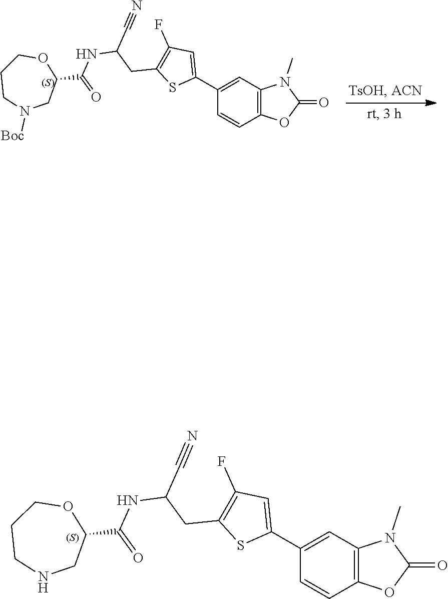

To a stirred solution of tert-butyl (2S)-2-({1-cyano-2-[3-fluoro-5-(3-methyl-2-oxo-1,3-benzoxazol-5-yl)thiophen-2-yl]ethyl}carbamoyl)-1,4-oxazepane-4-carboxylate (90 mg, 0.16 mmol, 1.0 equiv) in ACN (3 mL) was added TsOH (85 mg, 0.50 mmol, 3.0 equiv). The resulting mixture was stirred for 3 h at room temperature. The reaction solution was purified by Prep-HPLC with the following conditions: Column, XBridge Prep C18 OBD Column, 19*150 mm, 5 um; mobile phase, Water (10 mmol/L NH₄HCO₃) and ACN (30% Phase B up to 40% in 7 min); Detector, UV 254 nm. This resulted in (2S)—N-{1-cyano-2-[3-fluoro-5-(3-methyl-2-oxo-1,3-benzoxazol-5-yl)thiophen-2-yl]ethyl}-1,4-oxazepane-2-carboxamide (30 mg, 40.8%) as white solid. LCMS (ES) [M+H]⁺ m/z: 445. ¹H NMR (300 MHz, DMSO-d₆) δ 8.74 (dd, J=8.4, 4.1 Hz, 1H), 7.57-7.55 (d, J=2.7 Hz, 1H), 7.47 (d, J=1.5 Hz, 1H), 7.40-7.34 (m, 2H), 5.06-4.88 (m, 1H), 4.08-3.87 (m, 2H), 3.80-3.70 (m, 1H), 3.47-3.35 (m, 4H), 3.30-3.24 (m, 1H), 3.20-3.06 (m, 1H), 2.88-2.59 (m, 3H), 1.82-1.70 (m, 2H).

### Example 37. Synthesis of Compound 158: (S)—N-(1-cyano-2-(4-(3-methyl-2-oxo-2,3-dihydrobenzo[d]oxazol-5-yl)phenyl)ethyl)cyclohexanecarboxamide

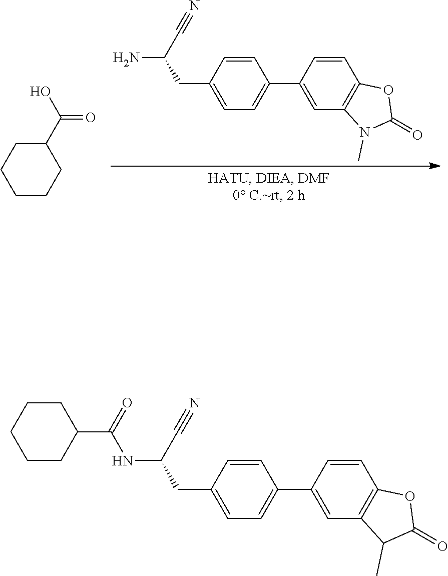 [SMILES: O=C(O)C1CCCCC1 ; Cn1c(=O)oc2ccc(-c3ccc(C[C@H](N)C#N)cc3)cc21 ; CC1C(=O)Oc2ccc(-c3ccc(C[C@@H](C#N)NC(=O)C4CCCCC4)cc3)cc21]

### Synthesis of (S)—N-(1-cyano-2-(4-(3-methyl-2-oxo-2,3-dihydrobenzo[d]oxazol-5-yl)phenyl)ethyl)cyclohexanecarboxamide

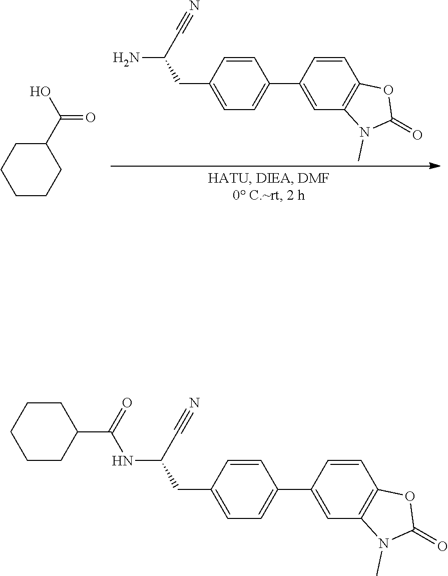 [SMILES: O=C(O)C1CCCCC1 ; Cn1c(=O)oc2ccc(-c3ccc(C[C@H](N)C#N)cc3)cc21 ; Cn1c(=O)oc2ccc(-c3ccc(C[C@@H](C#N)NC(=O)C4CCCCC4)cc3)cc21]

To a stirred mixture of cyclohexanecarboxylic acid (30 mg, 0.23 mmol, 1.0 equiv) and (2S)-2-amino-3-[4-(3-methyl-2-oxo-1,3-benzoxazol-5-yl)phenyl]propanenitrile (82 mg, 0.28 mmol, 1.2 equiv), DIEA (91 mg, 0.70 mmol, 3.0 equiv) in DMF (3 mL) was added HATU (107 mg, 0.28 mmol, 1.2 equiv) at 0° C. The resulting mixture was stirred for 2 h at room temperature. The crude product was purified by Prep-HPLC with the following conditions: Column, Atlantis Prep T3 OBD Column, 19*150 mm, 5 um; mobile phase, Water (0.05% NH₃·H₂O) and ACN (25% up to 50% in 10 min) to afford(S)—N-(1-cyano-2-(4-(3-methyl-2-oxo-2,3-dihydrobenzo[d]oxazol-5-yl)phenyl)ethyl)cyclohexanecarboxamide (22.1 mg, 23%) as white solid.

### Analytical Data

LCMS (ES, m/z): [M+H]⁺: 404.

¹H NMR (400 MHz, DMSO-d₆) δ ¹H NMR (400 MHz, DMSO-d₆) δ 8.57 (d, J=7.8 Hz, 1H), 7.67 (d, J=8.0 Hz, 2H), 7.58 (d, J=1.7 Hz, 1H), 7.46-7.36 (m, 4H), 4.96 (q, J=7.8 Hz, 1H), 3.41 (s, 3H), 3.19-3.04 (m, 2H), 2.16-2.06 (m, 1H), 1.76-1.57 (m, 5H), 1.35-1.14 (m, 5H).

### Example 38. Synthesis of Compound 194: (2S,3aS,6aS)—N-[(1S)-1-cyano-2-{4′-cyano-3-fluoro-[1,1′-biphenyl]-4-yl}ethyl]-octahydrocyclopenta[b]pyrrole-2-carboxamide

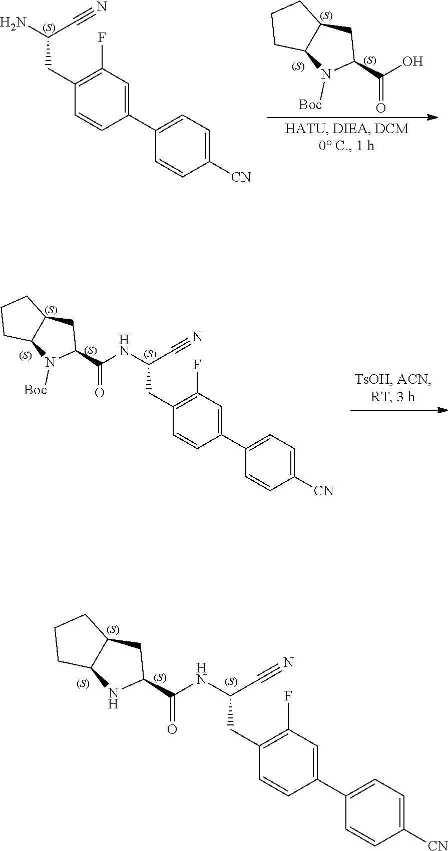

### Synthesis of tert-butyl (2S,3aS,6aS)-2-{[(1S)-1-cyano-2-{4′-cyano-3-fluoro-[1,1′-biphenyl]-4-yl}ethyl]carbamoyl}-hexahydro-2H-cyclopenta[b]pyrrole-1-carboxylate

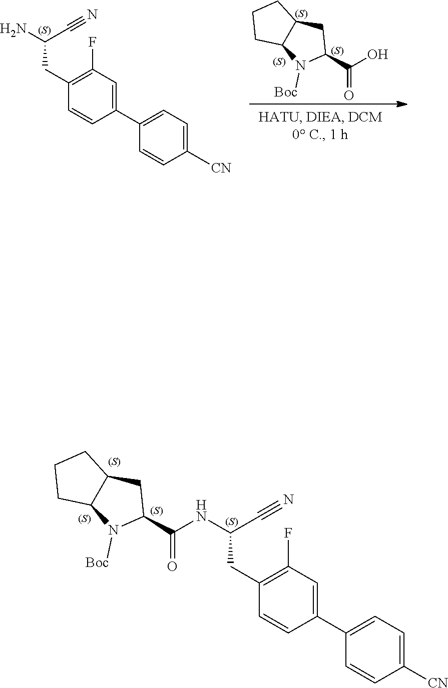

To a stirred solution of 4′-[(2S)-2-amino-2-cyanoethyl]-3′-fluoro-[1,1′-biphenyl]-4-carbonitrile (74 mg, 0.281 mmol, 1.1 equiv) and (2S,3aS,6aS)-1-(tert-butoxycarbonyl)-hexahydro-2H-cyclopenta[b]pyrrole-2-carboxylic acid (65 mg, 0.255 mmol, 1.0 equiv) in DCM (2 mL) was added DIEA (65 mg, 0.510 mmol, 2.0 equiv). To the above mixture was added HATU (116 mg, 0.306 mmol, 1.2 equiv) in portions at 0° C. The resulting mixture was stirred for additional 1 h at 0° C. The resulting mixture was concentrated under reduced pressure. The residue was purified by silica gel column chromatography, eluted with PE/THF (3:1) to afford tert-butyl (2S,3aS,6aS)-2-{[(1S)-1-cyano-2-{4′-cyano-3-fluoro-[1,1′-biphenyl]-4-yl}ethyl]carbamoyl}-hexahydro-2H-cyclopenta[b]pyrrole-1-carboxylate (90 mg, 70.34%) as a colorless oil. LCMS (ES) [M+1]⁺ m/z: 503

### Synthesis of (2S,3aS,6aS)—N-[(1S)-1-cyano-2-{4′-cyano-3-fluoro-[1,1′-biphenyl]-4-yl}ethyl]-octahydrocyclopenta[b]pyrrole-2-carboxamide

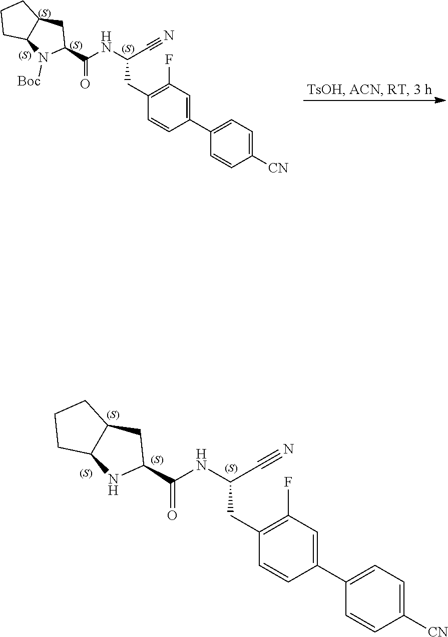

To a stirred solution of tert-butyl (2S,3aS,6aS)-2-{[(1S)-1-cyano-2-{4′-cyano-3-fluoro-[1,1′-biphenyl]-4-yl}ethyl]carbamoyl}-hexahydro-2H-cyclopenta[b]pyrrole-1-carboxylate (90 mg, 0.179 mmol, 1.0 equiv) in ACN (2 mL) was added TsOH (92 mg, 0.537 mmol, 3.0 equiv). The resulting mixture was stirred for 3 h at room temperature. The crude product was purified by Prep-HPLC with the following conditions (Column, XBridge Prep C18 OBD Column, 19*150 mm 5 um; mobile phase, Water (10 MMOL/L NH₄HCO₃) and ACN (30% PhaseB up to 40% in 7 min); Detector, UV, 220 nm.) to afford (2S,3aS,6aS)—N-[(1S)-1-cyano-2-{4′-cyano-3-fluoro-[1,1′-biphenyl]-4-yl}ethyl]-octahydrocyclopenta[b]pyrrole-2-carboxamide (25 mg, 34.69%) as a white solid.

¹H NMR (400 MHz, DMSO-d₆) δ 8.50 (d, J=8.6 Hz, 1H), 7.98-7.89 (m, 4H), 7.69-7.62 (m, 1H), 7.59 (dd, J=8.1, 1.8 Hz, 1H), 7.50 (t, J=7.9 Hz, 1H), 5.11 (q, J=8.0 Hz, 1H), 3.58 (t, J=6.7 Hz, 1H), 3.40 (t, J=8.1 Hz, 1H), 3.29 (d, J=7.9 Hz, 2H), 2.99 (s, 1H), 2.46-2.36 (m, 1H), 2.05 (dt, J=12.3, 8.0 Hz, 1H), 1.62-1.49 (m, 2H), 1.49-1.35 (m, 3H), 1.26-1.14 (m, 1H), 1.02-0.90 (m, 1H). LCMS (ES) [M+1]⁺ m/z: 403

### Example 39. Synthesis of Compound 306: (2S,3aS,6aS)—N-[(1S)-1-cyano-2-[2-fluoro-4-(3-methyl-2-oxo-1,3-benzoxazol-5-yl)phenyl]ethyl]-octahydrocyclopenta[b]pyrrole-2-carboxamide

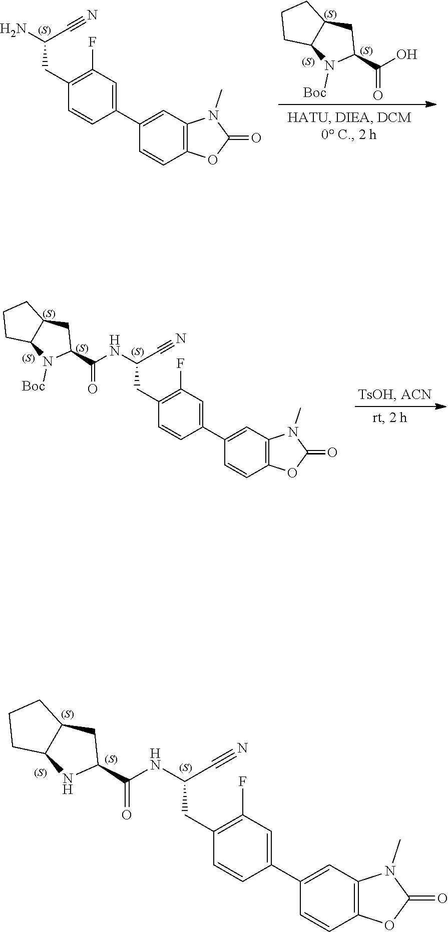

### Synthesis of tert-butyl (2S)-2-{[(1S)-1-cyano-2-{4′-[(4-methylpiperazin-1-yl)methyl]-[1,1′-biphenyl]-4-yl}ethyl]carbamoyl}-1,4-oxazepane-4-carboxylate

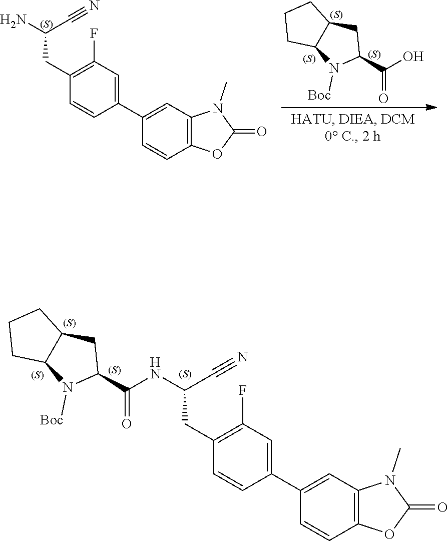

A solution of (2S,3aS,6aS)-1-(tert-butoxycarbonyl)-hexahydro-2H-cyclopenta[b]pyrrole-2-carboxylic acid (60 mg, 0.23 mmol, 1.0 equiv) in DCM (5 mL) was treated with (2S)-2-amino-3-[2-fluoro-4-(3-methyl-2-oxo-1,3-benzoxazol-5-yl)phenyl]propanenitrile (88 mg, 0.28 mmol, 1.2 equiv), DIEA (91 mg, 0.70 mmol, 3.0 equiv) at room temperature. This was followed by the addition of HATU (107 mg, 0.28 mmol, 1.2 equiv) in portions at 0° C. The resulting mixture was stirred for 2 h at 0° C. Concentrated under reduced pressure to remove the solvent. The residue was purified by silica gel column chromatography, eluted with PE/THF (1:1) to afford tert-butyl (2S,3aS,6aS)-2-{[(1S)-1-cyano-2-[2-fluoro-4-(3-methyl-2-oxo-1,3-benzoxazol-5-yl)phenyl]ethyl]carbamoyl}-hexahydro-2H-cyclopenta[b]pyrrole-1-carboxylate (102 mg, 79%) as white solid. LCMS (ES, m/z): [M+H]⁺: 549.

### Synthesis of (2S,3aS,6aS)—N-[(1S)-1-cyano-2-[2-fluoro-4-(3-methyl-2-oxo-1,3-benzoxazol-5-yl)phenyl]ethyl]-octahydrocyclopenta[b]pyrrole-2-carboxamide

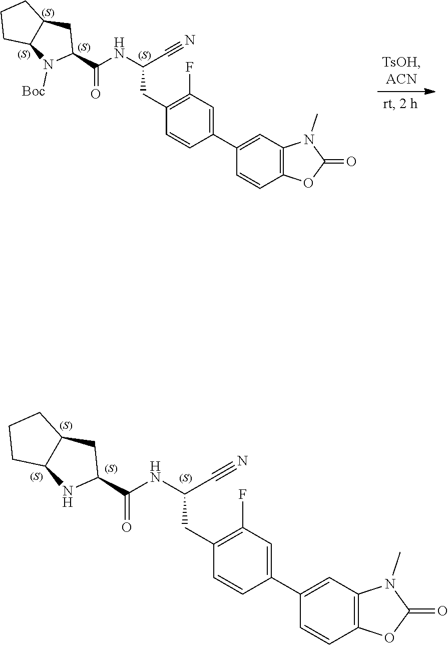

Into a 25 mL round-bottom flask were added tert-butyl (2S,3aS,6aS)-2-{[(1S)-1-cyano-2-[2-fluoro-4-(3-methyl-2-oxo-1,3-benzoxazol-5-yl)phenyl]ethyl]carbamoyl}-hexahydro-2H-cyclopenta[b]pyrrole-1-carboxylate (102 mg, 0.18 mmol, 1.0 equiv), TsOH (96 mg, 0.55 mmol, 3.0 equiv) and ACN (3 mL) at room temperature. The resulting mixture was stirred for 2 h at room temperature. The reaction was purified by reversed-phase flash chromatography with the following conditions: column, C18-120 g, mobile phase, MeCN in Water (0.1% NH₃·H₂O), 10% to 80% gradient in 10 min; detector, UV 254 nm. This resulted in (2S,3aS,6aS)—N-[(1S)-1-cyano-2-[2-fluoro-4-(3-methyl-2-oxo-1,3-benzoxazol-5-yl)phenyl]ethyl]-octahydrocyclopenta[b]pyrrole-2-carboxamide (15.8 mg, 19%) as white solid.

### Analytical Data

LCMS (ES, m/z): [M+H]⁺: 449.2

¹H NMR (400 MHz, DMSO-d₆) δ 8.51 (d, J=8.6 Hz, 1H), 7.65 (d, J=1.8 Hz, 1H), 7.62-7.51 (m, 2H), 7.50-7.44 (m, 2H), 7.41 (d, J=8.3 Hz, 1H), 5.10 (q, J=8.1 Hz, 1H), 3.63-3.54 (m, 1H), 3.43-3.39 (m, 4H), 3.30-3.20 (m, 2H), 2.44-2.40 (m, 1H), 2.13-2.01 (m, 1H), 1.67-1.49 (m, 2H), 1.49-1.34 (m, 3H), 1.26-1.16 (m, 1H), 0.99 (dt, J=12.2, 8.1 Hz, 1H).

### Example 40. Synthesis of Compound 307: (2S,3aS,6aS)—N-[(1S)-1-cyano-2-[4-(3-methyl-2-oxo-1,3-benzoxazol-5-yl)phenyl]ethyl]-octahydrocyclopenta[b]pyrrole-2-carboxamide

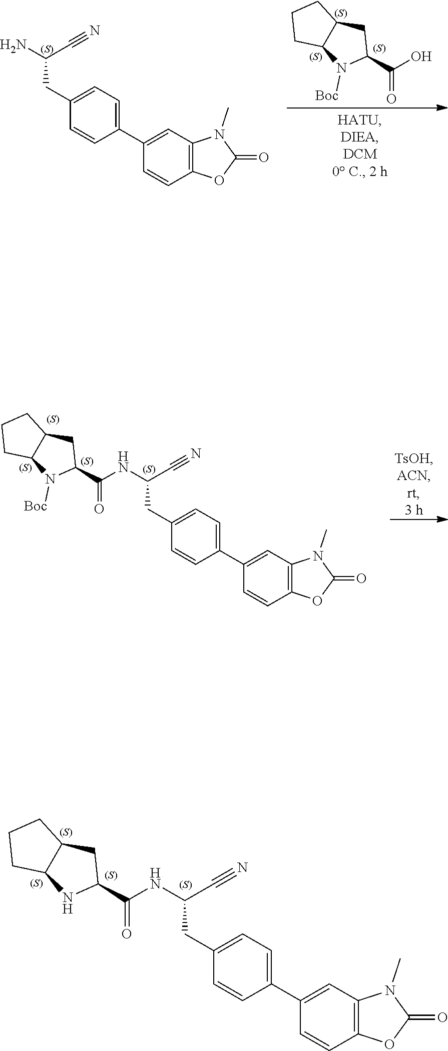

### Synthesis of tert-butyl (2S,3aS,6aS)-2-{[(1S)-1-cyano-2-[4-(3-methyl-2-oxo-1,3-benzoxazol-5-yl)phenyl]ethyl]carbamoyl}-hexahydro-2H-cyclopenta[b]pyrrole-1-carboxylate

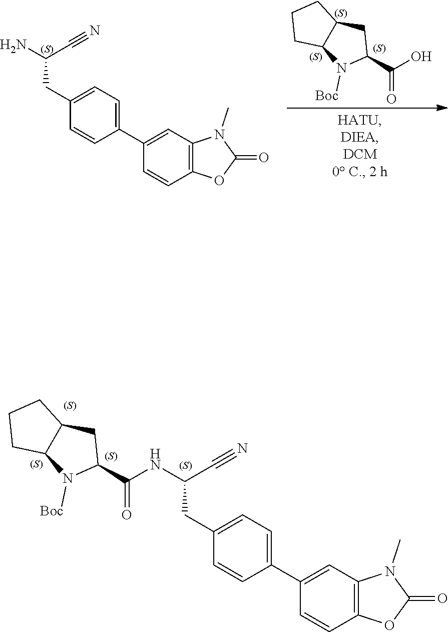

To a stirred solution of (2S)-2-amino-3-[4-(3-methyl-2-oxo-1,3-benzoxazol-5-yl)phenyl]propanenitrile (70 mg, 0.24 mmol, 1.0 equiv) and (2S,3aS,6aS)-1-(tert-butoxycarbonyl)-hexahydro-2H-cyclopenta[b]pyrrole-2-carboxylic acid (60 mg, 0.24 mmol, 1.0 equiv) in DCM (1 mL) were added DIEA (92 mg, 0.72 mmol, 3.0 equiv) and HATU (108 mg, 0.29 mmol, 1.2 equiv) in portions at 0° C. under nitrogen gas atmosphere. The resulting mixture was stirred for 2 h at 0° C. Concentrated under reduced pressure, the residue was purified by silica gel column chromatography, eluted with PE/THF (2:1) to afford tert-butyl (2S,3aS,6aS)-2-{[(1S)-1-cyano-2-[4-(3-methyl-2-oxo-1,3-benzoxazol-5-yl)phenyl]ethyl]carbamoyl}-hexahydro-2H-cyclopenta[b]pyrrole-1-carboxylate (90 mg, 71%) as white solid. LCMS (ES) [M+1]⁺ m/z: 531.

### Synthesis of (2S,3aS,6aS)—N-[(1S)-1-cyano-2-[4-(3-methyl-2-oxo-1,3-benzoxazol-5-yl)phenyl]ethyl]-octahydrocyclopenta[b]pyrrole-2-carboxamide

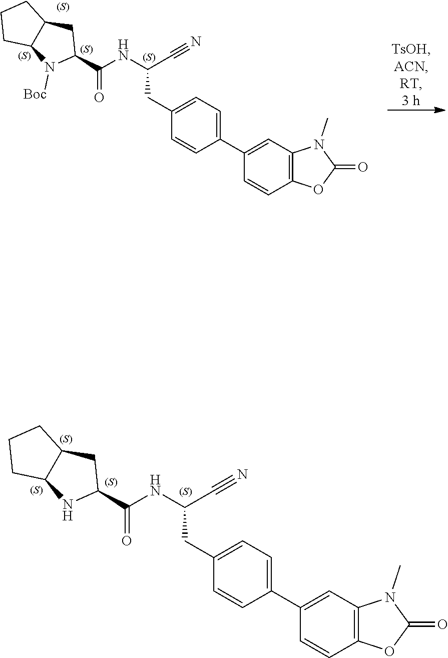

To a stirred solution of tert-butyl (2S,3aS,6aS)-2-{[(1S)-1-cyano-2-[4-(3-methyl-2-oxo-1,3-benzoxazol-5-yl)phenyl]ethyl]carbamoyl}-hexahydro-2H-cyclopenta[b]pyrrole-1-carboxylate (80 mg, 0.15 mmol, 1.0 equiv) in ACN (2 mL) was added TsOH (77 mg, 0.45 mmol, 3.0 equiv). The resulting mixture was stirred for 3 h at room temperature. The reaction solution was purified by Prep-HPLC with the following conditions Column, XBridge Prep C18 OBD Column, 19*150 mm, 5 um; mobile phase, Water (10 mmol/L NH₄HCO₃) and ACN (30% PhaseB up to 40% in 7 min); Detector, UV 254 nm. This resulted in (2S,3aS,6aS)—N-[(1S)-1-cyano-2-[4-(3-methyl-2-oxo-1,3-benzoxazol-5-yl)phenyl]ethyl]-octahydrocyclopenta[b]pyrrole-2-carboxamide (30 mg, 46.2%) as white solid. LCMS (ES) [M+1]⁺ m/z: 431.

¹H NMR (300 MHz, DMSO-d₆) δ 8.39 (d, J=8.5 Hz, 1H), 7.64 (d, J=7.9 Hz, 2H), 7.56 (s, 1H), 7.43-7.35 (m, 4H), 5.07 (q, J=7.9 Hz, 1H), 3.57 (t, J=7.9 Hz, 1H), 3.40-3.36 (m, 4H), 3.22 (d, J=7.8 Hz, 2H), 2.98 (brs, 1H), 2.43-2.38 (m, 1H), 2.09-2.00 (m, 1H), 1.67-1.33 (m, 5H), 1.27-1.10 (m, 1H), 1.00-0.90 (m, 1H).

### Example 41. Synthesis of Compound 159: (2S)—N-{1-cyano-2-[2,5-difluoro-4-(3-methyl-2-oxo-1,3-benzoxazol-5-yl)phenyl]ethyl}-1,4-oxazepane-2-carboxamide

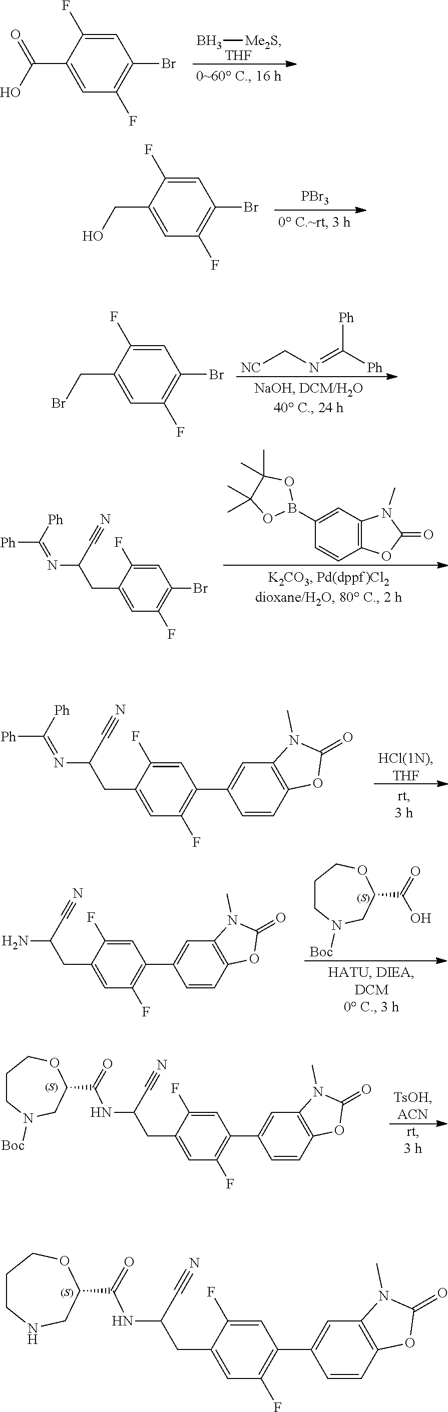

### Synthesis of (4-bromo-2,5-difluorophenyl)methanol

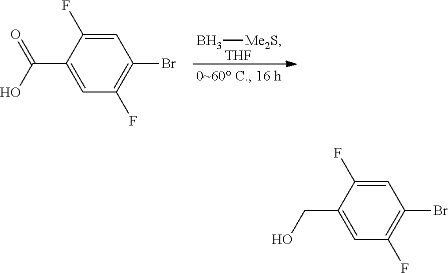

To a stirred solution of 4-bromo-2,5-difluorobenzoic acid (5 g, 21.10 mmol, 1.0 equiv) in THF (50 mL) was added BH₃-Me₂S (1 M) (63.3 mL, 63.30 mmol, 3.0 equiv) dropwise at 0° C. The resulting mixture was stirred for 16 h at 60° C. The reaction was cooled to room temperature, quenched with MeOH at room temperature. The resulting mixture was concentrated under reduced pressure. The residue was purified by silica gel column chromatography, eluted with PE/EA (5:1) to afford (4-bromo-2,5-difluorophenyl)methanol (4 g, 85%) as an off-white solid (no LCMS signal).

### Synthesis of 1-bromo-4-(bromomethyl)-2,5-difluorobenzene

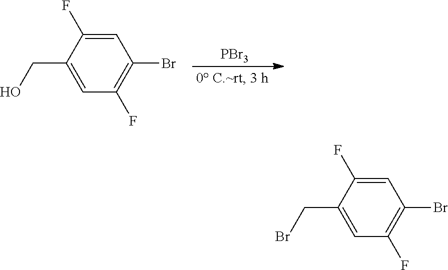

To a stirred solution of (4-bromo-2,5-difluorophenyl)methanol (2 g, 8.97 mmol, 1.0 equiv) in DCM (30 mL) was added PBr₃ (1.21 g, 4.48 mmol, 0.5 equiv) at 0° C. The resulting mixture was stirred for 3 h at room temperature. The reaction was quenched with water (20 mL), extracted with CH₂Cl₂ (50 mL×1). The combined organic layer was washed with brine (50 mL), dried over anhydrous Na₂SO₄. After filtration, the filtrate was concentrated under reduced pressure. This resulted in 1-bromo-4-(bromomethyl)-2,5-difluorobenzene (2 g, 78%) as light yellow oil (no LCMS signal).

### Synthesis of 3-(4-bromo-2,5-difluorophenyl)-2-((diphenylmethylene)amino)propanenitrile

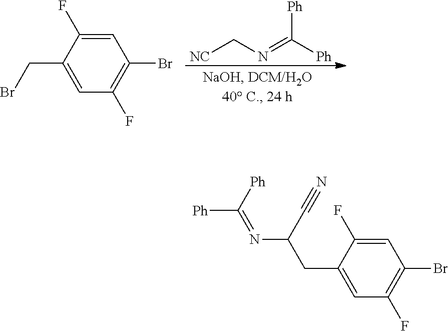

To a solution of 1-bromo-4-(bromomethyl)-2,5-difluorobenzene (2 g, 7.00 mmol, 1.0 equiv) in THF (30 mL) was added 2-[(diphenylmethylidene)amino]acetonitrile (1.54 g, 7.00 mmol, 1.0 equiv), benzyltrimethylazanium chloride (0.13 g, 0.70 mmol, 0.1 equiv), NaOH (0.56 g, 13.99 mmol, 2.0 equiv) in H₂O (3 mL). The mixture was stirred for 4 h at 60° C. The reaction was diluted with water (50 mL), extracted with CH₂Cl₂ (3×100 mL). The combined organic layer was washed with brine (30 mL), dried over anhydrous Na₂SO₄. After filtration, the filtrate was concentrated under reduced pressure. The residue was purified by silica gel column chromatography, eluted with PE/EA (5:1) to afford 3-(4-bromo-2,5-difluorophenyl)-2-[(diphenylmethylidene)amino]propanenitrile (2.8 g, 94%) as light yellow solid. LCMS (ES, m/z): [M+H]⁺: 425.

### Synthesis of 3-[2,5-difluoro-4-(3-methyl-2-oxo-1,3-benzoxazol-5-yl)phenyl]-2-[(diphenylmethylidene)amino]propanenitrile

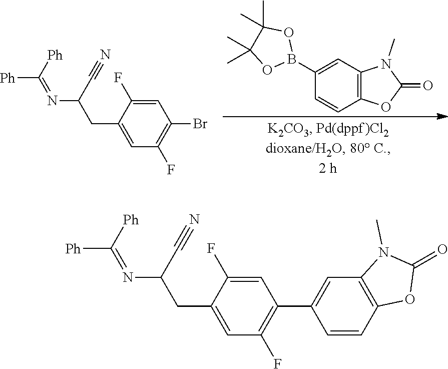 [SMILES: N#CC(Cc1cc(F)c(Br)cc1F)N=C(c1ccccc1)c1ccccc1 ; Cn1c(=O)oc2ccc(B3OC(C)(C)C(C)(C)O3)cc21 ; Cn1c(=O)oc2ccc(-c3cc(F)c(CC(C#N)N=C(c4ccccc4)c4ccccc4)cc3F)cc21]

To a solution of 3-(4-bromo-2,5-difluorophenyl)-2-[(diphenylmethylidene)amino]propanenitrile (1.0 g, 2.35 mmol, 1.0 equiv), 3-methyl-5-(4,4,5,5-tetramethyl-1,3,2-dioxaborolan-2-yl)-1,3-benzoxazol-2-one (0.8 g, 2.82 mmol, 1.2 equiv) in 1,4-dioxane (10 mL) and H₂O (1 mL) were added K₂CO₃ (0.7 g, 4.70 mmol, 2.0 equiv) and Pd(dppf)Cl₂ (0.2 g, 0.23 mmol, 0.1 equiv) was stirred for 2 h at 80° C. under nitrogen atmosphere. The reaction was cooled to room temperature, concentrated under reduced pressure. The residue was purified by silica gel column chromatography, eluted with PE/THF (1:1) to afford 3-[2,5-difluoro-4-(3-methyl-2-oxo-1,3-benzoxazol-5-yl)phenyl]-2-[(diphenylmethylidene)amino]propanenitrile (800 mg, 69%) as yellow oil. LCMS (ES, m/z): [M+H]⁺: 494.

### Synthesis of tert-butyl (2S)-2-({1-cyano-2-[2,5-difluoro-4-(3-methyl-2-oxo-1,3-benzoxazol-5-yl)phenyl]ethyl}carbamoyl)-1,4-oxazepane-4-carboxylate

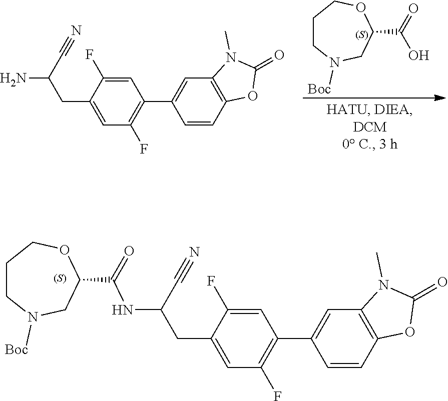

To a stirred mixture of 2-amino-3-[2,5-difluoro-4-(3-methyl-2-oxo-1,3-benzoxazol-5-yl)phenyl]propanenitrile (89 mg, 0.27 mmol, 1.1 equiv) and DIEA (95 mg, 0.73 mmol, 3.0 equiv) in DCM (5 mL) was added HATU (112 mg, 0.29 mmol, 1.2 equiv) in portions at 0° C. The resulting mixture was stirred for additional 3 h at 0° C. The residue was purified by silica gel column chromatography, eluted with PE/THF (1:1) to afford tert-butyl (2S)-2-({1-cyano-2-[2,5-difluoro-4-(3-methyl-2-oxo-1,3-benzoxazol-5-yl)phenyl]ethyl}carbamoyl)-1,4-oxazepane-4-carboxylate (100 mg, 73.4%) as white solid. LCMS (ES, m/z): [M+H]⁺: 557.

### Synthesis of (2S)—N-{1-cyano-2-[2,5-difluoro-4-(3-methyl-2-oxo-1,3-benzoxazol-5-yl)phenyl]ethyl}-1,4-oxazepane-2-carboxamide

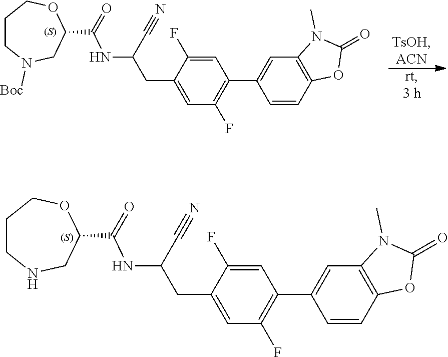

Into a 25 mL round-bottom flask were added tert-butyl (2S)-2-({1-cyano-2-[2,5-difluoro-4-(3-methyl-2-oxo-1,3-benzoxazol-5-yl)phenyl]ethyl}carbamoyl)-1,4-oxazepane-4-carboxylate (100 mg, 0.180 mmol, 1.0 equiv) in ACN (3 mL) and TsOH (93 mg, 0.54 mmol, 3.0 equiv) at room temperature. The resulting mixture was stirred for 3 h at room temperature. The reaction solution was purified by reversed-phase flash chromatography with the following conditions: column, C18 silica gel-120 g; mobile phase, MeCN in Water (0.1% NH₃·H₂O), 10% to 80% gradient in 10 min; detector, UV 254 nm. This resulted in (2S)—N-{1-cyano-2-[2,5-difluoro-4-(3-methyl-2-oxo-1,3-benzoxazol-5-yl)phenyl]ethyl}-1,4-oxazepane-2-carboxamide (16.0 mg, 20%) as white solid.

### Analytical Data

LCMS (ES, m/z): [M+H]⁺: 457.3

¹H NMR (400 MHz, DMSO-d₆) δ 8.72 (dd, J=10.9, 8.5 Hz, 1H), 7.52-7.37 (m, 4H), 7.35-7.30 (m, 1H), 5.15-5.00 (m, 1H), 4.03-3.94 (m, 1H), 3.92-3.82 (m, 1H), 3.77-3.68 (m, 1H), 3.39 (s, 3H), 3.30-3.20 (m, 2H), 3.16-3.02 (m, 1H), 2.85-2.68 (m, 2H), 2.66-2.53 (m, 2H), 1.80-1.67 (m, 2H).

### Example 42. Synthesis of Compound 246: N-[(1S)-1-cyano-2-[4-(3-methyl-2-oxo-1,3-benzoxazol-5-yl)phenyl]ethyl]-6-azaspiro[3.4]octane-8-carboxamide

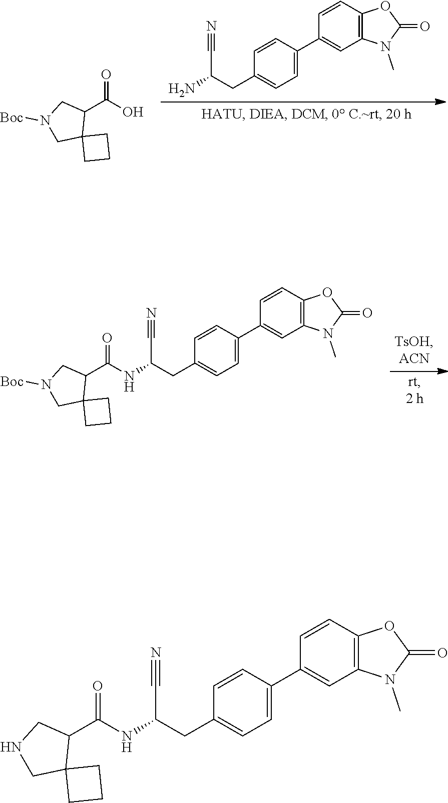 [SMILES: CC(C)(C)OC(=O)N1CC(C(=O)O)C2(CCC2)C1 ; Cn1c(=O)oc2ccc(-c3ccc(C[C@H](N)C#N)cc3)cc21 ; Cn1c(=O)oc2ccc(-c3ccc(C[C@@H](C#N)NC(=O)C4CN(C(=O)OC(C)(C)C)CC45CCC5)cc3)cc21 ; Cn1c(=O)oc2ccc(-c3ccc(C[C@@H](C#N)NC(=O)C4CNCC45CCC5)cc3)cc21]

### Synthesis of tert-butyl 8-{[(1S)-1-cyano-2-[4-(3-methyl-2-oxo-1,3-benzoxazol-5-yl)phenyl]ethyl]carbamoyl}-6-azaspiro[3.4]octane-6-carboxylate

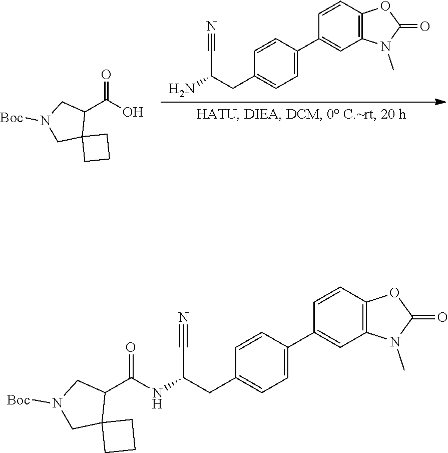 [SMILES: CC(C)(C)OC(=O)N1CC(C(=O)O)C2(CCC2)C1 ; Cn1c(=O)oc2ccc(-c3ccc(C[C@H](N)C#N)cc3)cc21 ; Cn1c(=O)oc2ccc(-c3ccc(C[C@@H](C#N)NC(=O)C4CN(C(=O)OC(C)(C)C)CC45CCC5)cc3)cc21]

To a stirred mixture of 6-(tert-butoxycarbonyl)-6-azaspiro[3.4]octane-8-carboxylic acid (87 mg, 0.34 mmol, 1 equiv), (2S)-2-amino-3-[4-(3-methyl-2-oxo-1,3-benzoxazol-5-yl)phenyl]propanenitrile (100 mg, 0.34 mmol, 1.00 equiv) and DIEA (132 mg, 1.02 mmol, 3 equiv) in DCM (5 mL) were added HATU (155 mg, 0.41 mmol, 1.2 equiv) in portions at 0° C. The resulting mixture was stirred for additional 20 h at room temperature. The residue was purified by silica gel column chromatography, eluted with PE/THF (1:1) to afford tert-butyl 8-{[(1S)-1-cyano-2-[4-(3-methyl-2-oxo-1,3-benzoxazol-5-yl)phenyl]ethyl]carbamoyl}-6-azaspiro[3.4]octane-6-carboxylate (120 mg, 66.34%) as a light yellow oil. LCMS (ES, m/z): [M+H]⁺: 531.

### Synthesis of N-[(1S)-1-cyano-2-[4-(3-methyl-2-oxo-1,3-benzoxazol-5-yl)phenyl]ethyl]-6-azaspiro[3.4]octane-8-carboxamide

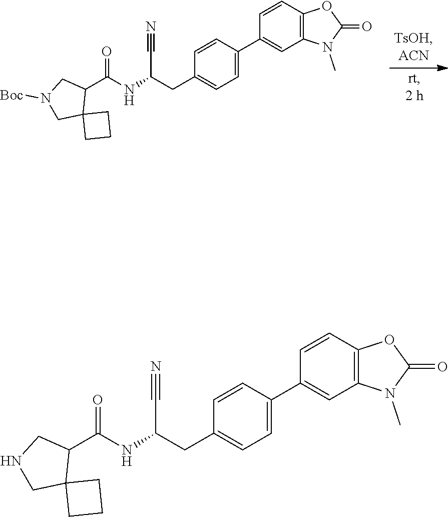 [SMILES: Cn1c(=O)oc2ccc(-c3ccc(C[C@@H](C#N)NC(=O)C4CN(C(=O)OC(C)(C)C)CC45CCC5)cc3)cc21 ; Cn1c(=O)oc2ccc(-c3ccc(C[C@@H](C#N)NC(=O)C4CNCC45CCC5)cc3)cc21]

Into a 50 mL round-bottom flask were added tert-butyl 8-{[(1S)-1-cyano-2-[4-(3-methyl-2-oxo-1,3-benzoxazol-5-yl)phenyl]ethyl]carbamoyl}-6-azaspiro[3.4]octane-6-carboxylate (120 mg, 0.23 mmol, 1 equiv), TsOH·H₂O (129 mg, 0.68 mmol, 3 equiv) and ACN (3 mL) at room temperature. The resulting mixture was stirred for additional 2 h at room temperature. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in N-[(1S)-1-cyano-2-[4-(3-methyl-2-oxo-1,3-benzoxazol-5-yl)phenyl]ethyl]-6-azaspiro[3.4]octane-8-carboxamide (20 mg, 20.54%) as a white solid.

### Analytical Data

LCMS (ES, m/z): [M+H]⁺: 431.

¹H NMR (400 MHz, DMSO-d₆) ¹H NMR (400 MHz, DMSO-d₆) δ 9.15-8.65 (m, 1H), 7.74-7.63 (m, 2H), 7.61-7.53 (m, 1H), 7.50-7.36 (m, 4H), 5.33-4.89 (m, 1H), 3.41 (d, J=3.1 Hz, 3H), 3.30-3.05 (m, 3H), 2.97-2.63 (m, 3H), 2.49-2.41 (m, 1H), 2.08-1.91 (m, 1H), 1.91-1.51 (m, 4H), 1.48-1.34 (m, 1H).

### Example 43. Synthesis of 249 and 249A: (2S,3S)—N-[(1S)-1-cyano-2-[4-(3-methyl-2-oxo-1,3-benzoxazol-5-yl)phenyl]ethyl]-2-methylpiperidine-3-carboxamide; (2R,3R)—N-[(1S)-1-cyano-2-[4-(3-methyl-2-oxo-1,3-benzoxazol-5-yl)phenyl]ethyl]-2-methylpiperidine-3-carboxamide

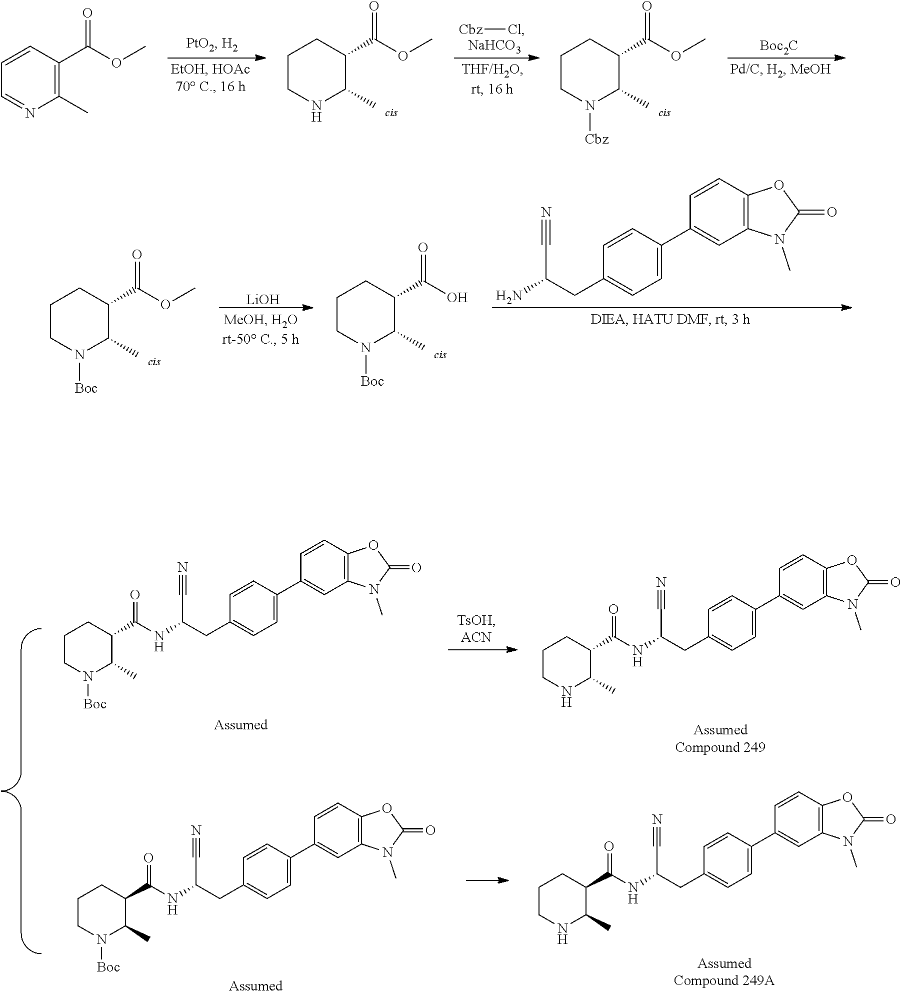

### Synthesis of methyl (2S,3S)-2-methylpiperidine-3-carboxylate

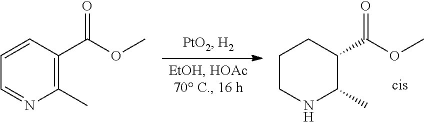

A solution of methyl 2-methylpyridine-3-carboxylate (500 mg, 3.30 mmol, 1.0 equiv) and PtO₂ (50 mg, 0.02 mmol, 0.01 equiv, 10%) in EtOH (3 mL) HOAc (3 mL) was stirred for 16 h at 70° C., 30 atm under nitrogen atmosphere. The resulting mixture was filtered, the filter cake was washed with EtOH (3×10 mL). The filtrate was concentrated under reduced pressure. This resulted in methyl (2S,3S)-2-methylpiperidine-3-carboxylate (500 mg, crude) as a white oil. LCMS (ES, m/z): [M+H]⁺: 158

### Synthesis of 1-benzyl 3-methyl (2S,3S)-2-methylpiperidine-1,3-dicarboxylate

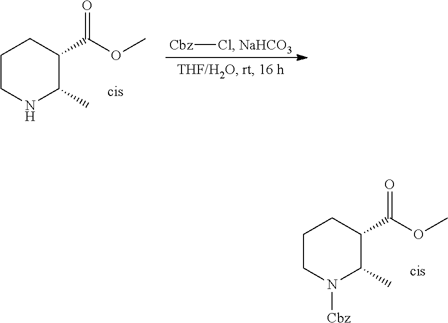

To a stirred solution of methyl (2S,3S)-2-methylpiperidine-3-carboxylate (500 mg, 3.180 mmol, 1.0 equiv) in THF (5 mL) and NaHCO₃ (2.7 g, 31.80 mmol, 10.0 equiv) in H₂O (5 mL) were added CbzCl (108 mg, 0.63 mmol, 0.2 equiv) in portions at 0° C. The resulting mixture was stirred for additional 16 h at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EA (1:1) to afford 1-benzyl 3-methyl (2S,3S)-2-methylpiperidine-1,3-dicarboxylate (300 mg, 32.3%) as a white oil. LCMS (ES, m/z): [M+H]⁺: 292.

### Synthesis of 1-tert-butyl 3-methyl (2S,3S)-2-methylpiperidine-1,3-dicarboxylate

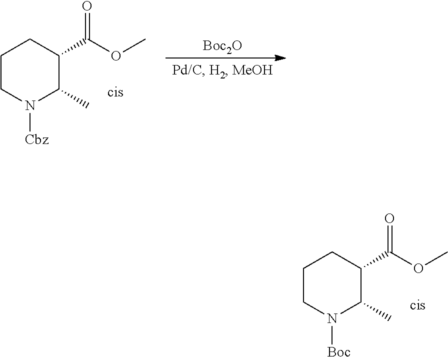 [SMILES: "Boc2O" ; COC(=O)[C@H]1CCCN(C(=O)OCc2ccccc2)[C@H]1C ; COC(=O)[C@H]1CCCN(C(=O)OC(C)(C)C)[C@H]1C]

A solution of 1-benzyl 3-methyl (2S,3S)-2-methylpiperidine-1,3-dicarboxylate (350 mg, 1.20 mmol, 1.0 equiv) and Boc₂O (393 mg, 1.80 mmol, 1.5 equiv), Pd/C (5 mg, 0.005 mmol, 10%) in MeOH (5 mL) was stirred for 3 h at room temperature under hydrogen atmosphere. The resulting mixture was filtered, the filter cake was washed with THF (3×5 mL). The filtrate was concentrated under reduced pressure. This resulted in 1-tert-butyl 3-methyl (2S,3S)-2-methylpiperidine-1,3-dicarboxylate (370 mg, crude) as a white oil.

LCMS (ES, m/z): [M+H]⁺: 258

### Synthesis of (2S,3S)-1-(tert-butoxycarbonyl)-2-methylpiperidine-3-carboxylic acid

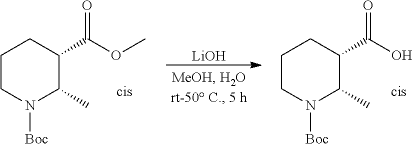

Into a 50 mL round-bottom flask were added 1-tert-butyl 3-methyl (2S,3S)-2-methylpiperidine-1,3-dicarboxylate (370 mg, 1.43 mmol, 1.0 equiv) and lithium hydroxide (69 mg, 2.87 mmol, 2.0 equiv) in MeOH (0.6 mL) and H₂O (0.2 mL) at room temperature. The resulting mixture was stirred for additional 5h at room temperature. The mixture was acidified to pH 5 with citric acid. The resulting mixture was extracted with EtOAc (3×30 mL). The combined organic layers were washed with brine (3×30 mL), dried over anhydrous Na₂SO₄. After filtration, the filtrate was concentrated under reduced pressure. This resulted in (2S,3S)-1-(tert-butoxycarbonyl)-2-methylpiperidine-3-carboxylic acid (180 mg, 51.4%) as a white oil. LCMS (ES, m/z): [M+H]⁺: 244

### Synthesis of tert-butyl (2R,3R)-3-{[(1S)-1-cyano-2-[4-(3-methyl-2-oxo-1,3-benzoxazol-5-yl)phenyl]ethyl]carbamoyl}-2-methylpiperidine-1-carboxylate; tert-butyl (2S,3S)-3-{[(1S)-1-cyano-2-[4-(3-methyl-2-oxo-1,3-benzoxazol-5-yl)phenyl]ethyl]carbamoyl}-2-methylpiperidine-1-carboxylate

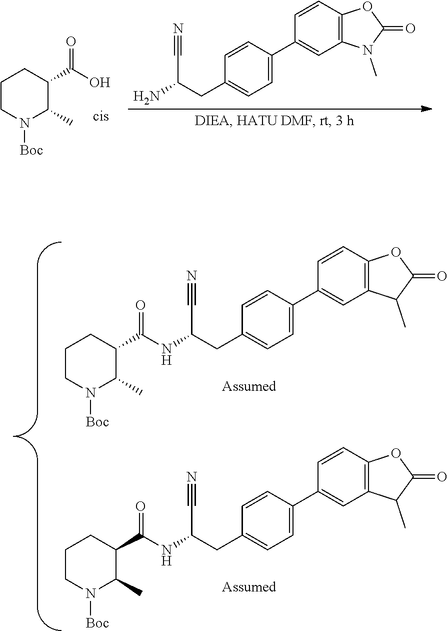 [SMILES: C[C@H]1[C@@H](C(=O)O)CCCN1C(=O)OC(C)(C)C ; Cn1c(=O)oc2ccc(-c3ccc(C[C@H](N)C#N)cc3)cc21 ; CC1C(=O)Oc2ccc(-c3ccc(C[C@@H](C#N)NC(=O)[C@H]4CCCN(C(=O)OC(C)(C)C)[C@H]4C)cc3)cc21 ; CC1C(=O)Oc2ccc(-c3ccc(C[C@@H](C#N)NC(=O)[C@@H]4CCCN(C(=O)OC(C)(C)C)[C@@H]4C)cc3)cc21]

To a stirred solution of (2S,3S)-1-(tert-butoxycarbonyl)-2-methylpiperidine-3-carboxylic acid (180 mg, 0.74 mmol, 2.5 equiv) DMF (5 mL) and DIEA (115 mg, 0.88 mmol, 3.0 equiv) in were added HATU (135 mg, 0.35 mmol, 1.2 equiv) in portions at 0° C. The resulting mixture was stirred for additional 3 h at 0° C. The resulting mixture was extracted with EtOAc (3×10 mL). The combined organic layers were washed with brine (3×10 mL), dried over anhydrous Na₂SO₄. After filtration, the filtrate was concentrated under reduced pressure. This resulted in tert-butyl (2R,3R)-3-{[(1S)-1-cyano-2-[4-(3-methyl-2-oxo-1,3-benzoxazol-5-yl)phenyl]ethyl]carbamoyl}-2-methylpiperidine-1-carboxylate (110 mg, 71.67%); tert-butyl (2S,3S)-3-{[(1S)-1-cyano-2-[4-(3-methyl-2-oxo-1,3-benzoxazol-5-yl)phenyl]ethyl]carbamoyl}-2-methylpiperidine-1-carboxylate (110 mg, 71.67%) as a white solid. LCMS (ES, m/z): [M+H]⁺: 519

### Synthesis of (2S,3S)—N-[(1S)-1-cyano-2-[4-(3-methyl-2-oxo-1,3-benzoxazol-5-yl)phenyl]ethyl]-2-methylpiperidine-3-carboxamide

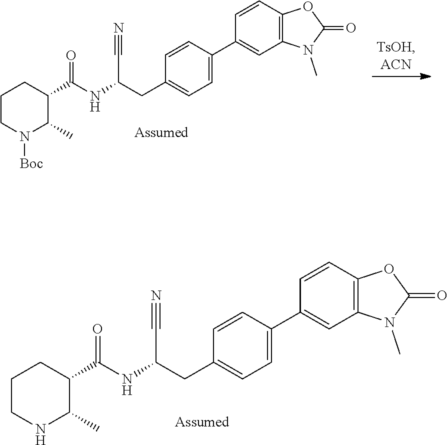 [SMILES: C[C@H]1[C@@H](C(=O)N[C@H](C#N)Cc2ccc(-c3ccc4oc(=O)n(C)c4c3)cc2)CCCN1C(=O)OC(C)(C)C ; C[C@@H]1NCCC[C@@H]1C(=O)N[C@H](C#N)Cc1ccc(-c2ccc3oc(=O)n(C)c3c2)cc1]

Into a 50 mL round-bottom flask were added tert-butyl (2S,3S)-3-{[(1S)-1-cyano-2-[4-(3-methyl-2-oxo-1,3-benzoxazol-5-yl)phenyl]ethyl]carbamoyl}-2-methylpiperidine-1-carboxylate (110 mg, 0.21 mmol, 1.0 equiv) in ACN (3 mL) and TsOH (109 mg, 0.63 mmol, 3.0 equiv) at room temperature. The resulting mixture was stirred for additional 3 h at room temperature. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in (2S,3S)—N-[(1S)-1-cyano-2-[4-(3-methyl-2-oxo-1,3-benzoxazol-5-yl)phenyl]ethyl]-2-methylpiperidine-3-carboxamide (17.3 mg, 19.49%) as a white solid.

### Analytical Data

LCMS (ES, m/z): [M+H]⁺: 419.2.

¹H NMR (400 MHz, DMSO-d₆) δ 9.29 (d, J=7.8 Hz, 1H), 7.68 (d, J=8.1 Hz, 2H), 7.58 (d, J=1.5 Hz, 1H), 7.46-7.36 (m, 4H), 5.11-5.01 (m, 1H), 3.41 (s, 3H), 3.24 (dd, J=13.6, 6.4 Hz, 1H), 3.13 (dd, J=13.6, 8.7 Hz, 1H), 2.88-2.81 (m, 1H), 2.75-2.61 (m, 1H), 2.44 (d, J=10.4 Hz, 1H), 2.34 (s, 1H), 2.27-2.21 (m, 2H), 1.71-1.62 (m, 1H), 1.56-1.50 (m, 1H), 1.38-1.29 (m, 1H), 1.26-1.17 (m, 1H), 0.86 (d, J=6.6 Hz, 3H).

### Synthesis of (2R,3R)—N-[(1S)-1-cyano-2-[4-(3-methyl-2-oxo-1,3-benzoxazol-5-yl)phenyl]ethyl]-2-methylpiperidine-3-carboxamide

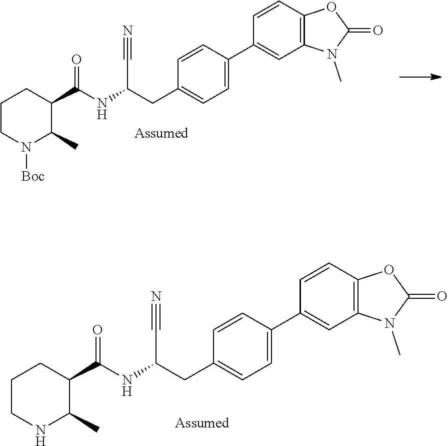 [SMILES: C[C@@H]1[C@H](C(=O)N[C@H](C#N)Cc2ccc(-c3ccc4oc(=O)n(C)c4c3)cc2)CCCN1C(=O)OC(C)(C)C ; C[C@H]1NCCC[C@H]1C(=O)N[C@H](C#N)Cc1ccc(-c2ccc3oc(=O)n(C)c3c2)cc1]

Into a 50 mL round-bottom flask were added tert-butyl (2R,3R)-3-{[(1S)-1-cyano-2-[4-(3-methyl-2-oxo-1,3-benzoxazol-5-yl)phenyl]ethyl]carbamoyl}-2-methylpiperidine-1-carboxylate (110 mg, 0.21 mmol, 1.0 equiv) in ACN (3 mL) and TsOH (109 mg, 0.63 mmol, 3.0 equiv) at room temperature. The resulting mixture was stirred for additional 3 h at room temperature. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in (2R,3R)—N-[(1S)-1-cyano-2-[4-(3-methyl-2-oxo-1,3-benzoxazol-5-yl)phenyl]ethyl]-2-methylpiperidine-3-carboxamide (17.3 mg, 19.49%) as a white solid.

### Analytical Data

LCMS (ES, m/z): [M+H]⁺: 419.2.

¹H NMR (400 MHz, DMSO-d₆) δ 9.32-9.20 (m, 1H), 7.67 (d, J=8.3 Hz, 2H), 7.59 (d, J=1.6 Hz, 1H), 7.46-7.36 (m, 4H), 5.04 (q, J=7.5 Hz, 1H), 3.40 (s, 3H), 3.26-3.11 (m, 2H), 2.91-2.82 (m, 1H), 2.75-2.68 (m, 1H), 2.44 (d, J=10.1 Hz, 1H), 2.31 (s, 1H), 2.23 (d, J=4.2 Hz, 1H), 1.74-1.65 (m, 1H), 1.62-1.52 (m, 1H), 1.43-1.36 (m, 1H), 1.29-1.22 (m, 1H), 0.89 (d, J=6.7 Hz, 3H).

### Example 44. Procedures Used in Synthetic Examples

### Procedure A (TBAF Deprotection)

To a solution of O-TBDMS protected alcohol (1 eq.) in anhydrous THF (6.0 mL/mmol of protected alcohol) was added a 1M TBAF solution in THF (1.5 eq.) at 0° C. under argon atmosphere. The resulting mixture was allowed to warm to room temperature and stirred for 2 h. The reaction mixture was diluted with EtOAc (50 mL) and washed with brine (50 mL), dried over Na₂SO₄ and concentrated under reduced pressure. The crude was purified by flash chromatography over SiO₂ (see conditions for each compound) to afford the expected compound.

### Procedure B (Peptide Coupling)

To a solution of amine derivative (1 eq.) in anhydrous DMF (7.14 mL/mmol of amine) and carboxylic acid derivative (1.05 eq.) were added DIPEA (2.5 eq.) and TBTU (1.5 eq.) at room temperature under argon atmosphere. The reaction mixture was stirred at room temperature for 18 h. The reaction mixture was diluted with EtOAc (10 mL) and water (10 mL). The aqueous layer was extracted with EtOAc (2×10 mL) and the combined organic layers were washed with brine (3×10 mL), dried over Na₂SO₄, filtered and concentrated under reduced pressure. The crude was purified by flash chromatography over SiO₂ (see conditions for each compound) to afford the expected compound.

### Procedure C (N—Boc Deprotection)

To a preheated vial (50° C.) containing Boc protected amine derivative (1 eq.) was added formic acid (7.6 mL/mmol) also preheated at 50° C. The reaction mixture was stirred at 50° C. for 15 min. The reaction mixture was cooled back to room temperature and added dropwise into a cooled (0° C.) mixture of stirred aqueous solution of saturated NaHCO₃ (40 mL) and DCM (40 mL). The layers were separated, and the aqueous layer was extracted with DCM (2×40 mL). The combined organic layers were dried over Na₂SO₄, filtered and concentrated under reduced pressure. The crude was purified by flash chromatography over SiO₂ and/or preparative HPLC (see conditions for each compound) to afford the expected compound.

### Procedure D (O-Bn Deprotection)

To an argon-purged solution of protected alcohol (1 eq.) in EtOH (4.78 mL/mmol of protected alcohol) was added 10% Pd/C (0.1 eq.) at room temperature. The resulting mixture was purged with argon (×3) and then with H₂ (3×). The reaction mixture was stirred under an atmospheric pressure of H₂ at room temperature for 18 h. The reaction mixture was purged with argon, filtered on a pad of celite and rinsed with EtOH (3×5 mL). The filtrate was concentrated under reduced pressure to afford the expected compound.

Procedure E (Alcohol Oxidation into Carboxylic Acid)

To a solution of alcohol derivative (1 eq.) in acetone (16.7 mL/mmol of alcohol) and sodium bromide (0.3 eq.) was added a saturated aqueous solution of NaHCO₃ (2.59 mL/mmol of alcohol) at room temperature. To the resulting mixture were added trichlorocyanuric acid (2.2 eq.) and 2,2,6,6-tetramethylpiperidine-1-oxyl (0.03 eq.) at 0° C. The reaction mixture was allowed to warm to room temperature and stirred for 18 h. Isopropanol (10 mL) was added at room temperature and the reaction mixture was stirred for 30 min. The reaction mixture was diluted with EtOAc (50 mL) and a saturated aqueous solution of NaHCO₃ (50 mL) was added. The two layers were separated, and the aqueous layer was washed with EtOAc (50 mL). The aqueous layer was then acidified with an aqueous solution of 3M HCl until pH ˜1 and extracted with DCM (2×50 mL). The combined organic layers were dried over Na₂SO₄, filtered and concentrated under reduced pressure to afford the expected compound as a crude used as such without further purification.

### Procedure F (O-Alkylation)

To a solution of alcohol derivative (1 eq.) in anhydrous DMF (5.49 mL/mmol of alcohol derivative) and halogenoalkyl (2 eq.) was added NaH 60% in oil (1.1 eq.) at 0° C. under argon atmosphere. The resulting mixture was allowed to warm to room temperature and stirred for 22 h. The reaction mixture was quenched with a saturated aqueous solution of NH₄Cl (10 mL) at room temperature. EtOAc (50 mL) and water (50 mL) were then added and the two layers were separated. The aqueous layer was extracted with EtOAc (2×50 mL) and the combined organic layers were dried over Na₂SO₄, filtered and concentrated under reduced pressure. The crude residue was purified by silica gel flash chromatography (see conditions for each compound) to afford the expected compound.

### Procedure G (N—Boc Protection)

To a solution of amine derivative (1 eq.) in DCM (4 mL/mmol of amine derivative) were added Boc₂O (1.2 eq.) and Et₃N (2 eq.) at room temperature. The resulting mixture was stirred at room temperature for 16 h. The reaction mixture was diluted with water (40 mL) and extracted with DCM (2×60 mL). The combined organic layers were washed with brine (30 mL), dried over Na₂SO₄, filtered and concentrated to dryness under reduced pressure. The crude residue was purified by silica gel flash chromatography (see conditions for each compound) to afford the expected compound.

### Procedure H (Olefin Metathesis)

To a solution of diene (1 eq.) in DCM (63 mL/mmol) was added Benzylidene-bis (tricyclohexylphosphine)dichlororuthenium (0.1 eq.) at room temperature. The resulting mixture was stirred at 55° C. for 8 h. The reaction was concentrated to dryness under reduced pressure. The crude residue was purified by silica gel flash chromatography (see conditions for each compound) to afford the expected compound.

### Procedure I (Hydroboration)

To an argon-purged solution of alkene derivative (1 eq.) in THF (2.5 mL/mmol) was added borane tetrahydrofuran complex solution 1M in THF (1 eq.) at 0° C. The resulting mixture was stirred at 0° C. for 2.5 h. Then, NaOH 3M in water (1 eq.) and H₂O₂ 33% in water (1 eq.) were added sequentially and the resulting mixture was stirred at 0° C. for 3.5 h. The reaction mixture was diluted with water (100 mL) and extracted with EtOAc (3×100 mL). The combined organic layers were washed with brine (100 mL), dried over Na₂SO₄, filtered and concentrated to dryness under reduced pressure. The crude residue was purified by silica gel flash chromatography (see conditions for each compound) to afford the expected compound.

### Example 45. Synthesis of Compound 308A and Compound 308B: (2R)—N-[(1S)-1-cyano-2-[4-(3-methyl-2-oxo-1,3-benzoxazol-5-yl)phenyl]ethyl]-2-methylmorpholine-2-carboxamide and (2S)—N-[(1S)-1-cyano-2-[4-(3-methyl-2-oxo-1,3-benzoxazol-5-yl)phenyl]ethyl]-2-methylmorpholine-2-carboxamide

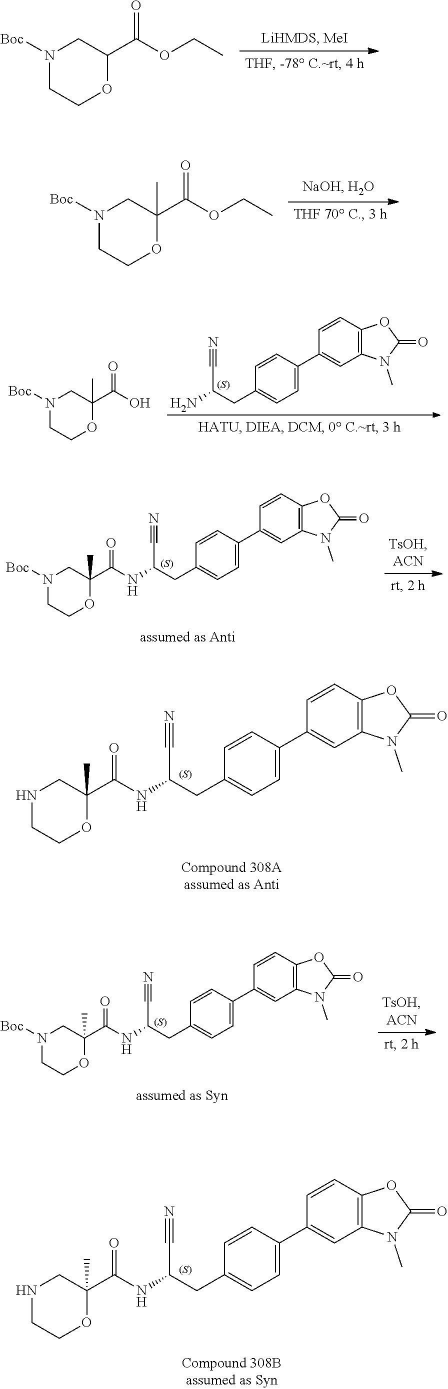

### Synthesis of 4-tert-butyl 2-ethyl 2-methylmorpholine-2,4-dicarboxylate

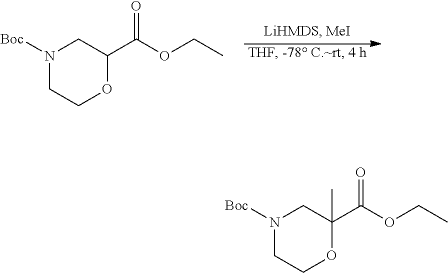

To a stirred solution of 4-tert-butyl 2-ethyl morpholine-2,4-dicarboxylate (500 mg, 1.93 mmol, 1 equiv) and THF (10 mL) was added LiHMDS (5.78 mL, 5.78 mmol, 3 equiv) dropwise/in portions at −78° C. under nitrogen atmosphere. The resulting mixture was stirred for 1 h at −78° C. under nitrogen atmosphere. To the above mixture was added Mel (0.36 mL, 5.78 mmol, 3 equiv) dropwise at −78° C. The resulting mixture was stirred for additional 3 h at room temperature. The reaction was quenched with sat. NH₄Cl (aq.) at room temperature. The resulting mixture was extracted with EtOAc (3×50 mL). The combined organic layers were dried over anhydrous Na₂SO₄. After filtration, the filtrate was concentrated under reduced pressure. The residue was purified by silica gel column chromatography, eluted with PE/EA (5:1) to afford 4-tert-butyl 2-ethyl 2-methylmorpholine-2,4-dicarboxylate (400 mg, 75.89%) as a light-yellow oil. LCMS (ES, m/z): [M+H]⁺: 274.

### Synthesis of 4-(tert-butoxycarbonyl)-2-methylmorpholine-2-carboxylic acid

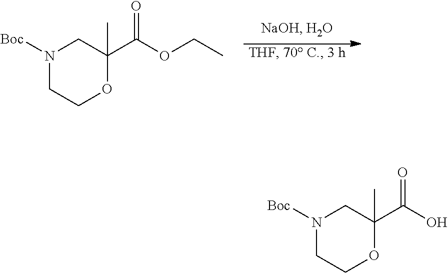

To a stirred solution of 4-tert-butyl 2-ethyl 2-methylmorpholine-2,4-dicarboxylate (400 mg, 1.46 mmol, 1 equiv) and EtOH (5 mL) were added NaOH (176 mg, 4.39 mmol, 3 equiv) in H₂O (5 mL) dropwise at room temperature. The resulting mixture was stirred for 2 h at 70° C. The resulting mixture was concentrated under reduced pressure. The mixture was acidified to pH 4 with conc. HCl. The resulting mixture was extracted with EtOAc (3×50 mL). The combined organic layers were washed with brine (3×30 mL), dried over anhydrous Na₂SO₄. After filtration, the filtrate was concentrated under reduced pressure. This resulted in 4-(tert-butoxycarbonyl)-2-methylmorpholine-2-carboxylic acid (300 mg, 83.58%) as a light-yellow oil. LCMS (ES, m/z): [M+H]⁺: 246.

### Synthesis of tert-butyl 2-{[(1S)-1-cyano-2-[4-(3-methyl-2-oxo-1,3-benzoxazol-5-yl)phenyl]ethyl]carbamoyl}-2-methylmorpholine-4-carboxylate

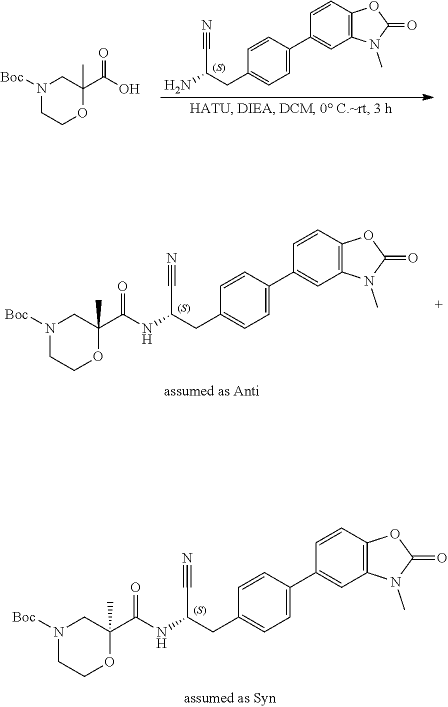

To a stirred mixture of 4-(tert-butoxycarbonyl)-2-methylmorpholine-2-carboxylic acid (163 mg, 0.66 mmol, 1.5 equiv), (2S)-2-amino-3-[4-(3-methyl-2-oxo-1,3-benzoxazol-5-yl)phenyl]propanenitrile (130 mg, 0.44 mmol, 1.00 equiv) and DIEA (172 mg, 1.33 mmol, 3 equiv) in DCM (5 mL) were added HATU (202 mg, 0.53 mmol, 1.2 equiv) in portions at 0° C. The resulting mixture was stirred for additional 3 h at 0° C. The residue was purified by silica gel column chromatography, eluted with PE/THF (1:1) to afford tert-butyl 2-{[(1S)-1-cyano-2-[4-(3-methyl-2-oxo-1,3-benzoxazol-5-yl)phenyl]ethyl]carbamoyl}-2-methylmorpholine-4-carboxylate (100 mg, 86.69%) and tert-butyl 2-{[(1S)-1-cyano-2-[4-(3-methyl-2-oxo-1,3-benzoxazol-5-yl)phenyl]ethyl]carbamoyl}-2-methylmorpholine-4-carboxylate (100 mg, 86.69%) as a light yellow oil. LCMS (ES, m/z): [M+H]⁺: 521.

### Synthesis of (2R)—N-[(1S)-1-cyano-2-[4-(3-methyl-2-oxo-1,3-benzoxazol-5-yl)phenyl]ethyl]-2-methylmorpholine-2-carboxamide

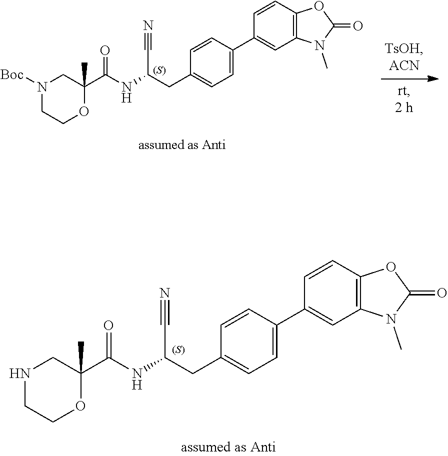

Into a 50 mL round-bottom flask were added tert-butyl (2R)-2-{[(1S)-1-cyano-2-[4-(3-methyl-2-oxo-1,3-benzoxazol-5-yl)phenyl]ethyl]carbamoyl}-2-methylmorpholine-4-carboxylate (110 mg, 0.21 mmol, 1 equiv), TsOH·H₂O (121 mg, 0.63 mmol, 3 equiv) and ACN (3 mL) at room temperature. The resulting mixture was stirred for additional 2 h at room temperature. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in (2R)—N-[(1S)-1-cyano-2-[4-(3-methyl-2-oxo-1,3-benzoxazol-5-yl)phenyl]ethyl]-2-methylmorpholine-2-carboxamide (21.2 mg, 23.86%) as a white solid.

### Analytical Data

LCMS (ES, m/z): [M+H]⁺: 421.

¹H NMR (400 MHz, DMSO-d₆) δ 8.71 (d, J=8.4 Hz, 1H), 7.70-7.62 (m, 2H), 7.58 (t, J=1.2 Hz, 1H), 7.45-7.36 (m, 4H), 5.04 (p, J=8.1, 7.5 Hz, 1H), 3.55-3.47 (m, 1H), 3.41 (s, 3H), 3.30-3.18 (m, 3H), 3.02 (d, J=12.4 Hz, 1H), 2.60-2.42 (m, 2H), 2.35 (d, J=12.4 Hz, 1H), 1.96 (br, 1H), 1.18 (s, 3H).

### Synthesis of (2S)—N-[(1S)-1-cyano-2-{4-[1-(1-methylpiperidin-4-yl) indazol-6-yl]phenyl}ethyl]-1,4-oxazepane-2-carboxamide

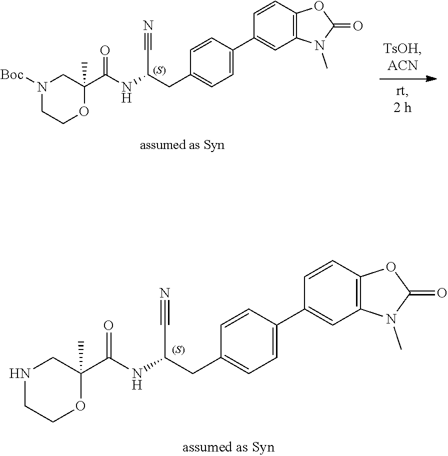

Into a 50 mL round-bottom flask were added tert-butyl (2S)-2-{[(1S)-1-cyano-2-[4-(3-methyl-2-oxo-1,3-benzoxazol-5-yl)phenyl]ethyl]carbamoyl}-2-methylmorpholine-4-carboxylate (100 mg, 0.19 mmol, 1 equiv), TsOH (99 mg, 0.58 mmol, 3 equiv) and ACN (3 mL) at room temperature. The resulting mixture was stirred for additional 2 h at room temperature. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in (2S)—N-[(1S)-1-cyano-2-[4-(3-methyl-2-oxo-1,3-benzoxazol-5-yl)phenyl]ethyl]-2-methylmorpholine-2-carboxamide (21.2 mg, 26.25%) as a white solid.

### Analytical Data

LCMS (ES, m/z): [M+H]⁺: 421.

¹H NMR (400 MHz, DMSO-d₆) δ 8.71 (d, J=8.6 Hz, 1H), 7.70-7.62 (m, 2H), 7.57 (t, J=1.2 Hz, 1H), 7.45-7.35 (m, 4H), 5.14-5.00 (m, 1H), 3.65-3.57 (m, 1H), 3.44 (td, J=7.6, 4.0 Hz, 1H), 3.41 (s, 3H), 3.28-3.16 (m, 2H), 3.09 (d, J=12.4 Hz, 1H), 2.64-2.56 (m, 2H), 2.37 (d, J=12.3 Hz, 1H), 2.15 (br, 1H), 0.97 (s, 3H).

### Example 46. Synthesis of Compound 257

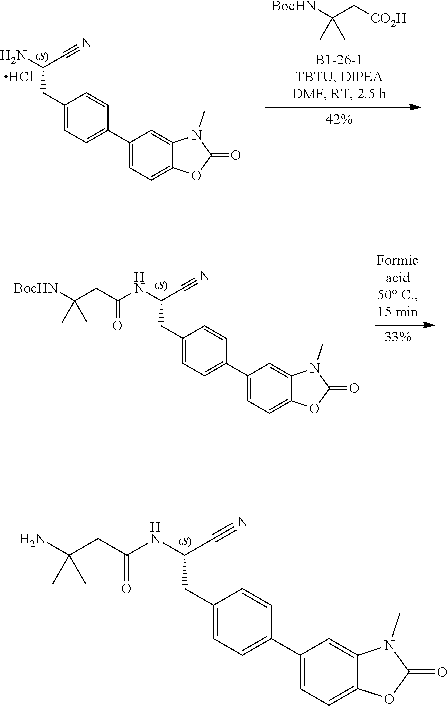

### tert-butyl N-(1-{[(1S)-1-cyano-2-[4-(3-methyl-2-oxo-2,3-dihydro-1,3-benzoxazol-5-yl)phenyl]ethyl]carbamoyl}-2-methylpropan-2-yl) carbamate

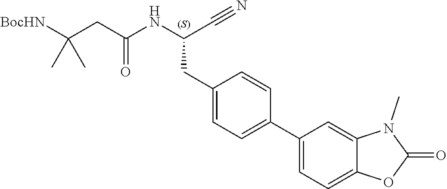

Starting from (S)-2-amino-3-(4-(3-methyl-2-oxo-2,3-dihydrobenzo[d]oxazol-5-yl)phenyl)propanenitrile (1 eq., 310 mg, 0.94 mmol) and 3-((tert-butoxycarbonyl)amino)-3-methylbutanoic acid (1.2 eq., 245.08 mg, 1.13 mmol) and using general procedure B, tert-butyl N-(1-{[(1S)-1-cyano-2-[4-(3-methyl-2-oxo-2,3-dihydro-1,3-benzoxazol-5-yl)phenyl]ethyl]carbamoyl}-2-methylpropan-2-yl) carbamate was obtained as a white solid (193 mg, 42%) after purification by flash chromatography over silica gel (50 μm, 24 g, cyclohexane/EtOAc from 100:0 to 20:80 in 35 min).

LC/MS (AN01_001_012): Rt=2.49 min, 100%, [M−Boc+H]⁺=393.3.

¹H NMR (400 MHz, CDCl₃) δ 7.48 (d, J=7.9 Hz, 2H), 7.29 (d, J=7.9 Hz, 2H), 7.24 (d, J=8.3 Hz, 1H), 7.19 (d, J=8.3 Hz, 1H), 7.06 (s, 1H), 6.47 (d, J=8.7 Hz, 1H), 5.08 (q, J=7.0 Hz, 1H), 4.62 (s, 1H), 3.39 (s, 3H), 3.02 (ddd, J=13.8, 10.5, 6.1 Hz, 2H), 2.82 (d, J=13.1 Hz, 1H), 2.43 (d, J=13.1 Hz, 1H), 1.34 (s, 9H), 1.27 (d, J=4.1 Hz, 6H).

### 3-amino-N-[(1S)-1-cyano-2-[4-(3-methyl-2-oxo-2,3-dihydro-1,3-benzoxazol-5-yl)phenyl]ethyl]-3-methylbutanamide Compound 257

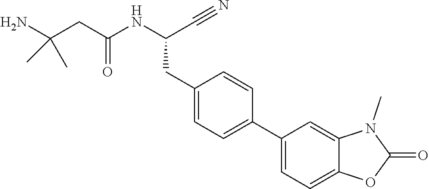 [SMILES: Cn1c(=O)oc2ccc(-c3ccc(C[C@@H](C#N)NC(=O)CC(C)(C)N)cc3)cc21]

Starting from tert-butyl N-(1-{[(1S)-1-cyano-2-[4-(3-methyl-2-oxo-2,3-dihydro-1,3-benzoxazol-5-yl)phenyl]ethyl]carbamoyl}-2-methylpropan-2-yl) carbamate (1 eq., 138 mg, 0.28 mmol) and using general procedure C, 3-amino-N-[(1S)-1-cyano-2-[4-(3-methyl-2-oxo-2,3-dihydro-1,3-benzoxazol-5-yl)phenyl]ethyl]-3-methylbutanamide was obtained as a white solid (36.3 mg, 33%) after purification by flash chromatography over silica gel (12 g, gradient: DCM/MeOH from 100:0 to 70:30 in 35 min).

LC/MS (AN_01_001_026_DEDL): Rt=7.39 min, 98.2%, [M+H]⁺=393.5.

¹H NMR (400 MHz, DMSO) δ 9.11 (s, 1H), 7.72-7.61 (m, 2H), 7.61-7.55 (m, 1H), 7.47-7.35 (m, 4H), 5.03 (t, J=7.7 Hz, 1H), 3.41 (s, 3H), 3.14 (ddd, J=13.7, 12.0, 7.4 Hz, 2H), 2.28-1.86 (m, 4H), 1.00 (s, 6H).

### Example 47. Synthesis of Compound 238

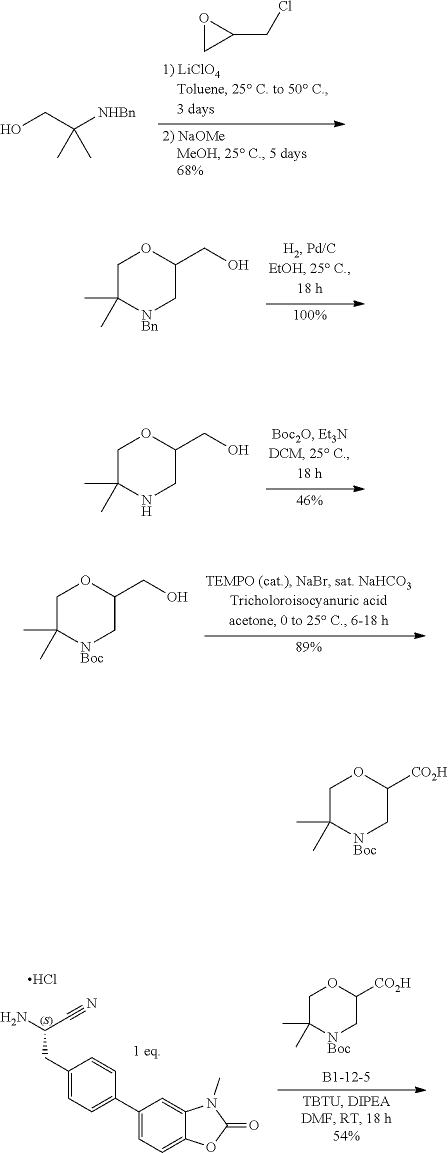

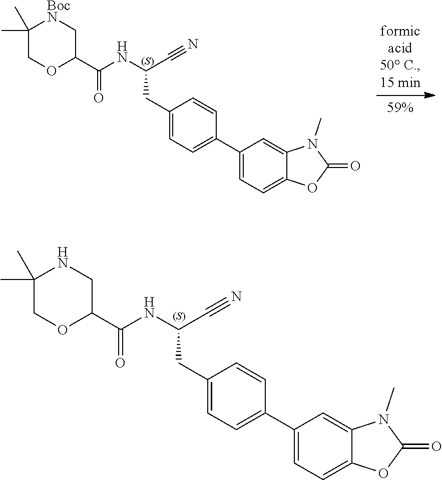

### (4-benzyl-5,5-dimethylmorpholin-2-yl)methanol

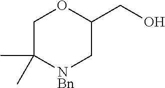 [SMILES: CC1(C)COC(CO)CN1Cc1ccccc1]

To a solution of 2-(benzylamino)-2-methylpropan-1-ol (1 eq., 270 mg, 1.506 mmol) in toluene (7.53 mL) was added epichlorohydrin (1.3 eq., 181.16 mg, 0.15 mL, 1.96 mmol) and LiClO₄ (1.3 eq., 208.31 mg, 1.96 mmol) at room temperature. The reaction mixture was stirred at room temperature for 18 h, then stirred at 50° C. for 6 h. LiClO₄ (1.3 eq., 208.31 mg, 1.96 mmol) and epichlorohydrin (0.5 eq., 69.68 mg, 0.059 mL, 0.75 mmol) were added and the reaction mixture was stirred for 24 h at 50° C. LiClO₄ (1.3 eq., 208.31 mg, 1.96 mmol) and epichlorohydrin (0.5 eq., 69.68 mg, 0.059 mL, 0.75 mmol) were added and the reaction mixture was stirred at 50° C. for 24 h. The reaction mixture was cold to room temperature. NaOMe (2.5 eq., 203.42 mg, 3.77 mmol) and MeOH (2 mL) were added at room temperature and the reaction mixture was stirred at room temperature for 24 h. NaOMe (2.5 eq., 203.42 mg, 3.77 mmol) was added and the reaction mixture was stirred at room temperature for 3 days. The reaction mixture was quenched with NH₄Cl (50 mL) and extracted with EtOAc (3×50 mL). The combined organic layers were washed with brine (50 mL), dried over Na₂SO₄, filtered and concentrated to dryness under reduced pressure. The residue was dissolved in MeOH (10 mL). NaOMe (2.5 eq., 203.42 mg, 3.77 mmol) was added and the reaction mixture was stirred at room temperature for 24 h. NH₄Cl (50 mL) was added and the aqueous layer was extracted with EtOAc (3×50 mL). The combined organic layer was washed with brine (50 mL), dried over Na₂SO₄, filtered and concentrated under reduced pressure. The crude was purified by flash chromatography over silica gel (50 μm, 12 g, Cyclohexane/EtOAc from 100:0 to 50:50 over 35 min) to afford (4-benzyl-5,5-dimethylmorpholin-2-yl)methanol as a colorless oil (242 mg, 68%).

LC/MS (AN01_001_012): Rt=0.60-0.84 min, ND, [M+H]⁺=236.2.

¹H NMR (400 MHz, DMSO) δ 7.39-7.26 (m, 4H), 7.26-7.16 (m, 1H), 4.55 (dd, J=6.1, 5.3 Hz, 1H), 3.99 (d, J=13.8 Hz, 1H), 3.42 (d, J=10.8 Hz, 1H), 3.39-3.29 (m, 2H), 3.28-3.16 (m, 2H), 2.94 (d, J=13.9 Hz, 1H), 2.37 (dd, J=11.8, 2.5 Hz, 1H), 2.09 (dd, J=11.9, 10.2 Hz, 1H), 1.14-0.94 (m, 6H).

### (5,5-dimethylmorpholin-2-yl)methanol

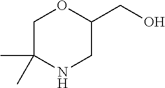

Starting from (4-benzyl-5,5-dimethylmorpholin-2-yl)methanol (1 eq., 200 mg, 0.85 mmol) and using the general procedure D, (5,5-dimethylmorpholin-2-yl)methanol was obtained as a colorless oil (123 mg, 100%).

LC/MS (AN01_001_012): Rt=0.28 min, ND, [M+H]⁺=146.0.

¹H NMR (400 MHz, DMSO) δ 4.57 (s, 1H), 3.49-3.33 (m, 3H), 3.31-3.24 (m, 1H), 3.20 (dq, J=7.2, 5.7 Hz, 1H), 3.08 (d, J=10.7 Hz, 1H), 2.63-2.55 (m, 2H), 1.07 (s, 3H), 0.88 (s, 3H).

### tert-butyl 2-(hydroxymethyl)-5,5-dimethylmorpholine-4-carboxylate

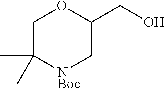 [SMILES: CC(C)(C)OC(=O)N1CC(CO)OCC1(C)C]

To a solution of (5,5-dimethylmorpholin-2-yl)methanol (1 eq., 125 mg, 0.86 mmol) and Et₃N (1.01 eq., 87.99 mg, 0.12 mL, 0.87 mmol) in DCM (2.58 mL) was added a solution of Boc₂O (1.2 eq., 225.46 mg, 0.22 mL, 1.033 mmol). The reaction mixture was stirred at room temperature for 18 h. The reaction mixture was concentrated under reduced pressure. The crude was purified by flash chromatography over silica gel (50 μm, 12 g, Cyclohexane/EtOAc from 100:0 to 70:30 over 50 min) to afford tert-butyl 2-(hydroxymethyl)-5,5-dimethylmorpholine-4-carboxylate as a colorless oil (98 mg, 46%).

LC/MS (AN01_001_012): Rt=2.12 min, ND, [M−Boc+H]⁺=146.1.

¹H NMR (400 MHz, DMSO) δ 4.76 (dd, J=6.0, 5.1 Hz, 1H), 3.67 (dd, J=13.5, 3.1 Hz, 1H), 3.51-3.39 (m, 3H), 3.39-3.32 (m, 1H), 3.22 (d, J=11.8 Hz, 1H), 2.87-2.76 (m, 1H), 1.40 (s, 9H), 1.31 (s, 3H), 1.19 (s, 3H).

### 4-[(tert-butoxy)carbonyl]-5,5-dimethylmorpholine-2-carboxylic acid

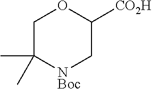 [SMILES: CC(C)(C)OC(=O)N1CC(C(=O)O)OCC1(C)C]

Starting from tert-butyl 2-(hydroxymethyl)-5,5-dimethylmorpholine-4-carboxylate (1 eq., 90 mg, 0.37 mmol) and using the general procedure E, 4-[(tert-butoxy)carbonyl]-5,5-dimethylmorpholine-2-carboxylic acid was obtained as a white solid (84.5 mg, 89%).

LC/MS (AN01_001_012): Rt=2.12 min, ND, [M−Boc+H]⁺=160.0.

¹H NMR (400 MHz, DMSO) δ 12.92 (s, 1H), 4.20 (dd, J=9.4, 4.3 Hz, 1H), 3.81 (dd, J=13.8, 4.3 Hz, 1H), 3.53 (d, J=11.8 Hz, 1H), 3.32 (d, J=11.8 Hz, 1H), 3.24 (dd, J=13.8, 9.4 Hz, 1H), 1.40 (s, 9H), 1.31 (s, 3H), 1.23 (s, 3H).

### tert-butyl 2-{[(1S)-1-cyano-2-[4-(3-methyl-2-oxo-2,3-dihydro-1,3-benzoxazol-5-yl)phenyl]ethyl]carbamoyl}-5,5-dimethylmorpholine-4-carboxylate

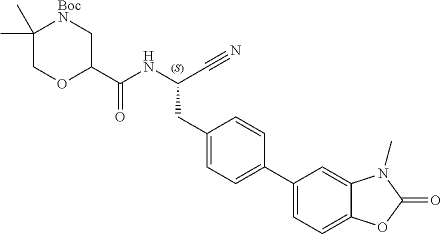

Starting from (S)-2-amino-3-(4-(3-methyl-2-oxo-2,3-dihydrobenzo[d]oxazol-5-yl)phenyl)propanenitrile hydrochloride (1 eq., 96.9 mg, 0.29 mmol) and 4-(tert-butoxycarbonyl)-5,5-dimethylmorpholine-2-carboxylic acid (1.05 eq., 80 mg, 0.309 mmol) and using general procedure B, tert-butyl 2-{[(1S)-1-cyano-2-[4-(3-methyl-2-oxo-2,3-dihydro-1,3-benzoxazol-5-yl)phenyl]ethyl]carbamoyl}-5,5-dimethylmorpholine-4-carboxylate was obtained as an orange solid (87.9 mg, 56%) after purification by flash chromatography over silica gel (50 μm, 12 g, cyclohexane/EtOAc from 100:0 to 60:40 over 45 min).

LC/MS (AN01_001_012): Rt=2.59 min, 100%, [M−Boc+H]⁺=435.1.

¹H NMR (400 MHz, DMSO) δ 8.85 (dd, J=8.4, 3.0 Hz, 1H), 7.70-7.62 (m, 2H), 7.62-7.53 (m, 1H), 7.45-7.34 (m, 4H), 5.14-4.97 (m, 1H), 4.11 (ddd, J=11.9, 10.1, 4.2 Hz, 1H), 3.74 (ddd, J=65.2, 13.8, 4.2 Hz, 1H), 3.55 (dd, J=28.9, 11.8 Hz, 1H), 3.40 (s, 3H), 3.38-3.32 (m, 1H), 3.28-3.15 (m, 2H), 2.91 (ddd, J=152.8, 13.8, 10.1 Hz, 1H), 1.43-1.28 (m, 12H), 1.20 (d, J=20.3 Hz, 3H).

### N-[(1S)-1-cyano-2-[4-(3-methyl-2-oxo-2,3-dihydro-1,3-benzoxazol-5-yl)phenyl]ethyl]-5,5-dimethylmorpholine-2-carboxamide

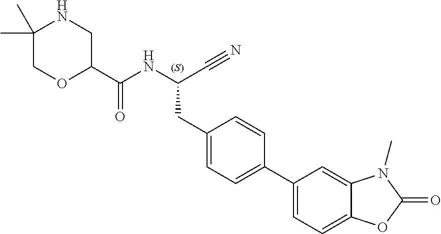

Starting from tert-butyl 2-{[(1S)-1-cyano-2-[4-(3-methyl-2-oxo-2,3-dihydro-1,3-benzoxazol-5-yl)phenyl]ethyl]carbamoyl}-5,5-dimethylmorpholine-4-carboxylate (1 eq., 75 mg, 0.14 mmol) and using general procedure C, N-[(1S)-1-cyano-2-[4-(3-methyl-2-oxo-2,3-dihydro-1,3-benzoxazol-5-yl)phenyl]ethyl]-5,5-dimethylmorpholine-2-carboxamide was obtained as a white solid (35.8 mg, 59%) after purification by flash chromatography over silica gel (15 μm, 4 g, DCM/MeOH from 100:0 to 90:10 over 60 min).

LC/MS (AN_01_001_021_DEDL): Rt=6.70 min, 98.7%, [M+H]⁺=435.5.

¹H NMR (400 MHz, DMSO) δ 8.68 (dd, J=8.5, 2.1 Hz, 1H), 7.76-7.65 (m, 2H), 7.65-7.59 (m, 1H), 7.51-7.34 (m, 4H), 5.16-4.99 (m, 1H), 3.78 (td, J=10.2, 3.3 Hz, 1H), 3.58 (dd, J=10.8, 9.5 Hz, 1H), 3.46 (s, 3H), 3.25 (ddd, J=15.7, 9.6, 4.2 Hz, 3H), 2.91-2.73 (m, 1H), 2.72-2.43 (m, 1H), 2.18 (s, 1H), 1.14 (d, J=4.9 Hz, 3H), 0.96 (d, J=5.7 Hz, 3H).

### Example 50. Synthesis of Compound 239: N—((S)-1-cyano-2-(4-(3-methyl-2-oxo-2,3-dihydrobenzo[d]oxazol-5-yl)phenyl)ethyl)-2-oxa-6-azaspiro[3.4]octane-8-carboxamide

### Ethyl 6-benzyl-2-oxa-6-azaspiro[3.4]octane-8-carboxylate

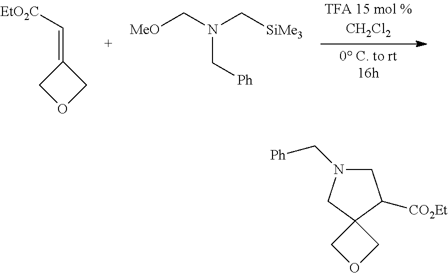 [SMILES: CCOC(=O)C=C1COC1 ; COCN(Cc1ccccc1)C[Si](C)(C)C ; CCOC(=O)C1CN(Cc2ccccc2)CC12COC2]

Ethyl 2-(oxetan-3-ylidene)acetate (2.000 g, 1 Eq, 14.07 mmol) was dissolved in in EtOAc (20.01 mL). The solution was cooled to 0° C. N-(Methoxymethyl)-N-[(trimethylsilyl)methyl]benzylamine (4.454 g, 4.80 mL, 90% Wt, 1.2 Eq, 16.88 mmol) was added slowly to the solution before the dropwise addition of a solution of trifluoroacetic acid (240.6 mg, 162.6 μL, 0.15 Eq, 2.110 mmol) in EtOAc (1.668 mL). The reaction mixture was allowed to warm to rt overnight. A saturated NaHCO₃ aqueous solution was slowly added and the layers were separated. The aqueous layer was extracted three times with EtOAc and the combined organic layers were dried over anhydrous Na₂SO₄, filtered and concentrated to dryness. The crude product was used as it in the next step without further purification.

¹H NMR (400 MHz, CDCl₃) δ 7.31 (m, 5H), 4.72-4.63 (m, 2H), 4.51-4.42 (m, 2H), 4.21-4.17 (m, 2H), 3.64-3.63 (m, 2H), 3.07 (m, 2H), 2.75 (m, 1H), 2.62 (m, 1H), 2.43-2.37 (m, 1H), 1.31-1.28 (m, 3H).

### Ethyl 2-oxa-6-azaspiro[3.4]octane-8-carboxylate

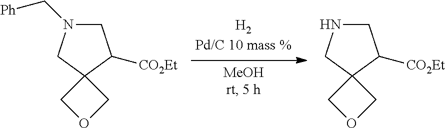 [SMILES: CCOC(=O)C1CN(Cc2ccccc2)CC12COC2 ; CCOC(=O)C1CNCC12COC2]

A flask was charged with ethyl 6-benzyl-2-oxa-6-azaspiro[3.4]octane-8-carboxylate (3.874 g, 1 Eq, 14.07 mmol), palladium (862.5 mg, 0.576 Eq, 8.104 mmol) and MeOH (70.35 mL). Evacuated and backfilled with N₂ 3 times then with H₂ three times. After 5 hours, the reaction mixture was filtered and the filtrate was concentrated too dryness.

¹H NMR (400 MHz, CDCl₃) δ 4.71 (dd, J=6.3, 3.3 Hz, 2H), 4.51 (dd, J=6.4, 2.4 Hz, 2H), 4.19 (qd, J=7.1, 4.1 Hz, 2H), 3.18-3.09 (m, 2H), 2.72 (t, J=7.6 Hz, 2H), 2.47 (d, J=9.6 Hz, 1H), 1.30 (t, J=7.1 Hz, 3H).

### 6-benzyl 8-ethyl 2-oxa-6-azaspiro[3.4]octane-6,8-dicarboxylate

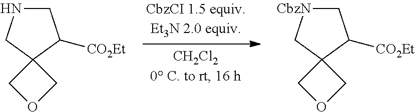 [SMILES: CCOC(=O)C1CNCC12COC2 ; CCOC(=O)C1CN(C(=O)OCc2ccccc2)CC12COC2]

Ethyl 2-oxa-6-azaspiro[3.4]octane-8-carboxylate (1.303 g, 1 Eq, 7.035 mmol) was dissolved in DCM (35.17 mL). The solution was cooled to 0° C. before slow addition of Triethylamine (1.424 g, 1.96 mL, 2.0 Eq, 14.07 mmol) and Carbobenzoxy chloride (1.800 g, 1.596 mL, 1.5 Eq, 10.55 mmol). The reaction mixture was allowed to warm to rt overnight. Addition of sat NaHCO₃, extraction with DCM. Combined organic layers were dried over Na₂SO₄, filtered and concentrated to dryness. Purif by MPLC (cyclohexane:EtOAc) to afford 6-benzyl 8-ethyl 2-oxa-6-azaspiro[3.4]octane-6,8-dicarboxylate (2.25 g, 7.04 mmol, 100%).

¹H NMR (400 MHz, CDCl₃) δ 7.29 (m, 5H), 5.06 (d, J=3.1 Hz, 2H), 4.68 (dd, J=7.0, 1.8 Hz, 1H), 4.61 (dd, J=9.1, 6.1 Hz, 1H), 4.56-4.43 (m, 2H), 4.14 (pd, J=7.2, 3.3 Hz, 2H), 3.74 (d, J=8.1 Hz, 2H), 3.71-3.60 (m, 1H), 3.50 (ddd, J=11.6, 7.3, 4.5 Hz, 1H), 3.15-3.08 (m, 1H), 1.22 (t, J=7.1 Hz, 3H).

### 6-((benzyloxy)carbonyl)-2-oxa-6-azaspiro[3.4]octane-8-carboxylic acid

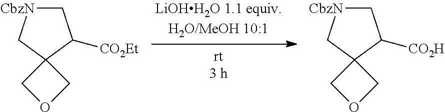 [SMILES: CCOC(=O)C1CN(C(=O)OCc2ccccc2)CC12COC2 ; O=C(O)C1CN(C(=O)OCc2ccccc2)CC12COC2]

6-benzyl 8-ethyl 2-oxa-6-azaspiro[3.4]octane-6,8-dicarboxylate (2.247 g, 1 Eq, 7.035 mmol) was dissolved in a mixture of MeOH (28.14 mL) and H₂O (2.814 mL) before being cooled to 0° C. Aqueous Lithium hydroxide monohydrate (324.7 mg, 2.845 mL, 2.72 molar, 1.1 Eq, 7.739 mmol) was added dropwise and the reaction mixture was stirred at room temperature for 3 hours. The solvent was removed under vacuum. The residue was transferred to a separatory funnel, extracted with EtOAc three times. The aqueous phase was acidified with KHSO₄ 1N. Then extracted with EtOAc three times, dried over Na₂SO₄ and concentrated to dryness to afford crude carboxylic acid (2.0 g, quant.).

### (R)-2-amino-3-(4-(3-methyl-2-oxo-2,3-dihydrobenzo[d]oxazol-5-yl)phenyl)propanenitrile

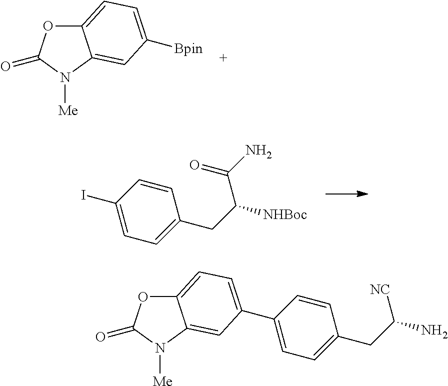

### benzyl 8-(((R)-1-cyano-2-(4-(3-methyl-2-oxo-2,3-dihydrobenzo[d]oxazol-5-yl)phenyl)ethyl) carbamoyl)-2-oxa-6-azaspiro[3.4]octane-6-carboxylate

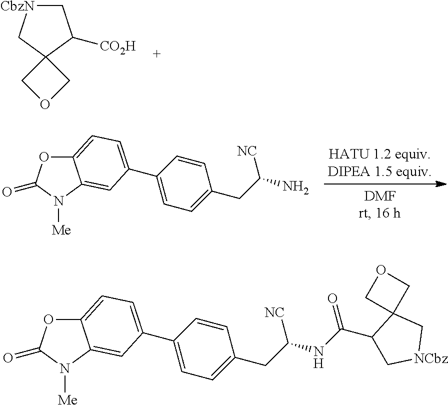 [SMILES: O=C(O)C1CN(C(=O)OCc2ccccc2)CC12COC2 ; Cn1c(=O)oc2ccc(-c3ccc(C[C@@H](N)C#N)cc3)cc21 ; Cn1c(=O)oc2ccc(-c3ccc(C[C@H](C#N)NC(=O)C4CN(C(=O)OCc5ccccc5)CC45COC5)cc3)cc21]

To a solution of (S)-2-amino-3-(4-(3-methyl-2-oxo-2,3-dihydrobenzo[d]oxazol-5-yl)phenyl)propanenitrile (188.9 mg, 1 Eq, 644.1 μmol) and 6-((benzyloxy)carbonyl)-2-oxa-6-azaspiro[3.4]octane-8-carboxylic acid (187.6 mg, 1 Eq, 644.1 μmol) in dry DMF (3.221 mL) was added HATU (293.9 mg, 1.2 Eq, 772.9 μmol) and Diisopropylethylamine (124.9 mg, 167 μL, 1.5 Eq, 966.2 μmol). The reaction mixture was stirred at rt for 16 hour. Addition of NaHCO₃ (30 mL) at 0° C., and extracted with ethyl acetate (10 mL×3). The combined organic layers were washed with brine (20 mL), dried over Na₂SO₄, filtered and concentrated under reduced pressure to give crude product. Crude dissolved in DCM and Et₂O was added->white/beige precipitate (302 mg). LCMS: not pure. Purification by MPLC (DCM: MeOH to 10%) afford still impure product. It was repurified by FC with Cyclohexane to EtOAc 100% to afford benzyl 8-(((S)-1-cyano-2-(4-(3-methyl-2-oxo-2,3-dihydrobenzo[d]oxazol-5-yl)phenyl)ethyl) carbamoyl)-2-oxa-6-azaspiro[3.4]octane-6-carboxylate (166.5 mg, 293.9 μmol, 45.62%)

¹H NMR (400 MHz, CDCl₃) δ 7.53 (d, J=8.2 Hz, 2H), 7.39-7.27 (m, 9H), 7.05 (s, 1H), 6.59 (s, 1H), 5.23 (d, J=8.1 Hz, 1H), 5.11 (d, J=10.1 Hz, 2H), 4.68-4.58 (m, 2H), 4.55 (s, 1H), 4.50 (d, J=4.6 Hz, 2H), 3.89-3.77 (m, 2H), 3.68-3.56 (m, 2H), 3.39 (d, J=1.6 Hz, 3H), 3.16 (dd, J=13.7, 6.5 Hz, 2H).

### N—((R)-1-cyano-2-(4-(3-methyl-2-oxo-2,3-dihydrobenzo[d]oxazol-5-yl)phenyl)ethyl)-2-oxa-6-azaspiro[3.4]octane-8-carboxamide

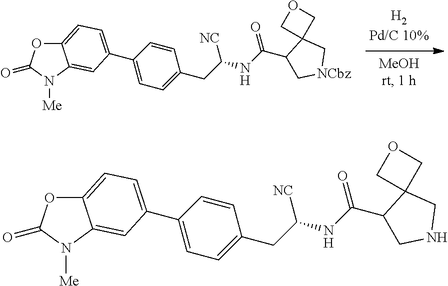 [SMILES: Cn1c(=O)oc2ccc(-c3ccc(C[C@H](C#N)NC(=O)C4CN(C(=O)OCc5ccccc5)CC45COC5)cc3)cc21 ; Cn1c(=O)oc2ccc(-c3ccc(C[C@H](C#N)NC(=O)C4CNCC45COC5)cc3)cc21]

A flask was charged with benzyl 8-(((S)-1-cyano-2-(4-(3-methyl-2-oxo-2,3-dihydrobenzo[d]oxazol-5-yl)phenyl)ethyl) carbamoyl)-2-oxa-6-azaspiro[3.4]octane-6-carboxylate (166.500 mg, 1 Eq, 293.85 μmol), palladium (31.272 mg, 10% Wt, 0.1 Eq, 29.385 μmol) and MeOH (2.9385 mL). Evacuated and backfilled with N₂ 3 times then with H₂ three times. After 1 hour, the reaction mixture was filtered and the filtrate was concentrated too dryness. The residue was purified by preparative analytical HPLC to afford N—((S)-1-cyano-2-(4-(3-methyl-2-oxo-2,3-dihydrobenzo[d]oxazol-5-yl)phenyl)ethyl)-2-oxa-6-azaspiro[3.4]octane-8-carboxamide (13 mg, 11%)

¹H NMR (500 MHz, DMSO-d6) δ 9.04 (dd, J=7.8, 5.5 Hz, 1H), 7.66 (tdd, J=8.5, 4.1, 2.0 Hz, 2H), 7.60-7.54 (m, 1H), 7.45-7.37 (m, 4H), 5.11-4.97 (m, 1H), 4.53-4.46 (m, 1H), 4.33 (d, J=5.8 Hz, 1H), 4.29-4.26 (m, 1H), 4.12-3.96 (m, 2H), 3.72-3.64 (m, 1H), 3.51 (t, J=1.1 Hz, 3H), 3.22-3.10 (m, 5H), 3.05-2.92 (m, 2H), 2.87 (ddd, J=14.7, 9.5, 5.3 Hz, 1H), 2.82-2.74 (m, 1H), 2.65 (s, 1H).

### Example 51. Synthesis of Compound 227: (3S,4S)-(+/−)-cis-3-amino-N-[(1S)-1-cyano-2-[4-(3-methyl-2-oxo-2,3-dihydro-1,3-benzoxazol-5-yl)phenyl]ethyl]piperidine-4-carboxamide

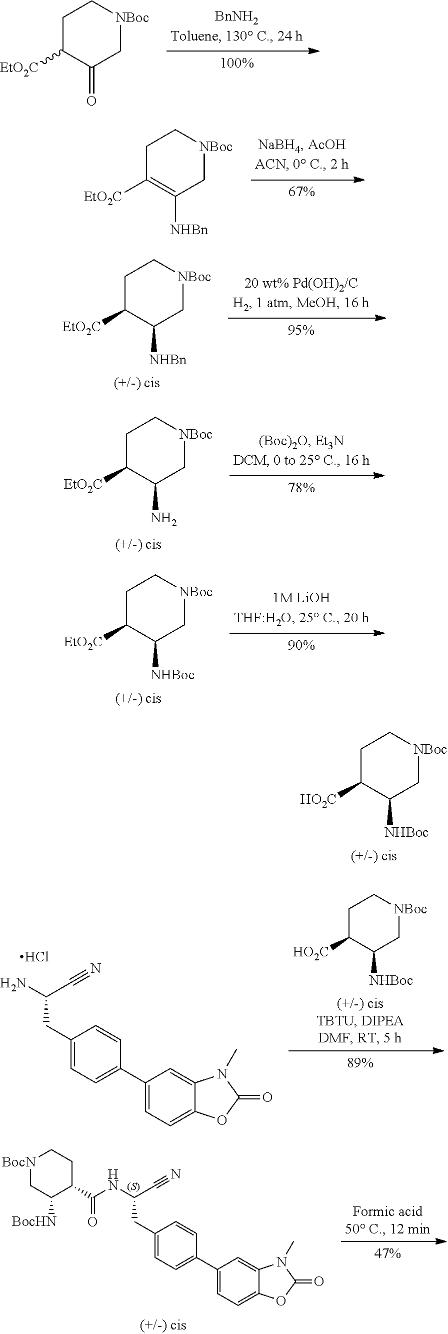

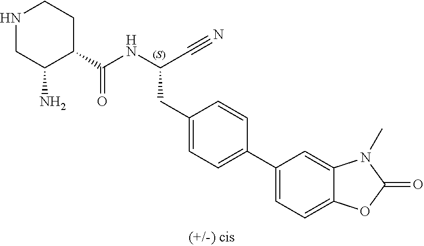

### 1-tert-butyl 4-ethyl 5-(benzylamino)-1,2,3,6-tetrahydropyridine-1,4-dicarboxylate

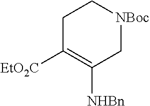

A solution of 1-(tert-butyl) 4-ethyl (3S,4S)-3-(benzylamino) piperidine-1,4-dicarboxylate-1-(tert-butyl) 4-ethyl 3-oxopiperidine-1,4-dicarboxylate-1-(tert-butyl) 4-ethyl 5-(benzylamino)-3,6-dihydropyridine-1,4(2H)-dicarboxylate (1 eq., 5.18 g, 0.019 mol) and benzylamine (1.2 eq., 2.46 g, 2.505 mL, 0.023 mol) in toluene (80 mL) was heated under reflux (130° C.) for 24 h. The solution was cooled to room temperature and then evaporated to dryness to afford 1-tert-butyl 4-ethyl 5-(benzylamino)-1,2,3,6-tetrahydropyridine-1,4-dicarboxylate as a yellow solid (6.9 g, 100%).

LC/MS (AN01_001_012): Rt=2.88 min, 79.6%, [M+H]⁺=361.3.

¹H NMR (400 MHz, CDCl₃) δ 9.04 (s, 1H), 7.31-7.14 (m, 5H), 4.30 (d, J=6.3 Hz, 2H), 4.16-3.99 (m, 4H), 3.34 (t, J=5.9 Hz, 2H), 2.34-2.26 (m, 2H), 1.45-1.26 (m, 9H), 1.20 (t, J=7.1 Hz, 3H).

### 1-tert-butyl 4-ethyl (3S,4S)-(+/−)-cis-3-(benzylamino) piperidine-1,4-dicarboxylate

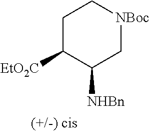

To a solution of 1-tert-butyl 4-ethyl 5-(benzylamino)-1,2,3,6-tetrahydropyridine-1,4-dicarboxylate (1 eq., 7.5 g, 0.019 mol) in acetonitrile (91.701 mL) and acetic acid (10 eq., 11.46 g, 10.93 mL, 0.19 mol) was added at 0° C. sodium tetrahydroborate (1.35 eq., 0.97 g, 0.026 mol). The resulting mixture was warmed to room temperature and was stirred for 2 h. EtOAc (150 mL) was added, and the reaction mixture was slowly quenched with sat. NaHCO₃ solution (50 mL). The two layers were separated and the organic layer was washed with sat. NaHCO₃ solution (2×150 mL). The aqueous layer was extracted with EtOAc (150 mL). The combined organic layers were dried over Na₂SO₄, filtered and concentrated to dryness under reduced pressure. The crude was purified by flash chromatography over silica gel (50 μm, 220 g, dry loading, gradient: cyclohexane/EtOAc from 100/0 to 75/25 over 18 CV) to afford 1-tert-butyl 4-ethyl (3S,4S)-(+/−)-cis-3-(benzylamino) piperidine-1,4-dicarboxylate (+/−) Cis as a colorless oil (4.61 g, 67%).

LC/MS (AN01_001_012): Rt=1.89 min, 100%, [M+H]⁺=363.3.

¹H NMR (400 MHz, CDCl₃) δ 7.27-7.19 (m, 4H), 7.19-7.11 (m, 1H), 4.23-3.90 (m, 4H), 3.86 (d, J=13.2 Hz, 1H), 3.64 (d, J=13.2 Hz, 1H), 3.04 (s, 1H), 2.89-2.82 (m, 1H), 2.77 (s, 1H), 2.53 (dt, J=11.3, 4.1 Hz, 1H), 1.91-1.76 (m, 1H), 1.67-1.59 (m, 1H), 1.39 (s, 9H), 1.15 (t, J=7.1 Hz, 3H), one missing proton.

### 1-tert-butyl 4-ethyl (3S,4S)-(+/−)-cis-3-aminopiperidine-1,4-dicarboxylate

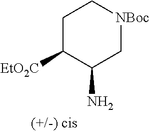 [SMILES: CCOC(=O)[C@H]1CCN(C(=O)OC(C)(C)C)C[C@H]1N]

To an argon-purged solution of 1-tert-butyl 4-ethyl (3S,4S)-(+/−)-cis-3-(benzylamino) piperidine-1,4-dicarboxylate (+/−) Cis (1 eq., 500 mg, 1.38 mmol) in methanol (5 mL) was added Pd (OH) 2 (0.15 eq., 145.28 mg, 0.207 mmol) at room temperature. The reaction mixture was purged with argon (×3) and then with H₂ (×3) and stirred at room temperature for 16 h. The reaction mixture was filtered through a pad of Celite. The cake was washed with DCM (100 mL). Distilled water (50 mL) was added. The two phases were separated and the aqueous layer was extracted with DCM (2×25 mL). The combined organic layers were dried over Na₂SO₄, filtered and concentrated to dryness under reduced pressure to afford 1-tert-butyl 4-ethyl (3S,4S)-(+/−)-cis-3-aminopiperidine-1,4-dicarboxylate (+/−) Cis as a yellowish oil (356 mg, 95%).

LC/MS (AN01_001_012): Rt=1.62 min, ND, [M+H]⁺=273.2.

¹H NMR (400 MHz, CDCl₃) δ 4.10 (q, J=7.2 Hz, 2H), 4.00-3.69 (m, 3H), 3.29 (s, 1H), 3.03-2.94 (m, 1H), 2.79 (t, J=12.4 Hz, 1H), 2.53-2.46 (m, 1H), 1.97-1.82 (m, 2H), 1.68 (s, 1H), 1.39 (s, 9H), 1.20 (t, J=7.1 Hz, 3H).

### 1-tert-butyl 4-ethyl (3S,4S)-(+/−)-cis-3-{[(tert-butoxy)carbonyl]amino}piperidine-1,4-dicarboxylate

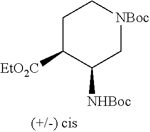 [SMILES: CCOC(=O)[C@H]1CCN(C(=O)OC(C)(C)C)C[C@H]1NC(=O)OC(C)(C)C]

1-tert-butyl 4-ethyl (3S,4S)-(+/−)-cis-3-aminopiperidine-1,4-dicarboxylate (+/−) Cis (1 eq., 356 mg, 1.307 mmol) was dissolved in DCM (5 mL) then Boc₂O (1.1 eq., 313.81 mg, 0.308 mL, 1.44 mmol) in DCM (5 mL) was added to the mixture at 0° C. The solution was stirred at room temperature for 16 h. Distilled water (50 mL) and DCM (50 mL) were added. The two phases were separated and the aqueous layer was extracted with DCM (2×50 mL). The combined organic layers were dried over Na₂SO₄, filtered and concentrated to dryness under reduced pressure. The crude was purified by flash chromatography over silica gel (15 μm, 40 g, dry loading, gradient: Cyclohexane:EtOAc from 100/0 to 60/40 over 21 CV) to afford 1-tert-butyl 4-ethyl (3S,4S)-(+/−)-cis-3-{[(tert-butoxy)carbonyl]amino}piperidine-1,4-dicarboxylate (+/−) Cis as a colorless oil (409 mg, 78%).

LC/MS (AN01_001_012): Rt=1.82 min, ND, [M−Boc+H]⁺=273.2.

¹H NMR (400 MHz, CDCl₃) δ 4.91 (d, J=9.2 Hz, 1H), 4.15-3.98 (m, 3H), 3.86-3.48 (m, 2H), 3.19-2.89 (m, 2H), 2.65 (dt, J=9.7, 4.2 Hz, 1H), 1.82 (dtd, J=13.9, 9.7, 4.1 Hz, 1H), 1.69 (dt, J=14.5, 4.6 Hz, 1H), 1.45-1.30 (m, 18H), 1.19 (t, J=7.1 Hz, 3H).

### (3S,4S)-(+/−)-cis-1-[(tert-butoxy)carbonyl]-3-{[(tert-butoxy)carbonyl]amino}piperidine-4-carboxylic acid

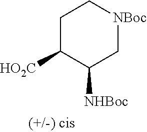 [SMILES: CC(C)(C)OC(=O)N[C@@H]1CN(C(=O)OC(C)(C)C)CC[C@@H]1C(=O)O]

To a solution of 1-tert-butyl 4-ethyl (3S,4S)-(+/−)-cis-3-{[(tert-butoxy)carbonyl]amino}piperidine-1,4-dicarboxylate (+/−) Cis (1 eq., 460 mg, 1.24 mmol) in THF (12.207 mL) was added a solution of LiOH (5 eq., 259.108 mg, 6.18 mmol) in water (6.46 mL) at room temperature and the reaction mixture was stirred at room temperature for 20 h. The reaction mixture was added dropwise at 0° C. to a stirred mixture of DCM (100 mL) and 1M HCl aqueous solution (50 mL). The two layers were separated and the aqueous layer was extracted with DCM (2×25 mL). The combined organic layers were dried over Na₂SO₄, filtered and concentrated to dryness under reduced pressure to afford (3S,4S)-(+/−)-cis-1-[(tert-butoxy)carbonyl]-3-{[(tert-butoxy)carbonyl]amino}piperidine-4-carboxylic acid (+/−) Cis as a colorless solid (424 mg, 90%).

¹H NMR (400 MHz, CDCl₃) δ 5.03 (d, J=8.9 Hz, 1H), 4.12-3.80 (m, 1H), 3.74 (s, 1H), 3.62 (s, 1H), 3.20 (d, J=13.2 Hz, 1H), 3.08 (s, 1H), 2.71 (s, 1H), 1.78-1.66 (m, 1H), 1.39 (s, 18H), 1.26-1.18 (m, 1H), one missing proton (COOH).

### tert-butyl (3S,4S)-(+/−)-cis-3-{[(tert-butoxy)carbonyl]amino}-4-{[(1S)-1-cyano-2-[4-(3-methyl-2-oxo-2,3-dihydro-1,3-benzoxazol-5-yl)phenyl]ethyl]carbamoyl}piperidine-1-carboxylate

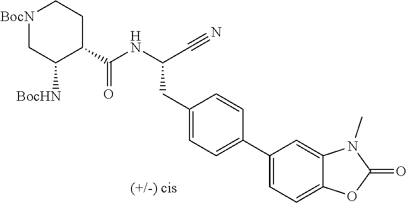 [SMILES: Cn1c(=O)oc2ccc(-c3ccc(C[C@@H](C#N)NC(=O)[C@H]4CCN(C(=O)OC(C)(C)C)C[C@H]4NC(=O)OC(C)(C)C)cc3)cc21]

Starting from (S)-2-amino-3-(4-(3-methyl-2-oxo-2,3-dihydrobenzo[d]oxazol-5-yl)phenyl)propanenitrile hydrochloride (1 eq., 150 mg, 0.45 mmol) and (3S,4S)-(+/−)-cis-1-[(tert-butoxy)carbonyl]-3-{[(tert-butoxy)carbonyl]amino}piperidine-4-carboxylic acid (+/−) Cis (1.67 eq., 290 mg, 0.76 mmol), using general procedure B, tert-butyl (3S,4S)-(+/−)-cis-3-{[(tert-butoxy)carbonyl]amino}-4-{[(1S)-1-cyano-2-[4-(3-methyl-2-oxo-2,3-dihydro-1,3-benzoxazol-5-yl)phenyl]ethyl]carbamoyl}piperidine-1-carboxylate (+/−) Cis was obtained as an orange solid (250 mg, 89%) after purification by flash chromatography over silica gel (50 μm, 40 g, dry loading, gradient: cyclohexane/EtOAc from 100/0 to 0/100 over 25 CV) and co-evaporation with DCM and n-hexane.

LC/MS (AN01_001_012): Rt=2.59 min, 58.3%, [M−Boc+H]⁺=520.3.

¹H NMR (400 MHz, CDCl₃) δ 7.61-7.53 (m, 2H), 7.48-7.42 (m, 1H), 7.42-7.37 (m, 1H), 7.34 (dt, J=8.3, 1.8 Hz, 1H), 7.28-7.25 (m, 1H), 7.13 (dd, J=6.7, 1.7 Hz, 1H), 5.20-4.98 (m, 2H), 4.10 (dd, J=43.1, 13.6 Hz, 2H), 3.46 (d, J=8.0 Hz, 3H), 3.19 (dd, J=7.3, 3.3 Hz, 1H), 3.15-3.10 (m, 1H), 3.02 (d, J=13.0 Hz, 1H), 2.94-2.83 (m, 1H), 2.62-2.47 (m, 1H), 2.17-1.94 (m, 1H), 1.85-1.63 (m, 1H), 1.54-1.42 (m, 18H), 1.35-1.30 (m, 1H), one missing proton.

### (3S,4S)-(+/−)-cis-3-amino-N-[(1S)-1-cyano-2-[4-(3-methyl-2-oxo-2,3-dihydro-1,3-benzoxazol-5-yl)phenyl]ethyl]piperidine-4-carboxamide IND-019 (+/−) Cis

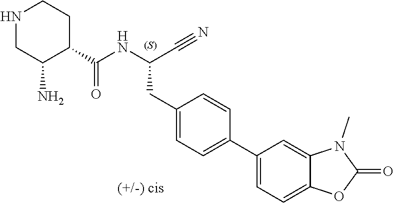

Starting from tert-butyl (3S,4S)-(+/−)-cis-3-{[(tert-butoxy)carbonyl]amino}-4-{[(1S)-1-cyano-2-[4-(3-methyl-2-oxo-2,3-dihydro-1,3-benzoxazol-5-yl)phenyl]ethyl]carbamoyl}piperidine-1-carboxylate (1 eq., 130 mg, 0.18 mmol) and using general procedure C, (3S,4S)-(+/−)-cis-3-amino-N-[(1S)-1-cyano-2-[4-(3-methyl-2-oxo-2,3-dihydro-1,3-benzoxazol-5-yl)phenyl]ethyl]piperidine-4-carboxamide was obtained as a white solid (36 mg, 47%) after purification by flash chromatography over silica gel (15 μm, 25 g, dry loading, gradient: DCM/(MeOH/NH4OH 9:1) from 100/0 to 80/20 over 33 CV), then preparative HPLC (Eluant: Water+0.2% ammonium bicarbonate/Acetonitrile, gradient: from 20 to 35% acetonitrile in water+0.2% ammonium bicarbonate, column: XBridge C18 (30×150 (5 μm)), flow Rate: 43 mL/min).

LC/MS (AN_01_001_026_DEDL): Rt=5.44 min, 95.7%, [M+H]⁺=420.7.

¹H NMR (400 MHz, DMSO) δ 9.25 (s, 1H), 7.73-7.63 (m, 2H), 7.61-7.55 (m, 1H), 7.46-7.30 (m, 4H), 5.01 (dt, J=11.1, 7.6 Hz, 1H), 3.41 (s, 3H), 3.21-3.06 (m, 2H), 2.91 (dd, J=13.4, 3.1 Hz, 1H), 2.81 (td, J=7.8, 3.7 Hz, 1H), 2.72-2.62 (m, 1H), 2.59-2.53 (m, 1H), 2.44-2.35 (m, 1H), 2.34-2.27 (m, 1H), 1.75 (d, J=76.4 Hz, 4H), 1.34 (dt, J=13.3, 3.3 Hz, 1H).

### Example 52. Synthesis of Compound 140

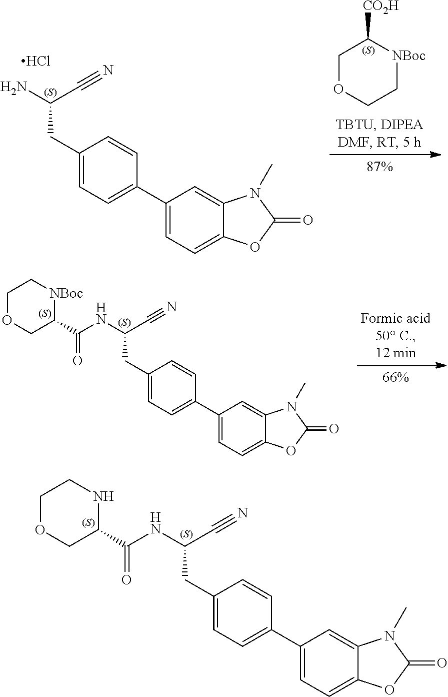

### tert-butyl (3S)-3-{[(1S)-1-cyano-2-[4-(3-methyl-2-oxo-2,3-dihydro-1,3-benzoxazol-5-yl)phenyl]ethyl]carbamoyl}morpholine-4-carboxylate

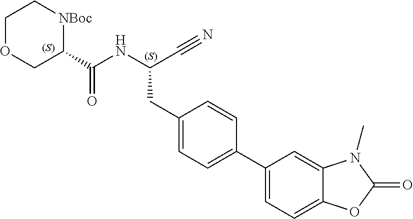

Starting from (S)-2-amino-3-(4-(3-methyl-2-oxo-2,3-dihydrobenzo[d]oxazol-5-yl)phenyl)propanenitrile (1 eq., 150 mg, 0.45 mmol) and (3S)-4-[(tert-butoxy)carbonyl]morpholine-3-carboxylic acid (1.1 eq., 115.701 mg, 0.5003 mmol), and using general procedure B, tert-butyl (3S)-3-{[(1S)-1-cyano-2-[4-(3-methyl-2-oxo-2,3-dihydro-1,3-benzoxazol-5-yl)phenyl]ethyl]carbamoyl}morpholine-4-carboxylate was obtained as an orange solid (200 mg, 87%) after purification by flash chromatography over silica gel (50 μm, 24 g, dry loading, gradient: cyclohexane/EtOAc from 100/0 to 100/0 over 20 CV) and co-evaporation with DCM and n-hexane.

LC/MS (AN01_001_012): Rt=2.42 min, 55%, [M−Boc+H]⁺=407.3.

¹H NMR (400 MHz, CDCl₃) δ 7.60-7.52 (m, 2H), 7.40-7.35 (m, 2H), 7.31 (dd, J=8.3, 1.7 Hz, 1H), 7.26 (d, J=8.2 Hz, 2H), 7.13 (d, J=1.8 Hz, 1H), 6.46 (s, 1H), 5.22 (q, J=6.9 Hz, 1H), 4.50-4.39 (m, 2H), 3.92-3.71 (m, 2H), 3.53 (dd, J=11.9, 4.0 Hz, 1H), 3.48-3.38 (m, 3H), 3.17 (d, J=6.6 Hz, 2H), 2.97-2.81 (m, 1H), 1.44 (s, 9H).

### (3S)—N-[(1S)-1-cyano-2-[4-(3-methyl-2-oxo-2,3-dihydro-1,3-benzoxazol-5-yl)phenyl]ethyl]morpholine-3-carboxamide

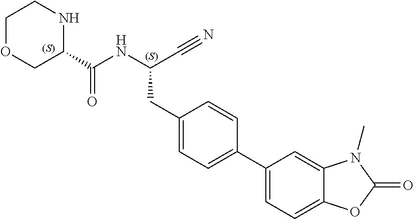

Starting from tert-butyl (3S)-3-{[(1S)-1-cyano-2-[4-(3-methyl-2-oxo-2,3-dihydro-1,3-benzoxazol-5-yl)phenyl]ethyl]carbamoyl}morpholine-4-carboxylate (1 eq., 200 mg, 0.39 mmol) and using general procedure C, (3S)—N-[(1S)-1-cyano-2-[4-(3-methyl-2-oxo-2,3-dihydro-1,3-benzoxazol-5-yl)phenyl]ethyl]morpholine-3-carboxamide was obtained as a white solid (106 mg, 66%) after purification by flash chromatography over silica gel (15 μm, 12 g, dry loading, gradient: Cyclohexane/EtOAc from 100/0 to 0/1 over 12 CV, 100% EtOAc over 22 CV and then EtOAc/MeOH 100/0 to 90/10 over 2 CV) and co-evaporation with MeOH and Et₂O.

LC/MS (AN_01_001_026_DEDL): Rt=6.26 min, 95.7%, [M+H]⁺=407.8.

¹H NMR (400 MHz, DMSO) δ 8.73-8.60 (m, 1H), 7.70-7.61 (m, 2H), 7.60-7.53 (m, 1H), 7.45-7.32 (m, 4H), 5.01 (q, J=8.0 Hz, 1H), 3.58 (dd, J=10.8, 3.5 Hz, 1H), 3.53 (dt, J=10.9, 3.5 Hz, 1H), 3.42-3.32 (m, 5H), 3.28 (dd, J=7.5, 3.5 Hz, 1H), 3.26-3.12 (m, 2H), 2.78-2.58 (m, 3H).

### Example 53. Synthesis of Compound 207

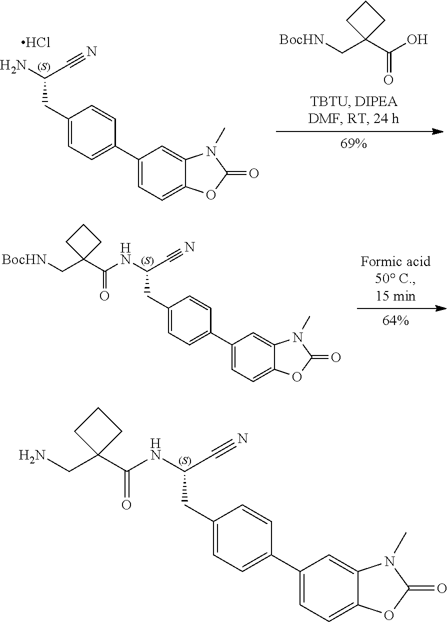

### tert-butyl N-[(1-{[(1S)-1-cyano-2-[4-(3-methyl-2-oxo-2,3-dihydro-1,3-benzoxazol-5-yl)phenyl]ethyl]carbamoyl}cyclobutyl)methyl]carbamate

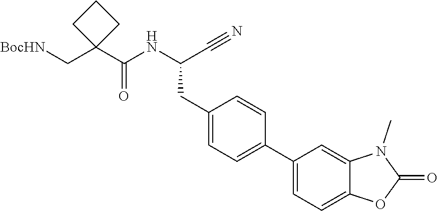 [SMILES: Cn1c(=O)oc2ccc(-c3ccc(C[C@@H](C#N)NC(=O)C4(CNC(=O)OC(C)(C)C)CCC4)cc3)cc21]

Starting from (S)-2-amino-3-(4-(3-methyl-2-oxo-2,3-dihydrobenzo[d]oxazol-5-yl)phenyl)propanenitrile (1 eq., 150 mg, 0.45 mmol) and 1-(((tert-butoxycarbonyl)amino)methyl)cyclobutane-1-carboxylic acid (1.05 eq., 109.5 mg, 0.48 mmol), and using general procedure B, tert-butyl N-[(1-{[(1S)-1-cyano-2-[4-(3-methyl-2-oxo-2,3-dihydro-1,3-benzoxazol-5-yl)phenyl]ethyl]carbamoyl}cyclobutyl)methyl]carbamate was obtained as an off-white solid (158 mg, 69%) after purification by flash chromatography over silica gel (50 μm, 24 g, cyclohexane/EtOAc from 100:0 to 40:60 over 40 min).

¹H NMR (400 MHz, DMSO) δ 8.45 (d, J=7.8 Hz, 1H), 7.71-7.65 (m, 2H), 7.60-7.57 (m, 1H), 7.45-7.36 (m, 4H), 6.70 (t, J=6.4 Hz, 1H), 4.97 (q, J=7.6 Hz, 1H), 3.41 (s, 3H), 3.23 (d, J=6.3 Hz, 2H), 3.15 (d, J=8.0 Hz, 2H), 2.18-2.02 (m, 2H), 1.95-1.87 (m, 2H), 1.81-1.72 (m, 1H), 1.67-1.58 (m, 1H), 1.37 (s, 9H).

### 1-(aminomethyl)-N-[(1S)-1-cyano-2-[4-(3-methyl-2-oxo-2,3-dihydro-1,3-benzoxazol-5-yl)phenyl]ethyl]cyclobutane-1-carboxamide

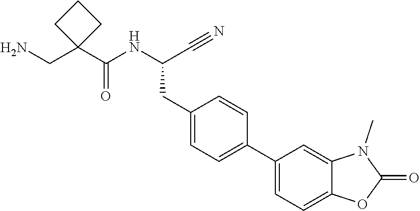 [SMILES: Cn1c(=O)oc2ccc(-c3ccc(C[C@@H](C#N)NC(=O)C4(CN)CCC4)cc3)cc21]

Starting from tert-butyl N-[(1-{[(1S)-1-cyano-2-[4-(3-methyl-2-oxo-2,3-dihydro-1,3-benzoxazol-5-yl)phenyl]ethyl]carbamoyl}cyclobutyl)methyl]carbamate (1 eq., 80 mg, 0.16 mmol) and using general procedure C, 1-(aminomethyl)-N-[(1S)-1-cyano-2-[4-(3-methyl-2-oxo-2,3-dihydro-1,3-benzoxazol-5-yl)phenyl]ethyl]cyclobutane-1-carboxamide was obtained as a white solid (41 mg, 64%) after purification by HPLC preparative (Eluant: Water+0.1% NH₄HCO₃/Acetonitrile, gradient: from 25 to 40% acetonitrile in water+0.1% BICAR in 23 min, column: XBridge C18 (30×150 (5 μm)), flow Rate: 43 mL/min).

LC/MS (AN_01_001_053): Rt=7.61 min, 98.1%, [M+H]⁺=405.9.

¹H NMR (400 MHz, DMSO) δ 8.85 (s, 1H), 7.70-7.63 (m, 2H), 7.58 (d, J=1.6 Hz, 1H), 7.45-7.36 (m, 4H), 5.03 (t, J=7.7 Hz, 1H), 3.41 (s, 3H), 3.22-3.08 (m, 2H), 2.76 (s, 2H), 2.18-2.07 (m, 2H), 1.88-1.20 (m, 6H).

### Example 54. Synthesis of Compound 143

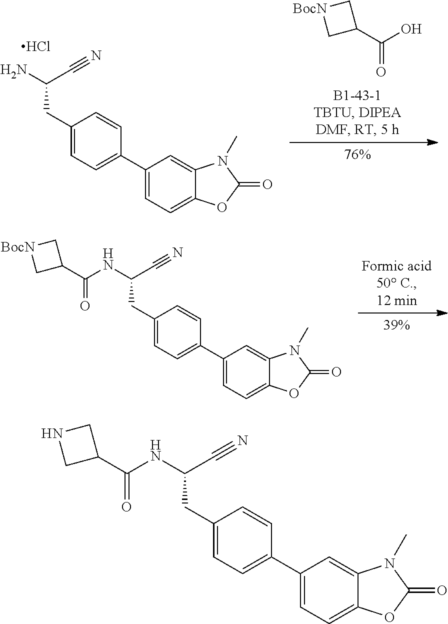 [SMILES: Cn1c(=O)oc2ccc(-c3ccc(C[C@H](N)C#N)cc3)cc21 ; Cl ; CC(C)(C)OC(=O)N1CC(C(=O)O)C1 ; Cn1c(=O)oc2ccc(-c3ccc(C[C@@H](C#N)NC(=O)C4CN(C(=O)OC(C)(C)C)C4)cc3)cc21 ; Cn1c(=O)oc2ccc(-c3ccc(C[C@@H](C#N)NC(=O)C4CNC4)cc3)cc21]

### tert-butyl 3-{[(1S)-1-cyano-2-[4-(3-methyl-2-oxo-2,3-dihydro-1,3-benzoxazol-5-yl)phenyl]ethyl]carbamoyl}azetidine-1-carboxylate

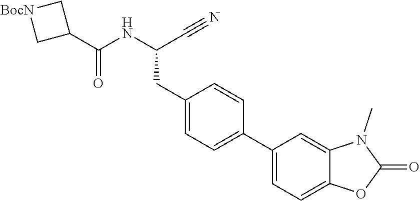 [SMILES: Cn1c(=O)oc2ccc(-c3ccc(C[C@@H](C#N)NC(=O)C4CN(C(=O)OC(C)(C)C)C4)cc3)cc21]

Starting from (S)-2-amino-3-(4-(3-methyl-2-oxo-2,3-dihydrobenzo[d]oxazol-5-yl)phenyl)propanenitrile (1 eq., 200 mg, 0.606 mmol) and 1-(tert-butoxycarbonyl) azetidine-3-carboxylic acid (1.1 eq., 134.24 mg, 0.67 mmol), and using general procedure B, tert-butyl 3-{[(1S)-1-cyano-2-[4-(3-methyl-2-oxo-2,3-dihydro-1,3-benzoxazol-5-yl)phenyl]ethyl]carbamoyl}azetidine-1-carboxylate was obtained as a white solid (219 mg, 76%) after purification by flash chromatography over silica gel (50 μm, 24 g, dry loading, gradient: cyclohexane/EtOAc from 100/0 to 10/90 over 27 CV).

LC/MS (AN01_001_012): Rt=2.38 min, 100%, [M−Boc+H]⁺=377.2.

¹H NMR (400 MHz, CDCl3) δ 7.50-7.42 (m, 2H), 7.31-7.26 (m, 2H), 7.23 (dd, J=8.3, 1.8 Hz, 1H), 7.17 (dd, J=8.3, 0.5 Hz, 1H), 7.00 (d, J=1.8 Hz, 1H), 6.21 (d, J=8.6 Hz, 1H), 5.20 (q, J=6.9 Hz, 1H), 4.10-3.89 (m, 4H), 3.33 (s, 3H), 3.19-3.03 (m, 3H), 1.36 (s, 9H).

### N-[(1S)-1-cyano-2-[4-(3-methyl-2-oxo-2,3-dihydro-1,3-benzoxazol-5-yl)phenyl]ethyl]azetidine-3-carboxamide

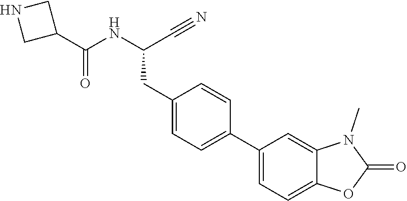 [SMILES: Cn1c(=O)oc2ccc(-c3ccc(C[C@@H](C#N)NC(=O)C4CNC4)cc3)cc21]

Starting from tert-butyl 3-{[(1S)-1-cyano-2-[4-(3-methyl-2-oxo-2,3-dihydro-1,3-benzoxazol-5-yl)phenyl]ethyl]carbamoyl}azetidine-1-carboxylate (1 eq., 100 mg, 0.21 mmol) and using general procedure C, N-[(1S)-1-cyano-2-[4-(3-methyl-2-oxo-2,3-dihydro-1,3-benzoxazol-5-yl)phenyl]ethyl]azetidine-3-carboxamide was obtained as a white solid (31 mg, 39%) after purification by preparative HPLC (XBridge prep C18, 150×30 mm, 5 μm, 43 ml/min, liquid loading, mobile phase gradient: water+0.2% NH₄HCO₃/ACN from 70/30 to 55/45 over 10 min).

LC/MS (AN_01_001_026_DEDL): Rt=6.30 min, 95.6%, [M+H]⁺=377.5.

¹H NMR (400 MHz, DMSO) δ 8.66 (d, J=7.8 Hz, 1H), 7.71-7.64 (m, 2H), 7.59 (d, J=1.7 Hz, 1H), 7.46-7.36 (m, 4H), 4.99 (q, J=7.8 Hz, 1H), 3.62 (t, J=6.7 Hz, 1H), 3.51 (t, J=6.6 Hz, 1H), 3.45-3.33 (m, 6H), 3.19-3.06 (m, 2H), 2.72 (s, 1H).

### Example 55. Synthesis of Compound 297

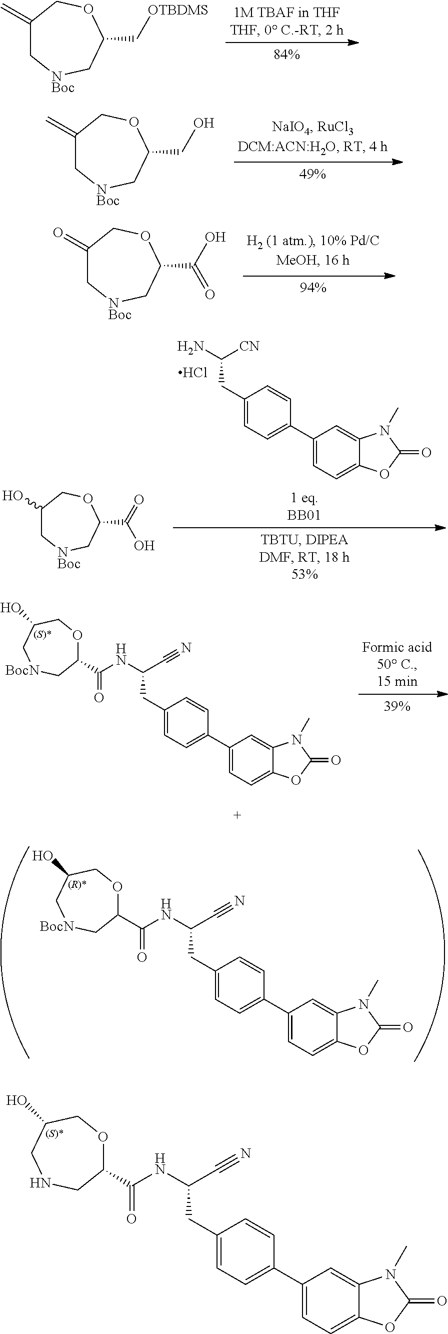

### tert-butyl (2S)-2-(hydroxymethyl)-6-methylidene-1,4-oxazepane-4-carboxylate

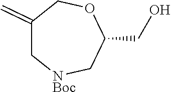 [SMILES: C=C1CO[C@H](CO)CN(C(=O)OC(C)(C)C)C1]

Starting from tert-butyl (S)-2-(((tert-butyldimethylsilyl)oxy)methyl)-6-methylene-1,4-oxazepane-4-carboxylate (1 eq., 1.90 g, 5.31 mmol) and using general procedure A, tert-butyl (2S)-2-(hydroxymethyl)-6-methylidene-1,4-oxazepane-4-carboxylate was obtained as a colorless oil (1.09 g, 84%) after purification by silica gel flash chromatography (25 g, gradient: Cyclohexane/EtOAc from 95:5 to 50:50).

LC/MS (AN01_001_012): Rt=2.04 min, 100%, [M+Na]⁺=266.2.

### (2S)-4-[(tert-butoxy)carbonyl]-6-oxo-1,4-oxazepane-2-carboxylic acid

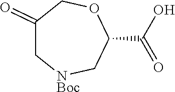 [SMILES: CC(C)(C)OC(=O)N1CC(=O)CO[C@H](C(=O)O)C1]

To a solution of (2S)-4-[(tert-butoxy)carbonyl]-6-oxo-1,4-oxazepane-2-carboxylic acid (1 eq., 440 mg, 1.81 mmol) in a mixture of DCM (5 mL), ACN (7.5 mL) and H₂O (4 mL) were added sodium periodate (5 eq., 1.93 g, 9.04 mmol) and RuCl₃ (0.2 eq. 75.0 mg, 0.36 mmol) at room temperature. The resulting mixture was vigorously stirred at room temperature for 4 h. The reaction mixture was diluted with DCM (20 mL) and a saturated aqueous solution of NaHCO₃ (20 mL). The two layers were separated, and the aqueous layer was washed with DCM (20 mL). The aqueous layer was then acidified with an aqueous solution of 3M HCl until pH ˜1 and extracted with DCM (2×20 mL). The combined organic layers were dried over Na₂SO₄, filtered and concentrated under reduced pressure to afford (2S)-4-[(tert-butoxy)carbonyl]-6-oxo-1,4-oxazepane-2-carboxylic acid as a colorless oil (225 mg, 48%).

LC/MS (AN01_001_012): Rt=1.88 min, non-UV active [M−H]−=258.1.

### (2S)-4-[(tert-butoxy)carbonyl]-6-hydroxy-1,4-oxazepane-2-carboxylic acid

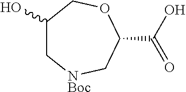 [SMILES: CC(C)(C)OC(=O)N1CC(O)CO[C@H](C(=O)O)C1]

To an argon purged solution of (2S)-4-[(tert-butoxy)carbonyl]-6-oxo-1,4-oxazepane-2-carboxylic acid (1 eq., 240 mg, 0.926 mmol) in MeOH (9.3 mL) was added 10% Pd/C (0.2 eq., 197 mg, 0.185 mmol) at room temperature. The resulting mixture was purged with argon (×3) and then with H₂ (3×). The reaction mixture was stirred under an atmospheric pressure of H₂ at room temperature for 18 h. The reaction mixture was purged with argon, filtered on a pad of celite and rinsed with MeOH (2×10 mL). The filtrate was concentrated under reduced pressure to afford (2S)-4-[(tert-butoxy)carbonyl]-6-hydroxy-1,4-oxazepane-2-carboxylic acid as a white solid (227 mg, 94%, diastereomeric mixture).

LC/MS (AN01_001_012): Rt=1.76 min, non-UV active, [M−H]−=260.1.

### tert-butyl (2S,6S*)-2-{[(1S)-1-cyano-2-[4-(3-methyl-2-oxo-2,3-dihydro-1,3-benzoxazol-5-yl)phenyl]ethyl]carbamoyl}-6 hydroxy-1,4-oxazepane-4-carboxylate and tert-butyl (2S,6R*)-2-{[(1S)-1-cyano-2-[4-(3-methyl-2-oxo-2,3-dihydro-1,3-benzoxazol-5-yl)phenyl]ethyl]carbamoyl}-6-hydroxy-1,4-oxazepane-4-carboxylate

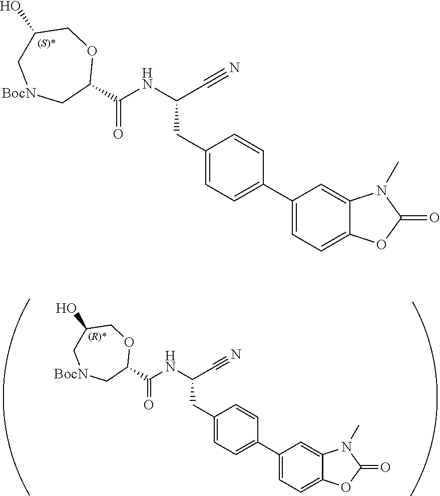

Starting from (S)-2-amino-3-(4-(3-methyl-2-oxo-2,3-dihydrobenzo[d]oxazol-5-yl)phenyl)propanenitrile (1 eq., 264 mg, 0.802 mmol) and (2S)-4-[(tert-butoxy)carbonyl]-6-hydroxy-1,4-oxazepane-2-carboxylic acid (1.05 eq., 220 mg, 0.842 mmol), using general procedure B, the obtained diastereomeric mixture was separated by purification on silica gel flash chromatography (25 g, gradient: DCM/MeOH from 100:0 to 96:4) affording tert-butyl (2S,6S*)-2-{[(1S)-1-cyano-2-[4-(3-methyl-2-oxo-2,3-dihydro-1,3-benzoxazol-5-yl)phenyl]ethyl]carbamoyl}-6 hydroxy-1,4-oxazepane-4-carboxylate (100 mg, 23%) and B1-(2S,6R*)-2-{[(1S)-1-cyano-2-[4-(3-methyl-2-oxo-2,3-dihydro-1,3-benzoxazol-5-yl)phenyl]ethyl]carbamoyl}-6-hydroxy-1,4-oxazepane-4-carboxylate (121 mg, 28%) as pale yellow solids. The stereochemistry (S)* was arbitrarily assigned to the first eluted product by flash chromatography and then the second eluted product was assigned (R)*.

LC/MS (AN01_001_012): Rt=2.30 min, 100%, [M−C₄H₈+H]⁺=481.2.

LC/MS (AN01_001_012): Rt=2.30 min, 100%, [M−C₄H₈+H]⁺=481.2.

### (2S,6S*)—N-[(1S)-1-cyano-2-[4-(3-methyl-2-oxo-2,3-dihydro-1,3-benzoxazol-5-yl)phenyl]ethyl]-6-hydroxy-1,4-oxazepane-2-carboxamide

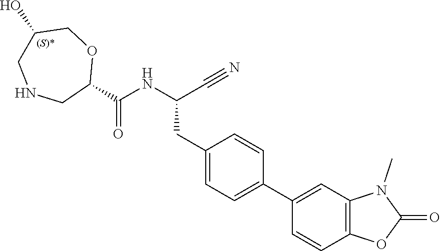 [SMILES: Cn1c(=O)oc2ccc(-c3ccc(C[C@@H](C#N)NC(=O)[C@@H]4CNC[C@H](O)CO4)cc3)cc21]

(2S,6S*)—N-[(1S)-1-cyano-2-[4-(3-methyl-2-oxo-2,3-dihydro-1,3-benzoxazol-5-yl)phenyl]ethyl]-6-hydroxy-1,4-oxazepane-2-carboxamide was prepared using general procedure C, starting from tert-butyl (2S,6S*)-2-{[(1S)-1-cyano-2-[4-(3-methyl-2-oxo-2,3-dihydro-1,3-benzoxazol-5-yl)phenyl]ethyl]carbamoyl}-6 hydroxy-1,4-oxazepane-4-carboxylate (1 eq., 50.0 mg, 0.093 mmol). The crude residue was purified by preparative HPLC (gradient: H₂O (+0.1% TFA)/Acetonitrile, from 85:15 to 70:30, column: XBridge C18 (30×150 (5 μm)), Flow Rate: 43 mL/min). The collected fractions containing (2S,6S*)—N-[(1S)-1-cyano-2-[4-(3-methyl-2-oxo-2,3-dihydro-1,3-benzoxazol-5-yl)phenyl]ethyl]-6-hydroxy-1,4-oxazepane-2-carboxamide were combined and the organic solvent was removed under reduced pressure. The resulting aqueous layer was basified with solid NaHCO₃ until pH˜8 and extracted with DCM (3×30 mL). The combined organic layers were dried over Na₂SO₄, filtered and concentrated under reduced pressure. The resulting residue was dissolved in a H₂O/MeOH mixture (90:10, 2 mL) and freeze-dried to afford (2S,6S*)—N-[(1S)-1-cyano-2-[4-(3-methyl-2-oxo-2,3-dihydro-1,3-benzoxazol-5-yl)phenyl]ethyl]-6-hydroxy-1,4-oxazepane-2-carboxamide as a white solid (15.7 mg, 39%).

LC/MS (AN01_001_026): Rt=6.44 min, 100%, [M+H]⁺=437.6.

¹H NMR (DMSO-d₆, 400 MHz): δ ppm 8.61 (d, J=8.4 Hz, 1H), 7.66 (d, J=8.3 Hz, 2H), 7.58-7.56 (m, 1H), 7.41-7.37 (m, 4H), 5.02 (q, J=8.0 Hz, 1H), 4.76 (d, J=5.3 Hz, 1H), 4.00 (dd, J=7.7, 4.1 Hz, 1H), 3.88 (dd, J=12.3, 4.8 Hz, 1H), 3.76-3.69 (m, 1H), 3.47 (dd, J=12.2, 7.8 Hz, 1H), 3.41 (s, 3H), 3.25-3.15 (m, 2H), 3.03 (dd, J=14.2, 4.0 Hz, 1H), 2.81 (dd, J=13.7, 3.8 Hz, 1H), 2.62 (dd, J=13.7, 5.9 Hz, 1H), 2.54-2.50 (m, 2H).

### Example 56. Synthesis of Compound 296

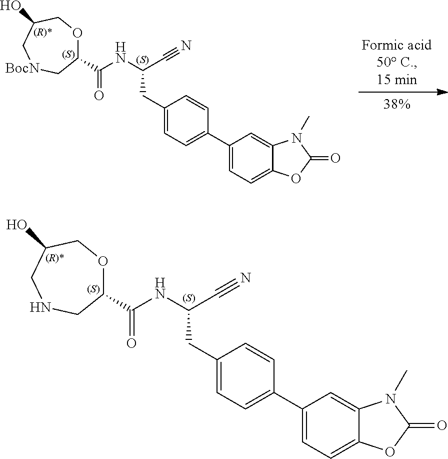

### (2S,6R*)—N-[(1S)-1-cyano-2-[4-(3-methyl-2-oxo-2,3-dihydro-1,3-benzoxazol-5-yl)phenyl]ethyl]-6-hydroxy-1,4-oxazepane-2-carboxamide

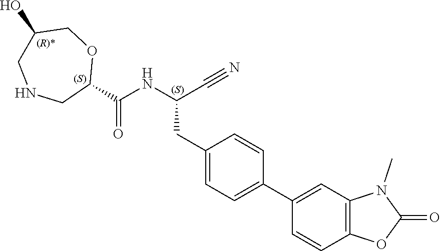

Starting from (2S,6R*)-2-{[(1S)-1-cyano-2-[4-(3-methyl-2-oxo-2,3-dihydro-1,3-benzoxazol-5-yl)phenyl]ethyl]carbamoyl}-6-hydroxy-1,4-oxazepane-4-carboxylate prepared as described in Example 55 procedure (1 eq., 60 mg, 0.11 mmol) and using the general procedure C, (2S,6R*)—N-[(1S)-1-cyano-2-[4-(3-methyl-2-oxo-2,3-dihydro-1,3-benzoxazol-5-yl)phenyl]ethyl]-6-hydroxy-1,4-oxazepane-2-carboxamide was obtained as a white solid (18.4 mg, 38%) after preparative HPLC (Eluant: Water+0.1% TFA/Acetonitrile, gradient: from 15 to 30% acetonitrile in water+0.1% TFA in 28 min, column: XBridge C18 (30×150 (5 μm), flow Rate: 43 mL/min) and evaporation of ACN. Then, NaHCO₃ (solid) was added to the aqueous solution (pH ˜7-8). The aqueous layer was extracted with DCM (3×30 mL). The combined organic layers were dried over Na₂SO₄, filtered and concentrated to dryness under reduced. Finally, the residue was freeze-dried overnight.

LC/MS (AN01_001_026): Rt=6.37 min, 100%, [M+H]⁺=437.5.

¹H NMR (400 MHz, DMSO) δ 8.67 (d, J=8.5 Hz, 1H), 7.69-7.60 (m, 2H), 7.60-7.55 (m, 1H), 7.45-7.35 (m, 4H), 5.02 (td, J=8.6, 7.3 Hz, 1H), 4.75 (d, J=5.3 Hz, 1H), 3.93 (dd, J=8.9, 4.3 Hz, 1H), 3.82-3.70 (m, 2H), 3.70-3.63 (m, 1H), 3.40 (s, 3H), 3.25-3.14 (m, 2H), 3.06 (dd, J=14.0, 4.3 Hz, 1H), 2.93 (dd, J=13.3, 4.8 Hz, 1H), 2.47-2.40 (m, 2H), one missing proton (NH).

### Example 57. Synthesis of Compound 245:2-(1-aminocyclohexyl)-N-[(1S)-1-cyano-2-[4-(3-methyl-2-oxo-2,3-dihydro-1,3-benzoxazol-5-yl)phenyl]ethyl]acetamide

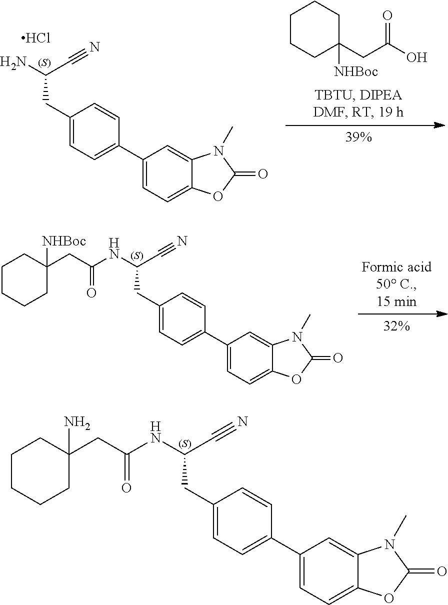

### tert-butyl N-[1-({[(1S)-1-cyano-2-[4-(3-methyl-2-oxo-2,3-dihydro-1,3-benzoxazol-5-yl)phenyl]ethyl]carbamoyl}methyl)cyclohexyl]carbamate

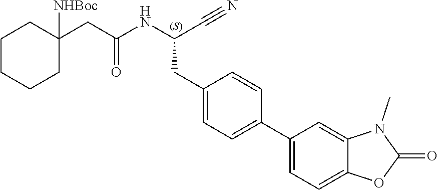

Starting from (S)-2-amino-3-(4-(3-methyl-2-oxo-2,3-dihydrobenzo[d]oxazol-5-yl)phenyl)propanenitrile (1 eq., 150 mg, 0.45 mmol) and 2-(1-{[(tert-butoxy)carbonyl]amino}cyclohexyl) acetic acid (1.05 eq., 122.9 mg, 0.48 mmol), and using general procedure B, tert-butyl N-[1-({[(1S)-1-cyano-2-[4-(3-methyl-2-oxo-2,3-dihydro-1,3-benzoxazol-5-yl)phenyl]ethyl]carbamoyl}methyl)cyclohexyl]carbamate was obtained as a white solid (88 mg, 39%) after purification by flash chromatography over silica gel (12 g, dry loading (silica), AcOEt in Cyclohexane 0-80% in 20 min+25 min at 80%).

LC/MS (AN01_001_012): Rt=2.65 min, 100%, [M−Boc+H]⁺=433.2

¹H NMR (400 MHz, DMSO) δ 8.64 (d, J=7.9 Hz, 1H), 7.69-7.63 (m, 2H), 7.57 (d, J=1.6 Hz, 1H), 7.45-7.36 (m, 4H), 6.20 (s, 1H), 5.00 (q, J=7.9 Hz, 1H), 3.40 (s, 3H), 3.15 (dd, J=13.6, 7.1 Hz, 1H), 3.07 (dd, J=13.6, 8.6 Hz, 1H), 2.48-2.37 (m, 2H), 1.98-1.88 (m, 2H), 1.37 (s, 9H), 1.36-1.20 (m, 7H), 1.12-1.02 (m, 1H).

### 2-(1-aminocyclohexyl)-N-[(1S)-1-cyano-2-[4-(3-methyl-2-oxo-2,3-dihydro-1,3-benzoxazol-5-yl)phenyl]ethyl]acetamide

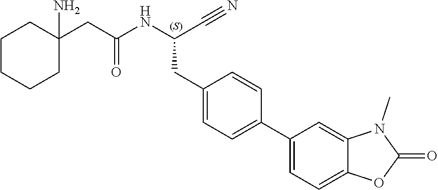

Starting from tert-butyl N-[1-({[(1S)-1-cyano-2-[4-(3-methyl-2-oxo-2,3-dihydro-1,3-benzoxazol-5-yl)phenyl]ethyl]carbamoyl}methyl)cyclohexyl]carbamate (1 eq., 117 mg, 0.19 mmol) and using general procedure C, 2-(1-aminocyclohexyl)-N-[(1S)-1-cyano-2-[4-(3-methyl-2-oxo-2,3-dihydro-1,3-benzoxazol-5-yl)phenyl]ethyl]acetamide was obtained as a white solid (33 mg, 32%) after purification by preparative HPLC (Eluant: Water+0.1% TFA/Acetonitrile; gradient: from 25 to 40% acetonitrile in water+0.1% TFA; Column: XBridge C18 (30×150 (5 μm))), then MeCN was evaporated in vaccuo (30° C.) and the aqueous layer was basified with sodium bicarbonate to pH=8 and extracted with DCM (3×100 mL). The organic layer was dried with Na₂SO₄ and evaporated to dryness. This oil was freeze-dried using Acetonitrile/water (1:10).

LC/MS (AN_01_001_021_DEDL): Rt=7.39 min, 99.1%, [M+H]⁺=433.5.

¹H NMR (400 MHz, DMSO) δ 9.14 (s, 1H), 7.69-7.65 (m, 2H), 7.59-7.56 (m, 1H), 7.44-7.39 (m, 4H), 5.04 (t, J=7.7 Hz, 1H), 3.41 (s, 3H), 3.18 (dd, J=13.6, 6.9 Hz, 1H), 3.10 (dd, J=13.6, 8.5 Hz, 1H), 2.16 (s, 2H), 1.46 (s, 2H), 1.37-1.17 (m, 10H).

### Example 59. Synthesis of Compound 246

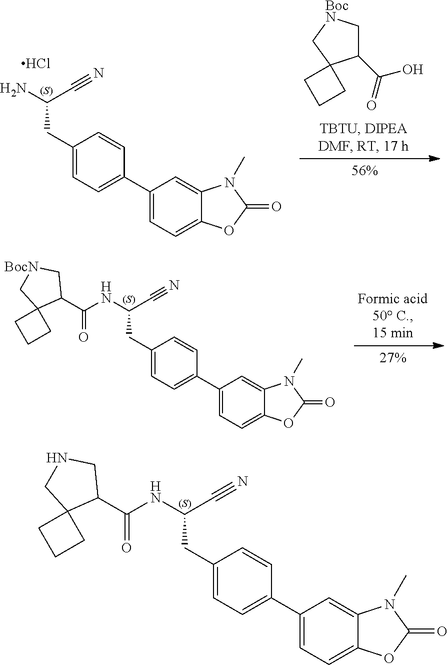

### tert-butyl 8-{[(1S)-1-cyano-2-[4-(3-methyl-2-oxo-2,3-dihydro-1,3-benzoxazol-5-yl)phenyl]ethyl]carbamoyl}-6-azaspiro[3.4]octane-6-carboxylate

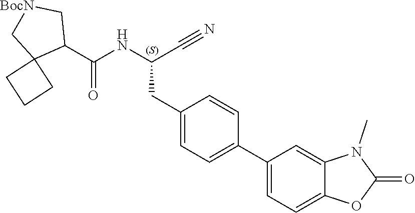

Starting from (S)-2-amino-3-(4-(3-methyl-2-oxo-2,3-dihydrobenzo[d]oxazol-5-yl)phenyl)propanenitrile (1 eq., 150 mg, 0.45 mmol) and 6-[(tert-butoxy)carbonyl]-6-azaspiro[3.4]octane-8-carboxylic acid (1.05 eq., 121.94 mg, 0.48 mmol), and using general procedure B, tert-butyl 8-{[(1S)-1-cyano-2-[4-(3-methyl-2-oxo-2,3-dihydro-1,3-benzoxazol-5-yl)phenyl]ethyl]carbamoyl}-6-azaspiro[3.4]octane-6-carboxylate was obtained as an yellowish gum (140 mg, 56%) after purification by flash chromatography over silica gel ((irregular SiOH, 50 μm, 25 g Interchim, dry loading (silica), mobile phase gradient: cyclohexane/EtOAc from 90/10 to 40/60 over 30 min) and co-evaporation with DCM.

LC/MS (AN01_001_012): Rt=2.57 min, 100%, [M−Boc+H]⁺=431.2

¹H NMR (400 MHz, DMSO) δ 8.98-8.88 (m, 1H), 7.67 (t, J=8.3 Hz, 2H), 7.61-7.54 (m, 1H), 7.48-7.35 (m, 4H), 5.12-4.99 (m, 1H), 3.43-3.34 (m, 4H), 3.28-3.08 (m, 5H), 2.82-2.70 (m, 1H), 2.07-1.95 (m, 1H), 1.91-1.71 (m, 3H), 1.61 (d, J=12.7 Hz, 1H), 1.51-1.43 (m, 1H), 1.43-1.29 (m, 9H).

### N-[(1S)-1-cyano-2-[4-(3-methyl-2-oxo-2,3-dihydro-1,3-benzoxazol-5-yl)phenyl]ethyl]-6-azaspiro[3.4]octane-8-carboxamide

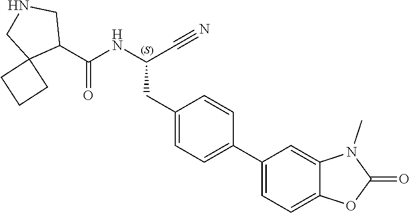

Starting from tert-butyl 8-{[(1S)-1-cyano-2-[4-(3-methyl-2-oxo-2,3-dihydro-1,3-benzoxazol-5-yl)phenyl]ethyl]carbamoyl}-6-azaspiro[3.4]octane-6-carboxylate (1 eq., 0.15 g, 0.22 mmol) and using general procedure C, N-[(1S)-1-cyano-2-[4-(3-methyl-2-oxo-2,3-dihydro-1,3-benzoxazol-5-yl)phenyl]ethyl]-6-azaspiro[3.4]octane-8-carboxamide was obtained as a white solid (25.3 mg, 27%) after purification by preparative HPLC preparative HPLC (XBridge prep C18, 150×30 mm, 5 μm, 43 mL/min, liquid loading, mobile phase gradient: water+0.2% NH₄CO₃/MeCN from 80/20 to 65/35 over 18 min) and freeze-drying using acetonitrile/water (1:10).

LC/MS (AN_01_001_021_DEDL): Rt=7.26 min, 97.2%, [M+H]⁺=431.4.

¹H NMR (400 MHz, DMSO) δ 8.78 (dd, J=24.4, 7.9 Hz, 1H), 7.74-7.61 (m, 2H), 7.61-7.52 (m, 1H), 7.52-7.30 (m, 4H), 5.11-4.95 (m, 1H), 3.40 (d, J=3.2 Hz, 3H), 3.28-3.07 (m, 2H), 2.94-2.66 (m, 4H), 2.58-2.53 (m, 1H), 2.48-2.42 (m, 1H), 2.04-1.94 (m, 1H), 1.89-1.52 (m, 3H), 2H are overlapping with solvents peaks.

### Example 60. Synthesis of Compound 180 ((1R,3S,5R)—N—((S)-1-cyano-2-(4-(3-methyl-2-oxo-2,3-dihydrobenzo[d]oxazol-5-yl)phenyl)ethyl)-2-azabicyclo[3.1.0]hexane-3-carboxamide)

Compound 180 was synthesized according to the scheme below, and the final product was isolated in 3.5 g with high purity (99.2% purity by HPLC-UV, 99.0% enantiomeric excess) and overall yield of 19.2% over 8 steps.

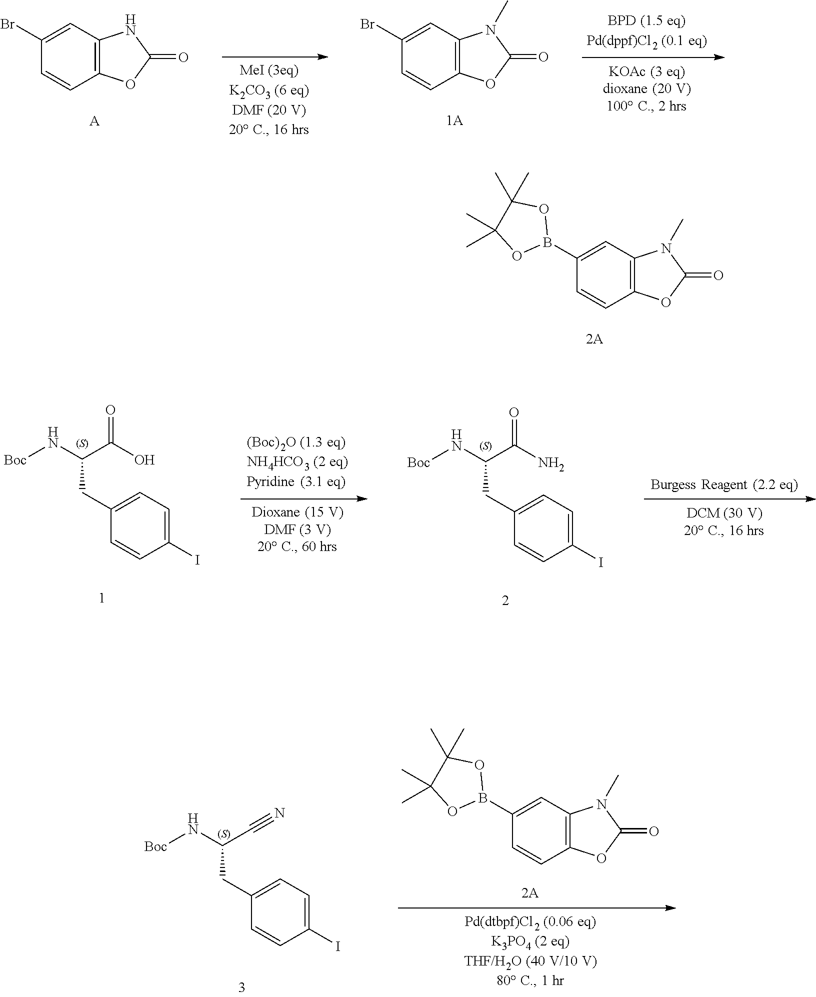

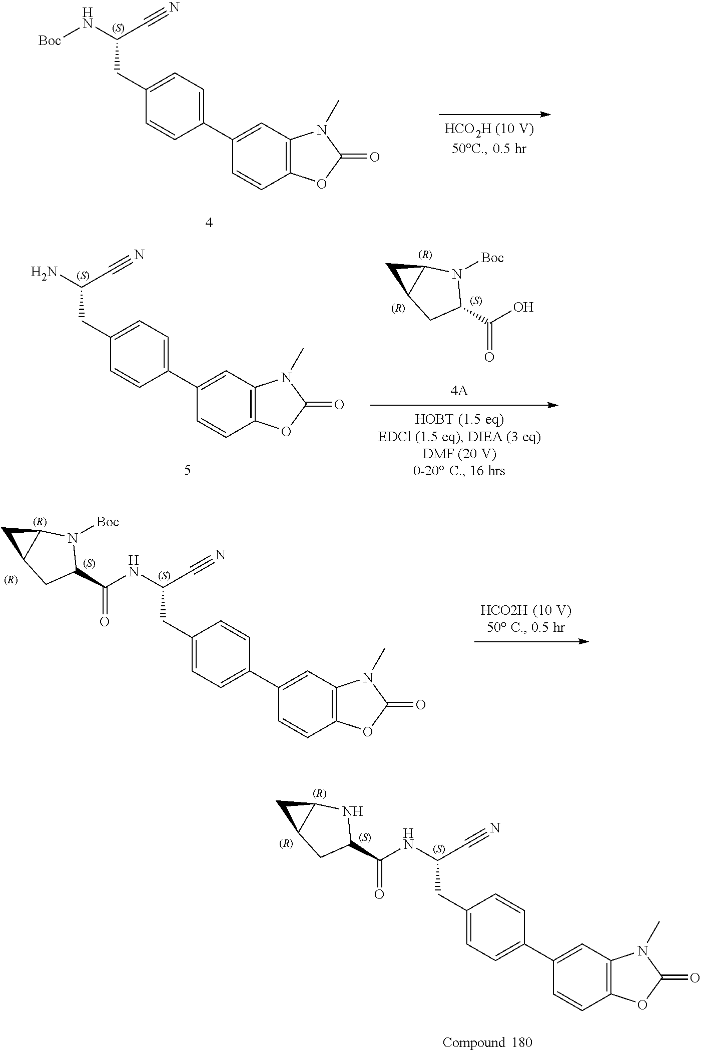

### Example 61. Synthesis of compound 309: (25,3aS,6aS)—N—((S)-1-cyano-2-(4′-cyano-[1,1′-biphenyl]-4-yl)ethyl)octahydrocyclopenta[b]pyrrole-2-carboxamide

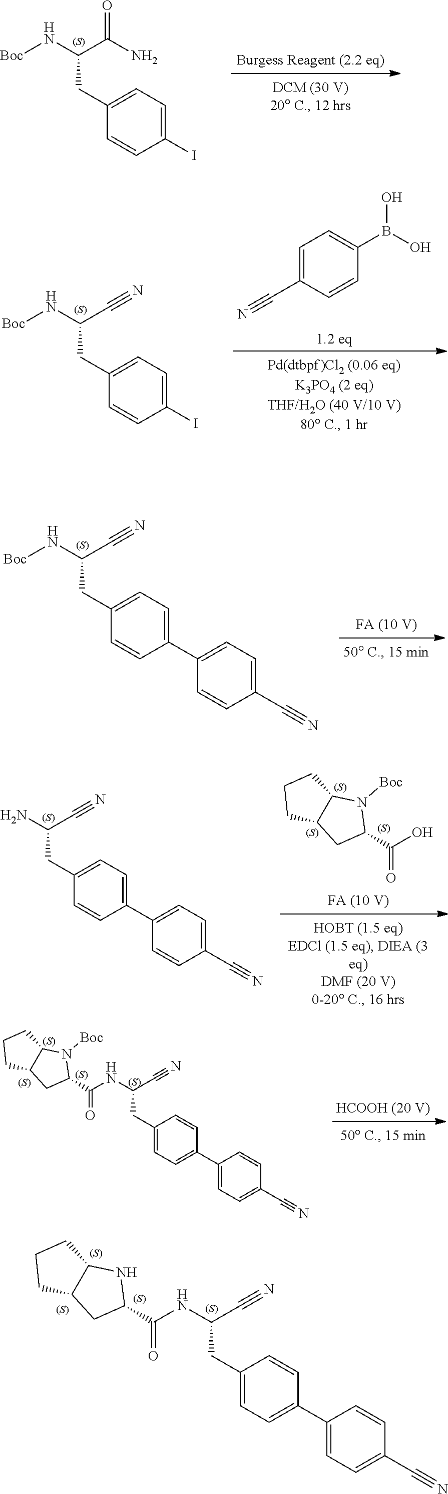

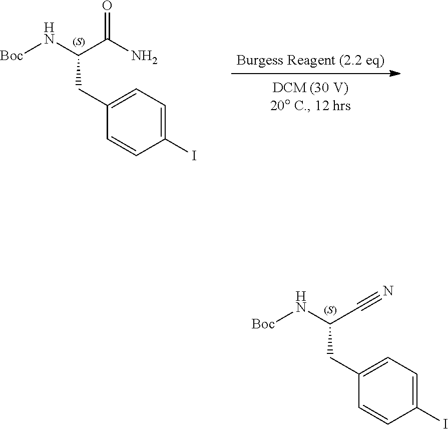

To a solution of tert-butyl (S)-(1-amino-3-(4-iodophenyl)-1-oxopropan-2-yl) carbamate (10 g, 25.63 mmol) in dichloromethane (300 mL) was added Burgess Reagent (13.44 g, 56.38 mmol) at 20° C. The reaction mixture was stirred at 20° C. for 12 hours. LC-MS showed starting material was consumed and 92.8% of desired product was detected. The reaction mixture was poured into water (200 mL) and extracted with dichloromethane (3×100 mL). The organic layer was washed with brine (200 mL), dried over Na₂SO₄, filtered and concentrated under reduced pressure to give crude product. The crude product was purified by column chromatography (SiO₂, Petroleum ether/Ethyl acetate=100/0 to 84/16) to give tert-butyl (S)-(1-cyano-2-(4-iodophenyl)ethyl) carbamate (8.14 g, 85.34% yield) as a white solid.

¹H NMR: (400 MHz, DMSO-d₆)

δ ppm 7.68 (d, J=8.25 Hz, 2H) 7.12 (d, J=8.13 Hz, 2H) 4.64 (q, J=8.09 Hz, 1H) 2.93-3.05 (m, 2H) 1.35 (s, 9H) 1.17 (t, J=7.32 Hz, 5H)

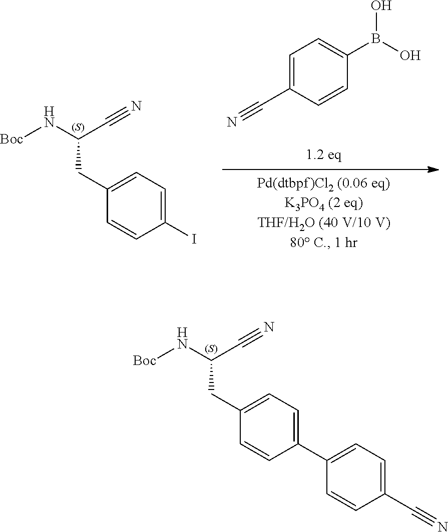

To a solution of tert-butyl (S)-(1-cyano-2-(4-iodophenyl)ethyl) carbamate (500 mg, 1.34 mmol) and (4-cyanophenyl) boronic acid (236.87 mg, 1.61 mmol) in tetrahydrofuran (20 mL) was added K₃PO₄ (570.30 mg, 2.69 mmol) in H₂O (5 mL) at 20° C. The vessel was evacuated then backfilled with N₂. This process was repeated three times. Then ditert-butyl(cyclopentyl)phosphane; dichloropalladium; iron (52.53 mg, 80.60 μmol) was added to the solution at 20° C. The vessel was evacuated then backfilled with N₂. This process was repeated three times. The mixture was stirred for 1 hour at 80° C. LC-MS showed starting material was consumed and 85.6% of product with desired Ms was detected. The reaction mixture was poured into water (30 mL) and extracted with ethyl acetate (3×20 mL). The organic layer was washed with brine (35 mL), dried over Na₂SO₄, filtered and concentrated under reduced pressure to give crude product which was purified by column chromatography (SiO₂, Petroleum ether/Ethyl acetate=19/1 to 10/1) to give tert-butyl (S)-(1-cyano-2-(4′-cyano-[1,1′-biphenyl]-4-yl)ethyl) carbamate (260 mg, 55.71% yield) as a white solid.

¹H NMR: ET69981-4-P1A (400 MHz, DMSO-d₆)

δ=7.87-7.94 (m, 4H) 7.73 (d, J=8.13 Hz, 2H) 7.45 (br d, J=8.13 Hz, 2H) 4.71 (q, J=7.92 Hz, 1H) 3.04-3.17 (m, 2H) 1.35 (s, 9H)

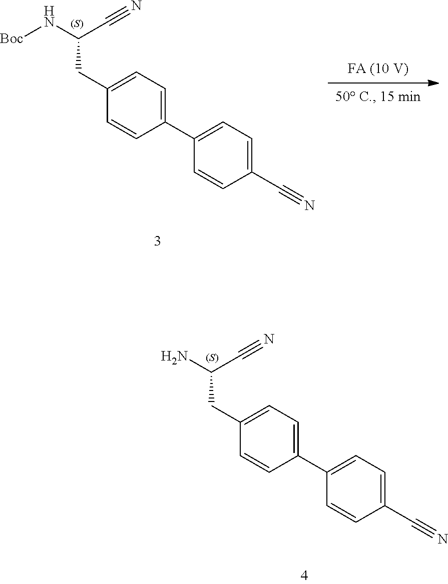

A solution of tert-butyl (S)-(1-cyano-2-(4′-cyano-[1,1′-biphenyl]-4-yl)ethyl) carbamate (0.114 g, 328.14 μmol) in HCOOH (1.2 mL) was stirred at 50° C. for 15 mins. LC-MS showed the reaction was completed. The reaction mixture was concentrated under reduced pressure to give(S)-4′-(2-amino-2-cyanoethyl)-[1,1′-biphenyl]-4-carbonitrile (0.07 g, 86.26% yield) as a yellow solid.

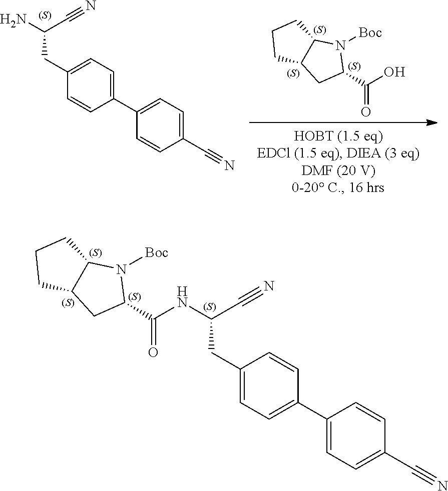

To a solution of (2S,3aS,6aS)-1-(tert-butoxycarbonyl) octahydrocyclopenta[b]pyrrole-2-carboxylic acid (72.27 mg, 283.06 μmol) in dimethylformamide (1 mL) was added N, N-Diisopropylethylamine (109.75 mg, 849.19 μmol, 147.91 μL), 1-(3-dimethylaminopropyl)-3-ethylcarbodiimide Hydrochloride (81.40 mg, 424.60 μmol) and hydroxybenzotriazole (57.37 mg, 424.60 μmol) at 20° C. Then a solution of (S)-4′-(2-amino-2-cyanoethyl)-[1,1′-biphenyl]-4-carbonitrile (0.07 g, 283.06 μmol) in dimethylformamide (1 mL) was added to the above solution at 0° C. The mixture was stirred at 20° C. for 16 hours. LC-MS showed starting material was consumed completely and desired product mass was detected. The reaction mixture was quenched by addition H₂O (10 mL) at 20° C., and extracted with ethyl acetate (20 mL×3). The combined organic layers were washed with water (20 mL) and brine (20 mL), dried over Na₂SO₄, filtered and concentrated under reduced pressure to give a residue which was purified by flash silica gel chromatography (ISCO®; 12 g SepaFlash® Silica Flash Column, Eluent of 0˜25% ethyl acetate/petroleum ether gradient @ 40 mL/min) to give tert-butyl (2S,3aS,6aS)-2-(((S)-1-cyano-2-(4′-cyano-[1,1′-biphenyl]-4-yl)ethyl) carbamoyl) hexahydrocyclopenta[b]pyrrole-1 (2H)-carboxylate (0.045 g, 32.81% yield) as a white solid.

¹H NMR: (400 MHz, CDCl₃-d₆)

δ ppm 7.70-7.82 (m, 2H) 7.62-7.70 (m, 2H) 7.56 (br d, J=7.50 Hz, 2H) 7.38 (br d, J=7.63 Hz, 2H) 7.27 (s, 1H) 5.16 (br s, 1H) 4.34 (br s, 1H) 4.15 (br d, J=7.13 Hz, 1H) 3.16 (br dd, J=12.57, 6.69 Hz, 2H) 2.61 (br s, 1H) 2.08-2.49 (m, 2H) 1.56-1.96 (m, 4H) 1.46 (br s, 11H)

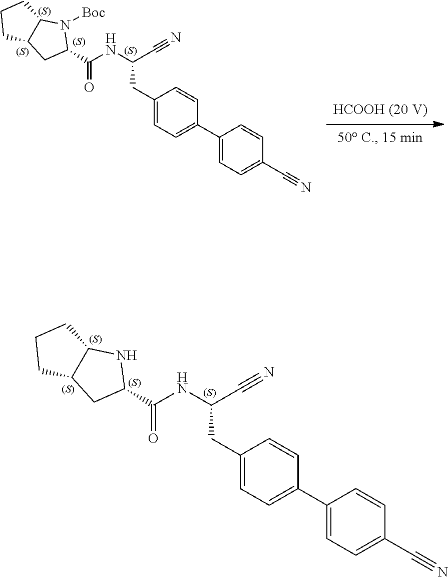

A solution of tert-butyl (2S,3aS,6aS)-2-(((S)-1-cyano-2-(4′-cyano-[1,1′-biphenyl]-4-yl)ethyl) carbamoyl) hexahydrocyclopenta[b]pyrrole-1 (2H)-carboxylate (45 mg, 92.86 μmol) in HCOOH (1 mL) was stirred at 50° C. for 15 mins. LC-MS showed starting material was consumed completely and product with desired mass was detected. The reaction mixture was concentrated under reduced pressure to give a residue which was purified by prep-HPLC to give (2S,3aS,6aS)—N—((S)-1-cyano-2-(4′-cyano-[1,1′-biphenyl]-4-yl)ethyl)octahydrocyclopenta[b]pyrrole-2-carboxamide (14.1 mg, 36.33% yield, 92.0% purity) as a white solid.

Column: Waters Xbridge Prep OBD C18 150*40 mm*10 um; mobile phase: [H₂O (10 mM NH₄HCO₃)—CH₃CN]; gradient: 35%-60% B over 8.0 min.

¹H NMR: (400 MHz, DMSO-d6)

δ ppm 8.40 (d, J=8.50 Hz, 1H) 7.89-7.96 (m, 2H) 7.84-7.89 (m, 2H) 7.70 (d, J=8.13 Hz, 2H) 7.42 (d, J=8.13 Hz, 2H) 5.09 (q, J=8.17 Hz, 1H) 3.55 (br s, 1H) 3.24 (br d, J=7.50 Hz, 2H) 2.67 (br s, 1H) 2.31-2.44 (m, 1H) 1.95-2.07 (m, 1H) 1.45-1.63 (m, 2H) 1.32-1.45 (m, 2H) 1.29-1.66 (m, 1H) 1.23 (s, 1H) 1.13 (br d, J=10.38 Hz, 1H) 0.79-0.94 (m, 1H)

### Example 62. Synthesis of compound 181: (1S,3S,5S)—N—((S)-1-cyano-2-(4-(3-methyl-2-oxo-2,3-dihydrobenzo[d]oxazol-5-yl)phenyl)ethyl)-2-azabicyclo[3.1.0]hexane-3-carboxamide

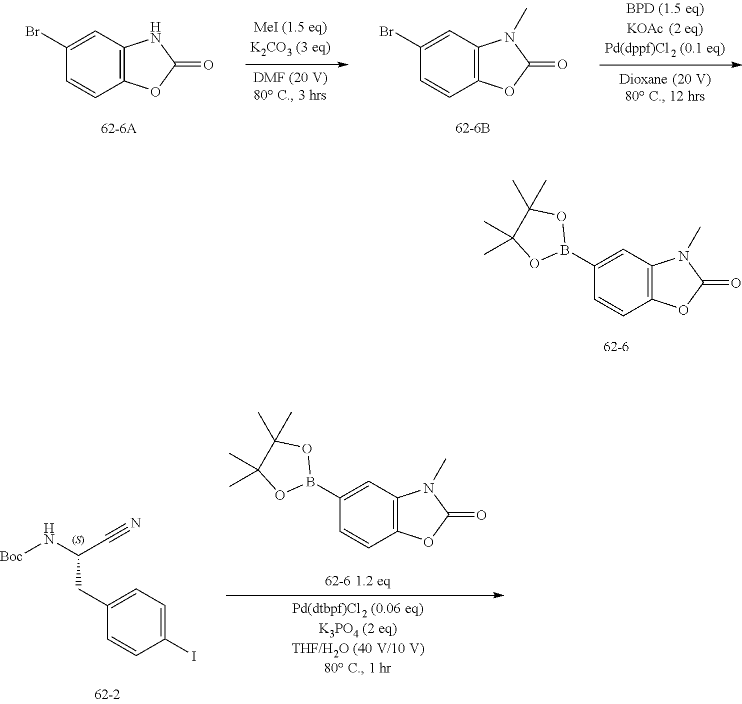

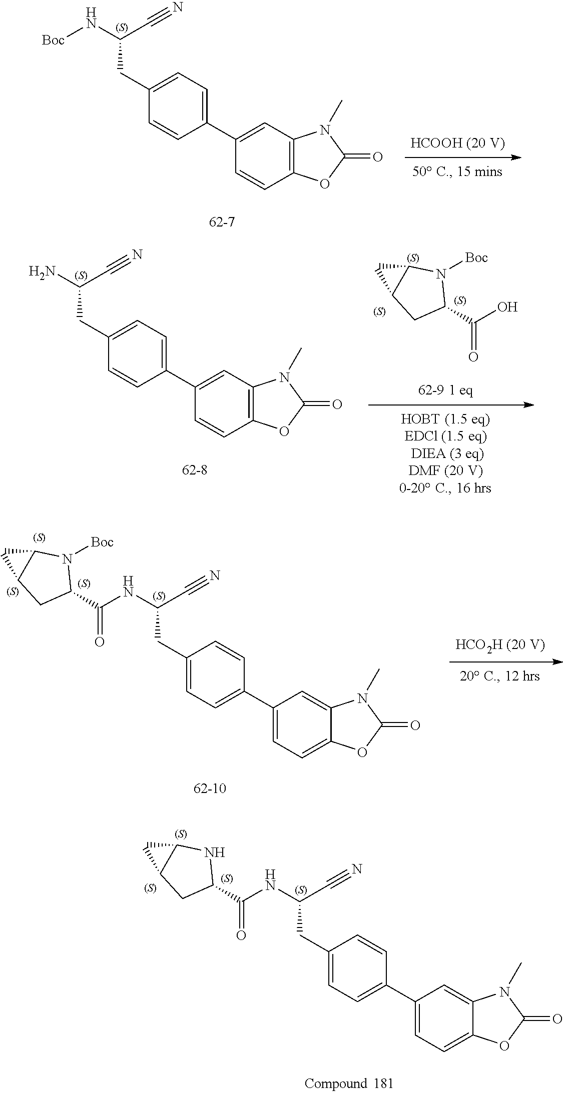

### General Procedure for Preparation of Compound 62-6B

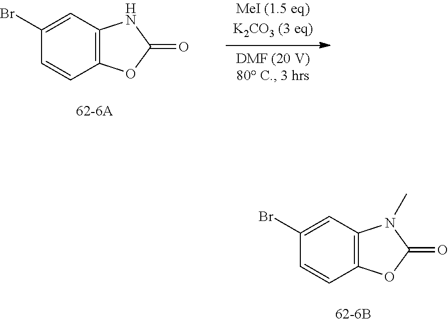

To a solution of Compound 62-6A (0.55 g, 2.57 mmol) in Dimethyl Formamide (11 mL) was added methyl iodide (547.16 mg, 3.85 mmol) and K₂CO₃ (1.07 g, 7.71 mmol) at 20° C. The mixture was stirred for 3 hours at 80° C. LC-MS showed starting material was consumed and 50.7% of desired product was detected. The reaction mixture was poured into water (20 mL) extracted with ethyl acetate (3×20 mL). The organic layer was washed with brine (35 mL), dried over Na₂SO₄, filtered and concentrated under reduced pressure to give crude product. The crude product was purified by column chromatography (SiO₂, Petroleum ether/Ethyl acetate=25/1 to 15/1) to give Compound 62-6B (560 mg, 95.56% yield) as a red solid.

¹H NMR: (400 MHz, DMSO-d₆)

δ ppm 7.56 (s, 1H) 7.29 (d, J=1.13 Hz, 2H) 3.33 (s, 3H)

### General Procedure for Preparation of Compound 62-6

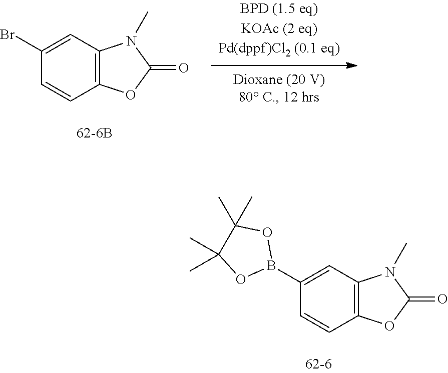 [SMILES: Cn1c(=O)oc2ccc(Br)cc21 ; Cn1c(=O)oc2ccc(B3OC(C)(C)C(C)(C)O3)cc21]

To a solution of Compound 62-6B (510 mg, 2.24 mmol) in Dioxane (10.2 mL) was added 4,4,5,5-tetramethyl-2-(4,4,5,5-tetramethyl-1,3,2-dioxaborolan-2-yl)-1,3,2-dioxaborolane (851.87 mg, 3.35 mmol) and KOAc (438.98 mg, 4.47 mmol) at 20° C. The vessel was evacuated then backfilled with N₂. [1,1-Bis(diphenylphosphino)ferrocene]dichloropalladium(II) (163.64 mg, 223.64 μmol) was added to the solution at 20° C. The vessel was evacuated then backfilled with N₂. This process was repeated 3 times. The mixture was stirred for 12 hours at 80° C. LC-MS showed starting material was consumed and 51.8% of desired product was detected. The reaction mixture was filtered through a pad of celite and the filtrate was concentrated under reduced pressure to give crude product. The crude product was purified by column chromatography (SiO₂, Petroleum ether/Ethyl acetate=25/1 to 9/1) to give Compound 62-6 (600 mg, 97.52% yield) as a yellow solid.

¹H NMR: (400 MHz, DMSO-d₆)

δ=7.40-7.52 (m, 2H) 7.33 (br d, J=7.88 Hz, 1H) 3.36 (s, 3H) 1.15 (s, 12H)

### General Procedure for Preparation of Compound 62-7

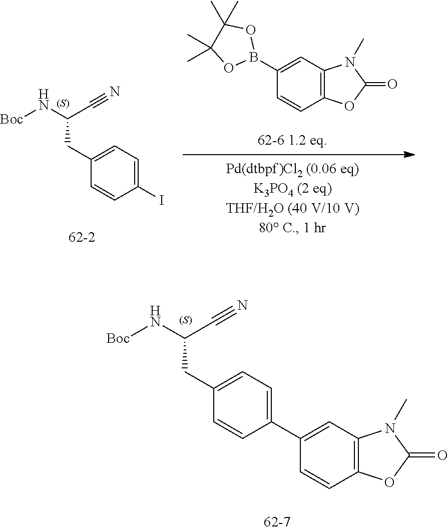

To a solution of Compound 62-2 (620 mg, 1.67 mmol) and Compound 62-6 (549.92 mg, 2.00 mmol) in tetrahydrofuran (24.8 mL) was added K₃PO₄ (707.17 mg, 3.33 mmol) in H₂O (3.1 mL) at 20° C. The vessel was evacuated then backfilled with N₂. This process was repeated 3 times. Ditert-butyl(cyclopentyl)phosphane; dichloropalladium; iron (65.14 mg, 99.95 μmol) was added to the solution at 20° C. The vessel was evacuated then backfilled with N₂. This process was repeated 3 times. The mixture was stirred for 1 hour at 80° C. LC-MS showed 1.4% of starting material was remained and 85.6% of desired product was detected. The reaction mixture was poured into ice water (30 mL) and extracted with ethyl acetate (3×10 mL). The organic layer was washed with brine (10 mL), dried over Na₂SO₄, filtered and concentrated under reduced pressure to give crude product. The crude product was purified by column chromatography (SiO₂ Petroleum ether/Ethyl acetate-9/1 to 3/1) to give Compound 62-7 (470 mg, 71.72% yield) as a brown solid.

¹H NMR: (400 MHz, DMSO-d₆)

δ=7.85 (br d, J=7.75 Hz, 1H) 7.63-7.69 (m, 2H) 7.58 (d, J=1.00 Hz, 1H) 7.36-7.44 (m, 4H) 4.69 (q, J=7.46 Hz, 1H) 3.40 (s, 3H) 3.09 (br d, J=7.75 Hz, 2H) 1.37 (s, 8H) 1.28-1.46 (m, 1H)

### General Procedure for Preparation of Compound 62-8

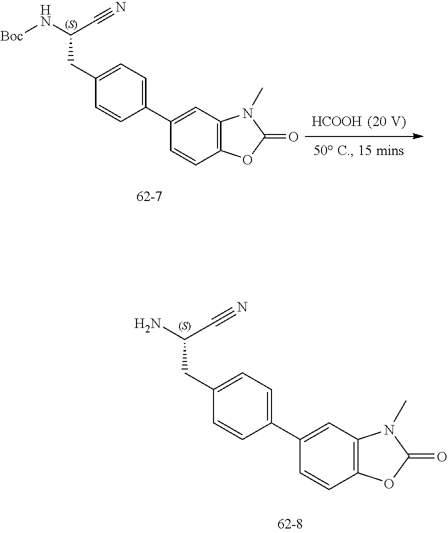

A solution of Compound 7 (0.05 g, 127.09 μmol) in HCOOH (1.0 mL) was stirred at 50° C. for 0.25 hour. LC-MS showed starting material was consumed and 54.5% of desired product was detected. The reaction mixture was concentrated in vacuum under 20° C. to give Compound 8 (35 mg, 93.89% yield) as a white solid.

¹H NMR: (400 MHz, DMSO-d₆)

δ=8.14 (s, 1H) 7.66 (d, J=8.13 Hz, 2H) 7.59 (d, J=0.75 Hz, 1H) 7.35-7.45 (m, 3H) 3.99 (dd, J=8.25, 6.63 Hz, 1H) 3.40 (s, 3H) 2.85-3.05 (m, 2H)

### General Procedure for Preparation of Compound 62-10

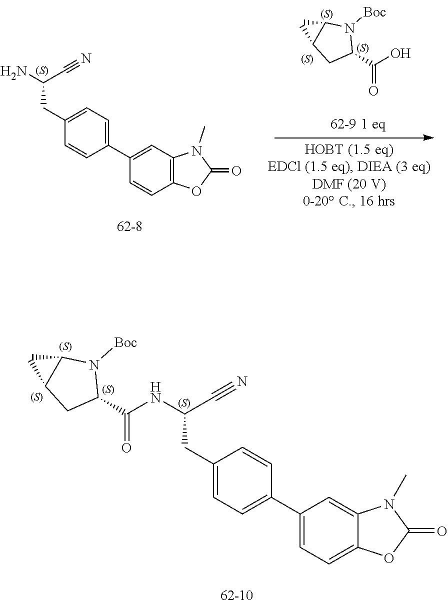

To a solution of Compound 62-9 (46.49 mg, 204.56 μmol) in Dimethyl Formamide (1.2 mL) was added diisopropylethylamine (79.31 mg, 613.67 μmol) at 20° C. 1-hydroxybenzotriazole (41.46 mg, 306.83 μmol) and 3-(ethyliminomethylideneamino)-N,N-dimethylpropan-1-amine (58.82 mg, 306.83 μmol) was added to the solution at 0° C. The solution was stirred at 0° C. for 0.5 hour. Compound 62-8 (60 mg, 204.56 μmol) was added to the solution at 0° C. The solution was stirred at 20° C. for 12 hours. LC-MS showed starting material was consumed and 53.8% of desired product was detected. The reaction mixture was poured into water (10 mL) and extracted with ethyl acetate (3×5 mL). The organic layer was washed with brine (5 mL), dried over Na₂SO₄, filtered and concentrated under reduced pressure to give crude product. The crude product was purified by prep-TLC (ethyl acetate: methyl alcohol=1:1) to give Compound 62-10 (78 mg, 75.87% yield) as a white solid.

¹H NMR: (400 MHz, DMSO-d₆)

δ=8.67-8.77 (m, 1H) 7.66 (d, J=8.13 Hz, 2H) 7.56-7.60 (m, 1H) 7.38-7.44 (m, 4H) 4.93 (q, J=7.88 Hz, 1H) 4.29-4.48 (m, 1H) 3.40 (s, 3H) 3.16 (br d, J=7.88 Hz, 2H) 1.61-1.75 (m, 1H) 1.38-1.52 (m, 5H) 1.24 (s, 9H) 0.85 (br s, 1H) 0.60-0.67 (m, 1H)

### General Procedure for Preparation of (1S,3S,5S)—N—((S)-1-cyano-2-(4-(3-methyl-2-oxo-2,3-dihydrobenzo[d]oxazol-5-yl)phenyl)ethyl)-2-azabicyclo[3.1.0]hexane-3-carboxamide

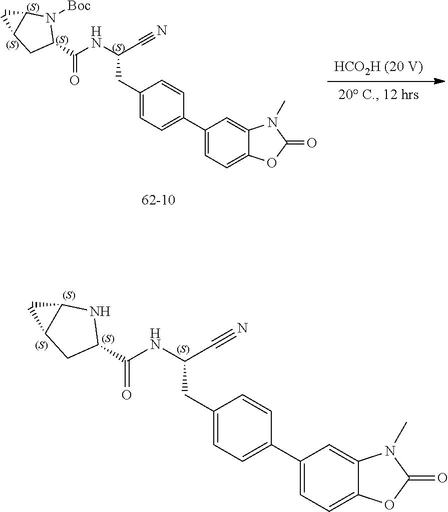

A solution of Compound 62-10 (73 mg, 145.26 μmol) in HCOOH (1.5 mL) was stirred at 20° C. for 12 hours. LC-MS showed starting material was consumed. Several new peaks were shown on LC-MS and 94.5% of desired product was detected. The reaction mixture was concentrated under reduced pressure to give the crude product. The crude product was purified by prep-HPLC and lyophilized to give: 1S,3S,5S)—N—((S)-1-cyano-2-(4-(3-methyl-2-oxo-2,3-dihydrobenzo[d]oxazol-5-yl)phenyl)ethyl)-2-azabicyclo[3.1.0]hexane-3-carboxamide (16.1 mg, 97.3% purity) as a white solid.

Column: Waters Xbridge Prep OBD C18 150*40 mm*10 um; mobile phase: [H₂O (10 mM NH₄HCO₃)-ACN]; gradient: 25%-55% B over 8.0 mins

¹H NMR: (400 MHz, DMSO-d₆)

δ=8.52 (d, J=8.25 Hz, 1H) 7.66 (d, J=8.25 Hz, 2H) 7.57 (s, 1H) 7.33-7.45 (m, 4H) 4.99 (q, J=7.92 Hz, 1H) 3.76 (br t, J=5.94 Hz, 1H) 3.40 (s, 3H) 3.21 (br d, J=7.88 Hz, 2H) 2.71-2.78 (m, 1H) 1.90-2.01 (m, 2H) 1.12-1.22 (m, 1H) 0.11-0.22 (m, 1H)-0.53- −0.42 (m, 1H)

### Example 63. Synthesis of Compound 187: (2S,3aS,6aR)—N—((S)-1-cyano-2-(4-(3-methyl-2-oxo-2,3-dihydrobenzo[d]oxazol-5-yl)phenyl)ethyl)hexahydro-1H-furo[3,4-b]pyrrole-2-carboxamide

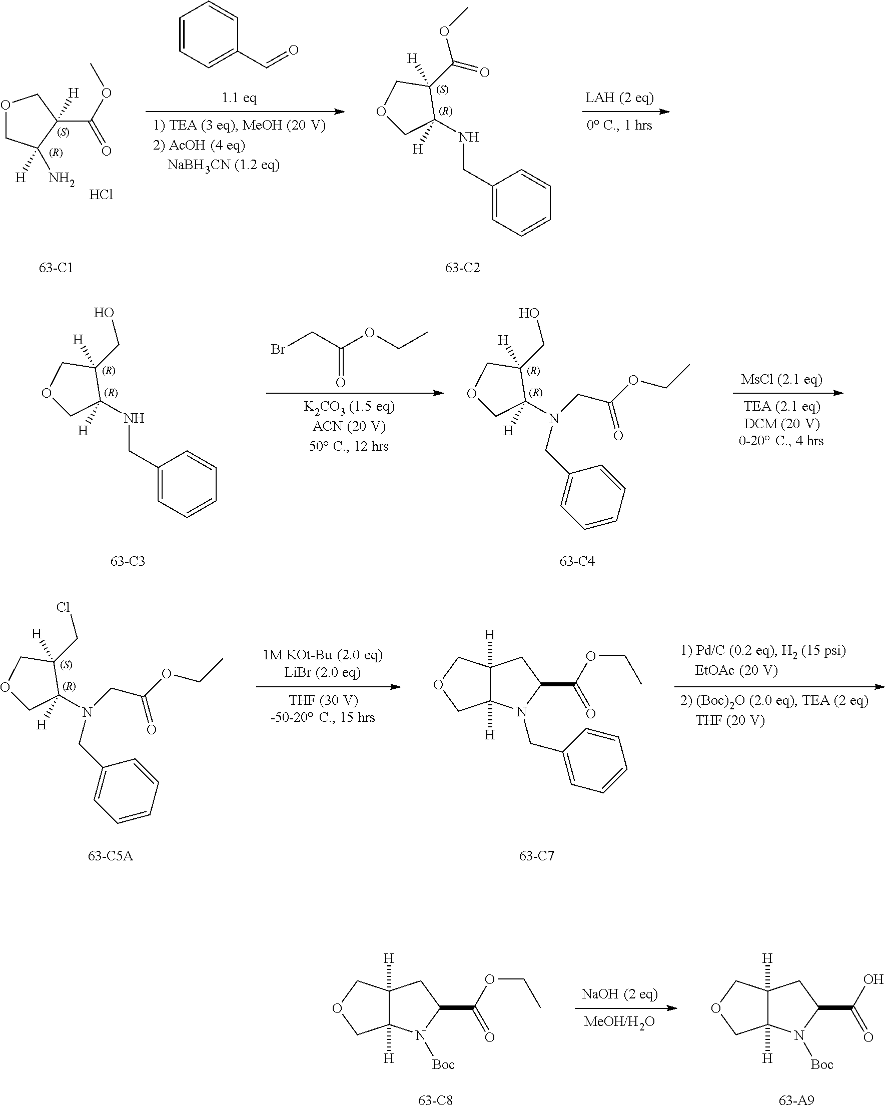

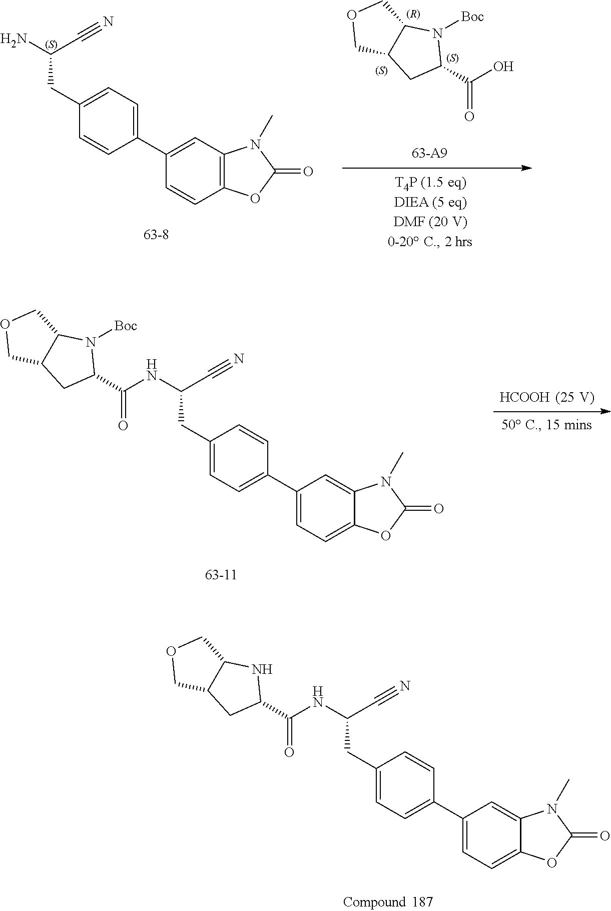

### General Procedure for Preparation of Compound 63-C2

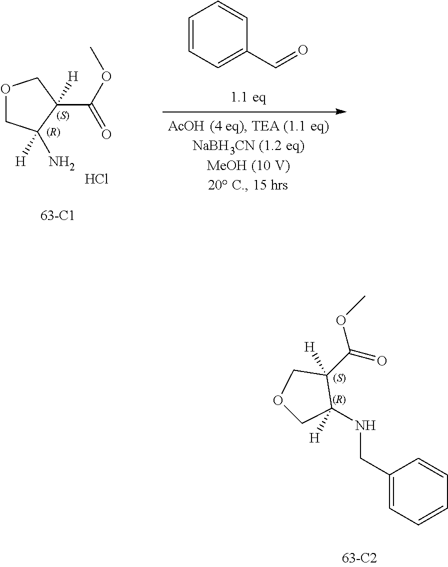

To a solution of compound 63-C1 (2.0 g, 11.01 mmol) in MeOH (10 mL) was added TEA (1.23 g, 12.11 mmol, 1.69 mL) and stirred at 20° C. for 30 mins. Then AcOH (2.65 g, 44.05 mmol, 2.52 mL) was added to the solution and stirred at 20° C. for 15 mins. Then was added benzaldehyde (1.17 g, 11.01 mmol, 1.11 mL) and stirred at 20° C. for 2 hrs. Then was added NaBH₃CN (830.43 mg, 13.21 mmol) to the solution at 20° C. The mixture was stirred at 20° C. for 12 hrs. TLC showed the reaction was completed. The reaction mixture was poured into water (50 mL), extracted with ethyl acetate (3×50 mL), the combined organic layer was washed with brine (50 mL), dried over anhydrous Na₂SO₄, filtered, concentrated under reduced pressure to give crude product. The residue was purified by flash silica gel chromatography (ISCO®; 12 g SepaFlash® Silica Flash Column, Eluent of 0˜25% Ethylacetate/Petroleum ethergradient @ 36 mL/min) to give compound 63-C2 (2.0 g, 77.19% yield) as a colorless oil.

¹H NMR: (400 MHz, DMSO-d₆)

δ 7.25-7.34 (m, 4H) 7.18-7.25 (m, 1H) 3.95 (dd, J=8.57, 6.57 Hz, 1H) 3.81-3.88 (m, 1H) 3.66-3.76 (m, 3H) 3.64 (s, 3H) 3.53-3.59 (m, 1H) 3.46 (dt, J=7.35, 5.46 Hz, 1H) 3.22-3.30 (m, 1H)

### General Procedure for Preparation of Compound 63-C3

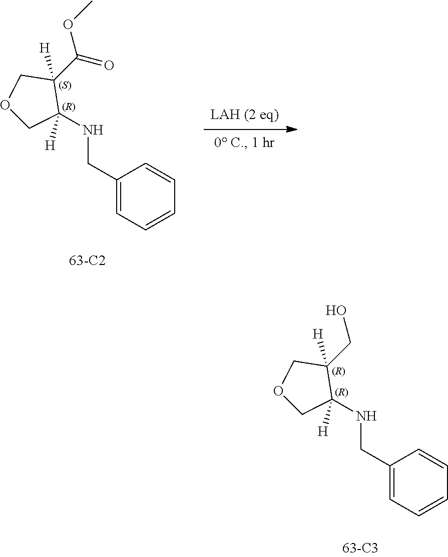

To a solution of methyl compound 63-C2 (2.0 g, 8.50 mmol) in THF (40 mL) was added LiAlH₄ (2.5 M, 6.80 mL) at 0° C. The mixture was stirred at 0° C. for 1 hr. LCMS showed the reaction was completed. The reaction mixture was added water (0.65 mL) drop-wise at 0° C., then the mixture was successively added 15% NaOH (0.65 mL) and water (1.95 mL) drop-wise at 0° C. The reaction mixture was dried over Na₂SO₄, filtered, and concentrated under reduced pressure to give compound 63-C3 (1.7 g, 96.49% yield) as a yellow oil.

¹H NMR: (400 MHz, DMSO-d₆)

δ 7.27-7.36 (m, 4H) 7.18-7.26 (m, 1H) 4.60 (br s, 1H) 3.67-3.76 (m, 4H) 3.57-3.67 (m, 2H) 3.46 (dd, J=8.38, 5.63 Hz, 2H) 3.26 (q, J=5.92 Hz, 1H) 2.24-2.34 (m, 1H) 2.19 (br d, J=2.25 Hz, 1H)

### General Procedure for Preparation of Compound 63-C4

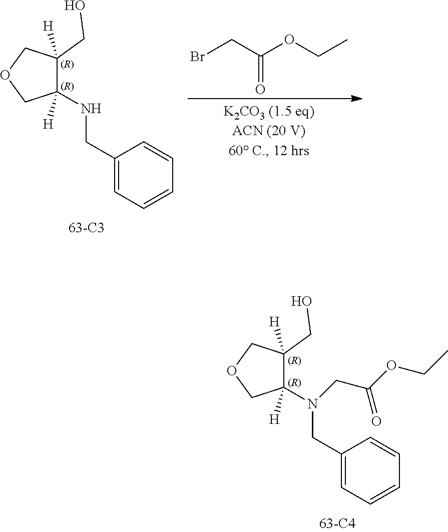

To a solution of compound 63-C3 (1.7 g, 8.20 mmol) in MeCN (36 mL) was added K₂CO₃ (1.70 g, 12.30 mmol) and ethyl 2-bromoacetate (2.05 g, 12.30 mmol, 1.36 mL) at 20° C. The mixture was stirred at 60° C. for 12 hrs. TLC showed the reaction was completed. The reaction mixture was then diluted with water (30 mL), extracted with ethyl acetate (3×50 mL), the combined organic layer was washed with brine (50 mL), dried over anhydrous Na₂SO₄, filtered, concentrated under reduced pressure to give crude product. The residue was purified by flash silica gel chromatography (ISCO®; 12 g SepaFlash® Silica Flash Column, Eluent of 24% Ethylacetate/Petroleum ethergradient @ 36 mL/min) to give compound 63-C4 (1.8 g, 74.81% yield) as a yellow oil.

¹H NMR: (400 MHz, CHLOROFORM-d)

δ 7.31-7.42 (m, 4H) 7.27-7.31 (m, 1H) 4.55 (br s, 1H) 4.06-4.22 (m, 3H) 3.85-3.99 (m, 3H) 3.71-3.81 (m, 3H) 3.67 (dd, J=8.76, 6.75 Hz, 1H) 3.58 (ddd, J=7.60, 5.47, 4.19 Hz, 1H) 3.20-3.37 (m, 2H) 2.68-2.80 (m, 1H) 1.24 (t, J=7.19 Hz, 3H)

### General Procedure for Preparation of Compound 63-C5A;

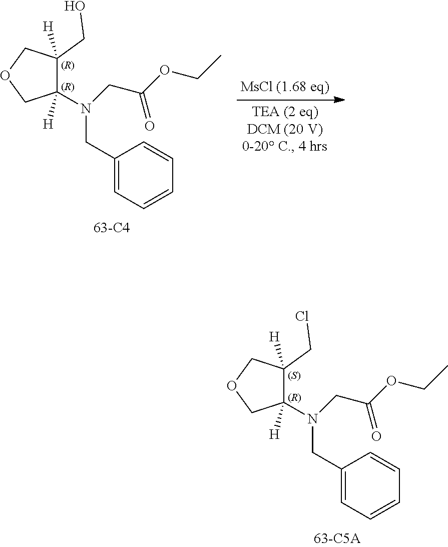

To a solution of compound 63-C4 (1.8 g, 6.14 mmol) and TEA (1.24 g, 12.27 mmol, 1.71 mL) in dichloromethane (35 mL) was added dropwise a solution of MsCl (1.18 g, 10.30 mmol, 797.30 μL) in dichloromethane (5 mL) at 0° C. under N₂. The mixture was stirred at 20° C. for 4 hrs. LCMS showed the reaction was completed. The reaction mixture was poured into ice water (40 mL), extracted with dichloromethane (3×40 mL), the combined organic layer was washed with brine (50 mL), dried over anhydrous Na₂SO₄, filtered, concentrated under reduced pressure to give residue. The residue was purified by flash silica gel chromatography (ISCO®; X g SepaFlash® Silica Flash Column, Eluent of 0˜20% Ethyl acetate/Petroleum ethergradient @ 150 mL/min) to give compound 63-C5A (1.1 g, 57.50% yield) as a colorless oil.

¹H NMR: (400 MHz, CHLOROFORM-d)

δ 7.27-7.39 (m, 5H) 4.14 (q, J=7.13 Hz, 2H) 3.78-4.06 (m, 8H) 3.60 (t, J=11.01 Hz, 1H) 3.16-3.37 (m, 2H) 2.60-2.73 (m, 1H) 1.26 (t, J=7.19 Hz, 3H)

### General Procedure for Preparation of Compound 63-C7

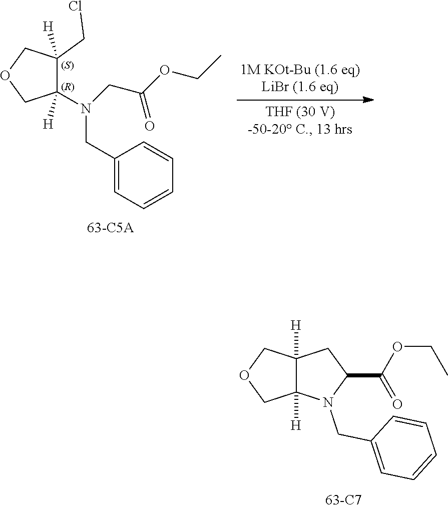

To a solution of compound 63-C5A (500 mg, 1.60 mmol) and LiBr (222.83 mg, 2.57 mmol, 64.40 μL) in THF (15 mL) was stirred at 20° C. for 1 hr. Then t-BuOK (1 M, 2.57 mL) was added to the solution at −50° C. The mixture was stirred at 20° C. for 12 hrs. TLC (ethyl acetate/petroleum ether=1/3) showed half of starting material was remained and new spot was generated. The reaction mixture was quenched with aqueous ammonium chloride solution (15 mL) at 0° C. and extracted with ethyl acetate (3×15 mL), the combined organic layer was washed with brine (15 mL), dried over anhydrous Na₂SO₄, filtered, concentrated under reduced pressure to give crude product. The residue was purified by prep-TLC (SiO₂, Petroleum ether: Ethyl acetate=4:1) to give compound 63-C7 (100 mg, 21.52% yield) as a yellow oil.

¹H NMR: ET73387-24-P1C (400 MHz, DMSO-d₆)

δ 7.21-7.39 (m, 5H) 3.94-4.09 (m, 2H) 3.87 (d, J=12.92 Hz, 1H) 3.44-3.59 (m, 2H) 3.33-3.39 (m, 2H) 3.29 (dd, J=8.97, 4.33 Hz, 1H) 3.16 (d, J=9.29 Hz, 1H) 2.99 (dd, J=9.22, 4.83 Hz, 1H) 2.69-2.84 (m, 1H) 2.16-2.29 (m, 1H) 1.58 (td, J=11.20, 8.60 Hz, 1H) 1.17 (t, J=7.09 Hz, 3H)

### General Procedure for Preparation of Compound 63-C8

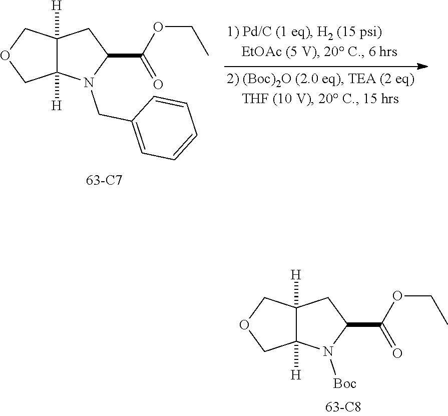 [SMILES: CCOC(=O)[C@@H]1C[C@@H]2COC[C@@H]2N1Cc1ccccc1 ; CCOC(=O)[C@@H]1C[C@@H]2COC[C@@H]2N1C(=O)OC(C)(C)C]

To a solution of compound 63-C7 (100 mg, 308.71 μmol) in EtOAc (5 mL) was added Pd/C (328.53 mg, 308.71 μmol, 10% purity) at 20° C. The mixture was stirred at 20° C. for 6 hrs under H₂ (15 psi). LCMS showed the reaction was completed. The reaction mixture was filtrated and the filtrate was concentrated under reduced pressure to give a residue. The residue was dissolved in THF (5 mL). Then TEA (62.48 mg, 617.41 μmol, 85.94 μL) and (Boc) 20 (134.75 mg, 617.41 μmol, 141.84 μL) were added to the solution. The reaction mixture was stirred at 20° C. for 15 hrs. LCMS showed the starting material was consumed, desired product Mass was detected. The reaction mixture was concentrated under reduced pressure to give crude product. The crude product was purified by prep-TLC (SiO₂, Petroleum ether: Ethyl acetate=3:1) to give compound 63-C8 (100 mg, 96.50% yield) as a colorless oil.

¹H NMR: (400 MHz, CHLOROFORM-d)

δ 4.35-4.57 (m, 2H) 4.14-4.26 (m, 2H) 3.96-4.12 (m, 1H) 3.67-3.89 (m, 2H) 3.51-3.64 (m, 1H) 2.83-3.00 (m, 1H) 2.44 (ddt, J=18.65, 13.46, 9.27, 9.27 Hz, 1H) 1.98-2.14 (m, 1H) 1.46 (d, J=17.13 Hz, 9H) 1.24-1.33 (m, 3H)

### General Procedure for Preparation of Compound 63-A9

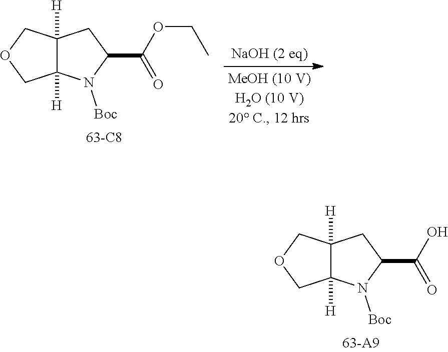 [SMILES: CCOC(=O)[C@@H]1C[C@@H]2COC[C@@H]2N1C(=O)OC(C)(C)C ; CC(C)(C)OC(=O)N1[C@H](C(=O)O)C[C@@H]2COC[C@@H]21]

To a solution of compound 63-C8 (100 mg, 297.90 μmol) in MeOH (1 mL) was added the solution of NaOH (23.83 mg, 595.79 μmol) in H₂O (1 mL) at 20° C. The mixture was stirred at 20° C. for 12 hrs. LCMS showed the starting material was consumed and desired product Mass was detected. The reaction mixture was concentrated under reduced pressure to give the residue. The residue was dissolved in water (5 mL) and adjusted to pH=4-5 with 1 M HCl and extracted with CH₂Cl₂ (3×15 mL), the combined organic layer was washed with brine (15 mL), dried over anhydrous Na₂SO₄, filtered, concentrated under reduced pressure to give compound 63-A9 (80 mg, 73.07% yield) as a yellow oil.

¹H NMR: (400 MHz, CHLOROFORM-d)

δ 4.26-4.60 (m, 2H) 3.89-4.25 (m, 1H) 3.53-3.85 (m, 3H) 2.83-3.09 (m, 1H) 2.38-2.67 (m, 1H) 1.99-2.36 (m, 1H) 1.39-1.60 (m, 9H) General procedure for preparation of Compound 63-11

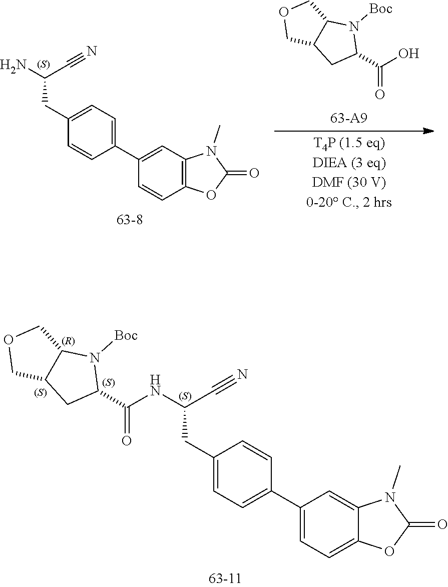

To a solution of Compound 63-8 (30.00 mg, 102.28 μmol) in DMF (1 mL) was added 63-A9 (29.24 mg, 102.28 μmol) and DIEA (39.66 mg, 306.83 μmol, 53.44 μL) at 0° C., then T₄P (110.54 mg, 153.42 μmol, 50% purity) was added to the reaction mixture. The reaction mixture was stirred at 20° C. for 2 hrs. LCMS showed the starting material was consumed and desired product Mass was detected. The reaction mixture was filtered and the filtrate was purified by prep-HPLC and lyophilized to give Compound 63-11 (20 mg, 35.98% yield) as light-yellow solid.

Column: Waters Xbridge Prep OBD C18 150*40 mm*10 um; mobile phase: [H₂O (10 mM NH₄HCO₃)-ACN]; gradient: 40%-70% B over 8.0 min

¹H NMR: (400 MHz, METHANOL-d₄)

δ 7.63 (dd, J=8.22, 3.83 Hz, 2H) 7.36-7.47 (m, 4H) 7.27-7.35 (m, 1H) 5.08-5.19 (m, 1H) 3.92-4.41 (m, 3H) 3.68 (br d, J=5.40 Hz, 1H) 3.50-3.60 (m, 1H) 3.45 (s, 3H) 3.33-3.39 (m, 1H) 3.09-3.28 (m, 2H) 2.79-2.98 (m, 1H) 2.29-2.55 (m, 1H) 1.51-1.81 (m, 1H) 1.33-1.50 (m, 9H)

### General Procedure for Preparation of Compound 187: (2S,3aS,6aR)—N—((S)-1-cyano-2-(4-(3-methyl-2-oxo-2,3-dihydrobenzo[d]oxazol-5-yl)phenyl)ethyl)hexahydro-1H-furo[3,4-b]pyrrole-2-carboxamide

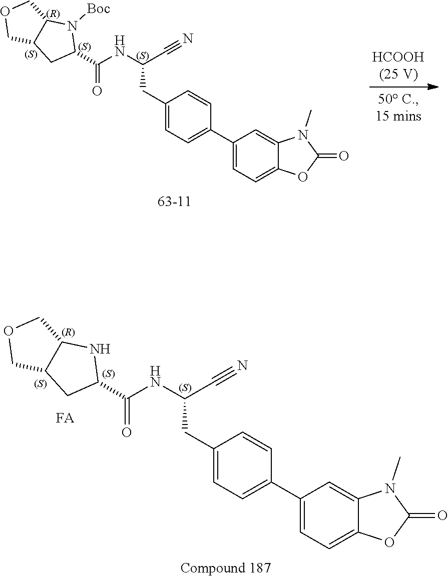

A solution of 63-11 (20 mg, 36.80 μmol) in HCOOH (0.5 mL) was stirred at 50° C. for 15 mins. LCMS showed the starting material was consumed and desired product Mass was detected. The reaction mixture was concentrated under reduced pressure to give crude product. The crude product was lyophilized to give cis mixture compound 187: (2S,3aS,6aR)—N—((S)-1-cyano-2-(4-(3-methyl-2-oxo-2,3-dihydrobenzo[d]oxazol-5-yl)phenyl)ethyl)hexahydro-1H-furo[3,4-b]pyrrole-2-carboxamide (11.3 mg, 70.01% yield, 98.6% purity, FA salt) as off-white solid.

Note: Since the specific rotation of starting material (Cpd_63-C1) was only −0.20, the starting material was a cis mixture, which lead to the acids 63-A9 was cis mixture (The structure was confirmed by 2D NMR). There were two sets of signals in HNMR, the final target was cis mixture and the cis structure was confirmed by NOE.

¹H NMR: (400 MHz, METHANOL-d₄)

δ ppm 7.61-7.68 (m, 2H) 7.37-7.45 (m, 4H) 7.29-7.34 (m, 1H) 5.08-5.27 (m, 1H) 4.19-4.35 (m, 3H) 3.53-3.64 (m, 2H) 3.47-3.52 (m, 1H) 3.46 (s, 3H) 3.20 (br s, 3H) 2.53-2.80 (m, 1H) 1.47-1.91 (m, 1H)

### Example 64. Synthesis of Compound 183: (2S,3aS,6aR)—N—((S)-1-cyano-2-(4′-cyano-[1,1′-biphenyl]-4-yl)ethyl)hexahydro-1H-furo[3,4-b]pyrrole-2-carboxamide

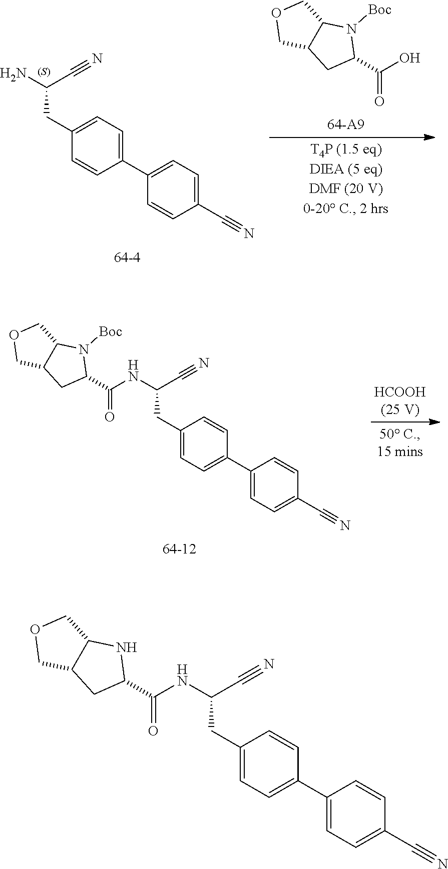

### General Procedure for Preparation of Compound 64-12;

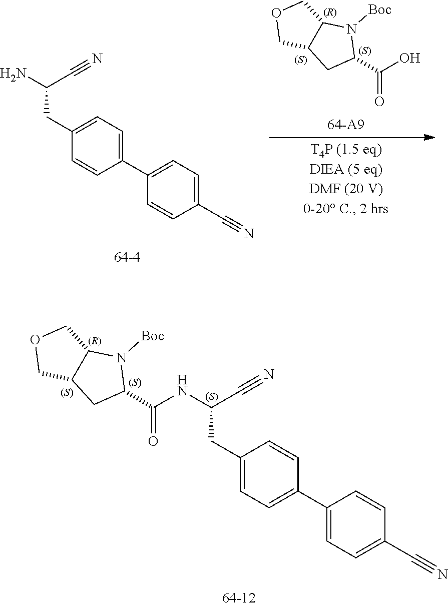

To a solution of Compound 64-4 (15.00 mg, 60.66 μmol) in DMF (0.3 mL) was added 64-A9 (17.34 mg, 60.66 μmol) and DIEA (39.20 mg, 303.28 μmol, 52.83 μL) at 0° C., then T₄P (65.56 mg, 90.98 μmol, 50% purity) was added to the reaction mixture. The reaction mixture was stirred at 0° C. for 2 hrs. LCMS showed the starting material was consumed and desired product Mass was detected. The crude product was purified by prep-HPLC and lyophilized to give Compound 12 (10 mg, 33.65% yield) as a white solid.

Column: Waters Xbridge Prep OBD C18 150*40 mm*10 um; mobile phase: [H₂O (10 mM NH₄HCO₃)-ACN]; gradient: 30%-60% B over 8.0 min

### General Procedure for Preparation of (2S,3aS,6aR)—N—((S)-1-cyano-2-(4′-cyano-[1,1′-biphenyl]-4-yl)ethyl)hexahydro-1H-furo[3,4-b]pyrrole-2-carboxamide

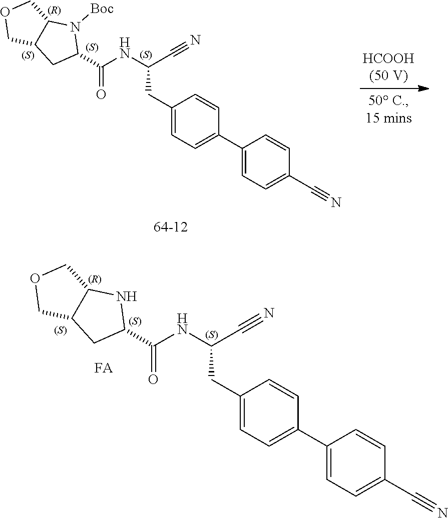

A solution of Compound 64-12 (10 mg, 20.41 μmol) in HCOOH (0.5 mL) was stirred at 50° C. for 15 mins. LCMS showed the starting material was consumed and major desired product Mass was detected. The reaction mixture was concentrated under reduced pressure to give crude product. The crude product was lyophilized to give cis mixture (2S,3aS,6aR)—N—((S)-1-cyano-2-(4′-cyano-[1,1′-biphenyl]-4-yl)ethyl)hexahydro-1H-furo[3,4-b]pyrrole-2-carboxamide (9.7 mg, 93.9% purity, FA salt) as off-white solid.

Note: Since the acids 64-A9 was cis mixture, the final target were two sets of signals in HNMR, the final target was cis mixture and the cis structure was confirmed by NOE.

H NMR: (400 MHz, METHANOL-d₄)

δ ppm 7.83 (s, 4H) 7.68-7.75 (m, 2H) 7.44-7.51 (m, 2H) 5.10-5.29 (m, 1H) 4.21-4.37 (m, 3H) 3.48-3.64 (m, 3H) 3.11-3.31 (m, 3H) 2.52-2.82 (m, 1H) 1.52-1.91 (m, 1H)

### Example 65. Synthesis of Compound 240: N—((S)-1-cyano-2-(4-(3-methyl-2-oxo-2,3-dihydrobenzo[d]oxazol-5-yl)phenyl)ethyl)-6-oxa-2-azaspiro[3.4]octane-8-carboxamide

Compound 240 (N—((S)-1-cyano-2-(4-(3-methyl-2-oxo-2,3-dihydrobenzo[d]oxazol-5-yl)phenyl)ethyl)-6-oxa-2-azaspiro[3.4]octane-8-carboxamide) is synthesized through the process in the scheme below.

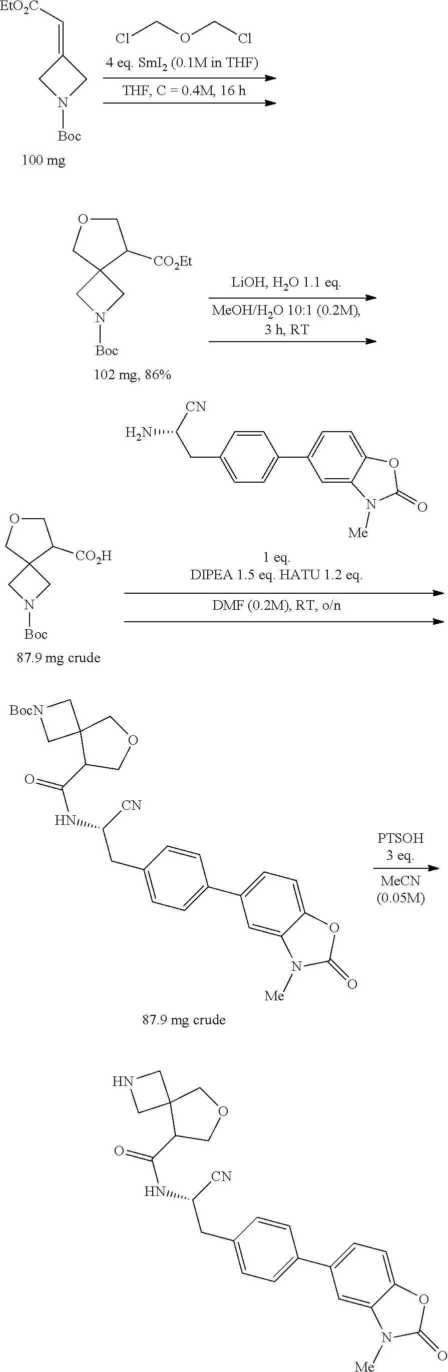

### Example 66. Synthesis of 6-(tert-butoxycarbonyl)-2-oxa-6-azaspiro[3.4]octane-8-carboxylic acid

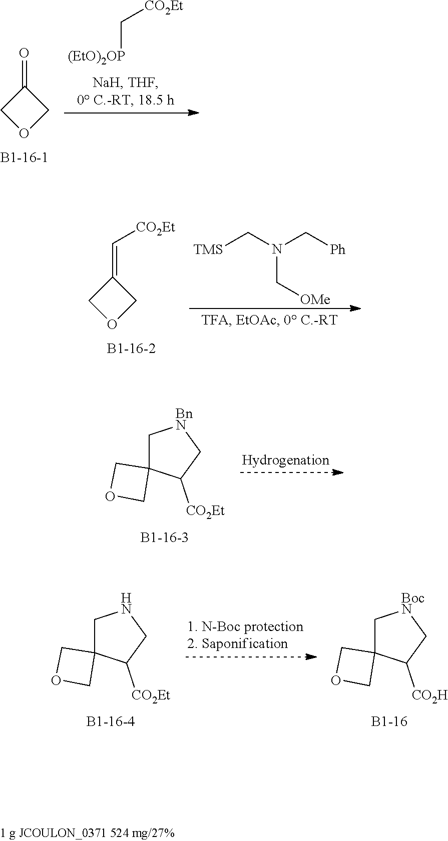

### Example 67. Synthesis of 6 aminomethanesulfonyl chloride and tert-butyl ((chlorosulfonyl)methyl)carbamate

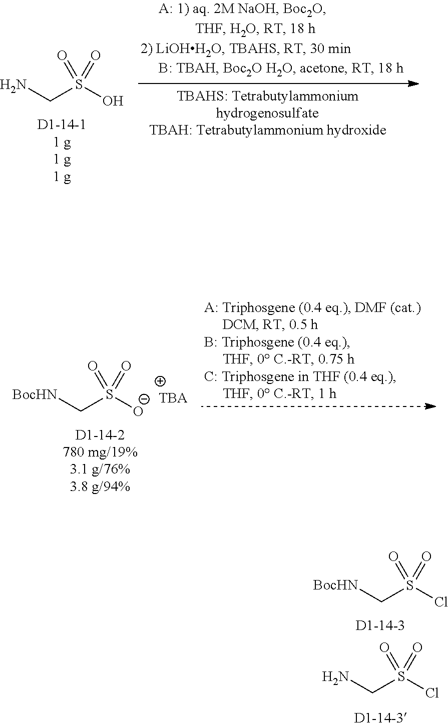

### Example 68, General Synthetic Schemes

Some of the compounds disclosed in this application are synthesized according to the synthetic schemes below.

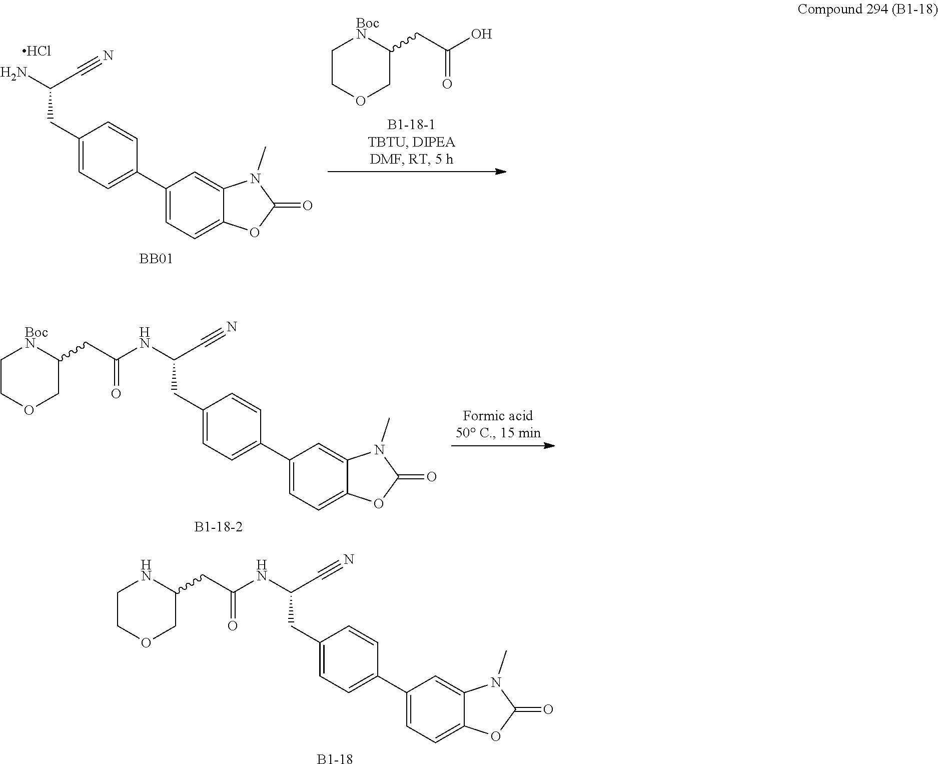

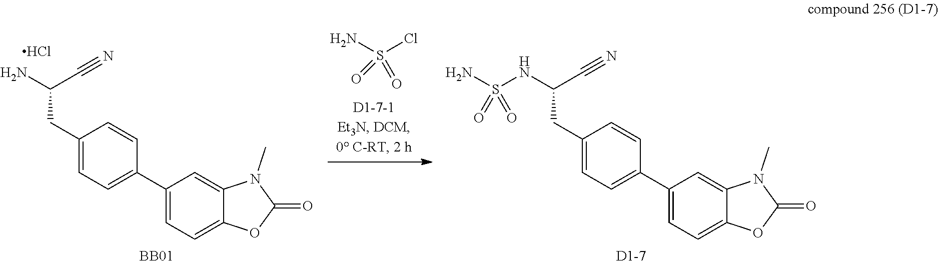 [SMILES: Cn1c(=O)oc2ccc(-c3ccc(C[C@H](N)C#N)cc3)cc21 ; Cl ; NS(=O)(=O)Cl ; Cn1c(=O)oc2ccc(-c3ccc(C[C@@H](C#N)NS(N)(=O)=O)cc3)cc21 ; "BB01" ; "D1-7"]

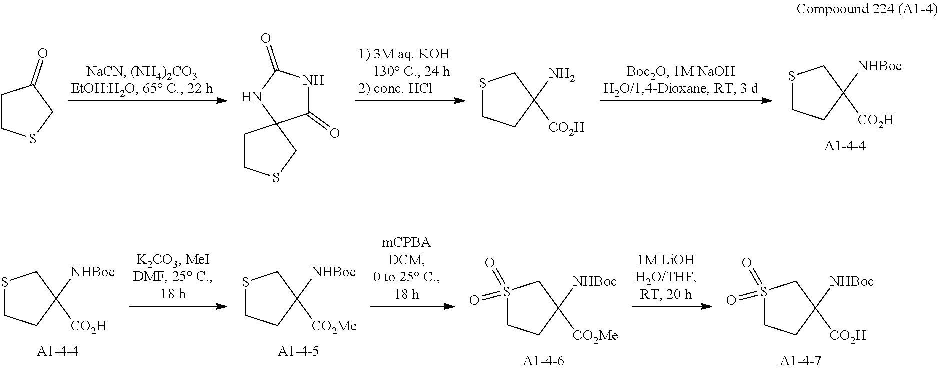

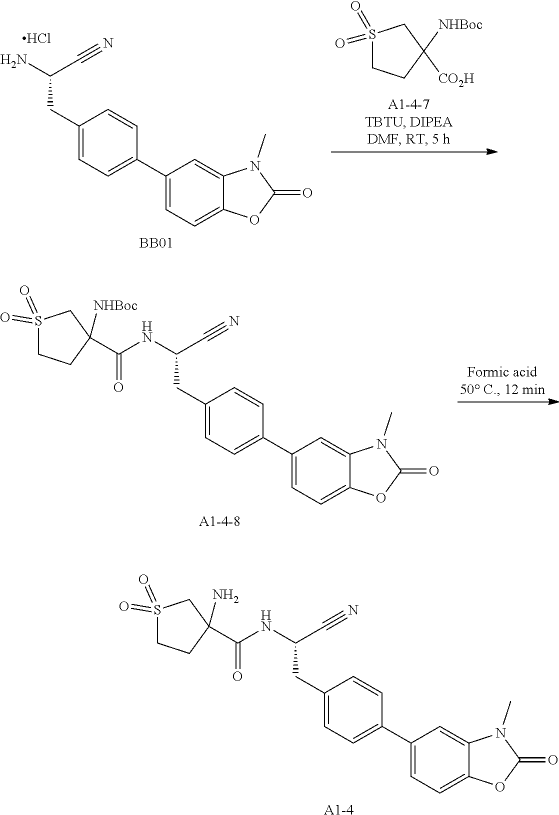

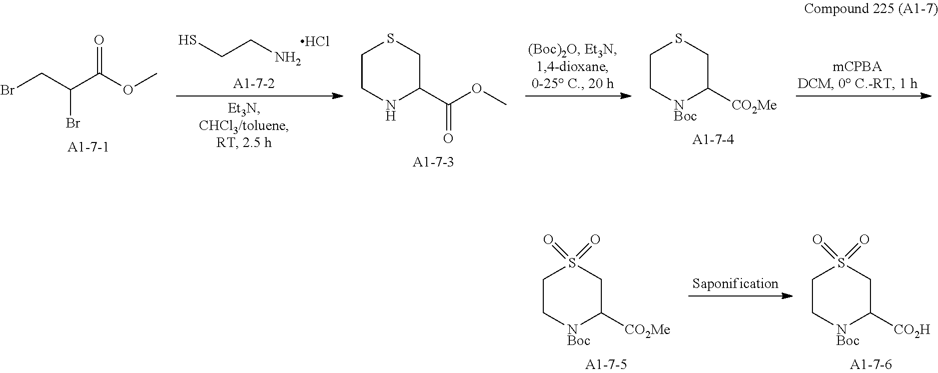

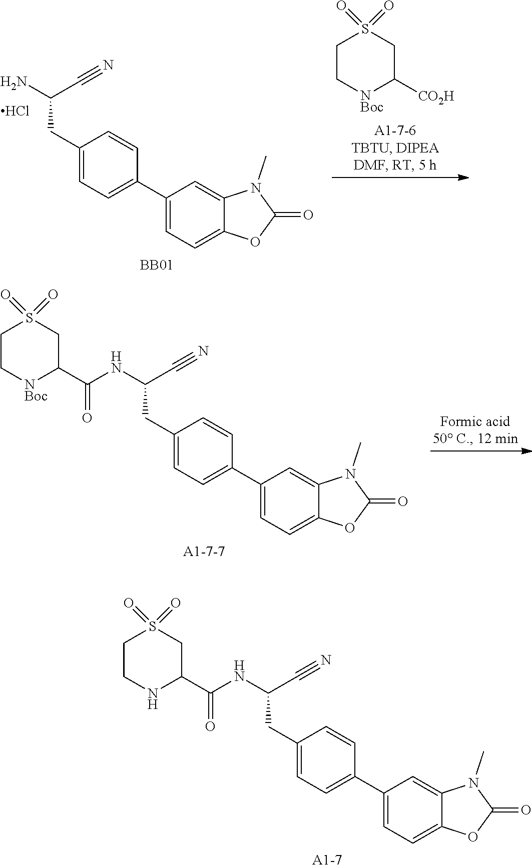

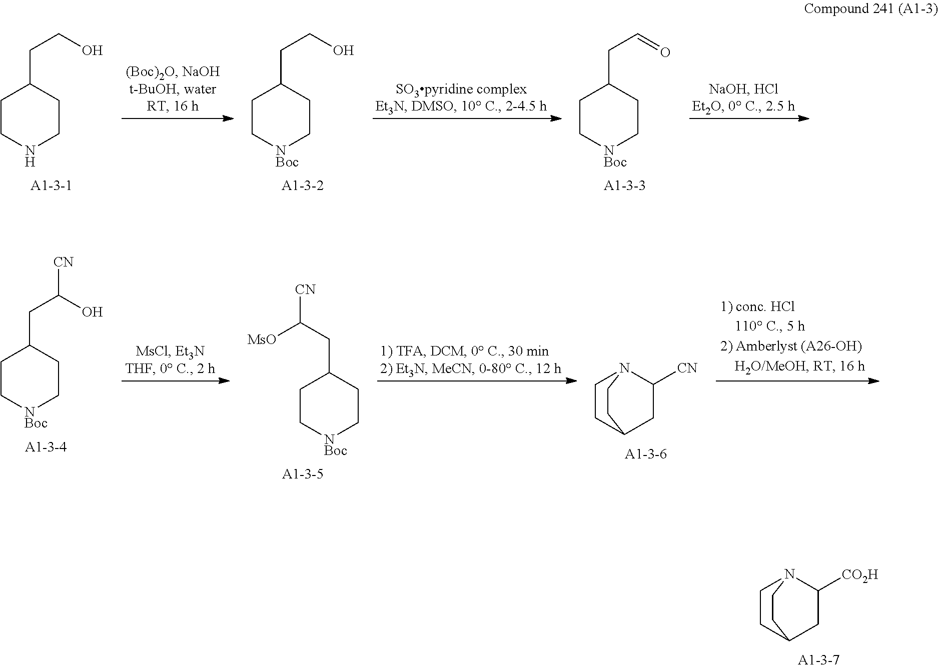

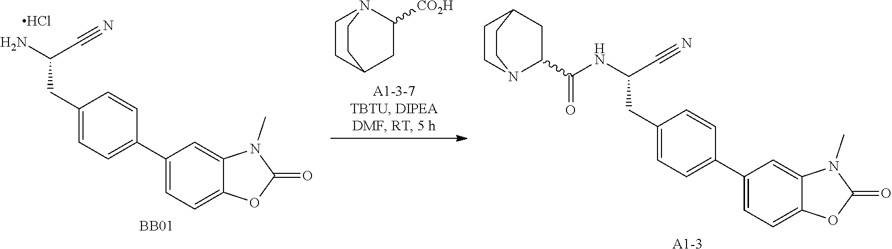 [SMILES: Cn1c(=O)oc2ccc(-c3ccc(C[C@H](N)C#N)cc3)cc21 ; Cl ; "BB01" ; O=C(O)C1CC2CCN1CC2 ; Cn1c(=O)oc2ccc(-c3ccc(C[C@@H](C#N)NC(=O)C4CC5CCN4CC5)cc3)cc21 ; "A1-3"]

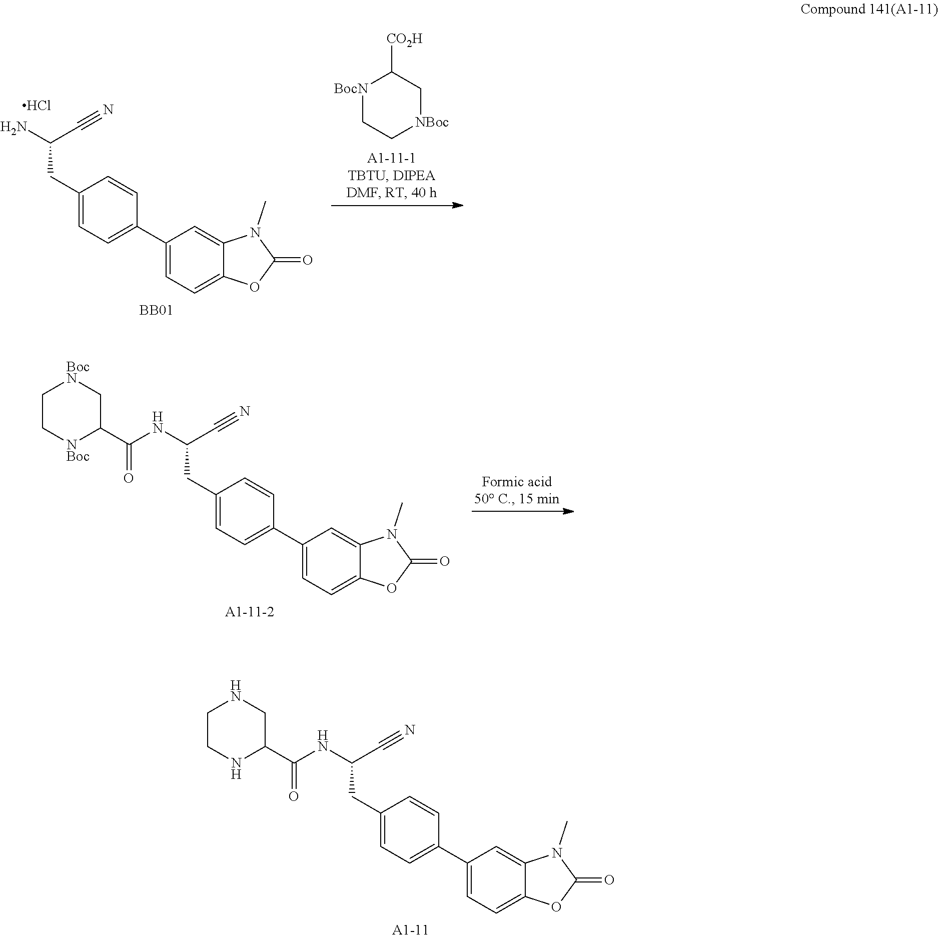

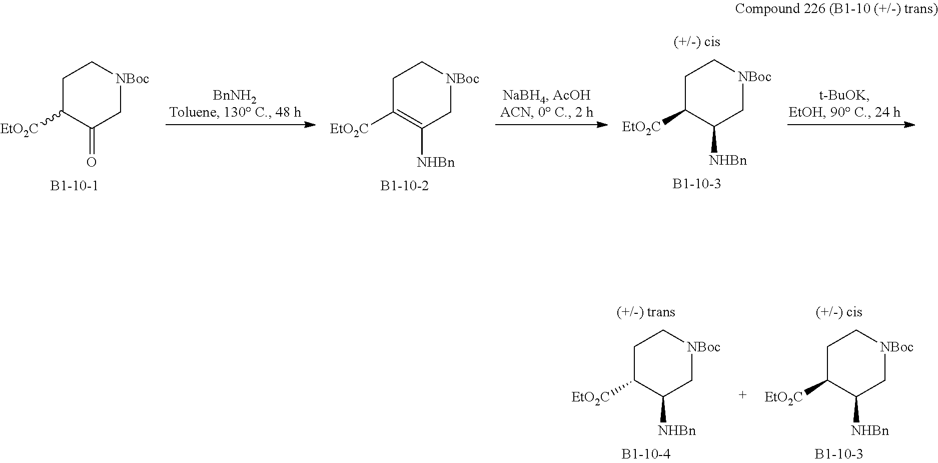

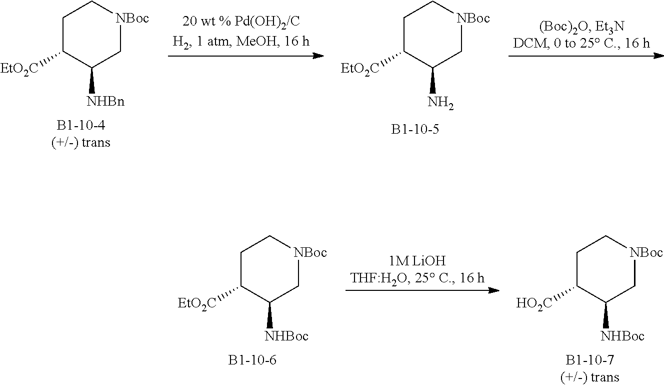

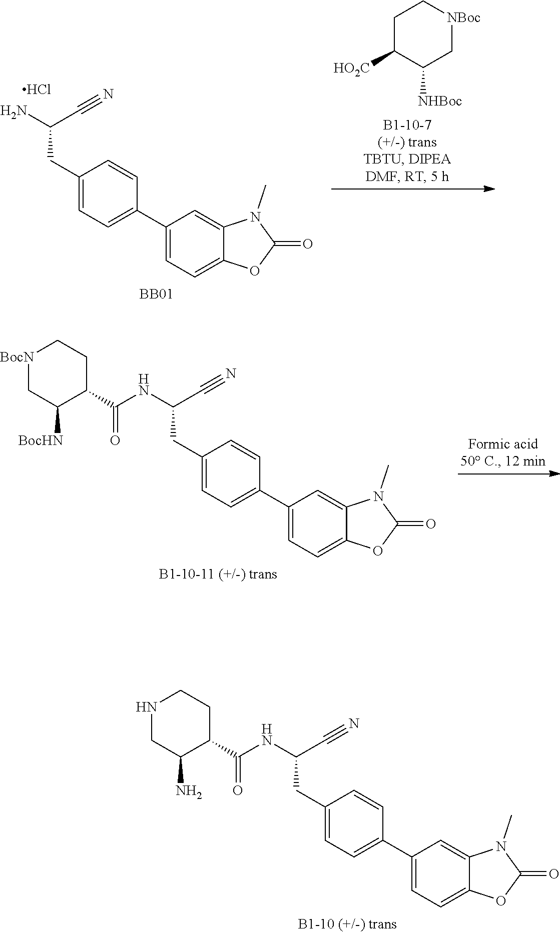

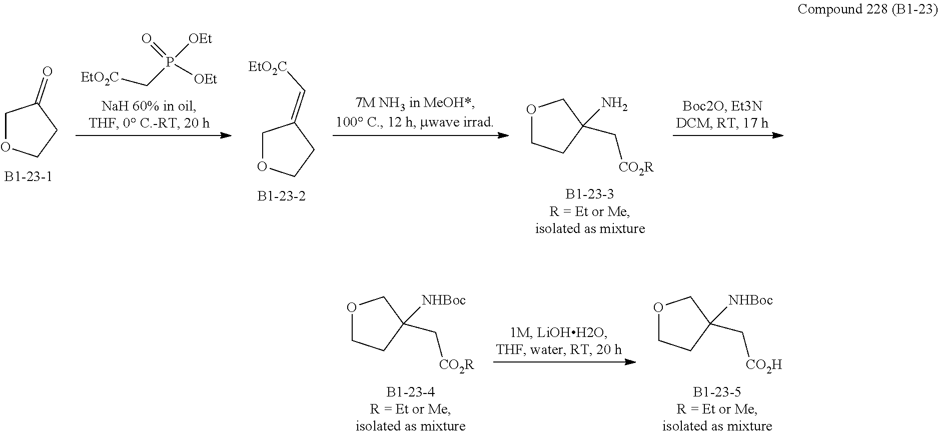

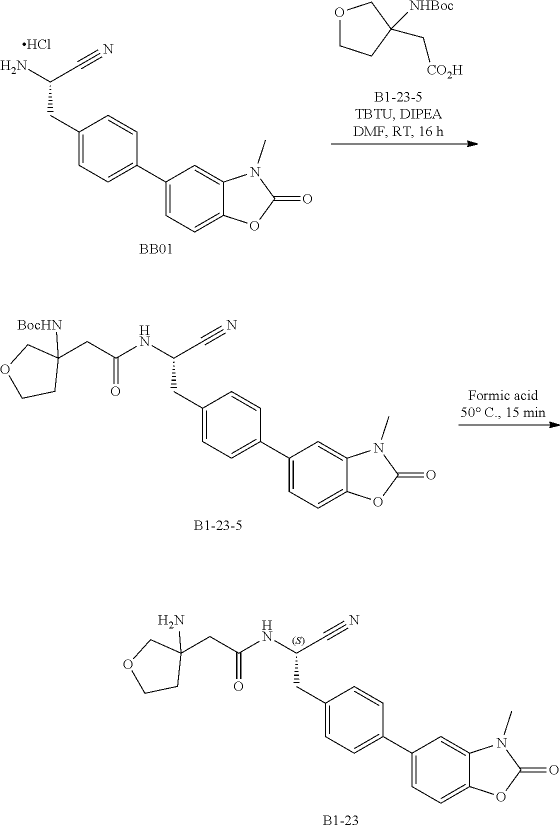

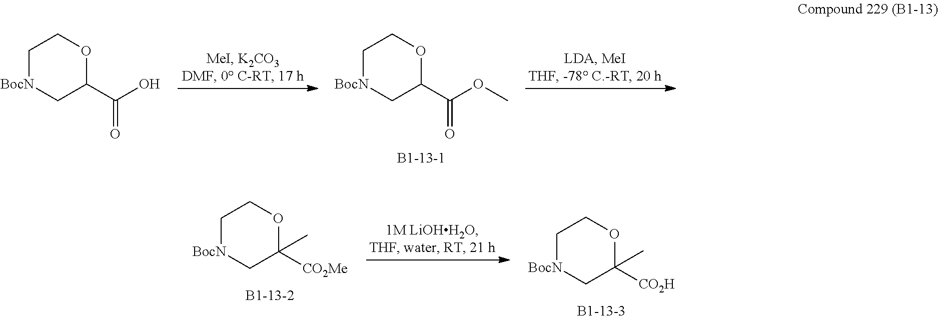 [SMILES: CC(C)(C)OC(=O)N1CCOC(C(=O)O)C1 ; COC(=O)C1CN(C(=O)OC(C)(C)C)CCO1 ; COC(=O)C1(C)CN(C(=O)OC(C)(C)C)CCO1 ; CC(C)(C)OC(=O)N1CCOC(C)(C(=O)O)C1]

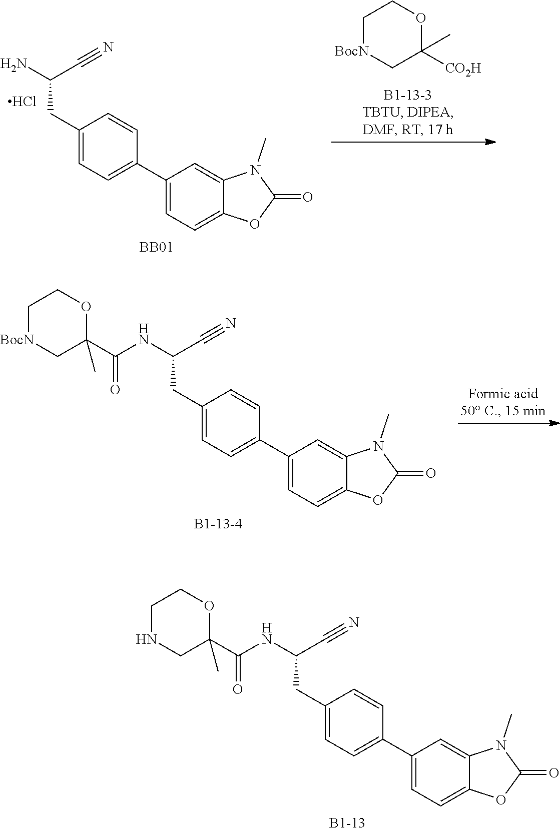

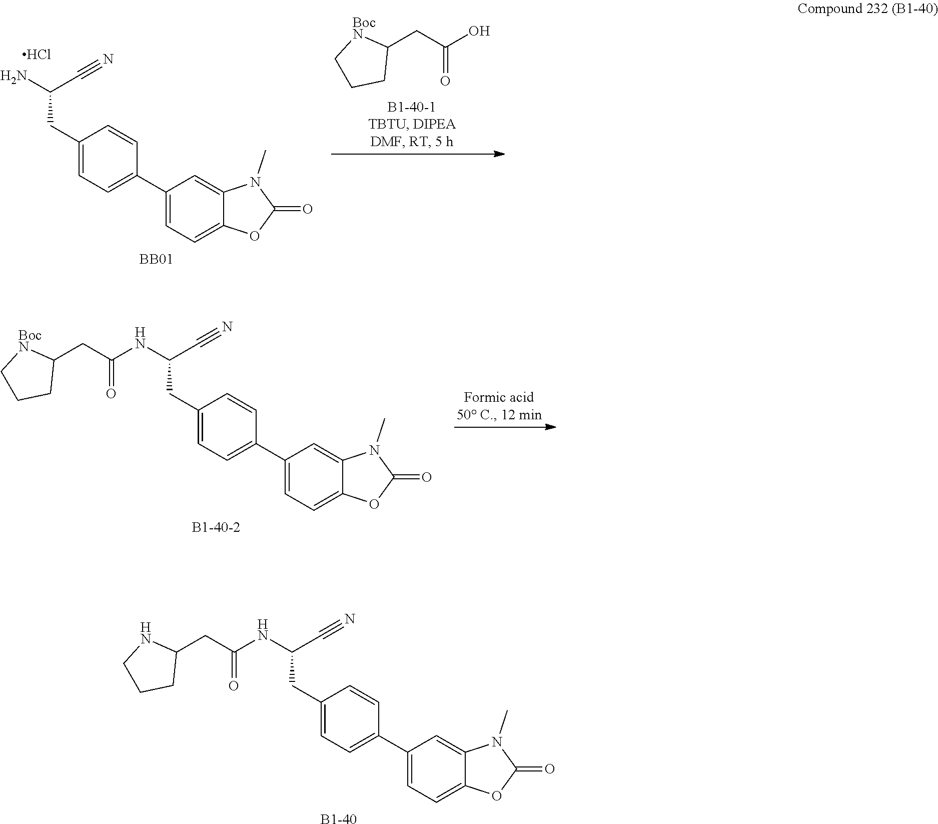

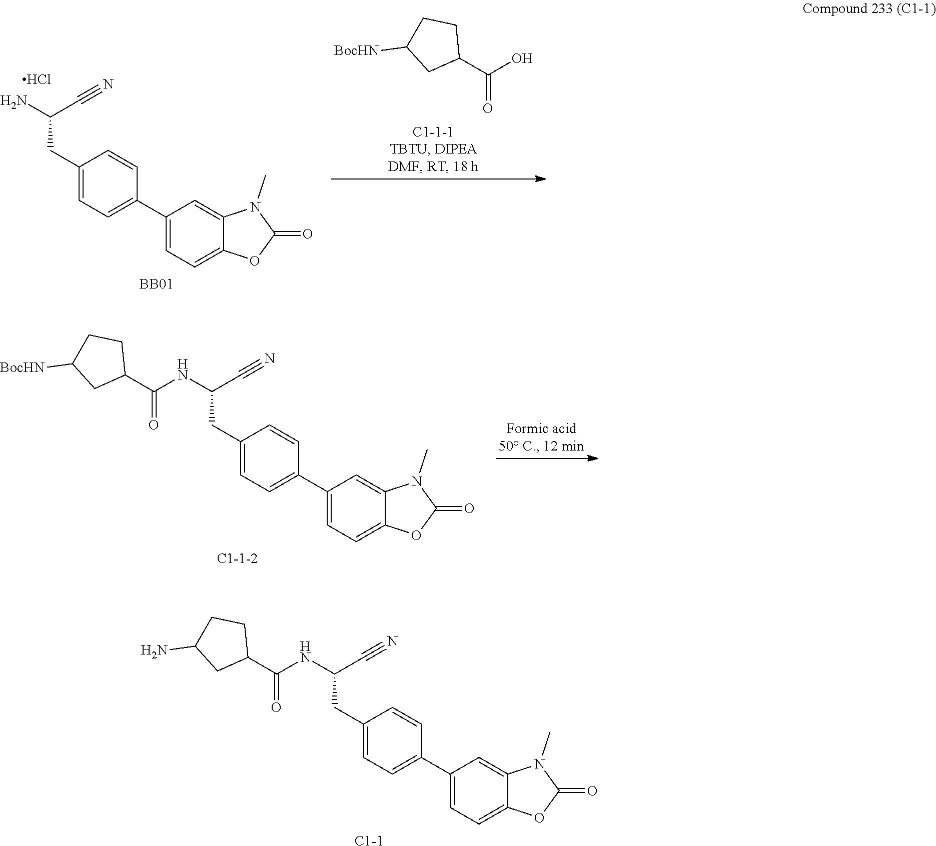

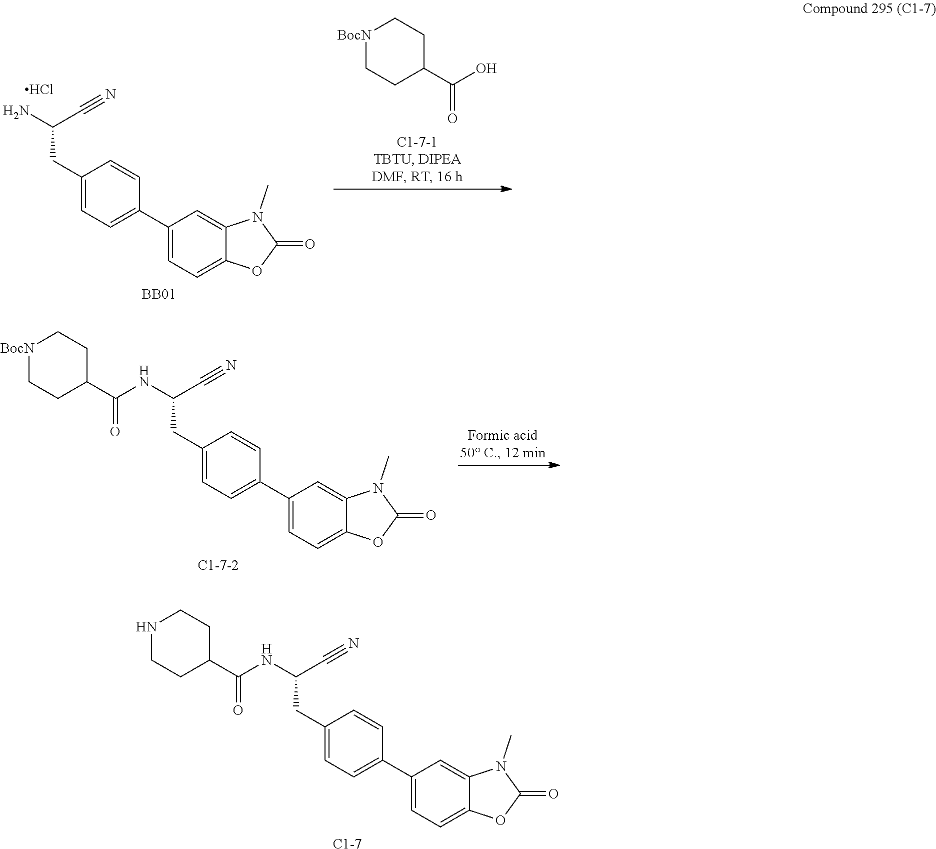

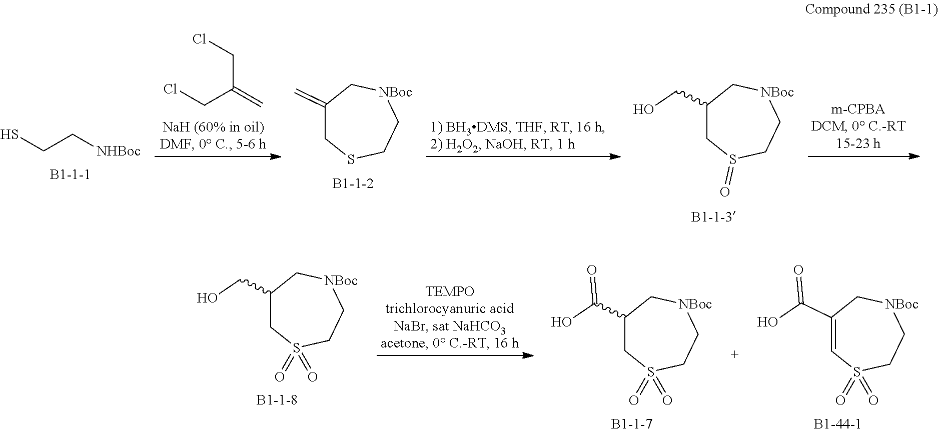

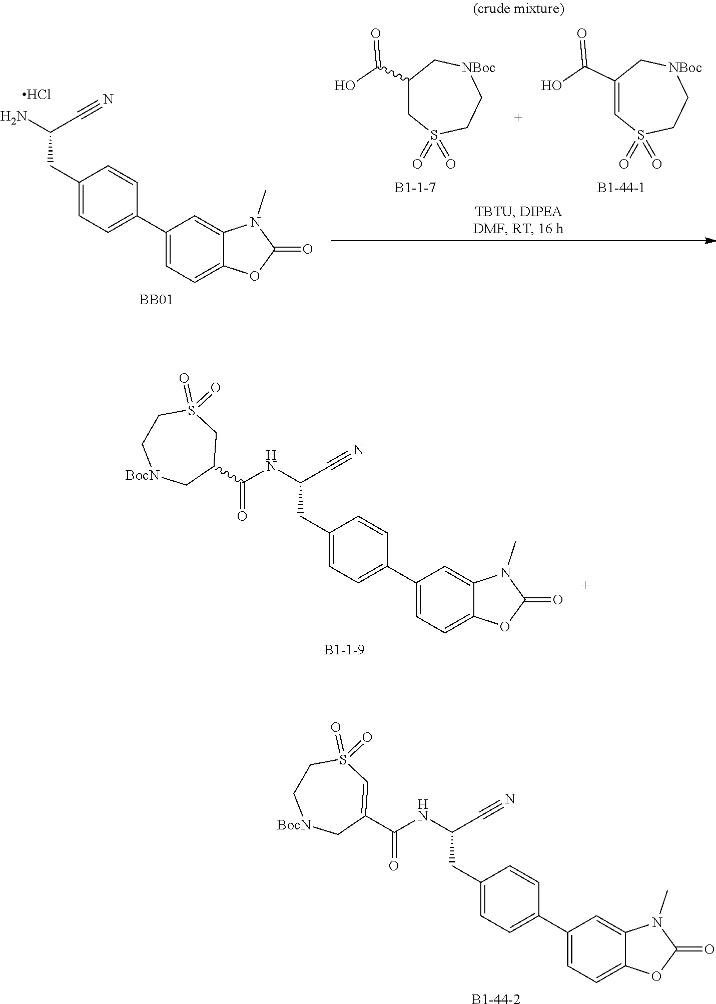

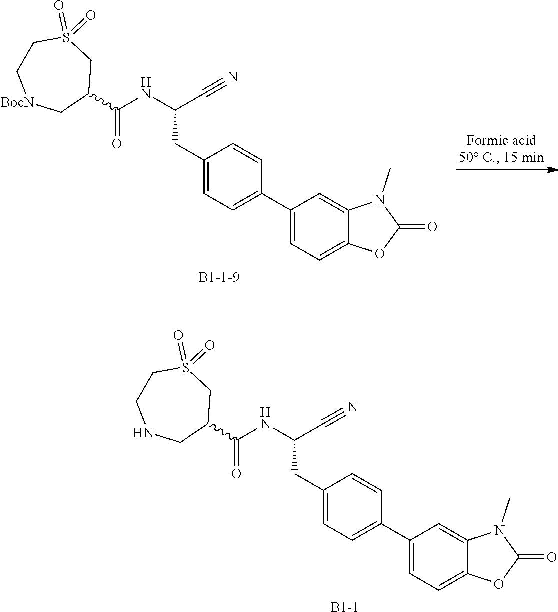 [SMILES: Cn1c(=O)oc2ccc(-c3ccc(C[C@@H](C#N)NC(=O)C4CN(C(=O)OC(C)(C)C)CCS(=O)(=O)C4)cc3)cc21 ; "B1-1-9" ; Cn1c(=O)oc2ccc(-c3ccc(C[C@@H](C#N)NC(=O)C4CNCCS(=O)(=O)C4)cc3)cc21 ; "B1-1"]

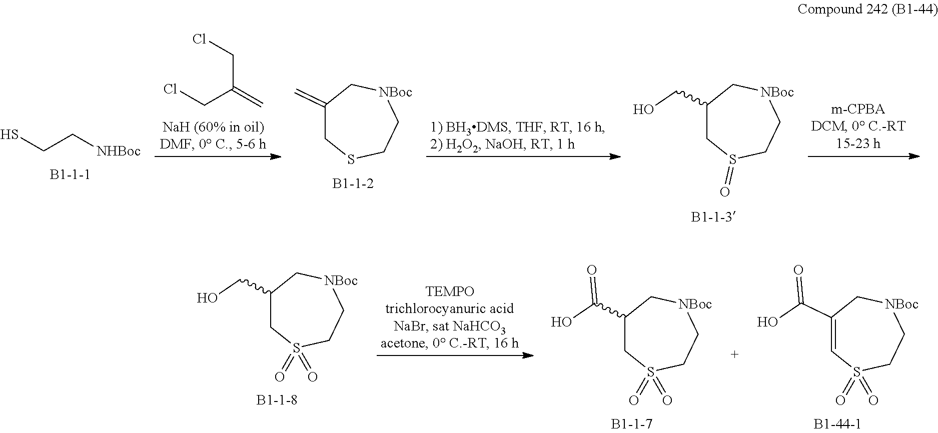

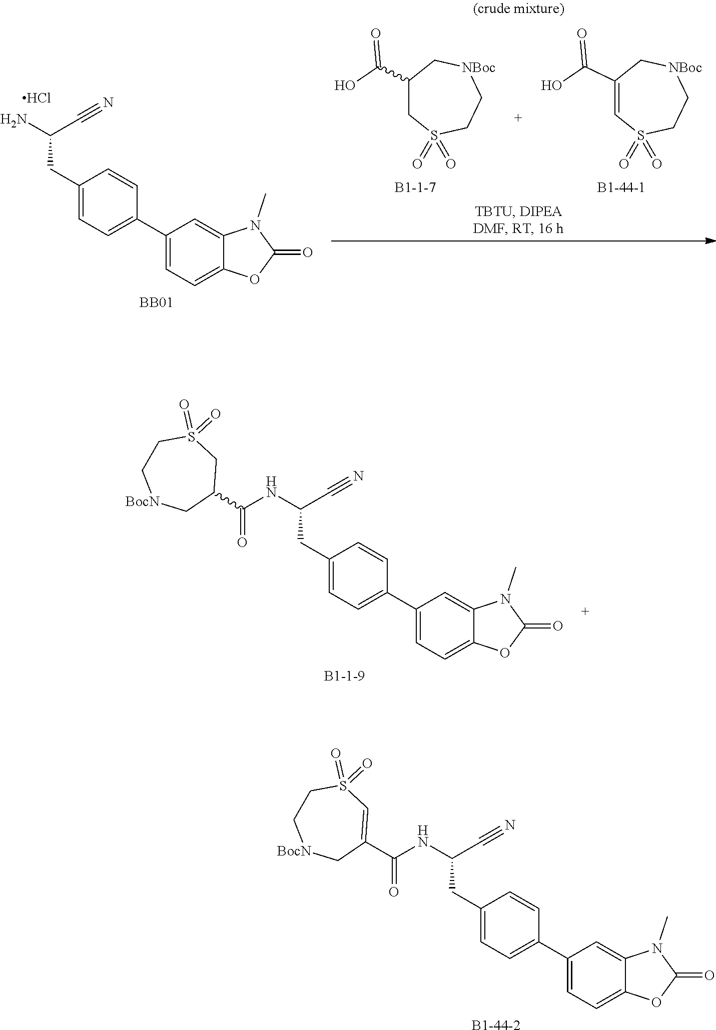

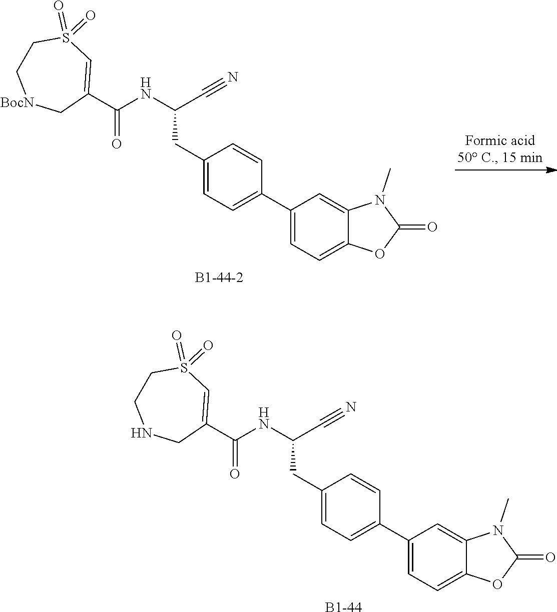 [SMILES: Cn1c(=O)oc2ccc(-c3ccc(C[C@@H](C#N)NC(=O)C4=CS(=O)(=O)CCN(C(=O)OC(C)(C)C)C4)cc3)cc21 ; "B1-44-2" ; Cn1c(=O)oc2ccc(-c3ccc(C[C@@H](C#N)NC(=O)C4=CS(=O)(=O)CCNC4)cc3)cc21 ; "B1-44"]

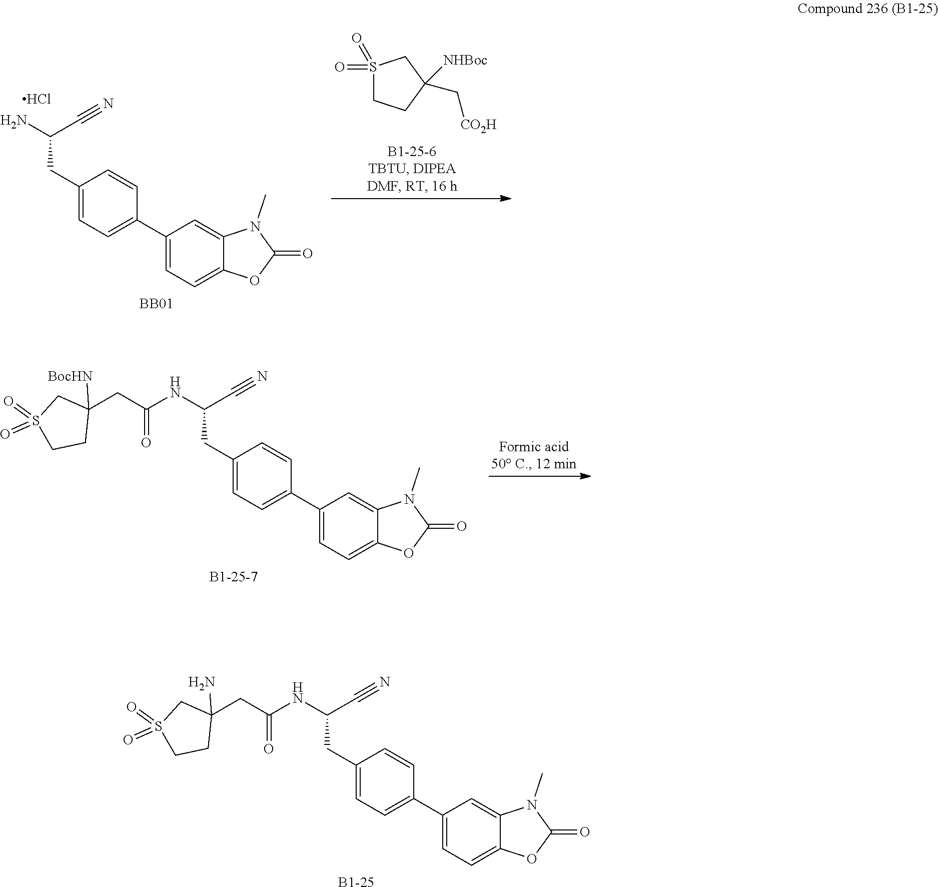

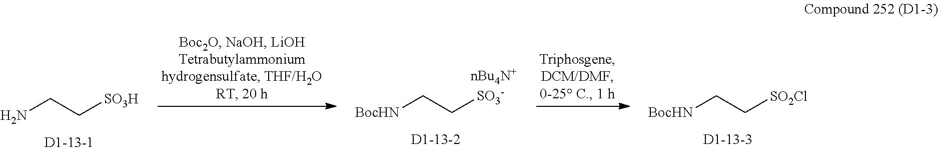

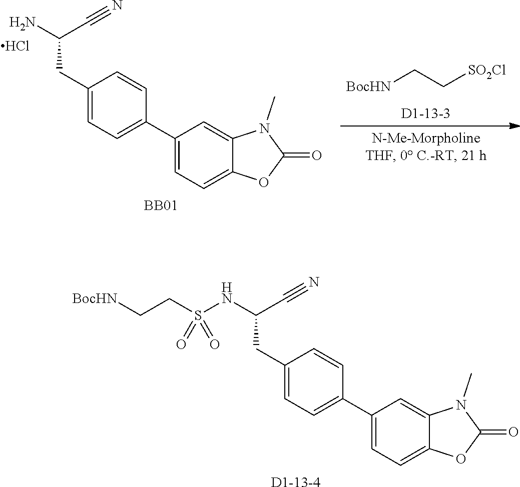 [SMILES: Cl ; Cn1c(=O)oc2ccc(-c3ccc(C[C@H](N)C#N)cc3)cc21 ; CC(C)(C)OC(=O)NCCS(=O)(=O)Cl ; "BB01" ; Cn1c(=O)oc2ccc(-c3ccc(C[C@@H](C#N)NS(=O)(=O)CCNC(=O)OC(C)(C)C)cc3)cc21 ; "D1-13-4"]

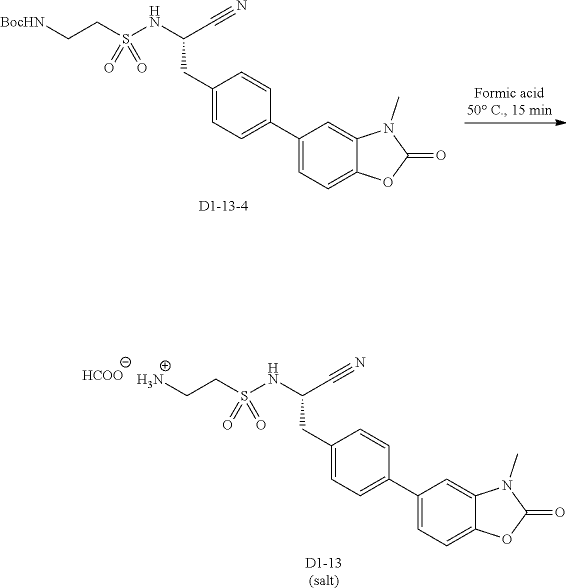 [SMILES: Cn1c(=O)oc2ccc(-c3ccc(C[C@@H](C#N)NS(=O)(=O)CCNC(=O)OC(C)(C)C)cc3)cc21 ; "D1-13-4" ; "HCOO" ; Cn1c(=O)oc2ccc(-c3ccc(C[C@@H](C#N)NS(=O)(=O)CC[NH3+])cc3)cc21 ; "D1-13(salt)"]

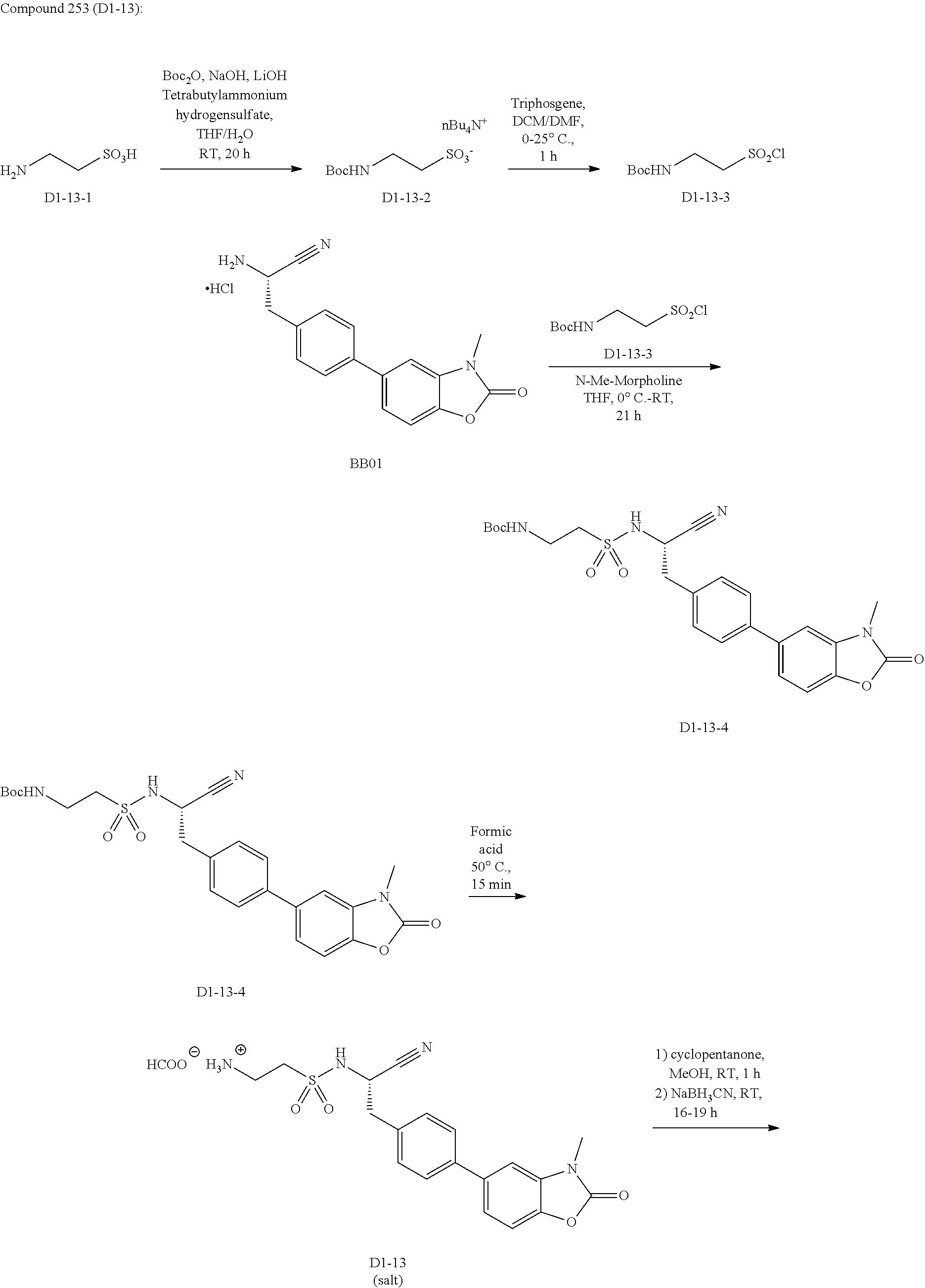

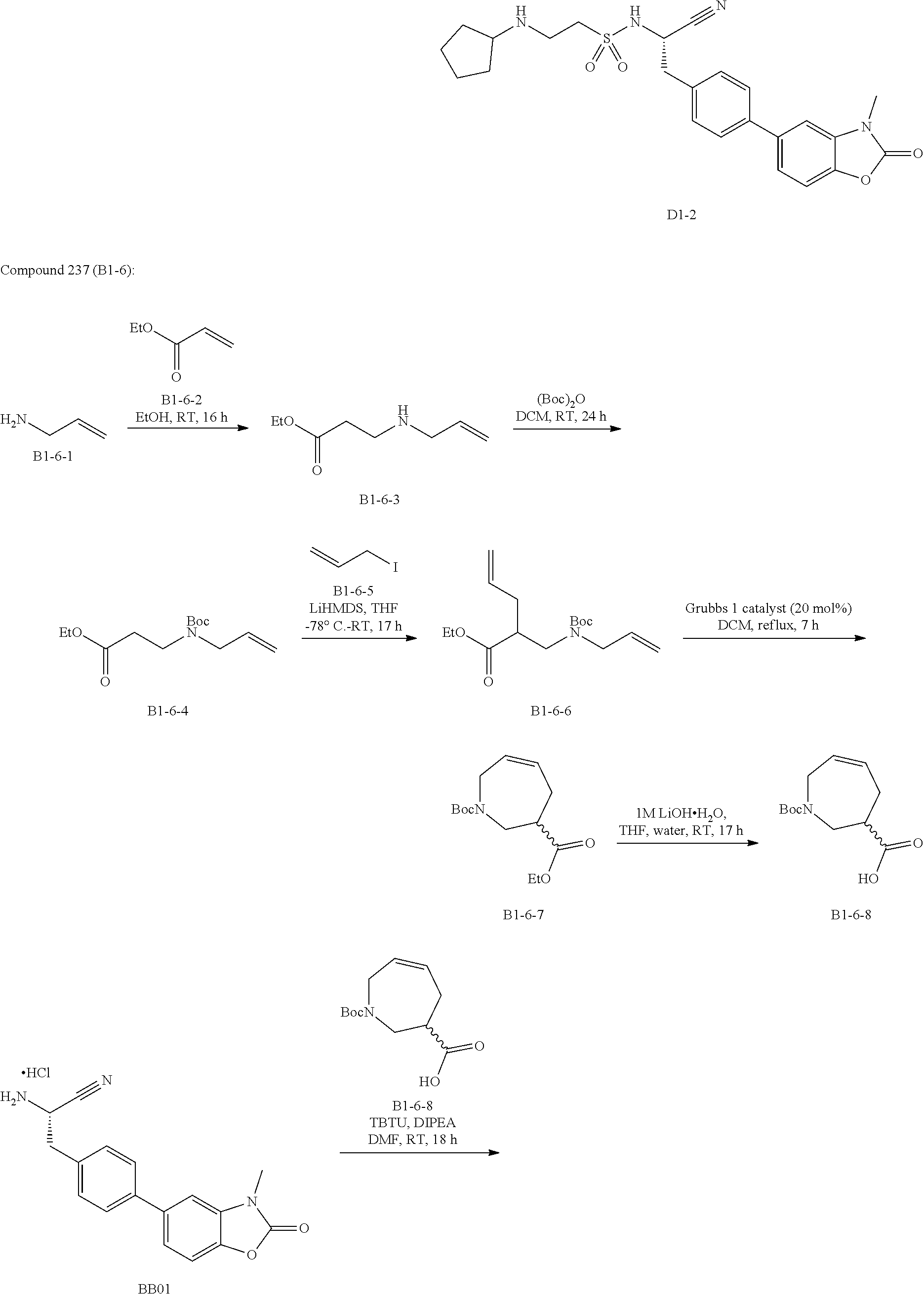

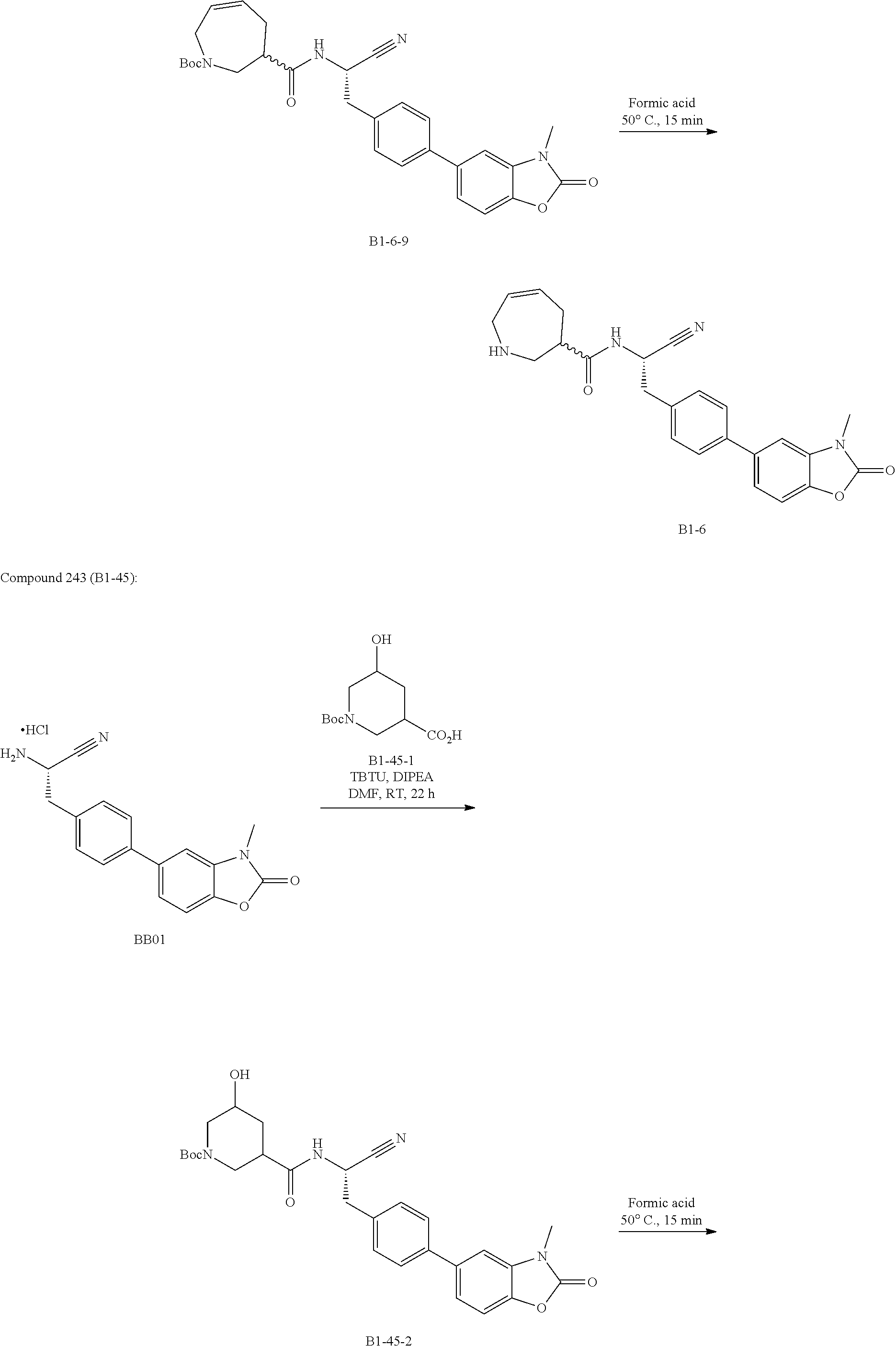

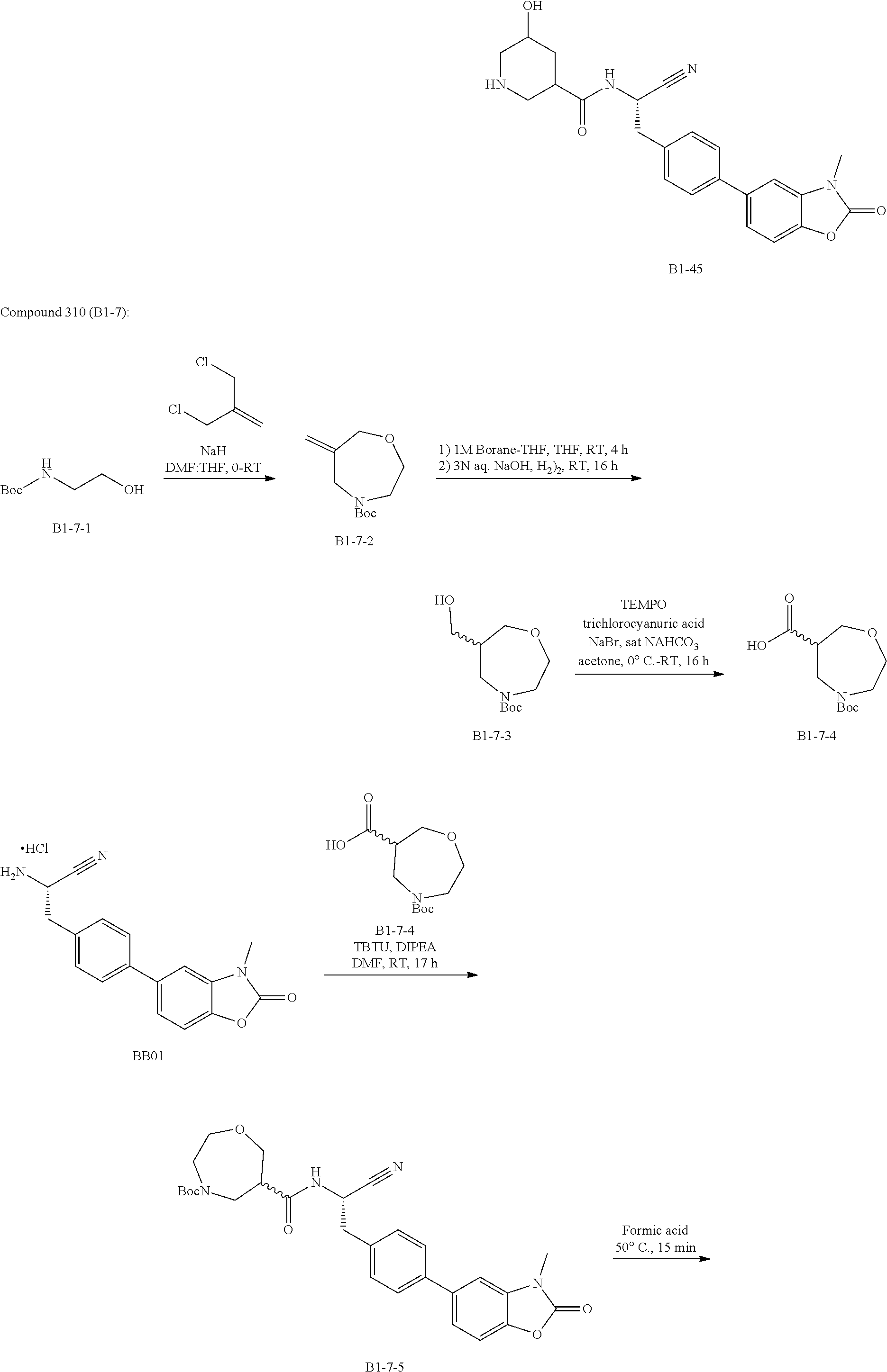

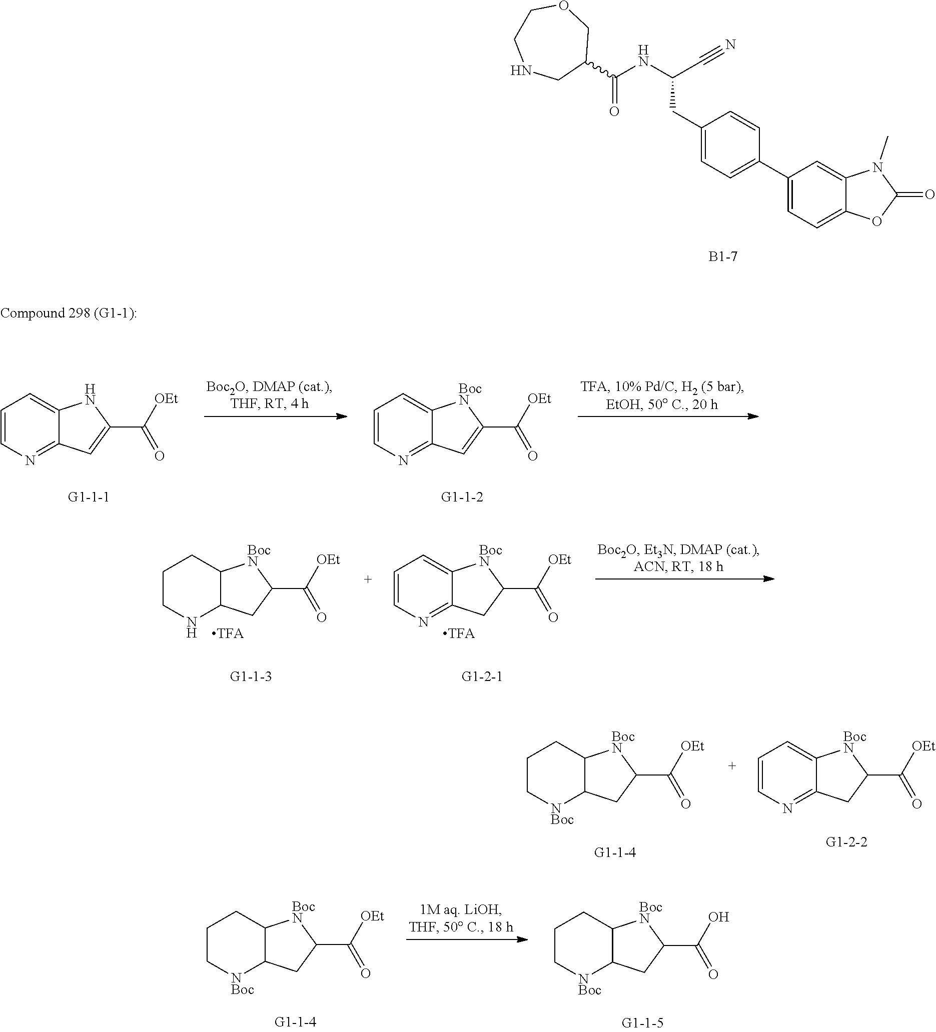

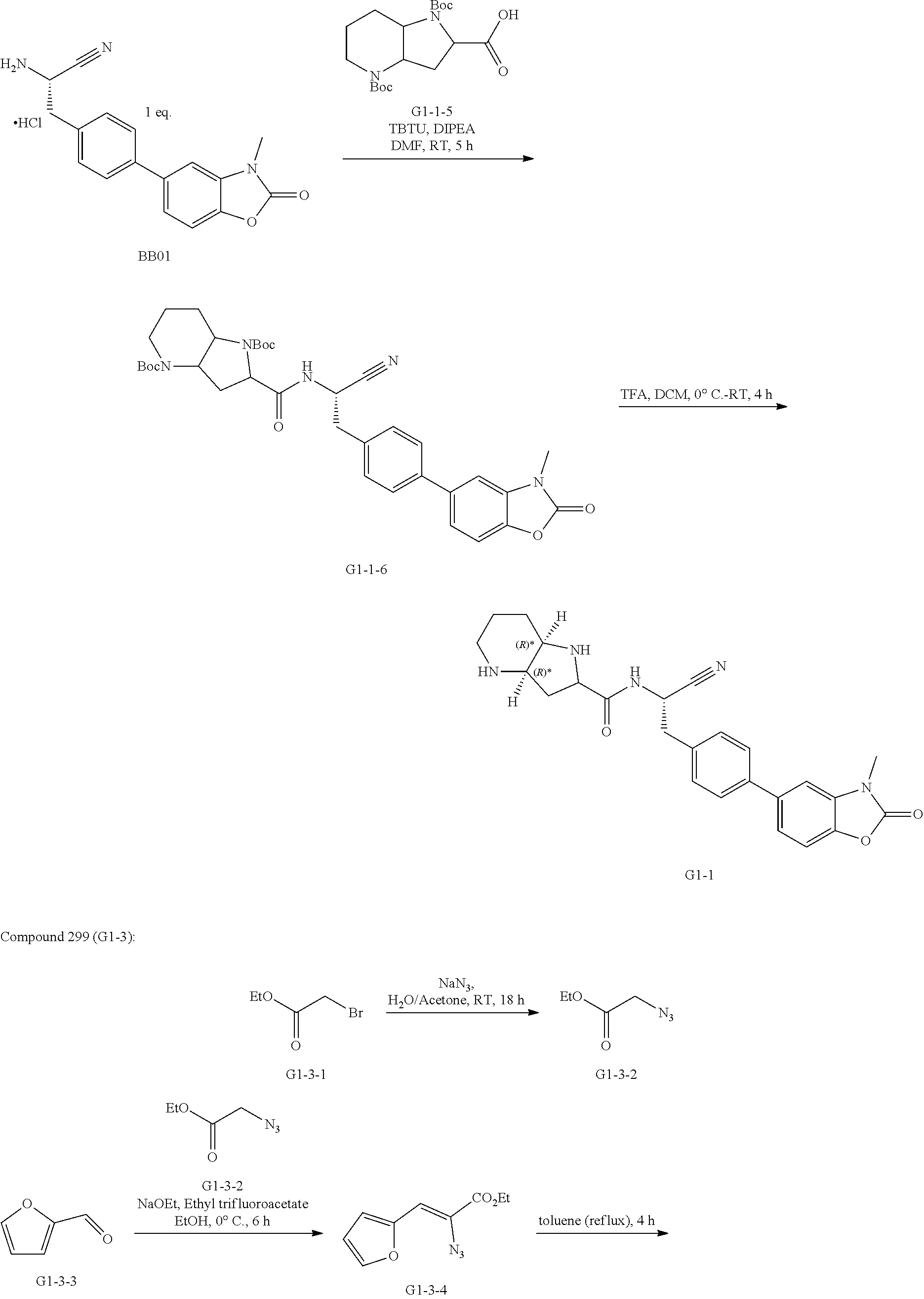

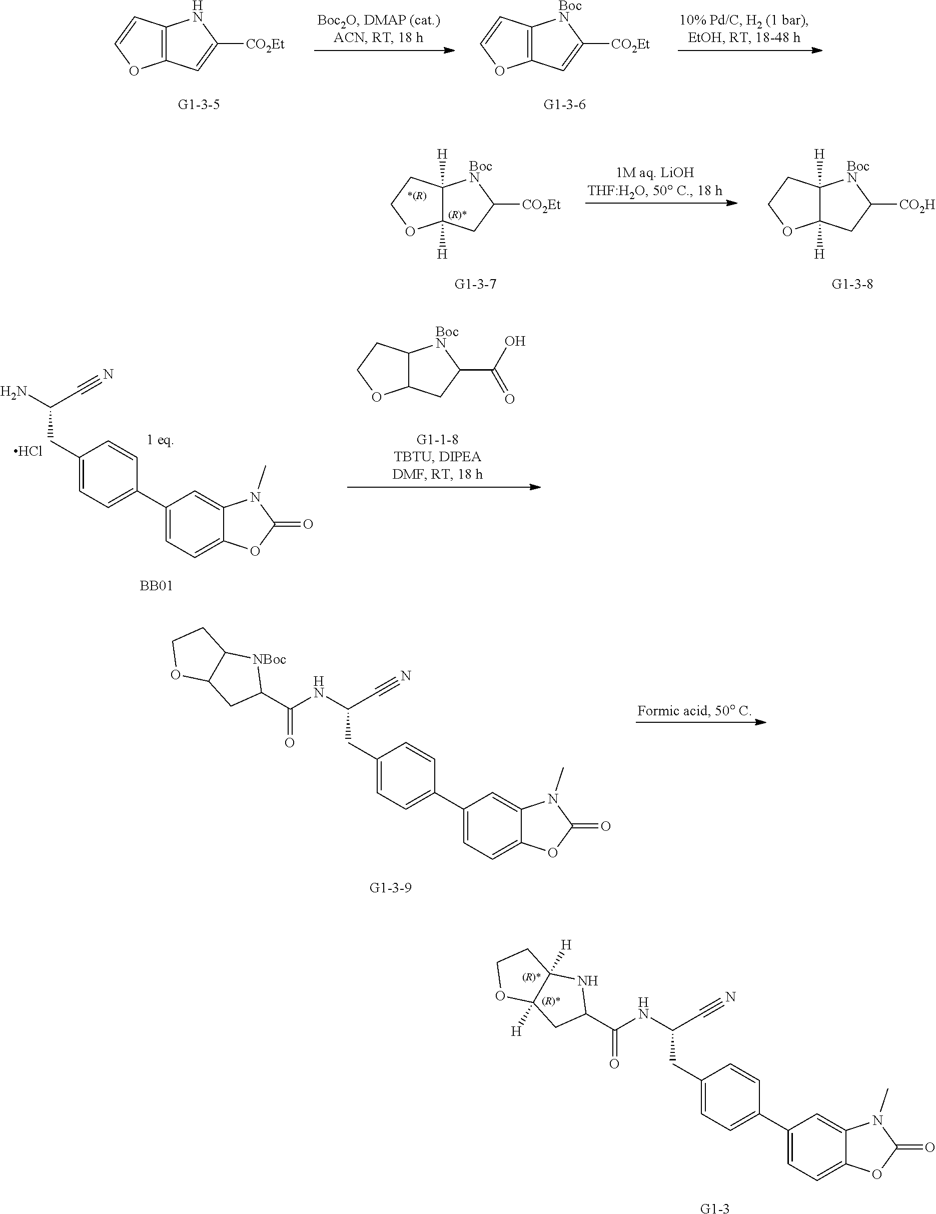

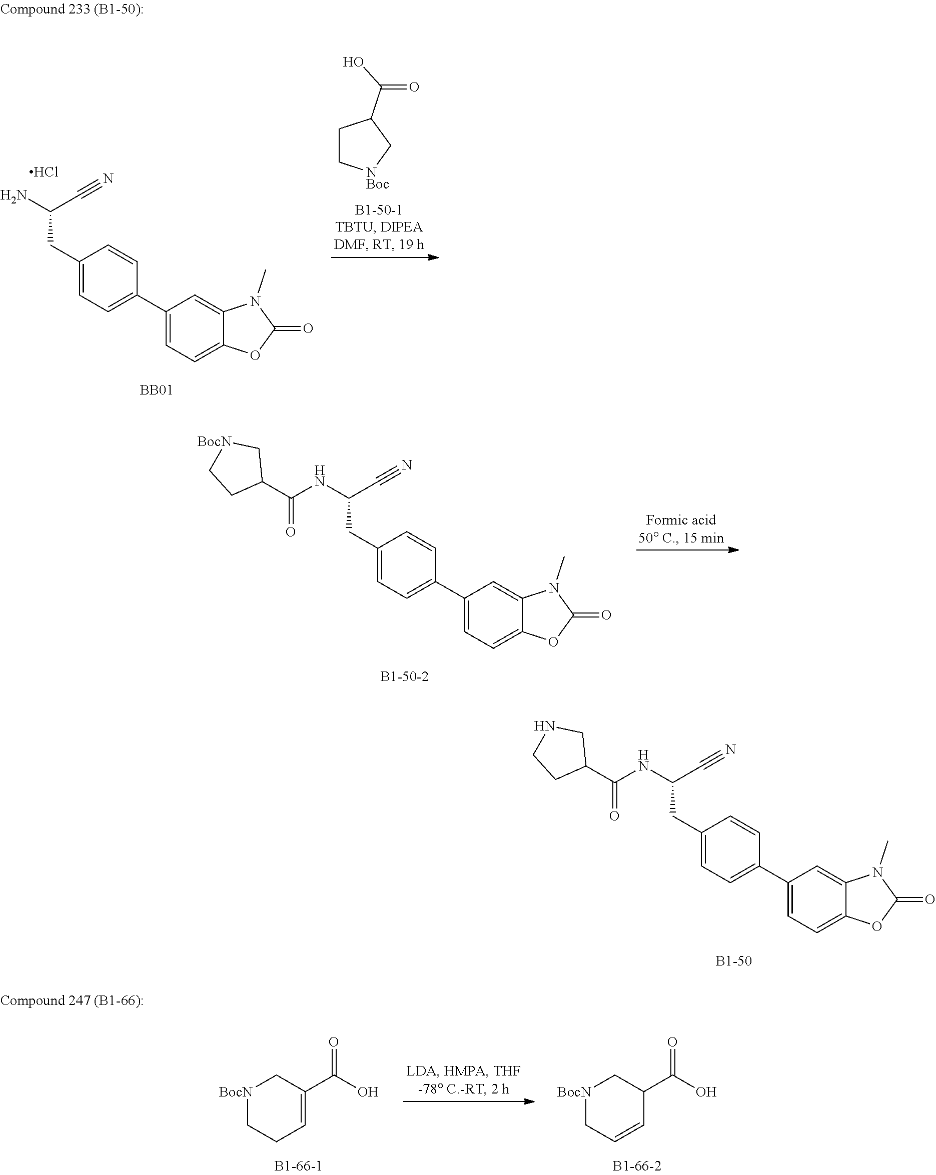

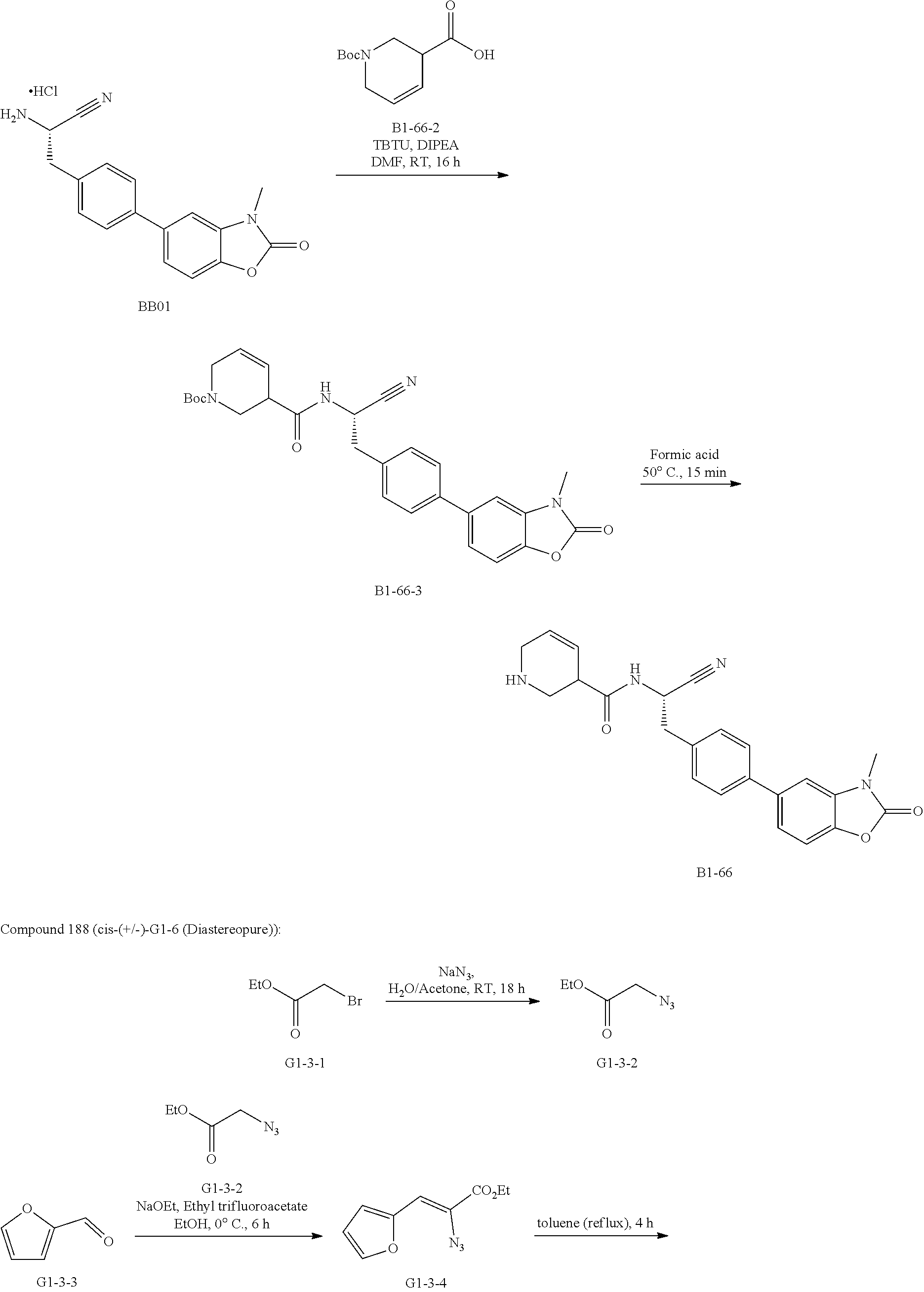

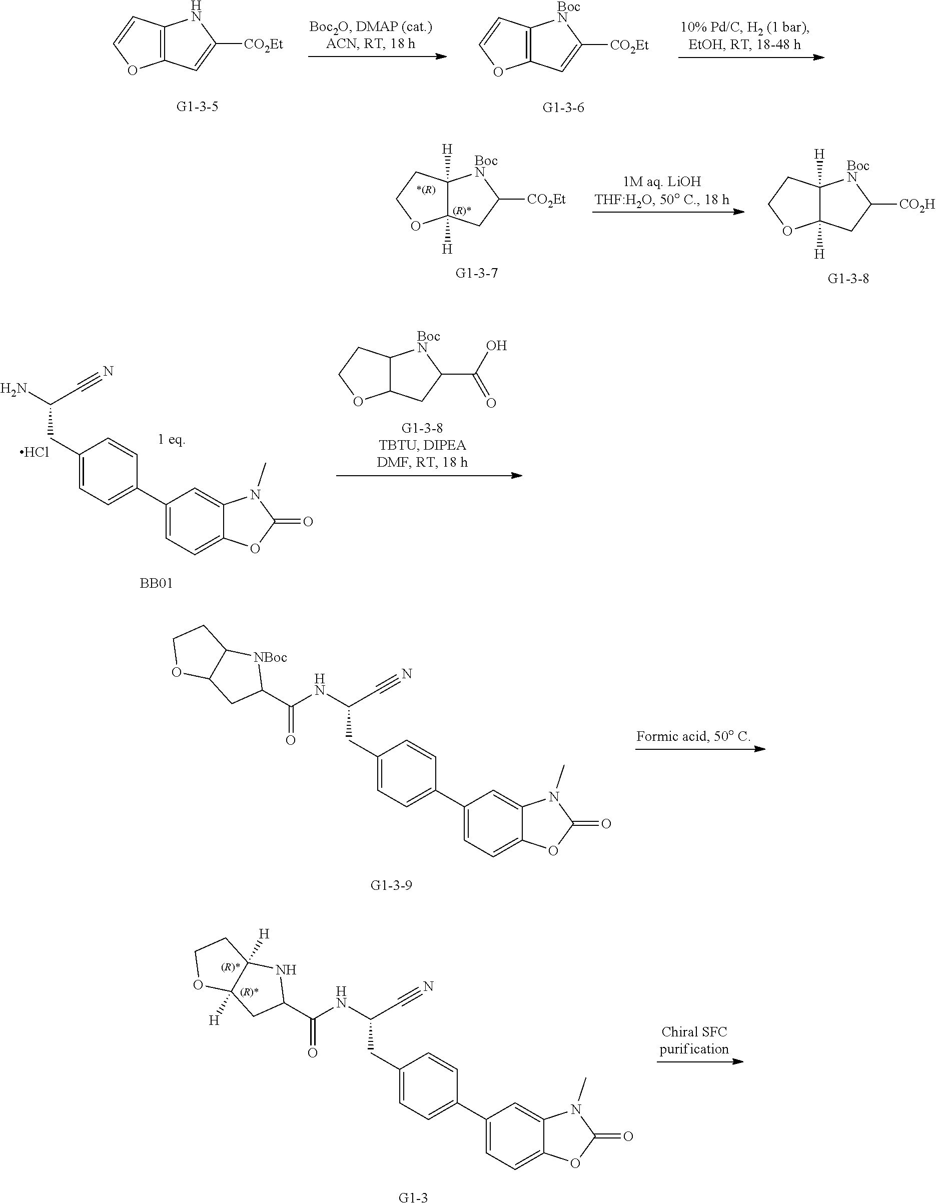

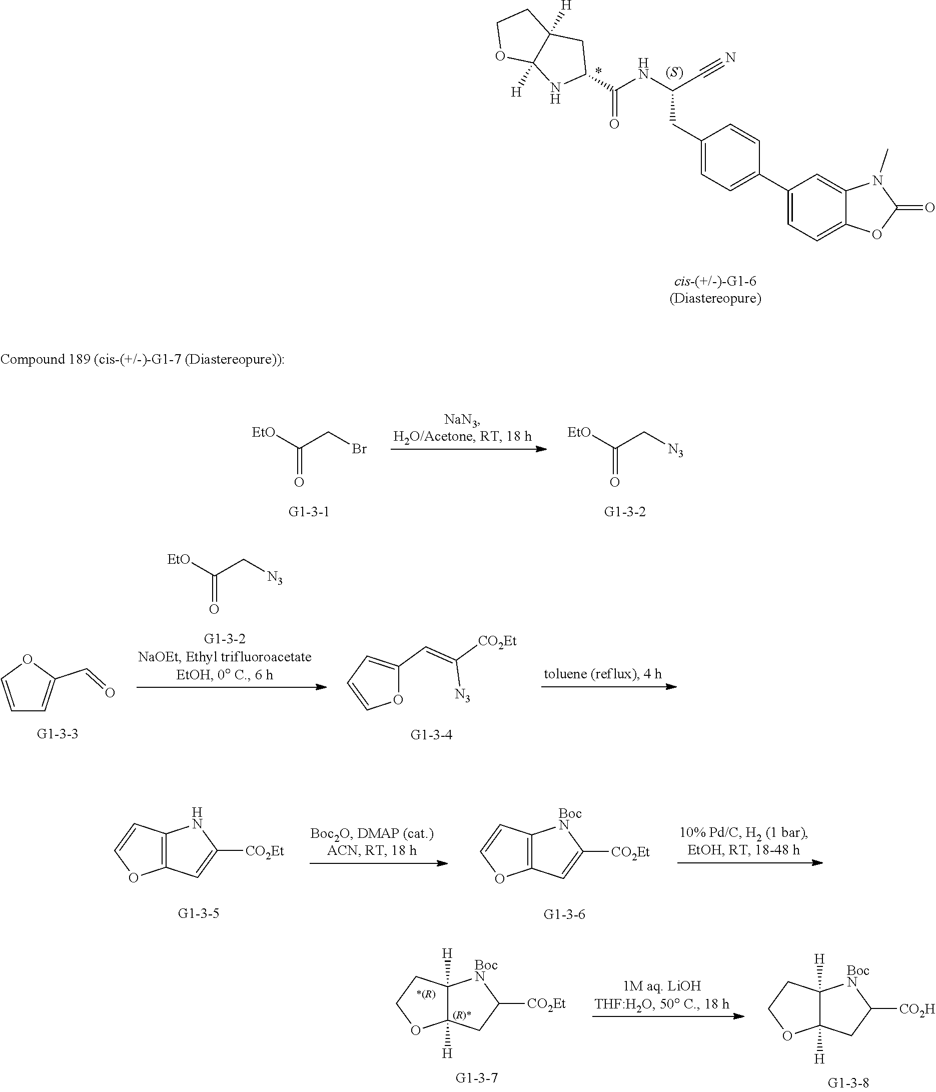

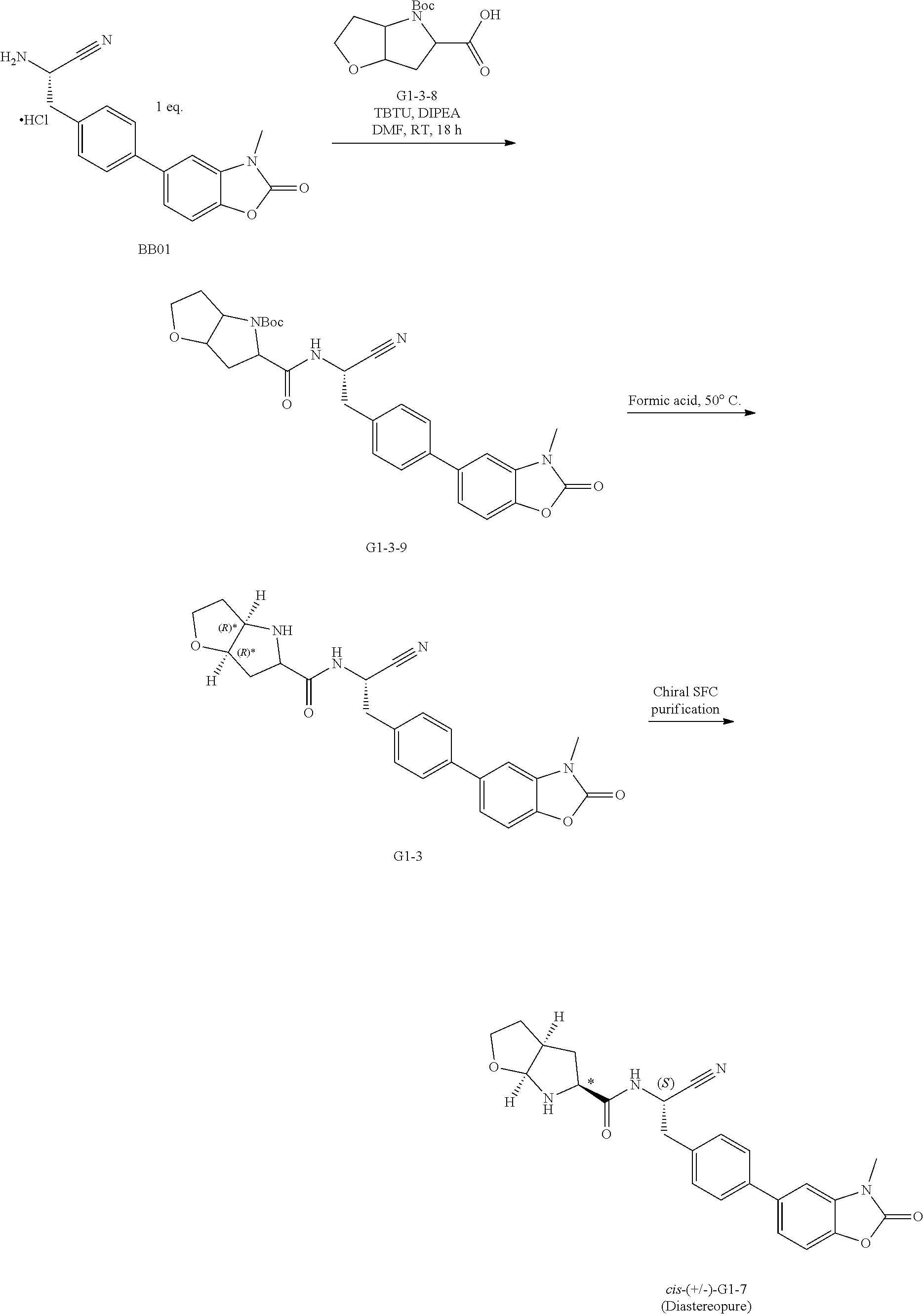

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A compound of Formula (I): 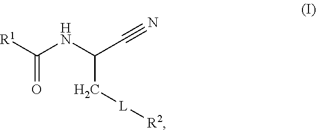
or a pharmaceutically acceptable salt or deuterated form thereof, wherein,
R¹ is 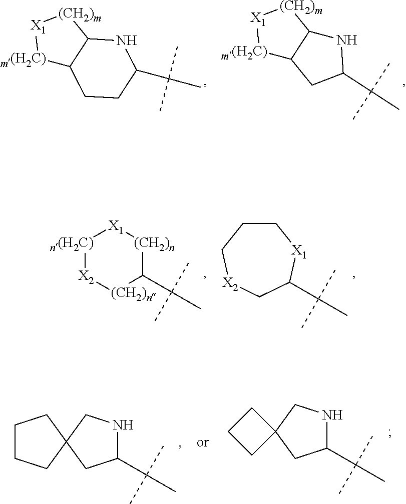
R² is 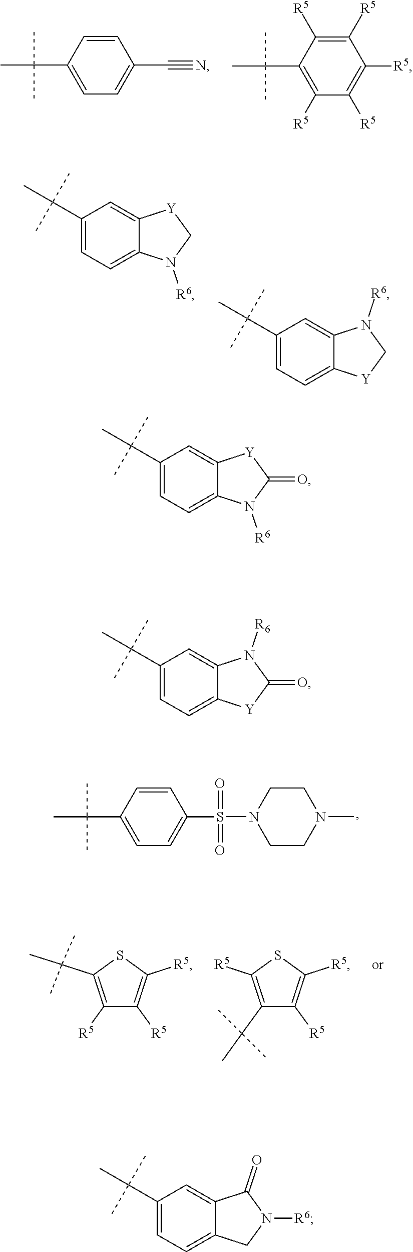
X₁, X₂ and X₃ are independently O, S, NR³ or CR³R⁴;
each R³, R⁴ and R⁵ is independently H, F, Cl, Br, I or C₁-C₆ alkyl;
each R⁶ is independently H or C₁-C₆ alkyl;
each Y is independently O, S, CHR⁶ or NR⁶;
m and m′ are each independently an integer from 0-3, and the total sum of m and m′ is ≤3;
each n, n′ and n″ is independently an integer from 0-3, and the total sum of n, n′ and n″ is ≤4; and
L is 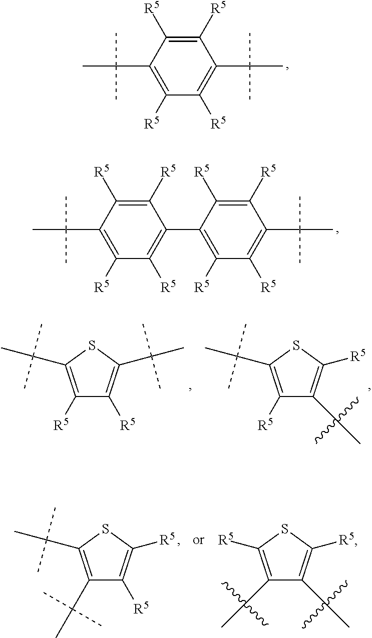
provided that,
(i) when R¹ is 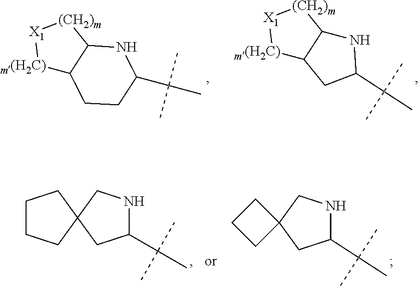
and X₁ is —CH₂—, then L and R² taken together, is not 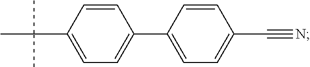
(ii) when R¹ is 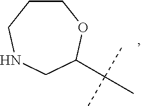
then L and R² taken together, is not 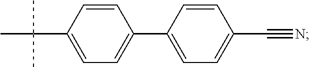
(iii) when R¹ is 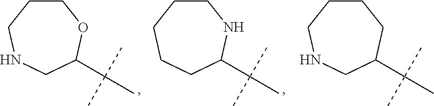
and L is 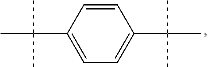
then R² is not 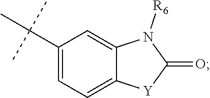
(iv) when R¹ is 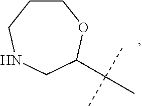
and L is 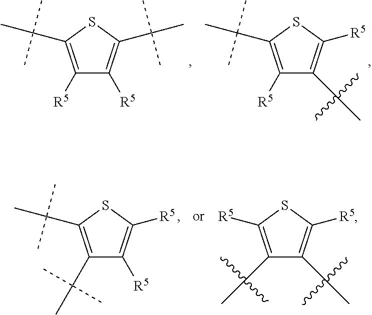
then R² is not 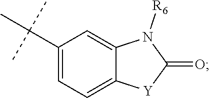
(v) when R¹ is 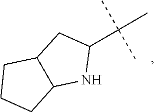
and L is 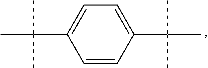
then R² is not 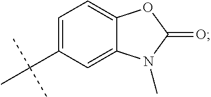
(vi) when R¹ is 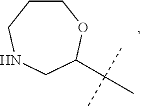
and L is 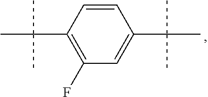
then R² is not 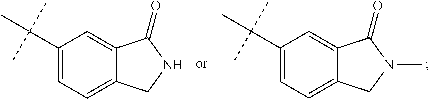
and
(vii) when R¹ is 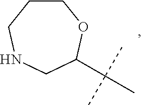
and L is 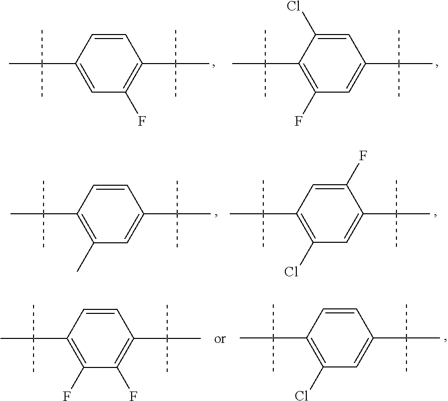
then R² is not

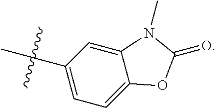 [SMILES: *c1ccc2oc(=O)n(C)c2c1]

2. The compound of claim 1, or a pharmaceutically acceptable salt thereof or deuterated form, wherein,
R¹ is

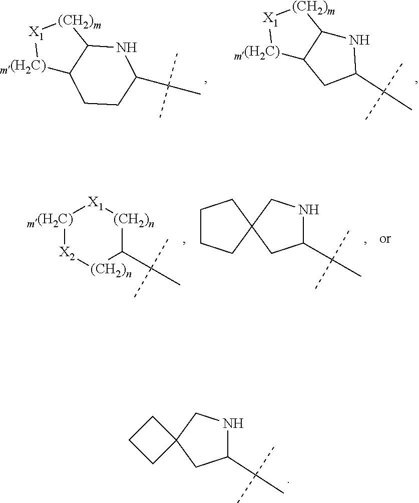

3.-16. (canceled)

17. The compound of claim 1, or a pharmaceutically acceptable salt or deuterated form thereof, wherein X₁ is O or NH.

18.-22. (canceled)

23. The compound of claim 1, or a pharmaceutically acceptable salt or deuterated form thereof, wherein the sum of n, n′ and n″ is 1, 2 or 3.

24.-44. (canceled)

45. The compound of claim 1, or a pharmaceutically acceptable salt or deuterated form thereof, wherein R² is

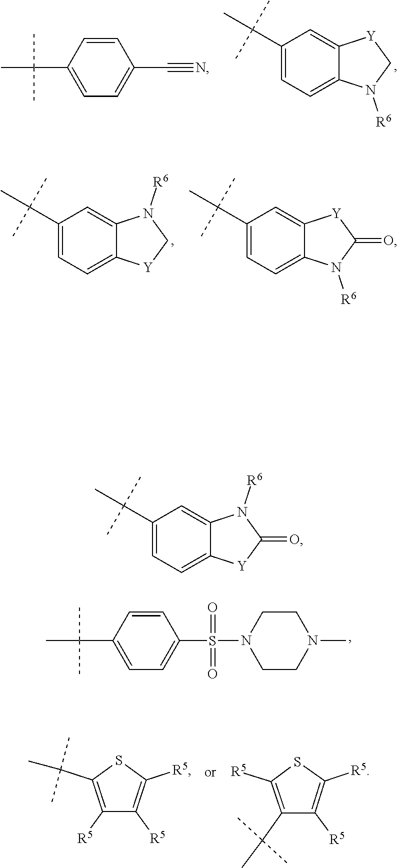

46. The compound of claim 1, or a pharmaceutically acceptable salt or deuterated form thereof, wherein R² is

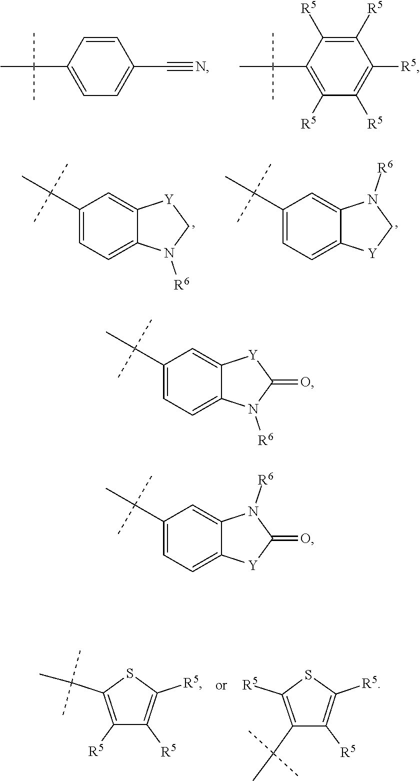

47. (canceled)

48. The compound of claim 1, or a pharmaceutically acceptable salt or deuterated form thereof, wherein R² is 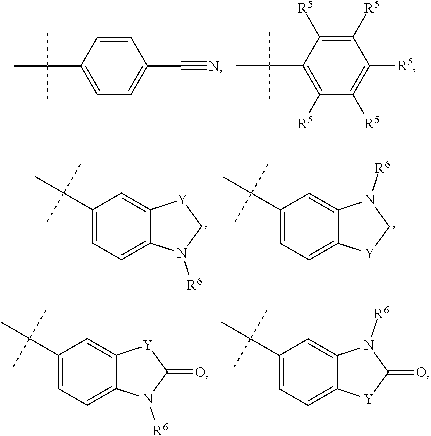
wherein Y is O, CHR⁶ or NR⁶.

49.-51. (canceled)

52. The compound of claim 1, or a pharmaceutically acceptable salt or deuterated form thereof, wherein R² is

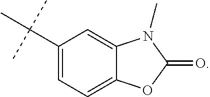

53.-69. (canceled)

70. A compound of Formula (II), or a pharmaceutically acceptable salt or deuterated form thereof: 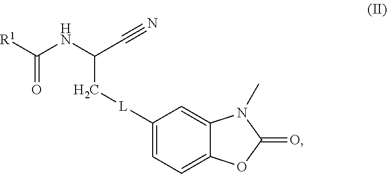
wherein,
R¹ is 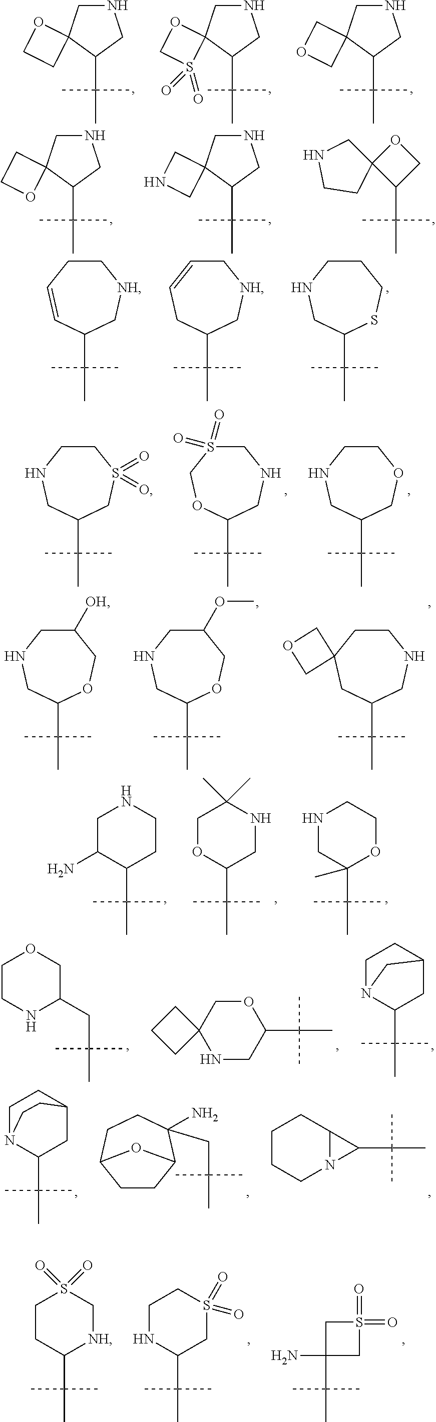 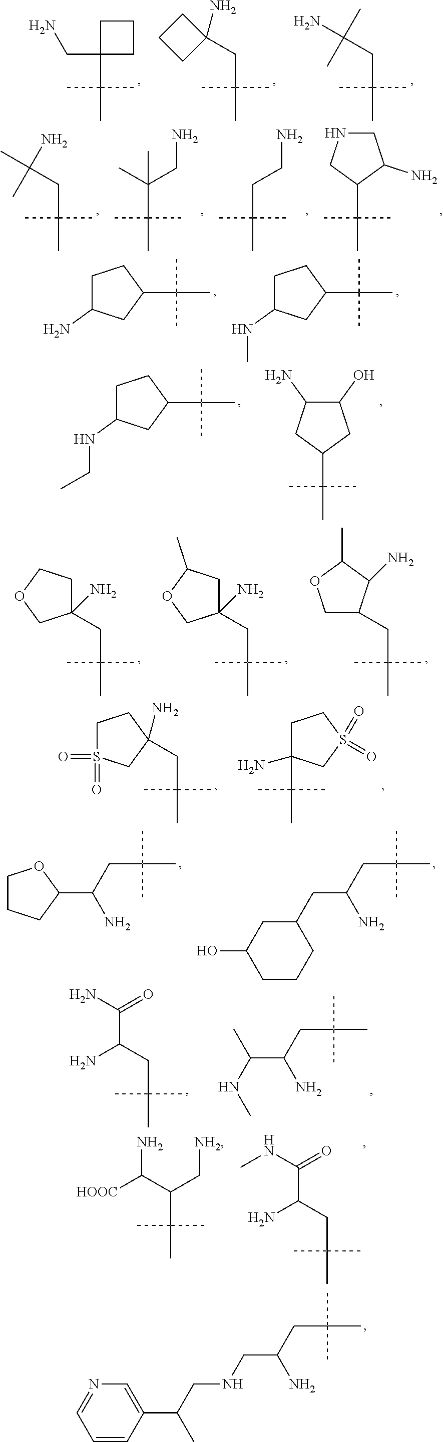 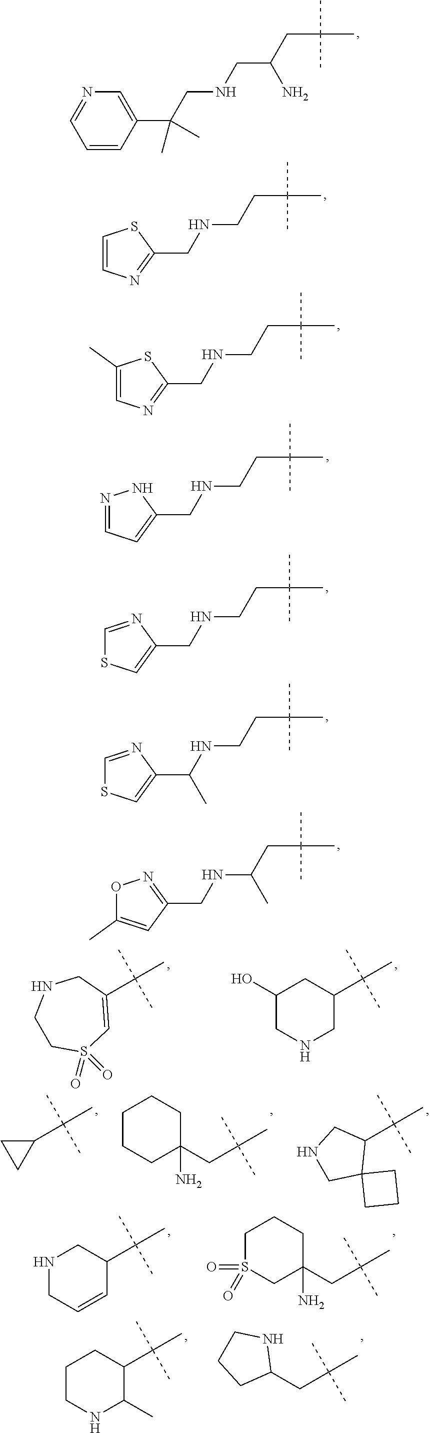 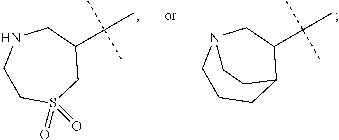
each R² is independently H, F, Cl, Br, I or C₁-C₆ alkyl; and
L is 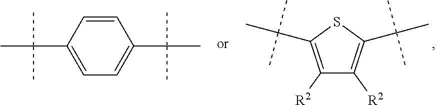
wherein the compound is not

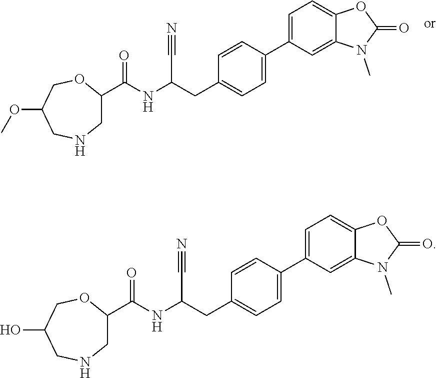 [SMILES: COC1CNCC(C(=O)NC(C#N)Cc2ccc(-c3ccc4oc(=O)n(C)c4c3)cc2)OC1 ; Cn1c(=O)oc2ccc(-c3ccc(CC(C#N)NC(=O)C4CNCC(O)CO4)cc3)cc21]

71.-75. (canceled)

76. The compound of claim 70, or a pharmaceutically acceptable salt or deuterated form thereof, wherein R¹ is

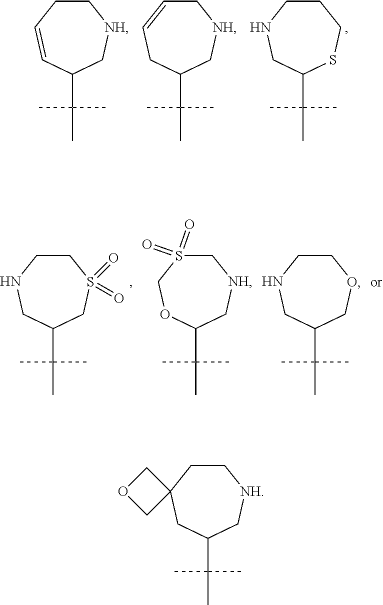

77. The compound of claim 70, or a pharmaceutically acceptable salt or deuterated form thereof, wherein R¹ is

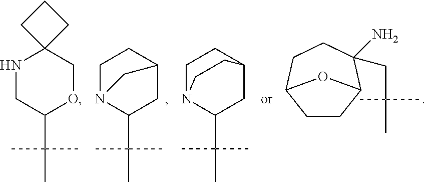

78. The compound of claim 70, or a pharmaceutically acceptable salt or deuterated form thereof, wherein R¹ is

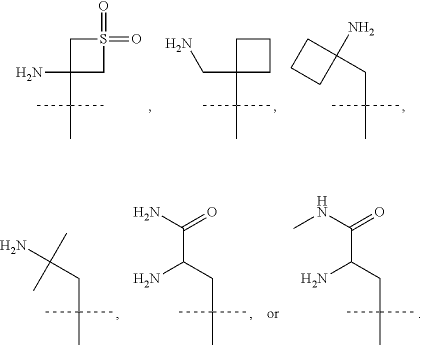

79.-148. (canceled)

149. The compound of claim 70, or a pharmaceutically accept salt or deuterated form thereof, wherein the compound is:

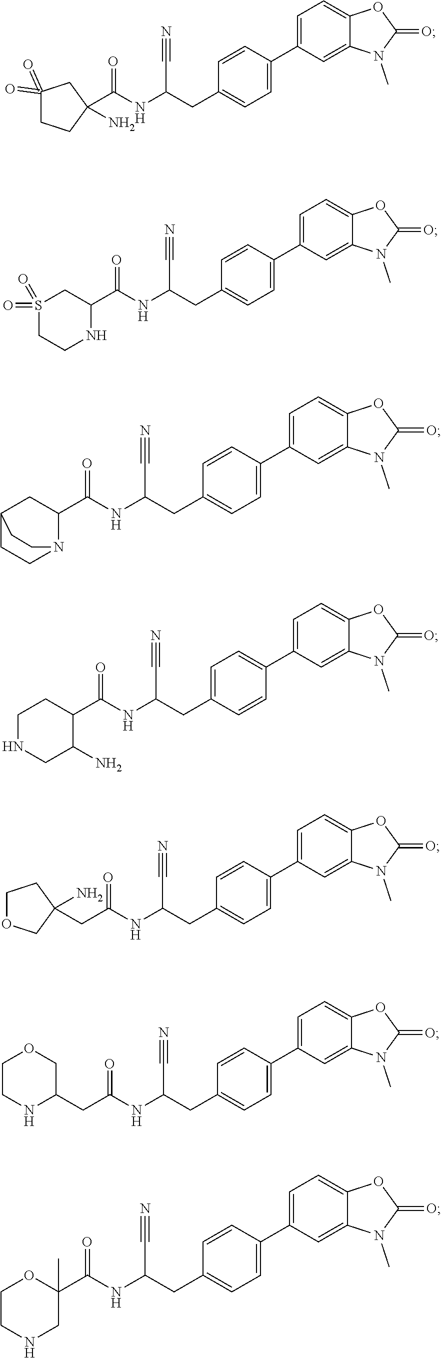

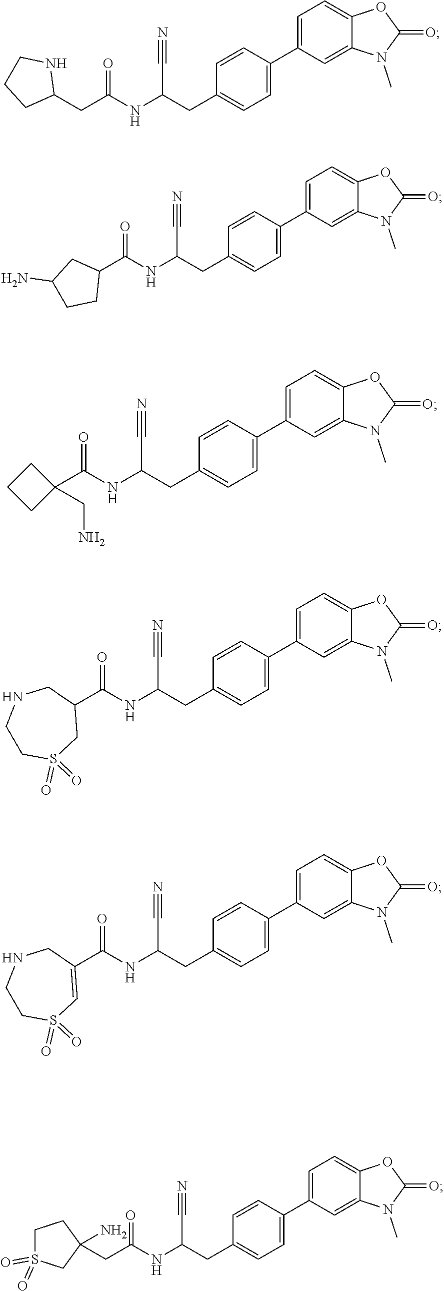 [SMILES: Cn1c(=O)oc2ccc(-c3ccc(CC(C#N)NC(=O)CC4CCCN4)cc3)cc21 ; Cn1c(=O)oc2ccc(-c3ccc(CC(C#N)NC(=O)C4CCC(N)C4)cc3)cc21 ; Cn1c(=O)oc2ccc(-c3ccc(CC(C#N)NC(=O)C4(CN)CCC4)cc3)cc21 ; Cn1c(=O)oc2ccc(-c3ccc(CC(C#N)NC(=O)C4CNCCS(=O)(=O)C4)cc3)cc21 ; Cn1c(=O)oc2ccc(-c3ccc(CC(C#N)NC(=O)C4=CS(=O)(=O)CCNC4)cc3)cc21 ; Cn1c(=O)oc2ccc(-c3ccc(CC(C#N)NC(=O)CC4(N)CCS(=O)(=O)C4)cc3)cc21]

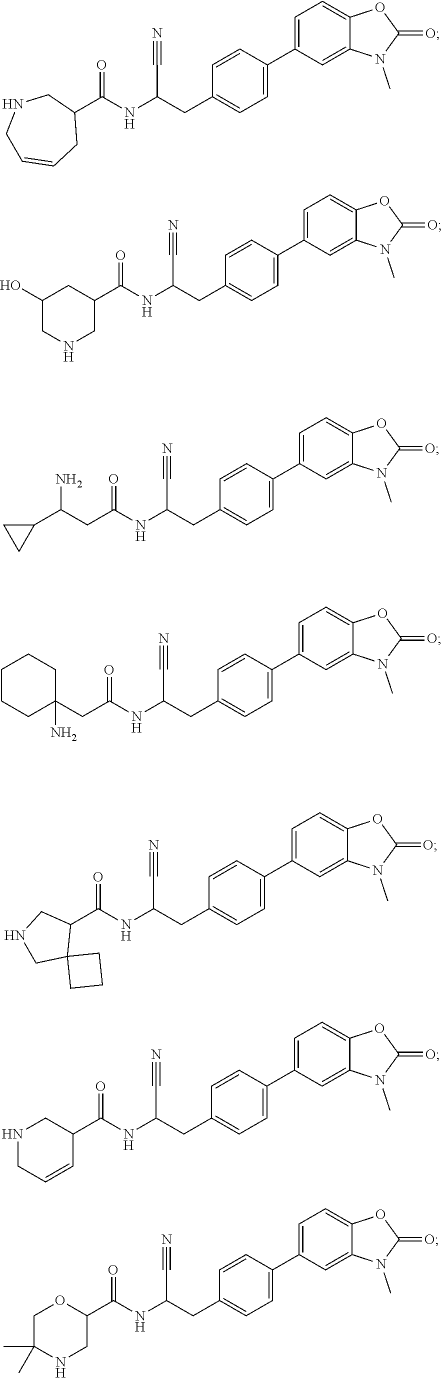

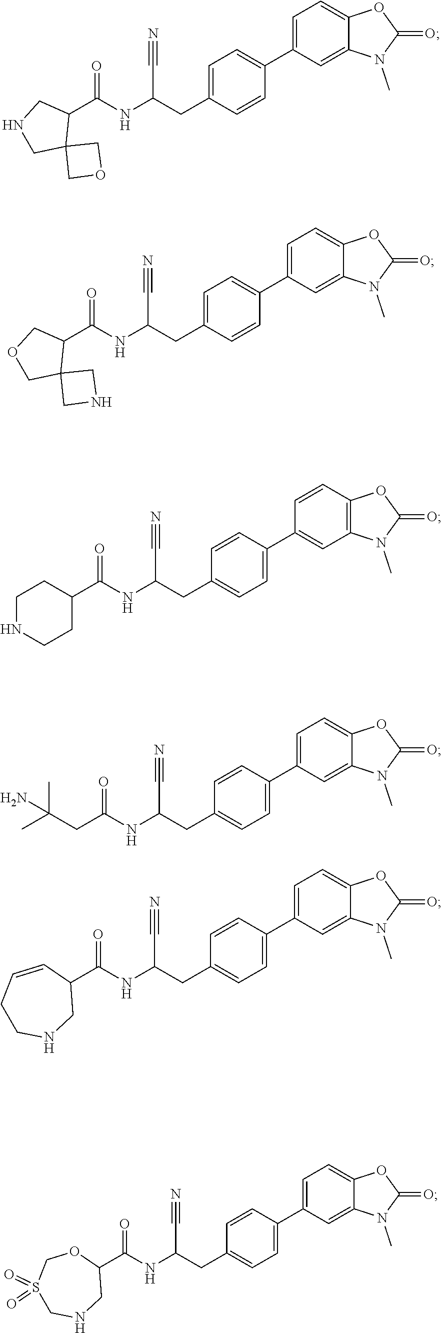 [SMILES: Cn1c(=O)oc2ccc(-c3ccc(CC(C#N)NC(=O)C4CNCC45COC5)cc3)cc21 ; Cn1c(=O)oc2ccc(-c3ccc(CC(C#N)NC(=O)C4COCC45CNC5)cc3)cc21 ; Cn1c(=O)oc2ccc(-c3ccc(CC(C#N)NC(=O)C4CCNCC4)cc3)cc21 ; Cn1c(=O)oc2ccc(-c3ccc(CC(C#N)NC(=O)CC(C)(C)N)cc3)cc21 ; Cn1c(=O)oc2ccc(-c3ccc(CC(C#N)NC(=O)C4C=CCCNC4)cc3)cc21 ; Cn1c(=O)oc2ccc(-c3ccc(CC(C#N)NC(=O)C4CNCS(=O)(=O)CO4)cc3)cc21]

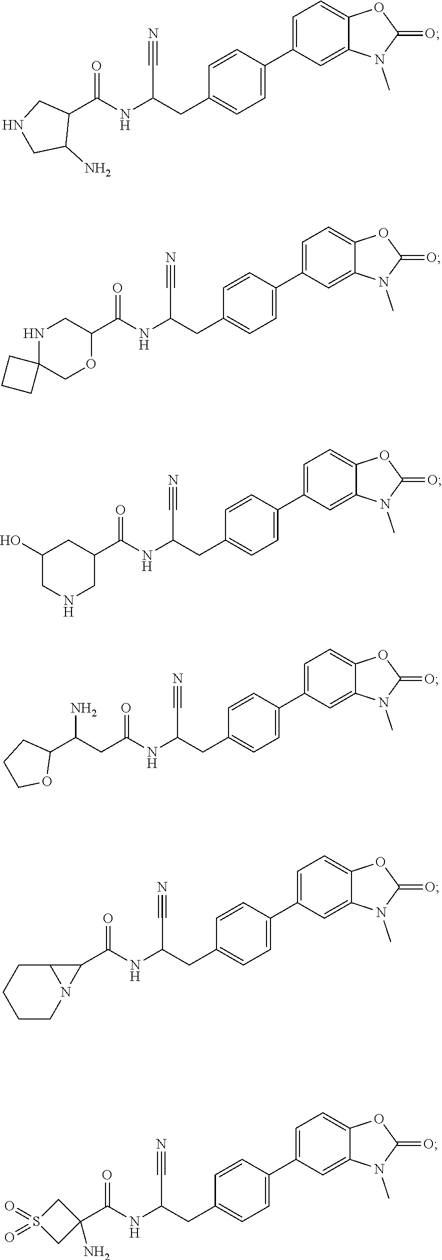 [SMILES: Cn1c(=O)oc2ccc(-c3ccc(CC(C#N)NC(=O)C4CNCC4N)cc3)cc21 ; Cn1c(=O)oc2ccc(-c3ccc(CC(C#N)NC(=O)C4CNC5(CCC5)CO4)cc3)cc21 ; Cn1c(=O)oc2ccc(-c3ccc(CC(C#N)NC(=O)C4CNCC(O)C4)cc3)cc21 ; Cn1c(=O)oc2ccc(-c3ccc(CC(C#N)NC(=O)CC(N)C4CCCO4)cc3)cc21 ; Cn1c(=O)oc2ccc(-c3ccc(CC(C#N)NC(=O)C4C5CCCCN54)cc3)cc21 ; Cn1c(=O)oc2ccc(-c3ccc(CC(C#N)NC(=O)C4(N)CS(=O)(=O)C4)cc3)cc21]

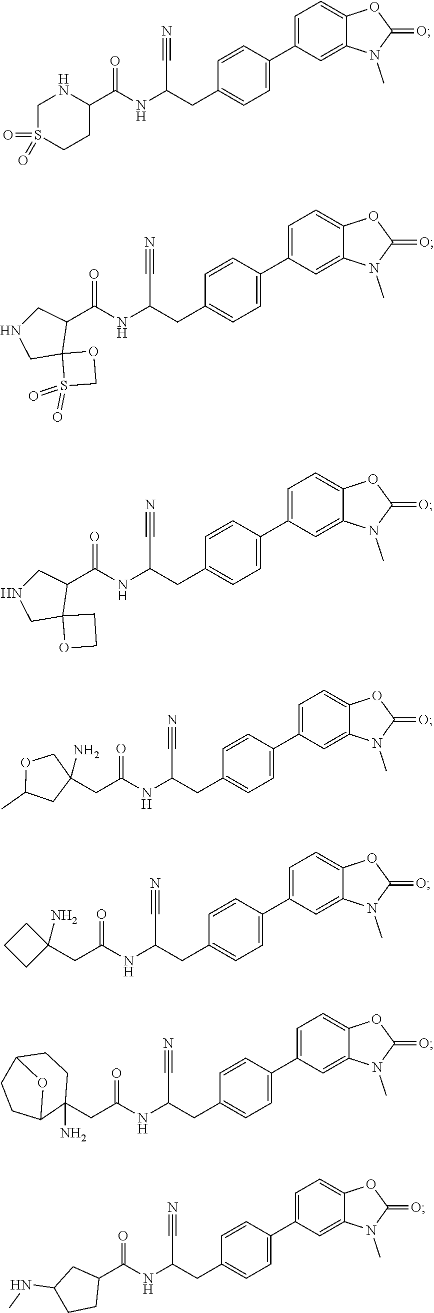

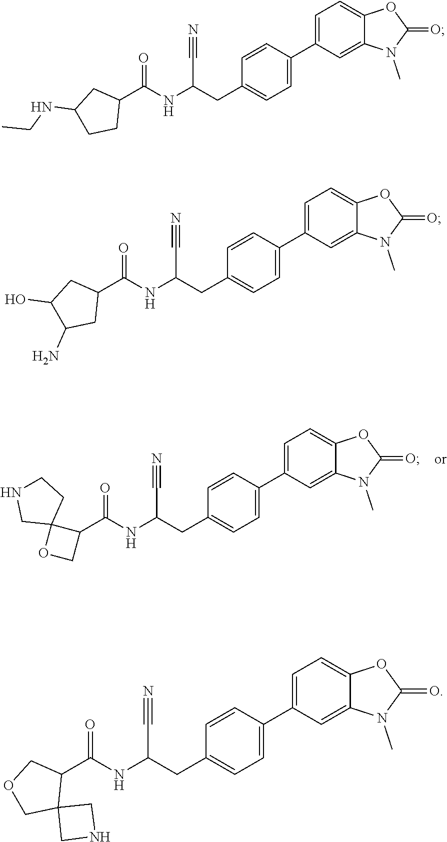 [SMILES: CCNC1CCC(C(=O)NC(C#N)Cc2ccc(-c3ccc4oc(=O)n(C)c4c3)cc2)C1 ; Cn1c(=O)oc2ccc(-c3ccc(CC(C#N)NC(=O)C4CC(N)C(O)C4)cc3)cc21 ; Cn1c(=O)oc2ccc(-c3ccc(CC(C#N)NC(=O)C4COC45CCNC5)cc3)cc21 ; Cn1c(=O)oc2ccc(-c3ccc(CC(C#N)NC(=O)C4COCC45CNC5)cc3)cc21]

150.-171. (canceled)

172. A compound of Formula (I-I): 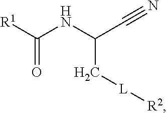
or a pharmaceutically acceptable salt or deuterated form thereof, wherein,
R¹ is 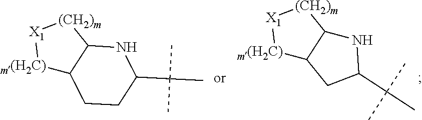
R² is 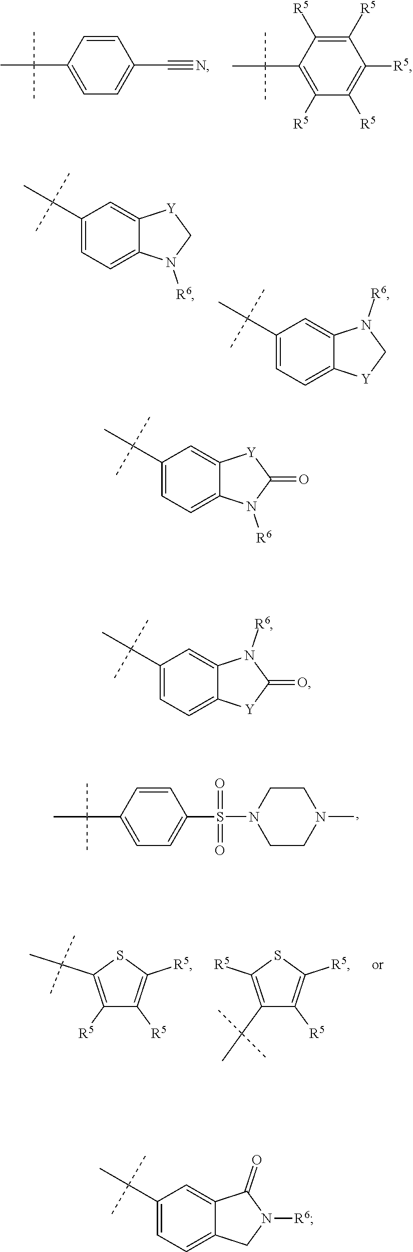
X₁, X₂ and X₃ are independently O, S, NR³ or CR³R⁴;
each R³, R⁴ and R⁵ is independently H, F, Cl, Br, I or C₁-C₆ alkyl;
each R⁶ is independently H or C₁-C₆ alkyl;
each Y is independently O, S, CHR⁶ or NR⁶;
m and m′ are each independently an integer from 0-3, and the total sum of m and m′ is ≤3; and
L is 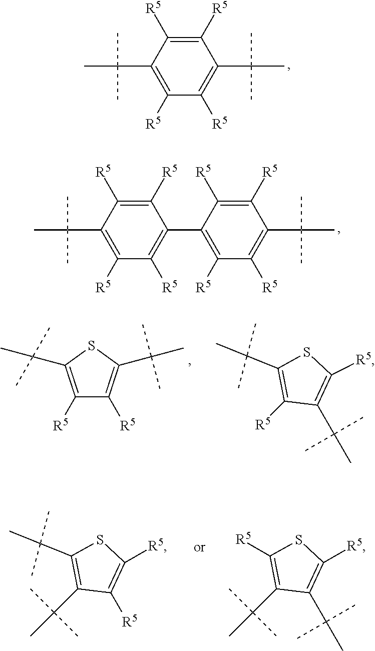
provided that, the compound is not:

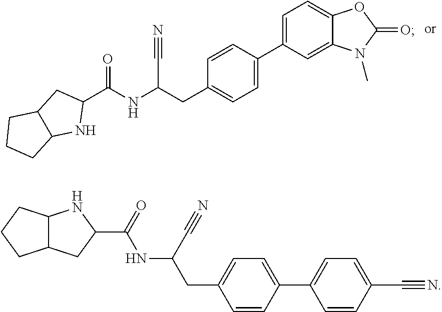 [SMILES: Cn1c(=O)oc2ccc(-c3ccc(CC(C#N)NC(=O)C4CC5CCCC5N4)cc3)cc21 ; "or" ; N#Cc1ccc(-c2ccc(CC(C#N)NC(=O)C3CC4CCCC4N3)cc2)cc1]

173. The compound of claim 172, or a pharmaceutically acceptable salt or deuterated form thereof, having the following chemical structure:

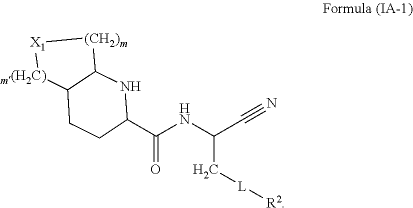

174. The compound of claim 172, or a pharmaceutically acceptable salt or deuterated form thereof, having the following chemical structure:

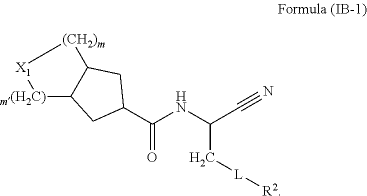

175. The compound of claim 172, or a pharmaceutically acceptable salt or deuterated form thereof, having the following chemical structure: 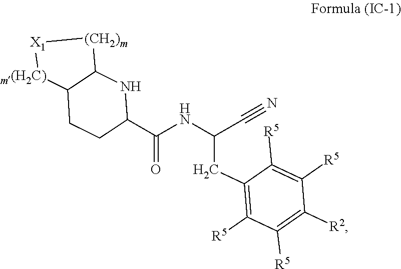
wherein at least one R₅ is F.

176. The compound of claim 172, or a pharmaceutically acceptable salt or deuterated form thereof, having the following chemical structure: 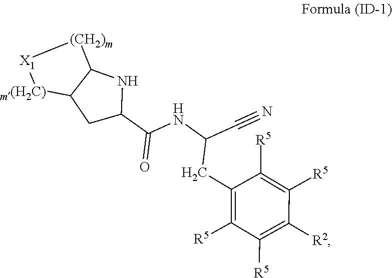
wherein at least one R₅ is F.

177. The compound of claim 172, or a pharmaceutically acceptable salt or deuterated form thereof, wherein,
R¹ is

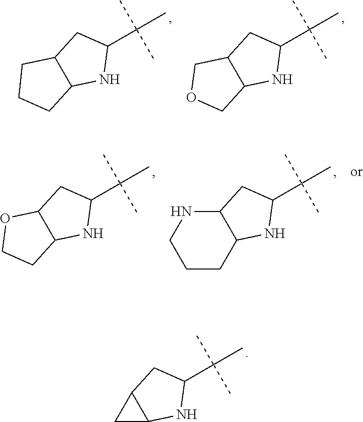

178-222. (canceled)

223. The compound of claim 1, or a pharmaceutically acceptable salt or deuterated form thereof selected from one of the compounds of Table 3.

224.-328. (canceled)

329. A method for treating an obstructive disease of the airway in a patient in need thereof, comprising, administering to the patient for an administration period, a composition comprising an effective amount of a compound of claim 1.

330. A method for treating an obstructive disease of the airway in a patient in need thereof, comprising, administering to the patient for an administration period, a composition comprising an effective amount of a compound of claim 70.

331. A method for treating an obstructive disease of the airway in a patient in need thereof, comprising, administering to the patient for an administration period, a composition comprising an effective amount of a compound of claim 172.
